(19) United States
(12) Patent Application Publication
(10) Pub. No.: US 2009/0311702 A1
(43) Pub. Date: Dec. 17, 2009

(54) TESTS TO PREDICT RESPONSIVENESS OF CANCER PATIENTS TO CHEMOTHERAPY TREATMENT OPTIONS

(76) Inventors: STEVE SHAK, REDWOOD CITY, CA (US); JOFFRE B. BAKER, MONTARA, CA (US); CARL YOSHIZAWA, REDWOOD CITY, CA (US); JOSEPH SPARANO, PLEASANTVILLE, NY (US); ROBERT GRAY, BOSTON, MA (US)

Correspondence Address: Genomic Health, Inc.;c/o Kathleen Determann, 301 Penobscot Road, Redwood City, CA, 94063, US

(21) Appl. No.: 12/464,797

(22) Filed: May 12, 2009

Related U.S. Application Data
(60) Provisional application No. 61/052,573, filed on May 12, 2008.
(60) Provisional application No. 61/057,182, filed on May 29, 2008.

(51) Int. Cl. C12Q 1/68; C12Q 1/02; G01N 1/30

(52) U.S. Cl. 435/6; 435/29; 435/40.52

(57) ABSTRACT

The present disclosure provides methods and compositions to facilitate prediction of the likelihood of responsiveness of cancer patients to treatment including a taxane and/or a cyclophosphamide.

### CROSS-REFERENCE TO RELATED APPLICATIONS

This application claims priority benefit of U.S. Provisional Application Ser. Nos. 61/052,573, filed May 12, 2008, and 61/057,182 filed May 29, 2008, the entire disclosures of which are incorporated herein by reference in their entirety.

### TECHNICAL FIELD

The present invention provides genes and gene sets, the expression levels of which are useful for predicting response of cancer patients to chemotherapy. The invention further concerns tests using such molecular markers, arrays and kits for use in such methods, and reports comprising the results and/or conclusions of such tests.

### INTRODUCTION

For many patients with cancer, treatment may include surgical resection of the tumor, hormonal therapy, and chemotherapy. A range of chemotherapy choices are available. Ideally, the choice for an individual patient takes into account both the risk of cancer recurrence and the likelihood that the patient will respond to the chemotherapy chosen.

One critical issue in treatment of breast cancer is the identification of which patients are likely to respond to a standard chemotherapy (e.g. an anthracycline and a cyclophosphamide) and which patients are less likely to respond to standard chemotherapy and should therefore be considered for more aggressive chemotherapy (e.g., a chemotherapy regimen that includes a taxane). Currently, no satisfactory tests are available for identifying patients more likely to respond to standard chemotherapy as opposed to treatment with a taxane-containing treatment regimen.

### SUMMARY

The present disclosure provides methods and compositions to facilitate prediction of the likelihood of responsiveness of cancer patients to treatment including a taxane and/or a cyclophosphamide.

The present disclosure provides methods of predicting whether a hormone receptor (HR) positive cancer patient will exhibit a beneficial response to chemotherapy, where the method involves measuring an expression level of a gene, or its expression product, in a tumor sample obtained from the patient, wherein the gene is selected from the group consisting of ABCC1, ABCC5, ABCD1, ACTB, ACTR2, AKT1, AKT2, APC, APOC1, APOE, APRT, BAK1, BAX, BBC3, BCL2L11, BCL2L13, BID, BUB1, BUB3, CAPZA1, CCT3, CD14, CDC25B, CDCA8, CHEK2, CHFR, CSNK1D, CST7, CXCR4, DDR1, DICER1, DUSP1, ECGF1, EIF4E2, ERBB4, ESR1, FAS, GADD45B, GATA3, GCLC, GDF15, GNS, HDAC6, HSPA1A, HSPA1B, HSPA9B, IL7, ILK, LAPTM4B, LILRB1, LIMK2, MAD2L1BP, MAP2K3, MAPK3, MAPRE1, MCL1, MRE11A, NEK2, NFKB1, NME6, NTSR2, PLAU, PLD3, PPP2CA, PRDX1, PRKCH, RAD1, RASSF1, RCC1, REG1A, RELA, RHOA, RHOB, RPN2, RXRA, SHC1, SIRT1, SLC1A3, SLC35B1, SRC, STK10, STMN1, TBCC, TBCD, TNFRSF10A, TOP3B, TSPAN4, TUBA3, TUBA6, TUBB, TUBB2C, UFM1, VEGF, VEGFB, VHL, ZW10, and ZWILCH; using the expression level to determine a likelihood of a beneficial response to a treatment including a taxane, wherein expression of DDR1, EIF4E2, TBCC, STK10, ZW10, BBC3, BAX, BAK1, TSPAN4, SLC1A3, SHC1, CHFR, RHOB, TUBA6, BCL2L13, MAPRE1, GADD45B, HSPA1B, FAS, TUBB, HSPA1A, MCL1, CCT3, VEGF, TUBB2C, AKT1, MAD2L1BP, RPN2, RHOA, MAP2K3, BID, APOE, ESR1, ILK, NTSR2, TOP3B, PLD3, DICER1, VHL, GCLC, RAD1, GATA3, CXCR4, NME6, UFM1, BUB3, CD14, MRE11A, CST7, APOC1, GNS, ABCC5, AKT2, APRT, PLAU, RCC1, CAPZA1, RELA, NFKB1, RASSF1, BCL2L11, CSNK1D, SRC, LIMK2, SIRT1, RXRA, ABCD1, MAPK3, DUSP1, ABCC1, PRKCH, PRDX1, TUBA3, VEGFB, LILRB1, LAPTM4B, HSPA9B, ECGF1, GDF15, ACTR2, IL7, HDAC6, CHEK2, REG1A, APC, SLC35B1, ACTB, PPP2CA, TNFRSF10A, TBCD, ERBB4, CDC25B, or STMN1 is positively correlated with increased likelihood of a beneficial response to a treatment including a taxane, and wherein expression of CDCA8, ZWILCH, NEK2, or BUB1 is negatively correlated with an increased likelihood of a beneficial response to a treatment including a taxane; and generating a report including information based on the likelihood of a beneficial response to chemotherapy including a taxane.

The methods can further involves using a gene expression level to determine a likelihood of a beneficial response to a treatment including a cyclophosphamide, wherein expression of ZW10, BAX, GADD45B, FAS, ESR1, NME6, MRE11A, AKT2, RELA, RASSF1, PRKCH, VEGFB, LILRB1, ACTR2, REG1A, or PPP2CA is positively correlated with increased likelihood of a beneficial response to a treatment including a cyclophosphamide, and wherein expression of DDR1, EIF4E2, TBCC, STK10, BBC3, BAK1, TSPAN4, SHC1, CHFR, RHOB, TUBA6, BCL2L13, MAPRE1, HSPA1, TUBB, HSPA1A, MCL1, CCT3, VEGF, TUBB2C, AKT1, MAD2L1BP, RPN2, RHOA, MAP2K3, BID, APOE, ILK, NTSR2, TOP3B, PLD3, DICER1, VHL, GCLC, RAD1, GATA3, CXCR4, UFM1, BUB3, CD14, CST7, APOC1, GNS, ABCC5, APRT, PLAU, RCC1, CAPZA1, NFKB1, BCL2L11, CSNK1D, SRC, LIMK2, SIRT1, RXRA, ABCD1, MAPK3, CDCA8, DUSP1, ABCC1, PRDX1, TUBA3, LAPTM4B, HSPA9B, ECGF1, GDF15, IL7, HDAC6, ZWILCH, CHEK2, APC, SLC35B1, NEK2, ACTB, BUB1, TNFRSF10A, TBCD, ERBB4, CDC25B, or STMN1 is negatively correlated with an increased likelihood of a beneficial response to a treatment including a cyclophosphamide, and wherein the report includes information based on the likelihood of a beneficial response to chemotherapy including a cyclophosphamide.

The chemotherapy can include an anthracycline. The anthracycline can be doxorubicin. Where the chemotherapy is a taxane, the taxane can be docetaxel.

The methods can accomplish measuring of the gene expression level by quantitative PCR. The methods can accomplish measuring of the gene expression level by detection of an intron-based sequence of an RNA transcript of the gene, wherein the expression of which correlates with the expression of a corresponding exon sequence.

The tumor sample can be a formalin-fixed and paraffin-embedded (FPE) or a frozen tumor section.

The methods of the present disclosure includes methods of predicting whether a hormone receptor (HR) positive cancer patient will exhibit a beneficial response to chemotherapy, the methods involve measuring an expression level of a gene, or its expression product, in a tumor sample obtained from the patient, wherein the gene is selected from the group consisting of ABCA9, ABCC1, ABCC10, ABCC3, ABCD1, ACTB, ACTR2, ACTR3, AKT1, AKT2, APC, APEX1, APOC1, APOE, APRT, BAD, BAK1, BAX, BBC3, BCL2, BCL2L1, BCL2L11, BCL2L13, BID, BIRC3, BIRC4, BUB3, CAPZA1, CCT3, CD14, CD247, CD63, CD68, CDC25B, CHEK2, CHFR, CHGA, COL1A1, COL6A3, CRABP1, CSNK1D, CST7, CTSD, CXCR4, CYBA, CYP1B1, DDR1, DIABLO, DICER1, DUSP1, ECGF1, EIF4E2, ELP3, ERBB4, ERCC1, ESR1, FAS, FLAD1, FOS, FOXA1, FUS, FYN, GADD45B, GATA3, GBP1, GBP2, GCLC, GGPS1, GNS, GPX1, HDAC6, HRAS, HSPA1A, HSPA1B, HSPA5, HSPA9B, IGFBP2, IL2RA, IL7, ILK, KDR, KNS2, LAPTM4B, LILRB1, LIMK1, LIMK2, MAD1L1, MAD2L1BP, MAD2L2, MAP2K3, MAP4, MAPK14, MAPK3, MAPRE1, MCL1, MGC52057, MGMT, MMP11, MRE11A, MSH3, NFKB1, NME6, NPC2, NTSR2, PDGFRB, PECAM1, PIK3C2A, PLAU, PLD3, PMS1, PPP2CA, PRDX1, PRKCD, PRKCH, PTEN, PTPN21, RAB6C, RAD1, RASSF1, RB1, RBM17, RCC1, REG1A, RELA, RHOA, RHOB, RHOC, RPN2, RXRA, RXRB, SEC61A1, SGK, SHC1, SIRT1, SLC1A3, SLC35B1, SOD1, SRC, STAT1, STAT3, STK10, STK11, STMN1, TBCC, TBCD, TBCE, TFF1, TNFRSF10A, TNFRSF10B, TOP3B, TP53BP1, TSPAN4, TUBA3, TUBA6, TUBB, TUBB2C, TUBD1, UFM1, VEGF, VEGFB, VEGFC, VHL, XIST, ZW10, and ZWILCH; using the expression level to determine a likelihood of a beneficial response to a treatment including a taxane, wherein expression of DDR1, ZW10, RELA, BAX, RHOB, TSPAN4, BBC3, SHC1, CAPZA1, STK10, TBCC, EIF4E2, MCL1, RASSF1, VEGF, SLC1A3, DICER1, ILK, FAS, RAB6C, ESR1, MRE11A, APOE, BAK1, UFM1, AKT2, SIRT1, BCL2L13, ACTR2, LIMK2, HDAC6, RPN2, PLD3, RHOA, MAPK14, ECGF1, MAPRE1, HSPA1B, GATA3, PPP2CA, ABCD1, MAD2L1BP, VHL, GCLC, ACTB, BCL2L11, PRDX1, LILRB1, GNS, CHFR, CD68, LIMK1, GADD45B, VEGFB, APRT, MAP2K3, MGC52057, MAPK3, APC, RAD1, COL6A3, RXRB, CCT3, ABCC3, GPX1, TUBB2C, HSPA1A, AKT1, TUBA6, TOP3B, CSNK1D, SOD1, BUB3, MAP4, NFKB1, SEC61A1, MAD1L1, PRKCH, RXRA, PLAU, CD63, CD14, RHOC, STAT1, NPC2, NME6, PDGFRB, MGMT1, GBP1, ERCC1, RCC1, FUS, TUBA3, CHEK2, APOC1, ABCC10, SRC, TUBB, FLAD1, MAD2L2, LAPTM4B, REG1A, PRKCD, CST7, IGFBP2, FYN, KDR, STMN1, RBM17, TP53BP1, CD247, ABCA9, NTSR2, FOS, TNFRSF10A, MSH3, PTEN, GBP2, STK11, ERBB4, TFF1, ABCC1, IL7, CDC25B, TUBD1, BIRC4, ACTR3, SLC35B1, COL1A1, FOXA1, DUSP1, CXCR4, IL2RA, GGPS1, KNS2, RB1, BCL2L1, XIST, BIRC3, BID, BCL2, STAT3, PECAM1, DIABLO, CYBA, TBCE, CYP1B1, APEX1, TBCD, HRAS, TNFRSF10B, ELP3, PIK3C2A, HSPA5, VEGFC, MMP11, SGK, CTSD, BAD, PTPN21, HSPA9B, or PMS1 is positively correlated with increased likelihood of a beneficial response to a treatment including a taxane, and wherein expression of CHGA, ZWILCH, or CRABP1 is negatively correlated with an increased likelihood of a beneficial response to a treatment including a taxane; and generating a report including information based on the likelihood of a beneficial response to chemotherapy including a taxane.

The methods can further involve using a gene expression level to determine a likelihood of a beneficial response to a treatment including a cyclophosphamide, wherein expression of LILRB1, PRKCH, STAT1, GBP1, CD247, IL7, IL2RA, BIRC3, or CRABP1 is positively correlated with increased likelihood of a beneficial response to a treatment including a cyclophosphamide, and wherein expression of DDR1, ZW10, RELA, BAX, RHOB, TSPAN4, BBC3, SHC1, CAPZA1, STK10, TBCC, EIF4E2, MCL1, RASSF1, VEGF, DICER1, ILK, FAS, RAB6C, ESR1, MRE11A, APOE, BAK1, UFM1, AKT2, SIRT1, BCL2L13, ACTR2, LIMK2, HDAC6, RPN2, PLD3, CHGA, RHOA, MAPK14, ECGF1, MAPRE1, HSPA1B, GATA3, PPP2CA, ABCD1, MAD2L1BP, VHL, GCLC, ACTB, BCL2L11, PRDX1, GNS, CHFR, CD68, LIMK1, GADD45B, VEGFB, APRT, MAP2K3, MGC52057, MAPK3, APC, RAD1, COL6A3, RXRB, CCT3, ABCC3, GPX1, TUBB2C, HSPA1A, AKT1, TUBA6, TOP3B, CSNK1D, SOD1, BUB3, MAP4, NFKB1, SEC61A1, MAD1L1, RXRA, PLAU, CD63, CD14, RHOC, NPC2, NME6, PDGFRB, MGMT1, ERCC1, RCC1, FUS, TUBA3, CHEK2, APOC1, ABCC10, SRC, TUBB, FLAD1, MAD2L2, LAPTM4B, REG1A, PRKCD, CST7, IGFBP2, FYN, KDR, STMN1, ZWILCH, RBM17, TP53BP1, ABCA9, NTSR2, FOS, TNFRSF10A, MSH3, PTEN, GBP2, STK11, ERBB4, TFF1, ABCC1, CDC25B, TUBD1, BIRC4, ACTR3, SLC35B1, COL1A1, FOXA1, DUSP1, CXCR4, GGPS1, KNS2, RB1, BCL2L1, XIST, BID, BCL2, STAT3, PECAM1, DIABLO, CYBA, TBCE, CYP1B1, APEX1, TBCD, HRAS, TNFRSF10B, ELP3, PIK3C2A, HSPA5, VEGFC, MMP11, SGK, CTSD, BAD, PTPN21, HSPA9B, or PMS1 is negatively correlated with an increased likelihood of a beneficial response to a treatment including a cyclophosphamide, and wherein the report includes information based on the likelihood of a beneficial response to chemotherapy including a cyclophosphamide.

The chemotherapy can include an anthracycline. The anthracycline can be doxorubicin. Where the chemotherapy is a taxane, the taxane can be docetaxel.

The methods can accomplish measuring of the gene expression level by quantitative PCR. The methods can accomplish measuring of the gene expression level by detection of an intron-based sequence of an RNA transcript of the gene, wherein the expression of which correlates with the expression of a corresponding exon sequence.

The tumor sample can be a formalin-fixed and paraffin-embedded (FPE) or a frozen tumor section.

The methods of the present disclosure include methods of predicting whether a hormone receptor (HR) negative cancer patient will exhibit a beneficial response to chemotherapy, where the methods involve measuring an expression level of a gene, or its expression product, in a tumor sample obtained from the patient, wherein the gene is selected from the group consisting of CD247, TYMS, IGF1R, ACTG2, CCND1, CAPZA1, CHEK2, STMN1, and ZWILCH; using the expression level to determine a likelihood of a beneficial response to a treatment including a taxane, wherein expression of CD247, TYMS, IGF1R, ACTG2, CAPZA1, CHEK2, STMN1, or ZWILCH is positively correlated with increased likelihood of a beneficial response to a treatment including a taxane, and wherein expression of CCND1 is negatively correlated with an increased likelihood of a beneficial response to a treatment including a taxane; and generating a report including information based on the likelihood of a beneficial response to chemotherapy including a taxane.

The methods can further include a gene expression level to determine a likelihood of a beneficial response to a treatment including a cyclophosphamide, wherein expression of CD247, CCND1, or CAPZA1 is positively correlated with increased likelihood of a beneficial response to a treatment including a cyclophosphamide, and wherein expression of TYMS, IGF1R, ACTG2, CHEK2, STMN1, or ZWILCH is negatively correlated with an increased likelihood of a beneficial response to a treatment including a cyclophosphamide, and wherein the report includes information based on the likelihood of a beneficial response to chemotherapy including a cyclophosphamide.

The chemotherapy can include an anthracycline. The anthracycline can be doxorubicin. Where the chemotherapy is a taxane, the taxane can be docetaxel.

The methods can accomplish measuring of the gene expression level by quantitative PCR. The methods can accomplish measuring of the gene expression level by detection of an intron-based sequence of an RNA transcript of the gene, wherein the expression of which correlates with the expression of a corresponding exon sequence.

The tumor sample can be a formalin-fixed and paraffin-embedded (FPE) or a frozen tumor section.

The methods of the present disclosure include methods of predicting whether a cancer patient will exhibit a beneficial response to chemotherapy, where the methods involve measuring an expression level of a gene, or its expression product, in a tumor sample obtained from the patient, wherein the gene is selected from the group consisting of ABCC1, ABCC10, ABCC5, ACTB, ACTR2, APEX1, APOC1, APRT, BAK1, BAX, BBC3, BCL2L13, BID, BUB1, BUB3, CAPZA1, CCT3, CD247, CD68, CDCA8, CENPA, CENPF, CHEK2, CHFR, CST7, CXCR4, DDR1, DICER1, EIF4E2, GADD45B, GBP1, HDAC6, HSPA1A, HSPA1B, HSPA1L, 1L2RA, IL7, ILK, KALPHA1, KIF22, LILRB1, LIMK2, MAD2L1, MAPRE1, MCL1, MRE11A, NEK2, NTSR2, PHB, PLD3, RAD1, RALBP1, RHOA, RPN2, SHC1, SLC1A3, SRC, STAT1, STK10, STMN1, TBCC, TOP3B, TPX2, TSPAN4, TUBA3, TUBA6, TUBB, TUBB2C, TUBB3, TYMS, VEGF, VHL, WNT5A, ZW10, ZWILCH, and ZWINT; using the expression level to determine a likelihood of a beneficial response to a treatment including a taxane, wherein expression of SLC1A3, TBCC, EIF4E2, TUBB, TSPAN4, VHL, BAX, CD247, CAPZA1, STMN1, ABCC1, ZW10, HSPA1B, MAPRE1, PLD3, APRT, BAK1, CST7, SHC1, ZWILCH, SRC, GADD45B, LIMK2, CHEK2, RAD1, MRE11A, DDR1, STK10, LILRB1, BBC3, BUB3, TOP3B, RPN2, ILK, GBP1, TUBB3, NTSR2, BID, BCL2L13, ABCC5, HDAC6, CD68, DICER1, RHOA, CCT3, ACTR2, WNT5A, HSPA1L, APOC1, APEX1, KALPHA1, ABCC10, PHB, TUBB2C, RALBP1, MCL1, HSPA1A, 1L2RA, TUBA3, ACTB, KIF22, CXCR4, STAT1, IL7, or CHFR is positively correlated with increased likelihood of a beneficial response to a treatment including a taxane, and wherein expression of CENPA, CDCA8, TPX2, NEK2, TYMS, ZWINT, VEGF, BUB1, MAD2L1, or CENPF is negatively correlated with an increased likelihood of a beneficial response to a treatment including a taxane; and generating a report including information based on the likelihood of a beneficial response to chemotherapy including a taxane.

The methods can further include using a gene expression level to determine a likelihood of a beneficial response to a treatment including a cyclophosphamide, wherein expression of SLC1A3, TSPAN4, BAX, CD247, CAPZA1, ZW10, CST7, SHC1, GADD45B, MRE11A, STK10, LILRB1, BBC3, BUB3, ILK, GBP1, BCL2L13, CD68, DICER1, RHOA, ACTR2, WNT5A, HSPA1L, APEX1, MCL1, IL2RA, ACTB, STAT1, IL7, or CHFR is positively correlated with increased likelihood of a beneficial response to a treatment including a cyclophosphamide, and wherein expression of TBCC, EIF4E2, TUBB, VHL, STMN1, ABCC1, HSPA1B, MAPRE1, APRT, BAK1, TUBA6, ZWILCH, SRC, LIMK2, CENPA, CHEK2, RAD1, DDR1, CDCA8, TOP3B, RPN2, TUBB3, NTSR2, BID, TPX2, ABCC5, HDAC6, NEK2, TYMS, CCT3, ZWINT, KALPHA1, ABCC10, PHB, TUBB2C, RALBP1, VEGF, HSPA1A, BUB1, MAD2L1, CENPF, TUBA3, KIF22, or CXCR4 is negatively correlated with an increased likelihood of a beneficial response to a treatment including a cyclophosphamide, and wherein the report includes information based on the likelihood of a beneficial response to chemotherapy including a cyclophosphamide.

The chemotherapy can include an anthracycline. The anthracycline can be doxorubicin. Where the chemotherapy is a taxane, the taxane can be docetaxel.

The methods can accomplish measuring of the gene expression level by quantitative PCR. The methods can accomplish measuring of the gene expression level by detection of an intron-based sequence of an RNA transcript of the gene, wherein the expression of which correlates with the expression of a corresponding exon sequence.

The tumor sample can be a formalin-fixed and paraffin-embedded (FPE) or a frozen tumor section.

The present disclosure also provides kits containing one or more (1) extraction buffer/reagents and protocol; (2) reverse transcription buffer/reagents and protocol; and (3) qPCR buffer/reagents and protocol, suitable for performing the method disclosed herein. Also contemplated are arrays having bound polynucleotides that specifically hybridize to one or more genes used in the methods disclosed herein, as well as arrays having bound one or more antibodies that specifically bind a polypeptides expressed by a gene used in the methods disclosed herein.

Various aspects and embodiments will be apparent from the following discussion, including the Examples. Such additional embodiments, without limitation, include any and all of the ESR1 gene combinations discussed and/or specifically listed in Example 2.

### BRIEF DESCRIPTION OF THE DRAWINGS

FIG. 1 is a set of graphs showing the relationship between normalized expression (represented by “Cₜ”) of the indicated gene (gene name provided at top of each graph) and 5-year recurrence rate (RR) of breast cancer in a treatment group receiving anthracycline and a cyclophosphamide (AC prediction curve; smooth line) and the relationship between expression of the indicated gene and RR in a treatment group receiving anthracycline and a taxane (AT prediction curve; hatched line). A horizontal dashed line in each graph represents the overall (i.e., not gene expression-specific) 5-year RR in the study population who were randomized to treatment with either AC or AT. In FIG. 1 the patients were included without regard to hormone receptor expression status of the tumor.

FIG. 2 is a set of graphs showing the relationship between normalized expression (represented by “Cₜ”) of the indicated gene (gene name provided at top of each graph) and 5-year recurrence rate (RR) of breast cancer in a treatment group receiving anthracycline and a cyclophosphamide (AC prediction curve; smooth line) and the relationship between expression of the indicated gene and RR in a treatment group receiving anthracycline and a taxane (AT prediction curve; hatched line), where the patients in the treatment groups had hormone receptor positive (HR⁺) breast cancer. A horizontal dashed line in each graph represents the overall (i.e., not gene expression-specific) 5-year RR in patients in the study population having HR+breast cancer who were randomized to treatment with either AC or AT.

FIG. 3 is a set of graphs showing the relationship between normalized expression (represented by “Cₜ”) of the indicated gene (gene name provided at top of each graph) and 5-year recurrence rate (RR) of breast cancer in a treatment group receiving anthracycline and a cyclophosphamide (AC prediction curve; smooth line) and the relationship between expression of the indicated gene and RR in a treatment group receiving anthracycline and a taxane (AT prediction curve; hatched line), where the patients in the treatment groups had hormone receptor positive (HR⁺) breast cancer and an Oncotype Dx Recurrence Score of greater than 18. A horizontal dashed line in each graph represents the overall (i.e., not gene expression-specific) 5-year RR in patients in the study having HR+breast cancer and an Oncotype Dx Recurrence Score greater than 18 who were randomized to treatment with either AC or AT.

FIG. 4 is a set of graphs showing the relationship between normalized expression (represented by “Cₜ”) of the indicated gene (gene name provided at top of each graph) and 5-year recurrence rate (RR) of breast cancer in a treatment group receiving an anthracycline and a cyclophosphamide (AC prediction curve; smooth line) and the relationship between expression of the indicated gene and RR in a treatment group receiving anthracycline and a taxane (AT prediction curve; hatched line), where the patients in the treatment groups had hormone receptor negative (HR⁻) breast. A horizontal dashed line in each graph represents the overall (i.e., not gene expression-specific) 5-year RR in patients in the study having HR⁻ breast cancer who were randomized to treatment with either AC or AT

FIG. 5 is a graph illustrating the impact of using DDR1 to select HR-positive patients for treatment with AC vs AT. The dotted line depicts the relationship between normalized expression of DDR1 and the 5-year recurrence rate (RR) of breast cancer in the AC treatment group (the AC prediction curve, also referred to as the cyclophosphamide benefit (CB) curve); the solid line depicts the relationship between normalized expression of DDR1 and the 5-year recurrence rate (RR) of breast cancer in the AT treatment group (the AT prediction curve, also referred to as the taxane benefit (TB) curve. Expression is provided on the x-axis as a normalized DDR1 expression level (relative to reference genes; log 2). The y-axis provides the risk of cancer recurrence at 5 years.

The following Appendices and Tables are provided in the specification just prior to the claims.

Appendix 1. RT-PCR probe and primer sequences

Appendix 2. RT-PCR amplicon sequences

Table 1. Differential Markers of Response Identified in Breast Cancer Patients, All Patients.

Table 2. Differential Markers of Response Identified in Breast Cancer Patients, HR-Positive Patients

Table 3. Differential Markers of Response Identified in Breast Cancer Patients, HR-Positive Patients, RS>18

Table 4. Differential Markers of Response Identified in Breast Cancer Patients, HR-Negative Patients.

Table 5. Additional genes involved in NFκB signaling

### DETAILED DESCRIPTION OF EXEMPLARY EMBODIMENTS

### Definitions

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3^{rd} ed., J. Wiley & Sons, Chichester, N.Y., 2001.

As used herein, the term “anthracycline” refers to a class of antineoplastic antibiotics that are typically derived by Streptomyces bacteria (e.g., Streptomyces peucetius or Streptomyces coeruleorubidus). Although the precise mechanism of action is unknown, anthracyclines are believed to derive their chemotherapeutic activity, at least in part, from their ability to damage DNA by intercalation, metal ion chelation, and the generation of free radicals and can inhibit enzyme activity critical to DNA function. Examples of anthracyclines include daunorubicin, doxorubicin, epirubicin, idarubicin, amrubicin, pirarubicin, valrubicin, zorubicin, caminomycin, detorubicin, esorubicin, marcellomycin, quelamycin, rodorubicin, and aclarubicin, as well as pharmaceutically active salts, acids or derivatives of any of these.

As used herein, the term “taxane” refers to a family of antimitotic/antimicrotubule agents that inhibit cancer cell growth by stopping cell division. Examples of taxanes include paclitaxel, docetaxel, larotaxel, ortataxel, tesetaxel and other related diterpene compounds that have chemotherapeutic activity as well as pharmaceutically active salts, acids or derivatives of any of these. Paclitaxel was originally derived from the Pacific yew tree. Related diterpenes are produced by plants of the genus Taxus (yews) and synthetic or semi-synthetic taxanes with chemotherapeutic activity have also been synthesized, e.g., docetaxel, and are encompassed in the term taxane.

As used herein, the term “cyclophosphasmide” refers to a cytotoxic alkylating agent of the nitrogen mustard group, including the chemotherapeutic compound N,N-bis(2-chloroethyl)-1,3,2-oxazaphosphinan-2-amine 2-oxide (also known as cytophosphane). It is a highly toxic, immunosuppressive, antineoplastic drug, used in the treatment of Hodgkin's disease, lymphoma, and certain other forms of cancer, such as leukemia and breast cancer.

A “taxane-containing treatment” (also referred to as “taxane-containing regimen” or “taxane-containing treatment regimen”) or “cyclophosphamide-containing treatment” (also referred to as “cyclophosphamide-containing regimen” or “cyclophosphamide-containing treatment regimen”) is meant to encompass therapies in which a taxane or a cyclophosphamide, respectively, is administered alone or in combination with another therapeutic regimen (e.g., another chemotherapy (e.g., anthracycline), or both). Thus, a taxane-containing treatment can include, for example, administration a taxane in combination with anthracyline, with anthracyline and cyclosphophamide, and the like.

The term “in combination with” such as when used in reference to a therapeutic regimen, refers to administration or two or more therapies over the course of a treatment regimen, where the therapies may be administered together or separately, and, where used in reference to drugs, may be administered in the same or different formulations, by the same or different routes, and in the same or different dosage form type.

The term “prognosis” is used herein to refer to the prediction of the likelihood of cancer-attributable death or progression, including recurrence, of a neoplastic disease, such as breast cancer, in a patient. The concept of prognosis is used in the context of the minimal standard of care. For example, in the context of early stage, ER+ invasive breast care, the minimal standard of care could be surgery plus adjuvant hormonal therapy.

The term “prediction” is used herein to refer to a likelihood that a patient will have a particular clinical outcome following administration of a treatment regimen, e.g., a chemotherapeutic regimen. Clinical benefit may be measured, for example, in terms of clinical outcomes such as disease recurrence, tumor shrinkage, and/or disease progression.

The term “patient” or “subject” as used herein refers to a human patient.

The term “long-term” survival is used herein to refer to survival for at least 3 years, more preferably for at least 8 years, most preferably for at least 10 years following surgery or other treatment.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth.

The term “breast cancer” is used herein to include all forms and stages of breast cancer, including, without limitation, locally advanced breast cancer, invasive breast cancer, and metastatic breast cancer.

A “tumor sample” as used herein is a sample derived from, or containing tumor cells from, a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the term “expression level” as applied to a gene refers to the normalized level of a gene product, e.g. the normalized value determined for the RNA expression level of a gene or for the polypeptide expression level of a gene.

The term “Cₜ” as used herein refers to threshold cycle, the cycle number in quantitative polymerase chain reaction (qPCR) at which the fluorescence generated within a reaction well exceeds the defined threshold, i.e. the point during the reaction at which a sufficient number of amplicons have accumulated to meet the defined threshold.

The terms “threshold” or “thresholding” refer to a procedure used to account for non-linear relationships between gene expression measurements and clinical response as well as to further reduce variation in reported patient scores. When thresholding is applied, all measurements below or above a threshold are set to that threshold value. Non-linear relationship between gene expression and outcome could be examined using smoothers or cubic splines to model gene expression in Cox PH regression on recurrence free interval or logistic regression on recurrence status. Variation in reported patient scores could be examined as a function of variability in gene expression at the limit of quantitation and/or detection for a particular gene.

The term “gene product” or “expression product” are used herein to refer to the RNA transcription products (transcripts) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The term “RNA transcript” as used herein refers to the RNA transcription products of a gene, including, for example, mRNA, an unspliced RNA, a splice variant mRNA, a microRNA, and a fragmented RNA.

Unless indicated otherwise, each gene name used herein corresponds to the Official Symbol assigned to the gene and provided by Entrez Gene (URL: http://www.ncbi.nlm.nih.gov/sites/entrez) as of the filing date of this application.

The terms “correlated” and “associated” are used interchangeably herein to refer to a strength of association between two measurements (or measured entities). The disclosure provides genes and gene subsets, the expression levels of which are associated with a particular outcome measure, such as for example between the expression level of a gene and the likelihood of beneficial response to treatment with a drug. For example, the increased expression level of a gene may be positively correlated (positively associated) with an increased likelihood of good clinical outcome for the patient, such as an increased likelihood of long-term survival without recurrence of the cancer and/or beneficial response to a chemotherapy, and the like. Such a positive correlation may be demonstrated statistically in various ways, e.g. by a low hazard ratio. In another example, the increased expression level of a gene may be negatively correlated (negatively associated) with an increased likelihood of good clinical outcome for the patient. In that case, for example, the patient may have a decreased likelihood of long-term survival without recurrence of the cancer and/or beneficial response to a chemotherapy, and the like. Such a negative correlation indicates that the patient likely has a poor prognosis or will respond poorly to a chemotherapy, and this may be demonstrated statistically in various ways, e.g., a high hazard ratio.

A “positive clinical outcome” and “beneficial response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Positive clinical response may also be expressed in terms of various measures of clinical outcome. Positive clinical outcome can also be considered in the context of an individual's outcome relative to an outcome of a population of patients having a comparable clinical diagnosis, and can be assessed using various endpoints such as an increase in the duration of Recurrence-Free interval (RFI), an increase in the time of survival as compared to Overall Survival (OS) in a population, an increase in the time of Disease-Free Survival (DFS), an increase in the duration of Distant Recurrence-Free Interval (DRFI), and the like. An increase in the likelihood of positive clinical response corresponds to a decrease in the likelihood of cancer recurrence.

The term “risk classification” means a level of risk (or likelihood) that a subject will experience a particular clinical outcome. A subject may be classified into a risk group or classified at a level of risk based on the methods of the present disclosure, e.g. high, medium, or low risk. A “risk group” is a group of subjects or individuals with a similar level of risk for a particular clinical outcome.

The term “normalized expression” with regard to a gene or an RNA transcript or other expression product (e.g., protein) is used to refer to the level of the transcript (or fragmented RNA) determined by normalization to the level of reference mRNAs, which might be all measured transcripts in the specimen or a particular reference set of mRNAs. A gene exhibits “increased expression” or “increased normalized expression” in a subpopulation of subjects when the normalized expression level of an RNA transcript (or its gene product) is higher in one clinically relevant subpopulation of patients (e.g., patients who are responsive to chemotherapy treatment) than in a related subpopulation (e.g., patients who are not responsive to said chemotherapy). In the context of an analysis of a normalized expression level of a gene in tissue obtained from an individual subject, a gene is exhibits “increased expression” when the normalized expression level of the gene trends toward or more closely approximates the normalized expression level characteristic of such a clinically relevant subpopulation of patients. Thus, for example, when the gene analyzed is a gene that shows increased expression in responsive subjects as compared to non-responsive subjects, then if the expression level of the gene in the patient sample trends toward a level of expression characteristic of a responsive subject, then the gene expression level supports a determination that the individual patient is likely to be a responder. Similarly, where the gene analyzed is a gene that is increased in expression in non-responsive patients as compared to responsive patients, then if the expression level of the gene in the patient sample trends toward a level of expression characteristic of a non-responsive subject, then the gene expression level supports a determination that the individual patient will be nonresponsive. Thus normalized expression of a given gene as disclosed herein can be described as being positively correlated with an increased likelihood of positive clinical response to chemotherapy or as being positively correlated with a decreased likelihood of a positive clinical response to chemotherapy.

The term “recurrence score” or “RS” refers to an algorithm-based indicator useful in determining the likelihood of an event of interest, such as a likelihood of cancer recurrence and/or the likelihood that a patient will respond to a treatment modality as may be assessed by cancer recurrence following therapy with the treatment modality.

The term “hormone receptor positive (HR+) tumor” means a tumor expressing either estrogen receptor (ER+) or progesterone receptor (PR+) above a certain threshold as determined by standard methods, including immunohistochemical staining of nuclei and polymerase chain reaction (PCR) in a biological sample obtained from a patient. The term “hormone receptor negative (HR−) tumor” means a tumor that does not express either estrogen receptor (ER−) or progesterone receptor (PR−) above a certain threshold. The threshold may be measured, for example, using an Allred score or gene expression. See, e.g., J. Harvey, et al., J Clin Oncol 17:1474-1481 (1999); S. Badve, et al., J Clin Oncol 26(15):2473-2481 (2008).

“Overall survival (OS)” refers to the patient remaining alive for a defined period of time, such as 1 year, 5 years, etc, e.g., from the time of diagnosis or treatment.

“Progression-free survival (PFS)” refers to the patient remaining alive, without the cancer getting worse.

“Neoadjuvant therapy” is adjunctive or adjuvant therapy given prior to the primary (main) therapy. Neoadjuvant therapy includes, for example, chemotherapy, radiation therapy, and hormone therapy. Thus, chemotherapy may be administered prior to surgery to shrink the tumor, so that surgery can be more effective, or, in the case of previously unoperable tumors, possible.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of the present invention, reference to “at least one,” “at least two,” “at least five,” etc. of the genes listed in any particular gene set means any one or any and all combinations of the genes listed.

Herein, numerical ranges or amounts prefaced by the term “about” expressly include the exact range or exact numerical amount.

### General Description

The disclosed methods are useful to facilitate treatment decisions by providing an assessment of the likelihood of clinical benefit to a treatment that includes a taxane, a treatment that includes a cyclophosphamide, or both. Because taxanes and cyclophosphamide have different mechanisms of action, it is possible that tumors of certain patients exhibit molecular pathology that makes them more likely to respond to one drug type than the other. For example, the methods disclosed herein can be used to facilitate treatment decisions by providing an assessment of the likelihood of clinical benefit to an anthracycline-based treatment that includes a taxane, an anthracycline-based treatment that includes a cyclophosphamide, or an anthracycline-based treatment that includes both a cyclophosphamide and a taxane. Accordingly, such predictive methods are useful to facilitate chemotherapy treatment decisions that are tailored to individual patients. For example, the methods disclosed herein can be used to assess whether there is clinical benefit to addition of a taxane to a chemotherapeutic regimen.

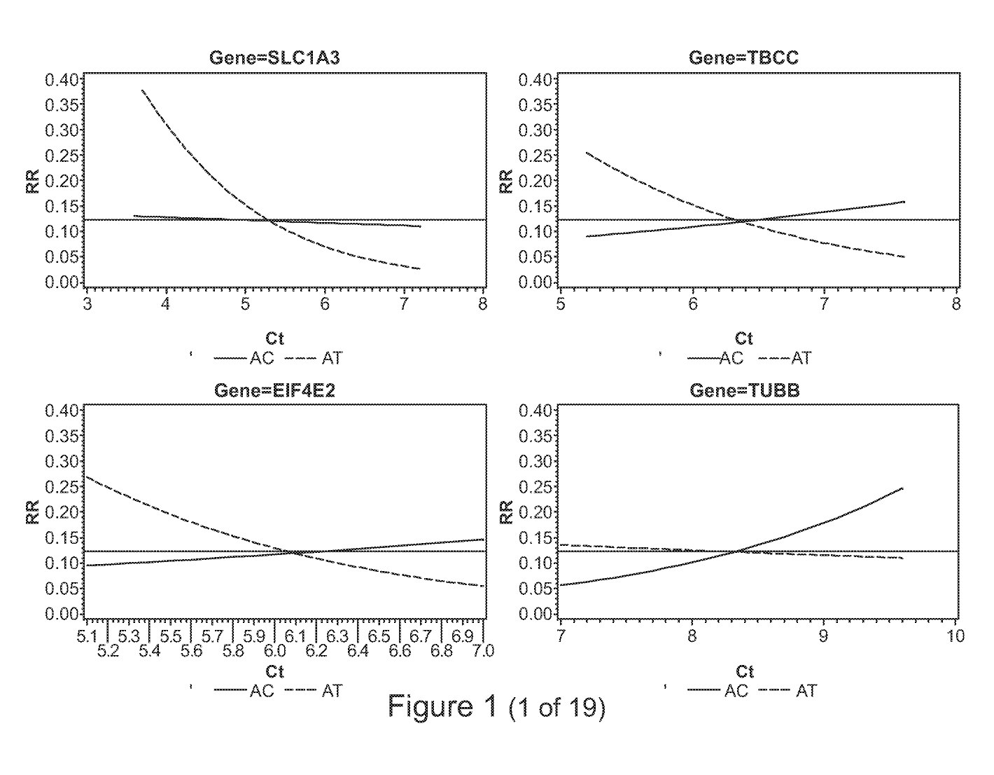

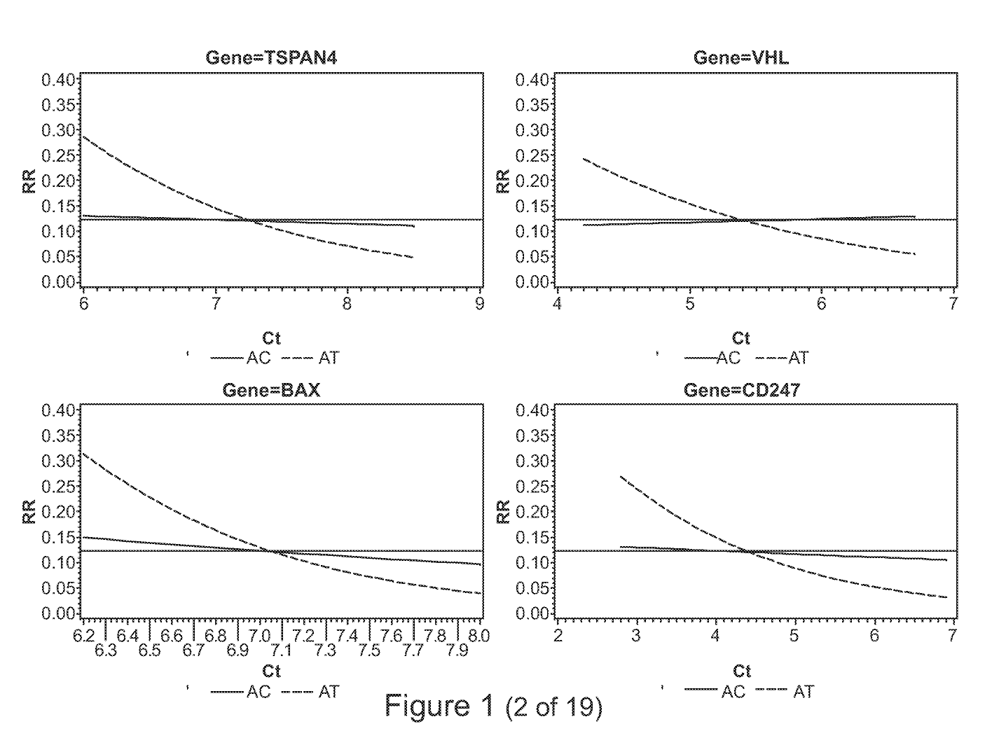

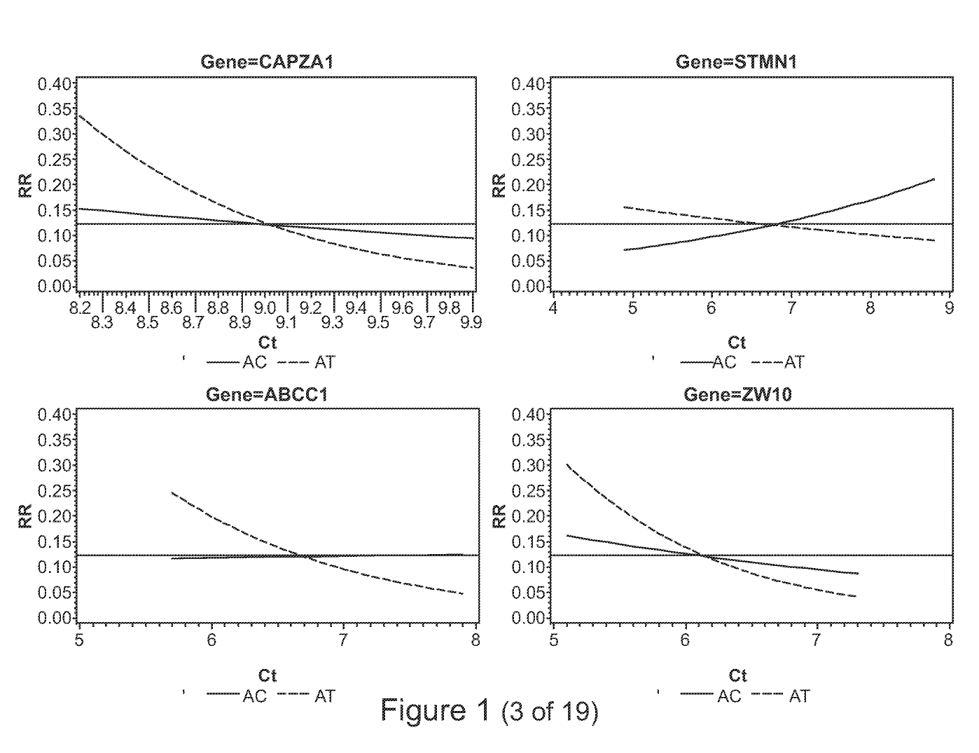

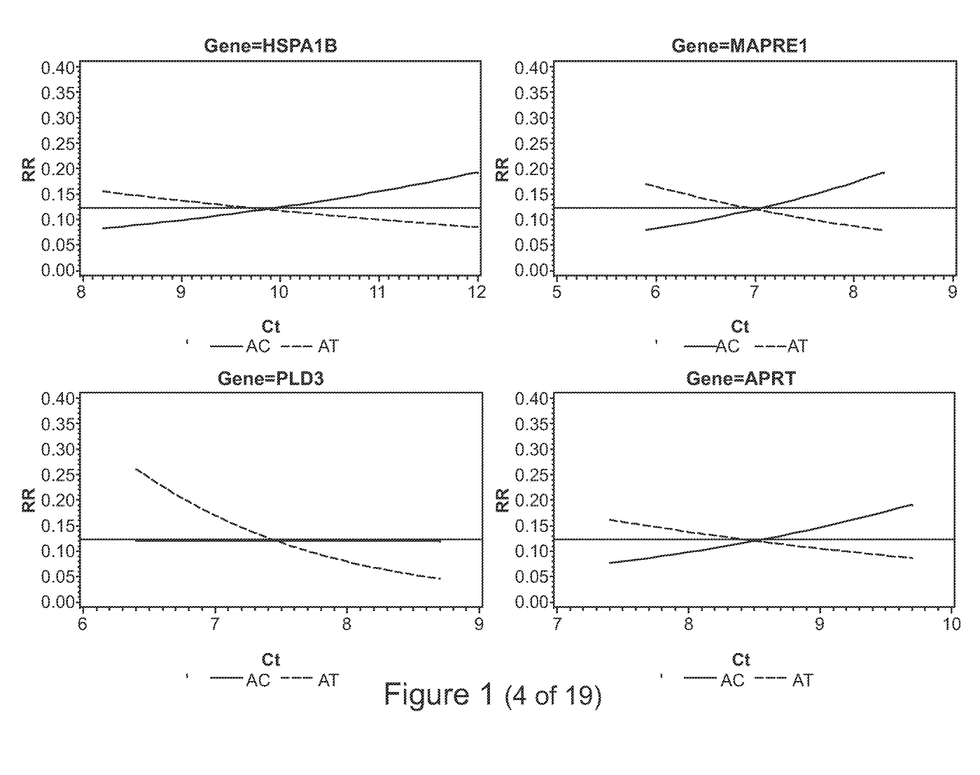

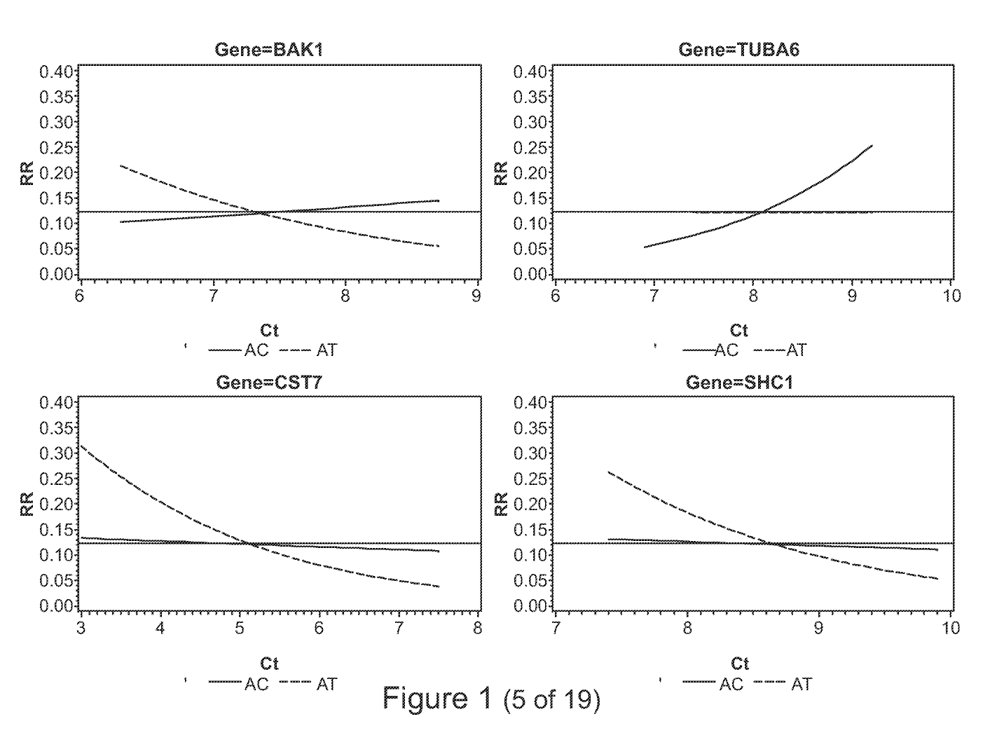

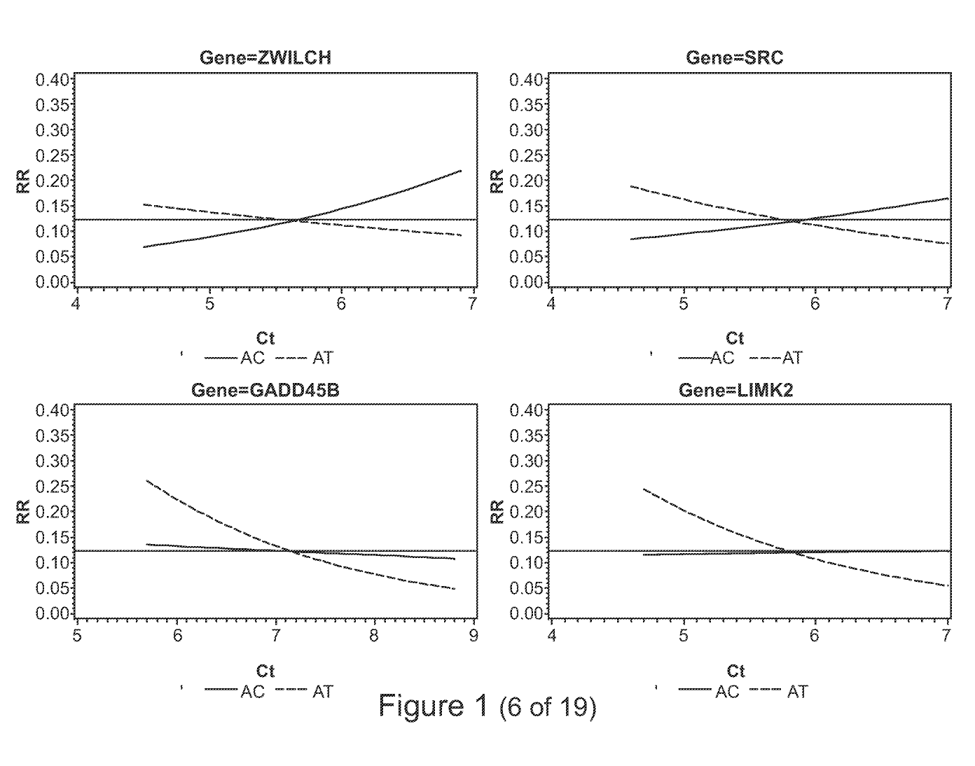

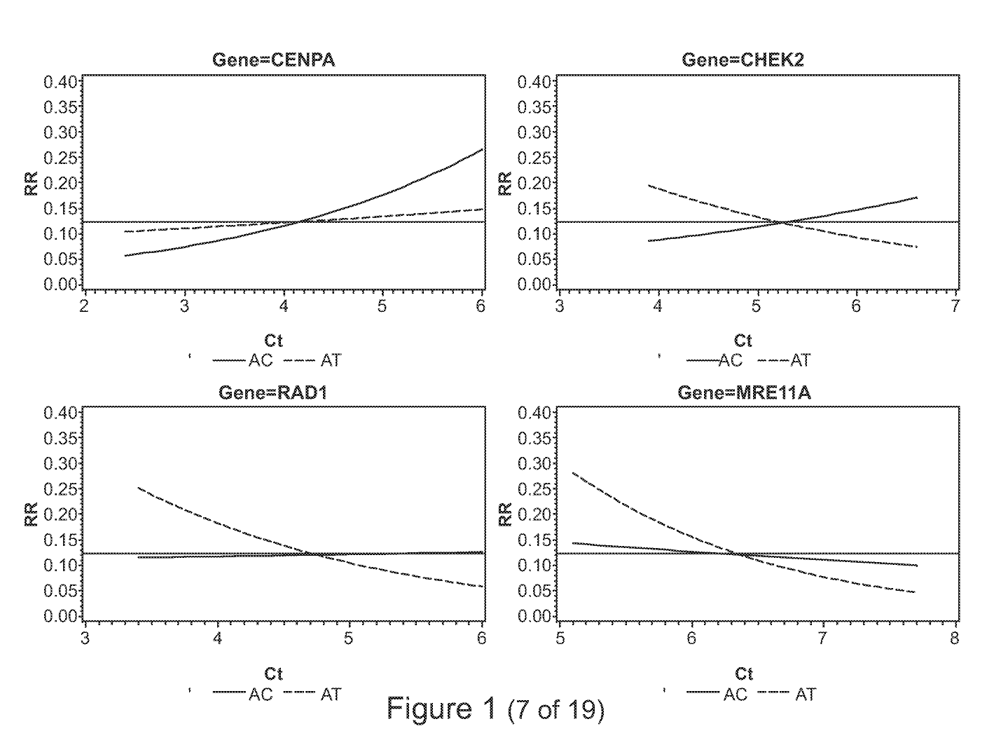

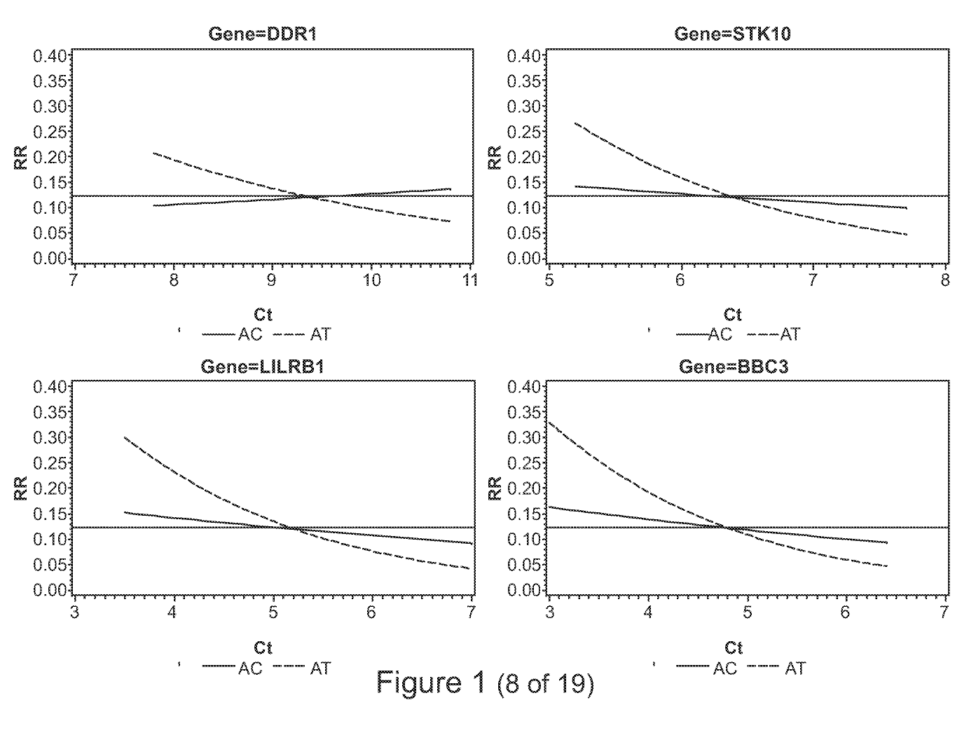

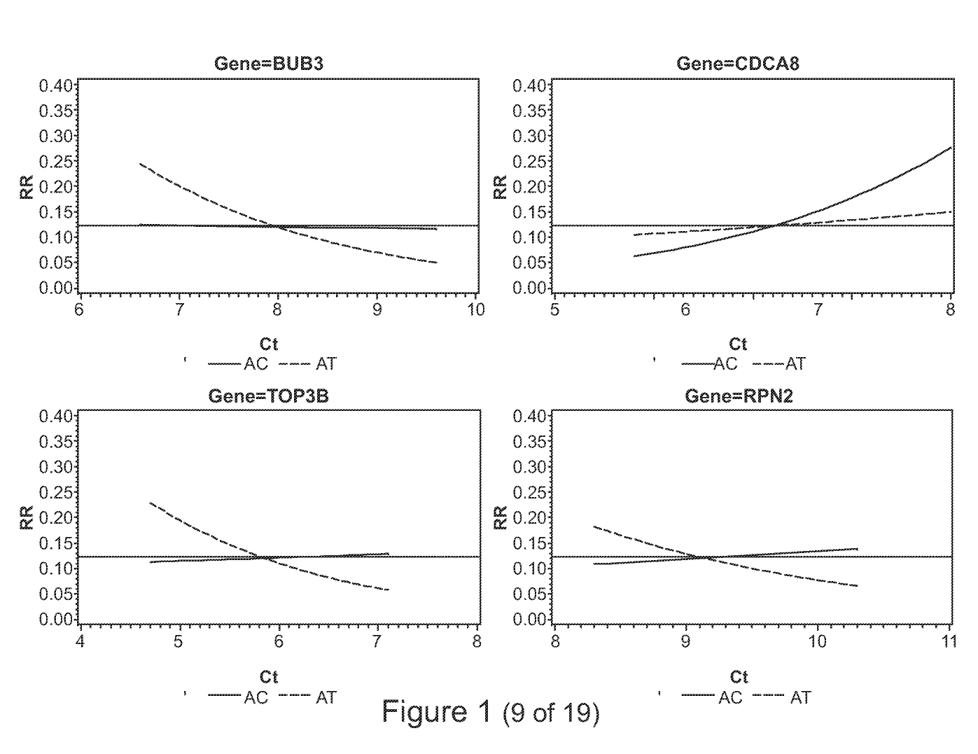

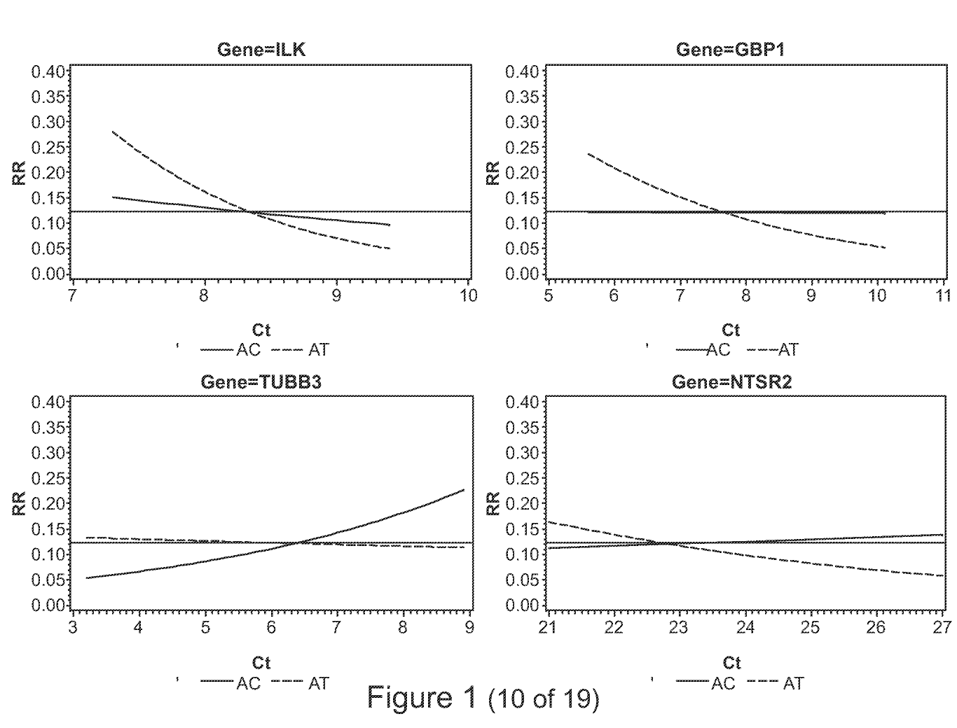

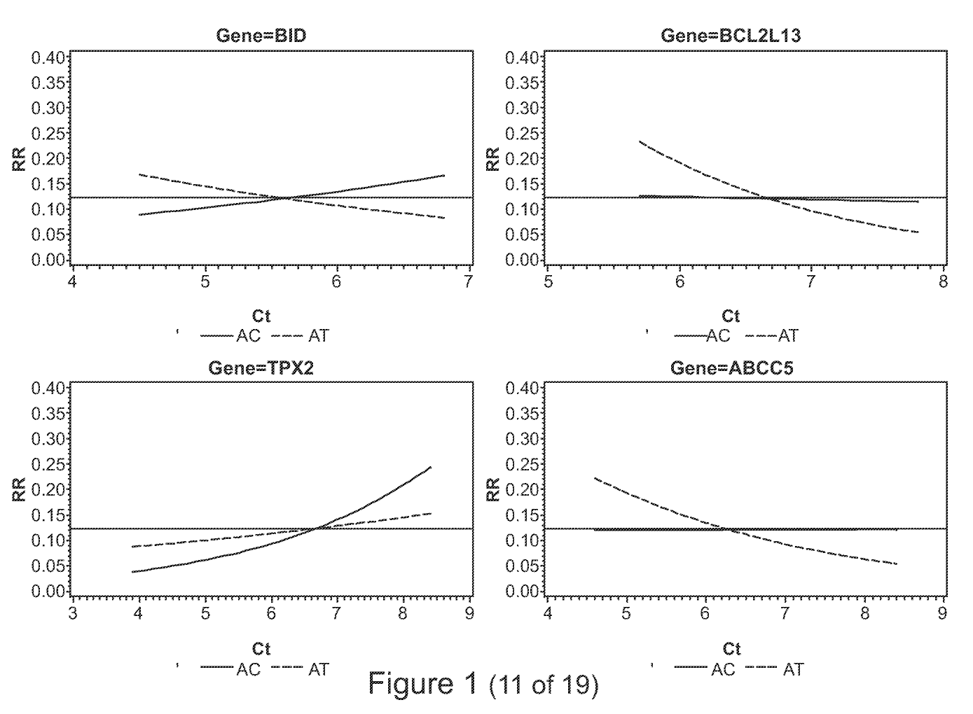

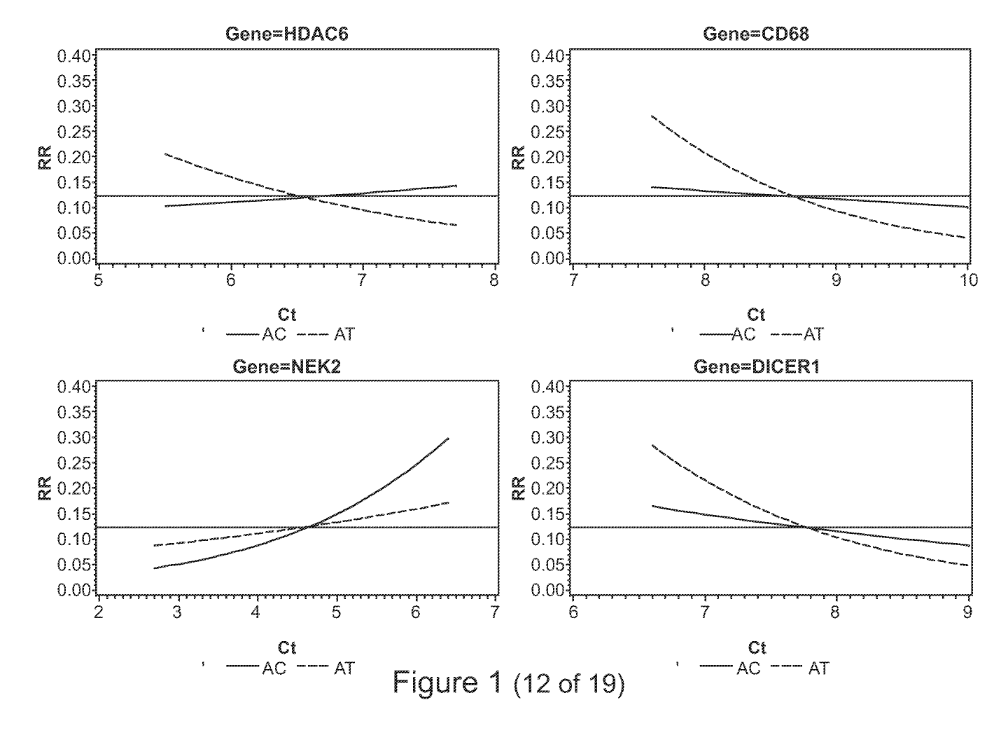

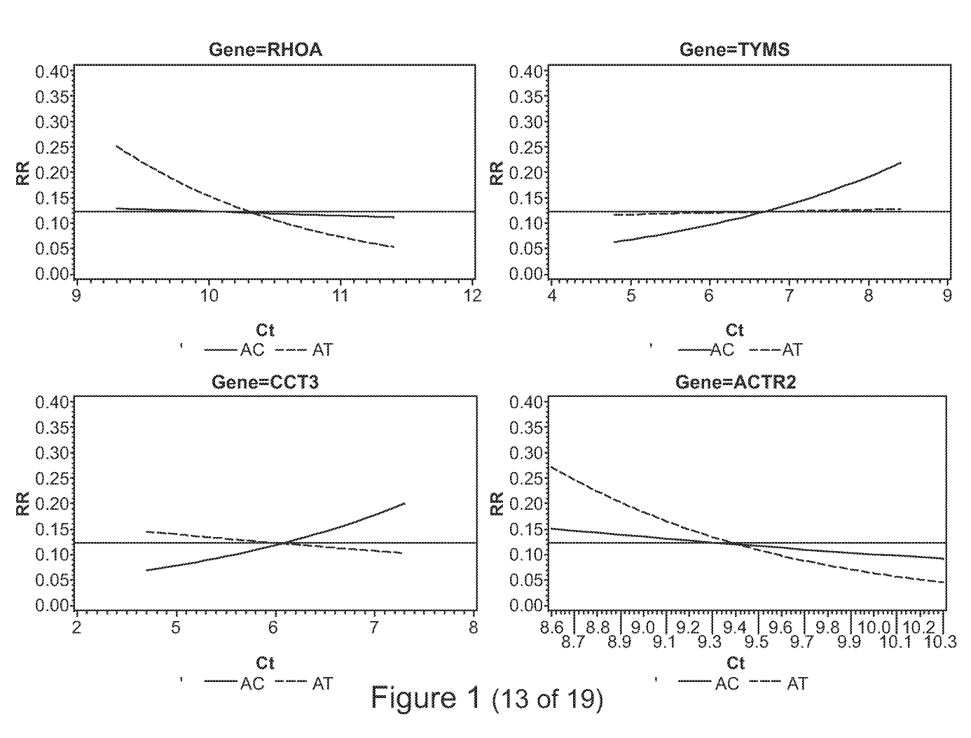

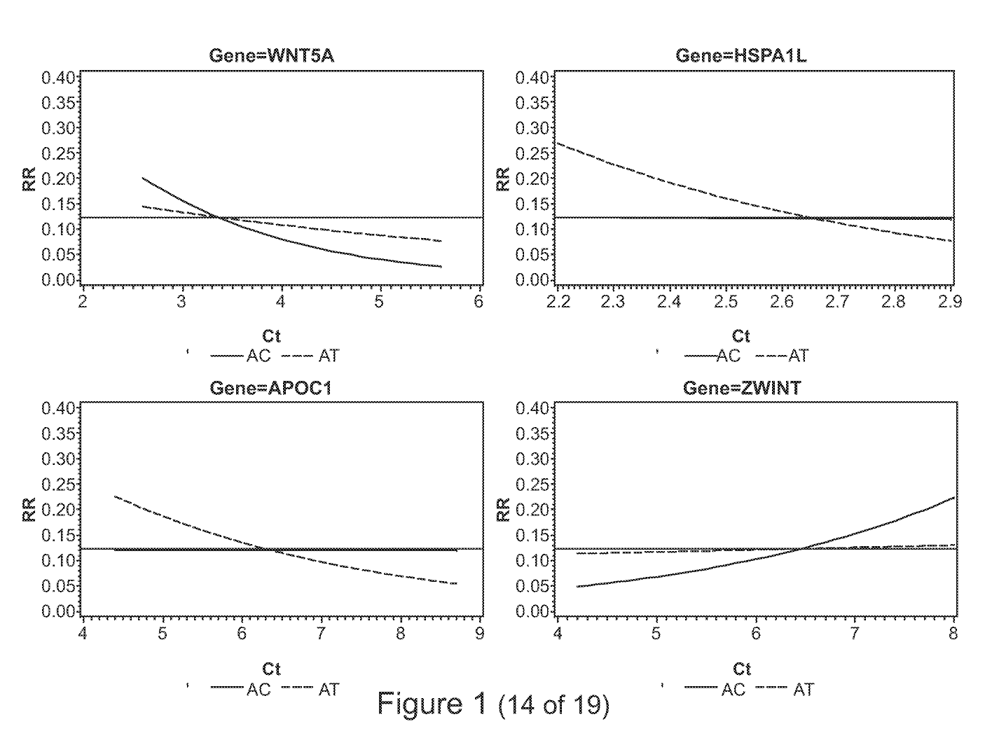

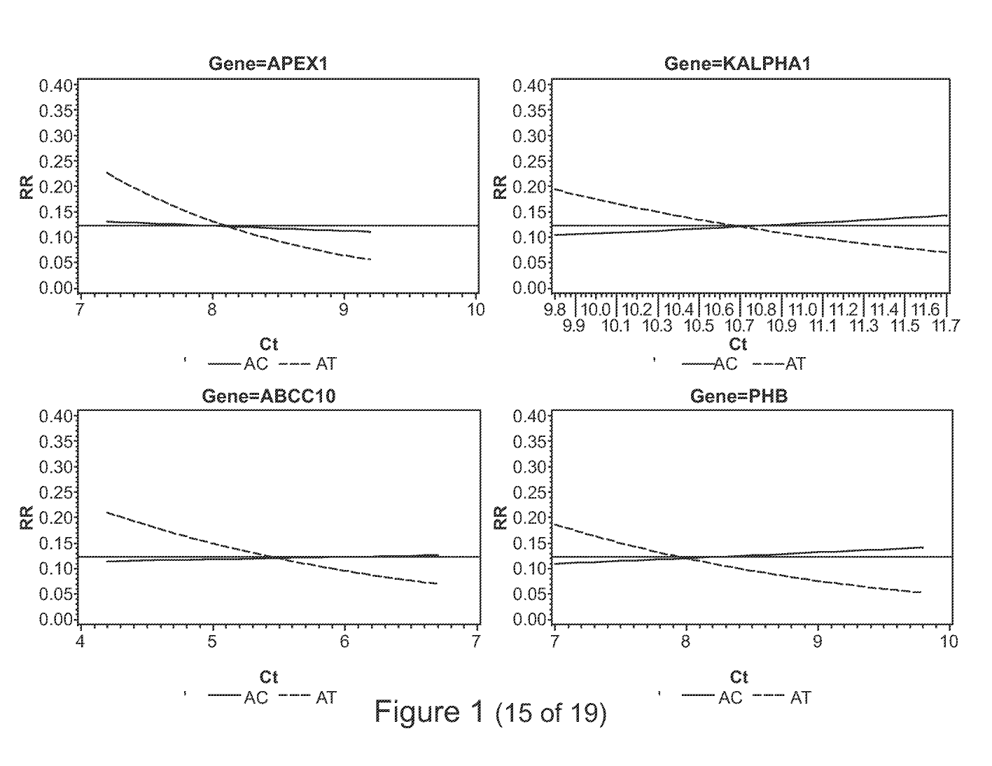

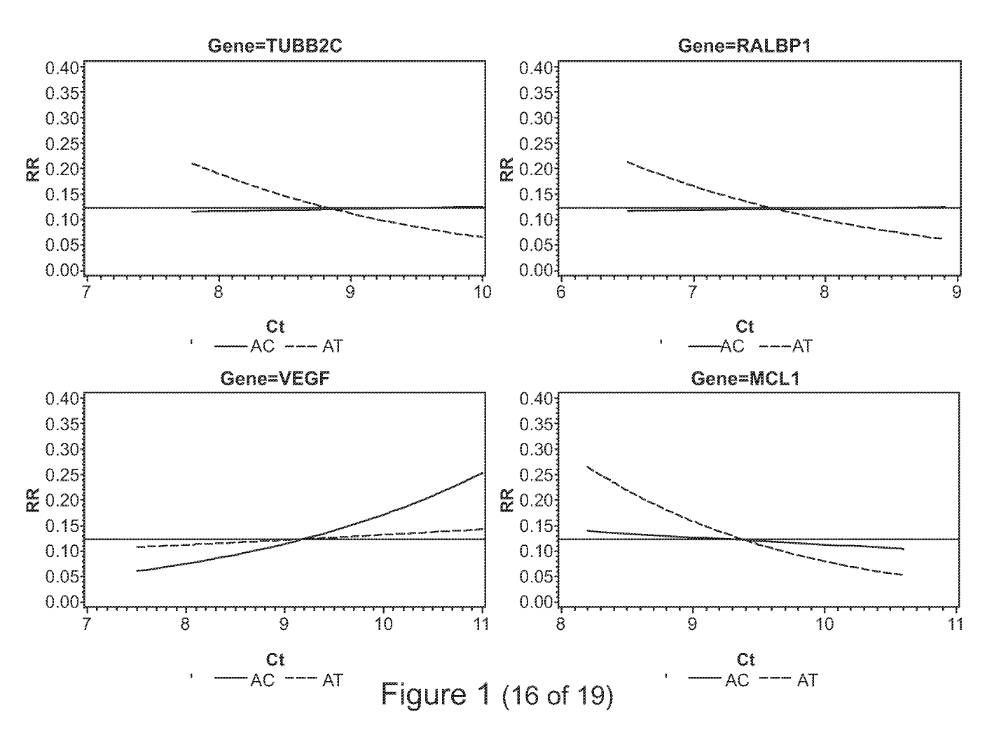

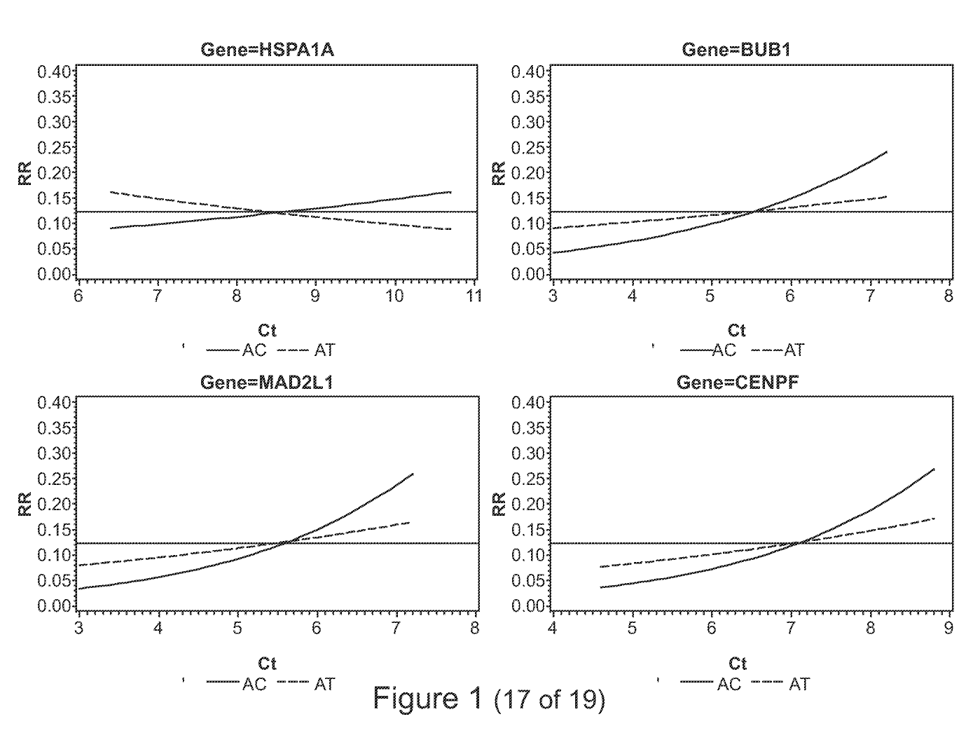

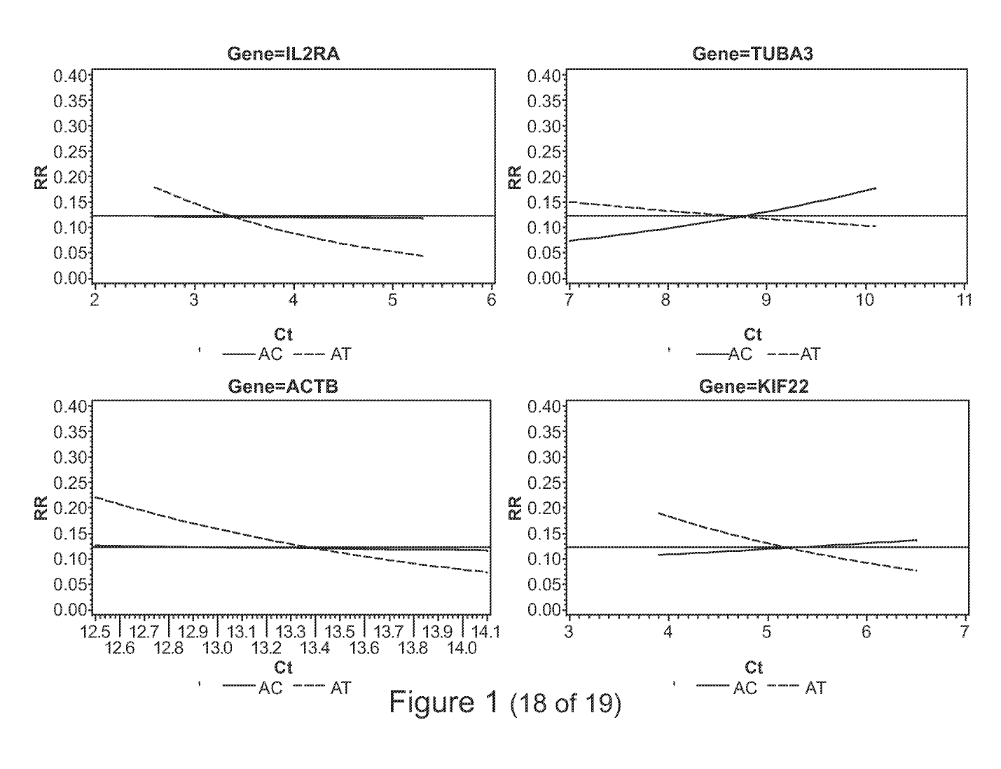

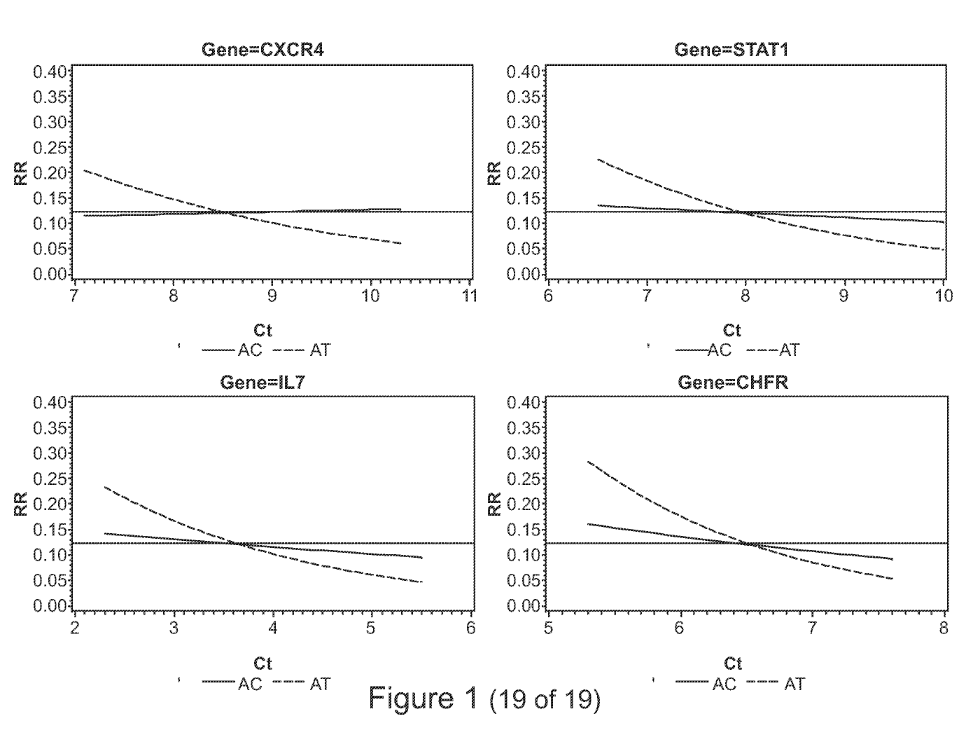

Genes for which expression is correlated either positively or negatively with increased likelihood of response to a treatment that includes a taxane, a treatment that includes a cyclophosphamide, or both are provided in FIGS. 1-4 and Tables 1-4.

The relationships between expression level of a marker gene of the present disclosure and a positive or negative correlation with likelihood of recurrence of cancer (e.g., breast cancer) following treatment with a taxane-containing regimen or a cyclophosphamide-containing regimen are exemplified in FIGS. 1-4. The hatched line in each graph represents the relationship between expression of the gene in patients treated with a taxane-containing regimen (e.g., anthracycline plus a taxane) and the 5-year recurrence rate (RR) of cancer (the taxane benefit (TB) prediction curve). The TB prediction line thus represents the correlation of expression of the gene and the likelihood of clinical benefit of a taxane in a treatment regimen. The smooth line in each graph represents the relationship between expression of the gene in patients treated with a cyclophosphamide-containing regimen (e.g., anthracycline plus cyclophosphamide) and the 5-year recurrence rate (RR) of cancer (the cyclophosphamide benefit (CB) prediction curve). The CB prediction curve thus represents the correlation of expression of the gene and the likelihood of clinical benefit of a cyclophosphamide in a treatment regimen. Because the patients in the study also received an anthracycline, the TB prediction curve and CB prediction curve can also be considered an anthracycline plus a taxane (AT) benefit prediction curve and an anthracycline plus a cyclophosphamide (AC) benefit prediction curve, respectively.

Each of the graphs in FIGS. 1-4 include a horizontal dashed line that represents the overall (i.e., not gene expression-specific) recurrence rate at 5-years in the relevant population who were randomized to treatment with AC or AT. The difference between the TB and CB prediction curves and this horizontal line depicts the extent to which clinical benefit may be improved by a gene expression-guided treatment decision.

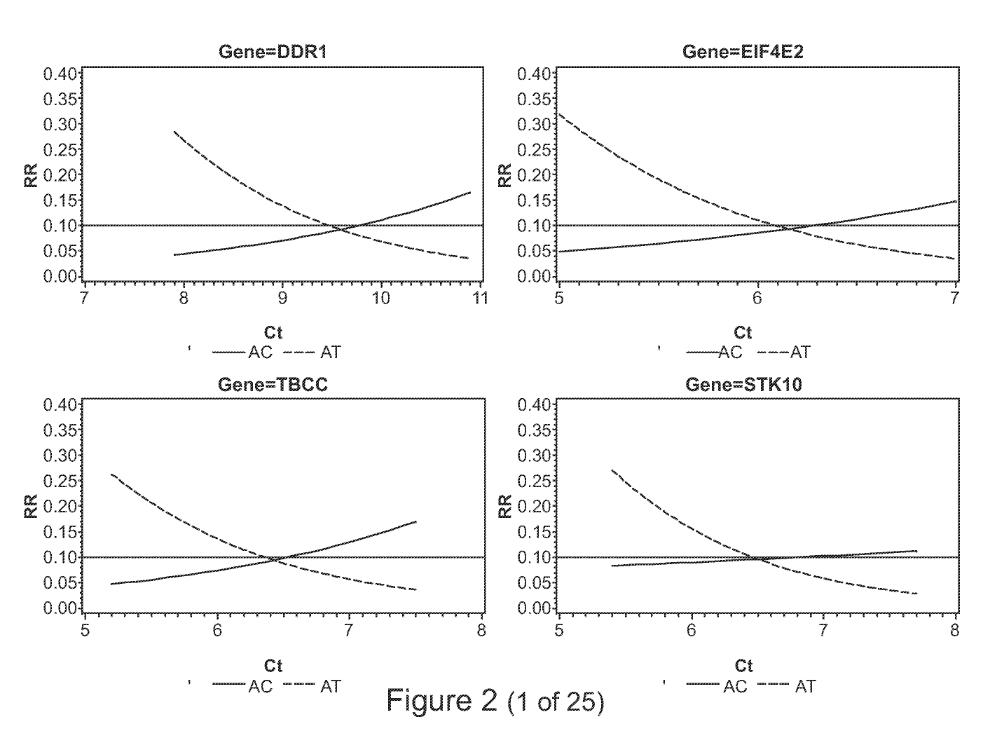

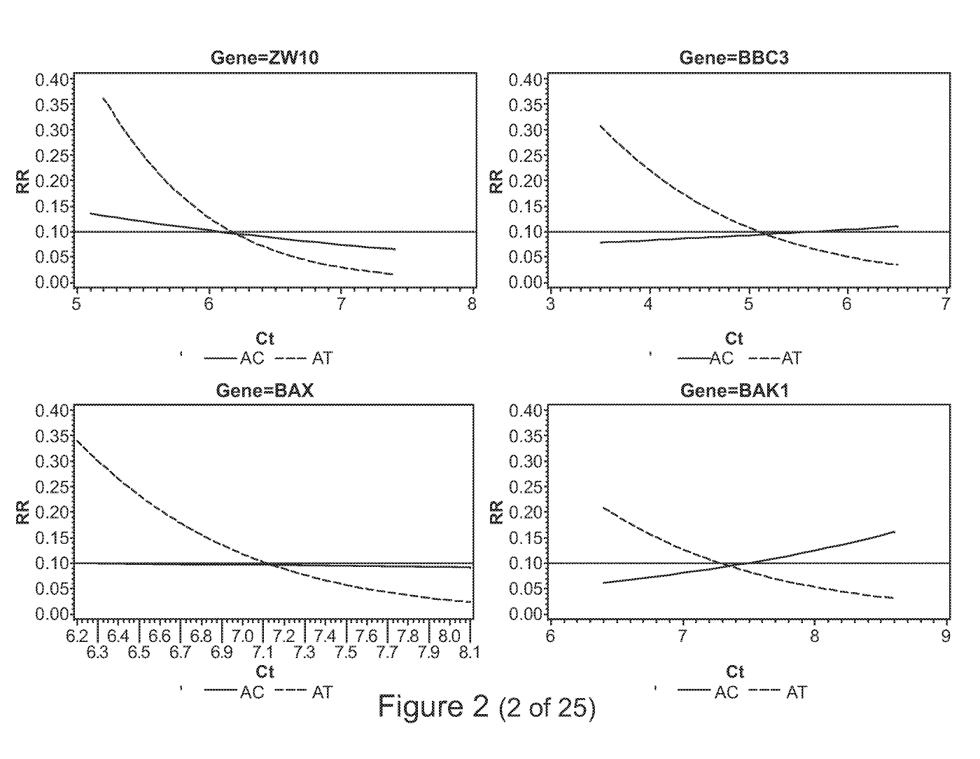

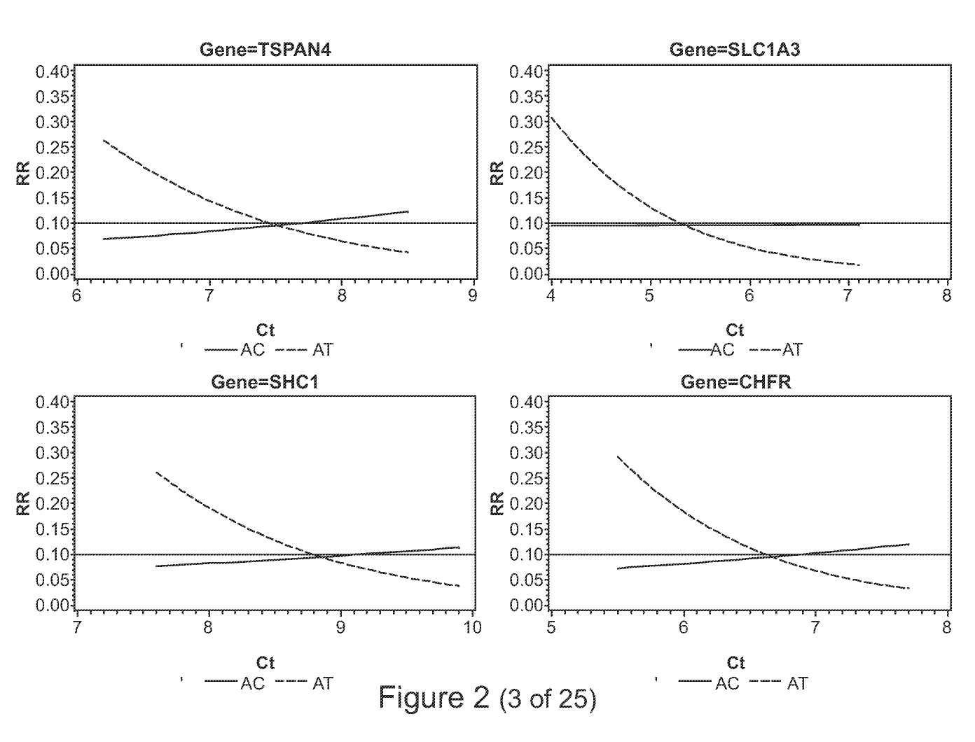

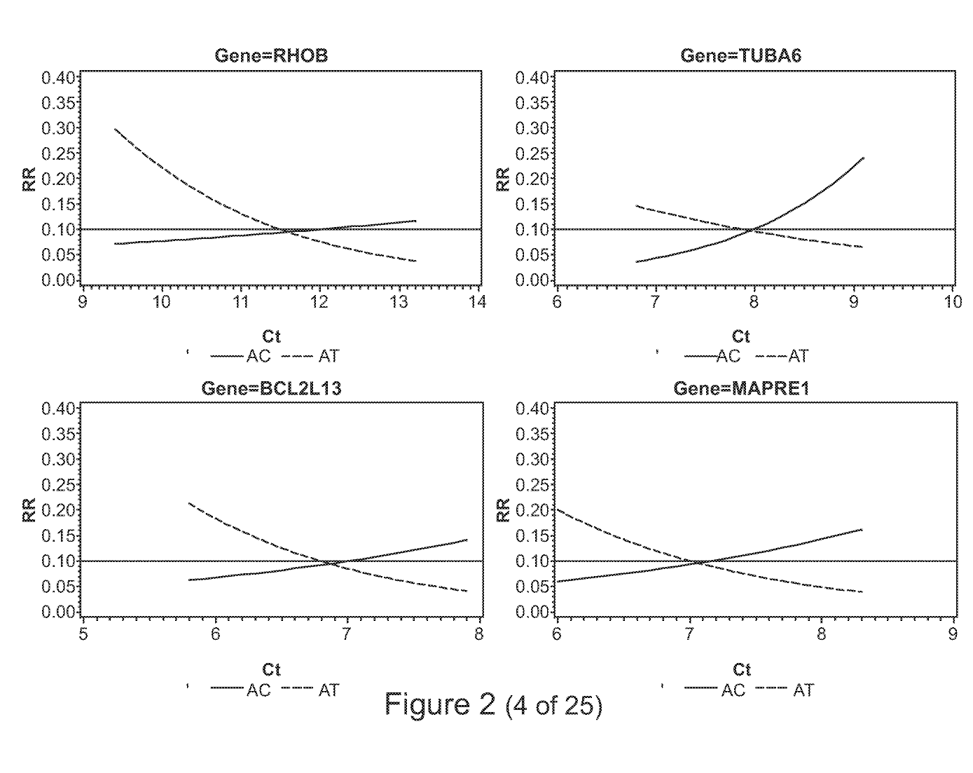

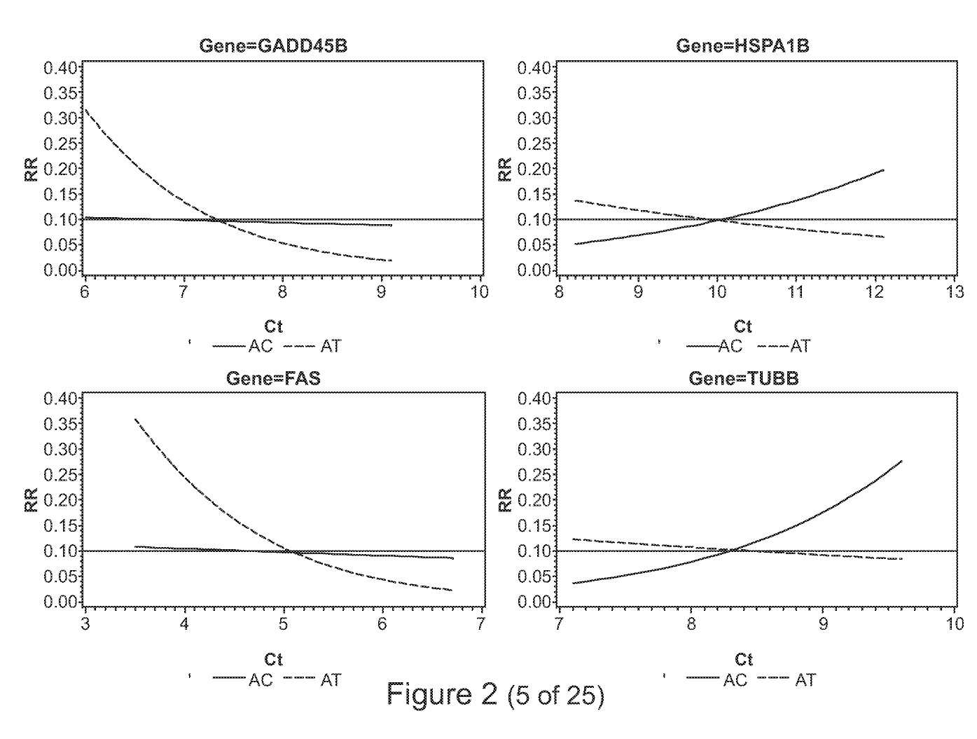

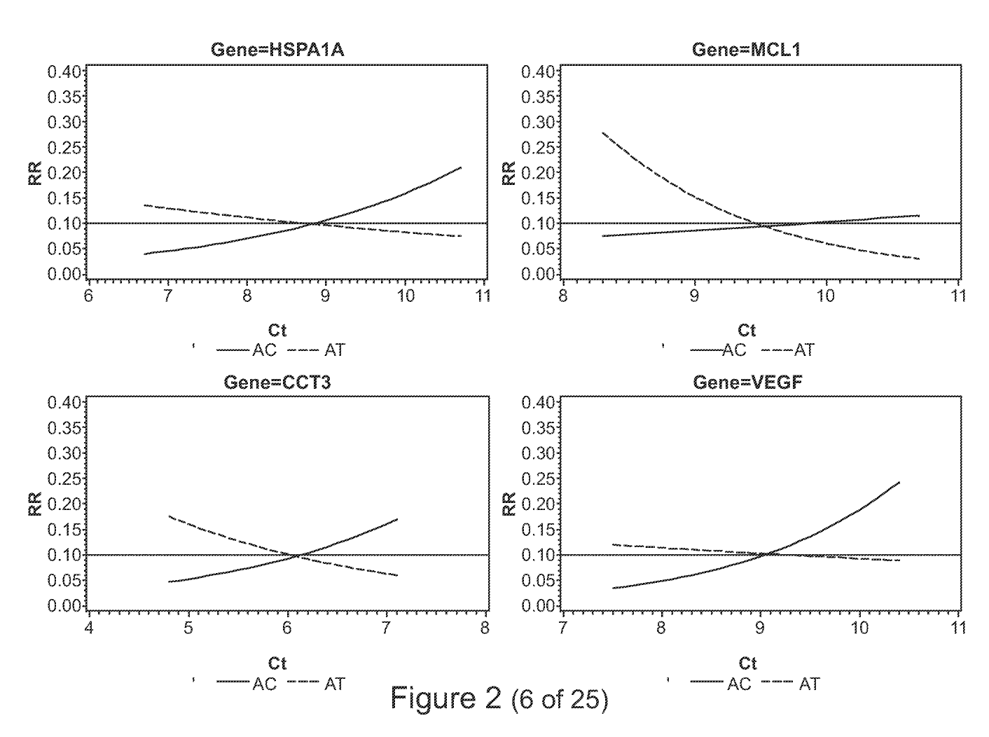

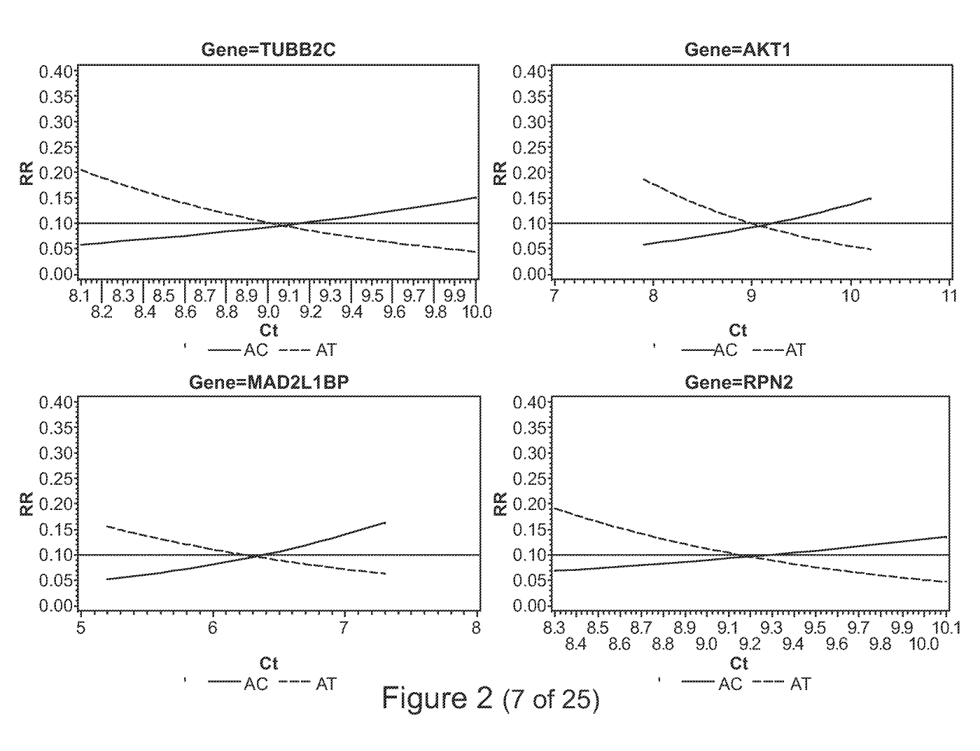

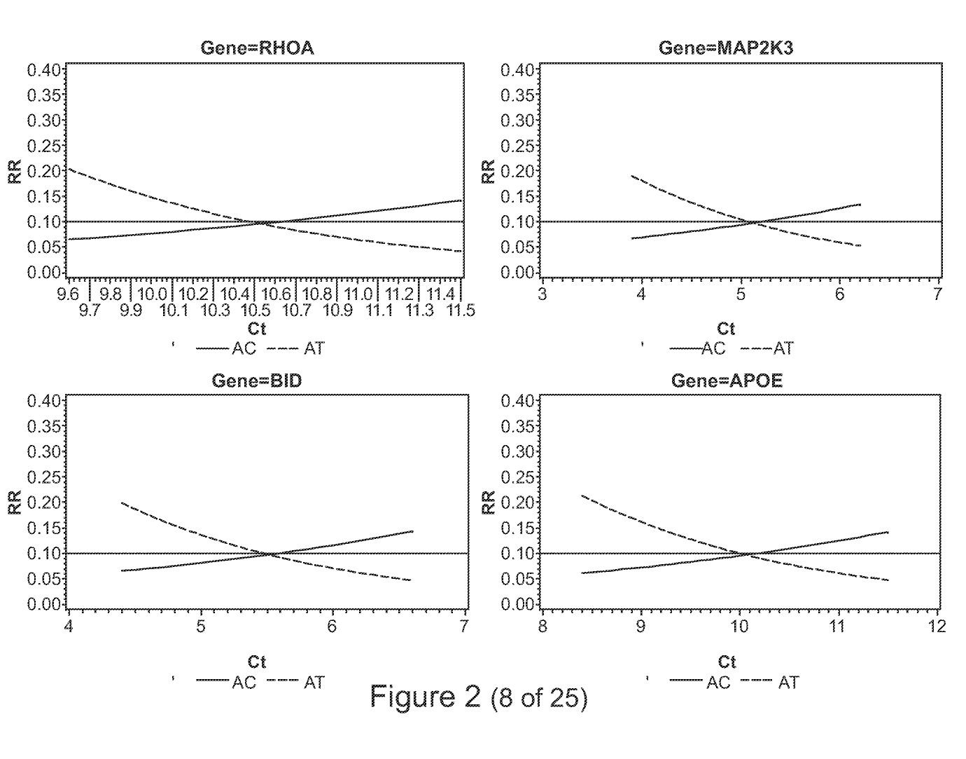

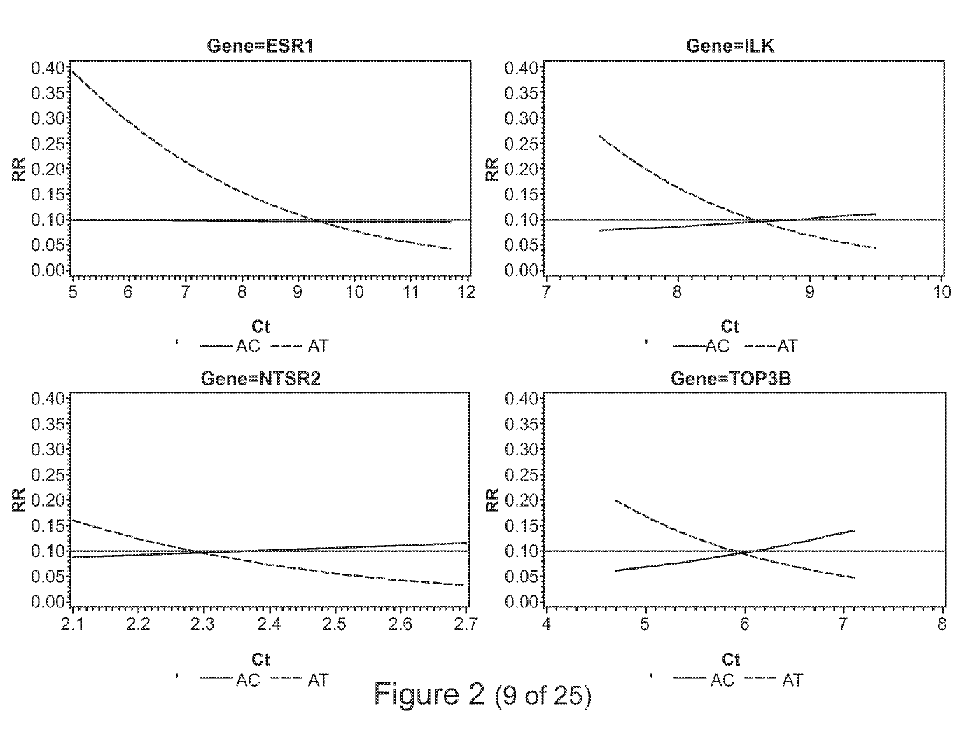

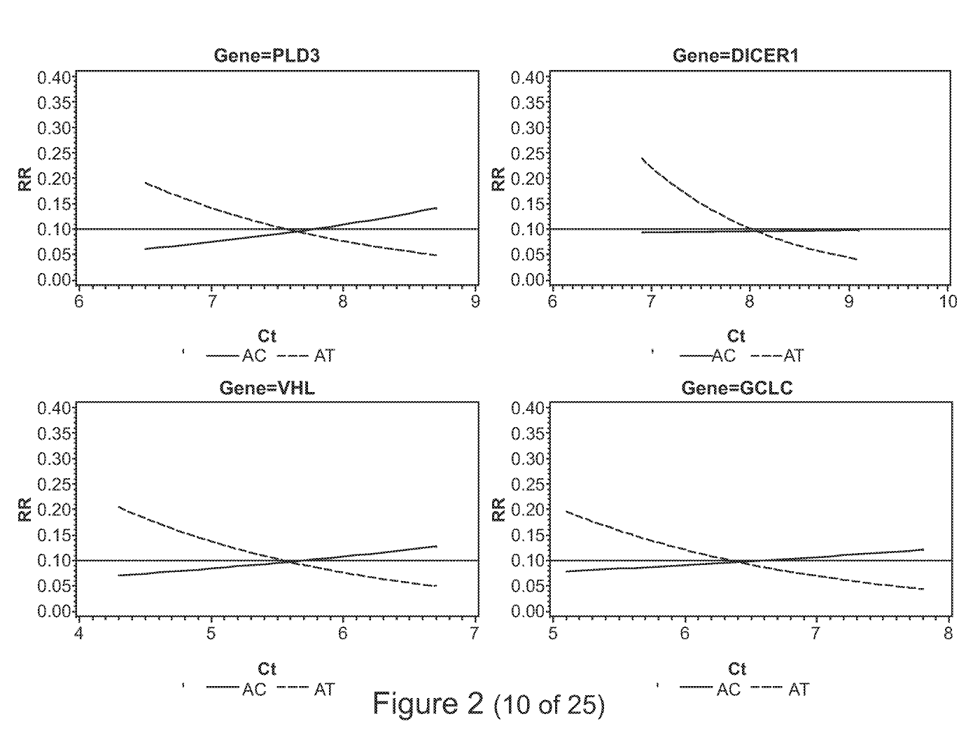

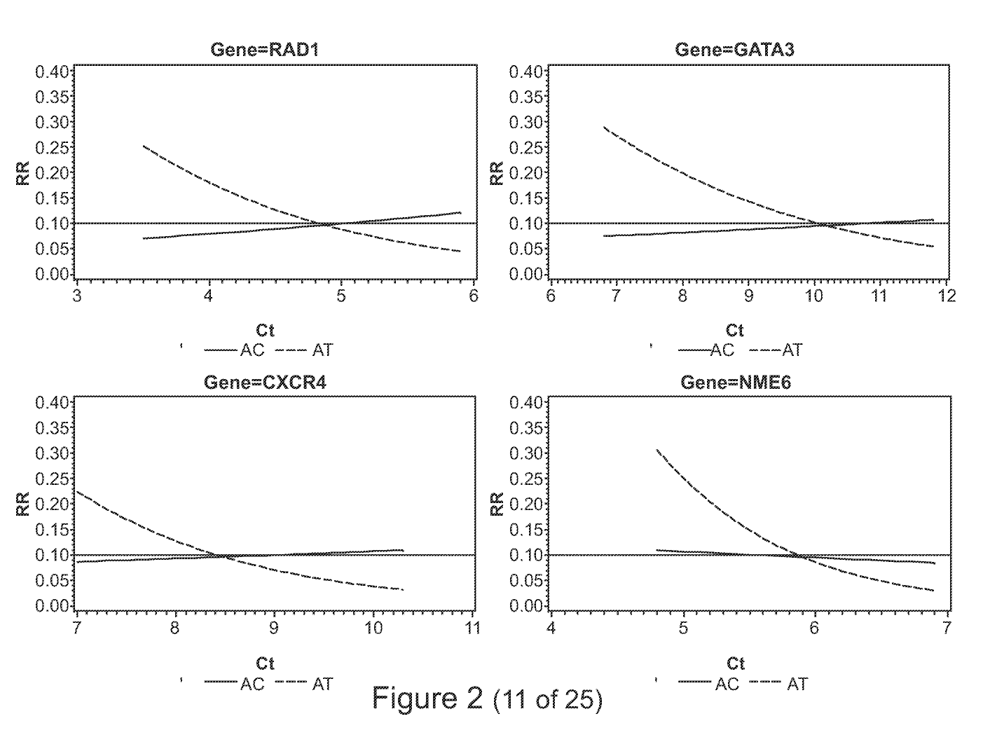

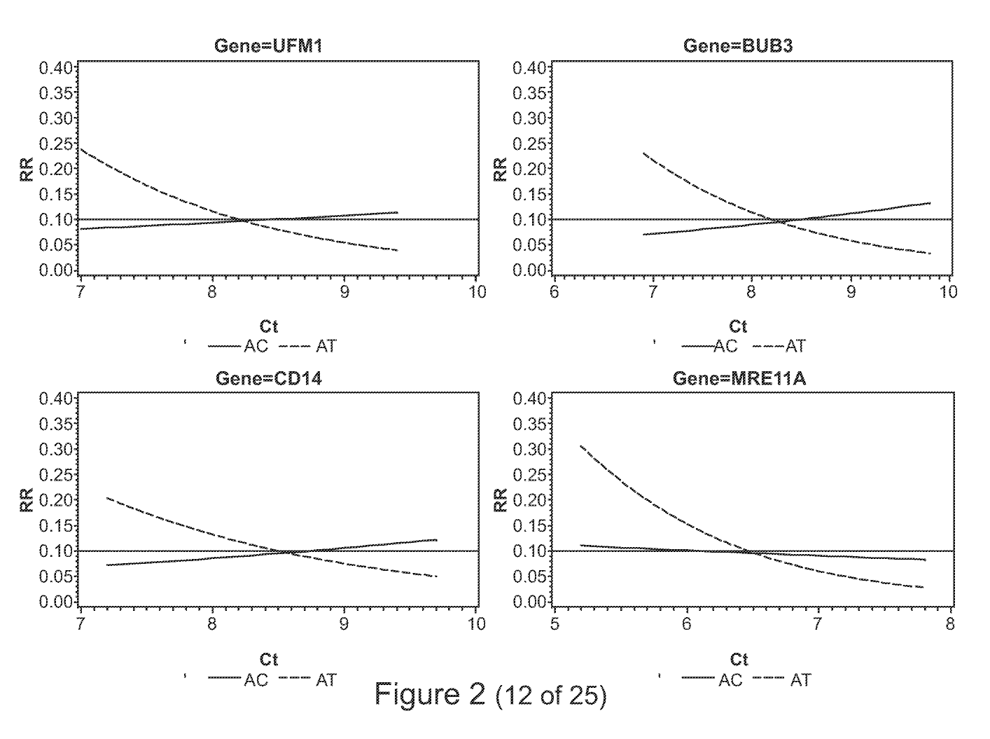

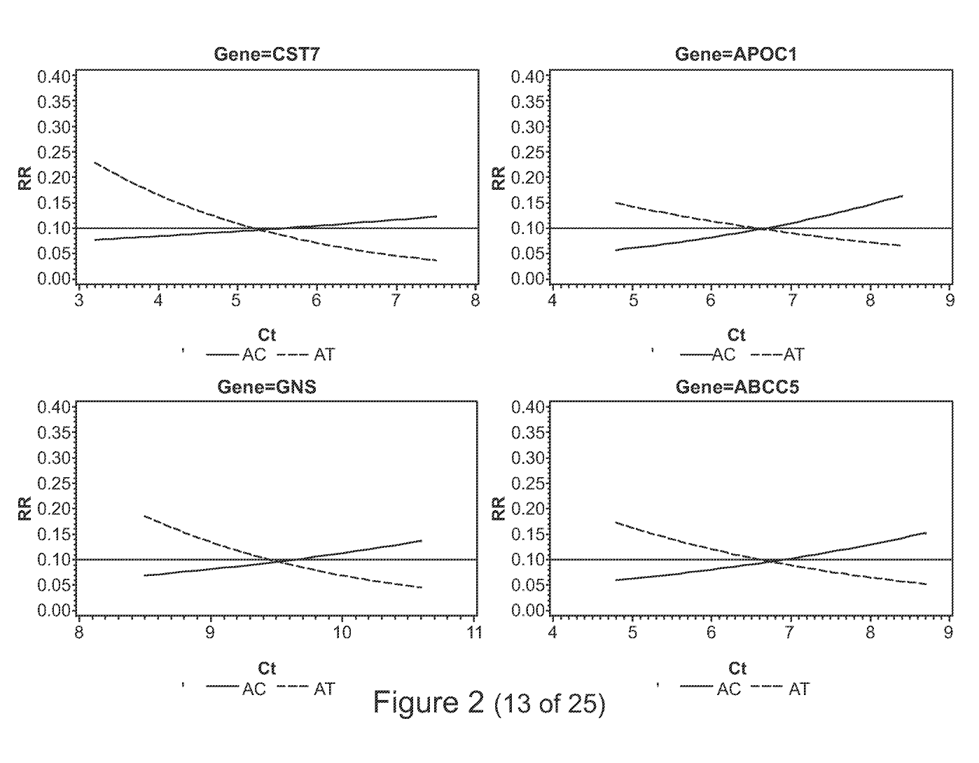

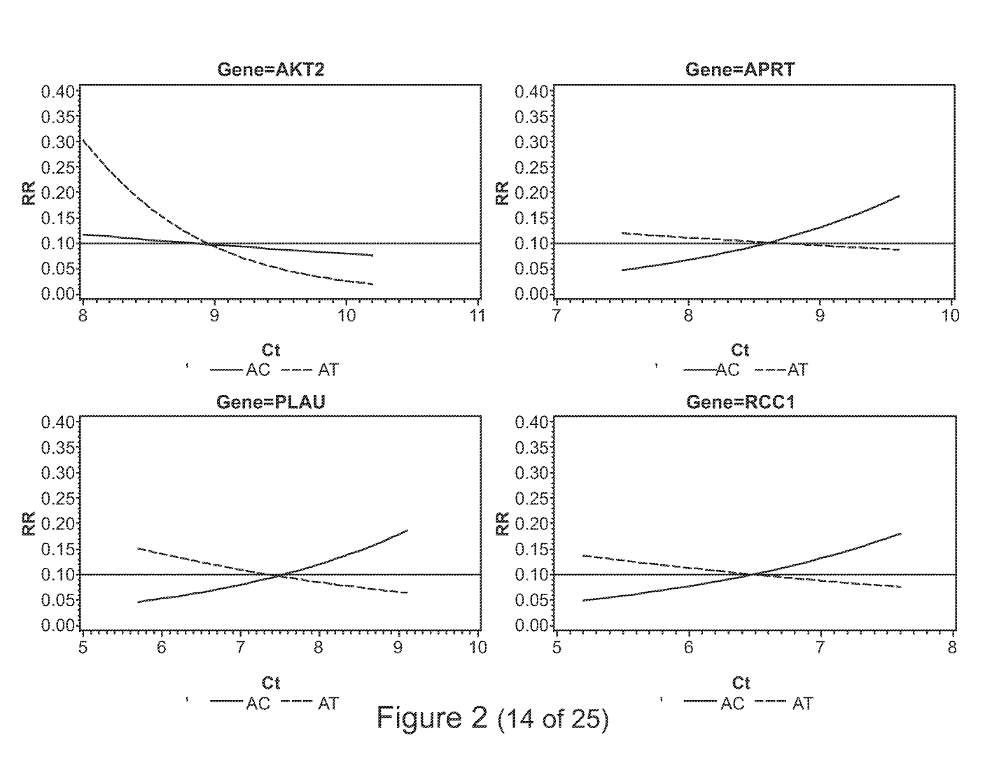

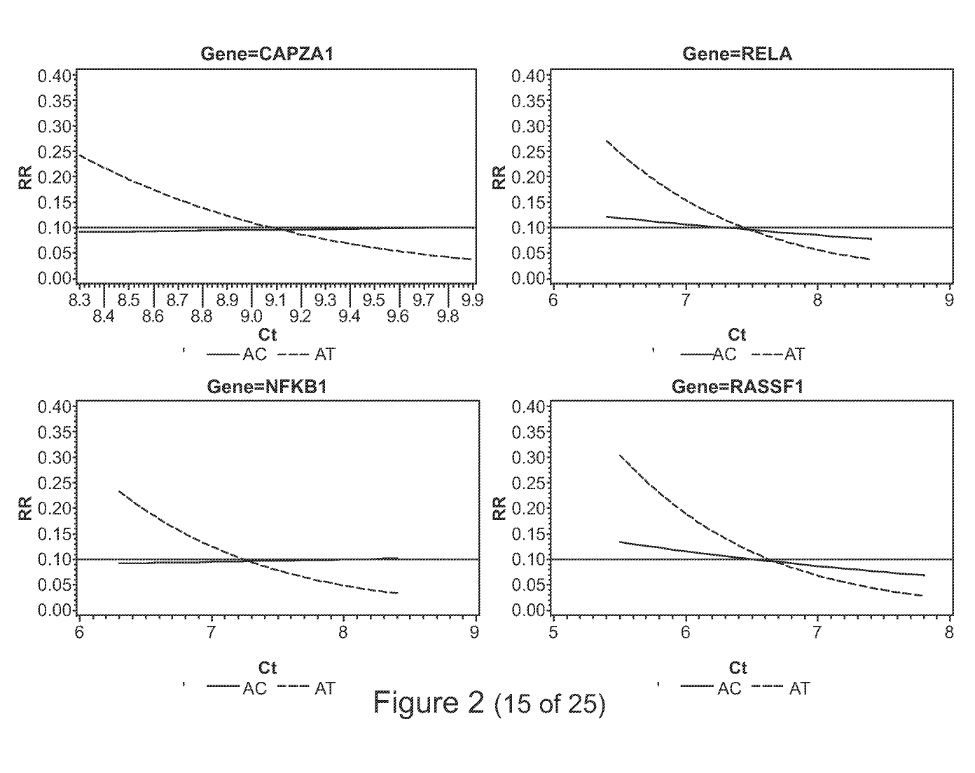

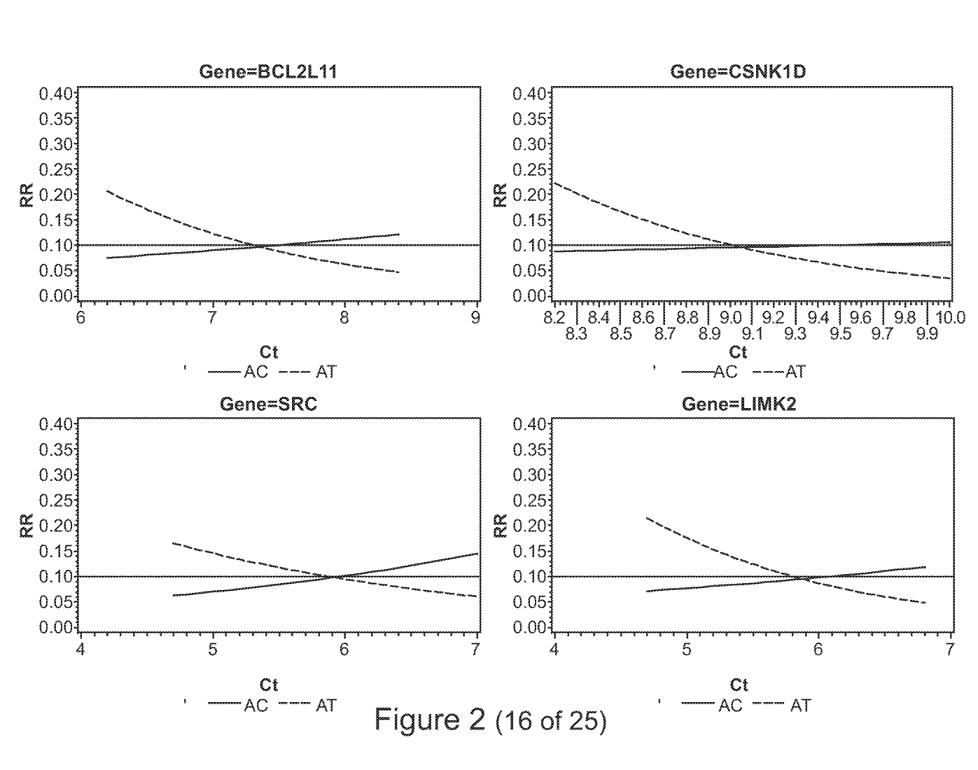

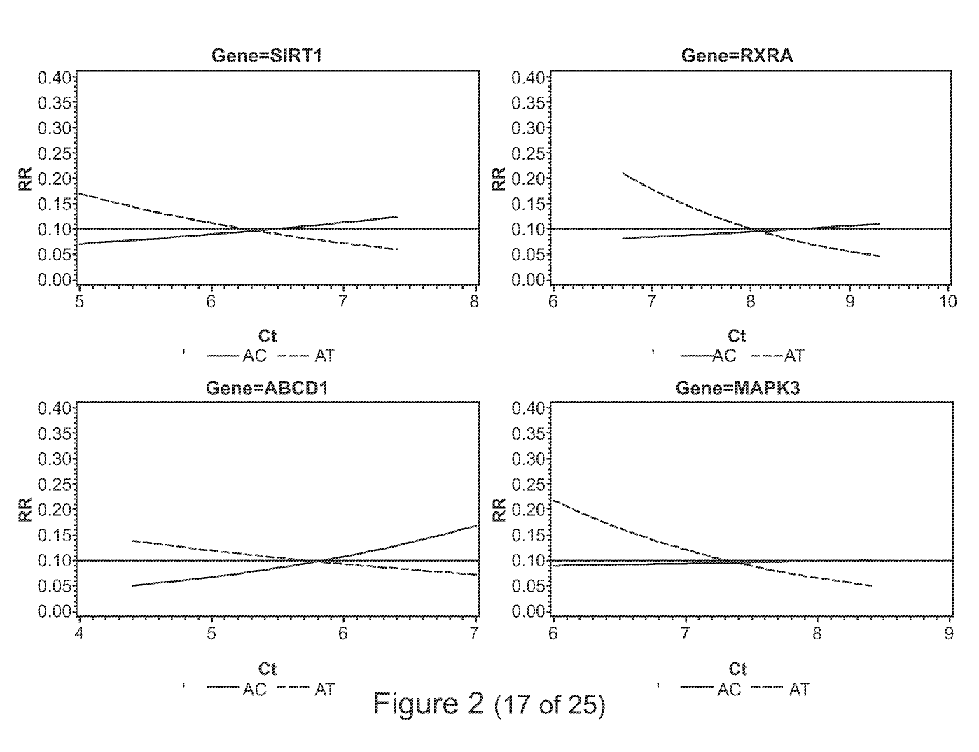

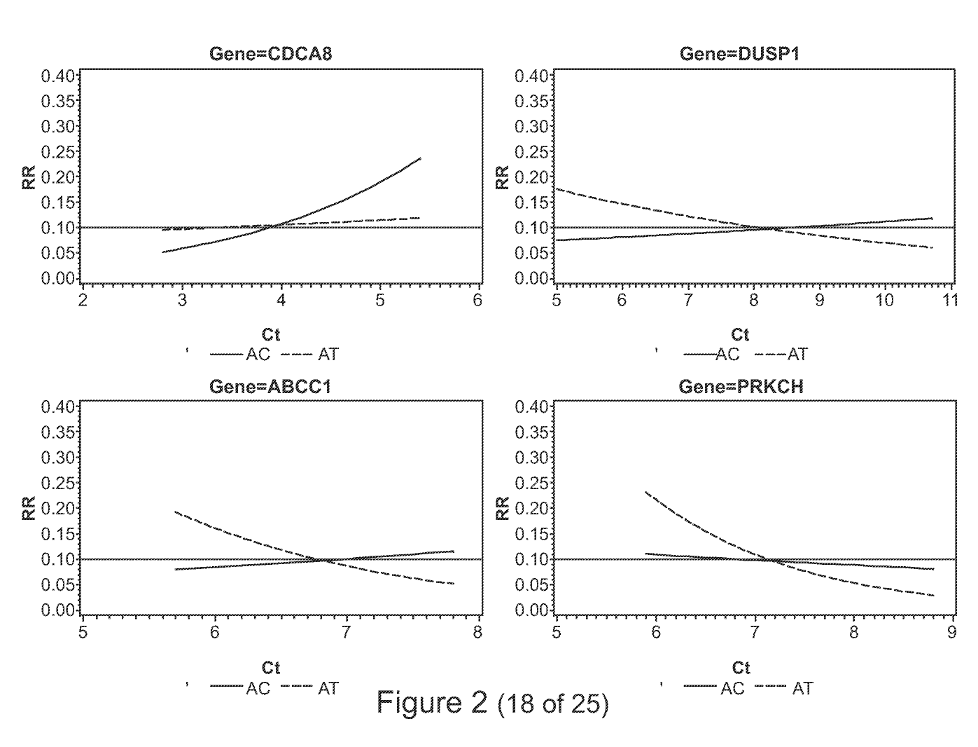

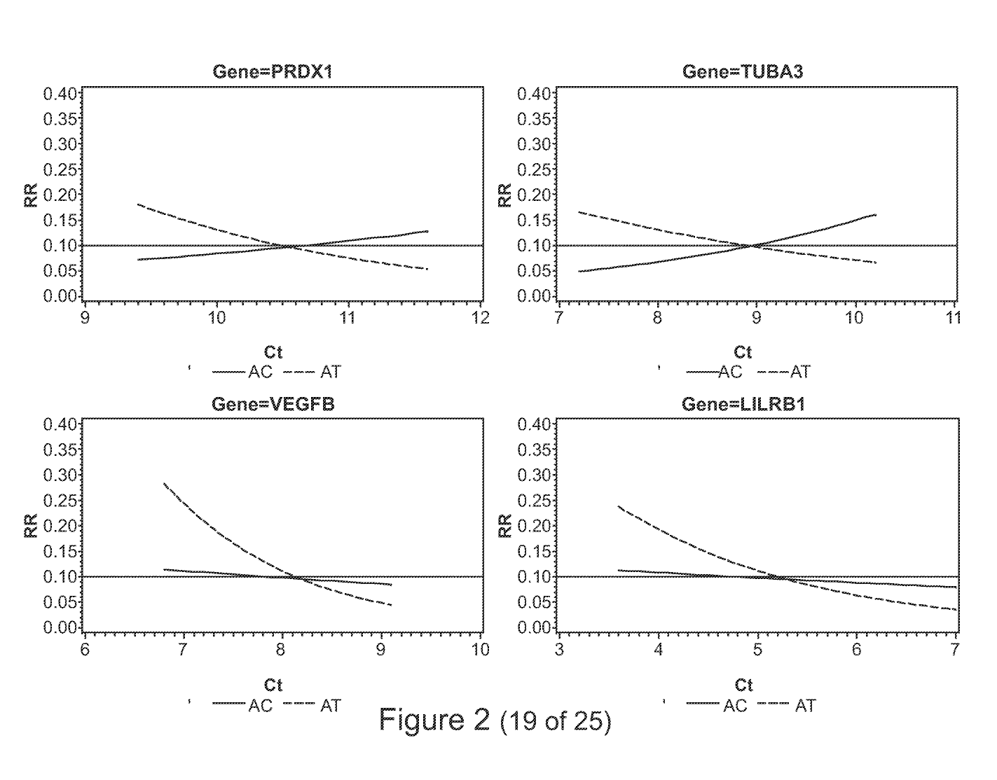

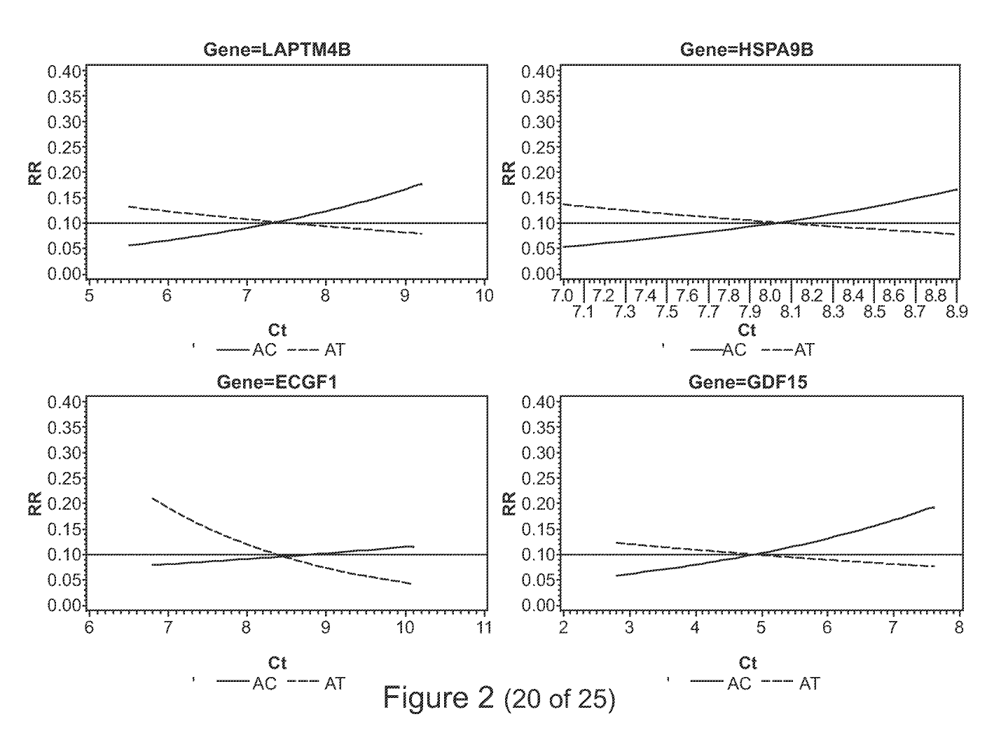

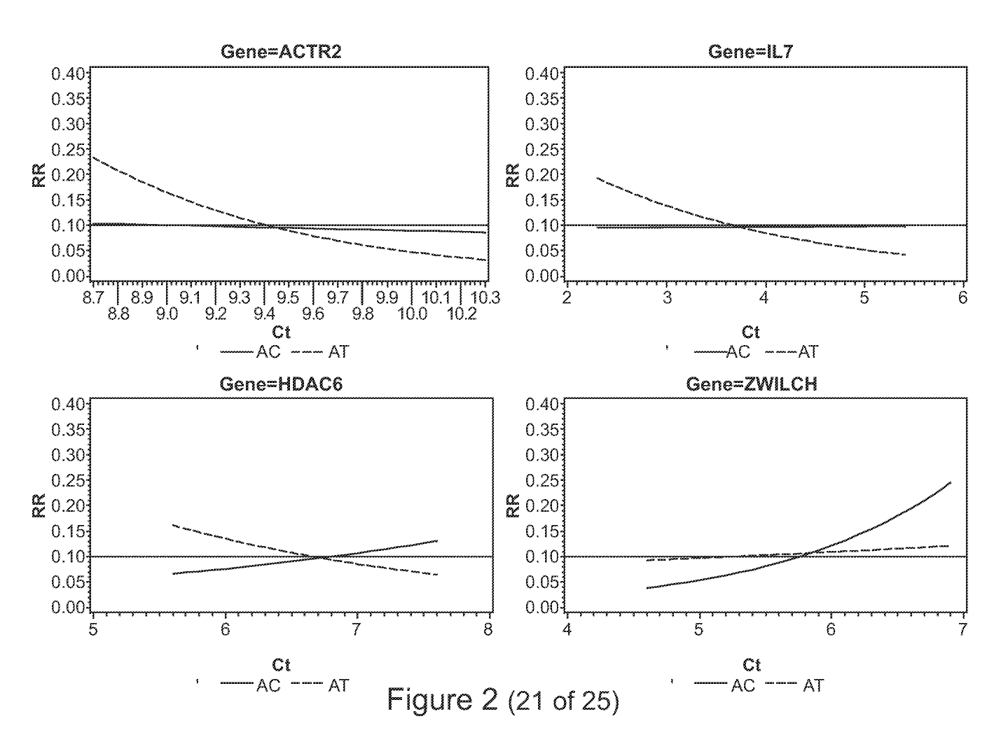

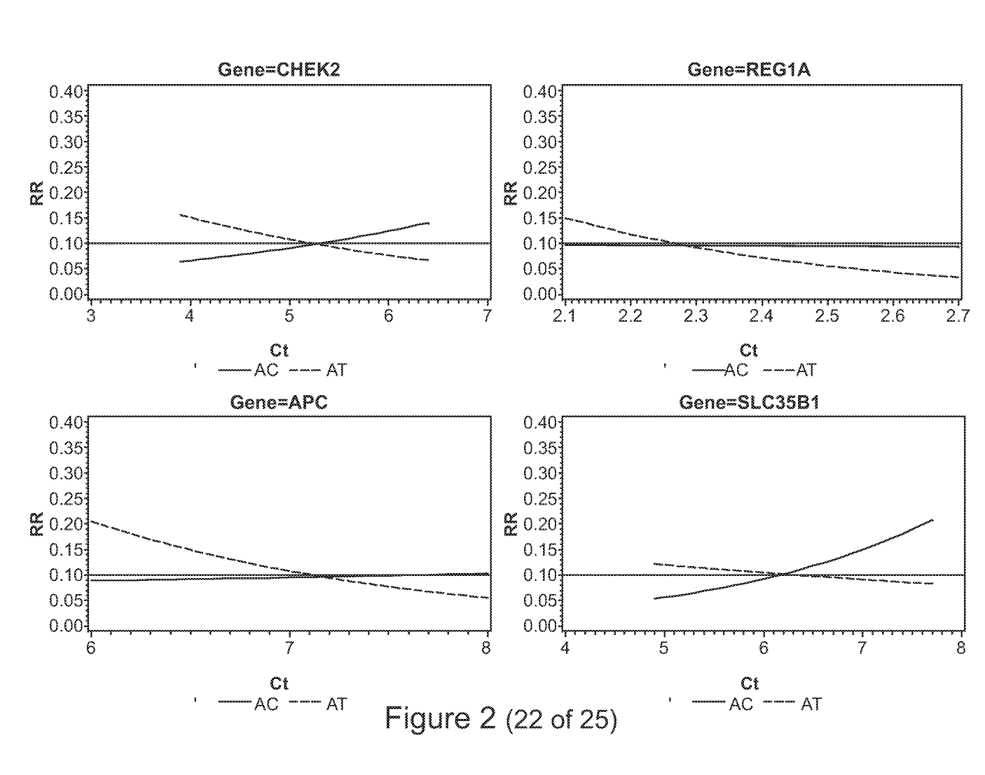

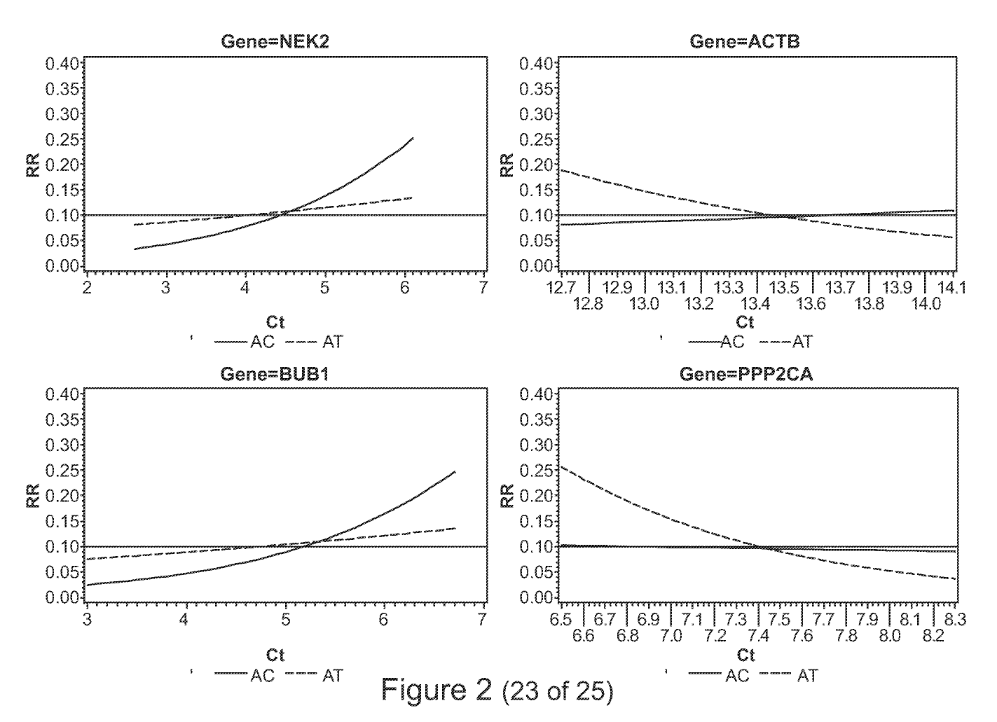

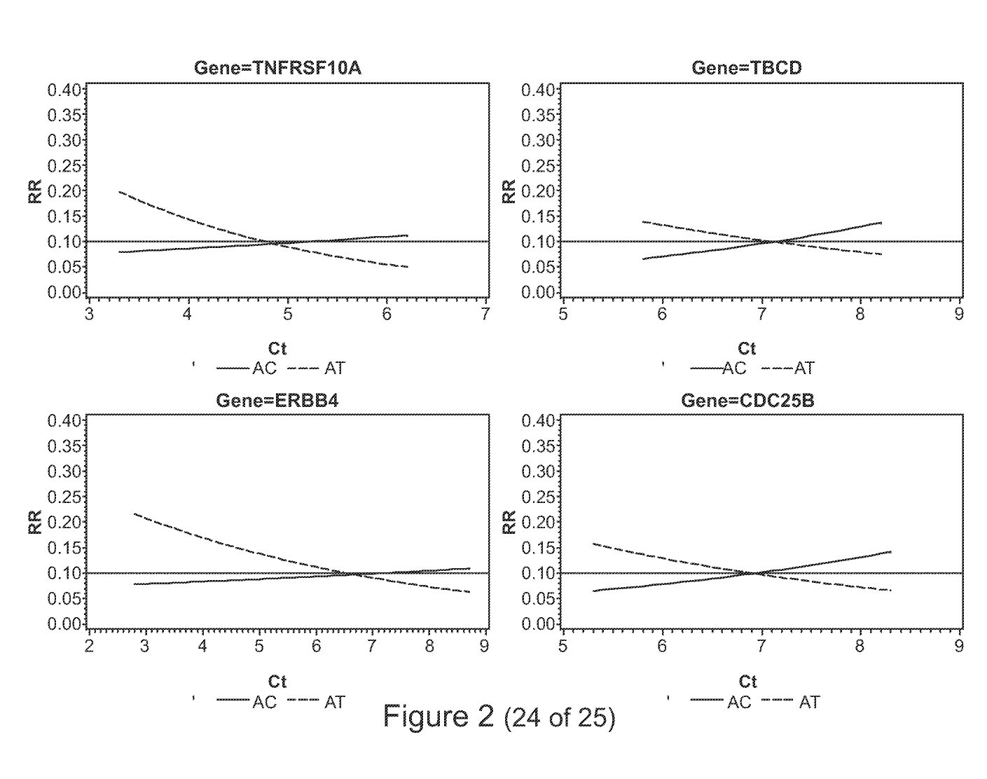

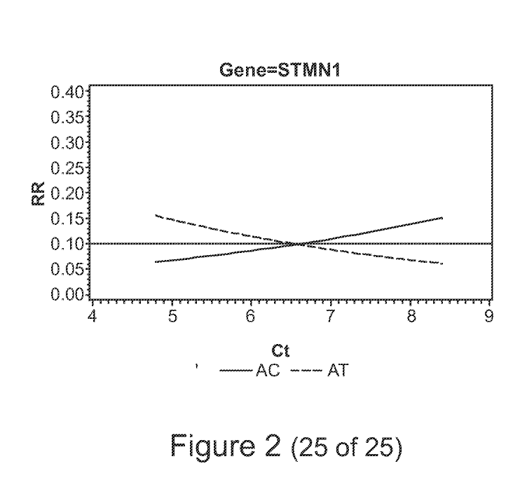

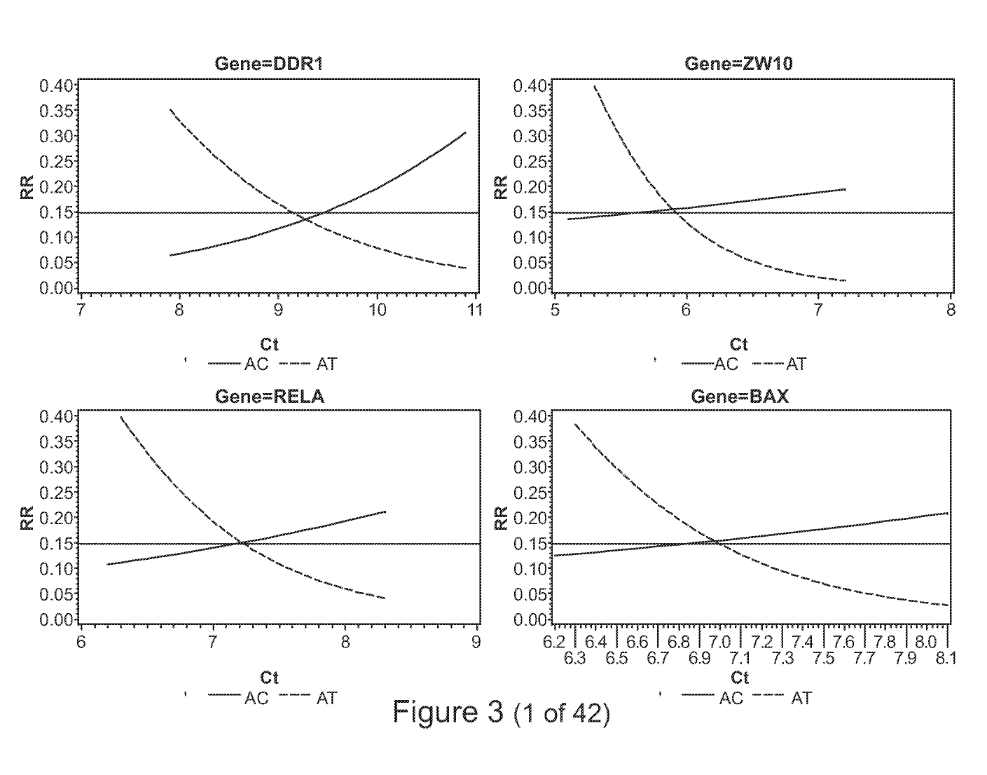

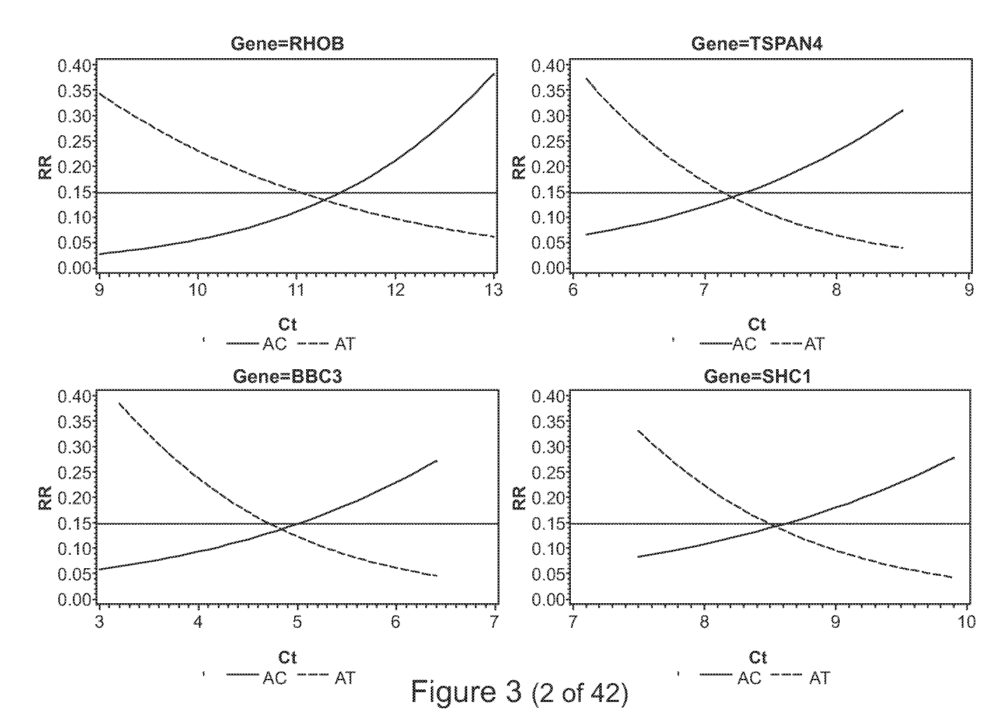

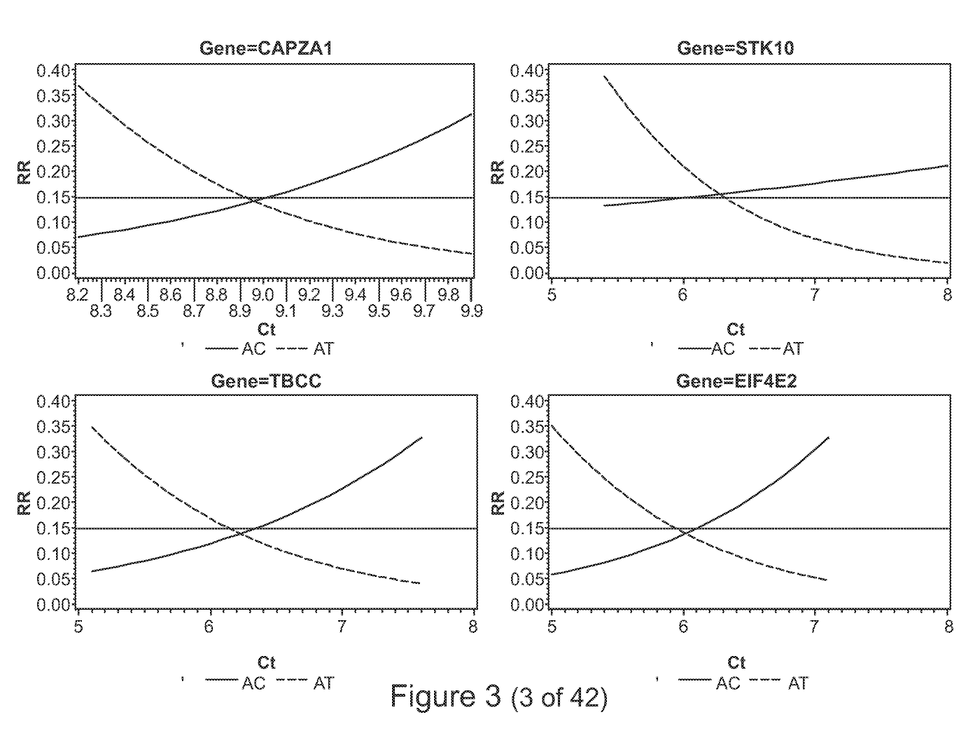

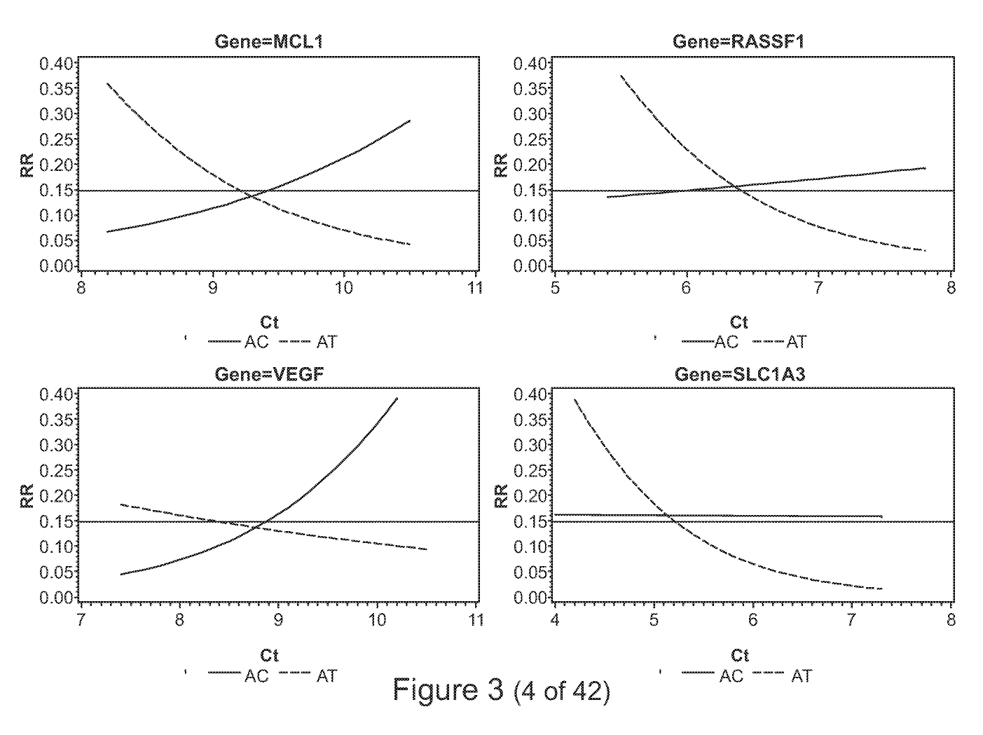

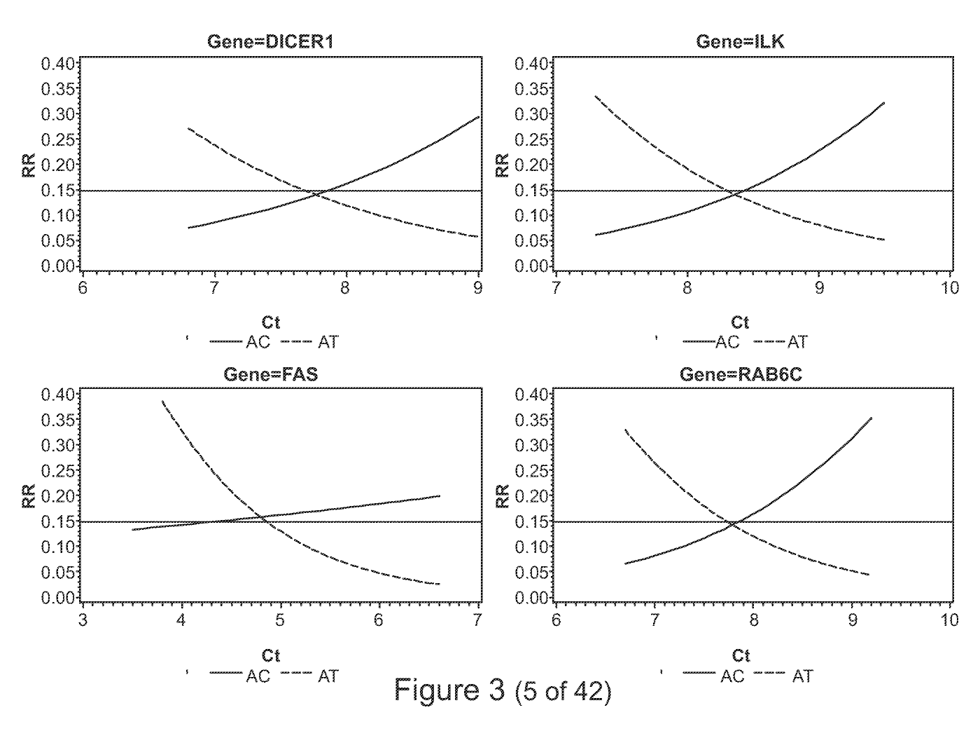

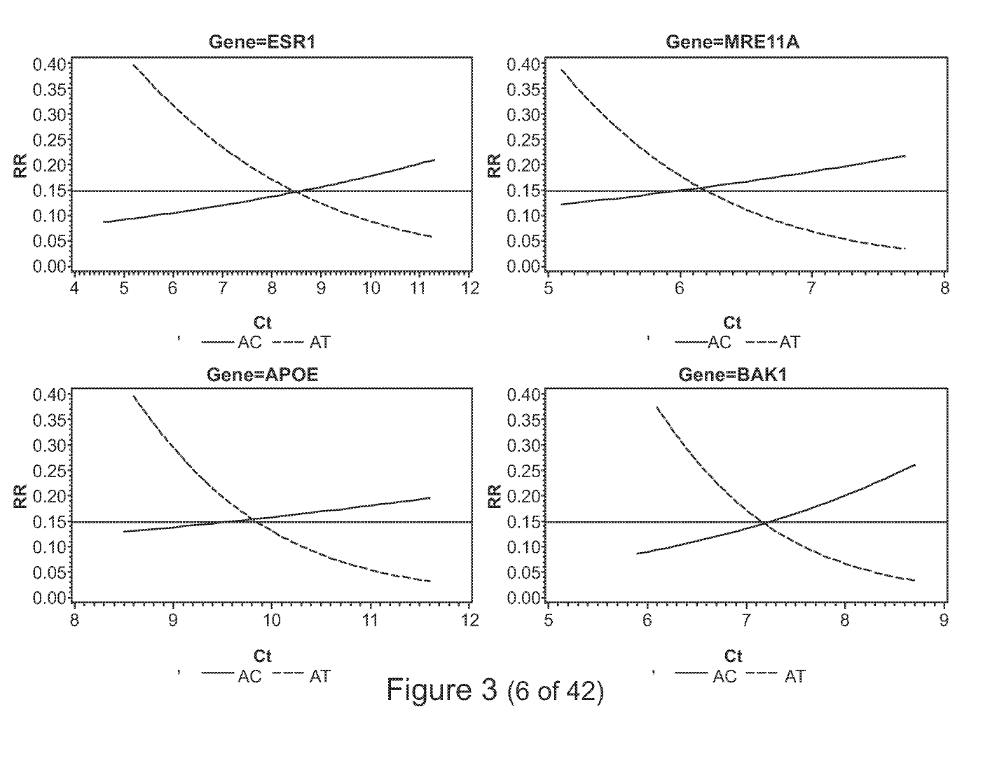

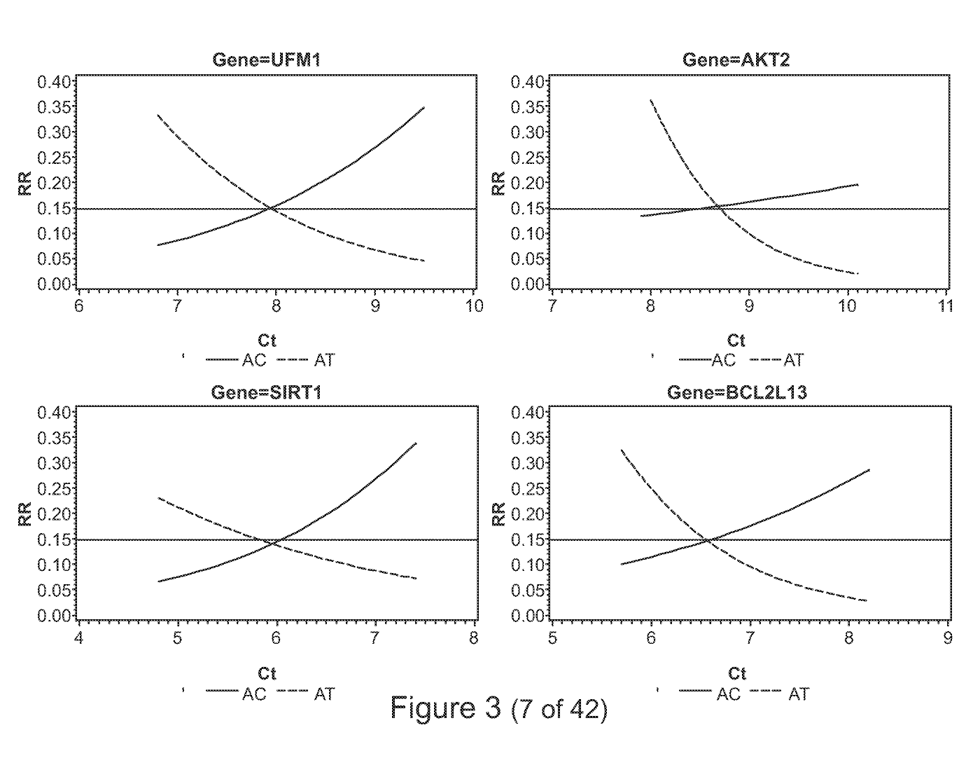

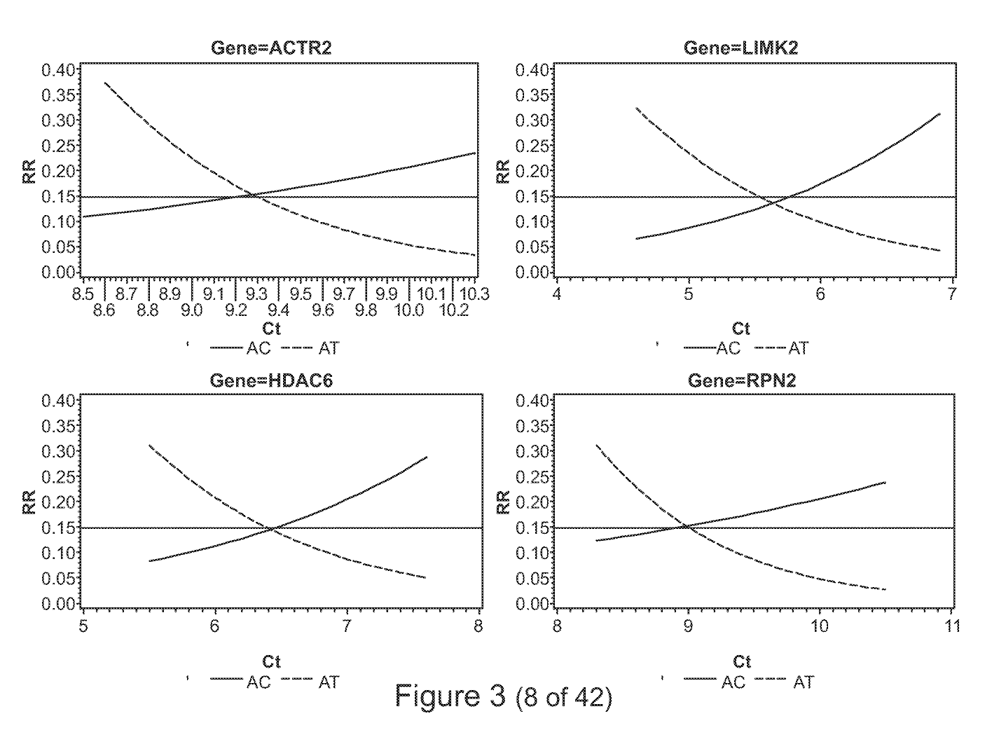

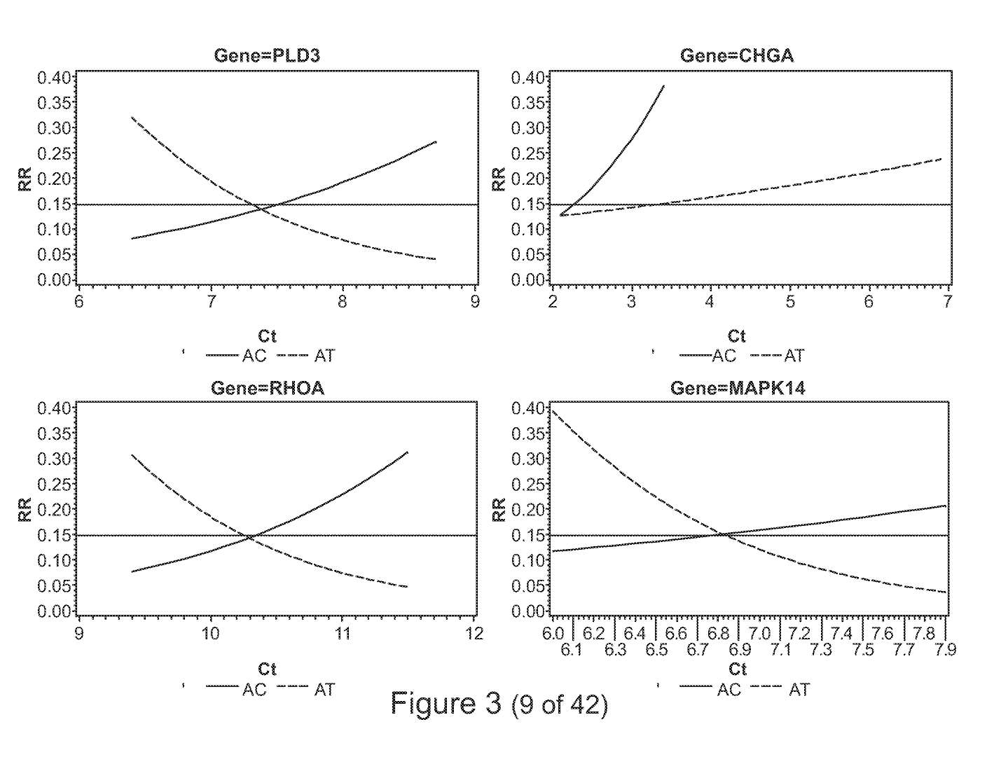

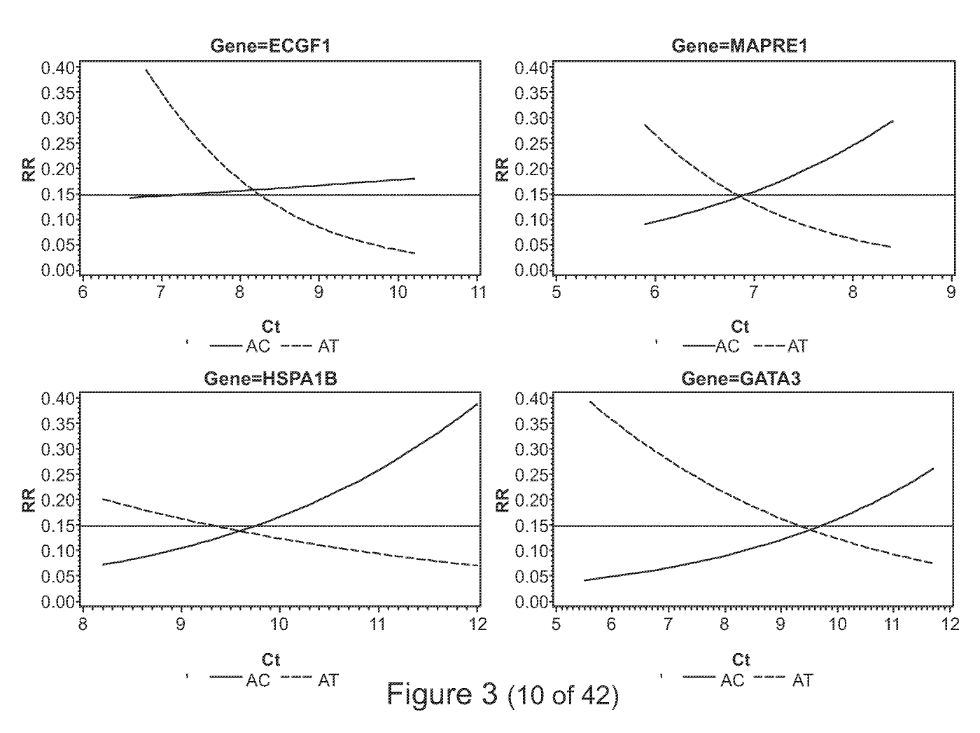

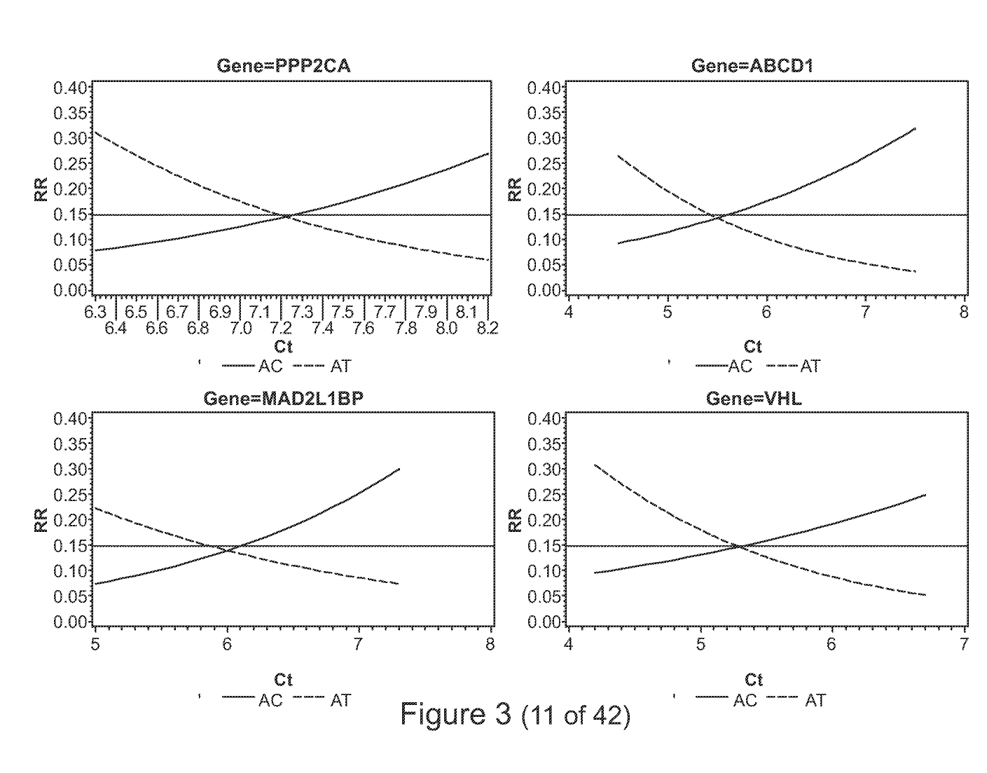

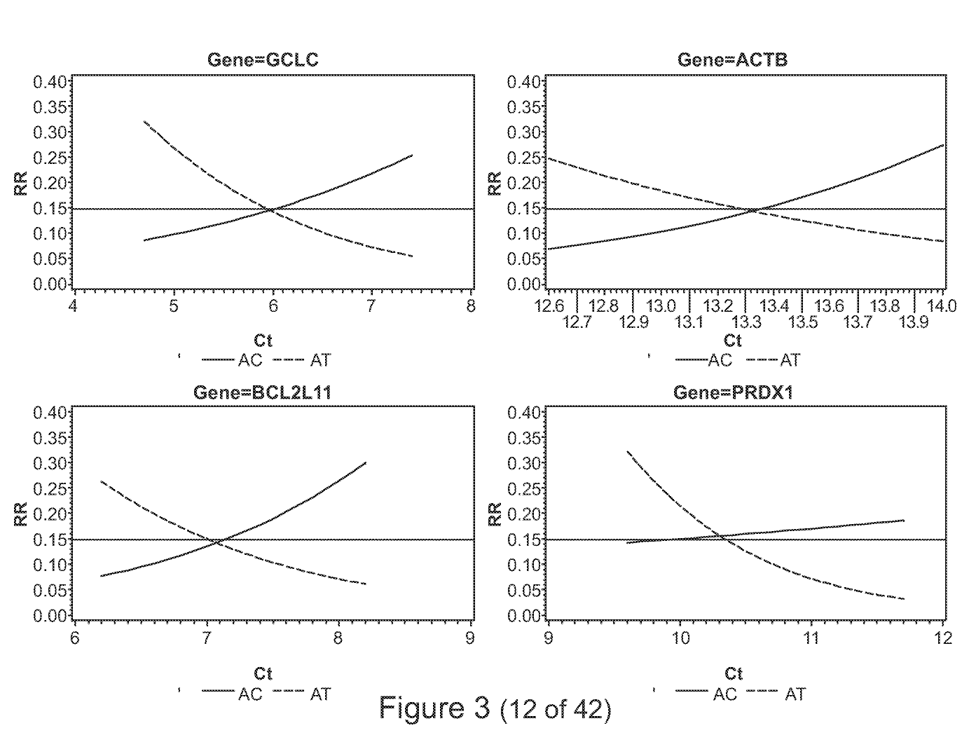

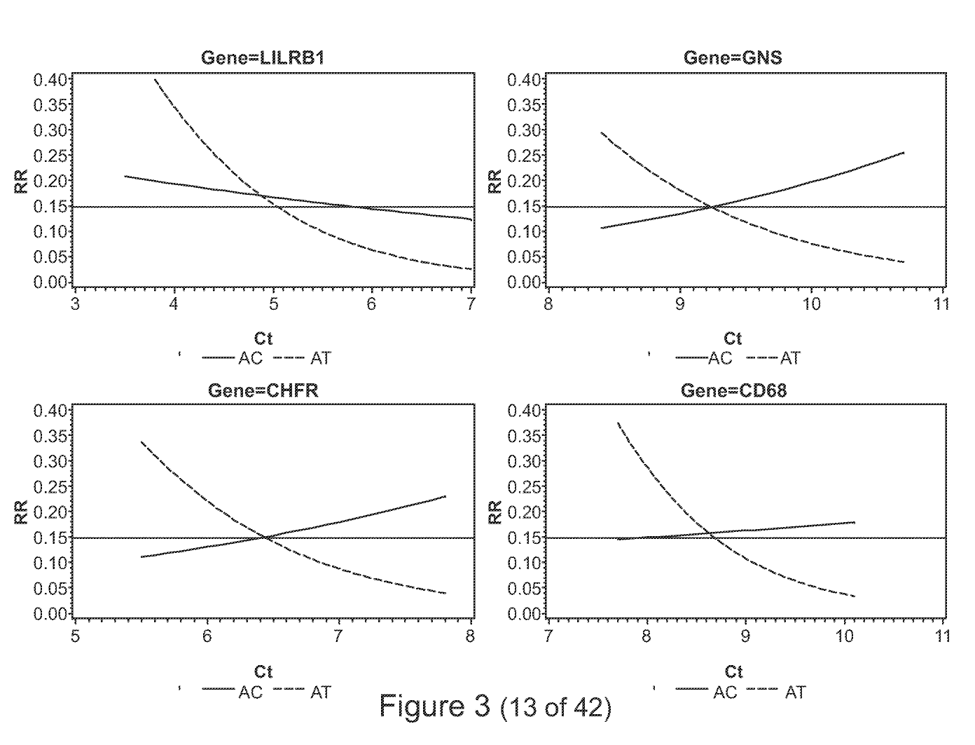

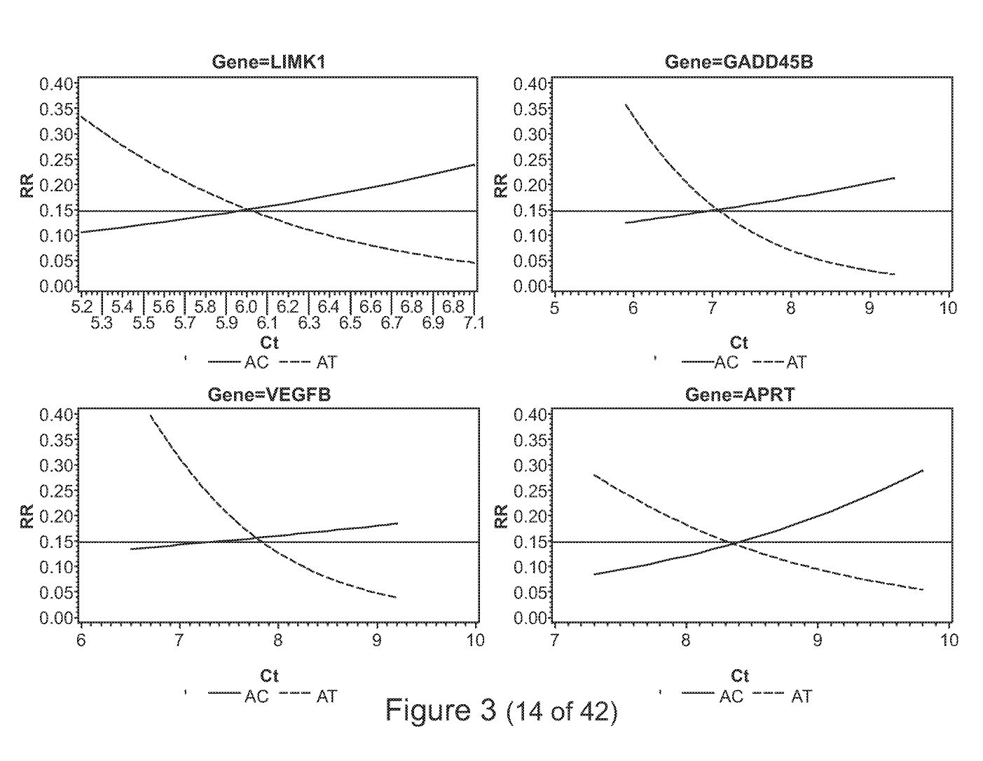

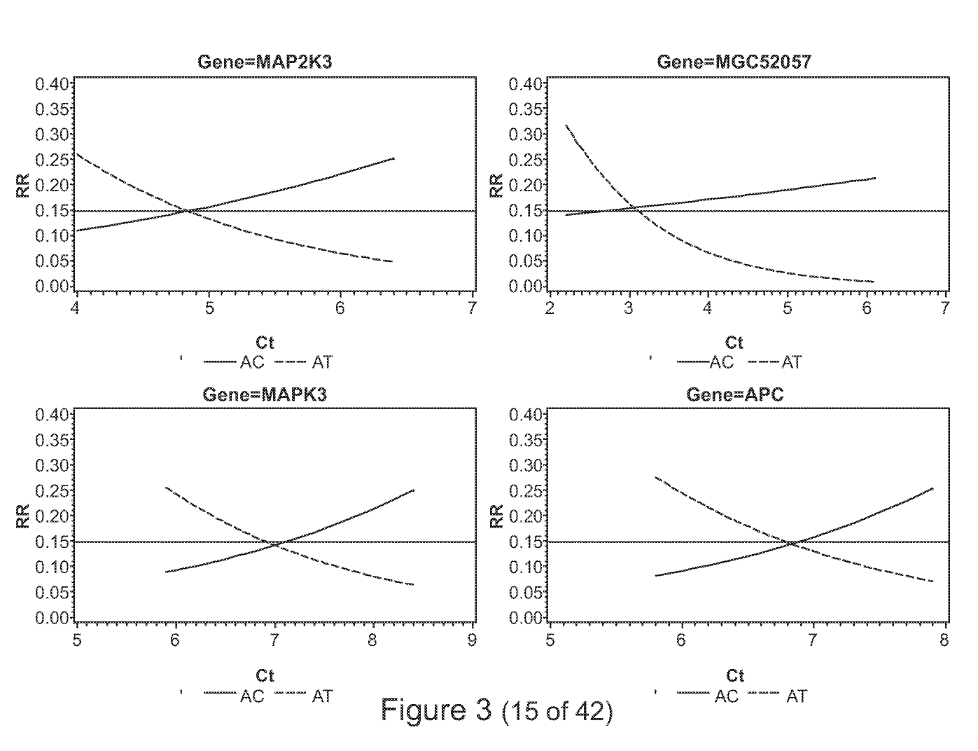

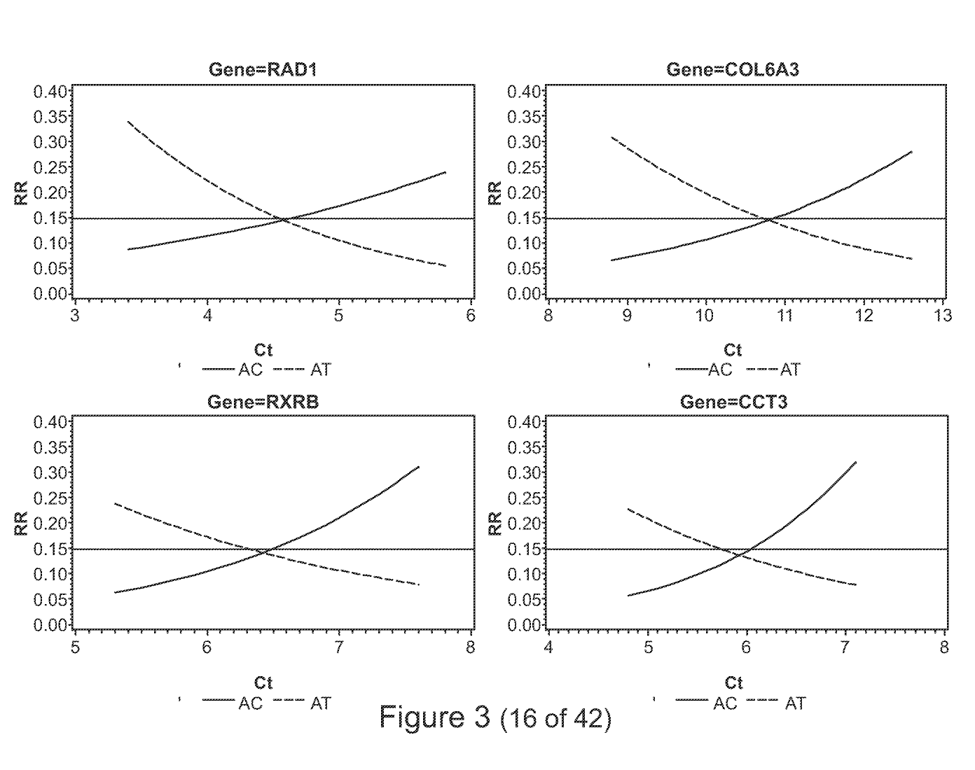

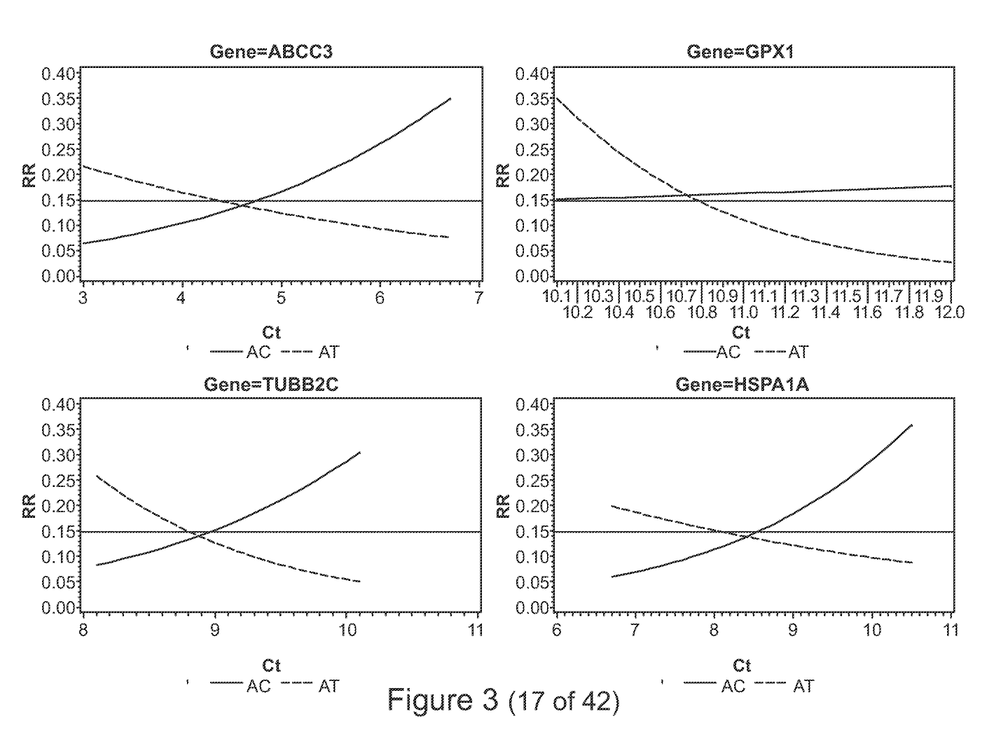

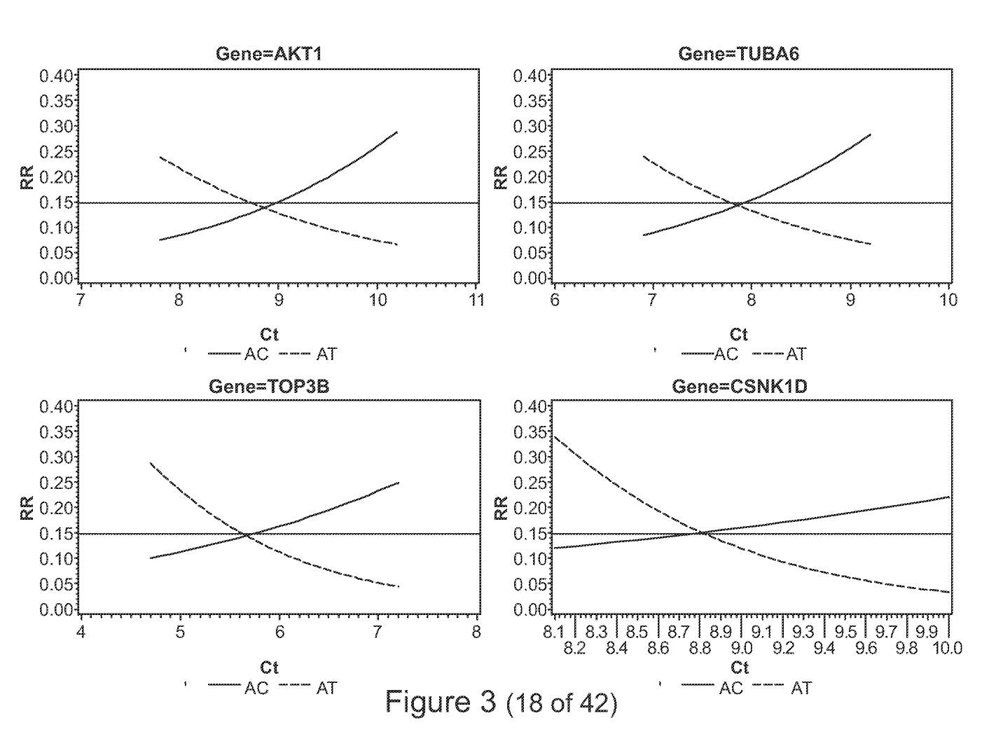

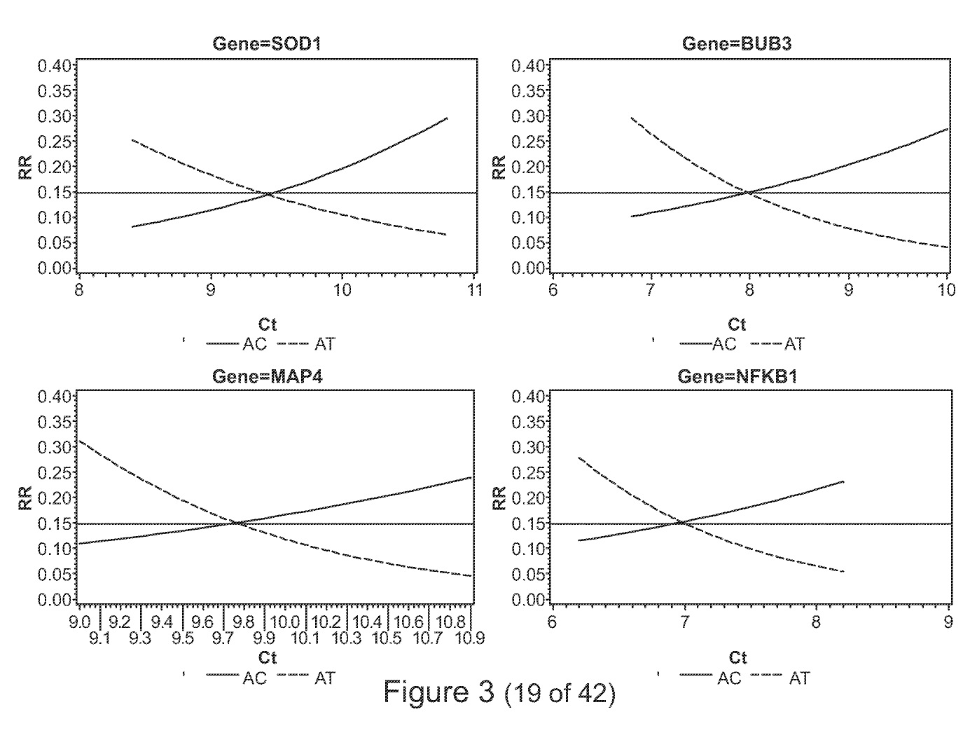

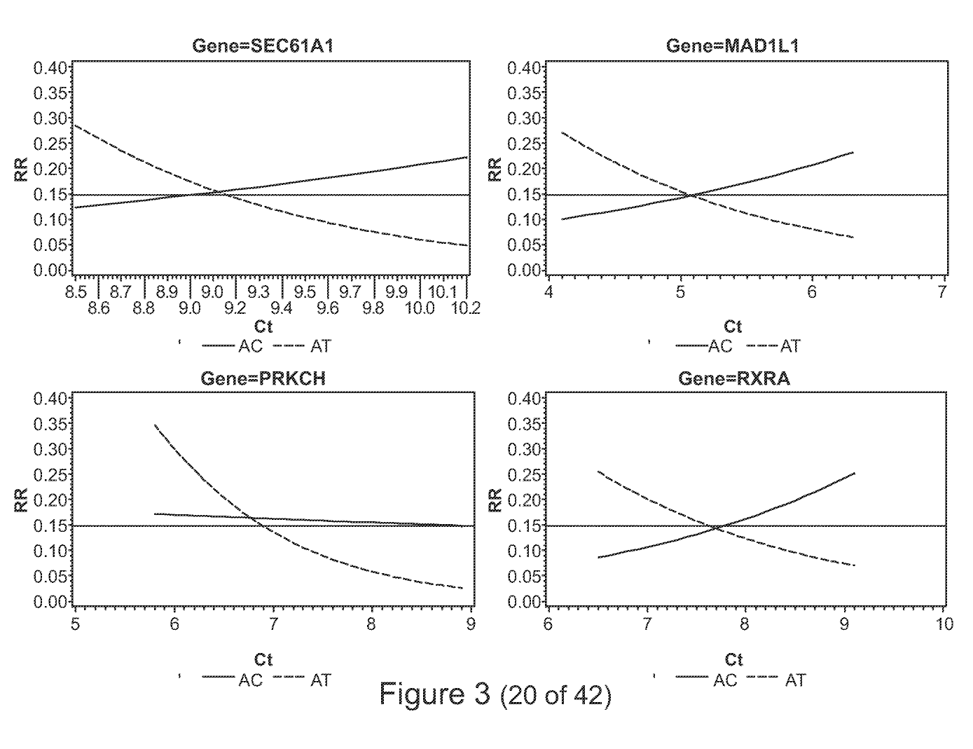

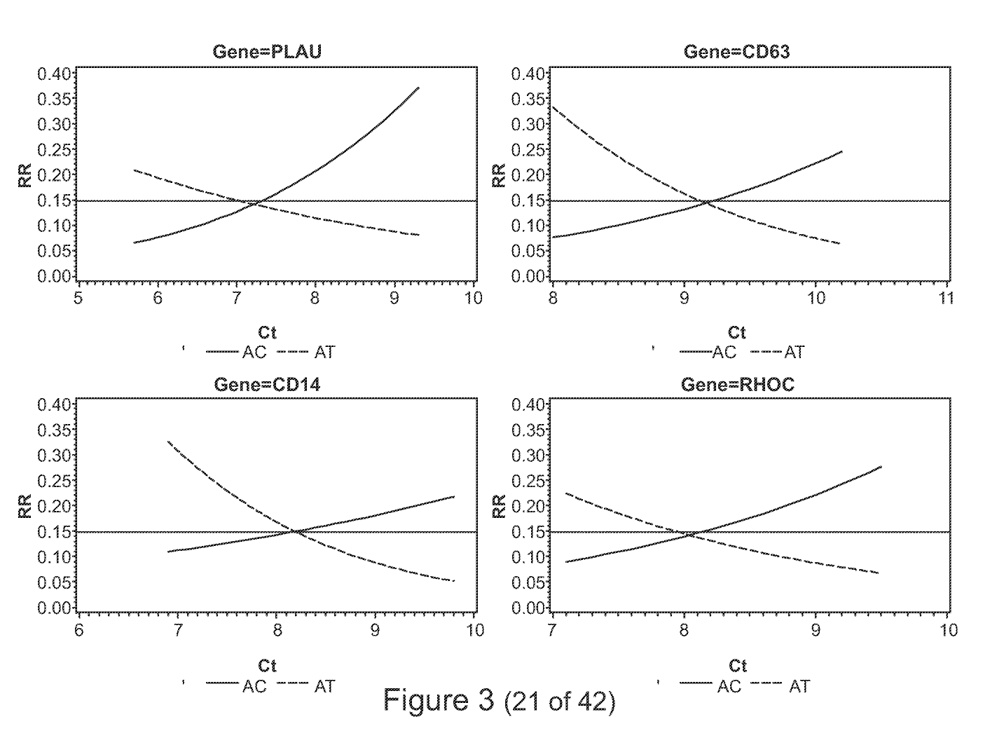

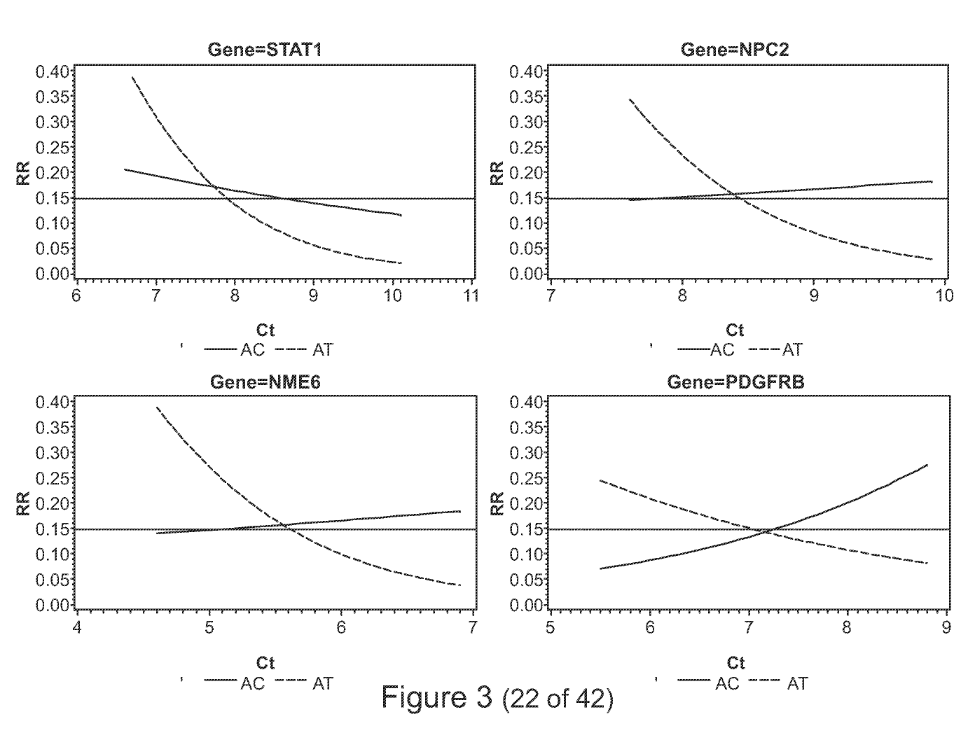

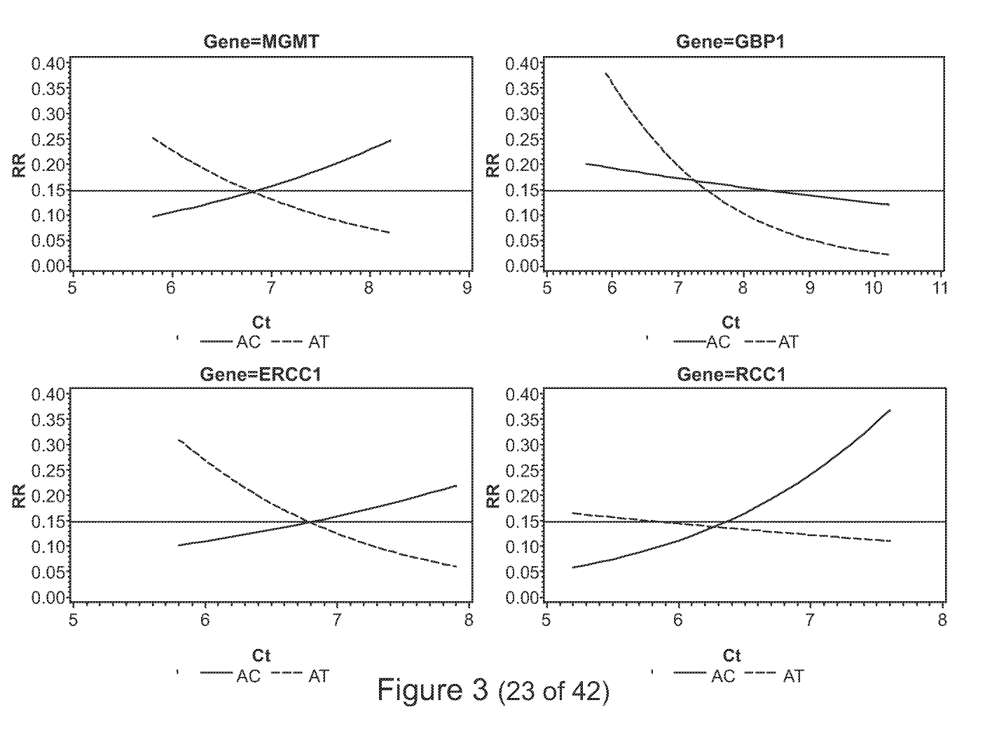

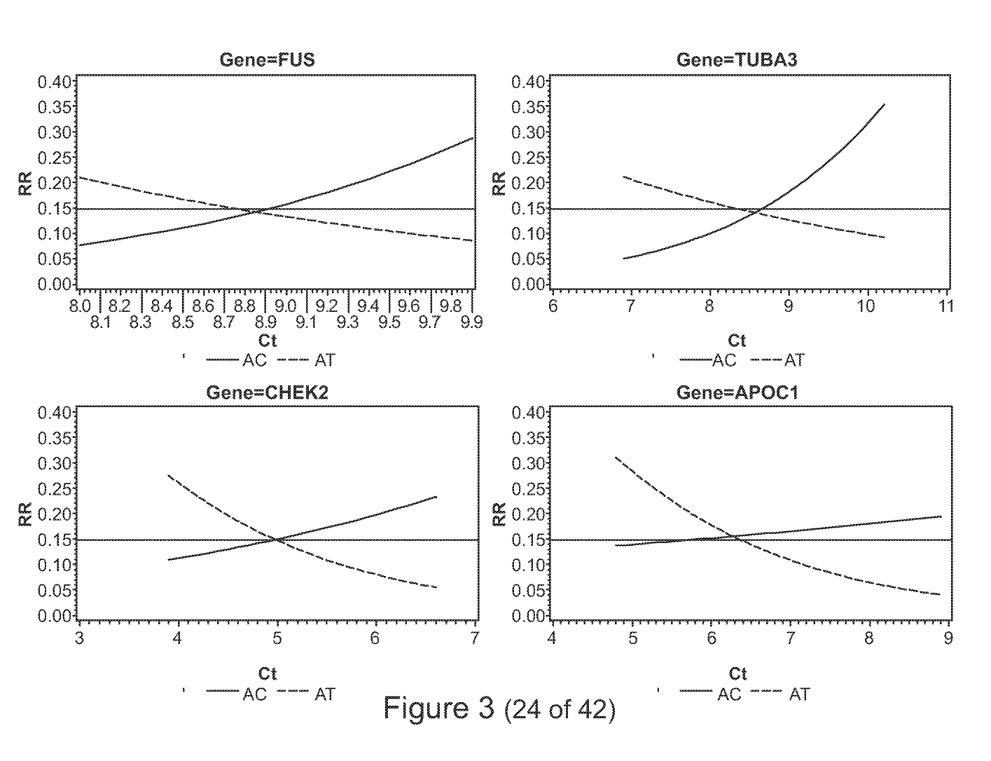

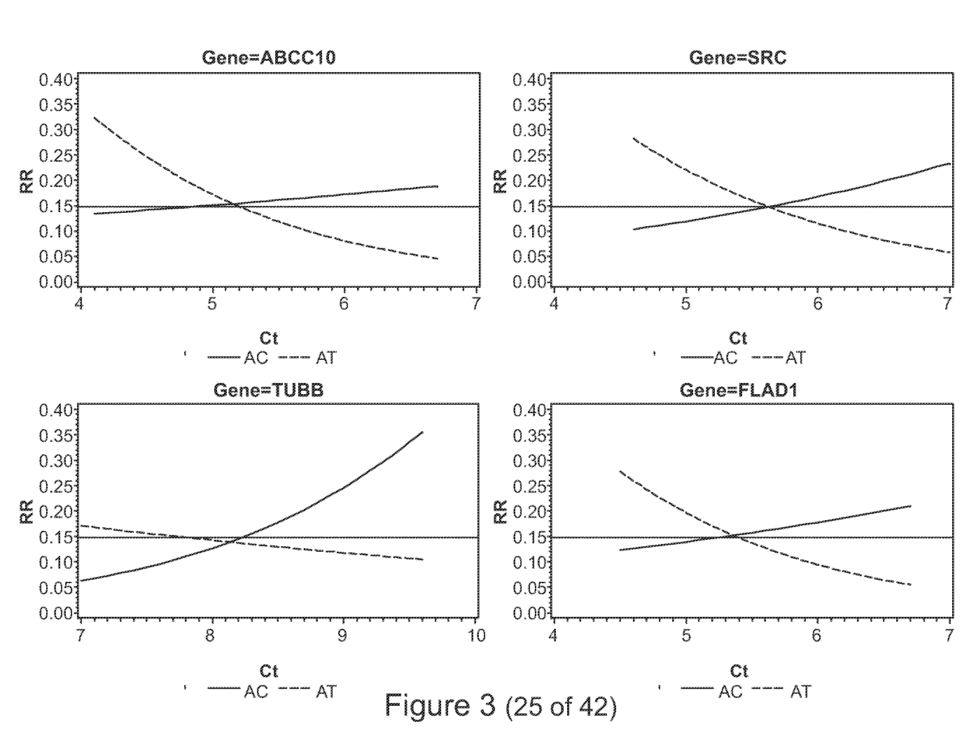

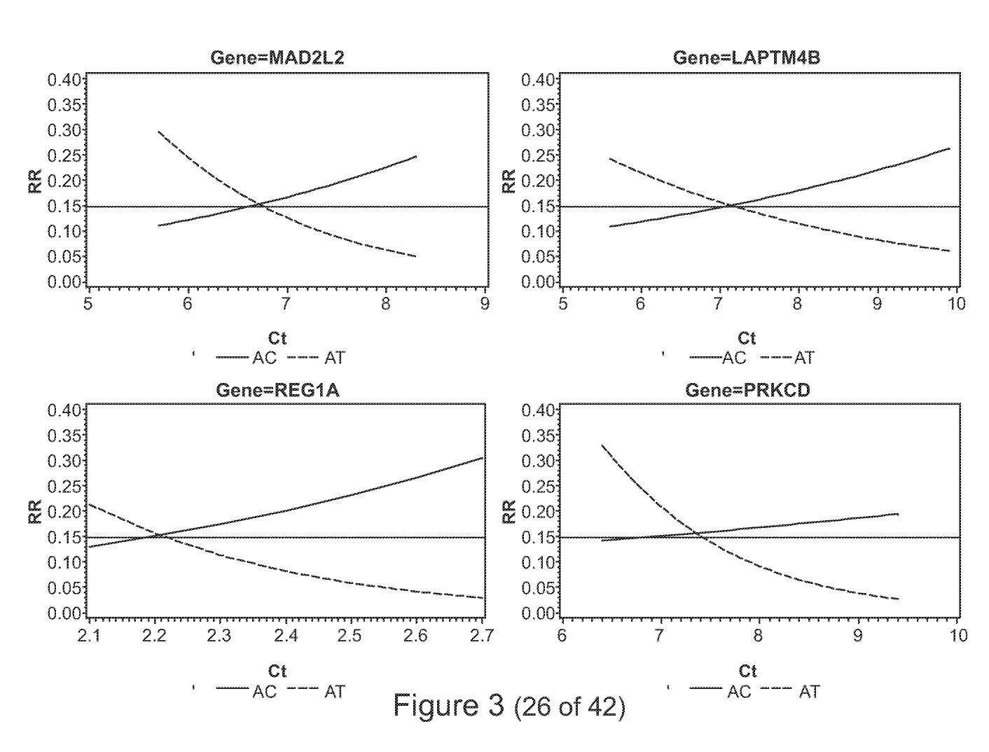

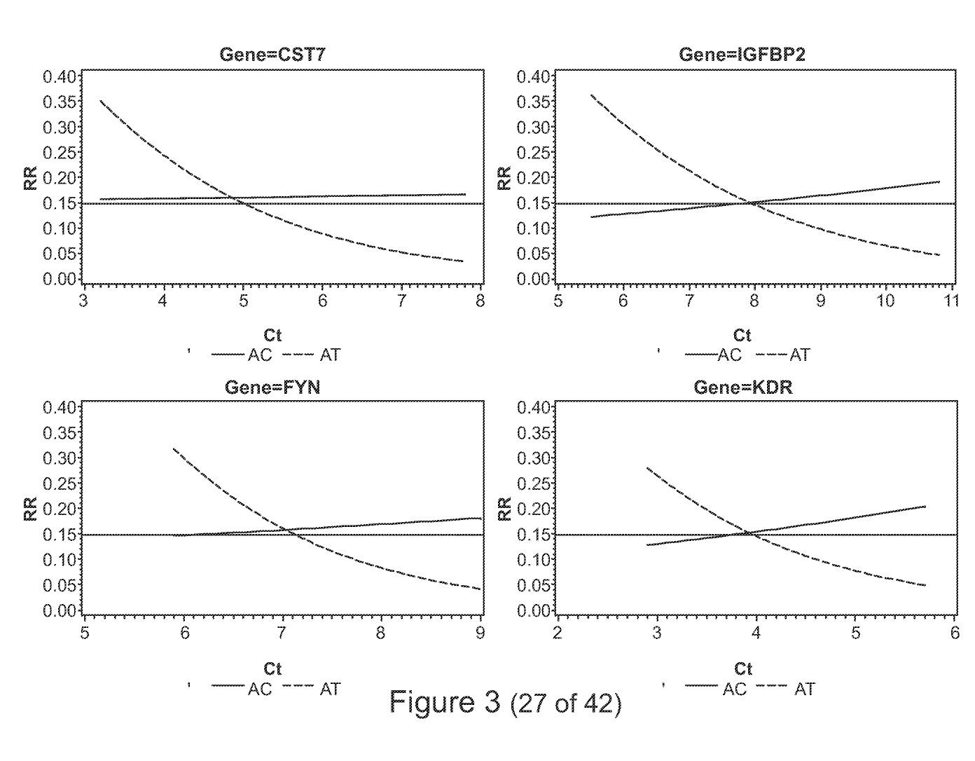

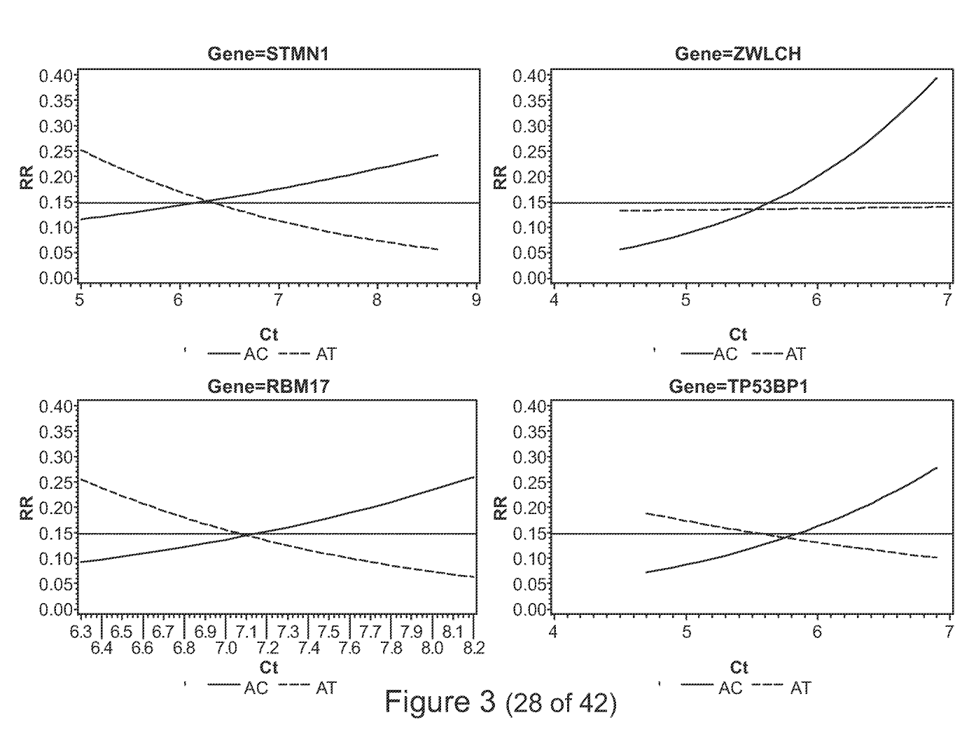

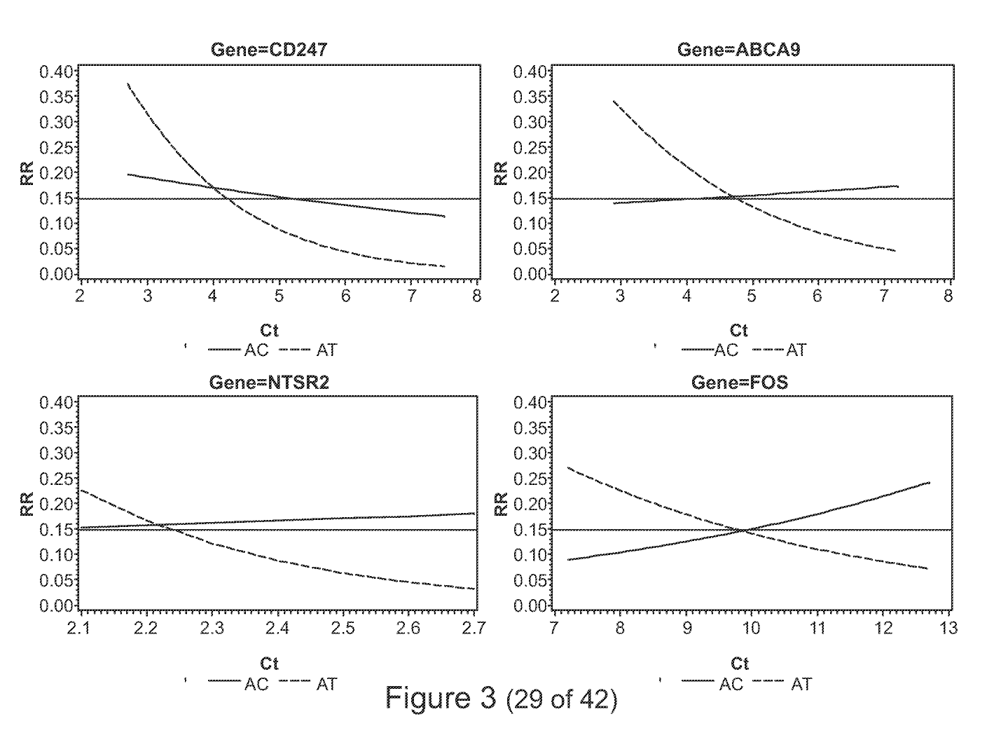

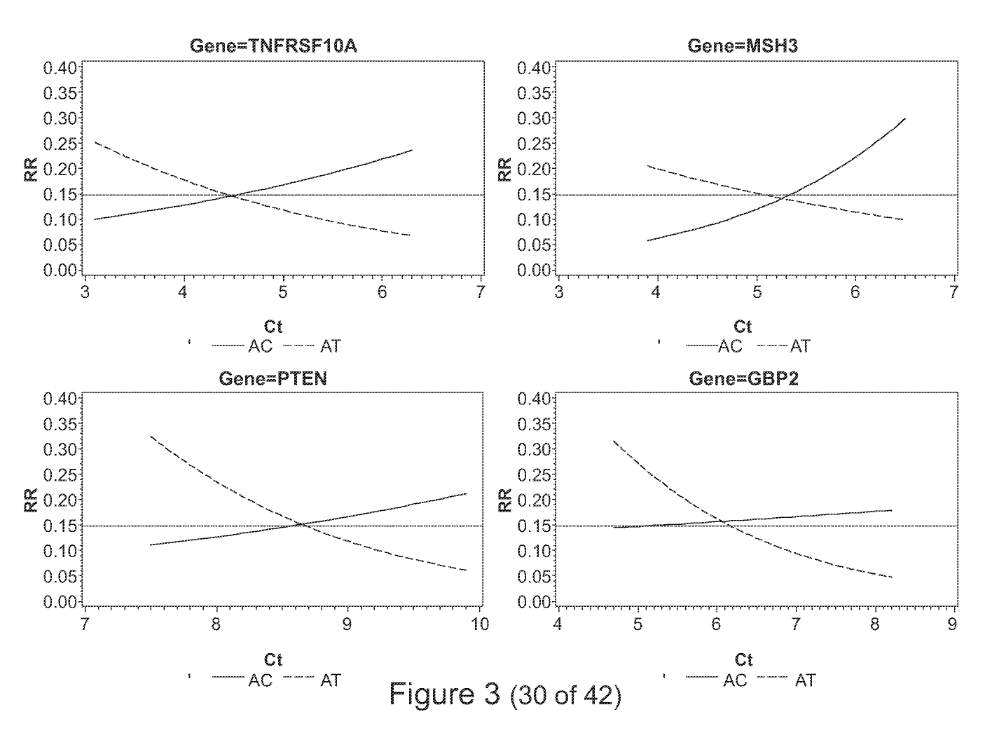

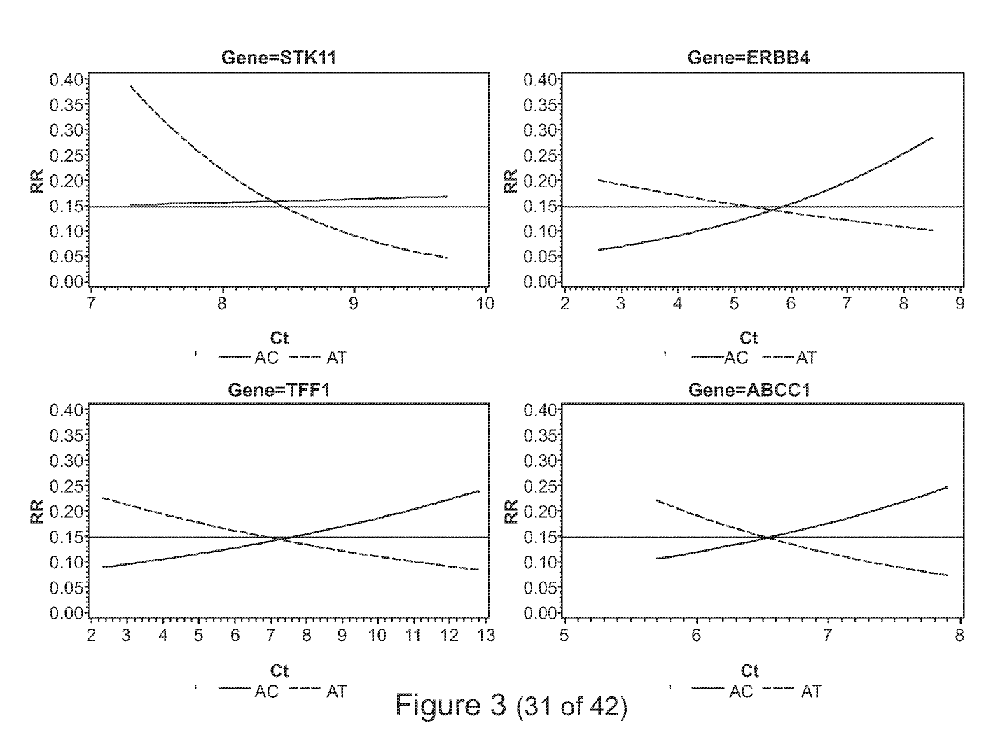

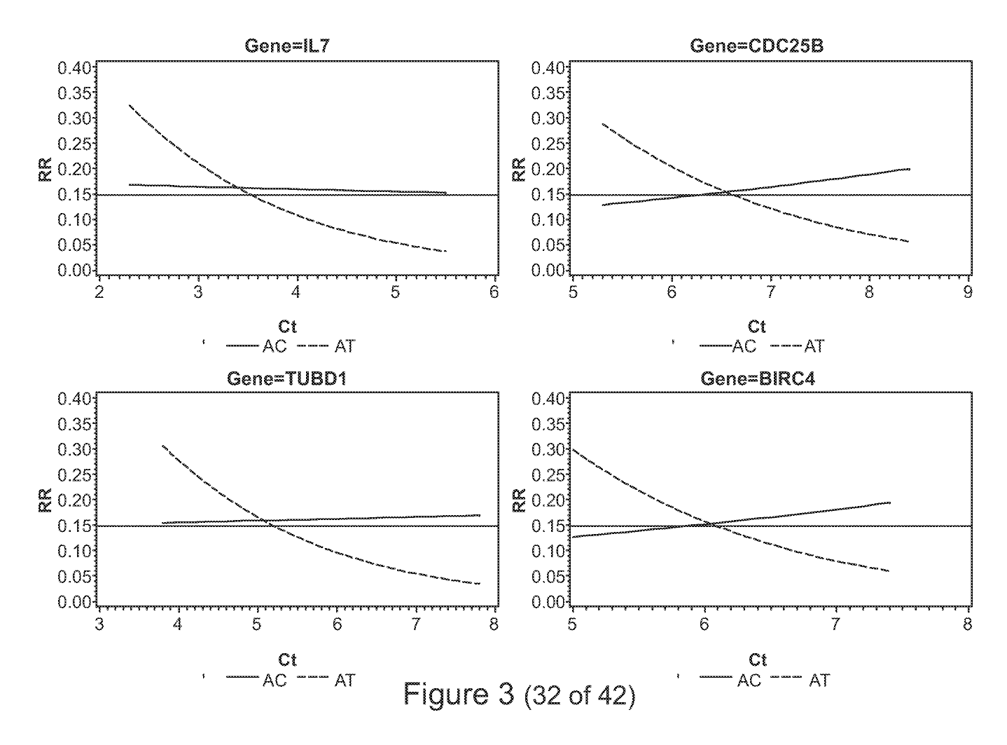

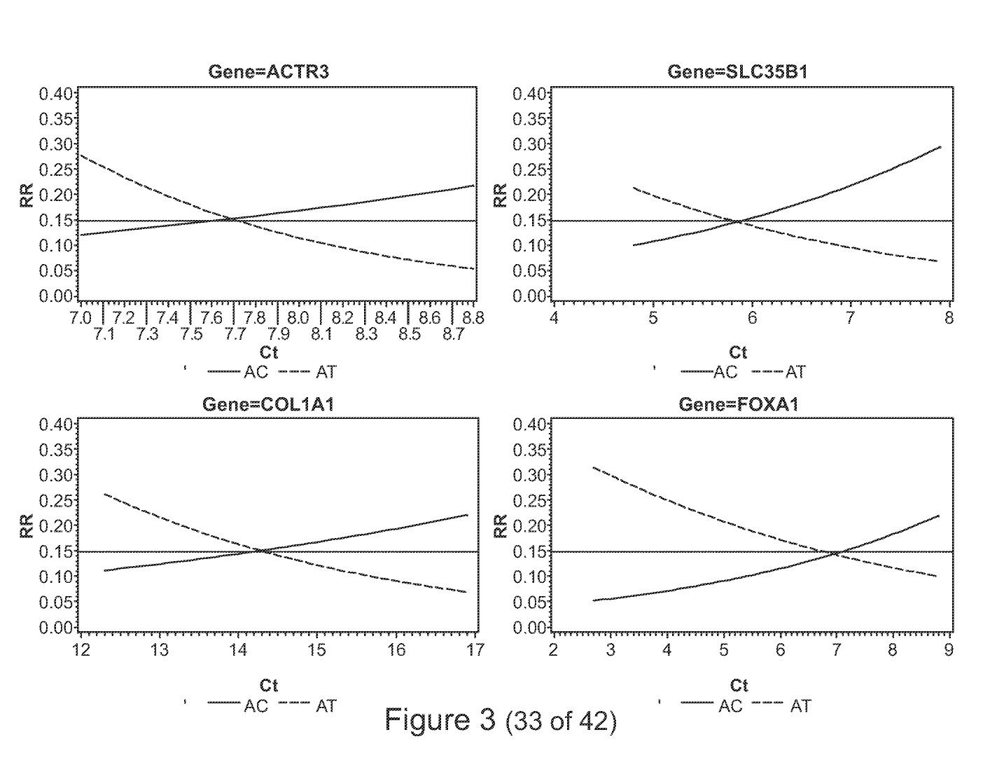

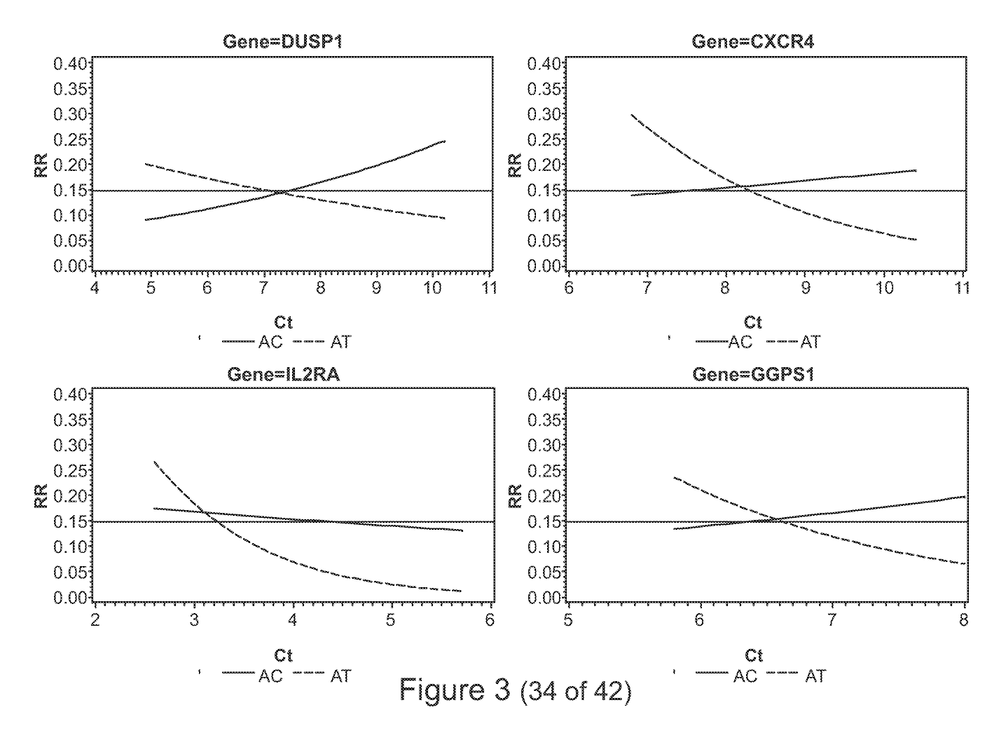

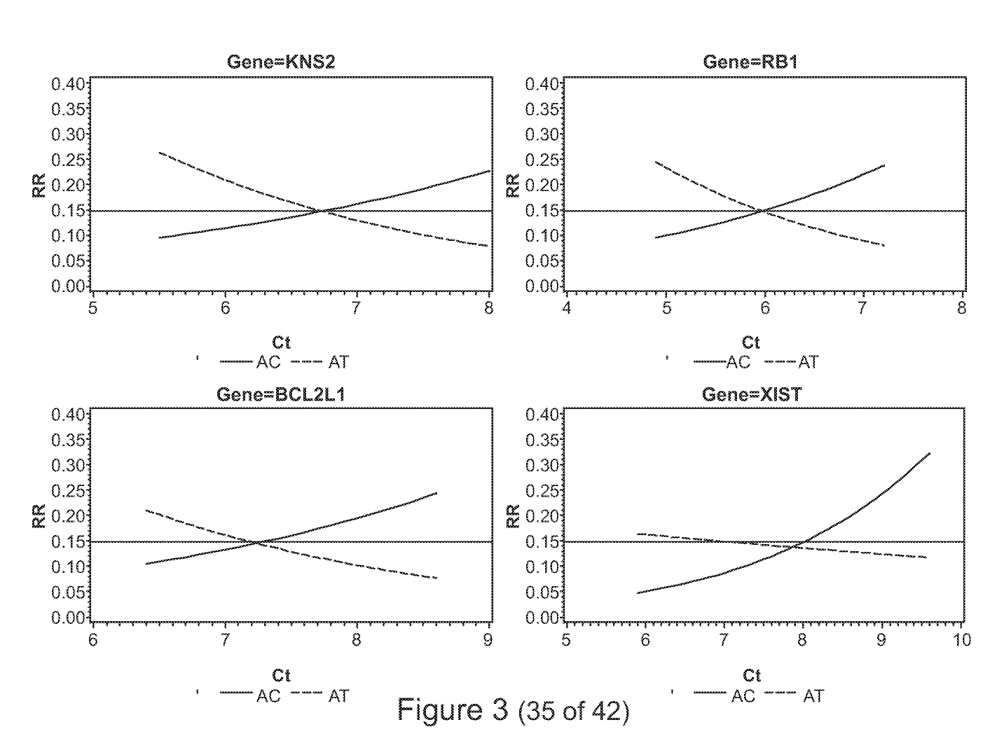

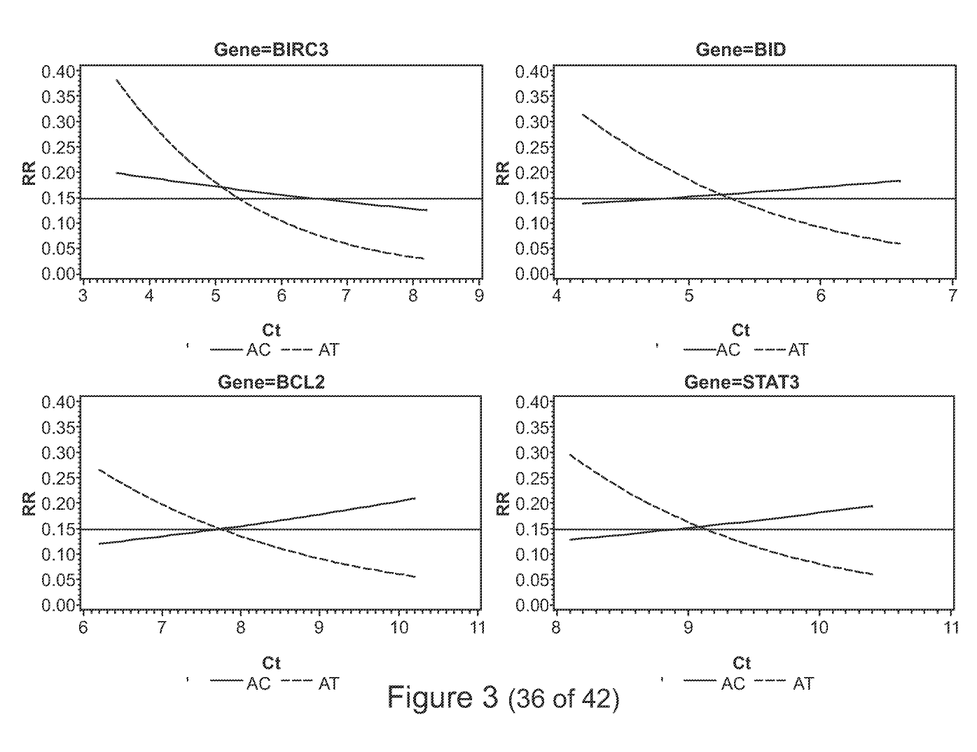

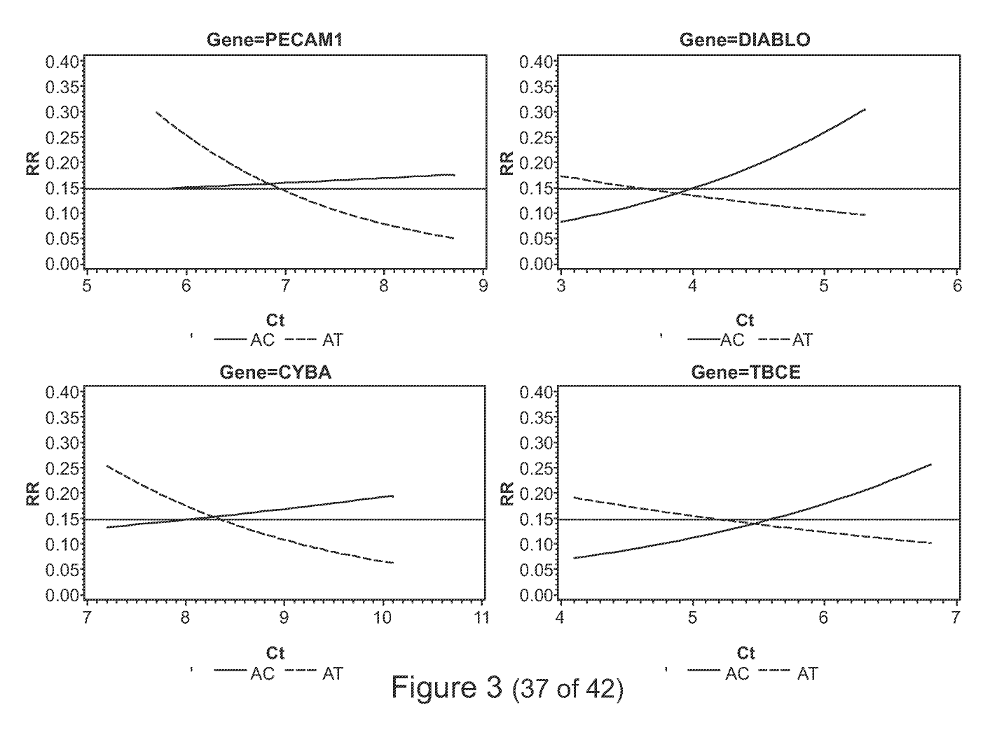

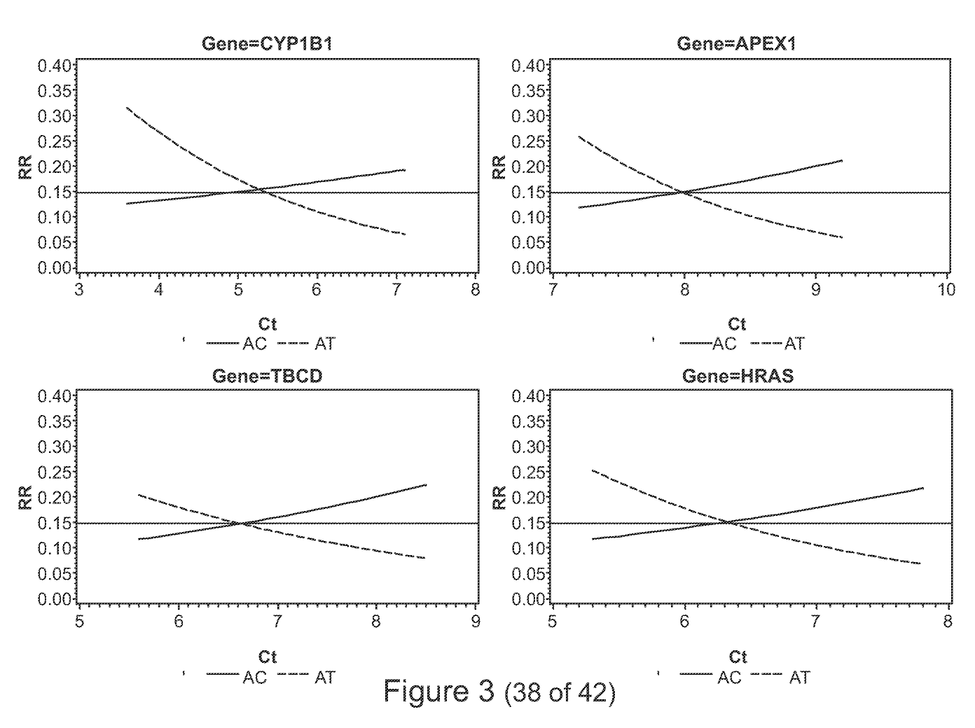

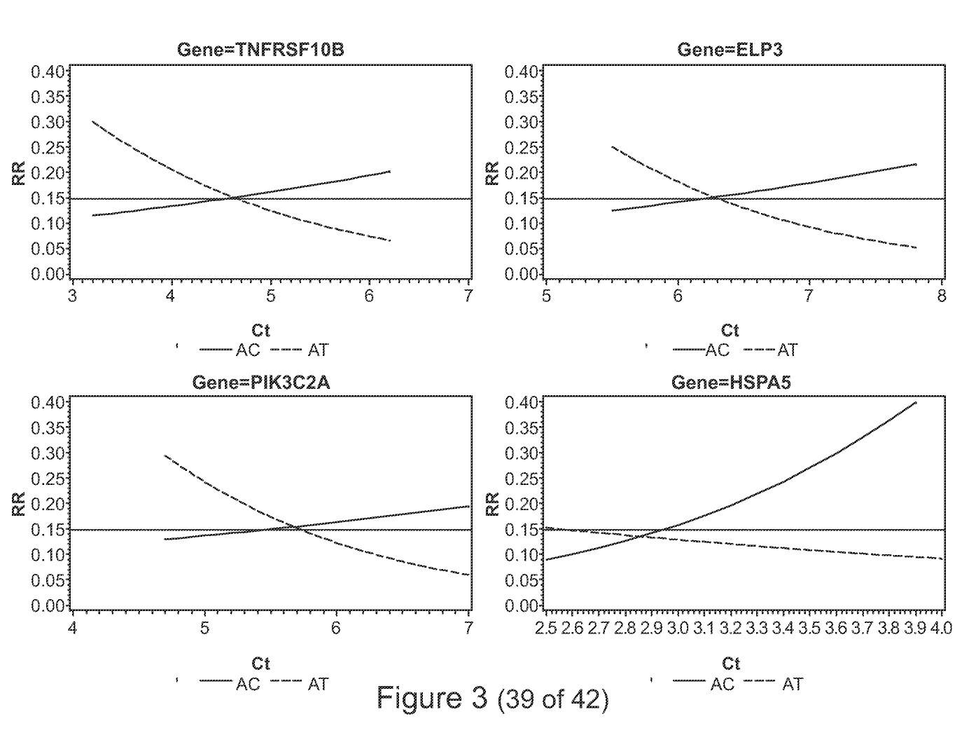

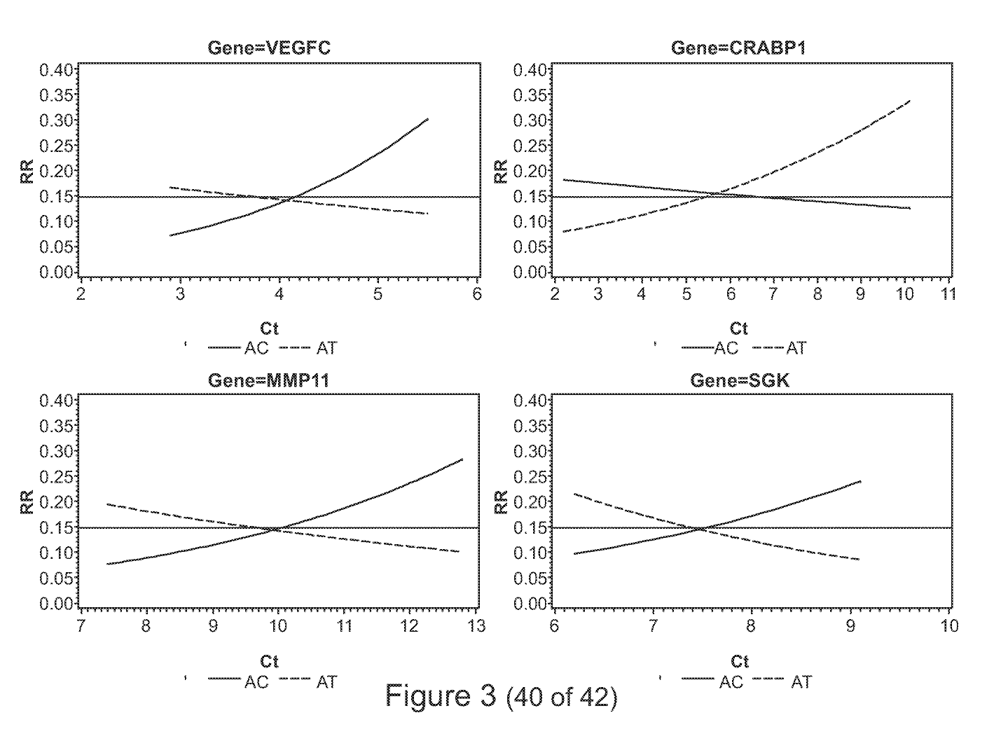

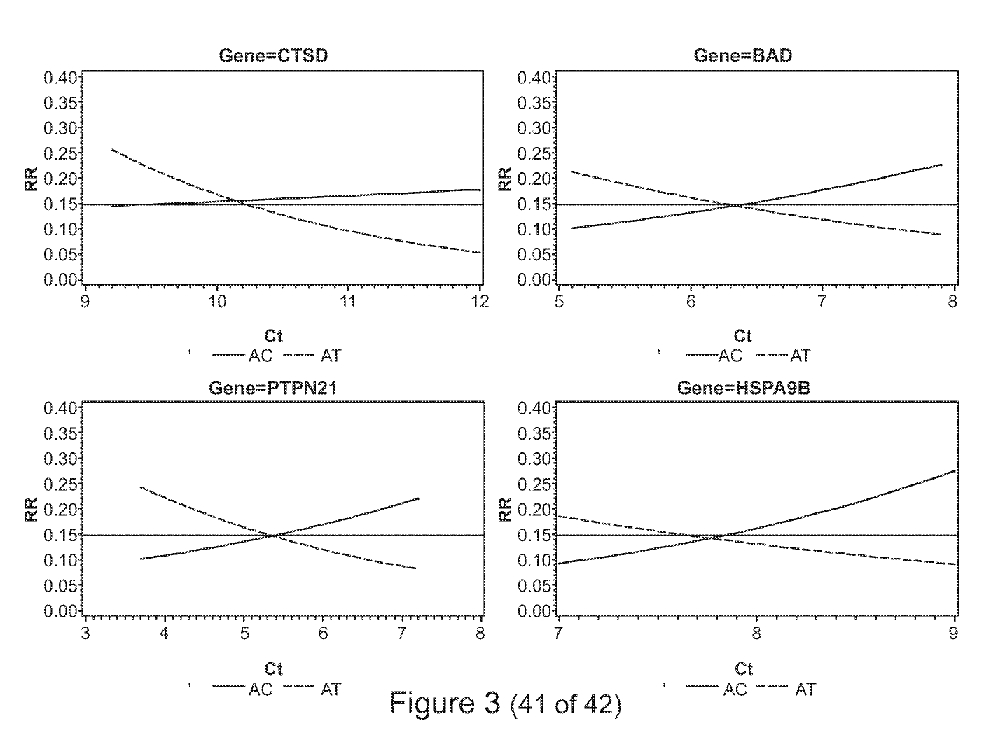

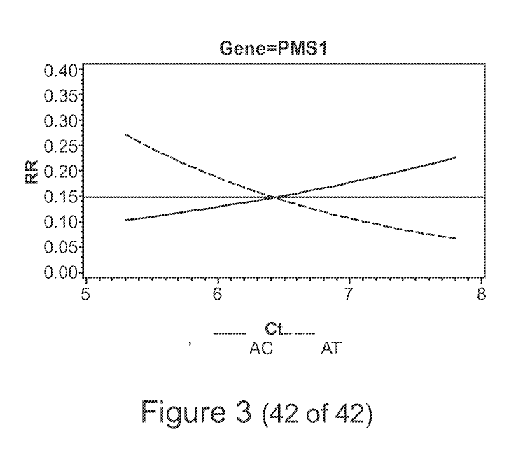

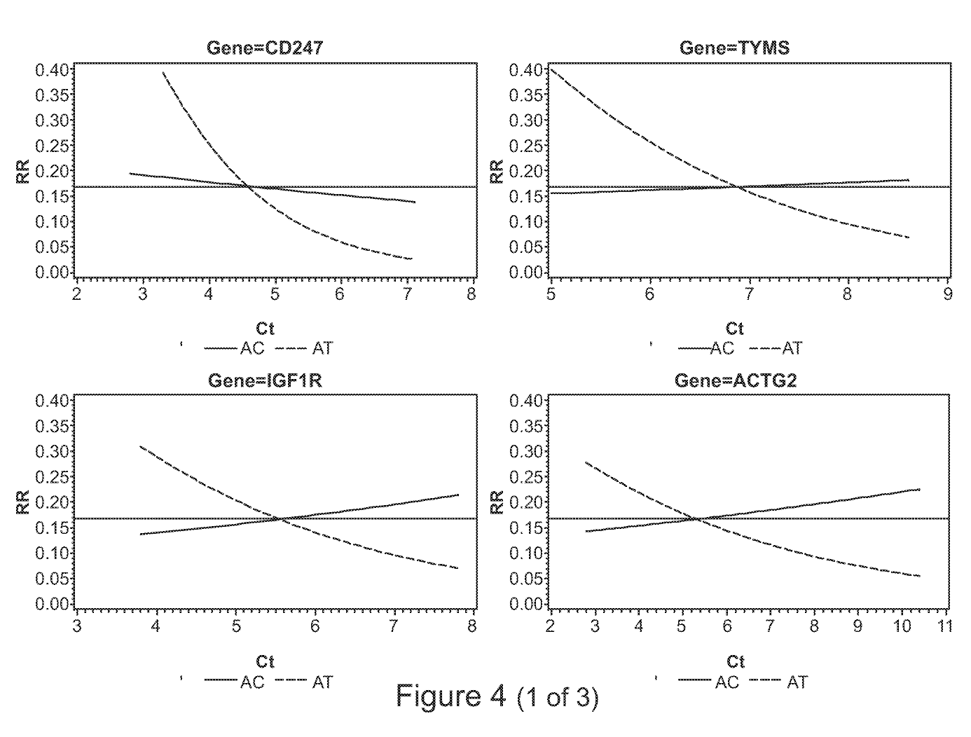

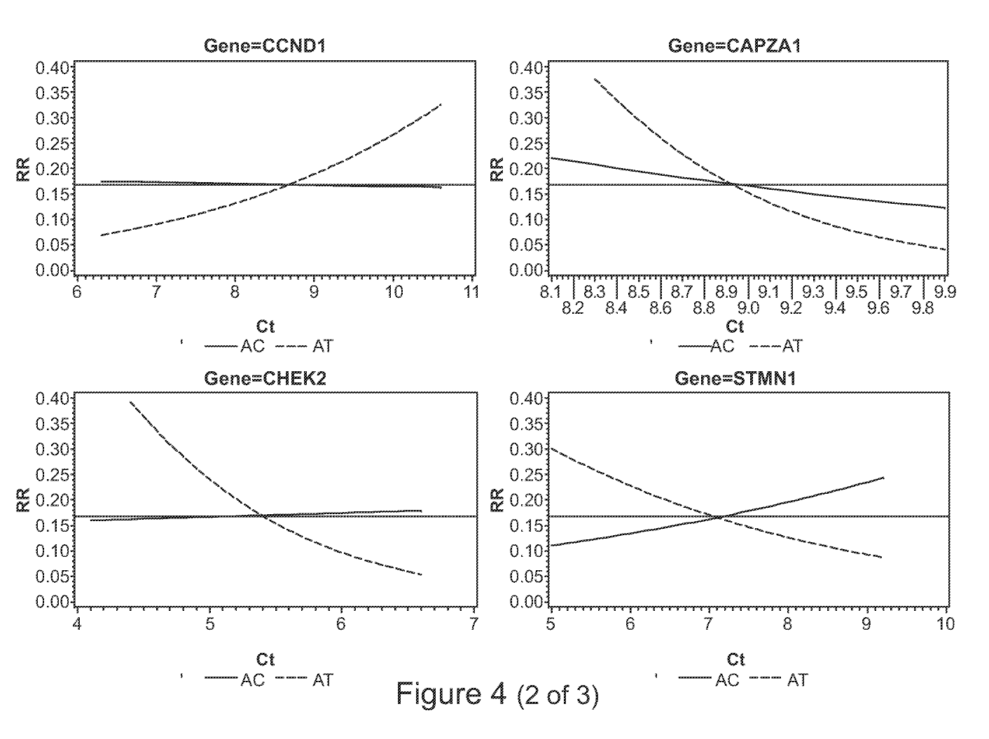

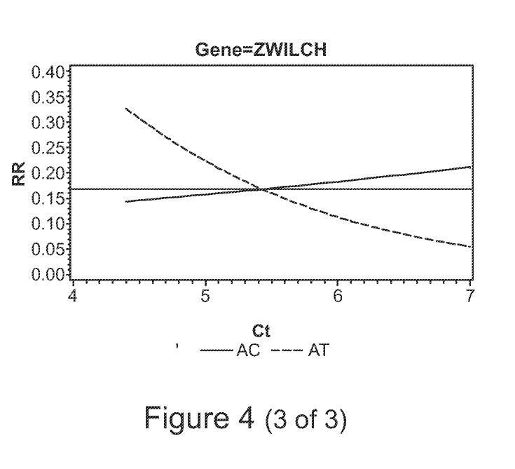

Other characteristics of the tumor can be taken into account when assessing likelihood of taxane and/or cyclophosphamide benefit by analysis of expression level of a marker gene disclosed herein. For example, hormone receptor expression status (e.g., ER⁺, ER⁻, PR⁺, PR⁻) can be assessed for the tumor sample, and taken into consideration when evaluating expression levels of the marker gene, e.g., the expression level is compared to expression level correlations to TB and/or CB in a population sharing the same characteristics. For example, FIG. 1 provides TB (AT) and CB (AC) prediction curves in all patients in the study discussed in the Examples below without regard to hormone expression status or likelihood of cancer recurrence as predicted by the Oncotype DX RS. FIG. 2 provides TB (AT) and CB (AC) prediction curves in hormone receptor positive patients. FIG. 3 provides TB (AT) and CB (AC) prediction curves in hormone receptor positive patients having an Oncotype DX RS score of about 18 or greater, which indicates a significant risk of cancer recurrence within 10 years following surgery and tamoxifen therapy. FIG. 4 provides TB (AT) and CB (AC) prediction curves in hormone receptor negative patients.

The prediction curves can be used to assess information provided by an expression level of a marker gene disclosed herein and in turn facilitate a treatment decision with respect to selection of a taxane-containing and/or a cyclophosphamide-containing regimen. For example, where a gene exhibits an expression level having a TB (AT) prediction curve having a negative slope as exemplified in FIGS. 1-4, then increasing normalized expression levels of the gene are positively correlated with a likelihood of clinical benefit of including a taxane in the treatment regimen (since patients who exhibited this expression pattern of the particular gene had lower recurrence rates following a taxane-containing regimen). Conversely, where a gene exhibits an expression level having a TB (AT) prediction curve having a positive slope as exemplified in FIGS. 1-4, then increasing normalized expression levels of the gene are negatively correlated with a likelihood of clinical benefit of including a taxane in the treatment regimen. Similarly, where a gene exhibits an expression level having a CB (AC) prediction curve having a negative slope as exemplified in FIGS. 1-4, then increasing normalized expression levels of the gene are positively correlated with a likelihood of clinical benefit of including a cyclophosphamide in the treatment regimen (since patients who exhibited this expression pattern of the particular gene had lower recurrence rates following cyclophosphamide-containing regimen). Conversely, where a gene exhibits an expression level having a CB (AC) prediction curve having a positive slope as exemplified in FIGS. 1-4, then increasing normalized expression levels of the gene are negatively correlated with a likelihood of clinical benefit of including a cyclophosphamide in the treatment regimen.

Accordingly, the expression levels of the marker genes can be used to facilitate a decision as to whether a taxane should be included or excluded in a treatment regimen, and to facilitate a decision as to whether a cyclophosphamide should be included or excluded in a treatment regimen. The marker genes can be used to facilitate selection of a treatment regimen that includes, a taxane and/or a cyclophosphamide, or neither a taxane nor a cyclophosphamide.

In some instances the marker gene expression level may suggest clinical benefit for both a taxane and a cyclophosphamide, e.g., where increasing expression levels are associated with a recurrence risk below a selected recurrence risk. For example, as illustrated in FIG. 2 for the gene ZW10, increased expression of ZW10 in HR-positive cancer patients is associated with increased likelihood of clinical benefit for both a taxane and for a cyclophosphamide. In addition, because the magnitudes of the slopes are significantly different, patients with increased expression of ZW10 are predicted to have lower risks of recurrence if treated with AT instead of AC, and patients with decreased expression of ZW10 are predicted to have lower risks of recurrence if treated with AC instead of AT. Thus, the marker genes that are associated with TB (AT) and CT (AC) prediction curves that differ in slope can facilitate a decision in selecting between a taxane-containing regimen and a cyclophosphamide-containing regimen, even where there may be clinical benefit with either or both treatment regimen.

The methods of the present disclosure also can facilitate selection between a taxane-containing regimen and a cyclophosphamide-containing regimen (e.g., between and AT and AC therapy). For example, where the curves in FIGS. 1-4 have significantly different slopes in the Cox regression model and the TB (AT) and CB (AC) prediction curves cross, expression levels of the marker gene can be used to assess the likelihood the patient will respond to a taxane-containing regimen (e.g., AT) or to a cyclophosphamide-containing regimen (e.g., AC).

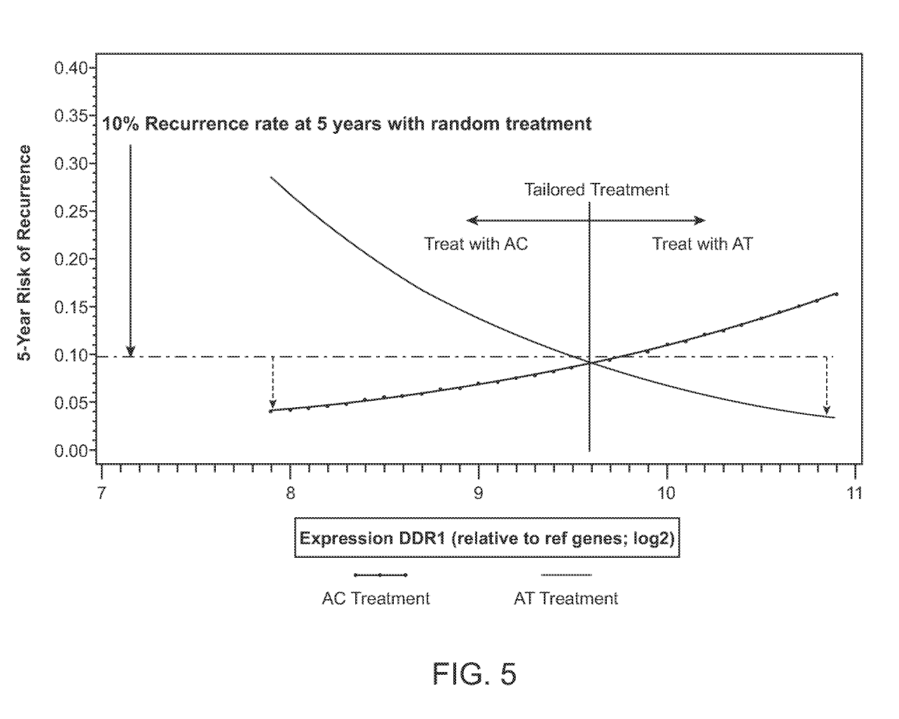

For example, FIG. 5 illustrates a plot of the 5-year risk of relapse versus gene expression, presented for an exemplary gene, DDR1. As illustrated in FIG. 5, the expression level of DDR1 can be used to facilitate selection of therapy where treatment with a cyclophosphamide is favored over treatment with a taxane at lower expression levels of DDR1, with a “switch” of the relative clinical benefit of these therapies occurring at a point where the recurrence risk associated with taxane treatment is lower than that associated with cyclophosphamide treatment, thus favoring a treatment regimen including a taxane over a cyclophosphamide.

There are many types of systemic treatment regimens available for patients diagnosed with cancer. For example, the table below lists various chemotherapeutic and hormonal therapies for breast cancer.

### Single Agents Useful in Breast Cancer

| | COMMON | |
|---|---|---|
| GENERIC NAME | TRADE NAME | CLASS |
| Cyclophosphamide (C) | Cytoxan ® | Nitrogen mustards |
| Doxorubicin | Adriamycin ® | Anthracyclines |
| Epirubicin | Pharmorubicin ® | Anthracyclines |
| Fluorouracil | | Pyrimidine analogs |
| Methotrexate | Rheumatrex ® | Folic acid analogs |
| Paclitaxel | Taxol ® | Taxanes (T) |
| Docetaxel | Taxotere ® | Taxanes (T) |
| Capecitabine | Xeloda ® | Pyrimidine analogs |
| Trastuzumab | Herceptin ® | Monoclonal Antibodies |
| Bevacizumab | Avastin ® | Monoclonal Antibodies |

### Combinations Useful in Breast Cancer

| | | |
|---|---|---|
| CAF | Cyclophosphamide, Adriamycin, Fluorouracil | US |
| CMF | Cyclophosphamide, Methotrexate, Fluorouracil | US |
| AC | Adriamycin, Cyclophosphamide | US |
| AT | Adriamycin, Taxane | US |
| ACT | Adriamycin, Cyclophosphamide, Taxane | US |
| TAC | Taxane, Adriamycin, Cyclophosphamide | US |
| TC | Taxane, Cyclophosphamide | US |
| | Fluorouracil, Epirubicin, Cyclophosphamide | Europe |

### Gene Expression Profiling

The practice of the methods and compositions of the present disclosure will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

Methods of gene expression profiling include methods based on hybridization analysis of polynucleotides, methods based on sequencing of polynucleotides, and proteomics-based methods. Exemplary methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription PCT (RT-PCR) (Weis et al., Trends in Genetics 8:263-264 (1992)). Antibodies may be employed that can recognize sequence-specific duplexes, including DNA duplexes, RNA duplexes, and DNA-RNA hybrid duplexes or DNA-protein duplexes. Representative methods for nucleic acid sequencing analysis include Serial Analysis of Gene Expression (SAGE), and Digital Gene Expression (DGE).

Representative methods of gene expression profiling are disclosed, for example, in U.S. Pat. Nos. 7,056,674 and 7,081,340, and in U.S. Patent Publication Nos. 20020095585; 20050095634; 20050260646; and 20060008809. Representative scientific publications including methods of gene expression profiling, including data analysis, include Gianni et al., J Clin Oncol. 2005 Oct. 10; 23(29):7265-77; Paik et al., N Engl J Med. 2004 Dec. 30; 351(27):2817-26; and Cronin et al., Am J Pathol. 2004 January; 164(1):35-42. The disclosures of these patent and scientific publications are expressly incorporated by reference herein.

### Reverse Transcriptase PCR (RT-PCR)

Typically, mRNA is isolated from a test sample. The starting material is typically total RNA isolated from a human tumor, usually from a primary tumor. Optionally, normal tissues from the same patient can be used as an internal control. mRNA can be extracted from a tissue sample, e.g., from a sample that is fresh, frozen (e.g. fresh frozen), or paraffin-embedded and fixed (e.g. formalin-fixed).

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The sample containing the RNA is then subjected to reverse transcription to produce cDNA from the RNA template, followed by exponential amplification in a PCR reaction. The two most commonly used reverse transcriptase enzymes are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

PCR-based methods use a thermostable DNA-dependent DNA polymerase, such as a Taq DNA polymerase. For example, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction product. A third oligonucleotide, or probe, can be designed to facilitate detection of a nucleotide sequence of the amplicon located between the hybridization sites the two PCR primers. The probe can be detectably labeled, e.g., with a reporter dye, and can further be provided with both a fluorescent dye, and a quencher fluorescent dye, as in a Taqman® probe configuration. Where a Taqman® probe is used, during the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as a threshold cycle (“Cₜ”). Fluorescence values are recorded during every cycle and represent the amount of product amplified to that point in the amplification reaction. The threshold cycle (Cₜ) is generally described as the point when the fluorescent signal is first recorded as statistically significant.

It is desirable to correct for (normalize away) both differences in the amount of RNA assayed and variability in the quality of the RNA used. Therefore, the assay typically measures, and expression analysis of a marker gene incorporates analysis of, the expression of certain reference genes (or “normalizing genes”), including well known housekeeping genes, such as GAPDH. Alternatively, normalization can be based on the mean or median signal (Ct) of all of the assayed genes or a large subset thereof (often referred to as a “global normalization” approach). On a gene-by-gene basis, measured normalized amount of a patient tumor mRNA may be compared to the amount found in a colon cancer tissue reference set. See M. Cronin, et al., Am. Soc. Investigative Pathology 164:35-42 (2004).

Gene expression measurements can be normalized relative to the mean of one or more (e.g., 2, 3, 4, 5, or more) reference genes. Reference-normalized expression measurements can range from 0 to 15, where a one unit increase generally reflects a 2-fold increase in RNA quantity.

RT-PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for use in the methods of the present disclosure use fixed, paraffin-embedded tissues as the RNA source mRNA isolation, purification, primer extension and amplification can be preformed according to methods available in the art. (see, e.g., Godfrey et al. J. Molec. Diagnostics 2: 84-91 (2000); Specht et al., Am. J. Pathol. 158: 419-29 (2001)). Briefly, a representative process starts with cutting about 10 μm thick sections of paraffin-embedded tumor tissue samples. The RNA is then extracted, and protein and DNA depleted from the RNA-containing sample. After analysis of the RNA concentration, RNA is reverse transcribed using gene specific primers followed by RT-PCR to provide for cDNA amplification products.

Design of Intron-Based PCR Primers and Probes

PCR primers and probes can be designed based upon exon or intron sequences present in the mRNA transcript of the gene of interest. Primer/probe design can be performed using publicly available software, such as the DNA BLAT software developed by Kent, W. J., Genome Res. 12(4):656-64 (2002), or by the BLAST software including its variations.

Where necessary or desired, repetitive sequences of the target sequence can be masked to mitigate non-specific signals. Exemplary tools to accomplish this include the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Rrawetz et al. (eds.) Bioinformatics Methods and Protocols: Methods in Molecular Biology. Humana Press, Totowa, N.J., pp 365-386).

Other factors that can influence PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, and exhibit Tm's between 50 and 80° C., e.g. about 50 to 70° C.

For further guidelines for PCR primer and probe design see, e.g. Dieffenbach, C W. et al, “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

Quantitative PCR for Gene Expression Analysis

Per VanGuilder et al., BioTechniques 44: 619 (2008), quantitative PCR (qPCR) now represents the method of choice for analyzing gene expression of numerous genes in anywhere from a small number to thousands of samples. For investigators studying gene expression, there is a multitiered technological approach depending on the number of genes and samples being examined. Gene expression microarrays are still the preferred method for large-scale (e.g., whole-genome) discovery experiments. Due to the logistics, sensitivity, and costs of whole-genome micorarrays, there is also a niche for focused microarrays that allow for analysis of a smaller number of genes in a larger number of samples. Nonetheless, for validation of microarray discovery, reverse-transcription quantitative PCR (RT-qPCR) remains the gold standard. The current maturation of real-time qPCR with fluorescent probes allows for rapid and easy confirmation of microarray results in a large number of samples. Often, a whole-genome discovery experiment is not required, as the gene or pathway of interest is already known. In that case, the data collection can begin with qPCR. Finally, qPCR has also shown great utility in biomarker monitoring. In this scenario, previously developed identified targets can be assayed in very large numbers of samples (1000s).

Data Analysis. Analysis of real-time qPCR data has also reached a mature stage of development. Analyses can be either of absolute levels (i.e., numbers of copies of a specific RNA per sample) or relative levels (i.e., sample 1 has twice as much mRNA of a specific gene as sample 2). By far, the majority of analyses use relative quantitation as this is easier to measure and is of primary interest to researchers examining disease states. For absolute quantitation, an RNA standard curve of the gene of interest is required in order to calculate the number of copies. In this case, a serial dilution of a known amount (number of copies) of pure RNA is diluted and subjected to amplification. Like a protein assay, the unknown signal is compared with the curve so as to extrapolate the starting concentration.

The most common method for relative quantitation is the 2^{-ΔΔCT} method. This method relies on two assumptions. The first is that the reaction is occurring with 100% efficiency; in other words, with each cycle of PCR, the amount of product doubles. This can be ascertained through simple experiments as described in the scientific literature. This assumption is also one of the reasons for using a low cycle number when the reaction is still in the exponential phase. In the initial exponential phase of PCR, substrates are not limiting and there is no degradation of products. In practice, this requires setting the crossing threshold or cycle threshold (Cₜ) at the earliest cycle possible. The Cₜ is the number of cycles that it takes each reaction to reach an arbitrary amount of fluorescence. The second assumption of the 2^{-ΔΔCT} method is that there is a gene (or genes) that is expressed at a constant level between the samples. This endogenous control will be used to correct for any difference in sample loading.

Once the Cₜ value is collected for each reaction, it can be used to generate a relative expression level. One 2^{-ΔΔCT} method is now described. In this example, there are two samples (Control and Treated) and we have measured the levels of (i) a gene of interest (Target Gene (TG)) and (ii) an endogenous control gene (Control Gene (CG)). For each sample, the difference in Cₜ values for the gene of interest and the endogenous control is calculated (the ΔCₜ). Next, subtraction of the control-condition ΔCₜ from the treated-condition ΔCₜ yields the ΔΔCₜ. The negative value of this subtraction, the −ΔΔCₜ, is used as the exponent of 2 in the equation and represents the difference in “corrected” number of cycles to threshold. The exponent conversion comes from the fact that the reaction doubles the amount of product per cycle. For example, if the control sample ΔCₜ is 2 and the treated sample ΔCₜ is 4, computing the 2^{-ΔΔCT} (which becomes 2⁻⁽⁴⁻²⁾) yields 0.25. This value is often referred to as the RQ, or relative quantity value. This means that the level of the gene of interest in the treated sample is only 25% of the level of that gene in the control sample. This becomes evident because the treated sample took two more cycles of PCR to reach the same amount of product as the control sample and therefore there was less of that cDNA to begin with in the treated sample. The 2^{-ΔΔCT} method is the most common quantitation strategy, but it should be noted that there are other valid methods for analyzing qPCR Cₜ values. Several investigators have proposed alternative analysis methods.

MassARRAY® System

In MassARRAY-based methods, such as the exemplary method developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

Other PCR-Based Methods

Further PCR-based techniques that can find use in the methods disclosed herein include, for example, BeadArray® technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression® (BADGE), using the commercially available Luminex 100 LabMAP® system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003).

Microarrays

Expression levels of a gene of interest can also be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from mRNA of a test sample. As in the RT-PCR method, the source of mRNA typically is total RNA isolated from a tumor sample, and optionally from normal tissue of the same patient as an internal control or cell lines. mRNA can be extracted, for example, from frozen or archived paraffin-embedded and fixed (e.g. formalin-fixed) tissue samples.

For example, PCR amplified inserts of cDNA clones of a gene to be assayed are applied to a substrate in a dense array. Usually at least 10,000 nucleotide sequences are applied to the substrate. For example, the microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After washing under stringent conditions to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et at, Proc. Natl. Acad. Sci. USA 93(2):106-149 (1996)). Microarray analysis can be performed by commercially available equipment, following manufacturer's protocols, such as by using the Affymetrix GenChip® technology.

Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

Gene Expression Analysis by Nucleic Acid Sequencing

Nucleic acid sequencing technologies are suitable methods for analysis of gene expression. The principle underlying these methods is that the number of times a cDNA sequence is detected in a sample is directly related to the relative expression of the mRNA corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). More recently, the advent of “next-generation” sequencing technologies has made DGE simpler, higher throughput, and more affordable. As a result, more laboratories are able to utilize DGE to screen the expression of more genes in more individual patient samples than previously possible. See, e.g., J. Marioni, Genome Research 18(9):1509-1517 (2008); R. Morin, Genome Research 18(4):610-621 (2008); A. Mortazavi, Nature Methods 5(7):621-628 (2008); N. Cloonan, Nature Methods 5(7):613-619 (2008).

Isolating RNA from Body Fluids

Methods of isolating RNA for expression analysis from tissue (e.g., breast tissue), blood, plasma and serum (See for example, Tsui N B et al. (2002) 48, 1647-53 and references cited therein) and from urine (See for example, Boom R et al. (1990) J Clin Microbiol. 28, 495-503 and reference cited therein) have been described.

Immunonological Methods

Immunological methods (e.g., immunohistochemistry methods) are also suitable for detecting the expression levels of genes and applied to the method disclosed herein. Antibodies (e.g., monoclonal antibodies) that specifically bind a gene product of a gene of interest can be used in such methods. The antibodies can be detected by direct labeling of the antibodies themselves, for example, with radioactive labels, fluorescent labels, haptene labels such as, biotin, or an enzyme such as horse radish peroxidase or alkaline phosphatase. Alternatively, unlabeled primary antibody can be used in conjunction with a labeled secondary antibody specific for the primary antibody. Immunological methods protocols and kits are well known in the art and are commercially available.

Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics.

### General Description of Exemplary Protocol

The steps of a representative protocol for profiling gene expression using fixed, paraffin-embedded tissues as the RNA source, including mRNA isolation, purification, primer extension and amplification are provided in various published journal articles. (See, e.g., T. E. Godfrey et al., J. Molec. Diagnostics 2: 84-91 (2000); K. Specht et al., Am. J. Pathol. 158: 419-29 (2001), M. Cronin, et al., Am J Pathol 164:35-42 (2004)). Briefly, a representative process starts with cutting a tissue sample section (e.g. about 10 μm thick sections of a paraffin-embedded tumor tissue sample). The RNA is then extracted, and protein and DNA are removed. After analysis of the RNA concentration, RNA repair is performed if desired. The sample can then be subjected to analysis, e.g., by reverse transcribed using gene specific promoters followed by RT-PCR.

### Kits

The materials for use in the methods of the present disclosure are suited for preparation of kits produced in accordance with well known procedures. The present disclosure thus provides kits comprising agents, which may include gene-specific or gene-selective probes and/or primers, for quantitating the expression of the disclosed genes for predicting clinical outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present disclosure. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present disclosure (e.g., appropriate length poly(T) or random primers linked to a promoter reactive with the RNA polymerase). Mathematical algorithms used to estimate or quantify prognostic and/or predictive information are also properly potential components of kits.

The methods provided by the present disclosure may also be automated in whole or in part.

### Reports

The methods of the present disclosure are suited for the preparation of reports summarizing the predictions resulting from the methods of the present disclosure. A “report,” as described herein, is an electronic or tangible document which includes report elements that provide information of interest relating to a likelihood assessment and its results. A subject report includes at least a likelihood assessment, e.g., an indication as to the likelihood that a cancer patient will exhibit a beneficial clinical response to a treatment regimen of interest. A subject report can be completely or partially electronically generated, e.g., presented on an electronic display (e.g., computer monitor). A report can further include one or more of: 1) information regarding the testing facility; 2) service provider information; 3) patient data; 4) sample data; 5) an interpretive report, which can include various information including: a) indication; b) test data, where test data can include a normalized level of one or more genes of interest, and 6) other features.

The present disclosure thus provides for methods of creating reports and the reports resulting therefrom. The report may include a summary of the expression levels of the RNA transcripts, or the expression products of such RNA transcripts, for certain genes in the cells obtained from the patients tumor tissue. The report may include a prediction that said subject has an increased likelihood of response to treatment with a particular chemotherapy or the report may include a prediction that the subject has a decreased likelihood of response to the chemotherapy. The report may include a recommendation for treatment modality such as surgery alone or surgery in combination with chemotherapy. The report may be presented in electronic format or on paper.

Thus, in some embodiments, the methods of the present disclosure further includes generating a report that includes information regarding the patient's likelihood of response to chemotherapy, particularly a therapy including cyclophophamide and/or a taxane. For example, the methods disclosed herein can further include a step of generating or outputting a report providing the results of a subject response likelihood assessment, which report can be provided in the form of an electronic medium (e.g., an electronic display on a computer monitor), or in the form of a tangible medium (e.g., a report printed on paper or other tangible medium).

A report that includes information regarding the likelihood that a patient will respond to treatment with chemotherapy, particularly a including cyclophophamide and/or a taxane, is provided to a user. An assessment as to the likelihood that a cancer patient will respond to treatment with chemotherapy, or predicted comparative response to two therapy options, is referred to below as a “response likelihood assessment” or, simply, “likelihood assessment.” A person or entity who prepares a report (“report generator”) will also perform the likelihood assessment. The report generator may also perform one or more of sample gathering, sample processing, and data generation, e.g., the report generator may also perform one or more of: a) sample gathering; b) sample processing; c) measuring a level of an indicator response gene product(s); d) measuring a level of a reference gene product(s); and e) determining a normalized level of a response indicator gene product(s). Alternatively, an entity other than the report generator can perform one or more sample gathering, sample processing, and data generation.

For clarity, it should be noted that the term “user,” which is used interchangeably with “client,” is meant to refer to a person or entity to whom a report is transmitted, and may be the same person or entity who does one or more of the following: a) collects a sample; b) processes a sample; c) provides a sample or a processed sample; and d) generates data (e.g., level of a response indicator gene product(s); level of a reference gene product(s); normalized level of a response indicator gene product(s)) for use in the likelihood assessment. In some cases, the person(s) or entity(ies) who provides sample collection and/or sample processing and/or data generation, and the person who receives the results and/or report may be different persons, but are both referred to as “users” or “clients” herein to avoid confusion. In certain embodiments, e.g., where the methods are completely executed on a single computer, the user or client provides for data input and review of data output. A “user” can be a health professional (e.g., a clinician, a laboratory technician, a physician (e.g., an oncologist, surgeon, pathologist), etc.).

In embodiments where the user only executes a portion of the method, the individual who, after computerized data processing according to the methods of the invention, reviews data output (e.g., results prior to release to provide a complete report, a complete, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

Where government regulations or other restrictions apply (e.g., requirements by health, malpractice, or liability insurance), all results, whether generated wholly or partially electronically, are subjected to a quality control routine prior to release to the user.

### Computer-Based Systems and Methods

The methods and systems described herein can be implemented in numerous ways. In one embodiment of particular interest, the methods involve use of a communications infrastructure, for example the internet. Several embodiments of the invention are discussed below. It is also to be understood that the present invention may be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site associated (e.g., at a service provider's facility).

For example, during or after data input by the user, portions of the data processing can be performed in the user-side computing environment. For example, the user-side computing environment can be programmed to provide for defined test codes to denote a likelihood “score,” where the score is transmitted as processed or partially processed responses to the reviewer's computing environment in the form of test code for subsequent execution of one or more algorithms to provide a results and/or generate a report in the reviewer's computing environment. The score can be a numerical score (representative of a numerical value) or a non-numerical score representative of a numerical value or range of numerical values (e.g., “A’ representative of a 90-95% likelihood of an outcome; “high” representative of a greater than 50% chance of response (or some other selected threshold of likelihood); “low” representative of a less than 50% chance of response (or some other selected threshold of likelihood); and the like.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which can include test data (e.g., level of a response indicator gene product(s); level of a reference gene product(s); normalized level of a response indicator gene product(s)); and may also include other data such as patient data. This information received can be stored at least temporarily in a database, and data analyzed to generate a report as described above.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In an embodiment of particular interest, all or a portion of the input data and/or all or a portion of the output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record which may exist in a confidential database at the healthcare facility.

A system for use in the methods described herein generally includes at least one computer processor (e.g., where the method is carried out in its entirety at a single site) or at least two networked computer processors (e.g., where data is to be input by a user (also referred to herein as a “client”) and transmitted to a remote site to a second computer processor for analysis, where the first and second computer processors are connected by a network, e.g., via an intranet or internet). The system can also include a user component(s) for input; and a reviewer component(s) for review of data, generated reports, and manual intervention. Additional components of the system can include a server component(s); and a database(s) for storing data (e.g., as in a database of report elements, e.g., interpretive report elements, or a relational database (RDB) which can include data input by the user and data output. The computer processors can be processors that are typically found in personal desktop computers (e.g., IBM, Dell, Macintosh), portable computers, mainframes, minicomputers, or other computing devices.

The networked client/server architecture can be selected as desired, and can be, for example, a classic two or three tier client server model. A relational database management system (RDMS), either as part of an application server component or as a separate component (RDB machine) provides the interface to the database.

In one example, the architecture is provided as a database-centric client/server architecture, in which the client application generally requests services from the application server which makes requests to the database (or the database server) to populate the report with the various report elements as required, particularly the interpretive report elements, especially the interpretation text and alerts. The server(s) (e.g., either as part of the application server machine or a separate RDB/relational database machine) responds to the client's requests.

The input client components can be complete, stand-alone personal computers offering a full range of power and features to run applications. The client component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe and offers data management, information sharing between clients, network administration and security. The application and any databases used can be on the same or different servers.

Other computing arrangements for the client and server(s), including processing on a single machine such as a mainframe, a collection of machines, or other suitable configuration are contemplated. In general, the client and server machines work together to accomplish the processing of the present invention.

Where used, the database(s) is usually connected to the database server component and can be any device which will hold data. For example, the database can be a any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

Computer-Readable Storage Media

The present disclosure also contemplates a computer-readable storage medium (e.g. CD-ROM, memory key, flash memory card, diskette, etc.) having stored thereon a program which, when executed in a computing environment, provides for implementation of algorithms to carry out all or a portion of the results of a response likelihood assessment as described herein. Where the computer-readable medium contains a complete program for carrying out the methods described herein, the program includes program instructions for collecting, analyzing and generating output, and generally includes computer readable code devices for interacting with a user as described herein, processing that data in conjunction with analytical information, and generating unique printed or electronic media for that user.

Where the storage medium provides a program which provides for implementation of a portion of the methods described herein (e.g., the user-side aspect of the methods (e.g., data input, report receipt capabilities, etc.)), the program provides for transmission of data input by the user (e.g., via the internet, via an intranet, etc.) to a computing environment at a remote site. Processing or completion of processing of the data is carried out at the remote site to generate a report. After review of the report, and completion of any needed manual intervention, to provide a complete report, the complete report is then transmitted back to the user as an electronic document or printed document (e.g., fax or mailed paper report). The storage medium containing a program according to the invention can be packaged with instructions (e.g., for program installation, use, etc.) recorded on a suitable substrate or a web address where such instructions may be obtained. The computer-readable storage medium can also be provided in combination with one or more reagents for carrying out response likelihood assessment (e.g., primers, probes, arrays, or other such kit components).

All aspects of the present disclosure may also be practiced such that a limited number of additional genes that are co-expressed with the disclosed genes, for example as evidenced by high Pearson correlation coefficients, are included in a prognostic and/or predictive test in addition to and/or in place of disclosed genes.

Having described exemplary embodiments of the invention, the same will be more readily understood through reference to the following Examples, which are provided by way of illustration, and are not intended to limit the invention in any way. All citations throughout the disclosure are hereby expressly incorporated by reference.

### EXAMPLES

The following examples are offered by way of illustration and not by way of limitation. The disclosures of all citations in the specification are expressly incorporated herein by reference.

### Example 1

### Identification of Differential Markers of Response in Breast Cancer Patients

The data from intergroup trial E2197 (Goldstein L, O'Neill A, Sparano J, et al. E2197: phase III AT (doxorubicin/docetaxel) vs. AC (doxorubicin/cyclophosphamide) in the adjuvant treatment of node positive and high risk node negative breast cancer. Proc Am Soc Clin Oncol. 2005; 23:7s. [Abstract 512]) was used to evaluate the relative efficacy of adjuvant treatment of breast cancer patients with an anthracycline (doxorubicin)+a taxane (AT) compared to an anthracycline (doxorubicin)+cyclophosphamide (AC). The trial compared 4 cycles of a standard doxorubicin-cyclophosphamide (AC) combination given every 3 weeks with 4 cycles of doxorubicin plus docetaxel (AT) in patients with 0-3 positive lymph nodes. The trial was powered to detect a 25% reduction in the disease-free survival (DFS) hazard rate (from an anticipated 5-year DFS of 78% for the AC arm to 83% for the AT arm). Tamoxifen (20 mg daily for 5 years) was recommended for hormone receptor-positive patients following completion of chemotherapy, although approximately 40% of patients eventually took an aromatase inhibitor at some point before or after 5 years. The treatment arms were well balanced with regard to median age (51 years), proportion of lymph node-negative disease (65%), and estrogen receptor (ER)-positive disease (64%).

When single genes by treatment (taxane (T) vs cyclophosphamide (C); or AT vs AC) interactions were evaluated, large numbers of genes with significant interaction effects were observed, in all subjects analyzed; in hormone receptor (HR) positive subjects; in HR positive, Oncotype DX Recurrence Score® value>about 18 subjects; and in HR negative subjects. Most of these interactions are in the same “direction”, i.e., higher expression is associated with greater T benefit and/or less C benefit. Where Oncotype DX Recurrence Score® (RS) was used, the RS was calculated according to the algorithm described in Paik et al., N Engl J Med. 2004 December 30; 351(27):2817-26 and in U.S. application publication No. 20050048542, published Mar. 3, 2005, the entire disclosures of which are expressly incorporated by reference herein.

The predictive utility of PR protein expression was evaluated by immunohistochemistry in a central lab and quantitative RNA expression by RT-PCR for 371 genes (including the 21-gene Recurrence Score [RS]) in a representative sample of 734 patients who received at least 3-4 treatment cycles.

Methods

Patient Selection: All recurrences with available tissue and randomly selected patients without recurrence were identified by an ECOG statistician (ratio 3.5 without recurrence to 1 with recurrence).

Central Immunohistochemistry (IHC) for ER and PR: IHC was performed on two 1.0-mm tissue microarrays (TMAs), using 4 μm sections, DakoCytomation EnVision+ System® (Dako Corporation, Carpinteria, Calif.), and standard methodology using anti-ER antibody (clone 1D5, dilution 1:100) and anti-PR antibody 636 (1:200).

TMAs were reviewed centrally and scored by two pathologists who were blinded to outcomes and local laboratory ER/PR status.

Scoring was performed using the Allred method (see, e.g. Harvey J M, Clark G M, Osborne C K et al. J Clin Oncol 1999; 17:1474-1481) scoring the proportion of positive cells (scored on a 0-5 scale) and staining intensity (scored on a 0-3 scale); proportion and intensity scores were added to yield Allred Score of 0 or 2 through 8 with Allred scores>2 considered positive.

Genes and RT-PCR analysis: Candidate genes were selected to represent multiple biological processes. Quantitative RT-PCR analysis was performed by methods known in the art. For each gene, the appropriate mRNA reference sequence (REFSEQ) accession number was identified and the consensus sequence was accessed through the NCBI Entrez nucleotide database. Appendix 1. Besides the REFSEQ, RT-PCR probe and primer sequences are provided in Appendix 1. Sequences for the amplicons that result from the use of these primer sets are listed in Appendix 2.

Statistical methods: Single Gene by Treatment Interaction Analysis. The objective of this evaluation was to identify genes whose expression, treated as a continuous variable, is differentially associated with the risk of relapse between patients treated with AC versus those treated with AT. A gene expression by treatment interaction model was employed for this purpose and statistical analyses were performed by using Cox Regression models (SAS version 9.1.3). The Cox regression model that was employed for these analyses includes terms for the main effect of treatment, the main effect of gene expression, and the interaction of treatment and gene expression. This model enables prediction of the association between gene expression and the risk of recurrence for patients treated with AC, and of the association between gene expression and the risk of recurrence for patients treated with AT. The point at which these two curves cross is the level of gene expression at which the predicted risk of recurrence is identical if the patient is treated with AC or with AT. This crossover point is easily calculated from the parameter estimates from this model as the negative of the estimated treatment effect, divided by the estimate of the interaction effect.

All hypothesis tests were reported using two-sided p-values, and p-values of <0.05 was considered statistically significant. Relapse-Free Interval was defined as the time from study entry to the first evidence of breast cancer relapse, defined as invasive breast cancer in local, regional or distant sites, including the ipsilateral breast, but excluding new primary breast cancers in the opposite breast. Follow-up for relapse was censored at the time of death without relapse, new primary cancer in the opposite breast, or at the time of the patient was last evaluated for relapse.

The variance of the partial likelihood estimators was estimated with a weighted estimate. See R. Gray, Lifetime Data Anal. 15(1):24-40 (2009); K. Chen K, S-H Lo, Biometrika 86:755-764 (1999).

Individual genes by treatment interactions were tested in Cox models for relapse-free interval (RFI) for the HR+ and HR− patients combined and separately. Since there is little chemotherapy benefit for RS<18, the HR+, RS>18 subset was also analyzed.

The interaction between gene expression and treatment for genes could be depicted graphically. As example we present treatment group-specific plots of the 5-year risk of relapse versus DDR1 gene expression.

Supervised principal components (SPC) was used to combine genes into a multigene predictor of differential treatment benefit, and was evaluated via cross-validation (CV). Pre-validation (PV) inference (Tibshirani and Efron, Stat Appl Genet and Mol Biol 2002; 1:Article1. Epub 2002 Aug. 22), based on 20 replicates of 5 fold cross-validation, was used to estimate and test (via permutations) the utility of the SPC predictors.

Results

Tables 1-4 include an Estimated Coefficient for each response indicator gene listed in the tables in all subjects analyzed (Table 1); in HR+ subjects (Table 2); in HR⁺ subjects having an Oncotype DX Recurrence Score® value greater than about 18 (Table 3); and in HR negative subjects (Table 4). FIGS. 1-4 represent graphically the results for each gene summarized in Tables 1-4, respectively. Each graph of FIGS. 1-4 shows a smooth line representing the model-predicted relationship between expression of the gene and 5-year recurrence rate (RR) in an AC treatment group (the AC prediction curve) and a hatched line representing the model-predicted relationship between gene expression and RR in an AT treatment group (the AT prediction curve). Each of the graphs in FIGS. 1-4 are presented with 5-year risk of recurrence on the y-axis and normalized expression (Cₜ) on the x-axis, where increasing normalized Cₜ values indicate increasing expression levels.

The Estimated Coefficient referred to in Tables 1-4 is a reflection of the difference between the slopes in the Cox regression model of the AC prediction curve and the AT prediction curve. The magnitude of the Estimated Coefficient is related to the difference between the slopes of the AC prediction curve and the AT prediction curve; the sign of the Estimated Coefficient is an indication of which treatment (AT or AC) becomes the favored treatment as expression of the gene increases. For example, in Table 1, the Estimated Coefficient for SLC1A3 is −0.7577. The magnitude (absolute value=0.7577) is related to the difference between the slopes of the AC prediction curve and the AT prediction curve (shown in the first panel of FIG. 1) for SLC1A3 in this population (all patients, i.e. not stratified by hormone receptor status or by RS). The negative sign indicates that higher expression levels of SLC1A3 favor treatment with AT while lower expression levels of SLC1A3 favor treatment with AC.

The p-value given in Table 1 is a measure of the statistical significance of the difference between the slope of the AC prediction curve and the slope of the AT prediction curve in the Cox regression model, i.e. the probability that the observed difference in slopes is due to chance. Smaller p-values indicate greater statistical significance.

Analysis of Gene Expression in all Patients in Study Population (Irrespective of HR Status and Oncotype Dx® RS Score)

Table 1 shows a list of 76 genes whose normalized expression level is differentially associated with response to AT vs. AC treatment in all patients. When the estimated coefficient is <0, high expression of that gene is indicative that AT treatment is more effective than AC treatment; low gene expression of that gene is indicative that AC treatment is more effective than AT treatment. When the estimated coefficient is >0, high expression of that gene is indicative that AC treatment is more effective than AT treatment; low expression of that gene is indicative that AT treatment is more effective than AC treatment.

As noted above, FIG. 1 shows a graph for each gene in Table 1. Each graph shows a smooth line representing the model-predicted relationship between expression of the gene and 5-year recurrence rate (RR) in an AC treatment group (the AC prediction curve) and a hatched line representing the model-predicted relationship between gene expression and RR in an AT treatment group (the AT prediction curve). For each gene, the AC prediction curve and the AT prediction curve have statistically significant different slopes in the Cox regression model, indicating that AC or AT can be chosen as a favored treatment based, at least in part, on the expression of the gene. The graph for each gene also shows, as a horizontal dashed line, represents the 12.3% recurrence rate at 5-year RR in all patients analyzed (i.e., without regard to HR status or Oncotype Dx RS).

The first panel of FIG. 1, for example, shows the AC-prediction curve and the AT prediction curve for SLC1A3. The curves have significantly different slopes in the Cox regression model and the lines cross, resulting in the ability to discriminate, based on the expression level of SLC1A3, patients who are more likely to respond to AT (or to AC). For SLC1A3, patients with higher expression levels are more likely to respond to AT than AC, while patients with lower expression levels are more likely to respond to AC than AT.

Analysis of Gene Expression in HR+ Patients in Study Population

Table 2 shows a list of 97 genes having a normalized expression level that is differently correlated with response to AT vs. AC in hormone receptor (HR)-positive patients (without regard to Oncotype Dx RS value). When the estimated coefficient is <0, high expression of that gene is indicative that AT treatment is more effective than AC treatment; low expression of that gene is indicative that AC treatment is more effective than AT treatment. When the estimated coefficient is >0, high expression of that gene is indicative than AC treatment is more effective than AT treatment; low expression of that gene is indicative that AT treatment is more effective than AC treatment.

The data summarized in Table 2 are provided in graph form for each gene in FIG. 2. For each gene, the AC prediction curve and the AT prediction curve have statistically significant different slopes in the Cox regression model, indicating that AC or AT can be chosen as a favored treatment based, at least in part, on the expression of the gene. The graph for each gene also shows, as a horizontal dashed line represents the 10.0% recurrence rate at 5-year RR in HR-positive patients.

Analysis of Gene Expression in HR+ Patients in the Study Population Having an Oncotype Dx RS of about 18 or Greater

Table 3 shows a list of 165 genes whose normalized expression level is differentially associated with response to AT vs. AC in HR-positive patients having a Recurrence Score (RS)>18. These patients have an increased likelihood of cancer recurrence. When the estimated coefficient is <0, high expression of that gene is indicative that AT treatment is more effective than AC treatment; low expression of that gene is indicative that AC treatment is more effective than AT treatment. When the estimated coefficient is >0, high expression of that gene is indicative that AC treatment is more effective than AT treatment; low expression of that gene is indicative that AT treatment is more effective than AC treatment.

The data summarized in Table 3 are provided in graph form for each gene in FIG. 3. For each gene, the AC prediction curve and the AT prediction curve have statistically significant different slopes in the Cox regression model, indicating that AC or AT can be chosen as a favored treatment based, at least in part, on the expression of the gene. The graph for each gene also shows, as a horizontal dashed line represents the 14.9% recurrence rate at 5-year RR in the HR-positive patient group having an Oncotype Dx RS of about 18 or greater.

Analysis of Gene Expression in HR− Patients in Study Population

Table 4 shows a list of 9 genes whose normalized expression level is differentially associated with response to AT vs. AC treatment in HR-negative patients.

The data summarized in Table 4 is provided in graph form for each gene in FIG. 4. For each gene, the AC prediction curve and the AT prediction curve have statistically significant different slopes in the Cox regression model, indicating that AC or AT can be chosen as a favored treatment based, at least in part, on the expression of the gene. The graph for each gene also shows, as a horizontal dashed line represents the 16.9% recurrence rate at 5-year RR in the HR-negative patient group.

Discussion

PR Analysis. There was a weak benefit for AT in PR-negative (AT vs AC hazard ratio [RR]=0.75; p=0.06) and AC in PR-positive disease (RR=1.37; p=0.05) by central immunhistochemistry (Allred score>2 positive) but not when genomic PR was evaluated by RT-PCR (>5.5 units positive).

RS and Genes Analyzed. Table 1 illustrates genes that can be used as markers of benefit of taxane therapy irrespective of hormone receptor expression status, and facilitate selection of AC vs AT therapy. (Table 1). Several genes strongly predicted taxane benefit when assessed in the context of AT vs AC therapy in the HR-positive subset (Table 2), and especially in the HR-positive, Oncotype Dx RS>18 subset (Table 3).

Nine genes were identified for which gene expression can be used as markers of benefit of taxane therapy in hormone receptor (HR)-negative breast cancer, and could be used to assess AT vs. AC benefit in the hormone receptor (HR)-negative patients (Table 4).

Of the genes listed in Table 1, SLC1A3 (glial high affinity glutamase transporter 3) is a member of a large family of solute transport proteins, located within the multiple sclerosis locus on 5p.

Of the genes identified in the HR-positive subset (Table 2), DDR1 (discoidin domain receptor 1) is a transmembrane receptor TK the aberrant expression and signaling of which has been linked to accelerated matrix degradation and remodeling, including tumor invasion. Collagen-induced DDR1 activation is believed to be involved in normal mammary cell adhesion, and may distinguish between invasive ductal carcinoma (IDC) and invasive lobular carcinoma (ILC), and further may induce cyclooxygenase-2 and promoter chemoresistance through the NF-κB pathway. EIF4E2 (human transcription initiation factor 4) is an mRNA cap-binding protein.

When differential response markers in HR-positive, RS>18 patients (Table 3) are ranked in ascending order by p-value, DDR1, RELA, ZW10, and RhoB are four of the top five genes. RELA is an NF-κB subunit, which plays a role in inflammation, innate immunity, cancer and anti-apoptosis. This gene has also been associated with chemoresistance, and may be necessary for IL-6 induction, which is involved in immune cell homeostasis. ZW10 is a kinetochore protein involved in mitotic spindle formation. It is part of the ROD-ZW10-Zwilch complex, and binds tubulin. RhoB is a low molecular weight GPTase belonging to the RAS superfamily. The Rho protein is pivotal in regulation of actin cytoskeleton. RhoB acts as tumor suppressor gene and inhibits tumor growth and metastases in vitro and in vivo, and activates NF-κB. KO mice for RhoB show increased sensitivity to chemical carcinogenesis and resistance to radiation and cytotoxic induced apoptosis.

DDR1, RELA and RhoB are key elements in the NFκB signaling pathway. Based on these findings, it is expected that other genes in the NFκB pathway are likely to be differentially associated with response to AT vs. AC treatment in HR-positive patients at high risk for cancer recurrence, and such can be used as differential response markers for AT vs. AC treatment. Some additional genes that are known to be involved in NFκB signaling are shown in Table 5.

In the HR-negative subset, CD247 exhibited a correlation of expression with AT vs. AC therapy (p-value<0.01) and exhibited a strong correlation indicating that expression was positively correlated with increased likelihood of benefit of treatment including a taxane (FIG. 4). The estimated coefficient<0 indicates that high gene expression favors AT treatment, while low gene expression favors AC treatment (see also FIG. 4). CD247, also known as T cell receptor zeta (TCRzeta) functions as an amplification module of the TCR signaling cascade. This gene is downregulated in many chronic infectious and inflammatory processes, such as systemic lupus erythematosus (SLE).

FIG. 5 illustrates an exemplary treatment group-specific plot of the 5-year risk of relapse versus gene expression presented for an exemplary gene, DDR1.

### Example 2

### ESR1 Gene Combinations

Using the differential response markers identified in Table 2, supervised principle component analysis was carried out in HR+RS>18 patients treated with AT vs AC according the methods of Bair E, Hastie T, Paul D, Tibshirani R. Prediction by supervised principal components. J. Amer. Stat. Assoc. 101:119-137, 2006.

Principal Components can be used in regression problems for dimensionality reduction in a data set by keeping the most important principal components and ignoring the other ones. Supervised principal components (Bair et al. supra) is similar to conventional principal components analysis except that it uses a subset of the predictors (i.e. individual genes) that are selected based on their association with relapse-free interval (assessed using Cox regression). In the present example, only the first component was utilized to obtain a score from a weighted combination of genes.

In this patient group, the most heavily weighted gene by supervised principle components analysis was ESR1, indicating that ESR1 is particularly useful when used in combinations with any of the other genes listed in Table 3 in predicting differential response to taxane vs. cyclophosphamide in HR+high recurrence risk patients. Exemplary combinations of genes include, without limitation:

DDR1+ESR1, ZW10+ESR1, RELA+ESR1, BAX+ESR1, RHOB+ESR1, TSPAN4+ESR1, BBC3+ESR1, SHC1+ESR1, CAPZA1+ESR1, STK10+ESR1, TBCC+ESR1, EIF4E2+ESR1, MCL1+ESR1, RASSF1+ESR1, VEGF+ESR1, SLC1A3+ESR1, DICER1+ESR1, ILK+ESR1, FAS+ESR1, RAB6C+ESR1, ESR1+ESR1, MRE11A+ESR1, APOE+ESR1, BAK1+ESR1, UFM1+ESR1, AKT2+ESR1, SIRT1+ESR1, BCL2L13+ESR1, ACTR2+ESR1, LIMK2+ESR1, HDAC6+ESR1, RPN2+ESR1, PLD3+ESR1, CHGA+ESR1, RHOA+ESR1, MAPK14+ESR1, ECGF1+ESR1, MAPRE1+ESR1, HSPA1B+ESR1, GATA3+ESR1, PPP2CA+ESR1, ABCD1+ESR1, MAD2L1BP+ESR1, VHL+ESR1, GCLC+ESR1, ACTB+ESR1, BCL2L11+ESR1, PRDX1+ESR1, LILRB1+ESR1, GNS+ESR1, CHFR+ESR1, CD68+ESR1, LIMK1+ESR1, GADD45B+ESR1, VEGFB+ESR1, APRT+ESR1, MAP2K3+ESR1, MGC52057+ESR1, MAPK3+ESR1, APC+ESR1, RAD1+ESR1, COL6A3+ESR1, RXRB+ESR1, CCT3+ESR1, ABCC3+ESR1, GPX1+ESR1, TUBB2C+ESR1, HSPA1A+ESR1, AKT1+ESR1, TUBA6+ESR1, TOP3B+ESR1, CSNK1D+ESR1, SOD1+ESR1, BUB3+ESR1, MAP4+ESR1, NFKB1+ESR1, SEC61A1+ESR1, MAD1L1+ESR1, PRKCH+ESR1, RXRA+ESR1, PLAU+ESR1, CD63+ESR1, CD14+ESR1, RHOC+ESR1, STAT1+ESR1, NPC2+ESR1, NME6+ESR1, PDGFRB+ESR1, MGMT+ESR1, GBP1+ESR1, ERCC1+ESR1, RCC1+ESR1, FUS+ESR1, TUBA3+ESR1, CHEK2+ESR1, APOC1+ESR1, ABCC10+ESR1, SRC+ESR1, TUBB+ESR1, FLAD1+ESR1, MAD2L2+ESR1, LAPTM4B+ESR1, REG1A+ESR1, PRKCD+ESR1, CST7+ESR1, IGFBP2+ESR1, FYN+ESR1, KDR+ESR1, STMN1+ESR1, ZWILCH+ESR1, RBM17+ESR1, TP53BP1+ESR1, CD247+ESR1, ABCA9+ESR1, NTSR2+ESR1, FOS+ESR1, TNFRSF10A+ESR1, MSH3+ESR1, PTEN+ESR1, GBP2+ESR1, STK11+ESR1, ERBB4+ESR1, TFF1+ESR1, ABCC1+ESR1, IL7+ESR1, CDC25B+ESR1, TUBD1+ESR1, BIRC4+ESR1, ACTR3+ESR1, SLC35B1+ESR1, COL1A1+ESR1, FOXA1+ESR1, DUSP1+ESR1, CXCR4+ESR1, IL2RA+ESR1, GGPS1+ESR1, KNS2+ESR1, RB1+ESR1, BCL2L1+ESR1, XIST+ESR1, BIRC3+ESR1, BID+ESR1, BCL2+ESR1, STAT3+ESR1, PECAM1+ESR1, DIABLO+ESR1, CYBA+ESR1, TBCE+ESR1, CYP1B1+ESR1, APEX1+ESR1, TBCD+ESR1, HRAS+ESR1, TNFRSF10B+ESR1, ELP3+ESR1, PIK3C2A+ESR1, HSPA5+ESR1, VEGFC+ESR1, CRABP1+ESR1, MMP11+ESR1, SGK+ESR1, CTSD+ESR1, BAD+ESR1, PTPN21+ESR1, HSPA9B+ESR1, and PMS1+ESR1

Any combination of two or more genes from Table 3, said combination not comprising ESR1 is also expected to be useful in predicting differential response to taxane vs. cyclophosphamide in HR+high recurrence risk patients.

Similarly it is expected that ESR1 is particularly useful when used in combinations with any of the other genes listed in Table 2 in predicting differential response to taxane vs. cyclophosphamide in HR+ patients. Exemplary combinations of genes include:

DDR1+ESR1, EIF4E2+ESR1, TBCC+ESR1, STK10+ESR1, ZW10+ESR1, BBC3+ESR1, BAX+ESR1, BAK1+ESR1, TSPAN4+ESR1, SLC1A3+ESR1, SHC1+ESR1, CHFR+ESR1, RHOB+ESR1, TUBA6+ESR1, BCL2L13+ESR1, MAPRE1+ESR1, GADD45B+ESR1, HSPA1B+ESR1, FAS+ESR1, TUBB+ESR1, HSPA1A+ESR1, MCL1+ESR1, CCT3+ESR1, VEGF+ESR1, TUBB2C+ESR1, AKT1+ESR1, MAD2L1BP+ESR1, RPN2+ESR1, RHOA+ESR1, MAP2K3+ESR1, BID+ESR1, APOE+ESR1, ESR1+ESR1, ILK+ESR1, NTSR2+ESR1, TOP3B+ESR1, PLD3+ESR1, DICER1+ESR1, VHL+ESR1, GCLC+ESR1, RAD1+ESR1, GATA3+ESR1, CXCR4+ESR1, NME6+ESR1, UFM1+ESR1, BUB3+ESR1, CD14+ESR1, MRE11A+ESR1, CST7+ESR1, APOC1+ESR1, GNS+ESR1, ABCC5+ESR1, AKT2+ESR1, APRT+ESR1, PLAU+ESR1, RCC1+ESR1, CAPZA1+ESR1, RELA+ESR1, NFKB1+ESR1, RASSF1+ESR1, BCL2L11+ESR1, CSNK1D+ESR1, SRC+ESR1, LIMK2+ESR1, SIRT1+ESR1, RXRA+ESR1, ABCD1+ESR1, MAPK3+ESR1, CDCA8+ESR1, DUSP1+ESR1, ABCC1+ESR1, PRKCH+ESR1, PRDX1+ESR1, TUBA3+ESR1, VEGFB+ESR1, LILRB1+ESR1, LAPTM4B+ESR1, HSPA9B+ESR1, ECGF1+ESR1, GDF15+ESR1, ACTR2+ESR1, IL7+ESR1, HDAC6+ESR1, ZWILCH+ESR1, CHEK2+ESR1, REG1A+ESR1, APC+ESR1, SLC35B1+ESR1, NEK2+ESR1, ACTB+ESR1, BUB1+ESR1, PPP2CA+ESR1, TNFRSF10A+ESR1, TBCD+ESR1, ERBB4+ESR1, CDC25B+ESR1, and STMN1+ESR1.

A combination of two or more genes from Table 2, said combination not comprising ESR1 is also expected to be useful in predicting differential response to taxane vs. cyclophosphamide in HR+ patients at high recurrence risk for cancer.

### Example 3

### Genes of the NFκB Pathway

When the differential response markers in HR-positive, RS>18 patients are ranked in ascending order of p-value, three of the top five revealed genes are DDR1, RELA and RHOB. The RELA gene encodes one of the principle subunits of the NFκB transcription factor. Therefore, it is notable that both the DDR1 gene and the RHOB gene stimulate the NFκB signaling pathway. These results indicate that additional genes that stimulate the activity of the NFκB pathway, given in Table 5, also predict increased likelihood of response to AT vs. AC chemotherapy.

### Example 4

### Gene Expression Profiling Protocol

Breast tumor formalin-fixed and paraffin-embedded (FPE) blocks or frozen tumor sections are provided. Fixed tissues are incubated for 5 to 10 hours in 10% neutral-buffered formalin before being alcohol-dehydrated and embedded in paraffin.

RNA is extracted from three 10-μm FPE sections per each patient case. Paraffin is removed by xylene extraction followed by ethanol wash. RNA is isolated from sectioned tissue blocks using the MasterPure Purification kit (Epicenter, Madison, Wis.); a DNase I treatment step is included. RNA is extracted from frozen samples using Trizol reagent according to the supplier's instructions (Invitrogen Life Technologies, Carlsbad, Calif.). Residual genomic DNA contamination is assayed by a TaqMan® (Applied Biosystems, Foster City, Calif.) quantitative PCR assay (no RT control) for β-actin DNA. Samples with measurable residual genomic DNA are resubjected to DNase I treatment, and assayed again for DNA contamination. TaqMan is a registered trademark of Roche Molecular Systems.

RNA is quantitated using the RiboGreen® fluorescence method (Molecular Probes, Eugene, Oreg.), and RNA size is analyzed by microcapillary electrophoresis using an Agilent 2100 Bioanalyzer (Agilent Technologies, Palo Alto, Calif.).

Reverse transcription (RT) is performed using a SuperScript® First-Strand Synthesis kit for RT-PCR (Invitrogen Corp., Carlsbad, Calif.). Total FPE RNA and pooled gene-specific primers are present at 10 to 50 ng/μl and 100 nmol/L (each), respectively.

TaqMan reactions are performed in 384-well plates according to instructions of the manufacturer, using Applied Biosystems Prism 7900HT TaqMan instruments. Expression of each gene is measured either in duplicate 5-μl reactions using cDNA synthesized from 1 ng of total RNA per reaction well, or in single reactions using cDNA synthesized from 2 ng of total RNA. Final primer and probe concentrations are 0.9 μmol/L (each primer) and 0.2 μmol/L, respectively. PCR cycling is performed as follows: 95° C. for 10 minutes for one cycle, 95° C. for 20 seconds, and 60° C. for 45 seconds for 40 cycles. To verify that the RT-PCR signals derives from RNA rather than genomic DNA, for each gene tested a control identical to the test assay but omitting the RT reaction (no RT control) is included. The threshold cycle for a given amplification curve during RT-PCR occurs at the point the fluorescent signal from probe cleavage grows beyond a specified fluorescence threshold setting. Test samples with greater initial template exceed the threshold value at earlier amplification cycle numbers than those with lower initial template quantities.

For normalization of extraneous effects, cycle threshold (CT) measurements obtained by RT-PCR were normalized relative to the mean expression of a set of five reference genes: ATP5E, PGK1, UBB, VDAC2, and GPX1. A one unit increase in reference normalized expression measurements generally reflects a 2-fold increase in RNA quantity.

While the present invention has been described with reference to what are considered to be the specific embodiments, it is to be understood that the invention is not limited to such embodiments. To the contrary, the invention is intended to cover various modifications and equivalents included within the spirit and scope of the appended claims.

APPENDIX 1

| | | | | |
|---|---|---|---|---|
| Gene Name | Accession # | Oligo Name | Oligo Sequence | SEQ ID NO |

| | | | | |
|---|---|---|---|---|
| ABCA9 | NM_172386 | T2132/ABCA9.f1 | TTACCCGTGGGAACTGTCTC | 1 |
| ABCA9 | NM_172386 | T2133/ABCA9.r1 | GACCAGTAAATGGGTCAGAGGA | 2 |
| ABCA9 | NM_172386 | T2134/ABCA9.p1 | TCCTCTCACCAGGACAACAACCACA | 3 |
| ABCB1 | NM_000927 | S8730/ABCB1.f5 | AAACACCACTGGAGCATTGA | 4 |
| ABCB1 | NM_000927 | S8731/ABCB1.r5 | CAAGCCTGGAACCTATAGCC | 5 |
| ABCB1 | NM_000927 | S8732/ABCB1.p5 | CTCGCCAATGATGCTGCTCAAGTT | 6 |
| ABCB5 | NM_178559 | T2072/ABCB5.f1 | AGACAGTCGCCTTGGTCG | 7 |
| ABCB5 | NM_178559 | T2073/ABCB5.r1 | AACCTCTGCAGAAGCTGGAC | 8 |
| ABCB5 | NM_178559 | T2074/ABCB5.p1 | CCGTACTCTTCCCACTGCCATTGA | 9 |
| ABCC10 | NM_033450 | S9064/ABCC10.f1 | ACCAGTGCCACAATGCAG | 10 |
| ABCC10 | NM_033450 | S9065/ABCC10.r1 | ATAGCGCTGACCACTGCC | 11 |
| ABCC10 | NM_033450 | S9066/ABCC10.p1 | CCATGAGCTGTAGCCGAATGTCCA | 12 |
| ABCC11 | NM_032583 | T2066/ABCC11.f1 | AAGCCACAGCCTCCATTG | 13 |
| ABCC11 | NM_032583 | T2067/ABCC11.r1 | GGAAGGCTTCACGGATTGT | 14 |
| ABCC11 | NM_032583 | T2068/ABCC11.p1 | TGGAGACAGACACCCTGATCCAGC | 15 |
| ABCC5 | NM_005688 | S5605/ABCC5.f1 | TGCAGACTGTACCATGCTGA | 16 |
| ABCC5 | NM_005688 | S5606/ABCC5.r1 | GGCCAGCACCATAATCCTAT | 17 |
| ABCC5 | NM_005688 | S5607/ABCC5.p1 | CTGCACACGGTTCTAGGCTCCG | 18 |
| ABCD1 | NM_000033 | T1991/ABCD1.f1 | TCTGTGGCCCACCTCTACTC | 19 |
| ABCD1 | NM_000033 | T1992/ABCD1.r1 | GGGTGTAGGAAGTCACAGCC | 20 |
| ABCD1 | NM_000033 | T1993/ABCD1.p1 | AACCTGACCAAGCCACTCCTGGAC | 21 |
| ACTG2 | NM_001615 | S4543/ACTG2.f3 | ATGTACGTCGCCATTCAAGCT | 22 |
| ACTG2 | NM_001615 | S4544/ACTG2.r3 | ACGCCATCACCTGAATCCA | 23 |
| ACTG2 | NM_001615 | S4545/ACTG2.p3 | CTGGCCGCACGACAGGCATC | 24 |
| ACTR2 | NM_005722 | T2380/ACTR2.f1 | ATCCGCATTGAAGACCCA | 25 |
| ACTR2 | NM_005722 | T2381/ACTR2.r1 | ATCCGCTAGAACTGCACCAC | 26 |
| ACTR2 | NM_005722 | T2382/ACTR2.p1 | CCCGCAGAAAGCACATGGTATTCC | 27 |
| ACTR3 | NM_005721 | T2383/ACTR3.f1 | CAACTGCTGAGAGACCGAGA | 28 |
| ACTR3 | NM_005721 | T2384/ACTR3.r1 | CGCTCCTTTACTGCCTTAGC | 29 |
| ACTR3 | NM_005721 | T2385/ACTR3.p1 | AGGAATCCCTCCAGAACAATCCTTGG | 30 |
| AK055699 | NM_194317 | S2097/AK0556.f1 | CTGCATGTGATTGAATAAGAAACAAGA | 31 |
| AK055699 | NM_194317 | S2098/AK0556.r1 | TGTGGACCTGATCCCTGTACAC | 32 |
| AK055699 | NM_194317 | S5057/AK0556.p1 | TGACCACACCAAAGCCTCCCTGG | 33 |
| AKT1 | NM_005163 | S0010/AKT1.f3 | CGCTTCTATGGCGCTGAGAT | 34 |
| AKT1 | NM_005163 | S0012/AKT1.r3 | TCCCGGTACACCACGTTCTT | 35 |
| AKT1 | NM_005163 | S4776/AKT1.p3 | CAGCCCTGGACTACCTGCACTCGG | 36 |
| AKT2 | NM_001626 | S0828/AKT2.f3 | TCCTGCCACCCTTCAAACC | 37 |
| AKT2 | NM_001626 | S0829/AKT2.r3 | GGCGGTAAATTCATCATCGAA | 38 |
| AKT2 | NM_001626 | S4727/AKT2.p3 | CAGGTCACGTCCGAGGTCGACACA | 39 |
| AKT3 | NM_005465 | S0013/AKT3.f2 | TTGTCTCTGCCTTGGACTATCTACA | 40 |
| AKT3 | NM_005465 | S0015/AKT3.r2 | CCAGCATTAGATTCTCCAACTTGA | 41 |
| AKT3 | NM_005465 | S4884/AKT3.p2 | TCACGGTACACAATCTTTCCGGA | 42 |
| ANXA4 | NM_001153 | T1017/ANXA4.f1 | TGGGAGGGATGAAGGAAAT | 43 |
| ANAX4 | NM_001153 | T1018/ANXA4.r1 | CTCATACAGGTCCTGGGCA | 44 |
| ANXA4 | NM_001153 | T1019/ANXA4.p1 | TGTCTCACGAGAGCATCGTCCAGA | 45 |
| APC | NM_000038 | S0022/APC.f4 | GGACAGCAGGAATGTGTTTC | 46 |
| APC | NM_000038 | S0024/APC.r4 | ACCCACTCGATTTGTTTCTG | 47 |
| APC | NM_000038 | S4888/APC.p4 | CATTGGCTCCCCGTGACCTGTA | 48 |
| APEX-1 | NM_001641 | S9947/APEX-1.f1 | GATGAAGCCTTTCGCAAGTT | 49 |
| APEX-1 | NM_001641 | S9948/APEX-1.r1 | AGGTCTCCACACAGCACAAG | 50 |
| APEX-1 | NM_001641 | S9949/APEX-1.p1 | CTTTCGGGAAGCCAGGCCCTT | 51 |
| APOC1 | NM_001645 | S9667/APOC1.f2 | GGAAACACACTGGAGGACAAG | 52 |
| APOC1 | NM_001645 | S9668/APOC1.r2 | CGCATCTTGGCAGAAAGTT | 53 |
| APOC1 | NM_001645 | S9669/APOC1.p2 | TCATCAGCCGCATCAAACAGAGTG | 54 |
| APOD | NM_001647 | T0536/APOD.f1 | GTTTATGCCATCGGCACC | 55 |
| APOD | NM_001647 | T0537/APOD.r1 | GGAATACACGAGGGCATAGTTC | 56 |
| APOD | NM_001647 | T0538/APOD.p1 | ACTGGATCCTGGCCACCGACTATG | 57 |
| APOE | NM_000041 | T1994/APOE.f1 | GCCTCAAGAGCTGGTTCG | 58 |
| APOE | NM_000041 | T1995/APOE.r1 | CCTGCACCTTCTCCACCA | 59 |
| APOE | NM_000041 | T1996/APOE.p1 | ACTGGCGCTGCATGTCTTCCAC | 60 |
| APRT | NM_000485 | T1023/APRT.f1 | GAGGTCCTGGAGTGCGTG | 61 |
| APRT | NM_000485 | T1024/APRT.r1 | AGGTGCCAGCTTCTCCCT | 62 |
| APRT | NM_000485 | T1025/APRT.p1 | CCTTAAGCGAGGTCAGCTCCACCA | 63 |
| ARHA | NM_001664 | S8372/ARHA.f1 | GGTCCTCCGTCGGTTCTC | 64 |
| ARHA | NM_001664 | S8373/ARHA.r1 | GTCGCAAACTCGGAGACG | 65 |
| ARHA | NM_001664 | S8374/ARHA.p1 | CCACGGTCTGGTCTTCAGCTACCC | 66 |
| AURKB | NM_004217 | S7250/AURKB.f1 | AGCTGCAGAAGAGCTGCACAT | 67 |
| AURKB | NM_004217 | S7251/AURKB.r1 | GCATCTGCCAACTCCTCCAT | 68 |
| AURKB | NM_004217 | S7252/AURKB.p1 | TGACGAGCAGCGAACAGCCACG | 69 |
| B-actin | NM_001101 | S0034/B-acti.f2 | CAGCAGATGTGGATCAGCAAG | 70 |
| B-actin | NM_001101 | S0036/B-acti.r2 | GCATTTGCGGTGGACGAT | 71 |
| B-actin | NM_001101 | S4730/B-acti.p2 | AGGAGTATGACGAGTCCGGCCCC | 72 |
| B-Catenin | NM_001904 | S2150/B-Cate.f3 | GGCTCTTGTGCGTACTGTCCTT | 73 |
| B-Catenin | NM_001904 | S2151/B-Cate.r3 | TCAGATGACGAAGAGCACAGATG | 74 |
| B-Catenin | NM_001904 | S5046/B-Cate.p3 | AGGCTCAGTGATGTCTTCCCTGTCACCAG | 75 |
| BAD | NM_032989 | S2011/BAD.f1 | GGGTCAGGTGCCTCGAGAT | 76 |
| BAD | NM_032989 | S2012/BAD.r1 | CTGCTCACTCGGCTCAAACTC | 77 |
| BAD | NM_032989 | S5058/BAD.p1 | TGGGCCCAGAGCATGTTCCAGATC | 78 |
| BAG1 | NM_004323 | S1386/BAG1.f2 | CGTTGTCAGCACTTGGAATACAA | 79 |
| BAG1 | NM_004323 | S1387/BAG1.r2 | GTTCAACCTCTTCCTGTGGACTGT | 80 |
| BAG1 | NM_004323 | S4731/BAG1.p2 | CCCAATTAACATGACCCGGCAACCAT | 81 |
| Bak | NM_001188 | S0037/Bak.f2 | CCATTCCCACCATTCTACCT | 82 |
| Bak | NM_001188 | S0039/Bak.r2 | GGGAACATAGACCCACCAAT | 83 |
| Bak | NM_001188 | S4724/Bak.p2 | ACACCCCAGACGTCCTGGCCT | 84 |
| Bax | NM_004324 | S0040/Bax.f1 | CCGCCGTGGACACAGACT | 85 |
| Bax | NM_004324 | S0042/Bax.r1 | TTGCCGTCAGAAAACATGTCA | 86 |
| Bax | NM_004324 | S4897/Bax.p1 | TGCCACTCGGAAAAAGACCTCTCGG | 87 |
| BBC3 | NM_014417 | S1584/BBC3.f2 | CCTGGAGGGTCCTGTACAAT | 88 |
| BBC3 | NM_014417 | S1585/BBC3.r2 | CTAATTGGGCTCCATCTCG | 89 |
| BBC3 | NM_014417 | S4890/BBC3.p2 | CATCATGGGACTCCTGCCCTTACC | 90 |
| Bcl2 | NM_000633 | S0043/Bcl2.f2 | CAGATGGACCTAGTACCCACTGAGA | 91 |
| Bcl2 | NM_000633 | S0045/Bcl2.r2 | CCTATGATTTAAGGGCATTTTTCC | 92 |
| Bcl2 | NM_000633 | S4732/Bcl2.p2 | TTCCACGCCGAAGGACAGCGAT | 93 |
| BCL2L11 | NM_138621 | S7139/BCL2L1.f1 | AATTACCAAGCAGCCGAAGA | 94 |
| BCL2L11 | NM_138621 | S7140/BCL2L1.r1 | CAGGCGGACAATGTAACGTA | 95 |
| BCL2L11 | NM_138621 | S7141/BCL2L1.p1 | CCACCCACGAATGGTTATCTTACGACTG | 96 |
| BCL2L13 | NM_015367 | S9025/BCL2L1.f1 | CAGCGACAACTCTGGACAAG | 97 |
| BCL2L13 | NM_015367 | S9026/BCL2L1.r1 | GCTCTCAGACTGCCAGGAA | 98 |
| BCL2L13 | NM_015367 | S9027/BCL2L1.p1 | CCCCAGAGTCTCCAACTGTGACCA | 99 |
| Bclx | NM_001191 | S0046/Bclx.f2 | CTTTTGTGGAACTCTATGGGAACA | 100 |
| Bclx | NM_001191 | S0048/Bclx.r2 | CAGCGGTTGAAGCGTTCCT | 101 |
| Bclx | NM_001191 | S4898/Bclx.p2 | TTCGGCTCTCGGCTGCTGCA | 102 |
| BCRP | NM_004827 | S0840/BCRP.f1 | TGTACTGGCGAAGAATATTTGGTAAA | 103 |
| BCRP | NM_004827 | S0841/BCRP.r1 | GCCACGTGATTCTTCCACAA | 104 |
| BCRP | NM_004827 | S4836/BCRP.p1 | CAGGGCATCGATCTCTCACCCTGG | 105 |
| BID | NM_001196 | S6273/BID.f3 | GGACTGTGAGGTCAACAACG | 106 |
| BID | NM_001196 | S6274/BID.r3 | GGAAGCCAAACACCAGTAGG | 107 |
| BID | NM_001196 | S6275/BID.p3 | TGTGATGCACTCATCCCTGAGGCT | 108 |
| BIN1 | NM_004305 | S2651/BIN1.f3 | CCTGCAAAAGGGAACAAGAG | 109 |
| BIN1 | NM_004305 | S2652/BIN1.r3 | CGTGGTTGACTCTGATCTCG | 110 |
| BIN1 | NM_004305 | S4954/BIN1.p3 | CTTCGCCTCCAGATGGCTCCC | 111 |
| BRCA1 | NM_007295 | S0049/BRCA1.f2 | TCAGGGGGCTAGAAATCTGT | 112 |
| BRCA1 | NM_007295 | S0051/BRCA1.r2 | CCATTCCAGTTGATCTGTGG | 113 |
| BRCA1 | NM_007295 | S4905/BRCA1.p2 | CTATGGGCCCTTCACCAACATGC | 114 |
| BRCA2 | NM_000059 | S0052/BRCA2.f2 | AGTTCGTGCTTTGCAAGATG | 115 |
| BRCA2 | NM_000059 | S0054/BRCA2.r2 | AAGGTAAGCTGGGTCTGCTG | 116 |
| BRCA2 | NM_000059 | S4985/BRCA2.p2 | CATTCTTCACTGCTTCATAAAGCTCTGCA | 117 |
| BUB1 | NM_004336 | S4294/BUB1.f1 | CCGAGGTTAATCCAGCACGTA | 118 |
| BUB1 | NM_004336 | S4295/BUB1.r1 | AAGACATGGCGCTCTCAGTTC | 119 |
| BUB1 | NM_004336 | S4296/BUB1.p1 | TGCTGGGAGCCTACACTTGGCCC | 120 |
| BUB1B | NM_001211 | S8060/BUB1B.f1 | TCAACAGAAGGCTGAACCACTAGA | 121 |
| BUB1B | NM_001211 | S8061/BUB1B.r1 | CAACAGAGTTTGCCGAGACACT | 122 |
| BUB1B | NM_001211 | S8062/BUB1B.p1 | TACAGTCCCAGCACCGACAATTCC | 123 |
| BUB3 | NM_004725 | S8475/BUB3.f1 | CTGAAGCAGATGGTTCATCATT | 124 |
| BUB3 | NM_004725 | S8476/BUB3.r1 | GCTGATTCCCAAGAGTCTAACC | 125 |
| BUB3 | NM_004725 | S8477/BUB3.p1 | CCTCGCTTTGTTTAACAGCCCAGG | 126 |
| c-Src | NM_005417 | S7320/c-Src.f1 | TGAGGAGTGGTATTTTGGCAAGA | 127 |
| c-Src | NM_005417 | S7321/c-Src.r1 | CTCTCGGGTTCTCTGCATTGA | 128 |
| c-Src | NM_005417 | S7322/c-Src-p1 | AACCGCTCTGACTCCCGTCTGGTG | 129 |
| C14orf10 | NM_017917 | T2054/C14orf.f1 | GTCAGCGTGGTAGCGGTATT | 130 |
| C14orf10 | NM_017917 | T2055/C14orf.r1 | GGAAGTCTTGGCTAAAGAGGC | 131 |
| C14orf10 | NM_017917 | T2056/C14orf.p1 | AACAATTACTGTCACTGCCGCGGA | 132 |
| C20 orf1 | NM_012112 | S3560/C20 or.f1 | TCAGCTGTGAGCTGCGGATA | 133 |
| C20 orf1 | NM_012112 | S3561/C20 or.r1 | ACGGTCCTAGGTTTGAGGTTAAGA | 134 |
| C20 orf1 | NM_012112 | S3562/C20 or.p1 | CAGGTCCCATTGCCGGGCG | 135 |
| CA9 | NM_001216 | S1398/CA9.f3 | ATCCTAGCCCTGGTTTTTGG | 136 |
| CA9 | NM_001216 | S1399/CA9.r3 | CTGCCTTCTCATCTGCACAA | 137 |
| CA9 | NM_001216 | S4938/CA9.p3 | TTTGCTGTCACCAGCGTCGC | 138 |
| CALD1 | NM_004342 | S4683/CALD1.f2 | CACTAAGGTTTGAGACAGTTCCAGAA | 139 |
| CALD1 | NM_004342 | S4684/CALD1.r2 | GCGAATTAGCCCTCTACAACTGA | 140 |
| CALD1 | NM_004342 | S4685/CALD1.p2 | AACCCAAGCTCAAGACGCAGGACGAG | 141 |
| CAPZA1 | NM_006135 | T2228/CAPZA1.f1 | TCGTTGGAGATCAGAGTGGA | 142 |
| CAPZA1 | NM_006135 | T2229/CAPZA1.r1 | TTAAGCACGCCAACCACC | 143 |
| CAPZA1 | NM_006135 | T2230/CAPZA1.p1 | TCACCATCACACCACCTACAGCCC | 144 |
| CAV1 | NM_001753 | S7151/CAV1.f1 | GTGGCTCAACATTGTGTTCC | 145 |
| CAV1 | NM_001753 | S7152/CAV1.r1 | CAATGGCCTCCATTTTACAG | 146 |
| CAV1 | NM_001753 | S7153/CAV1.p1 | ATTTCAGCTGATCAGTGGGCCTCC | 147 |
| CCNB1 | NM_031966 | S1720/CCNB1.f2 | TTCAGGTTGTTGCAGGAGAC | 148 |
| CCNB1 | NM_031966 | S1721/CCNB1.r2 | CATCTTCTTGGGCACACAAT | 149 |
| CCNB1 | NM_031966 | S4733/CCNB1.p2 | TGTCTCCATTATTGATCGGTTCATGCA | 150 |
| CCND1 | NM_053056 | S0058/CCND1.f3 | GCATGTTCGTGGCCTCTAAGA | 151 |
| CCND1 | NM_053056 | S0060/CCND1.r3 | CGGTGTAGATGCACAGCTTCTC | 152 |
| CCND1 | NM_053056 | S4986/CCND1.p3 | AAGGAGACCATCCCCCTGACGGC | 153 |
| CCNE2 | NM_057749 | S1458/CCNES.f2 | ATGCTGTGGCTCCTTCCTAACT | 154 |
| CCNE2 | NM_057749 | S1459/CCNE2.r2 | ACCCAAATTGTGATATACAAAAAGGTT | 155 |
| CCNE2 | NM_057749 | S4945/CCNE2.p2 | TACCAAGCAACCTACATGTCAAGAAAGCCC | 156 |
| CCT3 | NM_001008800 | T1053/CCT3.f1 | ATCCAAGGCCATGACTGG | 157 |
| CCT3 | NM_001008800 | T1054/CCT3.r1 | GGAATGACCTCTAGGGCCTG | 158 |
| CCT3 | NM_001008800 | T1055/CCT3.p1 | ACAGCCCTGTATGGCCATTGTTCC | 159 |
| CD14 | NM_000591 | T1997/CD14.f1 | GTGTGCTAGCGTACTCCCG | 160 |
| CD14 | NM_000591 | T1998/CD14.r1 | GCATGGTGCCGGTTATCT | 161 |
| CD14 | NM_000591 | T1999/CD14.p1 | CAAGGAACTGACGCTCGAGGACCT | 162 |
| CD31 | NM_000442 | S1407/CD31.f3 | TGTATTTCAAGACCTCTGTGCACTT | 163 |
| CD31 | NM_000442 | S1408/CD31.r3 | TTAGCCTGAGGAATTGCTGTGTT | 164 |
| CD31 | NM_000442 | S4939/CD31.p3 | TTTATGAACCTGCCCTGCTCCCACA | 165 |
| CD3z | NM_000734 | S0064/CD3z.f1 | AGATGAAGTGGAAGGCGCTT | 166 |
| CD3z | NM_000734 | S0066/CD3z.r1 | TGCCTCTGTAATCGGCAACTG | 167 |
| CD3z | NM_000734 | S4988/CD3z.p1 | CACCGCGGCCATCCTGCA | 168 |
| CD63 | NM_001780 | T1988/CD63.f1 | AGTGGGACTGATTGCCGT | 169 |
| CD63 | NM_001780 | T1989/CD63.r1 | GGGTAGCCCCCTGGATTAT | 170 |
| CD63 | NM_001780 | T1990/CD63.p1 | TCTGACTCAGGACAAGCTGTGCCC | 171 |
| CD68 | NM_001251 | S0067/CD68.f2 | TGGTTCCCAGCCCTGTGT | 172 |
| CD68 | NM_001251 | S0069/CD68.r2 | CTCCTCCACCCTGGGTTGT | 173 |
| CD68 | NM_001251 | S4734/CD68.p2 | CTCCAAGCCCAGATTCAGATTCGAGTCA | 174 |
| CDC2 | NM_001786 | S7238/CDC2.f1 | GAGAGCGACGCGGTTGTT | 175 |
| CDC2 | NM_001786 | S7239/CDC2.r1 | GTATGGTAGATCCCGGCTTATTATTC | 176 |
| CDC2 | NM_001786 | S7240/CDC2.p1 | TAGCTGCCGCTGCGGCCG | 177 |
| CDC20 | NM_001255 | S4447/CDC20.f1 | TGGATTGGAGTTCTGGGAATG | 178 |
| CDC20 | NM_001255 | S4448/CDC20.r1 | GCTTGCACTCCACAGGTACACA | 179 |
| CDC20 | NM_001255 | S4449/CDC20.p1 | ACTGGCCGTGGCACTGGACAACA | 180 |
| CDC25B | NM_021873 | S1160/CDC25B.f1 | AAACGAGCAGTTTGCCATCAG | 181 |
| CDC25B | NM_021873 | S1161/CDC25B.r1 | GTTGGTGATGTTCCGAAGCA | 182 |
| CDC25B | NM_021873 | S4842/CDC25B.p1 | CCTCACCGGCATAGACTGGAAGCG | 183 |
| CDCA8 | NM_018101 | T2060/CDCA8.f1 | GAGGCACAGTATTGCCCAG | 184 |
| CDCA8 | NM_018101 | T2061/CDCA8.r1 | GAGACGGTTGGAGAGCTTCTT | 185 |
| CDCA8 | NM_019101 | T2062/CDCA8.p1 | ATGTTTCCCAAGGCCTCTGGATCC | 186 |
| CDH1 | NM_004360 | S0073/CDH1.f3 | TGAGTGTCCCCCGGTATCTTC | 187 |
| CDH1 | NM_004360 | S0075/CDH1.r3 | CAGCCGCTTTCAGATTTTCAT | 188 |
| CDH1 | NM_004360 | S4990/CDH1.p3 | TGCCAATCCCGATGAAATTGGAAATTT | 189 |
| CDK5 | NM_004935 | T2000/CDK5.f1 | AAGCCCTATCCGATGTACCC | 190 |
| CDK5 | NM_004935 | T2001/CDK5.r1 | CTGTGGCATTGAGTTTGGG | 191 |
| CDK5 | NM_004935 | T2002/CDK5.p1 | CACAACATCCCTGGTGAACGTCGT | 192 |
| CDKN1C | NM_000076 | T2003/CDKN1C.f1 | CGGCGATCAAGAAGCTGT | 193 |
| CDKN1C | NM_000076 | T2004/CDKN1C.r1 | CAGGCGCTGATCTCTTGC | 194 |
| CDKN1C | NM_000076 | T2005/CDKN1C.p1 | CGGGCCTCTGATCTCCGATTTCTT | 195 |
| CEGP1 | NM_020974 | S1494/CEGP1.f2 | TGACAATCAGCACACCTGCAT | 196 |
| CEGP1 | NM_020974 | S1495/CEGP1.r2 | TGTGACTACAGCCGTGATCCTTA | 197 |
| CEGP1 | NM_020974 | S4735/CEGP1.p2 | CAGGCCCTCTTCCGAGCGGT | 198 |
| CENPA | NM_001809 | S7082/CENPA.f1 | TAAATTCACTCGTGGTGTGGA | 199 |
| CENPA | NM_001809 | S7083/CENPA.r1 | GCCTCTTGTAGGGCCAATAG | 200 |
| CENPA | NM_001809 | S7084/CENPA.p1 | CTTCAATTGGCAAGCCCAGGC | 201 |
| CENPE | NM_001813 | S5496/CENPE.f3 | GGATGCTGGTGACCTCTTCT | 202 |
| CENPE | NM_001813 | S5497/CENPE.r3 | GCCAAGGCACCAAGTAACTC | 203 |
| CENPE | NM_001813 | S5498/CENPE.p3 | TCCCTCACGTTGCAACAGGAATTAA | 204 |
| CENPF | NM_016343 | S9200/CENPF.f1 | CTCCCGTCAACAGCGTTC | 205 |
| CENPF | NM_016343 | S9201/CENPF.r1 | GGGTGAGTCTGGCCTTCA | 206 |
| CENPF | NM_016343 | S9202/CENPF.p1 | ACACTGGACCAGGAGTGCATCCAG | 207 |
| CGA (CHGA official) | NM_001275 | S3221/CGA (C.f3 | CTGAAGGAGCTCCAAGACCT | 208 |
| CGA (CHGA official) | NM_001275 | S3222/CGA (C.r3 | CAAAACCGCTGTGTTTCTTC | 209 |
| CGA (CHGA official) | NM_001275 | S3254/CGA (C.p3 | TGCTGATGTGCCCTCTCCTTGG | 210 |
| CHFR | NM_018223 | S7085/CHFR.f1 | AAGGAAGTGGTCCCTCTGTG | 211 |
| CHFR | NM_018223 | S7086/CHFR.r1 | GACGCAGTCTTTCTGTCTGG | 212 |
| CHFR | NM_018223 | S7087/CHFR.p1 | TGAAGTCTCCAGCTTTGCCTCAGC | 213 |
| Chk1 | NM_001274 | S1422/Chk1.f2 | GATAAATTGGTACAAGGGATCAGCTT | 214 |
| Chk1 | NM_001274 | S1423/Chk1.r2 | GGGTGCCAAGTAACTGACTATTCA | 215 |
| Chk1 | NM_001274 | S4941/Chk1.p2 | CCAGCCCACATGTCCTGATCATATGC | 216 |
| Chk2 | NM_007194 | S1434/Chk2.f3 | ATGTGGAACCCCCACCTACTT | 217 |
| Chk2 | NM_007194 | S1435/Chk2.r3 | CAGTCCACAGCACGGTTATACC | 218 |
| Chk2 | NM_007194 | S4942/Chk2.p3 | AGTCCCAACAGAAACAAGAACTTCAGGCG | 219 |
| cIAP2 | NM_001165 | S0076/cIAP2.f2 | GGATATTTCCGTGGCTCTTATTCA | 220 |
| cIAP2 | NM_001165 | S0078/cIAP2.r2 | CTTCTCATCAAGGCAGAAAAATCTT | 221 |
| cIAP2 | NM_001165 | S4991/cIAP2.p2 | TCTCCATCAAATCCTGTAAACTCCAGAGCA | 222 |
| CKAP1 | NM_001281 | T2293/CKAP1.f1 | TCATTGACCACAGTGGCG | 223 |
| CKAP1 | NM_001281 | T2294/CKAP1.r1 | TCGTGTACTTCTCCACCCG | 224 |
| CKAP1 | NM_001281 | T2295/CKAP1.p1 | CACGTCCTCATACTCACCAAGGCG | 225 |
| CLU | NM_001831 | S5666/CLU.f3 | CCCCAGGATACCTACCACTACCT | 226 |
| CLU | NM_001831 | S5667/CLU.r3 | TGCGGGACTTGGGAAAGA | 227 |
| CLU | NM_001831 | S5668/CLU.p3 | CCCTTCAGCCTGCCCCACCG | 228 |
| cMet | NM_000245 | S0082/cMet.f2 | GACATTTCCAGTCCTGCAGTCA | 229 |
| cMet | NM_000245 | S0084/cMet.r2 | CTCCGATCGCACACATTTGT | 230 |
| cMet | NM_000245 | S4993/cMet.p2 | TGCCTCTCTGCCCCACCCTTTGT | 231 |
| cMYC | NM_002467 | S0085/cMYC.f3 | TCCCTCCACTCGGAAGGACTA | 232 |
| cMYC | NM_002467 | S0087/cMYC.r3 | CGGTTGTTGCTGATCTGTCTCA | 233 |
| cMYC | NM_002467 | S4994/cMYC.p3 | TCTGACACTGTCCAACTTGACCCTCTT | 234 |
| CNN | NM_001299 | S4564/CNN.f1 | TCCACCCTCCTGGCTTTG | 234 |
| CNN | NM_001299 | S4565/CNN.r1 | TCACTCCCACGTTCACCTTGT | 236 |
| CNN | NM_001299 | S4566/CNN.p1 | TCCTTTCGTCTTCGCCATGCTGG | 237 |
| COL1A1 | NM_000088 | S4531/COL1A1.f1 | GTGGCCATCCAGCTGACC | 238 |
| COL1A1 | NM_000088 | S4532/COL1A1.r1 | CAGTGGTAGGTGATGTTCTGGGA | 239 |
| COL1A1 | NM_000088 | S4533/COL1A1.p1 | TCCTGCGCCTGATGTCCACCG | 240 |
| COL1A2 | NM_000089 | S4534/COL1A2.f1 | CAGCCAAGAACTGGTATAGGAGCT | 241 |
| COL1A2 | NM_000089 | S4535/COL1A2.r1 | AAACTGGCTGCCAGCATTG | 242 |
| COL1A2 | NM_000089 | S4536/COL1A2.p1 | TCTCCTAGCCAGACGTGTTTCTTGTCCTTG | 243 |
| COL6A3 | NM_004369 | T1062/COL6A3.f1 | GAGAGCAAGCGAGACATTCTG | 244 |
| COL6A3 | NM_004369 | T1063/COL6A3.r1 | AACAGGGAACTGGCCCAC | 245 |
| COL6A3 | NM_004369 | T1064/COL6A3.p1 | CCTCTTTGACGGCTCAGCCAATCT | 246 |
| Contig 51037 | NM_198477 | S2070/Contig.f1 | CGACAGTTGCGATGAAAGTTCTAA | 247 |
| Contig 51037 | NM_198477 | S2071/Contig.r1 | GGCTGCTAGAGACCATGGACAT | 248 |
| Contig 51037 | NM_198477 | S5059/Contig.p1 | CCTCCTCCTGTTGCTGCCACTAATGCT | 249 |
| COX2 | NM_000963 | S0088/COX2.f1 | TCTGCAGAGTTGGAAGCACTCTA | 250 |
| COX2 | NM_000963 | S0090/COX2.r1 | GCCGAGGCTTTTCTACCAGAA | 251 |
| COX2 | NM_000963 | S4995/COX2.p1 | CAGGATACAGCTCCACAGCATCGATGTC | 252 |
| COX7C | NM_001867 | T0219/COX7C.f1 | ACCTCTGTGGTCCGTAGGAG | 253 |
| COX7C | NM_001867 | T0220/COX7C.r1 | CGACCACTTGTTTTCCACTG | 254 |
| COX7C | NM_001867 | T0221/COX7C.p1 | TCTTCCCAGGGCCCTCCTCATAGT | 255 |
| CRABP1 | NM_004378 | S5441/CRABP1.f3 | AACTTCAAGGTCGGAGAAGG | 256 |
| CRABP1 | NM_004378 | S5442/CRABP1.r3 | TGGCTAAACTCCTGCACTTG | 257 |
| CRABP1 | NM_004378 | S5443/CRABP1.p3 | CCGTCCACGGTCTCCTCCTCA | 258 |
| CRIP2 | NM_001312 | S5676/CRIP2.f3 | GTGCTACGCCACCCTGTT | 259 |
| CRIP2 | NM_001312 | S5677/CRIP2.r3 | CAGGGGCTTCTCGTAGATGT | 260 |
| CRIP2 | NM_001312 | S5678/CRIP2.p3 | CCGATGTTCACGCCTTTGGGTC | 261 |
| CRYAB | NM_001885 | S8302/CRYAB.f1 | GATGTGATTGAGGTGCATGG | 262 |
| CRYAB | NM_001885 | S8303/CRYAB.r1 | GAACTCCCTGGAGATGAAACC | 263 |
| CRYAB | NM_001885 | S8304/CRYAB.p1 | TGTTCATCCTGGCGCTCTTCATGT | 264 |
| CSF1 | NM_000757 | S1482/CSF1.f1 | TGCAGCGGCTGATTGACA | 265 |
| CSF1 | NM_000757 | S1483/CSF1.r1 | CAACTGTTCCTGGTCTACAAACTCA | 266 |
| CSF1 | NM_000757 | S4948/CSF1.p1 | TCAGATGGAGACCTCGTGCCAAATTACA | 267 |
| CSNK1D | NM_001893 | S2332/CSNK1D.f3 | AGCTTTTCCGGAATCTGTTC | 268 |
| CSNK1D | NM_001893 | S2333/CSNK1D.r3 | ATTTGAGCATGTTCCAGTCG | 269 |
| CSNK1D | NM_001893 | S4850/CSNK1D.p3 | CATCGCCAGGGCTTCTCCTATGAC | 270 |
| CST7 | NM_003650 | T2108/CST7.f1 | TGGCAGAACTACCTGCAAGA | 271 |
| CST7 | NM_003650 | T2109/CST7.r1 | TGCTTCAAGGTGTGGTTGG | 272 |
| CST7 | NM_003650 | T2110/CST7.p1 | CACCTGCGTCTGGATGACTGTGAC | 273 |
| CTSD | NM_001909 | S1152/CTSD.f2 | GTACATGATCCCCTGTGAGAAGGT | 274 |
| CTSD | NM_001909 | S1153/CTSD.r2 | GGGACAGCTTGTAGCCTTTGC | 275 |
| CTSD | NM_001909 | S4841/CTSD.p2 | ACCCTGCCCGCGATCACACTGA | 276 |
| CTSL | NM_001912 | S1303/CTSL.f2 | GGGAGGCTTATCTCACTGAGTGA | 277 |
| CTSL | NM_001912 | S1304/CTSL.r2 | CCATTGCAGCCTTCATTGC | 278 |
| CTSL | NM_001912 | S4899/CTSL.p2 | TTGAGGCCCAGAGCAGTCTACCAGATTCT | 279 |
| CTSL2 | NM_001333 | S4354/CTSL2.f1 | TGTCTCACTGAGCGAGCAGAA | 280 |
| CTSL2 | NM_001333 | S4355/CTSL2.r1 | ACCATTGCAGCCCTGATTG | 281 |
| CTSL2 | NM_001333 | S4356/CTSL2.p1 | CTTGAGGACGCGAACAGTCCACCA | 282 |
| CXCR4 | NM_003467 | S5966/CXCR4.f3 | TGACCGCTTCTACCCCAATG | 283 |
| CXCR4 | NM_003467 | S5967/CXCR4.r3 | AGGATAAGGCCAACCATGATGT | 284 |
| CXCR4 | NM_003467 | S5968/CXCR4.p3 | CTGAAACTGGAACACAACCACCCACAAG | 285 |
| CYBA | NM_000101 | S5300/CYBA.f1 | GGTGCCTACTCCATTGTGG | 286 |
| CYBA | NM_000101 | S5301/CYBA.r1 | GTGGAGCCCTTCTTCCTCTT | 287 |
| CYBA | NM_000101 | S5302/CYBA.p1 | TACTCCAGCAGGCACACAAACACG | 288 |
| CYP1B1 | NM_000104 | S0094/CYP1B1.f3 | CCAGCTTTGTGCCTGTCACTAT | 289 |
| CYP1B1 | NM_000104 | S0096/CYP1B1.r3 | GGGAATGTGGTAGCCCAAGA | 290 |
| CYP1B1 | NM_000104 | S4996/CYP1B1.p3 | CTCATGCCACCACTGCCAACACCTC | 291 |
| CYP2C8 | NM_000770 | S1470/CYP2C8.f2 | CCGTGTTCAAGAGGAAGCTC | 292 |
| CYP2C8 | NM_000770 | S1471/CYP2C8.r2 | AGTGGGATCACAGGGTGAAG | 293 |
| CYP2C8 | NM_000770 | S4946/CYP2C8.p2 | TTTTCTCAACTCCTCCACAAGGCA | 294 |
| CYP3A4 | NM_017460 | S1620/CYP3A4.f2 | AGAACAAGGACAACATAGATCCTTACATAT | 295 |
| CYP3A4 | NM_017460 | S1621/CYP3A4.r2 | GCAAACCTCATGCCAATGC | 296 |
| CYP3A4 | NM_017460 | S4906/CYP3A4.p2 | CACACCCTTTGGAAGTGGACCCAGAA | 297 |
| DDR1 | NM_001954 | T2156/DDR1.f1 | CCGTGTGGCTCGCTTTCT | 298 |
| DDR1 | NM_001954 | T2157/DDR1.r1 | GGAGATTTCGCTGAAGAGTAACCA | 299 |
| DDR1 | NM_001954 | T2158/DDR1.p1 | TGCCGCTTCCTCTTTGCGGG | 300 |
| DIABLO | NM_019887 | S0808/DIABLO.f1 | CACAATGGCGGCTCTGAAG | 301 |
| DIABLO | NM_019887 | S0809/DIABLO.r1 | ACACAAACACTGTCTGTACCTGAAGA | 302 |
| DIABLO | NM_019887 | S4813/DIABLO.p1 | AAGTTACGCTGCGCGACAGCCAA | 303 |
| DIAPH1 | NM_005219 | S7608/DIAPH1.f1 | CAAGCAGTCAAGGAGAACCA | 304 |
| DIAPH1 | NM_005219 | S7609/DIAPH1.r1 | AGTTTTGCTCGCCTCATCTT | 305 |
| DIAPH1 | NM_005219 | S7610/DIAPH1.p1 | TTCTTCTGTCTCCCGCCGCTTC | 306 |
| DICER1 | NM_177438 | S5294/DICER1.f2 | TCCAATTCCAGCATCACTGT | 307 |
| DICER1 | NM_177438 | S5295/DICER1.r2 | GGCAGTGAAGGCGATAAAGT | 308 |
| DICER1 | NM_177438 | S5296/DICER1.p2 | AGAAAAGCTGTTTGTCTCCCCAGCA | 309 |
| DKFZp564D0462; | NM_198569 | S4405/DKFZp5.f2 | CAGTGCTTCCATGGACAAGT | 310 |
| DKFZp564D0462; | NM_198569 | S4406/DKFZp5.r2 | TGGACAGGGATGATTGATGT | 311 |
| DKFZp564D0462; | NM_198569 | S4407/DKFZp5.p2 | ATCTCCATCAGCATGGGCCAGTTT | 312 |
| DR4 | NM_003844 | S2532/DR4.f2 | TGCACAGAGGGTGTGGGTTAC | 313 |
| DR4 | NM_003844 | S2533/DR4.r2 | TCTTCATCTGATTTACAAGCTGTACATG | 314 |
| DR4 | NM_003844 | S4981/DR4.p2 | CAATGCTTCCAACAATTTGTTTGCTTGCC | 315 |
| DR5 | NM_003842 | S2551/DR5.f2 | CTCTGAGACAGTGCTTCGATGACT | 316 |
| DR5 | NM_003842 | S2552/DR5.r2 | CCATGAGGCCCAACTTCCT | 317 |
| DR5 | NM_003842 | S4979/DR5.p2 | CAGACTTGGTGCCCTTTGACTCC | 318 |
| DUSP1 | NM_004417 | S7476/DUSP1.f1 | AGACATCAGCTCCTGGTTCA | 319 |
| DUSP1 | NM_004417 | S7477/DUSP1.r1 | GACAAACACCCTTCCTCCAG | 320 |
| DUSP1 | NM_004417 | S7478/DUSP1.p1 | CGAGGCCATTGACTTCATAGACTCCA | 321 |
| EEF1D | NM_001960 | T2159/EEF1D.f1 | CAGAGGATGACGAGGATGATGA | 322 |
| EEF1D | NM_001960 | T2160/EEF1D.r1 | CTGTGCCGCCTCCTTGTC | 323 |
| EEF1D | NM_001960 | T2161/EEF1D.p1 | CTCCTCATTGTCACTGCCAAACAGGTCA | 324 |
| EGFR | NM_005228 | S0103/EGFR.f2 | TGTCGATGGACTTCCAGAAC | 325 |
| EGFR | NM_005228 | S0105/EGFR.r2 | ATTGGGACAGCTTGGATCA | 326 |
| EGFR | NM_005228 | S4999/EGFR.p2 | CACCTGGGCAGCTGCCAA | 327 |
| EIF4E | NM_001968 | S0106/EIF4E.f1 | GATCTAAGATGGCGACTGTCGAA | 328 |
| EIF4E | NM_001968 | S0108/EIF4E.r1 | TTAGATTCCGTTTTCTCCTCTTCTG | 329 |
| EIF4E | NM_001968 | S5000/EIF4E.p1 | ACCACCCCTACTCCTAATCCCCCGACT | 330 |
| EIF4EL3 | NM_004846 | S4495/EIF4EL.f1 | AAGCCGCGGTTGAATGTG | 331 |
| EIF4EL3 | NM_004846 | S4496/EIF4EL.r1 | TGACGCCAGCTTCAATGATG | 332 |
| EIF4EL3 | NM_004846 | S4497/EIF4EL.p1 | TGACCCTCTCCCTCTCTGGATGGCA | 333 |
| ELP3 | NM_018091 | T2234/ELP3.f1 | CTCGGATCCTAGCCCTCG | 334 |
| ELP3 | NM_018091 | T2235/ELP3.r1 | GGCATTGGAATATCCCTCTGTA | 335 |
| ELP3 | NM_018091 | T2236/ELP3.p1 | CCTCCATGGACTCGAGTGTACCGA | 336 |
| ER2 | NM_001437 | S0109/ER2.f2 | TGGTCCATCGCCAGTTATCA | 337 |
| ER2 | NM_001437 | S0111/ER2.r2 | TGTTCTAGCGATCTTGCTTCACA | 338 |
| ER2 | NM_001437 | S5001/ER2.p2 | ATCTGTATGCGGAACCTCAAAAGAGTCCCT | 339 |
| ErbB3 | NM_001982 | S0112/ErbB3.f1 | CGGTTATGTCATGCCAGATACAC | 340 |
| ErbB3 | NM_001982 | S0114/ErbB3.r1 | GAACTGAGACCCACTGAAGAAAGG | 341 |
| ErbB3 | NM_001982 | S5002/ErbB3.p1 | CCTCAAAGGTACTCCCTCCTCCCGG | 342 |
| ERBB4 | NM_005235 | S1231/ERBB4.f3 | TGGCTCTTAATCAGTTTCGTTACCT | 343 |
| ERBB4 | NM_005235 | S1232/ERBB4.r3 | CAAGGCATATCGATCCTCATAAAGT | 344 |
| ERBB4 | NM_005235 | S4891/ERBB4.p3 | TGTCCCACGAATAATGCGTAAATTCTCCAG | 345 |
| ERCC1 | NM_001983 | S2437/ERCC1.f2 | GTCCAGGTGGATGTGAAAGA | 346 |
| ERCC1 | NM_001983 | S2438/ERCC1.r2 | CGGCCAGGATACACATCTTA | 347 |
| ERCC1 | NM_001983 | S4920/ERCC1.p2 | CAGCAGGCCCTCAAGGAGCTG | 348 |
| ERK1 | NM_002746 | S1560/ERK1.f3 | ACGGATCACAGTGGAGGAAG | 349 |
| ERK1 | NM_002746 | S1561/ERK1.r3 | CTCATCCGTCGGGTCATAGT | 350 |
| ERK1 | NM_002746 | S4882/ERK1.p3 | CGCTGGCTCACCCCTACCTG | 351 |
| ESPL1 | NM_012291 | S5686/ESPL1.f3 | ACCCCCAGACCGGATCAG | 352 |
| ESPL1 | NM_012291 | S5687/ESPL1.r3 | TGTAGGGCAGACTTCCTCAAACA | 353 |
| ESPL1 | NM_012291 | S5688/ESPL1.p3 | CTGGCCCTCATGTCCCCTTCACG | 354 |
| EstR1 | NM_000125 | S0115/EstR1.f1 | CGTGGTGCCCCTCTATGAC | 355 |
| EstR1 | NM_000125 | S0117/EstR1.r1 | GGCTAGTGGGCGCATGTAG | 356 |
| EstR1 | NM_000125 | S4737/EstR1.p1 | CTGGAGATGCTGGACGCCC | 357 |
| fas | NM_000043 | S0118/fas.f1 | GGATTGCTCAACAACCATGCT | 358 |
| fas | NM_000043 | S0120/fas.r1 | GGCATTAACACTTTTGGACGATAA | 359 |
| fas | NM_000043 | S5003/fas.p1 | TCTGGACCCTCCTACCTCTGGTTCTTACGT | 360 |
| fasI | NM_000639 | S0121/fasI.f2 | GCACTTTGGGATTCTTTCCATTAT | 361 |
| fasI | NM_000639 | S0123/fasI.r2 | GCATGTAAGAAGACCCTCACTGAA | 362 |
| fasI | NM_000639 | S5004/fasI.p2 | ACAACATTCTCGGTGCCTGTAACAAAGAA | 363 |
| FASN | NM_004104 | S8287/FASN.f1 | GCCTCTTCCTGTTCGACG | 364 |
| FASN | NM_004104 | S8288/FASN.r1 | GCTTTGCCCGGTAGCTCT | 365 |
| FASN | NM_004104 | S8289/FASN.p1 | TCGCCCACCTACGTACTGGCCTAC | 366 |
| FBXO5 | NM_012177 | S2017/FBXO5.r1 | GGATTGTAGACTGTCACCGAAATTC | 367 |
| FBXO5 | NM_012177 | S2018/FBXO5.f1 | GGCTATTCCTCATTTTCTCTACAAAGTG | 368 |
| FBXO5 | NM_012177 | S5061/FBXO5.p1 | CCTCCAGGAGGCTACCTTCTTCATGTTCAC | 369 |
| FDFT1 | NM_004462 | T2006/FDFT1.f1 | AAGGAAAGGGTGCCTCATC | 370 |
| FDFT1 | NM_004462 | T2007/FDFT1.r1 | GAGCCACAAGCAGCACAGT | 371 |
| FDFT1 | NM_004462 | T2008/FDFT1.p1 | CATCACCCACAAGGACAGGTTGCT | 372 |
| FGFR1 | NM_023109 | S0818/FGFR1.f3 | CACGGGACATTCACCACATC | 373 |
| FGFR1 | NM_023109 | S0819/FGFR1.r3 | GGGTGCCATCCACTTCACA | 374 |
| FGFR1 | NM_023109 | S4816/FGFR1.p3 | ATAAAAAGACAACCAACGGCCGACTGC | 375 |
| FHIT | NM_002012 | S2443/FHIT.f1 | CCAGTGGAGCGCTTCCAT | 376 |
| FHIT | NM_002012 | S2444/FHIT.r1 | CTCTCTGGGTCGTCTGAAACAA | 377 |
| FHIT | NM_002012 | S4921/FHIT.p1 | TCGGCCACTTCATCAGGACGCAG | 378 |
| FIGF | NM_004469 | S8941/FIGF.f1 | GGTTCCAGCTTTCTGTAGCTGT | 379 |
| FIGF | NM_004469 | S8942/FIGF.r1 | GCCGCAGGTTCTAGTTGCT | 380 |
| FIGF | NM_004469 | S8943/FIGF.p1 | ATTGGTGGCCACACCACCTCCTTA | 381 |
| FLJ20354 | NM_017779 | S4309/FLJ203.f1 | GCGTATGATTTCCCGAATGAG | 382 |
| (DEPDC1 official) | | | | |
| FLJ20354 | NM_017779 | S4310/FLJ203.r1 | CAGTGACCTCGTACCCATTGC | 383 |
| (DEPDC1 official) | | | | |
| FLJ20354 | NM_017779 | S4311/FLJ203.p1 | ATGTTGATATGCCCAAACTTCATGA | 384 |
| (DEPDC1 official) | | | | |
| FOS | NM_005252 | S6726/FOS.f1 | CGAGCCCTTTGATGACTTCCT | 385 |
| FOS | NM_005252 | S6727/FOS.r1 | GGAGCGGGCTGTCTCAGA | 386 |
| FOS | NM_005252 | S6728/FOS.p1 | TCCCAGCATCATCCAGGCCCAG | 387 |
| FOXM1 | NM_021953 | S2006/FOXM1.f1 | CCACCCCGAGCAAATCTGT | 388 |
| FOXM1 | NM_021953 | S2007/FOXM1.r1 | AAATCCAGTCCCCCTACTTTGG | 389 |
| FOXM1 | NM_021953 | S4757/FOXM1.p1 | CCTGAATCCTGGAGGCTCACGCC | 390 |
| FUS | NM_004960 | S2936/FUS.f1 | GGATAATTCAGACAACAACACCATCT | 391 |
| FUS | NM_004960 | S2937/FUS.r1 | TGAAGTAATCAGCCACAGACTCAAT | 392 |
| FUS | NM_004960 | S4801/FUS.p1 | TCAATTGTAACATTCTCACCCAGGCCTTG | 393 |
| FYN | NM_002037 | S5695/FYN.f3 | GAAGCGCAGATCATGAAGAA | 394 |
| FYN | NM_002037 | S5696/FYN.r3 | CTCCTCAGACACCACTGCAT | 395 |
| FYN | NM_002037 | S5697/FYN.p3 | CTGAAGCACGACAAGCTGGTCCAG | 396 |
| G1P3 | NM_002038 | T1086/F1P3.f1 | CCTCCAACTCCTAGCCTCAA | 397 |
| G1P3 | NM_002038 | T1087/F1P3.r1 | GGCGCATGCTTGTAATCC | 398 |
| G1P3 | NM_002038 | T1088/F1P3.p1 | TGATCCTCCTGTCTCAACCTCCCA | 399 |
| GADD45 | NM_001924 | S5835/GADD45.f3 | GTGCTGGTGACGAATCCA | 400 |
| GADD45 | NM_001924 | S5836/GADD45.r3 | CCCGGCAAAAACAAATAAGT | 401 |
| GADD45 | NM_001924 | S5837/GADD45.p3 | TTCATCTCAATGGAAGGATCCTGCC | 402 |
| GADD45B | NM_015675 | S6929/GADD45.f1 | ACCCTCGACAAGACCACACT | 403 |
| GADD45B | NM_015675 | S6930/GADD45.r1 | TGGGAGTTCATGGGTACAGA | 404 |
| GADD45B | NM_015675 | S6931/GADD45.p1 | AACTTCAGCCCCAGCTCCCAAGTC | 405 |
| GAGE1 | NM_001468 | T2162/GAGE1.f1 | AAGGGCAATCACAGTGTTAAAAGAA | 406 |
| GAGE1 | NM_001468 | T2163/GAGE1.r1 | GGAGAACTTCAATGAAGAATTTTCCA | 407 |
| GAGE1 | NM_001468 | T2164/GAGE1.p1 | CATAGGAGCAGCCTGCAACATTTCAGCAT | 408 |
| GAPDH | NM_002046 | S0374/GAPDH.f1 | ATTCCACCCATGGCAAATTC | 409 |
| GAPDH | NM_002046 | S0375/GAPDH.r1 | GATGGGATTTCCATTGATGACA | 410 |
| GAPDH | NM_002046 | S4738/GAPDH.p1 | CCGTTCTCAGCCTTGACGGTGC | 411 |
| GATA3 | NM_002051 | S0127/GATA3.f3 | CAAAGGAGCTCACTGTGGTGTCT | 412 |
| GATA3 | NM_002051 | S0129/GATA3.r3 | GAGTCAGAATGGCTTATTCACAGATG | 413 |
| GATA3 | NM_002051 | S5005/GATA3.p3 | TGTTCCAACCACTGAATCTGGACC | 414 |
| GBP1 | NM_002053 | S5698/GBP1.f1 | TTGGGAAATATTTGGGCATT | 415 |
| GBP1 | NM_002053 | S5699/GBP1.r1 | AGAAGCTAGGGTGGTTGTCC | 416 |
| GBP1 | NM_002053 | S5700/GBP1.p1 | TTGGGACATTGTAGACTTGGCCAGAC | 417 |
| GBP2 | NM_004120 | S5707/GBP2.f2 | GCATGGGAACCATCAACCA | 418 |
| GBP2 | NM_004120 | S5708/GBP2.r2 | TGAGGAGTTTGCCTTGATTCG | 419 |
| GBP2 | NM_004120 | S5709/GBP2.p2 | CCATGGACCAACTTCACTATGTGACAGAGC | 420 |
| GCLC | NM_001498 | S0772/GCLC.f3 | CTGTTGCAGGAAGGCATTGA | 421 |
| GCLC | NM_001498 | S0773/GCLC.r3 | GTCAGTGGGTCTCTAATAAAGAGATGAG | 422 |
| GCLC | NM_001498 | S4803/GCLC.p3 | CATCTCCTGGCCCAGCATGTT | 423 |
| GDF15 | NM_004864 | S7806/GDF15.f1 | CGCTCCAGACCTATGATGACT | 424 |
| GDF15 | NM_004864 | S7807/GDF15.r1 | ACAGTGGAAGGACCAGGACT | 425 |
| GDF15 | NM_004864 | S7808/GDF15.p1 | TGTTAGCCAAAGACTGCCACTGCA | 426 |
| GGPS1 | NM_004837 | S1590/GGPS1.f1 | CTCCGACGTGGCTTTCCA | 427 |
| GGPS1 | NM_004837 | S1591/GGPS1.r1 | CGTAATTGGCAGAATTGATGACA | 428 |
| GGPS1 | NM_004837 | S4896/GGPS1.p1 | TGGCCCACAGCATCTATGGAATCCC | 429 |
| GLRX | NM_002064 | T2165/GLRX.f1 | GGAGCTCTGCAGTAACCACAGAA | 430 |
| GLRX | NM_002064 | T2166/GLRX.r1 | CAATGCCATCCAGCTCTTGA | 431 |
| GLRX | NM_002064 | T2167/GLRX.p1 | AGGCCCCATGCTGACGTCCCTC | 432 |
| GNS | NM_002076 | T2009/GNS.f1 | GGTGAAGGTTGTCTCTTCCG | 433 |
| GNS | NM_002076 | T2010/GNS.r1 | CAGCCCTTCCACTTGTCTG | 434 |
| GNS | NM_002076 | T2011/GNS.p1 | AAGAGCCCTGTCTTCAGAAGGCCC | 435 |
| GPR56 | NM_005682 | T2120/GPR56.f1 | TACCCTTCCATGTGCTGGAT | 436 |
| GPR56 | NM_005682 | T2121/GPR56.r1 | GCTGAAGAGGCCCAGGTT | 437 |
| GPR56 | NM_005682 | T2122/GPR56.p1 | CGGGACTCCCTGGTCAGCTACATC | 438 |
| GPX1 | NM_000581 | S8296/GPX1.f2 | GCTTATGACCGACCCCAA | 439 |
| GPX1 | NM_000581 | S8297/GPX1.r2 | AAAGTTCCAGGCAACATCGT | 440 |
| GPX1 | NM_000581 | S8298/GPX1.p2 | CTCATCACCTGGTCTCCGGTGTGT | 441 |
| GRB7 | NM_005310 | S0130/GRB7.f2 | CCATCTGCATCCATCTTGTT | 442 |
| GRB7 | NM_005310 | S0132/GRB7.r2 | GGCCACCAGGGTATTATCTG | 443 |
| GRB7 | NM_005310 | S4726/GRB7.p2 | CTCCCCACCCTTGAGAAGTGCCT | 444 |
| GSK3B | NM_002093 | T0408/GSK3B.f2 | GACAAGGACGGCAGCAAG | 445 |
| GSK3B | NM_002093 | T0409/GSK3B.r2 | TTGTGGCCTGTCTGGACC | 446 |
| GSK3B | NM_002093 | T0410/GSK3B.p2 | CCAGGAGTTGCCACCACTGTTGTC | 447 |
| GSR | NM_000637 | S8633/GSR.f1 | GTGATCCCAAGCCCACAATA | 448 |
| GSR | NM_000637 | S8634/GSR.r1 | TGTGGCGATCAGGATGTG | 449 |
| GSR | NM_000637 | S8635/GSR.p1 | TCAGTGGGAAAAAGTACACCGCCC | 450 |
| GSTM1 | NM_000561 | S2026/GSTM1.r1 | GGCCCAGCTTGAATTTTTCA | 451 |
| GSTM1 | NM_000561 | S2027/GSTM1.f1 | AAGCTATGAGGAAAAGAAGTACACGAT | 452 |
| GSTM1 | NM_000561 | S4739/GSTM1.p1 | TCAGCCACTGGCTTCTGTCATAATCAGGAG | 453 |
| GSTp | NM_000852 | S0136/GSTp.f3 | GAGACCCTGCTGTCCCAGAA | 454 |
| GSTp | NM_000852 | S0138/GSTp.r3 | GGTTGTAGTCAGCGAAGGAGATC | 455 |
| GSTp | NM_000852 | S5007/GSTp.p3 | TCCCACAATGAAGGTCTTGCCTCCCT | 456 |
| GUS | NM_000181 | S0139/GUS.f1 | CCCACTCAGTAGCCAAGTCA | 457 |
| GUS | NM_000181 | S0141/GUS.r1 | CACGCAGGTGGTATCAGTCT | 458 |
| GUS | NM_000181 | S4740/GUS.p1 | TCAAGTAAACGGGCTGTTTTCCAAACA | 459 |
| HDAC6 | NM_006044 | S9451/HDAC6.f1 | TCCTGTGCTCTGGAAGCC | 460 |
| HDAC6 | NM_006044 | S9452/HDAC6.r1 | CTCCACGGTCTCAGTTGATCT | 461 |
| HDAC6 | NM_006044 | S9453/HDAC6.p1 | CAAGAACCTCCCAGAAGGGCTCAA | 462 |
| HER2 | NM_004448 | S0142/HER2.f3 | CGGTGTGAGAAGTGCAGCAA | 463 |
| HER2 | NM_004448 | S0144/HER2.r3 | CCTCTCGCAAGTGCTCCAT | 464 |
| HER2 | NM_004448 | S4729/HER2.p3 | CCAGACCATAGCACACTCGGGCAC | 465 |
| HIF1A | NM_001530 | S1207/HIF1A.f3 | TGAACATAAAGTCTGCAACATGGA | 466 |
| HIF1A | NM_001530 | S1208/HIF1A.r3 | TGAGGTTGGTTACTGTTGGTATCATATA | 467 |
| HIF1A | NM_001530 | S4753/HIF1A.p3 | TTGCACTGCACAGGCCACATTCAC | 468 |
| HNF3A | NM_004496 | S0148/HNF3A.f1 | TCCAGGATGTTAGGAACTGTGAAG | 469 |
| HNF3A | NM_004496 | S0150/HNF3A.r1 | GCGTGTCTGCGTAGTAGCTGTT | 470 |
| HNF3A | NM_004496 | S5008/HNF3A.p1 | AGTCGCTGGTTTCATGCCCTTCCA | 471 |
| HRAS | NM_005343 | S8427/HRAS.f1 | GGACGAATACGACCCCACT | 472 |
| HRAS | NM_005343 | S8428/HRAS.r1 | GCACGTCTCCCCATCAAT | 473 |
| HRAS | NM_005343 | S8429/HRAS.p1 | ACCACCTGCTTCCGGTAGGAATCC | 474 |
| HSPA1A | NM_005345 | S6708/HSPA1A.f1 | CTGCTGCGACAGTCCACTA | 475 |
| HSPA1A | NM_005345 | S6709/HSPA1A.r1 | CAGGTTCGCTCTGGGAAG | 476 |
| HSPA1A | NM_005345 | S6710/HSPA1A.p1 | AGAGTGACTCCCGTTGTCCCAAGG | 477 |
| HSPA1B | NM_005346 | S6714/HSPA1B.f1 | GGTCCGCTTCGTCTTTCGA | 478 |
| HSPA1B | NM_005346 | S6715/HSPA1B.r1 | GCACAGGTTCGCTCTGGAA | 479 |
| HSPA1B | NM_005346 | S6716/HSPA1B.p1 | TGACTCCCGCGGTCCCAAGG | 480 |
| HSPA1L | NM_005527 | T2015/HSPA1L.f1 | GCAGGTGTGATTGCTGGAC | 481 |
| HSPA1L | NM_005527 | T2016/HSPA1L.r1 | ACCATAGGCAATGGCAGC | 482 |
| HSPA1L | NM_005527 | T2017/HSPA1L.p1 | AAGAATCATCAATGAGCCCACGGC | 483 |
| HSPA5 | NM_005347 | S7166/HSPA5.f1 | GGCTAGTAGAACTGGATCCCAACA | 484 |
| HSPA5 | NM_005347 | S7167/HSPA5.r1 | GGTCTGCCCAAATGCTTTTC | 485 |
| HSPA5 | NM_005347 | S7168/HSPA5.p1 | TAATTAGACCTAGGCCTCAGCTGCACTGCC | 486 |
| HSPA9B | NM_004134 | T2018/HSPA9B.f1 | GGCCACTAAAGATGCTGGC | 487 |
| HSPA9B | NM_004134 | T2019/HSPA9B.r1 | AGCAGCTGTGGGCTCATT | 488 |
| HSPA9B | NM_004134 | T2020/HSPA9B.p1 | ATCACCCGAAGCACATTCAGTCCA | 489 |
| HSPB1 | NM_001540 | S6720/HSPB1.f1 | CCGACTGGAGGAGCATAAA | 490 |
| HSPB1 | NM_001540 | S6721/HSPB1.r1 | ATGCTGGCTGACTCTGCTC | 491 |
| HSPB1 | NM_001540 | S6722/HSPB1.p1 | CGCACTTTTCTGAGCAGACGTCCA | 492 |
| HSPCA | NM_005348 | S7097/HSPCA.f1 | CAAAAGGCAGAGGCTGATAA | 493 |
| HSPCA | NM_005348 | S7098/HSPCA.r1 | AGCGCAGTTTCATAAAGCAA | 494 |
| HSPCA | NM_005348 | S7099/HSPCA.p1 | TGACCAGATCCTTCACAGACTTGTCGT | 495 |
| ID1 | NM_002165 | S0820/ID1.f1 | AGAACCGCAAGGTGAGCAA | 496 |
| ID1 | NM_002165 | S0821/ID1.r1 | TCCAACTGAAGGTCCCTGATG | 497 |
| ID1 | NM_002165 | S4832/ID1.p1 | TGGAGATTCTCCAGCACGTCATCGAC | 498 |
| IFITM1 | NM_002165 | S7768/IFITM1.f1 | CACGCAGAAAACCACACTTC | 499 |
| IFITM1 | NM_002165 | S7769/IFITM1.r1 | CATGTTCCTCCTTGTGCATC | 500 |
| IFITM1 | NM_002165 | S7770/IFITM1.p1 | CAACACTTCCTTCCCCAAAGCCAG | 501 |
| IGF1R | NM_000875 | S1249/IGF1R.f3 | GCATGGTAGCCGAAGATTTCA | 502 |
| IGF1R | NM_000875 | S1250/IGF1R.r3 | TTTCCGGTAATAGTCTGTCTCATAGATATC | 503 |
| IGF1R | NM_000875 | S4895/IGF1R.p3 | CGCGTCATACCAAAATCTCCGATTTTGA | 504 |
| IGFBP2 | NM_000597 | S1128/IGFBP2.f1 | GTGGACAGCACCATGAACA | 505 |
| IGFBP2 | NM_000597 | S1129/IGFBP2.r1 | CCTTCATACCCGACTTGAGG | 506 |
| IGFBP2 | NM_000597 | S4837/IGFBP2.p1 | CTTCCGGCCAGCACTGCCTC | 507 |
| IGFBP3 | NM_000598 | S0157/IGFBP3.f3 | ACGCACCGGGTGTCTGA | 508 |
| IGFBP3 | NM_000598 | S0159/IGFBP3.r3 | TGCCCTTTCTTGATGATGATTATC | 509 |
| IGFBP3 | NM_000598 | S5011/IGFBP3.p3 | CCCAAGTTCCACCCCCTCCATTCA | 510 |
| IGFBP5 | NM_000599 | S1644/IGFBP5.f1 | TGGACAAGTACGGGATGAAGCT | 511 |
| IGFBP5 | NM_000599 | S1645/IGFBP5.r1 | CGAAGGTGTGGCACTGAAAGT | 512 |
| IGFBP5 | NM_000599 | S4908/IGFBP5.p1 | CCCGTCAACGTACTCCATGCCTGG | 513 |
| IL-7 | NM_000880 | S5781/IL-7.f1 | GCGGTGATTCGGAAATTCG | 514 |
| IL-7 | NM_000880 | S5782/IL-7.r1 | CTCTCCTGGGCACCTGCTT | 515 |
| IL-7 | NM_000880 | S5783/IL-7.p1 | CTCTGGTCCTCATCCAGGTGCGC | 516 |
| IL-8 | NM_000584 | S5790/IL-8.f1 | AAGGAACCATCTCACTGTGTGTAAAC | 517 |
| IL-8 | NM_000584 | S5791/IL-8.r1 | ATCAGGAAGGCTGCCAAGAG | 518 |
| IL-8 | NM_000584 | S5792/IL-8.p1 | TGACTTCCAAGCTGGCCGTGGC | 519 |
| IL2RA | NM_000417 | T2147/IL2RA.f1 | TCTGCGTGGTTCCTTTCTCA | 520 |
| IL2RA | NM_000417 | T2148/IL2RA.r1 | TTGAAGGATGTTTATTAGGCAACGT | 521 |
| IL2RA | NM_000417 | T2149/IL2RA.p1 | CGCTTCTGACTGCTGATTCTCCCGTT | 522 |
| IL6 | NM_000600 | S0760/IL6.f3 | CCTGAACCTTCCAAAGATGG | 523 |
| IL6 | NM_000600 | S0761/IL6.r3 | ACCAGGCAAGTCTCCTCATT | 524 |
| IL6 | NM_000600 | S4800/IL6.p3 | CCAGATTGGAAGCATCCATCTTTTTCA | 525 |
| IL8RB | NM_001557 | T2168/IL8RB.f1 | CCGCTCCGTCACTGATGTCT | 526 |
| IL8RB | NM_001557 | T2169/IL8RB.r1 | GCAAGGTCAGGGCAAAGAGTA | 527 |
| IL8RB | NM_001557 | T2170/IL8RB.p1 | CCTGCTGAACCTAGCCTTGGCCGA | 528 |
| ILK | NM_001014794 | T0618/ILK.f1 | CTCAGGATTTTCTCGCATCC | 529 |
| ILK | NM_001014794 | T0619/ILK.r1 | AGGAGCAGGTGGAGACTGG | 530 |
| ILK | NM_001014794 | T0618/ILK.p1 | ATGTGCTCCCAGTGCTAGGTGCCT | 531 |
| ILT-2 | NM_006669 | S1611/ILT-2.f2 | AGCCATCACTCTCAGTGCAG | 532 |
| ILT-2 | NM_006669 | S1612/ILT-2.r2 | ACTGCAGAGTCAGGGTCTCC | 533 |
| ILT-2 | NM_006669 | S4904/ILT-2.p2 | CAGGTCCTATCGTGGCCCCTGA | 534 |
| INCENP | NM_020238 | T2024/INCENP.f1 | GCCAGGATACTGGAGTCCATC | 535 |
| INCENP | NM_020238 | T2025/INCENP.r1 | CTTGACCCTTGGGGTCCT | 536 |
| INCENP | NM_020238 | T2026/INCENP.p1 | TGAGCTCCCTGATGGCTACACCC | 537 |
| IRAK2 | NM_001570 | T2027/IRAK2.f1 | GGATGGAGTTCGCCTCCT | 538 |
| IRAK2 | NM_001570 | T2028/IRAK2.r1 | CGCTCCATGGACTTGATCTT | 539 |
| IRAK2 | NM_001570 | T2029/IRAK2.p1 | CGTGATCACAGACCTGACCCAGCT | 540 |
| IRS1 | NM_005544 | S1943/IRS1.f3 | CCACAGCTCACCTTCTGTCA | 541 |
| IRS1 | NM_005544 | S1944/IRS1.r3 | CCTCAGTGCCAGTCTCTTCC | 542 |
| IRS1 | NM_005544 | S5050/IRS1.p3 | TCCATCCCAGCTCCAGCCAG | 543 |
| ITGB1 | NM_002211 | S7497/ITGB1.f1 | TCAGAATTGGATTTGGCTCA | 544 |
| ITGB1 | NM_002211 | S7498/ITGB1.r1 | CCTGAGCTTAGCTGGTGTTG | 545 |
| ITGB1 | NM_002211 | S7499/ITGB1.p1 | TGCTAATGTAAGGCATCACAGTCTTTTCCA | 546 |
| K-Alpha-1 | NM_006082 | S8706/K-Alph.f2 | TGAGGAAGAAGGAGAGGAATACTAAT | 547 |
| K-Alpha-1 | NM_006082 | S8707/K-Alph.r2 | CTGAAATTCTGGGAGCATGAC | 548 |
| K-Alpha-1 | NM_006082 | S8708/K-Alph.p2 | TATCCATTCCTTTTGGCCCTGCAG | 549 |
| KDR | NM_002253 | S1343/KDR.f6 | GAGGACGAAGGCCTCTACAC | 550 |
| KDR | NM_002253 | S1344/KDR.r6 | AAAAATGCCTCCACTTTTGC | 551 |
| KDR | NM_002253 | S4903/KDR.p6 | CAGGCATGCAGTGTTCTTGGCTGT | 552 |
| Ki-67 | NM_002417 | S0436/Ki-67.f2 | CGGACTTTGGGTGCGACTT | 553 |
| Ki-67 | NM_002417 | S0437/Ki-67.r2 | TTACAACTCTTCCACTGGGACGAT | 554 |
| Ki-67 | NM_002417 | S4741/Ki-67.p2 | CCACTTGTCGAACCACCGCTCGT | 555 |
| KIF11 | NM_004523 | T2409/KIF11.f2 | TGGAGGTTGTAAGCCAATGT | 556 |
| KIF11 | NM_004523 | T2410/KIF11.r2 | TGCCTTACGTCCATCTGATT | 557 |
| KIF11 | NM_004523 | T2411/KIF11.p2 | CAGTGATGTCTGAACTTGAAGCCTCACA | 558 |
| KIF22 | NM_007317 | S8505/KIF22.f1 | CTAAGGCACTTGCTGGAAGG | 559 |
| KIF22 | NM_007317 | S8506/KIF22.r1 | TCTTCCCAGCTCCTGTGG | 560 |
| KIF22 | NM_007317 | S8507/KIF22.p1 | TCCATAGGCAAGCACACTGGCATT | 561 |
| KIF2C | NM_006845 | S7262/KIF2C.f1 | AATTCCTGCTCCAAAAGAAAGTCTT | 562 |
| KIF2C | NM_006845 | S7263/KIF2C.r1 | CGTGATGCGAAGCTCTGAGA | 563 |
| KIF2C | NM_006845 | S7264/KIF2C.p1 | AAGCCGCTCCACTCGCATGTCC | 564 |
| KIFC1 | NM_002263 | S8517/KIFC1.f1 | CCACAGGGTTGAAGAACCAG | 565 |
| KIFC1 | NM_002263 | S8519/KIFC1.r1 | CACCTGATGTGCCAGACTTC | 566 |
| KIFC1 | NM_002263 | S8520/KIFC1.p1 | AGCCAGTTCCTGCTGTTCCTGTCC | 567 |
| KLK10 | NM_002776 | S2624/KLK10.f3 | GCCCAGAGGCTCCATCGT | 568 |
| KLK10 | NM_002776 | S2625/KLK10.r3 | CAGAGGTTTGAACAGTGCAGACA | 569 |
| KLK10 | NM_002776 | S4978/KLK10.p3 | CCTCTTCCTCCCCAGTCGGCTGA | 570 |
| KNS2 | NM_005552 | T2030/KNS2.f1 | CAAACAGAGGGTGGCAGAAG | 571 |
| KNS2 | NM_005552 | T2031/KNS2.r1 | GAGGCTCTCACGGCTCCT | 572 |
| KNS2 | NM_005552 | T2032/KNS2.p1 | CGCTTCTCCATGTTCTCAGGGTCA | 573 |
| KNTC1 | NM_014708 | T2126/KNTC1.f1 | AGCCGAGGCTTTGTTGAA | 574 |
| KNTC1 | NM_014708 | T2127/KNTC1.r1 | TGGGCTATGAGCACAGCTT | 575 |
| KNTC1 | NM_014708 | T2128/KNTC1.p1 | TTCATATCCAGTACCGGCGATCGG | 576 |
| KNTC2 | NM_006101 | S7296/KNTC2.f1 | ATGTGCCAGTGAGCTTGAGT | 577 |
| KNTC2 | NM_006101 | S7297/KNTC2.r1 | TGAGCCCCTGGTTAACAGTA | 578 |
| KNTC2 | NM_006101 | S7298/KNTC2.p1 | CCTTGGAGAAACACAAGCACCTGC | 579 |
| KRT14 | NM_000526 | S1853/KRT14.f1 | GGCCTGCTGAGATCAAAGAC | 580 |
| KRT14 | NM_000526 | S1854/KRT14.r1 | GTCCACTGTGGCTGTGAGAA | 581 |
| KRT14 | NM_000526 | S5037/KRT14.p1 | TGTTCCTCAGGTCCTCAATGGTCTTG | 582 |
| KRT17 | NM_000422 | S0172/KRT17.f2 | CGAGGATTGGTTCTTCAGCAA | 583 |
| KRT17 | NM_000422 | S0173/KRT17.p2 | CACCTCGCGGTTCAGTTCCTCTGT | 584 |
| KRT17 | NM_000422 | S0174/KRT17.r2 | ACTCTGCACCAGCTCACTGTTG | 585 |
| KRT19 | NM_002276 | S1515/KRT19.f3 | TGAGCGGCAGAATCAGGAGTA | 586 |
| KRT19 | NM_002276 | S1516/KRT19.r3 | TGCGGTAGGTGGCAATCTC | 587 |
| KRT19 | NM_002276 | S4866/KRT19.p3 | CTCATGGACATCAAGTCGCGGCTG | 588 |
| KRT5 | NM_000424 | S0175/KRT5.f3 | TCAGTGGAGAAGGAGTTGGA | 589 |
| KRT5 | NM_000424 | S0177/KRT5.r3 | TGCCATATCCAGAGGAAACA | 590 |
| KRT5 | NM_000424 | S5015/KRT5.p3 | CCAGTCAACATCTCTGTTGTCACAAGCA | 591 |
| L1CAM | NM_000425 | T1341/L1CAM.f1 | CTTGCTGGCCAATGCCTA | 592 |
| L1CAM | NM_000425 | T1342/L1CAM.r1 | TGATTGTCCGCAGTCAGG | 593 |
| L1CAM | NM_000425 | T1343/L1CAM.p1 | ATCTACGTTGTCCAGCTGCCAGCC | 594 |
| LAMC2 | NM_005562 | S2826/LAMC2.f2 | ACTCAAGCGGAAATTGAAGCA | 595 |
| LAMC2 | NM_005562 | S2827/LAMC2.r2 | ACTCCCTGAAGCCGAGACACT | 596 |
| LAMC2 | NM_005562 | S4969/LAMC2.p2 | AGGTCTTATCAGCACAGTCTCCGCCTCC | 597 |
| LAPTM4B | NM_018407 | T2063/LAPTM4.f1 | AGCGATGAAGATGGTCGC | 598 |
| LAPTM4B | NM_018407 | T2064/LAPTM4.r1 | GACATGGCAGCACAAGCA | 599 |
| LAPTM4B | NM_018407 | T2065/LAPTM4.p1 | CTGGACGCGGTTCTACTCCAACAG | 600 |
| LIMK1 | NM_016735 | T0759/LIMK1.f1 | GCTTCAGGTGTTGTGACTGC | 601 |
| LIMK1 | NM_016735 | T0760/LIMK1.r1 | AAGAGCTGCCCATCCTTCTC | 602 |
| LIMK1 | NM_016735 | T0761/LIMK1.p1 | TGCCTCCCTGTCGCACCAGTACTA | 603 |
| LIMK2 | NM_005569 | T2033/LIMK2.f1 | CTTTGGGCCAGGAGGAAT | 604 |
| LIMK2 | NM_005569 | T2034/LIMK2.r1 | CTCCCACAATCCACTGCC | 605 |
| LIMK2 | NM_005569 | T2035/LIMK2.p1 | ACTCGAATCCACCCAGGAACTCCC | 606 |
| MAD1L1 | NM_003550 | S7299/MAD1L1.f1 | AGAAGCTGTCCCTGCAAGAG | 607 |
| MAD1L1 | NM_003550 | S7300/MAD1L1.r1 | AGCCGTACCAGCTCAGACTT | 608 |
| MAD1L1 | NM_003550 | S7301/MAD1L1.p1 | CATGTTCTTCACAATCGCTGCATCC | 609 |
| MAD2L1 | NM_002358 | S7302/MAD2L1.f1 | CCGGGAGCAGGGAATCAC | 610 |
| MAD2L1 | NM_002358 | S7303/MAD2L1.r1 | ATGCTGTTGATGCCGAATGA | 611 |
| MAD2L1 | NM_002358 | S7304/MAD2L1.p1 | CGGCCACGATTTCGGCGCT | 612 |
| MAD2L1BP | NM_014628 | T2123/MAD2L1.f1 | CTGTCATGTGGCAGACCTTC | 613 |
| MAD2L1BP | NM_014628 | T2124/MAD2L1.r1 | TAAATGTCACTGGTGCCTGG | 614 |
| MAD2L1BP | NM_014628 | T2125/MAD2L1.p1 | CGAACCACGGCTTGGGAAGACTAC | 615 |
| MAD2L2 | NM_006341 | T1125/MAD2L2.f1 | GCCCAGTGGAGAAATTCGT | 616 |
| MAD2L2 | NM_006341 | T1126/MAD2L2.r1 | GCGAGTCTGACTGATGGA | 617 |
| MAD2L2 | NM_006341 | T1127/MAD2L2.p1 | TTTGAGATCACCCAGCCTCCACTG | 618 |
| MAGE2 | NM_005361 | S5623/MAGE2.f1 | CCTCAGAAATTGCCAGGACT | 619 |
| MAGE2 | NM_005361 | S5625/MAGE2.p1 | TTCCCGTGATCTTCAGCAAAGCCT | 620 |
| MAGE2 | NM_005361 | S5626/MAGE2.r1 | CCAAAGACCAGCTGCAAGTA | 621 |
| MAGE6 | NM_005363 | S5639/MAGE6.f3 | AGGACTCCAGCAACCAAGAA | 622 |
| MAGE6 | NM_005363 | S5640/MAGE6.r3 | GAGTGCTGCTTGGAACTCAG | 623 |
| MAGE6 | NM_005363 | S5641/MAGE6.p3 | CAAGCACCTTCCCTGACCTGGAGT | 624 |
| MAP2 | NM_031846 | S8493/MAP2.f1 | CGGACCACCAGGTCAGAG | 625 |
| MAP2 | NM_031846 | S8494/MAP2.r1 | CAGGGGTAGTGGGTGTTGAG | 626 |
| MAP2 | NM_031846 | S8495/MAP2.p1 | CCACTCTTCCCTGCTCTGCGAATT | 627 |
| MAP2K3 | NM_002756 | T2090/MAP2K3.f1 | GCCCTCCAATGTCCTTATCA | 628 |
| MAP2K3 | NM_002756 | T2091/MAP2K3.r1 | GTAGCCACTGATGCCAAAGTC | 629 |
| MAP2K3 | NM_002756 | T2092/MAP2K3.p1 | CACATCTTCACATGGCCCTCCTTG | 630 |
| MAP4 | NM_002375 | S5724/MAP4.f1 | GCCGGTCAGGCACACAAG | 631 |
| MAP4 | NM_002375 | S5725/MAP4.r1 | GCAGCATACACACAACAAAATGG | 632 |
| MAP4 | NM_002375 | S5726/MAP4.p1 | ACCAACCAGTCCACGCTCCAAGGG | 633 |
| MAP6 | NM_033063 | T2341/MAP6.f2 | CCCTCAACCGGCAAATCC | 634 |
| MAP6 | NM_033063 | T2342/MAP6.r2 | CGTCCATGCCCTGAATTCA | 635 |
| MAP6 | NM_033063 | T2343/MAP6.p2 | TGGCGAGTGCAGTGAGCAGCTCC | 636 |
| MAPK14 | NM_139012 | S5557/MAPK14.f2 | TGAGTGGAAAAGCCTGACCTATG | 637 |
| MAPK14 | NM_139012 | S5558/MAPK14.r2 | GGACTCCATCTCTTCTTGGTCAA | 683 |
| MAPK14 | NM_139012 | S5559/MAPK14.p2 | TGAAGTCATCAGCTTTGTGCCACCACC | 639 |
| MAPK8 | NM_002750 | T2087/MAPK8.f1 | CAACACCCGTACATCAATGTCT | 640 |
| MAPK8 | NM_002750 | T2088MAPK8.r1 | TCATCTAACTGCTTGTCAGGGA | 641 |
| MAPK8 | NM_002750 | T2089/MAPK8.p1 | CTGAAGCAGAAGCTCCACCACCAA | 642 |
| MAPRE1 | NM_012325 | T2180/MAPRE1.f1 | GACCTTGGAACCTTTGGAAC | 643 |
| MAPRE1 | NM_012325 | T2181/MAPRE1.r1 | CCTAGGCCTATGAGGGTTCA | 644 |
| MAPRE1 | NM_012325 | T2182/MAPRE1.p1 | CAGCCCTGTAAGACCTGTTGACAGCA | 645 |
| MAPT | NM_016835 | S8502/MAPT.f1 | CACAAGCTGACCTTCCGC | 646 |
| MAPT | NM_016835 | S8503/MAPT.r1 | ACTTGTACACGATCTCCGCC | 647 |
| MAPT | NM_016835 | S8504/MAPT.p1 | AGAACGCCAAAGCCAAGACAGACC | 648 |
| Maspin | NM_002639 | S0836/Maspin.f2 | CAGATGGCCACTTTGAGAACATT | 649 |
| Maspin | NM_002639 | S0837/Maspin.r2 | GGCAGCATTAACCACAAGGATT | 650 |
| Maspin | NM_002639 | S4835/Maspin.p2 | AGCTGACAACAGTGTGAACGACCAGACC | 651 |
| MCL1 | NM_021960 | S5545/MCL1.f1 | CTTCGGAAACTGGACATCAA | 652 |
| MCL1 | NM_021960 | S5546/MCL1.r1 | GTCGCTGAAAACATGGATCA | 653 |
| MCL1 | NM_021960 | S5547/MCL1.p1 | TCACTCGAGACAACGATTTCACATCG | 654 |
| MCM2 | NM_004526 | S1602/MCM2.f2 | GACTTTTGCCCGCTACCTTTC | 655 |
| MCM2 | NM_004526 | S1603/MCM2.r2 | GCCACTAACTGCTTCAGTATGAAGAG | 656 |
| MCM2 | NM_004526 | S4900/MCM2.p2 | ACAGCTCATTGTTGTCACGCCGGA | 657 |
| MCM6 | NM_005915 | S1704/MCM6.f3 | TGATGGTCCTATGTGTCACATTCA | 658 |
| MCM6 | NM_005915 | S1705/MCM6.r3 | TGGGACAGGAAACACACCAA | 659 |
| MCM6 | NM_005915 | S4919/MCM6.p3 | CAGGTTTCATACCAACACAGGCTTCAGCAC | 660 |
| MCP1 | NM_002982 | S1955/MCP1.f1 | CGCTCAGCCAGATGCAATC | 661 |
| MCP1 | NM_002982 | S1956/MCP1.r1 | GCACTGAGATCTTCCTATTGGTGAA | 662 |
| MCP1 | NM_002982 | S5052/MCP1.p1 | TGCCCCAGTCACCTGCTGTTA | 663 |
| MGMT | NM_002412 | S1922/MGMT.f1 | GTGAAATGAAACGCACCACA | 664 |
| MGMT | NM_002412 | S1923/MGMT.r1 | GACCCTGCTCACAACCAGAC | 665 |
| MGMT | NM_002412 | S5045/MGMT.p1 | CAGCCCTTTGGGGAAGCTGG | 666 |
| MMP12 | NM_002426 | S4381/MMP12.f2 | CCAACGCTTGCCAAATCCT | 667 |
| MMP12 | NM_002426 | S4382/MMP12.r2 | ACGGTAGTGACAGCATCAAAACTC | 668 |
| MMP12 | NM_002426 | S4383/MMP12.p2 | AACCAGCTCTCTGTGACCCCAATT | 669 |
| MMP2 | NM_004530 | S1874/MMP2.f2 | CCATGATGGAGAGGCAGACA | 670 |
| MMP2 | NM_004530 | S1875/MMP2.r2 | GGAGTCCGTCCTTACCGTCAA | 671 |
| MMP2 | NM_004530 | S5039/MMP2.p2 | CTGGGAGCATGGCGATGGATACCC | 672 |
| MMP9 | NM_004994 | S0656/MMP9.f1 | GAGAACCAATCTCACCGACA | 673 |
| MMP9 | NM_004994 | S0657/MMP9.r1 | CACCCGAGTGTAACCATAGC | 674 |
| MMP9 | NM_004994 | S4760/MMP9.p1 | ACAGGTATTCCTCTGCCAGCTGCC | 675 |
| MRE11A | NM_005590 | T2039/MRE11A.f1 | GCCATGCTGGCTCAGTCT | 676 |
| MRE11A | NM_005590 | T2040/MRE11A.r1 | CACCCAGACCCACCTAACTG | 677 |
| MRE11A | NM_005590 | T2041/MRE11A.p1 | CACTAGCTGATGTGGCCCACAGCT | 678 |
| MRP1 | NM_004996 | S0181/MRP1.f1 | TCATGGTGCCCGTCAATG | 679 |
| MRP1 | NM_004996 | S0183/MRP1.r1 | CGATTGTCTTTGCTCTTCATGTG | 680 |
| MRP1 | NM_004996 | S5019/MRP1.p1 | ACCTGATACGTCTTGGTCTTCATCGCCAT | 681 |
| MRP2 | NM_000392 | S0184/MRP2.f3 | AGGGGATGACTTGGACACAT | 682 |
| MRP2 | NM_000392 | S0186/MRP2.r3 | AAAACTGCATGGCTTTGTCA | 683 |
| MRP2 | NM_000392 | S5021/MRP2.p3 | CTGCCATTCGACATGACTGCAATTT | 684 |
| MRP3 | NM_003786 | S0187/MRP3.f1 | TCATCCTGGCGATCTACTTCCT | 685 |
| MRP3 | NM_003786 | S0189/MRP3.r1 | CCGTTGAGTGGAATCAGCAA | 686 |
| MRP3 | NM_003786 | S5023/MRP3.p1 | TCTGTCCTGGCTGGAGTCGCTTTCAT | 687 |
| MSH3 | NM_002439 | S5940/MSH3.f2 | TGATTACCATCATGGCTCAGA | 688 |
| MSH3 | NM_002439 | S5941/MSH3.r2 | CTTGTGAAAATGCCATCCAC | 689 |
| MSH3 | NM_002439 | S5942/MSH3.p2 | TCCCAATTGTCGCTTCTTCTGCAG | 690 |
| MUC1 | NM_002456 | S0782/MUC1.f2 | GGCCAGGATCTGTGGTGGTA | 691 |
| MUC1 | NM_002456 | S0783/MUC1.r2 | CTCCACGTCGTGGACATTGA | 692 |
| MUC1 | NM_002456 | S4807/MUC1.p2 | CTCTGGCCTTCCGAGAAGGTACC | 693 |
| MX1 | NM_002462 | S7611/MX1.f1 | GAAGGAATGGGAATCAGTCATGA | 694 |
| MX1 | NM_002462 | S7612/MX1.r1 | GTCTATTAGAGTCAGATCCGGGACAT | 695 |
| MX1 | NM_002462 | S7613/MX1.p1 | TCACCCTGGAGATCAGCTCCCGA | 696 |
| MYBL2 | NM_002466 | S3270/MYBL2.f1 | GCCGAGATCGCCAAGATG | 697 |
| MYBL2 | NM_002466 | S3271/MYBL2.r1 | CTTTTGATGGTAGAGTTCCAGTGATTC | 698 |
| MYBL2 | NM_002466 | S4742/MYBL2.p1 | CAGCATTGTCTGTCCTCCCTGGCA | 699 |
| MYH11 | NM_002474 | S4555/MYH11.f1 | CGGTACTTCTCAGGGCTAATATATACG | 700 |
| MYH11 | NM_002474 | S4556/MYH11.r1 | CCGAGTAGATGGGCAGGTGTT | 701 |
| MYH11 | NM_002474 | S4557/MYH11.p1 | CTCTTCTGCGTGGTGGTCAACCCCTA | 702 |
| NEK2 | NM_002497 | S4327/NEK2.f1 | GTGAGGCAGCGCGACTCT | 703 |
| NEK2 | NM_002497 | S4328/NEK2.r1 | TGCCAATGGTGTACAACACTTCA | 704 |
| NEK2 | NM_002497 | S4329/NEK2.p1 | TGCCTTCCCGGGCTGAGGACT | 705 |
| NFKBp50 | NM_003998 | S9961/NFKBp5.f3 | CAGACCAAGGAGATGGACCT | 706 |
| NFKBp50 | NM_003998 | S9962/NFKBp5.r3 | AGCTGCCAGTGCTATCCG | 707 |
| NFKBp50 | NM_003998 | S9963/NFKBp5.p3 | AAGCTGTAAACATGAGCCGCACCA | 708 |
| NFKBp65 | NM_021975 | S0196/NFKBp6.f3 | CTGCCGGGATGGCTTCTAT | 709 |
| NFKBp65 | NM_021975 | S0198/NFKBp6.r3 | CCAGGTTCTGGAAACTGTGGAT | 710 |
| NFKBp65 | NM_021975 | S5030/NFKBp6.p3 | CTGAGCTCTGCCCGGACCGCT | 711 |
| NME6 | NM_005793 | T2129/NME6.f1 | CACTGACACCCGCAACAC | 712 |
| NME6 | NM_005793 | T2130/NME6.r1 | GGCTGCAATCTCTCTGCTG | 713 |
| NME6 | NM_005793 | T2131/NME6.p1 | AACCACAGAGTCCGAACCATGGGT | 714 |
| NPC2 | NM_006432 | T2141/NPC2.f1 | CTGCTTCTTTCCCGAGCTT | 715 |
| NPC2 | NM_006432 | T2142/NPC2.r1 | AGCAGGAATGTAGCTGCCA | 716 |
| NPC2 | NM_006432 | T2143/NPC2.p1 | ACTTCGTTATCCGCGATGCGTTTC | 717 |
| NPD009 | NM_020686 | S4474/NPD009.f3 | GGCTGTGGCTGAGGCTGTAG | 718 |
| (ABAT official) | | | | |
| NPD009 | NM_020686 | S4475/NPD009.r3 | GGAGCATTCGAGGTCAAATCA | 719 |
| (ABAT official) | | | | |
| NPD009 | NM_020686 | S4476/NPD009.p3 | TTCCCAGAGTGTCTCACCTCCAGCAGAG | 720 |
| (ABAT official) | | | | |
| NTSR2 | NM_012344 | T2332/NTSR2.f2 | CGGACCTGAATGTAATGCAA | 721 |
| NTSR2 | NM_012344 | T2333/NTSR2.r2 | CTTTGCCAGGTGACTAAGCA | 722 |
| NTSR2 | NM_012344 | T2334/NTSR2.p2 | AATGAACAGAACAAGCAAAATGACCAGC | 723 |
| NUSAP1 | NM_016359 | S7106/NUSAP1.f1 | CAAAGGAAGAGCAACGGAAG | 724 |
| NUSAP1 | NM_016359 | S7107/NUSAP1.r1 | ATTCCCAAAACCTTTGCTT | 725 |
| NUSAP1 | NM_016359 | S7108/NUSAP1.p1 | TTCTCCTTTCGTTCTTGCTCGCGT | 726 |
| p21 | NM_000389 | S0202/p21.f3 | TGGAGACTCTCAGGGTCGAAA | 727 |
| p21 | NM_000389 | S0204/p21.r3 | GGCGTTTGGAGTGGTAGAAATC | 728 |
| p21 | NM_000389 | S5047/p21.p3 | CGGCGGCAGACCAGCATGAC | 729 |
| p27 | NM_004064 | S0205/p27.f3 | CGGTGGACCACGAAGAGTTAA | 730 |
| p27 | NM_004064 | S0207/p27.r3 | GGCTCGCCTCTTCCATGTC | 731 |
| p27 | NM_004064 | S4750/p27.p3 | CCGGGACTTGGAGAAGCACTGCA | 732 |
| PCTK1 | NM_006201 | T2075/PCTK1.f1 | TCACTACCAGCTGACATCCG | 733 |
| PCTK1 | NM_006201 | T2076/PCTK1.r1 | AGATGGGGCTATTGAGGGTC | 734 |
| PCTK1 | NM_006201 | T2077/PCTK1.p1 | CTTCTCCAGGTAGCCCTCAGGCAG | 735 |
| PDGFRb | NM_002609 | S1346/PDGFRb.f3 | CCAGCTCTCCTTCCAGCTAC | 736 |
| PDGFRb | NM_002609 | S1347/PDGFRb.r3 | GGGTGGCTCTCACTTAGCTC | 737 |
| PDGFRb | NM_002609 | S4931/PDGFRb.p3 | ATCAATGTCCCTGTCCGAGTGCTG | 738 |
| PFDN5 | NM_145897 | T2078/PFDN5.f1 | GAGAAGCACGCCATGAAAC | 739 |
| PFDN5 | NM_145897 | T2079/PFDN5.r1 | GGCTGTGAGCTGCTGAATCT | 740 |
| PFDN5 | NM_145897 | T2080/PFDN5.p1 | TGACTCATCATTTCCATGACGGCC | 741 |
| PGK1 | NM_000291 | S0232/PGK1.f1 | AGAGCCAGTTGCTGTAGAACTCAA | 742 |
| PGK1 | NM_000291 | S0234/PGK1.r1 | CTGGGCCTACACAGTCCTTCA | 743 |
| PGK1 | NM_000291 | S5022/PGK1.p1 | TCTCTGCTGGGCAAGGATGTTCTGTTC | 744 |
| PHB | NM_002634 | T2171/PHB.f1 | GACATTGTGGTAGGGGAAGG | 745 |
| PHB | NM_002634 | T2172/PHB.r1 | CGGCAGTCAAAGATAATTGG | 746 |
| PHB | NM_002634 | T2173/PHB.p1 | TCATTTTCTCATCCCGTGGGTACAGA | 747 |
| PI3KC2A | NM_002645 | S2020/PI3KC2.r1 | CACACTAGCATTTTCTCCGCATA | 748 |
| PI3KC2A | NM_002645 | S2021/PI3KC2.f1 | ATACCAATCACCGCACAAACC | 749 |
| PI3KC2A | NM_002645 | S5062/PI3KC2.p1 | TGCGCTGTGACTGGACTTAACAAATAGCCT | 750 |
| PIM1 | NM_002648 | S7858/PIM1.f3 | CTGCTCAAGGACACCGTCTA | 751 |
| PIM1 | NM_002648 | S7859/PIM1.r3 | GGATCCACTCTGGAGGGC | 752 |
| PIM1 | NM_002648 | S7860/PIM1.p3 | TACACTCGGGTCCCATCGAAGTCC | 753 |
| PIM2 | NM_006875 | T2144/PIM2.f1 | TGGGGACATTCCCTTTGAG | 754 |
| PIM2 | NM_006875 | T2145/PIM2.r1 | GACATGGGCTGGGAAGTG | 755 |
| PIM2 | NM_006875 | T2146/PIM2.p1 | CAGCTTCCAGAATCTCCTGGTCCC | 756 |
| PLAUR | NM_002659 | S1976/PLAUR.f3 | CCCATGGATGCTCCTCTGAA | 757 |
| PLAUR | NM_002659 | S1977/PLAUR.r3 | CCGGTGGCTACCAGACATTG | 758 |
| PLAUR | NM_002659 | S5054/PLAUR.p3 | CATTGACTGCCGAGGCCCCATG | 759 |
| PLD3 | NM_012268 | S8645/PLD3.f1 | CCAAGTTCTGGGTGGTGG | 760 |
| PLD3 | NM_012268 | S8646/PLD3.r1 | GTGAACGCCAGTCCATGTT | 761 |
| PLD3 | NM_012268 | S8647/PLD3.p1 | CCAGACCCACTTCTACCTGGGCAG | 762 |
| PLK | NM_005030 | S3099/PLK.f3 | AATGAATACAGTATTCCCAAGCACAT | 763 |
| PLK | NM_005030 | S3100/PLK.r3 | TGTCTGAAGCATCTTCTGGATGA | 764 |
| PLK | NM_005030 | S4825/PLK.p3 | AACCCCGTGGCCGCCTCC | 765 |
| PMS1 | NM_000534 | S5894/PMS1.f2 | CTTACGGTTTTCGTGGAGAAG | 766 |
| PMS1 | NM_000534 | S5895/PMS1.r2 | AGCAGCCGTTCTTGTTGTAA | 767 |
| PMS1 | NM_000534 | S5896/PMS1.p2 | CCTCAGCTATACAACAAATTGACCCCAAG | 768 |
| PMS2 | NM_000535 | S5878/PMS2.f3 | GATGTGGACTGCCATTCAAA | 769 |
| PMS2 | NM_000535 | S5879/PMS2.r3 | TGCGAGATTAGTTGGCTGAG | 770 |
| PMS2 | NM_000535 | S5880/PMS2.p3 | TCGAAATTTACATCCGGTATCTTCCTGG | 771 |
| PP591 | NM_025207 | S8657/PP591.f1 | CCACATACCGTCCAGCCTA | 772 |
| PP591 | NM_025207 | S8658/PP591.r1 | GAGGTCATGTGCGGGAGT | 773 |
| PP591 | NM_025207 | S8659/PP591.p1 | CCGCTCCTCTTCTTCGTTCTCCAG | 774 |
| PPP2CA | NM_002715 | T0732/PPP2CA.f1 | GCAATCATGGAACTTGACGA | 775 |
| PPP2CA | NM_002715 | T0733/PPP2CA.r1 | ATGTGGCTCGCCTCTACG | 776 |
| PPP2CA | NM_002715 | T0734/PPP2CA.p1 | TTTCTTGCAGTTTGACCCAGCACC | 777 |
| PR | NM_000926 | S1336/PR.f6 | GCATCAGGCTGTCATTATGG | 778 |
| PR | NM_000926 | S1337/PR.r6 | AGTAGTTGTGCTGCCCTTCC | 779 |
| PR | NM_000926 | S4743/PR.p6 | TGTCCTTACCTGTGGGAGCTGTAAGGTC | 780 |
| PRDX1 | NM_002574 | T1241/PRDX1.f1 | AGGACTGGGACCCATGAAC | 781 |
| PRDX1 | NM_002574 | T1242/PRDX1.r1 | CCCATAATCCTGAGCAATGG | 782 |
| PRDX1 | NM_002574 | T1243/PRDX1.p1 | TCCTTTGGTATCAGACCCGAAGCG | 783 |
| PRDX2 | NM_005809 | S8761/PRDX2.f1 | GGTGTCCTTCGCCAGATCAC | 784 |
| PRDX2 | NM_005809 | S8762/PRDX2.r1 | CAGCCGCAGAGCCTCATC | 785 |
| PRDX2 | NM_005809 | S8763/PRDX2.p1 | TTAATGATTTGCCTGTGGGACGCTCC | 786 |
| PRKCA | NM_002737 | S7369/PRKCA.f1 | CAAGCAATGCGTCATCAATGT | 787 |
| PRKCA | NM_002737 | S7370/PRKCA.r1 | GTAAATCCGCCCCCTCTTCT | 788 |
| PRKCA | NM_002737 | S7371/PRKCA.p1 | CAGCCTCTGCGGAATGGATCACACT | 789 |
| PRKCD | NM_006254 | S1738/PRKCD.f2 | CTGACACTTGCCGCAGAGAA | 790 |
| PRKCD | NM_006254 | S1739/PRKCD.r2 | AGGTGGTCCTTGGTCTGGAA | 791 |
| PRKCD | NM_006254 | S4923/PRKCD.p2 | CCCTTTCTCACCCACCTCATCTGCAC | 792 |
| PRKCG | NM_002739 | T2081/PRKCG.f1 | GGGTTCTAGACGCCCCTC | 793 |
| PRKCG | NM_002739 | T2082/PRKCG.r1 | GGACGGCTGTAGAGGCTGTAT | 794 |
| PRKCG | NM_002739 | T2083/PRKCG.p1 | CAAGCGTTCCTGGCCTTCTGAACT | 795 |
| PRKCG | NM_006255 | T2084/PRKCH.f1 | CTCCACCTATGAGCGTCTGTC | 796 |
| PRKCG | NM_006255 | T2085/PRKCH.r1 | CACACTTTCCCTCCTTTTGG | 797 |
| PRKCG | NM_006255 | T2086/PRKCH.p1 | TCCTGTTAACATCCCAAGCCCACA | 798 |
| pS2 | NM_003225 | S0241/pS2.f2 | GCCCTCCCAGTGTGCAAAT | 799 |
| pS2 | NM_003225 | S0243/pS2.r2 | CGTCGATGGTATTAGGATAGAAGCA | 800 |
| pS2 | NM_003225 | S5026/pS2.p2 | TGCTGTTTCGACGACACCGTTCG | 801 |
| PTEN | NM_000315 | S0244/PTEN.f2 | TGGCTAAGTGAAGATGACAATCATG | 802 |
| PTEN | NM_000315 | S0246/PTEN.r2 | TGCACATATCATTACACCAGTTCGT | 803 |
| PTEN | NM_000315 | S5027/PTEN.p2 | CCTTTCCAGCTTTACAGTGAATTGCTGCA | 804 |
| PTPD1 | NM_007039 | S3069/PTPD1.f2 | CGCTTGCCTAACTCATACTTTCC | 805 |
| PTPD1 | NM_007039 | S3070/PTPD1.r2 | CCATTCAGACTGCGCCACTT | 806 |
| PTPD1 | NM_007039 | S4822/PTPD1.p2 | TCCACGCAGCGTGGCACTG | 807 |
| PTTG1 | NM_004219 | S4525/PTTG1.f2 | GGCTACTCTGATCTATGTTGATAAGGAA | 808 |
| PTTG1 | NM_004219 | S4526/PTTG1.r2 | GCTTCAGCCCATCCTTAGCA | 809 |
| PTTG1 | NM_004219 | S4527/PTTG1.p2 | CACACGGGTGCCTGGTTCTCCA | 810 |
| RAB27B | NM_004163 | S4336/RAB27B.f1 | GGGACACTGCGGGACAAG | 811 |
| RAB27B | NM_004163 | S4337/RAB27B.r1 | GCCCATGGCGTCTCTGAA | 812 |
| RAB27B | NM_004163 | S4338/RAB27B.p1 | CGGTTCCGGAGTCTCACCACTGCAT | 813 |
| RAB31 | NM_006868 | S9306/RAB31.f1 | CTGAAGGACCCTACGCTCG | 814 |
| RAB31 | NM_006868 | S9307/RAB31.r1 | ATGCAAAGCCAGTGTGCTC | 815 |
| RAB31 | NM_006868 | S9308/RAB31.p1 | CTTCTCAAAGTGAGGTGCCAGGCC | 816 |
| RAB6C | NM_032144 | S5535/RAB6C.f1 | GCGACAGCTCCTCTAGTTCCA | 817 |
| RAB6C | NM_032144 | S5537/RAB6C.p1 | TTCCCGAAGTCTCCGCCCG | 818 |
| RAB6C | NM_032144 | S5538/RAB6C.r1 | GGAACACCAGCTTGAATTTCCT | 819 |
| RAD1 | NM_002853 | T2174/RAD1.f1 | GAGGAGTGGTGACAGTCTGC | 820 |
| RAD1 | NM_002853 | T2175/RAD1.r1 | GCTGCAGAAATCAAAGTCCA | 821 |
| RAD1 | NM_002853 | T2176/RAD1.p1 | TCAATACACAGGAACCTGAGGAGACCC | 822 |
| RAD54L | NM_003579 | S4369/RAD54L.f1 | AGCTAGCCTCAGTGACACACATG | 823 |
| RAD54L | NM_003579 | S4370/RAD54L.r1 | CCGGATCTGACGGCTGTT | 824 |
| RAD54L | NM_003579 | S4371/RAD54L.p1 | ACACAACGTCGGCAGTGCAACCTG | 825 |
| RAF1 | NM_002880 | S5933/RAF1.f3 | CGTCGTATGCGAGAGTCTGT | 826 |
| RAF1 | NM_002880 | S5934/RAF1.r3 | TGAAGGCGTGAGGTGTAGAA | 827 |
| RAF1 | NM_002880 | S5935/RAF1.p3 | TCCAGGATGCCTGTTAGTTCTCAGCA | 828 |
| RALBP1 | NM_006788 | S5853/RALBP1.f1 | GGTGTCAGATATAAATGTGCAAATGC | 829 |
| RALBP1 | NM_006788 | S5854/RALBP1.r1 | TTCGATATTGCCAGCAGCTATAAA | 830 |
| RALBP1 | NM_006788 | S5855/RALBP1.p1 | TGCTGTCCTGTCGGTCTCAGTACGTTCA | 831 |
| RAP1GDS1 | NM_021159 | S5306/RAP1GD.f2 | TGTGGATGCTGGATTGATTT | 832 |
| RAP1GDS1 | NM_021159 | S5307/RAP1GD.r2 | AAGCAGCACTTCCTGGTCTT | 833 |
| RAP1GDS1 | NM_021159 | S5308/RAP1GD.p2 | CCACTGGTGCAGCTGCTAAATAGCA | 834 |
| RASSF1 | NM_007182 | S2393/RASSF1.f3 | AGTGGGAGACACCTGACCTT | 835 |
| RASSF1 | NM_007182 | S2394/RASSF1.r3 | TGATCTGGGCATTGTACTCC | 836 |
| RASSF1 | NM_007182 | S4909/RASSF1.p3 | TTGATCTTCTGCTCAATCTCAGCTTGAGA | 837 |
| RB1 | NM_000321 | S2700/RB1.f1 | CGAAGCCCTTACAAGTTTCC | 838 |
| RB1 | NM_000321 | S2701/RB1.r1 | GGACTCTTCAGGGGTGAAAT | 839 |
| RB1 | NM_000321 | S4765/RB1.p1 | CCCTTACGGATTCCTGGAGGGAAC | 840 |
| RBM17 | NM_032905 | S2186/RBM17.f1 | CCCAGTGTACGAGGAACAAG | 841 |
| RBM17 | NM_032905 | S2187/RBM17.r1 | TTAGCGAGGAAGGAGTTGCT | 842 |
| RBM17 | NM_032905 | S2188/RBM17.p1 | ACAGACCGAGATCTCCAACCGGAC | 843 |
| RCC1 | NM_001269 | S8854/RCC1.f1 | GGGCTGGGTGAGAATGTG | 844 |
| RCC1 | NM_001269 | S8855/RCC1.r1 | CACAACATCCTCCGGAATG | 845 |
| RCC1 | NM_001269 | S8856/RCC1.p1 | ATACCAGGGCCGGCTTCTTCCTCT | 846 |
| REG1A | NM_002909 | T2093/REG1A.f1 | CCTACAAGTCCTGGGGCA | 847 |
| REG1A | NM_002909 | T2094/REG1A.r1 | TGAGGTCAGGCTCACACAGT | 848 |
| REG1A | NM_002909 | T2095/REG1A.p1 | TGGAGCCCCAAGCAGTGTTAATCC | 849 |
| RELB | NM_006509 | T2096/RELB.f1 | GCGAGGAGCTCTACTTGCTC | 850 |
| RELB | NM_006509 | T2097/RELB.r1 | GCCCTGCTGAACACCACT | 851 |
| RELB | NM_006509 | T2098/RELB.p1 | TGTCCTCTTTCTGCACCTTGTCGC | 852 |
| RhoB | NM_004040 | S8284/RhoB.f1 | AAGCATGAACAGGACTTGACC | 853 |
| RhoB | NM_004040 | S8285/RhoB.r1 | CCTCCCCAAGTCAGTTGC | 854 |
| RhoB | NM_004040 | S8286/RhoB.p1 | CTTTCCAACCCCTGGGGAAGACAT | 855 |
| rhoC | NM_175744 | S2162/rhoC.f1 | CCCGTTCGGTCTGAGGAA | 856 |
| rhoC | NM_175744 | S2163/rhoC.r1 | GAGCACTCAAGGTAGCCAAAGG | 857 |
| rhoC | NM_175744 | S5042/rhoC.p1 | TCCGGTTCGCCATGTCCCG | 858 |
| RIZ1 | NM_012231 | S1320/RIZ1.f2 | CCAGACGAGCGATTAGAAGC | 859 |
| RIZ1 | NM_012231 | S1321/RIZ1.r2 | TCCTCCTCTTCCTCCTCCTC | 860 |
| RIZ1 | NM_012231 | S4761/RIZ1.p2 | TGTGAGGTGAATGATTTGGGGGA | 861 |
| ROCK1 | NM_005406 | S8305/ROCK1.f1 | TGTGCACATAGGAATGAGCTTC | 862 |
| ROCK1 | NM_005406 | S8306/ROCK1.r1 | GTTTAGCACGCAATTGCTCA | 863 |
| ROCK1 | NM_005406 | S8307/ROCK1.p1 | TCACTCTCTTTGCTGGCCAACTGC | 864 |
| RPL37A | NM_000998 | T2418/RPL37A.f2 | GATCTGGCACTGTGGTTCC | 865 |
| RPL37A | NM_000998 | T2419/RPL37A.r2 | TGACAGCGGAAGTGGTATTG | 866 |
| RPL37A | NM_000998 | T2420/RPL37A.p2 | CACCGCCAGCCACTGTCTTCAT | 867 |
| RPLPO | NM_001002 | S0256/RPLPO.f2 | CCATTCTATCATCAACGGGTACAA | 868 |
| RPLPO | NM_001002 | S0258/RPLPO.r2 | TCAGCAAGTGGGAAGGTGTAATC | 869 |
| RPLPO | NM_001002 | S4744/RPLPO.p2 | TCTCCACAGACAAGGCCAGGACTCG | 870 |
| RPN2 | NM_002951 | T1158/RPN2.f1 | CTGTCTTCCTGTTGGCCCT | 871 |
| RPN2 | NM_002951 | T1159/RPN2.r1 | GTGAGGTAGTGAGTGGGCGT | 872 |
| RPN2 | NM_002951 | T1160/RPN2.p1 | ACAATCATAGCCAGCACCTGGGCT | 873 |
| RPS6KB1 | NM_003161 | S2615/RPS6KB.f3 | GCTCATTATGAAAAACATCCCAAAC | 874 |
| RPS6KB1 | NM_003161 | S2616/RPS6KB.r3 | AAGAAACAGAAGTTGTCTGGCTTTCT | 875 |
| RPS6KB1 | NM_003161 | S4759/RPS6KB.p3 | CACACCAACCAATAATTTCGCATT | 876 |
| RXRA | NM_002957 | S8463/RXRA.f1 | GCTCTGTTGTGTCCTGTTGC | 877 |
| RXRA | NM_002957 | S8464/RXRA.r1 | GTACGGAGAAGCCACTTCACA | 878 |
| RXRA | NM_002957 | S8465/RXRA.p1 | TCAGTCACAGGAAGGCCAGAGCC | 879 |
| RXRB | NM_021976 | S8490/RXRB.f1 | CGAGGAGATGCCTGTGGA | 880 |
| RXRB | NM_021976 | S8491/RXRB.r1 | CAACGCCCTGGTCACTCT | 881 |
| RXRB | NM_021976 | S8492/RXRB.p1 | CTGTTCCACAGCAAGCTCTGCCTC | 882 |
| S100A10 | NM_002966 | S9950/S100A1.f1 | ACACCAAAATGCCATCTCAA | 883 |
| S100A10 | NM_002966 | S9951/S100A1.r1 | TTTATCCCCAGCGAATTTGT | 884 |
| S100A10 | NM_002966 | S9952/S100A1.p1 | CACGCCATGGAAACCATGATGTTT | 885 |
| SEC61A | NM_013336 | S8648/SEC61A.f1 | CTTCTGAGCCCGTCTCCC | 886 |
| SEC61A | NM_013336 | S8649/SEC61A.r1 | GAGAGCTCCCCTTCCGAG | 887 |
| SEC61A | NM_013336 | S8650/SEC61A.p1 | CGCTTCTGGAGCAGCTTCCTCAAC | 888 |
| SEMA3F | NM_004186 | S2857/SEMA3F.f3 | CGCGAGCCCCTCATTATACA | 889 |
| SEMA3F | NM_004186 | S2858/SEMA3F.r3 | CACTCGCCGTTGACATCCT | 890 |
| SEMA3F | NM_004186 | S4972/SEMA3F.p3 | CTCCCCACAGCGCATCGAGGAA | 891 |
| SFN | NM_006142 | S9953/SFN.f1 | GAGAGAGCCAGTCTGATCCA | 892 |
| SFN | NM_006142 | S9954/SFN.r1 | AGGCTGCCATGTCCTCATA | 893 |
| SFN | NM_006142 | S9955/SFN.p1 | CTGCTCTGCCAGCTTGGCCTTC | 894 |
| SGCB | NM_000232 | S5752/SGCB.f1 | CAGTGGAGACCAGTTGGGTAGTG | 895 |
| SGCB | NM_000232 | S5753/SGCB.r1 | CCTTGAAGAGCGTCCCATCA | 896 |
| SGCB | NM_000232 | S5754/SGCB.p1 | CACACATGCAGAGCTTGTAGCGTACCCA | 897 |
| SGK | NM_005627 | S8308/SGK.f1 | TCCGCAAGACACCTCCTG | 898 |
| SGK | NM_005627 | S8309/SGK.r1 | TGAAGTCATCCTTGGCCC | 899 |
| SGK | NM_005627 | S8310/SGK.p1 | TGTCCTGTCCTTCTGCAGGAGGC | 900 |
| SGKL | NM_170709 | T2183/SGKL.f1 | TGCATTCGTTGGTTTCTCTT | 901 |
| SGKL | NM_170709 | T2184/SGKL.r1 | TTTCTGAATGGCAAACTGCT | 902 |
| SGKL | NM_170709 | T2185/SGKL.p1 | TGCACCTCCTTCAGAAGACTTATTTTTGTG | 903 |
| SHC1 | NM_003029 | S6456/SHC1.f1 | CCAACACCTTCTTGGCTTCT | 904 |
| SHC1 | NM_003029 | S6457/SHC1.r1 | CTGTTATCCCAACCCAAACC | 905 |
| SHC1 | NM_003029 | S6458/SHC1.p1 | CCTGTGTTCTTGCTGAGCACCCTC | 906 |
| SIR2 | NM_012238 | S1575/SIR2.f2 | AGCTGGGGTGTCTGTTTCAT | 907 |
| SIR2 | NM_012238 | S1576/SIR2.r2 | ACAGCAAGGCGAGCATAAAT | 908 |
| SIR2 | NM_012238 | S4885/SIR2.p2 | CCTGACTTCAGGTCAAGGGATGG | 909 |
| SLC1A3 | NM_004172 | S8469/SLC1A3.f1 | GTGGGGAGCCCATCATCT | 910 |
| SLC1A3 | NM_004172 | S8470/SLC1A3.r1 | CCAGTCCACACTGAGTGCAT | 911 |
| SLC1A3 | NM_004172 | S8471/SLC1A3.p1 | CCAAGCCATCACAGGCTCTGCATA | 912 |
| SLC25A4 | NM_213611 | T0278/SLC25A.f2 | TCTGCCAGTGCTGAATTCTT | 913 |
| SLC25A4 | NM_213611 | T0279/SLC25A.r2 | TTCGAACCTTAGCAGCTTCC | 914 |
| SLC25A4 | NM_213611 | T0280/SLC25A.p2 | TGCTGACATTGCCCTGGCTCCTAT | 915 |
| SLC35B1 | NM_005827 | S8642/SLC35B.f1 | CCCAACTCAGGTCCTTGGTA | 916 |
| SLC35B1 | NM_005827 | S8643/SLC35B.r1 | CAAGAGGGTCACCCCAAG | 917 |
| SLC35B1 | NM_005827 | S8644/SLC35B.p1 | ATCCTGCAAGCCAATCCCAGTCAT | 918 |
| SLC7A11 | NM_014331 | T2045/SLC7A1.f1 | AGATGCATACTTGGAAGCACAG | 919 |
| SLC7A11 | NM_014331 | T2046/SLC7A1.r1 | AACCTAGGACCAGGTAACCACA | 920 |
| SLC7A11 | NM_014331 | T2047/SLC7A1.p1 | CATATCACACTGGGAGGCAATGCA | 921 |
| SLC7A5 | NM_003486 | S9244/SLC7A5.f2 | GCGCAGAGGCCAGTTAAA | 922 |
| SLC7A5 | NM_003486 | S9245/SLC7A5.r2 | AGCTGAGCTGTGGGTTGC | 923 |
| SLC7A5 | NM_003486 | S9246/SLC7A5.p2 | AGATCACCTCCTCGAACCCACTCC | 924 |
| SNAI2 | NM_003068 | S7824/SNAI2.f1 | GGCTGGCCAAACATAAGCA | 925 |
| SNAI2 | NM_003068 | S7825/SNAI2.r1 | TCCTTGTCACAGTATTTACAGCTGAA | 926 |
| SNAI2 | NM_003068 | S7826/SNAI2.p1 | CTGCACTGCGATGCCCAGTCTAGAAAATC | 927 |
| SNCA | NM_007308 | T2320/SNCA.f1 | AGTGACAAATGTTGGAGGAGC | 928 |
| SNCA | NM_007308 | T2321/SNCA.r1 | CCCTCCACTGTCTTCTGGG | 929 |
| SNCA | NM_007308 | T2322/SNCA.p1 | TACTGCTGTCACACCCGTCACCAC | 930 |
| SNCG | NM_003087 | T1704/SNCG.f1 | ACCCACCATGGATGTCTTC | 931 |
| SNCG | NM_003087 | T1705/SNCG.r1 | CCTGCTTGGTCTTTTCCAC | 932 |
| SNCG | NM_003087 | T1706/SNCG.p1 | AAGAAGGGCTTCTCCATCGCCAAG | 933 |
| SOD1 | NM_000454 | S7683/SOD1.f1 | TGAAGAGAGGCATGTTGGAG | 934 |
| SOD1 | NM_000454 | S7684/SOD1.r1 | AATAGACACATCGGCCACAC | 935 |
| SOD1 | NM_000454 | S7685/SOD1.p1 | TTTGTCAGCAGTCACATTGCCCAA | 936 |
| SRI | NM_003130 | T2177/SRI.f1 | ATACAGCACCAATGGAAAGATCAC | 937 |
| SRI | NM_003130 | T2178/SRI.r1 | TGTCTGTAAGAGCCCTCAGTTTGA | 938 |
| SRI | NM_003130 | T2179/SRI.p1 | TTCGACGACTACATCGCCTGCTGC | 939 |
| STAT1 | NM_007315 | S1542/STAT1.f3 | GGGCTCAGCTTTCAGAAGTG | 940 |
| STAT1 | NM_007315 | S1543/STAT1.r3 | ACATGTTCAGCTGGTCCACA | 941 |
| STAT1 | NM_007315 | S4878/STAT1.p3 | TGGCAGTTTTCTTCTGTCACCAAAA | 942 |
| STAT3 | NM_003150 | S1545/STAT3.f1 | TCACATGCCACTTTGGTGTT | 943 |
| STAT3 | NM_003150 | S1546/STAT3.r1 | CTTGCAGGAAGCGGCTATAC | 944 |
| STAT3 | NM_003150 | S4881/STAT3.p1 | TCCTGGGAGAGATTGACCAGCA | 945 |
| STK10 | NM_005990 | T2099/STK10.f1 | CAAGAGGGACTCGGACTGC | 946 |
| STK10 | NM_005990 | T2100/STK10.r1 | CAGGTCAGTGGAGAGATTGGT | 947 |
| STK10 | NM_005990 | T2101/STK10.p1 | CCTCTGCACCTCTGAGAGCATGGA | 948 |
| STK11 | NM_000455 | S9454/STK11.f1 | GGACTCGGAGACGCTGTG | 949 |
| STK11 | NM_000455 | S9455/STK11.r1 | GGGATCCTTCGCAACTTCTT | 950 |
| STK11 | NM_000455 | S9456/STK11.p1 | TTCTTGAGGATCTTGACGGCCCTC | 951 |
| STK15 | NM_003600 | S0794/STK15.f2 | CATCTTCCAGGAGGACCACT | 952 |
| STK15 | NM_003600 | S0795/STK15.r2 | TCCGACCTTCAATCATTTCA | 953 |
| STK15 | NM_003600 | S4745/STK15.p2 | CTCTGTGGCACCCTGGACTACCTG | 954 |
| STMN1 | NM_005563 | S5838/STMN1.f1 | AATACCCAACGCACAAATGA | 955 |
| STMN1 | NM_005563 | S5839/STMN1.r1 | GGAGACAATGCAAACCACAC | 956 |
| STMN1 | NM_005563 | S5840/STMN1.p1 | CACGTTCTCTGCCCCGTTTCTTG | 957 |
| STMY3 | NM_005940 | S2067/STMY3.f3 | CCTGGAGGCTGCAACATACC | 958 |
| STMY3 | NM_005940 | S2068/STMY3.r3 | TACAATGGCTTTGGAGGATAGCA | 959 |
| STMY3 | NM_005940 | S4746/STMY3.p3 | ATCCTCCTGAAGCCCTTTTCGCAGC | 960 |
| SURV | NM_001168 | S0259/SURV.f2 | TGTTTTGATTCCCGGGCTTA | 961 |
| SURV | NM_001168 | S0261/SURV.r2 | CAAAGCTGTCAGCTCTAGCAAAAG | 962 |
| SURV | NM_001168 | S4747/SURV.p2 | TGCCTTCTTCCTCCCTCACTTCTCACCT | 963 |
| TACC3 | NM_006342 | S7124/TACC3.f1 | CACCCTTGGACTGGAAAACT | 964 |
| TACC3 | NM_006342 | S7125/TACC3.r1 | CCTTGATGAGCTGTTGGTTC | 965 |
| TACC3 | NM_006342 | S7126/TACC3.p1 | CACACCCGGTCTGGACACAGAAAG | 966 |
| TBCA | NM_004607 | T2284/TBCA.f1 | GATCCTCGCGTGAGACAGA | 967 |
| TBCA | NM_004607 | T2285/TBCA.r1 | CACTTTTTCTTTGACCAACCG | 968 |
| TBCA | NM_004607 | T2286/TBCA.p1 | TTCACCACGCCGGTCTTGATCTT | 969 |
| TBCC | NM_003192 | T2302/TBCC.f1 | CTGTTTTCCTGGAGGACTGC | 970 |
| TBCC | NM_003192 | T2303/TBCC.r1 | ACTGTGTATGCGGAGCTGTT | 971 |
| TBCC | NM_003192 | T2304/TBCC.p1 | CCACTGCCAGCACGCAGTCAC | 972 |
| TBCD | NM_005993 | T2287/TBCD.f1 | CAGCCAGGTGTACGAGACATT | 973 |
| TBCD | NM_005993 | T2288/TBCD.r1 | ACCTCGTCCAGCACATCC | 974 |
| TBCD | NM_005993 | T2289/TBCD.p1 | CTCACCTACAGTGACGTCGTGGGC | 975 |
| TBCE | NM_003193 | T2290/TBCE.f1 | TCCCGAGAGAGGAAAGCAT | 976 |
| TBCE | NM_003193 | T2291/TBCE.r1 | GTCGGGTGCCTGCATTTA | 977 |
| TBCE | NM_003193 | T2292/TBCE.p1 | ATACACAGTCCCTTCGTGGCTCCC | 978 |
| TBD | NM_016261 | S3347/TBD.f2 | CCTGGTTGAAGCCTGTTAATGC | 979 |
| TBD | NM_016261 | S3348/TBD.r2 | TGCAGACTTCTCATATTTGCTAAAGG | 980 |
| TBD | NM_016261 | S4864/TBD.p2 | CCGCTGGGTTTTCCACACGTTGA | 981 |
| TCP1 | NM_030752 | T2296/TCP1.f1 | CCAGTGTGTGTAACAGGGTCAC | 982 |
| TCP1 | NM_030752 | T2297/TCP1.r1 | TATAGCCTTGGGCCACCC | 983 |
| TCP1 | NM_030752 | T2298/TCP1.p1 | AGAATTCGACAGCCAGATGCTCCA | 984 |
| TFRC | NM_003234 | S1352/TFRC.f3 | GCCAACTGCTTTCATTTGTG | 985 |
| TFRC | NM_003234 | S1353/TFRC.r3 | ACTCAGGCCCATTTCCTTTA | 986 |
| TFRC | NM_003234 | S4748/TFRC.p3 | AGGGATCTGAACCAATACAGAGCAGACA | |
| THBS1 | NM_003246 | S6474/THBS1.f1 | CATCCGCAAAGTGACTGAAGAG | 988 |
| THBS1 | NM_003246 | S6475/THBS1.r1 | GTACTGAACTCCGTTGTGATAGCATAG | 989 |
| THBS1 | NM_003246 | S6476/THBS1.p1 | CCAATGAGCTGAGGCGGCCTCC | 990 |
| TK1 | NM_003258 | S0866/TK1.f2 | GCCGGGAAGACCGTAATTGT | 991 |
| TK1 | NM_003258 | S0927/TK1.r2 | CAGCGGCACCAGGTTCAG | 992 |
| TK1 | NM_003258 | S4798/TK1.p2 | CAAATGGCTTCCTCTGGAAGGTCCCA | 993 |
| TOP2A | NM_001067 | S0271/TOP2A.f4 | AATCCAAGGGGGAGAGTGAT | 994 |
| TOP2A | NM_001067 | S0273/TOP2A.r4 | GTACAGATTTTGCCCGAGGA | 995 |
| TOP2A | NM_001067 | S4777/TOP2A.p4 | CATATGGACTTTGACTCAGCTGTGGC | 996 |
| TOP3B | NM_003935 | T2114/TOP3B.f1 | GTGATGCCTTCCCTGTGG | 997 |
| TOP3B | NM_003935 | T2115/TOP3B.r1 | TCAGGTAGTCGGGTGGGTT | 998 |
| TOP3B | NM_003935 | T2116/TOP3B.p1 | TGCTTCTCCAGCATCTTCACCTCG | 999 |
| TP | NM_001953 | S0277/TP.f3 | CTATATGCAGCCAGAGATGTGACA | 1000 |
| TP | NM_001953 | S0279/TP.r3 | CCACGAGTTTCTTACTGAGAATGG | 1001 |
| TP | NM_001953 | S4779/TP.p3 | ACAGCCTGCCACTCATCACAGCC | 1002 |
| TP35BP1 | NM_005657 | S1747/TP53BP.f2 | TGCTGTTGCTGAGTCTGTTG | 1003 |
| TP35BP1 | NM_005657 | S1748/TP53BP.r2 | CTTGCCTGGCTTCACAGATA | 1004 |
| TP35BP1 | NM_005657 | S4924/TP53BP.p2 | CCAGTCCCCAGAAGACCATGTCTG | 1005 |
| TPT1 | NM_003295 | S9098/TPT1.f1 | GGTGTCGATATTGTCATGAACC | 1006 |
| TPT1 | NM_003295 | S9099/TPT1.r1 | GTAATCTTTGATGTACTTCTTGTAGGC | 1007 |
| TPT1 | NM_003295 | S9100/TPT1.p1 | TCACCTGCAGGAAACAAGTTTCACAAA | 1008 |
| TRAG3 | NM_004909 | S5881/TRAG3.f1 | GACGCTGGTCTGGTGAAGATG | 1009 |
| TRAG3 | NM_004909 | S5882/TRAG3.r1 | TGGGTGGTTGTTGGACAATG | 1010 |
| TRAG3 | NM_004909 | S5883/TRAG3.p1 | CCAGGAAACCACGAGCCTCCAGC | 1011 |
| TRAIL | NM_003810 | S2539/TRAIL.f1 | CTTCACAGTGCTCCTGCAGTCT | 1012 |
| TRAIL | NM_003810 | S2540/TRAIL.r1 | CATCTGCTTCAGCTCGTTGGT | 1013 |
| TRAIL | NM_003810 | S4980/TRAIL.p1 | AAGTACACGTAAGTTACAGCCACACA | 1014 |
| TS | NM_001071 | S0280/TS.f1 | GCCTCGGTGTGCCTTTCA | 1015 |
| TS | NM_001071 | S0282/TS.r1 | CGTGATGTGCGCAATCATG | 1016 |
| TS | NM_001071 | S4780/TS.p1 | CATCGCCAGCTACGCCCTGCTC | 1017 |
| TSPAN4 | NM_003271 | T2102/TSPAN4.f1 | CTGGTCAGCCTTCAGGGAC | 1018 |
| TSPAN4 | NM_003271 | T2103/TSPAN4.r1 | CTTCAGTTCTGGGCTGGC | 1019 |
| TSPAN4 | NM_003271 | T2104/TSPAN4.p1 | CTGAGCACCGCCTGGTCTCTTTC | 1020 |
| TTK | NM_003318 | NM_7247/TTK.f1 | TGCTTGTCAGTTGTCAACACCTT | 1021 |
| TTK | NM_003318 | NM_7248/TTK.r1 | TGGAGTGGCAAGTATTTGATGCT | 1022 |
| TTK | NM_003318 | NM_7249/TTK.p1 | TGGCCAACCTGCCTGTTTCCAGC | 1023 |
| TUBA1 | NM_006000 | S8578/TUBA1.f1 | TGTCACCCCGACTCAACGT | 1024 |
| TUBA1 | NM_006000 | S8579/TUBA1.r1 | ACGTGGACTGAGATGCATTCAC | 1025 |
| TUBA1 | NM_006000 | S8580/TUBA1.p1 | AGACGCACCGCCCGGACTCAC | 1026 |
| TUBA2 | NM_006001 | S8581/TUBA2.f1 | AGCTCAACATGCGTGAGTGT | 1027 |
| TUBA2 | NM_006001 | S8582/TUBA2.r1 | ATTGCCGATCTGGACTCCT | 1028 |
| TUBA2 | NM_006001 | S8583/TUBA2.p1 | ATCTCTATCCACGTGGGGCAGGC | 1029 |
| TUBA3 | NM_006009 | S8584/TUBA3.f1 | CTCTTACATCGACCGCCTAAGAG | 1030 |
| TUBA3 | NM_006009 | S8585/TUBA3.r1 | GCTGATGGCGGAGACGAA | 1031 |
| TUBA3 | NM_006009 | S8586/TUBA3.p1 | CGCGCTGTAAGAAGCAACAACCTCTCC | 1032 |
| TUBA4 | NM_025019 | T2415/TUBA4.f3 | GAGGAGGGTGAGTTCTCCAA | 1033 |
| TUBA4 | NM_025019 | T2416/TUBA4.r3 | ATGCCCACCTCCTTGTAATC | 1034 |
| TUBA4 | NM_025019 | T2417/TUBA4.p3 | CCATGAGGATATGACTGCCCTGGA | 1035 |
| TUBA6 | NM_032704 | S8590/TUBA6.f1 | GTCCCTTCGCCTCCTTCAC | 1036 |
| TUBA6 | NM_032704 | S8591/TUBA6.r1 | CGTGGATGGAGATGCACTCA | 1037 |
| TUBA6 | NM_032704 | S8592/TUBA6.p1 | CCGCAGACCCCTTCAAGTTCTAGTCATG | 1038 |
| TUBA8 | NM_018943 | T2412/TUBA8.f2 | CGCCCTACCTATACCAACCT | 1039 |
| TUBA8 | NM_018943 | T2413/TUBA8.r2 | CGGAGAGAAGCAGTGATTGA | 1040 |
| TUBA8 | NM_018943 | T2414/TUBA8.p2 | CAACCGCCTCATCAGTCAGATTGTG | 1041 |
| TUBB | NM_001069 | S5820/TUBB.f1 | CGAGGACGAGGCTTAAAAAC | 1042 |
| TUBB | NM_001069 | S5821/TUBB.r1 | ACCATGCTTGAGGACAACAG | 1043 |
| TUBB | NM_001069 | S5822/TUBB.p1 | TCTCAGATCAATCGTGCATCCTTAGTGAA | 1044 |
| TUBB classIII | NM_006086 | S8090/TUBB c.f3 | CGCCCTCCTGCAGTATTTATG | 1045 |
| TUBB classIII | NM_006086 | S8091/TUBB c.r3 | ACAGAGACAGGAGCAGCTCACA | 1046 |
| TUBB classIII | NM_006086 | S8092/TUBB c.p3 | CCTCGTCCTCCCCACCTAGGCCA | 1047 |
| TUBB1 | NM_030773 | S8093/TUBB1.f1 | ACACTGACTGGCATCCTGCTT | 1048 |
| TUBB1 | NM_030773 | S8094/TUBB1.r1 | GCTCTGTAGCTCCCCATGTACTAGT | 1049 |
| TUBB1 | NM_030773 | S8095/TUBB1.p1 | AGCCTCCAGAAGAGCCAGGTGCCT | 1050 |
| TUBB2 | NM_006088 | S8096/TUBB2.f1 | GTGGCCTAGAGCCTTCAGTC | 1051 |
| TUBB2 | NM_006088 | S8097/TUBB2.r1 | CAGGCTGGGAGTGAATAAAGA | 1052 |
| TUBB2 | NM_006088 | S8098/TUBB2.p1 | TTCACACTGCTTCCCTGCTTTCCC | 1053 |
| TUBB5 | NM_006087 | S8102/TUBB5.f1 | ACAGGCCCCATGCATCCT | 1054 |
| TUBB5 | NM_006087 | S8103/TUBB5.r1 | AGTTTCTCTCCCAGATAAGCTAAGG | 1055 |
| TUBB5 | NM_006087 | S8104/TUBB5.p1 | TGCCTCACTCCCCTCAGCCCC | 1056 |
| TUBBM | NM_032525 | S8105/TUBBM.f1 | CCCTATGGCCCTGAATGGT | 1057 |
| TUBBM | NM_032525 | S8106/TUBBM.r1 | ACTAATTACATGACTTGGCTGCATTT | 1058 |
| TUBBM | NM_032525 | S8107/TUBBM.p1 | TGAGGGGCCGACACCAACACAAT | 1059 |
| TUBBOK | NM_178014 | S8108/TUBBOK.f1 | AGTGGAATCCTTCCCTTTCC | 1060 |
| TUBBOK | NM_178014 | S8109/TUBBOK.r1 | CCCTTGATCCCTTTCTCTGA | 1061 |
| TUBBOK | NM_178014 | S8110/TUBBOK.p1 | CCTCACTCAGCTCCTTTCCCCTGA | 1062 |
| TUBBP | NM_178014 | S8111/TUBBP.f1 | GGAAGGAAAGAAGCATGGTCTACT | 1063 |
| TUBBP | NM_178014 | S8112/TUBBP.r1 | AAAAAGTGACAGGCAACAGTGAAG | 1064 |
| TUBBP | NM_178014 | S8113/TUBBP.p1 | CACCAGAGACCCAGCGCACACCTA | 1065 |
| TUBG1 | NM_001070 | T2299/TUBG1.f1 | GATGCCGAGGGAAATCATC | 1066 |
| TUBG1 | NM_001070 | T2300/TUBG1.r1 | CCAGAACTCGAACCCAATCT | 1067 |
| TUBG1 | NM_001070 | T2301/TUBG1.p1 | ATTGCCGCACTGGCCCAACTGTAG | 1068 |
| TWIST1 | NM_000474 | S7929/TWIST1.f1 | GCGCTGCGGAAGATCATC | 1069 |
| TWIST1 | NM_000474 | S7930/TWIST1.r1 | GCTTGAGGGTCTGAATCTTGCT | 1070 |
| TWIST1 | NM_000474 | S7931/TWIST1.p1 | CCACGCTGCCCTCGGACAAGC | 1071 |
| TYRO3 | NM_006293 | T2105/TYRO3.f1 | CAGTGTGGAGGGGATGGA | 1072 |
| TYRO3 | NM_006293 | T2106/TYRO3.r1 | CAAGTTCTGGACCACAGCC | 1073 |
| TYRO3 | NM_006293 | T2107/TYRO3.p1 | CTTCACCCACTGGATGTCAGGCTC | 1074 |
| UFM1 | NM_016617 | T1284/UFM1.f2 | AGTTGTCGTGTGTTCTGGATTCA | 1075 |
| UFM1 | NM_016617 | T1285/UFM1.r2 | CGTCAGCGTGATCTTAAAGGAA | 1076 |
| UFM1 | NM_016617 | T1286/UFM1.p2 | TCCGGCACCACCATGTCGAAGG | 1077 |
| upa | NM_002658 | S0283/upa.f3 | GTGGATGTGCCCTGAAGGA | 1078 |
| upa | NM_002658 | S0285/upa.r3 | CTGCGGATCCAGGGTAAGAA | 1079 |
| upa | NM_002658 | S4769/upa.p3 | AAGCCAGGCGTCTACACGAGAGTCTCAC | 1080 |
| V-RAF | NM_001654 | S5763/V-RAF.f1 | GGTTGTGCTCTACGAGCTTATGAC | 1081 |
| V-RAF | NM_001654 | S5764/V-RAF.r1 | CGGCCCACCATAAAGATAATCT | 1082 |
| V-RAF | NM_001654 | S5765/V-RAF.p1 | TGCCTTACAGCCACATTGGCTGCC | 1083 |
| VCAM1 | NM_001078 | S3505/VCAM1.f1 | TGGCTTCAGGAGCTGAATACC | 1084 |
| VCAM1 | NM_001078 | S3506/VCAM1.r1 | TGCTGTCGTGATGAGAAAATAGTG | 1085 |
| VCAM1 | NM_001078 | S3507/VCAM1.p1 | CAGGCACACACAGGTGGGACACAAAT | 1086 |
| VEGF | NM_003376 | S0286/VEGF.f1 | CTGCTGTCTTGGGTGCATTG | 1087 |
| VEGF | NM_003376 | S0288/VEGF.r1 | GCAGCCTGGGACCACTTG | 1088 |
| VEGF | NM_003376 | S4782/VEGF.p1 | TTGCCTTGCTGCTCTACCTCCACCA | 1089 |
| VEGFB | NM_003377 | S2724/VEGFB.f1 | TGACGATGGCCTGGAGTGT | 1090 |
| VEGFB | NM_003377 | S2725/VEGFB.r1 | GGTACCGGATCATGAGGATCTG | 1091 |
| VEGFB | NM_003377 | S4960/VEGFB.p1 | CTGGGCAGCACCAAGTCCGGA | 1092 |
| VEGFC | NM_005429 | S2251/VEGFC.f1 | CCTCAGCAAGACGTTATTTGAAATT | 1093 |
| VEGFC | NM_005429 | S2252/VEGFC.r1 | AAGTGTGATTGGCAAAACTGATTG | 1094 |
| VEGFC | NM_005429 | S4758/VEGFC.p1 | CCTCTCTCTCAAGGCCCCAAACCAGT | 1095 |
| VHL | NM_000551 | T1359/VHL.f1 | CGGTTGGTGACTTGTCTGC | 1096 |
| VHL | NM_000551 | T1360/VHL.r1 | AAGACTTGTCCCTGCCTCAC | 1097 |
| VHL | NM_000551 | T1361/VHL.p1 | ATGCCTCAGTCTTCCCAAAGCAGG | 1098 |
| VIM | NM_003380 | S0790/VIM.f3 | TGCCCTTAAAGGAACCAATGA | 1099 |
| VIM | NM_003380 | S0791/VIM.r3 | GCTTCAACGGCAAAGTTCTCTT | 1100 |
| VIM | NM_003380 | S4810/VIM.p3 | ATTTCACGCATCTGGCGTTCCA | 1101 |
| WAVE3 | NM_006646 | T2640/WAVE3.f1 | CTCTCCAGTGTGGGCACC | 1102 |
| WAVE3 | NM_006646 | T2641/WAVE3.r1 | GCGGTGTAGCTCCCAGAGT | 1103 |
| WAVE3 | NM_006646 | T2642/WAVE3.p1 | CCAGAACAGATGCGAGCAGTCCAT | 1104 |
| Wnt-5a | NM_003392 | S6183/Wnt-5a.f1 | GTATCAGGACCACATGCAGTACATC | 1105 |
| Wnt-5a | NM_003392 | S6184/Wnt-5a.r1 | TGTCGGAATTGATACTGGCATT | 1106 |
| Wnt-5a | NM_003392 | S6185/Wnt-5a.p1 | TTGATGCCTGTCTTCGCGCCTTCT | 1107 |
| XIAP | NM_001167 | S0289/XIAP.f1 | GCAGTTGGAAGACACAGGAAAGT | 1108 |
| XIAP | NM_001167 | S0291/XIAP.r1 | TGCGTGGCACTATTTTCAAGA | 1109 |
| XIAP | NM_001167 | S4752/XIAP.p1 | TCCCCAAATTGCAGATTTATCAACGGC | 1110 |
| XIST | M97168 | S1844/XIST.f1 | CAGGTCAGGCAGAGGAAGTC | 1111 |
| XIST | M97168 | S1845/XIST.r1 | CCTAACAAGCCCCAAATCAA | 1112 |
| XIST | M97168 | S8271/XIST.p1 | TGCATTGCATGAGCTAAACCTATCTGA | 1113 |
| ZW10 | NM_004724 | T2117/ZW10.f1 | TGGTCAGATGCTGCTGAAGT | 1114 |
| ZW10 | NM_004724 | T2118/ZW10.r1 | ATCACAGCATGAAGGGATGG | 1115 |
| ZW10 | NM_004724 | T2119/ZW10.p1 | TATCCTTAGGCCGCTGGCATCTTG | 1116 |
| ZWILCH | NM_017975 | T2057/ZWILCH.f1 | GAGGGAGCAGACAGTGGGT | 1117 |
| ZWILCH | NM_017975 | T2058/ZWILCH.r1 | TCAGAGCCCTTGCTAAGTCAC | 1118 |
| ZWILCH | NM_017975 | T2059/ZWILCH.p1 | CCACGATCTCCGTAACCATTTGCA | 1119 |
| ZWINT | NM_007057 | S8920/ZWINT.f1 | TAGAGGCCATCAAAATTGGC | 1120 |
| ZWINT | NM_007057 | S8921/ZWINT.r1 | TCCGTTTCCTCTGGGCTT | 1121 |
| ZWINT | NM_007057 | S8922/ZWINT.p1 | ACCAAGGCCCTGACTCAGATGGAG | 1122 |

APPENDIX 2

| | | | |
|---|---|---|---|
| Gene Name | Accession # | Amplicon Sequence | SEQ ID NO: |

| | | | |
|---|---|---|---|
| ABCA9 | NM_080283 | TTACCCGTGGGAACTGTCTCCAAATA | 1123 |
| | | CATACTTCCTCTCACCAGGACAACAA | |
| | | CCACAGGATCCTCTGACCCATTTACT | |
| | | GGTC | |
| ABCB1 | NM_000927 | AAACACCACTGGAGCATTGACTACCA | 1124 |
| | | GGCTCGCCAATGATGCTGCTCAAGTT | |
| | | AAAGGGGCTATAGGTTCCAGGCTTG | |
| ABCB5 | NM_178559 | AGACAGTCGCCTTGGTCGGTCTCAAT | 1125 |
| | | GGCAGTGGGAAGAGTACGGTAGTCCA | |
| | | GCTTCTGCAGAGGTT | |
| ABCC10 | NM_033450 | ACCAGTGCCACAATGCAGTGGCTGGA | 1126 |
| | | CATTCGGCTACAGCTCATGGGGGCGG | |
| | | CAGTGGTCAGCGCTAT | |
| ABCC11 | NM_032583 | AAGCCACAGCCTCCATTGACATGGAG | 1127 |
| | | ACAGACACCCTGATCCAGCGCACAAT | |
| | | CCGTGAAGCCTTCC | |
| ABCC5 | NM_005688 | TGCAGACTGTACCATGCTGACCATTG | 1128 |
| | | CCCATCGCCTGCACACGGTTCTAGGC | |
| | | TCCGATAGGATTATGGTGCTGGCC | |
| ABCD1 | NM_000033 | TCTGTGGCCCACCTCTACTCCAACCT | 1129 |
| | | GACCAAGCCACTCCTGGACGTGGCTG | |
| | | TGACTTCCTACACCC | |
| ACTG2 | NM_001615 | ATGTACGTCGCCATTCAAGCTGTGCT | 1130 |
| | | CTCCCTCTATGCCTCTGGCCGCACGA | |
| | | CAGGCATCGTCCTGGATTCAGGTGAT | |
| | | GGCGT | |
| ACTR2 | NM_005722 | ATCCGCATTGAAGACCCACCCCGCAG | 1131 |
| | | AAAGCACATGGTATTCCTGGGTGGTG | |
| | | CAGTTCTAGCGGAT | |
| ACTR3 | NM_005721 | CAACTGCTGAGAGACCGAGAAGTAGG | 1132 |
| | | AATCCCTCCAGAACAATCCTTGGAAA | |
| | | CTGCTAAGGCAGTAAAGGAGCG | |
| AK055699 | NM_194317 | CTGCATGTGATTGAATAAGAAACAAG | 1133 |
| | | AAAGTGACCACACCAAAGCCTCCCTG | |
| | | GCTGGTGTACAGGGATCAGGTCCACA | |
| AKT1 | NM_005163 | CGCTTCTATGGCGCTGAGATTGTGTC | 1134 |
| | | AGCCCTGGACTACCTGCACTCGGAGA | |
| | | AGAACGTGGTGTACCGGGA | |
| AKT2 | NM_001626 | TCCTGCCACCCTTCAAACCTCAGGTC | 1135 |
| | | ACGTCCGAGGTCGACACAAGGTACTT | |
| | | CGATGATGAATTTACCGCC | |
| AKT3 | NM_005465 | TTGTCTCTGCCTTGGACTATCTACAT | 1136 |
| | | TCCGGAAAGATTGTGTACCGTGATCT | |
| | | CAAGTTGGAGAATCTAATGCTGG | |
| ANXA4 | NM_001153 | TGGGAGGGATGAAGGAAATTATCTGG | 1137 |
| | | ACGATGCTCTCGTGAGACAGGATGCC | |
| | | CAGGACCTGTATGAG | |
| APC | NM_000038 | GGACAGCAGGAATGTGTTTCTCCATA | 1138 |
| | | CAGGTCACGGGGAGCCAATGGTTCAG | |
| | | AAACAAATCGAGTGGGT | |
| APEX-1 | NM_001641 | GATGAAGCCTTTCGCAAGTTCCTGAA | 1139 |
| | | GGGCCTGGCTTCCCGAAAGCCCCTTG | |
| | | TGCTGTGTGGAGACCT | |
| APOC1 | NM_001645 | GGAAACACACTGGAGGACAAGGCTCG | 1140 |
| | | GGAACTCATCAGCCGCATCAAACAGA | |
| | | GTGAACTTTCTGCCAAGATGCG | |
| APOD | NM_001647 | GTTTATGCCATCGGCACCGTACTGGA | 1141 |
| | | TCCTGGCCACCGACTATGAGAACTAT | |
| | | GCCCTCGTGTATTCC | |
| APOE | NM_000041 | GCCTCAAGAGCTGGTTCGAGCCCCTG | 1142 |
| | | GTGGAAGACATGCAGCGCCAGTGGGC | |
| | | CGGGCTGGTGGAGAAGGTGCAGG | |
| APRT | NM_000485 | GAGGTCCTGGAGTGCGTGAGCCTGGT | 1143 |
| | | GGAGCTGACCTCGCTTAAGGGCAGGG | |
| | | AGAAGCTGGCACCT | |
| ARHA | NM_001664 | GGTCCTCCGTCGGTTCTCTCATTAGT | 1144 |
| | | CCACGGTCTGGTCTTCAGCTACCCGC | |
| | | CTTCGTCTCCGAGTTTGCGAC | |
| AURKB | NM_004217 | AGCTGCAGAAGAGCTGCACATTTGAC | 1145 |
| | | GAGCAGCGAACAGCCACGATCATGGA | |
| | | GGAGTTGGCAGATGC | |
| B-actin | NM_001101 | CAGCAGATGTGGATCAGCAAGCAGGA | 1146 |
| | | GTATGACGAGTCCGGCCCCTCCATCG | |
| | | TCCACCGCAAATGC | |
| BAD | NM_032989 | GGGTCAGGTGCCTCGAGATCGGGCTT | 1147 |
| | | GGGCCCAGAGCATGTTCCAGATCCCA | |
| | | GAGTTTGAGCCGAGTGAGCAG | |
| BAG1 | NM_004323 | CGTTGTCAGCACTTGGAATACAAGAT | 1148 |
| | | GGTTGCCGGGTCATGTTAATTGGGAA | |
| | | AAAGAACAGTCCACAGGAAGAGGTTG | |
| | | AAC | |
| Bak | NM_001188 | CCATTCCCACCATTCTACCTGAGGCC | 1149 |
| | | AGGACGTCTGGGGTGTGGGGATTGGT | |
| | | GGGTCTATGTTCCC | |
| Bax | NM_004324 | CCGCCGTGGACACAGACTCCCCCCGA | 1150 |
| | | GAGGTCTTTTTCCGAGTGGCAGCTGA | |
| | | CATGTTTTCTGACGGCAA | |
| BBC3 | NM_014417 | CCTGGAGGGTCCTGTACAATCTCATC | 1151 |
| | | ATGGGACTCCTGCCCTTACCCAGGGG | |
| | | CCACAGAGCCCCCGAGATGGAGCCCA | |
| | | ATTAG | |
| B-Catenin | NM_001904 | GGCTCTTGTGCGTACTGTCCTTCGGG | 1152 |
| | | CTGGTGACAGGGAAGACATCACTGAG | |
| | | CCTGCCATCTGTGCTCTTCGTCATCT | |
| | | GA | |
| Bcl2 | NM_000633 | CAGATGGACCTAGTACCCACTGAGAT | 1153 |
| | | TTCCACGCCGAAGGACAGCGATGGGA | |
| | | AAAATGCCCTTAAATCATAGG | |
| BCL2L11 | NM_138621 | AATTACCAAGCAGCCGAAGACCACCC | 1154 |
| | | ACGAATGGTTATCTTACGACTGTTAC | |
| | | GTTACATTGTCCGCCTG | |
| BCL2L13 | NM_015367 | CAGCGACAACTCTGGACAAGTCAGTC | 1155 |
| | | CCCCAGAGTCTCCAACTGTGACCACT | |
| | | TCCTGGCAGTCTGAGAGC | |
| Bclx | NM_001191 | CTTTTGTGGAACTCTATGGGAACAAT | 1156 |
| | | GCAGCAGCCGAGAGCCGAAAGGGCCA | |
| | | GGAACGCTTCAACCGCTG | |
| BCRP | NM_004827 | TGTACTGGCGAAGAATATTTGGTAAA | 1157 |
| | | GCAGGGCATCGATCTCTCACCCTGGG | |
| | | GCTTGTGGAAGAATCACGTGGC | |
| BID | NM_001196 | GGACTGTGAGGTCAACAACGGTTCCA | 1158 |
| | | GCCTCAGGGATGAGTGCATCACAAAC | |
| | | CTACTGGTGTTTGGCTTCC | |
| BIN1 | NM_004305 | CCTGCAAAAGGGAACAAGAGCCCTTC | 1159 |
| | | GCCTCCAGATGGCTCCCCTGCCGCCA | |
| | | CCCCCGAGATCAGAGTCAACCACG | |
| BRCA1 | NM_007295 | TCAGGGGGCTAGAAATCTGTTGCTAT | 1160 |
| | | GGGCCCTTCACCAACATGCCCACAGA | |
| | | TCAACTGGAATGG | |
| BRCA2 | NM_000059 | AGTTCGTGCTTTGCAAGATGGTGCAG | 1161 |
| | | AGCTTTATGAAGCAGTGAAGAATGCA | |
| | | GCAGACCCAGCTTACCTT | |
| BUB1 | NM_004336 | CCGAGGTTAATCCAGCACGTATGGGG | 1162 |
| | | CCAAGTGTAGGCTCCCAGCAGGAACT | |
| | | GAGAGCGCCATGTCTT | |
| BUB1B | NM_001211 | TCAACAGAAGGCTGAACCACTAGAAA | 1163 |
| | | GACTACAGTCCCAGCACCGACAATTC | |
| | | CAAGCTCGAGTGTCTCGGCAAACTCT | |
| | | GTTG | |
| BUB3 | NM_004725 | CTGAAGCAGATGGTTCATCATTTCCT | 1164 |
| | | GGGCTGTTAAACAAAGCGAGGTTAAG | |
| | | GTTAGACTCTTGGGAATCAGC | |
| C14orf10 | NM_017917 | GTCAGCGTGGTAGCGGTATTCTCCGC | 1165 |
| | | GGCAGTGACAGTAATTGTTTTTGCCT | |
| | | CTTTAGCCAAGACTTCC | |
| C20_orf1 | NM_012112 | TCAGCTGTGAGCTGCGGATACCGCCC | 1166 |
| | | GGCAATGGGACCTGCTCTTAACCTCA | |
| | | AACCTAGGACCGT | |
| CA9 | NM_001216 | ATCCTAGCCCTGGTTTTTGGCCTCCT | 1167 |
| | | TTTTGCTGTCACCAGCGTCGCGTTCC | |
| | | TTGTGCAGATGAGAAGGCAG | |
| CALD1 | NM_004342 | CACTAAGGTTTGAGACAGTTCCAGAA | 1168 |
| | | AGAACCCAAGCTCAAGACGCAGGACG | |
| | | AGCTCAGTTGTAGAGGGCTAATTCGC | |
| CAPZA1 | NM_006135 | TCGTTGGAGATCAGAGTGGAAGTTCA | 1169 |
| | | CCATCACACCACCTACAGCCCAGGTG | |
| | | GTTGGCGTGCTTAA | |
| CAV1 | NM_001753 | GTGGCTCAACATTGTGTTCCCATTTC | 1170 |
| | | AGCTGATCAGTGGGCCTCCAAGGAGG | |
| | | GGCTGTAAAATGGAGGCCATTG | |
| CCNB1 | NM_031966 | TTCAGGTTGTTGCAGGAGACCATGTA | 1171 |
| | | CATGACTGTCTCCATTATTGATCGGT | |
| | | TCATGCAGAATAATTGTGTGCCCAAG | |
| | | AAGATG | |
| CCND1 | NM_053056 | GCATGTTCGTGGCCTCTAAGATGAAG | 1172 |
| | | GAGACCATCCCCCTGACGGCCGAGAA | |
| | | GCTGTGCATCTACACCG | |
| CCNE2 | NM_057749 | ATGCTGTGGCTCCTTCCTAACTGGGG | 1173 |
| | | CTTTCTTGACATGTAGGTTGCTTGGT | |
| | | AATAACCTTTTTGTATATCACAATTT | |
| | | GGGT | |
| CCT3 | NM_001008800 | ATCCAAGGCCATGACTGGTGTGGAAC | 1174 |
| | | AATGGCCATACAGGGCTGTTGCCCAG | |
| | | GCCCTAGAGGTCATTCC | |
| CD14 | NM_000591 | GTGTGCTAGCGTACTCCCGCCTCAAG | 1175 |
| | | GAACTGACGCTCGAGGACCTAAAGAT | |
| | | AACCGGCACCATGC | |
| CD31 | NM_000442 | TGTATTTCAAGACCTCTGTGCACTTA | 1176 |
| | | TTTATGAACCTGCCCTGCTCCCACAG | |
| | | AACACAGCAATTCCTCAGGCTAA | |
| CD3z | NM_000734 | AGATGAAGTGGAAGGCGCTTTTCACC | 1177 |
| | | GCGGCCATCCTGCAGGCACAGTTGCC | |
| | | GATTACAGAGGCA | |
| CD63 | NM_001780 | AGTGGGACTGATTGCCGTGGGTGTCG | 1178 |
| | | GGGCACAGCTTGTCCTGAGTCAGACC | |
| | | ATAATCCAGGGGGCTACCC | |
| CD68 | NM_001251 | TGGTTCCCAGCCCTGTGTCCACCTCC | 1179 |
| | | AAGCCCAGATTCAGATTCGAGTCATG | |
| | | TACACAACCCAGGGTGGAGGAG | |
| CDC2 | NM_001786 | GAGAGCGACGCGGTTGTTGTAGCTGC | 1180 |
| | | CGCTGCGGCCGCCGCGGAATAATAAG | |
| | | CCGGGATCTACCATAC | |
| CDC20 | NM_001255 | TGGATTGGAGTTCTGGGAATGTACTG | 1181 |
| | | GCCGTGGCACTGGACAACAGTGTGTA | |
| | | CCTGTGGAGTGCAAGC | |
| CDC25B | NM_021873 | AAACGAGCAGTTTGCCATCAGACGCT | 1182 |
| | | TCCAGTCTATGCCGGTGAGGCTGCTG | |
| | | GGCCACAGCCCCGTGCTTCGGAACAT | |
| | | CACCAAC | |
| CDCA8 | NM_018101 | GAGGCACAGTATTGCCCAGCTGGATC | 1183 |
| | | CAGAGGCCTTGGGAAACATTAAGAAG | |
| | | CTCTCCAACCGTCTC | |
| CDH1 | NM_004360 | TGAGTGTCCCCCGGTATCTTCCCCGC | 1184 |
| | | CCTGCCAATCCCGATGAAATTGGAAA | |
| | | TTTTATTGATGAAAATCTGAAAGCGG | |
| | | CTG | |
| CDK5 | NM_004935 | AAGCCCTATCCGATGTACCCGGCCAC | 1185 |
| | | AACATCCCTGGTGAACGTCGTGCCCA | |
| | | AACTCAATGCCACAG | |
| CDKN1C | NM_000076 | CGGCGATCAAGAAGCTGTCCGGGCCT | 1186 |
| | | CTGATCTCCGATTTCTTCGCCAAGCG | |
| | | CAAGAGATCAGCGCCTG | |
| CEGP1 | NM_020974 | TGACAATCAGCACACCTGCATTCACC | 1187 |
| | | GCTCGGAAGAGGGCCTGAGCTGCATG | |
| | | AATAAGGATCACGGCTGTAGTCACA | |
| CENPA | NM_001809 | TAAATTCACTCGTGGTGTGGACTTCA | 1188 |
| | | ATTGGCAAGCCCAGGCCCTATTGGCC | |
| | | CTACAAGAGGC | |
| CENPE | NM_001813 | GGATGCTGGTGACCTCTTCTTCCCTC | 1189 |
| | | ACGTTGCAACAGGAATTAAAGGCTAA | |
| | | AAGAAAACGAAGAGTTACTTGGTGCC | |
| | | TTGGC | |
| CENPF | NM_016343 | CTCCCGTCAACAGCGTTCTTTCCAAA | 1190 |
| | | CACTGGACCAGGAGTGCATCCAGATG | |
| | | AAGGCCAGACTCACCC | |
| CGA | NM_001275 | CTGAAGGAGCTCCAAGACCTCGCTCT | 1191 |
| (CHGA official) | | CCAAGGCGCCAAGGAGAGGGCACATC | |
| | | AGCAGAAGAAACACAGCGGTTTTG | |
| CHFR | NM_018223 | AAGGAAGTGGTCCCTCTGTGGCAAGT | 1192 |
| | | GATGAAGTCTCCAGCTTTGCCTCAGC | |
| | | TCTCCCAGACAGAAAGACTGCGTC | |
| Chk1 | NM_001274 | GATAAATTGGTACAAGGGATCAGCTT | 1193 |
| | | TTCCCAGCCCACATGTCCTGATCATA | |
| | | TGCTTTTGAATAGTCAGTTACTTGGC | |
| | | ACCC | |
| Chk2 | NM_007194 | ATGTGGAACCCCCACCTACTTGGCGC | 1194 |
| | | CTGAAGTTCTTGTTTCTGTTGGGACT | |
| | | GCTGGGTATAACCGTGCTGTGGACTG | |
| cIAP2 | NM_001165 | GGATATTTCCGTGGCTCTTATTCAAA | 1195 |
| | | CTCTCCATCAAATCCTGTAAACTCCA | |
| | | GAGCAAATCAAGATTTTTCTGCCTTG | |
| | | ATGAGAAG | |
| CKAP1 | NM_001281 | TCATTGACCACAGTGGCGCCCGCCTT | 1196 |
| | | GGTGAGTATGAGGACGTGTCCCGGGT | |
| | | GGAGAAGTACACGA | |
| CLU | NM_001831 | CCCCAGGATACCTACCACTACCTGCC | 1197 |
| | | CTTCAGCCTGCCCCACCGGAGGCCTC | |
| | | ACTTCTTCTTTCCCAAGTCCCGCA | |
| cMet | NM_000245 | GACATTTCCAGTCCTGCAGTCAATGC | 1198 |
| | | CTCTCTGCCCCACCCTTTGTTCAGTG | |
| | | TGGCTGGTGCCACGACAAATGTGTGC | |
| | | GATCGGAG | |
| cMYC | NM_002467 | TCCCTCCACTCGGAAGGACTATCCTG | 1199 |
| | | CTGCCAAGAGGGTCAAGTTGGACAGT | |
| | | GTCAGAGTCCTGAGACAGATCAGCAA | |
| | | CAACCG | |
| CNN | NM_001299 | TCCACCCTCCTGGCTTTGGCCAGCAT | 1200 |
| | | GGCGAAGACGAAAGGAAACAAGGTGA | |
| | | ACGTGGGAGTGA | |
| COL1A1 | NM_000088 | GTGGCCATCCAGCTGACCTTCCTGCG | 1201 |
| | | CCTGATGTCCACCGAGGCCTCCCAGA | |
| | | ACATCACCTACCACTG | |
| COL1A2 | NM_000089 | CAGCCAAGAACTGGTATAGGAGCTCC | 1202 |
| | | AAGGACAAGAAACACGTCTGGCTAGG | |
| | | AGAAACTATCAATGCTGGCAGCCAGT | |
| | | TT | |
| COL6A3 | NM_004369 | GAGAGCAAGCGAGACATTCTGTTCCT | 1203 |
| | | CTTTGACGGCTCAGCCAATCTTGTGG | |
| | | GCCAGTTCCCTGTT | |
| Contig 51037 | NM_198477 | CGACAGTTGCGATGAAAGTTCTAATC | 1204 |
| | | TCTTCCCTCCTCCTGTTGCTGCCACT | |
| | | AATGCTGATGTCCATGGTCTCTAGCA | |
| | | GCC | |
| COX2 | NM_000963 | TCTGCAGAGTTGGAAGCACTCTATGG | 1205 |
| | | TGACATCGATGCTGTGGAGCTGTATC | |
| | | CTGCCCTTCTGGTAGAAAAGCCTCGG | |
| | | C | |
| COX7C | NM_001867 | ACCTCTGTGGTCCGTAGGAGCCACTA | 1206 |
| | | TGAGGAGGGCCCTGGGAAGAATTTGC | |
| | | CATTTTCAGTGGAAAACAAGTGGTCG | |
| CRABP1 | NM_004378 | AACTTCAAGGTCGGAGAAGGCTTTGA | 1207 |
| | | GGAGGAGACCGTGGACGGACGCAAGT | |
| | | GCAGGAGTTTAGCCA | |
| CRIP2 | NM_001312 | GTGCTACGCCACCCTGTTCGGACCCA | 1208 |
| | | AAGGCGTGAACATCGGGGGCGCGGGC | |
| | | TCCTACATCTACGAGAAGCCCCTG | |
| CRYAB | NM_001885 | GATGTGATTGAGGTGCATGGAAAACA | 1209 |
| | | TGAAGAGCGCCAGGATGAACATGGTT | |
| | | TCATCTCCAGGGAGTTC | |
| CSF1 | NM_000757 | TGCAGCGGCTGATTGACAGTCAGATG | 1210 |
| | | GAGACCTCGTGCCAAATTACATTTGA | |
| | | GTTTGTAGACCAGGAACAGTTG | |
| CSNK1D | NM_001893 | AGCTTTTCCGGAATCTGTTCCATCGC | 1211 |
| | | CAGGGCTTCTCCTATGACTACGTGTT | |
| | | CGACTGGAACATGCTCAAAT | |
| CST7 | NM_003650 | TGGCAGAACTACCTGCAAGAAAAACC | 1212 |
| | | AGCACCTGCGTCTGGATGACTGTGAC | |
| | | TTCCAAACCAACCACACCTTGAAGCA | |
| CTSD | NM_001909 | GTACATGATCCCCTGTGAGAAGGTGT | 1213 |
| | | CCACCCTGCCCGCGATCACACTGAAG | |
| | | CTGGGAGGCAAAGGCTACAAGCTGTC | |
| | | CC | |
| CTSL | NM_001912 | GGGAGGCTTATCTCACTGAGTGAGCA | 1214 |
| | | GAATCTGGTAGACTGCTCTGGGCCTC | |
| | | AAGGCAATGAAGGCTGCAATGG | |
| CTSL2 | NM_001333 | TGTCTCACTGAGCGAGCAGAATCTGG | 1215 |
| | | TGGACTGTTCGCGTCCTCAAGGCAAT | |
| | | CAGGGCTGCAATGGT | |
| CXCR4 | NM_003467 | TGACCGCTTCTACCCCAATGACTTGT | 1216 |
| | | GGGTGGTTGTGTTCCAGTTTCAGCAC | |
| | | ATCATGGTTGGCCTTATCCT | |
| CYBA | NM_000101 | GGTGCCTACTCCATTGTGGCGGGCGT | 1217 |
| | | GTTTGTGTGCCTGCTGGAGTACCCCC | |
| | | GGGGGAAGAGGAAGAAGGGCTCCAC | |
| CYP1B1 | NM_000104 | CCAGCTTTGTGCCTGTCACTATTCCT | 1218 |
| | | CATGCCACCACTGCCAACACCTCTGT | |
| | | CTTGGGCTACCACATTCCC | |
| CYP2C8 | NM_000770 | CCGTGTTCAAGAGGAAGCTCACTGCC | 1219 |
| | | TTGTGGAGGAGTTGAGAAAAACCAAG | |
| | | GCTTCACCCTGTGATCCCACT | |
| CYP3A4 | NM_017460 | AGAACAAGGACAACATAGATCCTTAC | 1220 |
| | | ATATACACACCCTTTGGAAGTGGACC | |
| | | CAGAAACTGCATTGGCATGAGGTTTG | |
| | | C | |
| DDR1 | NM_001954 | CCGTGTGGCTCGCTTTCTGCAGTGCC | 1221 |
| | | GCTTCCTCTTTGCGGGGCCCTGGTTA | |
| | | CTCTTCAGCGAAATCTCC | |
| DIABLO | NM_019887 | CACAATGGCGGCTCTGAAGAGTTGGC | 1222 |
| | | TGTCGCGCAGCGTAACTTCATTCTTC | |
| | | AGGTACAGACAGTGTTTGTGT | |
| DIAPH1 | NM_005219 | CAAGCAGTCAAGGAGAACCAGAAGCG | 1223 |
| | | GCGGGAGACAGAAGAAAAGATGAGGC | |
| | | GAGCAAAACT | |
| DICER1 | NM_177438 | TCCAATTCCAGCATCACTGTGGAGAA | 1224 |
| | | AAGCTGTTTGTCTCCCCAGCATACTT | |
| | | TATCGCCTTCACTGCC | |
| DKFZp564D0462; | NM_198569 | CAGTGCTTCCATGGACAAGTCCTTGT | 1225 |
| | | CAAAACTGGCCCATGCTGATGGAGAT | |
| | | CAAACATCAATCATCCCTGTCCA | |
| DR4 | NM_003844 | TGCACAGAGGGTGTGGGTTACACCAA | 1226 |
| | | TGCTTCCAACAATTTGTTTGCTTGCC | |
| | | TCCCATGTACAGCTTGTAAATCAGAT | |
| | | GAAGA | |
| DR5 | NM_003842 | CTCTGAGACAGTGCTTCGATGACTTT | 1227 |
| | | GCAGACTTGGTGCCCTTTGACTCCTG | |
| | | GGAGCCGCTCATGAGGAAGTTGGGCC | |
| | | TCATGG | |
| DUSP1 | NM_004417 | AGACATCAGCTCCTGGTTCAACGAGG | 1228 |
| | | CCATTGACTTCATAGACTCCATCAAG | |
| | | AATGCTGGAGGAAGGGTGTTTGTC | |
| EEF1D | NM_001960 | CAGAGGATGACGAGGATGATGACATT | 1229 |
| | | GACCTGTTTGGCAGTGACAATGAGGA | |
| | | GGAGGACAAGGAGGCGGCACAG | |
| EGFR | NM_005228 | TGTCGATGGACTTCCAGAACCACCTG | 1230 |
| | | GGCAGCTGCCAAAAGTGTGATCCAAG | |
| | | CTGTCCCAAT | |
| EIF4E | NM_001968 | GATCTAAGATGGCGACTGTCGAACCG | 1231 |
| | | GAAACCACCCCTACTCCTAATCCCCC | |
| | | GACTACAGAAGAGGAGAAAACGGAAT | |
| | | CTAA | |
| EIF4EL3 | NM_004846 | AAGCCGCGGTTGAATGTGCCATGACC | 1232 |
| | | CTCTCCCTCTCTGGATGGCACCATCA | |
| | | TTGAAGCTGGCGTCA | |
| ELP3 | NM_018091 | CTCGGATCCTAGCCCTCGTGCCTCCA | 1233 |
| | | TGGACTCGAGTGTACCGAGTACAGAG | |
| | | GGATATTCCAATGCC | |
| ER2 | NM_001437 | TGGTCCATCGCCAGTTATCACATCTG | 1234 |
| | | TATGCGGAACCTCAAAAGAGTCCCTG | |
| | | GTGTGAAGCAAGATCGCTAGAACA | |
| ErbB3 | NM_001982 | CGGTTATGTCATGCCAGATACACACC | 1235 |
| | | TCAAAGGTACTCCCTCCTCCCGGGAA | |
| | | GGCACCCTTTCTTCAGTGGGTCTCAG | |
| | | TTC | |
| ERBB4 | NM_005235 | TGGCTCTTAATCAGTTTCGTTACCTG | 1236 |
| | | CCTCTGGAGAATTTACGCATTATTCG | |
| | | TGGGACAAAACTTTATGAGGATCGAT | |
| | | ATGCCTTG | |
| ERCC1 | NM_001983 | GTCCAGGTGGATGTGAAAGATCCCCA | 1237 |
| | | GCAGGCCCTCAAGGAGCTGGCTAAGA | |
| | | TGTGTATCCTGGCCG | |
| ERK1 | NM_002746 | ACGGATCACAGTGGAGGAAGCGCTGG | 1238 |
| | | CTCACCCCTACCTGGAGCAGTACTAT | |
| | | GACCCGACGGATGAG | |
| ESPL1 | NM_012291 | ACCCCCAGACCGGATCAGGCAAGCTG | 1239 |
| | | GCCCTCATGTCCCCTTCACGGTGTTT | |
| | | GAGGAAGTCTGCCCTACA | |
| EstR1 | NM_000125 | CGTGGTGCCCCTCTATGACCTGCTGC | 1240 |
| | | TGGAGATGCTGGACGCCCACCGCCTA | |
| | | CATGCGCCCACTAGCC | |
| fas | NM_000043 | GGATTGCTCAACAACCATGCTGGGCA | 1241 |
| | | TCTGGACCCTCCTACCTCTGGTTCTT | |
| | | ACGTCTGTTGCTAGATTATCGTCCAA | |
| | | AAGTGTTAATGCC | |
| fasI | NM_000639 | GCACTTTGGGATTCTTTCCATTATGA | 1242 |
| | | TTCTTTGTTACAGGCACCGAGAATGT | |
| | | TGTATTCAGTGAGGGTCTTCTTACAT | |
| | | GC | |
| FASN | NM_004104 | GCCTCTTCCTGTTCGACGGCTCGCCC | 1243 |
| | | ACCTACGTACTGGCCTACACCCAGAG | |
| | | CTACCGGGCAAAGC | |
| FBXO5 | NM_012177 | GGCTATTCCTCATTTTCTCTACAAAG | 1244 |
| | | TGGCCTCAGTGAACATGAAGAAGGTA | |
| | | GCCTCCTGGAGGAGAATTTCGGTGAC | |
| | | AGTCTACAATCC | |
| FDFT1 | NM_004462 | AAGGAAAGGGTGCCTCATCCCAGCAA | 1245 |
| | | CCTGTCCTTGTGGGTGATGATCACTG | |
| | | TGCTGCTTGTGGCTC | |
| FGFR1 | NM_023109 | CACGGGACATTCACCACATCGACTAC | 1246 |
| | | TATAAAAAGACAACCAACGGCCGACT | |
| | | GCCTGTGAAGTGGATGGCACCC | |
| FHIT | NM_002012 | CCAGTGGAGCGCTTCCATGACCTGCG | 1247 |
| | | TCCTGATGAAGTGGCCGATTTGTTTC | |
| | | AGACGACCCAGAGAG | |
| FIGF | NM_004469 | GGTTCCAGCTTTCTGTAGCTGTAAGC | 1248 |
| | | ATTGGTGGCCACACCACCTCCTTACA | |
| | | AAGCAACTAGAACCTGCGGC | |
| FLJ20354 | NM_017779 | GCGTATGATTTCCCGAATGAGTCAAA | 1249 |
| (DEPDC1 | | ATGTTGATATGCCCAAACTTCATGAT | |
| official) | | GCAATGGGTACGAGGTCACTG | |
| FOS | NM_005252 | CGAGCCCTTTGATGACTTCCTGTTCC | 1250 |
| | | CAGCATCATCCAGGCCCAGTGGCTCT | |
| | | GAGACAGCCCGCTCC | |
| FOXM1 | NM_021953 | CCACCCCGAGCAAATCTGTCCTCCCC | 1251 |
| | | AGAACCCCTGAATCCTGGAGGCTCAC | |
| | | GCCCCCAGCCAAAGTAGGGGGACTGG | |
| | | ATTT | |
| FUS | NM_004960 | GGATAATTCAGACAACAACACCATCT | 1252 |
| | | TTGTGCAAGGCCTGGGTGAGAATGTT | |
| | | ACAATTGAGTCTGTGGCTGATTACTT | |
| | | CA | |
| FYN | NM_002037 | GAAGCGCAGATCATGAAGAAGCTGAA | 1253 |
| | | GCACGACAAGCTGGTCCAGCTCTATG | |
| | | CAGTGGTGTCTGAGGAG | |
| G1P3 | NM_002038 | CCTCCAACTCCTAGCCTCAAGTGATC | 1254 |
| | | CTCCTGTCTCAACCTCCCAAGTAGGA | |
| | | TTACAAGCATGCGCC | |
| GADD45 | NM_001924 | GTGCTGGTGACGAATCCACATTCATC | 1255 |
| | | TCAATGGAAGGATCCTGCCTTAAGTC | |
| | | AACTTATTTGTTTTTGCCGGG | |
| GADD45B | NM_015675 | ACCCTCGACAAGACCACACTTTGGGA | 1256 |
| | | CTTGGGAGCTGGGGCTGAAGTTGCTC | |
| | | TGTACCCATGAACTCCCA | |
| GAGE1 | NM_001468 | AAGGGCAATCACAGTGTTAAAAGAAG | 1257 |
| | | ACATGCTGAAATGTTGCAGGCTGCTC | |
| | | CTATGTTGGAAAATTCTTCATTGAAG | |
| | | TTCTCC | |
| GAPDH | NM_002046 | ATTCCACCCATGGCAAATTCCATGGC | 1258 |
| | | ACCGTCAAGGCTGAGAACGGGAAGCT | |
| | | TGTCATCAATGGAAATCCCATC | |
| GATA3 | NM_002051 | CAAAGGAGCTCACTGTGGTGTCTGTG | 1259 |
| | | TTCCAACCACTGAATCTGGACCCCAT | |
| | | CTGTGAATAAGCCATTCTGACTC | |
| GBP1 | NM_002053 | TTGGGAAATATTTGGGCATTGGTCTG | 1260 |
| | | GCCAAGTCTACAATGTCCCAATATCA | |
| | | AGGACAACCACCCTAGCTTCT | |
| GBP2 | NM_004120 | GCATGGGAACCATCAACCAGCAGGCC | 1261 |
| | | ATGGACCAACTTCACTATGTGACAGA | |
| | | GCTGACAGATCGAATCAAGGCAAACT | |
| | | CCTCA | |
| GCLC | NM_001498 | CTGTTGCAGGAAGGCATTGATCATCT | 1262 |
| | | CCTGGCCCAGCATGTTGCTCATCTCT | |
| | | TTATTAGAGACCCACTGAC | |
| GDF15 | NM_004864 | CGCTCCAGACCTATGATGACTTGTTA | 1263 |
| | | GCCAAAGACTGCCACTGCATATGAGC | |
| | | AGTCCTGGTCCTTCCACTGT | |
| GGPS1 | NM_004837 | CTCCGACGTGGCTTTCCAGTGGCCCA | 1264 |
| | | CAGCATCTATGGAATCCCATCTGTCA | |
| | | TCAATTCTGCCAATTACG | |
| GLRX | NM_002064 | GGAGCTCTGCAGTAACCACAGAACAG | 1265 |
| | | GCCCCATGCTGACGTCCCTCCTCAAG | |
| | | AGCTGGATGGCATTG | |
| GNS | NM_002076 | GGTGAAGGTTGTCTCTTCCGAGGGCC | 1266 |
| | | TTCTGAAGACAGGGCTCTTGAACAGA | |
| | | CAAGTGGAAGGGCTG | |
| GPR56 | NM_005682 | TACCCTTCCATGTGCTGGATCCGGGA | 1267 |
| | | CTCCCTGGTCAGCTACATCACCAACC | |
| | | TGGGCCTCTTCAGC | |
| GPX1 | NM_000581 | GCTTATGACCGACCCCAAGCTCATCA | 1268 |
| | | CCTGGTCTCCGGTGTGTCGCAACGAT | |
| | | GTTGCCTGGAACTTT | |
| GRB7 | NM_005310 | CCATCTGCATCCATCTTGTTTGGGCT | 1269 |
| | | CCCCACCCTTGAGAAGTGCCTCAGAT | |
| | | AATACCCTGGTGGCC | |
| GSK3B | NM_002093 | GACAAGGACGGCAGCAAGGTGACAAC | 1270 |
| | | AGTGGTGGCAACTCCTGGGCAGGGTC | |
| | | CAGACAGGCCACAA | |
| GSR | NM_000637 | GTGATCCCAAGCCCACAATAGAGGTC | 1271 |
| | | AGTGGGAAAAAGTACACCGCCCCACA | |
| | | CATCCTGATCGCCACA | |
| GSTM1 | NM_000561 | AAGCTATGAGGAAAAGAAGTACACGA | 1272 |
| | | TGGGGGACGCTCCTGATTATGACAGA | |
| | | AGCCAGTGGCTGAATGAAAAATTCAA | |
| | | GCTGGGCC | |
| GSTp | NM_000852 | GAGACCCTGCTGTCCCAGAACCAGGG | 1273 |
| | | AGGCAAGACCTTCATTGTGGGAGACC | |
| | | AGATCTCCTTCGCTGACTACAACC | |
| GUS | NM_000181 | CCCACTCAGTAGCCAAGTCACAATGT | 1274 |
| | | TTGGAAAACAGCCCGTTTACTTGAGC | |
| | | AAGACTGATACCACCTGCGTG | |
| HDAC6 | NM_006044 | TCCTGTGCTCTGGAAGCCCTTGAGCC | 1275 |
| | | CTTCTGGGAGGTTCTTGTGAGATCAA | |
| | | CTGAGACCGTGGAG | |
| HER2 | NM_004448 | CGGTGTGAGAAGTGCAGCAAGCCCTG | 1276 |
| | | TGCCCGAGTGTGCTATGGTCTGGGCA | |
| | | TGGAGCACTTGCGAGAGG | |
| HIF1A | NM_001530 | TGAACATAAAGTCTGCAACATGGAAG | 1277 |
| | | GTATTGCACTGCACAGGCCACATTCA | |
| | | CGTATATGATACCAACAGTAACCAAC | |
| | | CTCA | |
| HNF3A | NM_004496 | TCCAGGATGTTAGGAACTGTGAAGAT | 1278 |
| | | GGAAGGGCATGAAACCAGCGACTGGA | |
| | | ACAGCTACTACGCAGACACGC | |
| HRAS | NM_005343 | GGACGAATACGACCCCACTATAGAGG | 1279 |
| | | ATTCCTACCGGAAGCAGGTGGTCATT | |
| | | GATGGGGAGACGTGC | |
| HSPA1A | NM_005345 | CTGCTGCGACAGTCCACTACCTTTTT | 1280 |
| | | CGAGAGTGACTCCCGTTGTCCCAAGG | |
| | | CTTCCCAGAGCGAACCTG | |
| HSPA1B | NM_005346 | GGTCCGCTTCGTCTTTCGAGAGTGAC | 1281 |
| | | TCCCGCGGTCCCAAGGCTTTCCAGAG | |
| | | CGAACCTGTGC | |
| HSPA1L | NM_005527 | GCAGGTGTGATTGCTGGACTTAATGT | 1282 |
| | | GCTAAGAATCATCAATGAGCCCACGG | |
| | | CTGCTGCCATTGCCTATGGT | |
| HSPA5 | NM_005347 | GGCTAGTAGAACTGGATCCCAACACC | 1283 |
| | | AAACTCTTAATTAGACCTAGGCCTCA | |
| | | GCTGCACTGCCCGAAAAGCATTTGGG | |
| | | CAGACC | |
| HSPA9B | NM_004134 | GGCCACTAAAGATGCTGGCCAGATAT | 1284 |
| | | CTGGACTGAATGTGCTTCGGGTGATT | |
| | | AATGAGCCCACAGCTGCT | |
| HSPB1 | NM_001540 | CCGACTGGAGGAGCATAAAAGCGCAG | 1285 |
| | | CCGAGCCCAGCGCCCCGCACTTTTCT | |
| | | GAGCAGACGTCCAGAGCAGAGTCAGC | |
| | | CAGCAT | |
| HSPCA | NM_005348 | CAAAAGGCAGAGGCTGATAAGAACGA | 1286 |
| | | CAAGTCTGTGAAGGATCTGGTCATCT | |
| | | TGCTTTATGAAACTGCGCT | |
| ID1 | NM_002165 | AGAACCGCAAGGTGAGCAAGGTGGAG | 1287 |
| | | ATTCTCCAGCACGTCATCGACTACAT | |
| | | CAGGGACCTTCAGTTGGA | |
| IFITM1 | NM_003641 | CACGCAGAAAACCACACTTCTCAAAC | 1288 |
| | | CTTCACTCAACACTTCCTTCCCCAAA | |
| | | GCCAGAAGATGCACAAGGAGGAACAT | |
| | | G | |
| IGF1R | NM_000875 | GCATGGTAGCCGAAGATTTCACAGTC | 1289 |
| | | AAAATCGGAGATTTTGGTATGACGCG | |
| | | AGATATCTATGAGACAGACTATTACC | |
| | | GGAAA | |
| IGFBP2 | NM_000597 | GTGGACAGCACCATGAACATGTTGGG | 1290 |
| | | CGGGGGAGGCAGTGCTGGCCGGAAGC | |
| | | CCCTCAAGTCGGGTATGAAGG | |
| IGFBP3 | NM_000598 | ACGCACCGGGTGTCTGATCCCAAGTT | 1291 |
| | | CCACCCCCTCCATTCAAAGATAATCA | |
| | | TCATCAAGAAAGGGCA | |
| IGFBP5 | NM_000599 | TGGACAAGTACGGGATGAAGCTGCCA | 1292 |
| | | GGCATGGAGTACGTTGACGGGGACTT | |
| | | TCAGTGCCACACCTTCG | |
| IL2RA | NM_000417 | TCTGCGTGGTTCCTTTCTCAGCCGCT | 1293 |
| | | TCTGACTGCTGATTCTCCCGTTCACG | |
| | | TTGCCTAATAAACATCCTTCAA | |
| IL6 | NM_000600 | CCTGAACCTTCCAAAGATGGCTGAAA | 1294 |
| | | AAGATGGATGCTTCCAATCTGGATTC | |
| | | AATGAGGAGACTTGCCTGGT | |
| IL-7 | NM_000880 | GCGGTGATTCGGAAATTCGCGAATTC | 1295 |
| | | CTCTGGTCCTCATCCAGGTGCGCGGG | |
| | | AAGCAGGTGCCCAGGAGAG | |
| IL-8 | NM_000584 | AAGGAACCATCTCACTGTGTGTAAAC | 1296 |
| | | ATGACTTCCAAGCTGGCCGTGGCTCT | |
| | | CTTGGCAGCCTTCCTGAT | |
| IL8RB | NM_001557 | CCGCTCCGTCACTGATGTCTACCTGC | 1297 |
| | | TGAACCTAGCCTTGGCCGACCTACTC | |
| | | TTTGCCCTGACCTTGC | |
| ILK | NM_001014794 | CTCAGGATTTTCTCGCATCCAAATGT | 1298 |
| | | GCTCCCAGTGCTAGGTGCCTGCCAGT | |
| | | CTCCACCTGCTCCT | |
| ILT-2 | NM_006669 | AGCCATCACTCTCAGTGCAGCCAGGT | 1299 |
| | | CCTATCGTGGCCCCTGAGGAGACCCT | |
| | | GACTCTGCAGT | |
| INCENP | NM_020238 | GCCAGGATACTGGAGTCCATCACAGT | 1300 |
| | | GAGCTCCCTGATGGCTACACCCCAGG | |
| | | ACCCCAAGGGTCAAG | |
| IRAK2 | NM_001570 | GGATGGAGTTCGCCTCCTACGTGATC | 1301 |
| | | ACAGACCTGACCCAGCTGCGGAAGAT | |
| | | CAAGTCCATGGAGCG | |
| IRS1 | NM_005544 | CCACAGCTCACCTTCTGTCAGGTGTC | 1302 |
| | | CATCCCAGCTCCAGCCAGCTCCCAGA | |
| | | GAGGAAGAGACTGGCACTGAGG | |
| ITGB1 | NM_002211 | TCAGAATTGGATTTGGCTCATTTGTG | 1303 |
| | | GAAAAGACTGTGATGCCTTACATTAG | |
| | | CACAACACCAGCTAAGCTCAGG | |
| K-Alpha-1 | NM_006082 | TGAGGAAGAAGGAGAGGAATACTAAT | 1304 |
| | | TATCCATTCCTTTTGGCCCTGCAGCA | |
| | | TGTCATGCTCCCAGAATTTCAG | |
| KDR | NM_002253 | GAGGACGAAGGCCTCTACACCTGCCA | 1305 |
| | | GGCATGCAGTGTTCTTGGCTGTGCAA | |
| | | AAGTGGAGGCATTTTT | |
| Ki-67 | NM_002417 | CGGACTTTGGGTGCGACTTGACGAGC | 1306 |
| | | GGTGGTTCGACAAGTGGCCTTGCGGG | |
| | | CCGGATCGTCCCAGTGGAAGAGTTGT | |
| | | AA | |
| KIF11 | NM_004523 | TGGAGGTTGTAAGCCAATGTTGTGAG | 1307 |
| | | GCTTCAAGTTCAGACATCACTGAGAA | |
| | | ATCAGATGGACGTAAGGCA | |
| KIF22 | NM_007317 | CTAAGGCACTTGCTGGAAGGGCAGAA | 1308 |
| | | TGCCAGTGTGCTTGCCTATGGACCCA | |
| | | CAGGAGCTGGGAAGA | |
| KIF2C | NM_006845 | AATTCCTGCTCCAAAAGAAAGTCTTC | 1309 |
| | | GAAGCCGCTCCACTCGCATGTCCACT | |
| | | GTCTCAGAGCTTCGCATCACG | |
| KIFC1 | NM_002263 | CCACAGGGTTGAAGAACCAGAAGCCA | 1310 |
| | | GTTCCTGCTGTTCCTGTCCAGAAGTC | |
| | | TGGCACATCAGGTG | |
| KLK10 | NM_002776 | GCCCAGAGGCTCCATCGTCCATCCTC | 1311 |
| | | TTCCTCCCCAGTCGGCTGAACTCTCC | |
| | | CCTTGTCTGCACTGTTCAAACCTCTG | |
| KNS2 | NM_005552 | CAAACAGAGGGTGGCAGAAGTGCTCA | 1312 |
| | | ATGACCCTGAGAACATGGAGAAGCGC | |
| | | AGGAGCCGTGAGAGCCTC | |
| KNTC1 | NM_014708 | AGCCGAGGCTTTGTTGAAGAAGCTTC | 1313 |
| | | ATATCCAGTACCGGCGATCGGGCACA | |
| | | GAAGCTGTGCTCATAGCCCA | |
| KNTC2 | NM_006101 | ATGTGCCAGTGAGCTTGAGTCCTTGG | 1314 |
| | | AGAAACACAAGCACCTGCTAGAAAGT | |
| | | ACTGTTAACCAGGGGCTCA | |
| KRT14 | NM_000526 | GGCCTGCTGAGATCAAAGACTACAGT | 1315 |
| | | CCCTACTTCAAGACCATTGAGGACCT | |
| | | GAGGAACAAGATTCTCACAGCCACAG | |
| | | TGGAC | |
| KRT17 | NM_000422 | CGAGGATTGGTTCTTCAGCAAGACAG | 1316 |
| | | AGGAACTGAACCGCGAGGTGGCCACC | |
| | | AACAGTGAGCTGGTGCAGAGT | |
| KRT19 | NM_002276 | TGAGCGGCAGAATCAGGAGTACCAGC | 1317 |
| | | GGCTCATGGACATCAAGTCGCGGCTG | |
| | | GAGCAGGAGATTGCCACCTACCGCA | |
| KRT5 | NM_000424 | TCAGTGGAGAAGGAGTTGGACCAGTC | 1318 |
| | | AACATCTCTGTTGTCACAAGCAGTGT | |
| | | TTCCTCTGGATATGGCA | |
| L1CAM | NM_000425 | CTTGCTGGCCAATGCCTACATCTACG | 1319 |
| | | TTGTCCAGCTGCCAGCCAAGATCCTG | |
| | | ACTGCGGACAATCA | |
| LAMC2 | NM_005562 | ACTCAAGCGGAAATTGAAGCAGATAG | 1320 |
| | | GTCTTATCAGCACAGTCTCCGCCTCC | |
| | | TGGATTCAGTGTCTCGGCTTCAGGGA | |
| | | GT | |
| LAPTM4B | NM_018407 | AGCGATGAAGATGGTCGCGCCCTGGA | 1321 |
| | | CGCGGTTCTACTCCAACAGCTGCTGC | |
| | | TTGTGCTGCCATGTC | |
| LIMK1 | NM_016735 | GCTTCAGGTGTTGTGACTGCAGTGCC | 1322 |
| | | TCCCTGTCGCACCAGTACTATGAGAA | |
| | | GGATGGGCAGCTCTT | |
| LIMK2 | NM_005569 | CTTTGGGCCAGGAGGAATCTGTTACT | 1323 |
| | | CGAATCCACCCAGGAACTCCCTGGCA | |
| | | GTGGATTGTGGGAG | |
| MAD1L1 | NM_003550 | AGAAGCTGTCCCTGCAAGAGCAGGAT | 1324 |
| | | GCAGCGATTGTGAAGAACATGAAGTC | |
| | | TGAGCTGGTACGGCT | |
| MAD2L1 | NM_002358 | CCGGGAGCAGGGAATCACCCTGCGCG | 1325 |
| | | GGAGCGCCGAAATCGTGGCCGAGTTC | |
| | | TTCTCATTCGGCATCAACAGCAT | |
| MAD2L1BP | NM_014628 | CTGTCATGTGGCAGACCTTCCATCCG | 1326 |
| | | AACCACGGCTTGGGAAGACTACATTT | |
| | | GGTTCCAGGCACCAGTGACATTTA | |
| MAD2L2 | NM_006341 | CCTCAGAAATTGCCAGGACTTCTTTC | 1327 |
| | | CCGTGATCTTCAGCAAAGCCTCCGAG | |
| | | TACTTGCAGCTGGTCTTTGG | |
| MAGE2 | NM_005361 | CCTCAGAAATTGCCAGGACTTCTTTC | 1328 |
| | | CCGTGATCTTCAGCAAAGCCTCCGAG | |
| | | TACTTGCAGCTGGTCTTTGG | |
| MAGE6 | NM_005363 | AGGACTCCAGCAACCAAGAAGAGGAG | 1329 |
| | | GGGCCAAGCACCTTCCCTGACCTGGA | |
| | | GTCTGAGTTCCAAGCAGCACTC | |
| MAP2 | NM_002374 | CGGACCACCAGGTCAGAGCCAATTCG | 1330 |
| | | CAGAGCAGGGAAGAGTGGTACCTCAA | |
| | | CACCCACTACCCCTG | |
| MAP2K3 | NM_002756 | GCCCTCCAATGTCCTTATCAACAAGG | 1331 |
| | | AGGGCCATGTGAAGATGTGTGACTTT | |
| | | GGCATCAGTGGCTAC | |
| MAP4 | NM_002375 | GCCGGTCAGGCACACAAGGGGCCCTT | 1332 |
| | | GGAGCGTGGACTGGTTGGTTTTGCCA | |
| | | TTTTGTTGTGTGTATGCTGC | |
| MAP6 | NM_033063 | CCCTCAACCGGCAAATCCGCGAGGAG | 1333 |
| | | GTGGCGAGTGCAGTGAGCAGCTCCTA | |
| | | CAGGAATGAATTCAGGGCATGGACG | |
| MAPK14 | NM_139012 | TGAGTGGAAAAGCCTGACCTATGATG | 1334 |
| | | AAGTCATCAGCTTTGTGCCACCACCC | |
| | | CTTGACCAAGAAGAGATGGAGTCC | |
| MAPK8 | NM_002750 | CAACACCCGTACATCAATGTCTGGTA | 1335 |
| | | TGATCCTTCTGAAGCAGAAGCTCCAC | |
| | | CACCAAAGATCCCTGACAAGCAGTTA | |
| | | GATGA | |
| MAPRE1 | NM_012325 | GACCTTGGAACCTTTGGAACCTGCTG | 1336 |
| | | TCAACAGGTCTTACAGGGCTGCTTGA | |
| | | ACCCTCATAGGCCTAGG | |
| MAPT | NM_016835 | CACAAGCTGACCTTCCGCGAGAACGC | 1337 |
| | | CAAAGCCAAGACAGACCACGGGGCGG | |
| | | AGATCGTGTACAAGT | |
| Maspin | NM_002639 | CAGATGGCCACTTTGAGAACATTTTA | 1338 |
| | | GCTGACAACAGTGTGAACGACCAGAC | |
| | | CAAAATCCTTGTGGTTAATGCTGCC | |
| MCL1 | NM_021960 | CTTCGGAAACTGGACATCAAAAACGA | 1339 |
| | | AGACGATGTGAAATCGTTGTCTCGAG | |
| | | TGATGATCCATGTTTTCAGCGAC | |
| MCM2 | NM_004526 | GACTTTTGCCCGCTACCTTTCATTCC | 1340 |
| | | GGCGTGACAACAATGAGCTGTTGCTC | |
| | | TTCATACTGAAGCAGTTAGTGGC | |
| MCM6 | NM_005915 | TGATGGTCCTATGTGTCACATTCATC | 1341 |
| | | ACAGGTTTCATACCAACACAGGCTTC | |
| | | AGCACTTCCTTTGGTGTGTTTCCTGT | |
| | | CCCA | |
| MCP1 | NM_002982 | CGCTCAGCCAGATGCAATCAATGCCC | 1342 |
| | | CAGTCACCTGCTGTTATAACTTCACC | |
| | | AATAGGAAGATCTCAGTGC | |
| MGMT | NM_002412 | GTGAAATGAAACGCACCACACTGGAC | 1343 |
| | | AGCCCTTTGGGGAAGCTGGAGCTGTC | |
| | | TGGTTGTGAGCAGGGTC | |
| MMP12 | NM_002426 | CCAACGCTTGCCAAATCCTGACAATT | 1344 |
| | | CAGAACCAGCTCTCTGTGACCCCAAT | |
| | | TTGAGTTTTGATGCTGTCACTACCGT | |
| MMP2 | NM_004530 | CCATGATGGAGAGGCAGACATCATGA | 1345 |
| | | TCAACTTTGGCCGCTGGGAGCATGGC | |
| | | GATGGATACCCCTTTGACGGTAAGGA | |
| | | CGGACTCC | |
| MMP9 | NM_004994 | GAGAACCAATCTCACCGACAGGCAGC | 1346 |
| | | TGGCAGAGGAATACCTGTACCGCTAT | |
| | | GGTTACACTCGGGTG | |
| MRE11A | NM_005590 | GCCATGCTGGCTCAGTCTGAGCTGTG | 1347 |
| | | GGCCACATCAGCTAGTGGCTCTTCTC | |
| | | ATGCATCAGTTAGGTGGGTCTGGGTG | |
| MRP1 | NM_004996 | TCATGGTGCCCGTCAATGCTGTGATG | 1348 |
| | | GCGATGAAGACCAAGACGTATCAGGT | |
| | | GGCCCACATGAAGAGCAAAGACAATC | |
| | | G | |
| MRP2 | NM_000392 | AGGGGATGACTTGGACACATCTGCCA | 1349 |
| | | TTCGACATGACTGCAATTTTGACAAA | |
| | | GCCATGCAGTTTT | |
| MRP3 | NM_003786 | TCATCCTGGCGATCTACTTCCTCTGG | 1350 |
| | | CAGAACCTAGGTCCCTCTGTCCTGGC | |
| | | TGGAGTCGCTTTCATGGTCTTGCTGA | |
| | | TTCCACTCAACGG | |
| MSH3 | NM_002439 | TGATTACCATCATGGCTCAGATTGGC | 1351 |
| | | TCCTATGTTCCTGCAGAAGAAGCGAC | |
| | | AATTGGGATTGTGGATGGCATTTTCA | |
| | | CAAG | |
| MUC1 | NM_002456 | GGCCAGGATCTGTGGTGGTACAATTG | 1352 |
| | | ACTCTGGCCTTCCGAGAAGGTACCAT | |
| | | CAATGTCCACGACGTGGAG | |
| MX1 | NM_002462 | GAAGGAATGGGAATCAGTCATGAGCT | 1353 |
| | | AATCACCCTGGAGATCAGCTCCCGAG | |
| | | ATGTCCCGGATCTGACTCTAATAGAC | |
| MYBL2 | NM_002466 | GCCGAGATCGCCAAGATGTTGCCAGG | 1354 |
| | | GAGGACAGACAATGCTGTGAAGAATC | |
| | | ACTGGAACTCTACCATCAAAAG | |
| MYH11 | NM_002474 | CGGTACTTCTCAGGGCTAATATATAC | 1355 |
| | | GTACTCTGGCCTCTTCTGCGTGGTGG | |
| | | TCAACCCCTATAAACACCTGCCCATC | |
| | | TACTCGG | |
| NEK2 | NM_002497 | GTGAGGCAGCGCGACTCTGGCGACTG | 1356 |
| | | GCCGGCCATGCCTTCCCGGGCTGAGG | |
| | | ACTATGAAGTGTTGTACACCATTGGC | |
| | | A | |
| NFKBp50 | NM_003998 | CAGACCAAGGAGATGGACCTCAGCGT | 1357 |
| | | GGTGCGGCTCATGTTTACAGCTTTTC | |
| | | TTCCGGATAGCACTGGCAGCT | |
| NFKBp65 | NM_021975 | CTGCCGGGATGGCTTCTATGAGGCTG | 1358 |
| | | AGCTCTGCCCGGACCGCTGCATCCAC | |
| | | AGTTTCCAGAACCTGG | |
| NME6 | NM_005793 | CACTGACACCCGCAACACCACCCATG | 1359 |
| | | GTTCGGACTCTGTGGTTTCAGCCAGC | |
| | | AGAGAGATTGCAGCC | |
| NPC2 | NM_006432 | CTGCTTCTTTCCCGAGCTTGGAACTT | 1360 |
| | | CGTTATCCGCGATGCGTTTCCTGGCA | |
| | | GCTACATTCCTGCT | |
| NPD009 | NM_020686 | GGCTGTGGCTGAGGCTGTAGCATCTC | 1361 |
| (ABAT official) | | TGCTGGAGGTGAGACACTCTGGGAAC | |
| | | TGATTTGACCTCGAATGCTCC | |
| NTSR2 | NM_012344 | CGGACCTGAATGTAATGCAAGAATGA | 1362 |
| | | ACAGAACAAGCAAAATGACCAGCTGC | |
| | | TTAGTCACCTGGCAAAG | |
| NUSAP1 | NM_016359 | CAAAGGAAGAGCAACGGAAGAAACGC | 1363 |
| | | GAGCAAGAACGAAAGGAGAAGAAAGC | |
| | | AAAGGTTTTGGGAAT | |
| p21 | NM_000389 | TGGAGACTCTCAGGGTCGAAAACGGC | 1364 |
| | | GGCAGACCAGCATGACAGATTTCTAC | |
| | | CACTCCAAACGCC | |
| p27 | NM_004064 | CGGTGGACCACGAAGAGTTAACCCGG | 1365 |
| | | GACTTGGAGAAGCACTGCAGAGACAT | |
| | | GGAAGAGGCGAGCC | |
| PCTK1 | NM_006201 | TCACTACCAGCTGACATCCGGCTGCC | 1366 |
| | | TGAGGGCTACCTGGAGAAGCTGACCC | |
| | | TCAATAGCCCCATCT | |
| PDGFRb | NM_002609 | CCAGCTCTCCTTCCAGCTACAGATCA | 1367 |
| | | ATGTCCCTGTCCGAGTGCTGGAGCTA | |
| | | AGTGAGAGCCACCC | |
| PFDN5 | NM_145897 | GAGAAGCACGCCATGAAACAGGCCGT | 1368 |
| | | CATGGAAATGATGAGTCAGAAGATTC | |
| | | AGCAGCTCACAGCC | |
| PGK1 | NM_000291 | AGAGCCAGTTGCTGTAGAACTCAAAT | 1369 |
| | | CTCTGCTGGGCAAGGATGTTCTGTTC | |
| | | TTGAAGGACTGTGTAGGCCCAG | |
| PHB | NM_002634 | GACATTGTGGTAGGGGAAGGGACTCA | 1370 |
| | | TTTTCTCATCCCGTGGGTACAGAAAC | |
| | | CAATTATCTTTGACTGCCG | |
| PI3KC2A | NM_002645 | ATACCAATCACCGCACAAACCCAGGC | 1371 |
| | | TATTTGTTAAGTCCAGTCACAGCGCA | |
| | | AAGAAACATATGCGGAGAAAATGCTA | |
| | | GTGTG | |
| PIM1 | NM_002648 | CTGCTCAAGGACACCGTCTACACGGA | 1372 |
| | | CTTCGATGGGACCCGAGTGTATAGCC | |
| | | CTCCAGAGTGGATCC | |
| PIM2 | NM_006875 | TGGGGACATTCCCTTTGAGAGGGACC | 1373 |
| | | AGGAGATTCTGGAAGCTGAGCTCCAC | |
| | | TTCCCAGCCCATGTC | |
| PLAUR | NM_002659 | CCCATGGATGCTCCTCTGAAGAGACT | 1374 |
| | | TTCCTCATTGACTGCCGAGGCCCCAT | |
| | | GAATCAATGTCTGGTAGCCACCGG | |
| PLD3 | NM_012268 | CCAAGTTCTGGGTGGTGGACCAGACC | 1375 |
| | | CACTTCTACCTGGGCAGTGCCAACAT | |
| | | GGACTGGCGTTCAC | |
| PLK | NM_005030 | AATGAATACAGTATTCCCAAGCACAT | 1376 |
| | | CAACCCCGTGGCCGCCTCCCTCATCC | |
| | | AGAAGATGCTTCAGACA | |
| PMS1 | NM_000534 | CTTACGGTTTTCGTGGAGAAGCCTTG | 1377 |
| | | GGGTCAATTTGTTGTATAGCTGAGGT | |
| | | TTTAATTACAACAAGAACGGCTGCT | |
| PMS2 | NM_000535 | GATGTGGACTGCCATTCAAACCAGGA | 1378 |
| | | AGATACCGGATGTAAATTTCGAGTTT | |
| | | TGCCTCAGCCAACTAATCTCGCA | |
| PP591 | NM_025207 | CCACATACCGTCCAGCCTATCTACTG | 1379 |
| | | GAGAACGAAGAAGAGGAGCGGAACTC | |
| | | CCGCACATGACCTC | |
| PPP2CA | NM_002715 | GCAATCATGGAACTTGACGATACTCT | 1380 |
| | | AAAATACTCTTTCTTGCAGTTTGACC | |
| | | CAGCACCTCGTAGAGGCGAGCCACAT | |
| PR | NM_000926 | GCATCAGGCTGTCATTATGGTGTCCT | 1381 |
| | | TACCTGTGGGAGCTGTAAGGTCTTCT | |
| | | TTAAGAGGGCAATGGAAGGGCAGCAC | |
| | | AACTACT | |
| PRDX1 | NM_002574 | AGGACTGGGACCCATGAACATTCCTT | 1382 |
| | | TGGTATCAGACCCGAAGCGCACCATT | |
| | | GCTCAGGATTATGGG | |
| PRDX2 | NM_005809 | GGTGTCCTTCGCCAGATCACTGTTAA | 1383 |
| | | TGATTTGCCTGTGGGACGCTCCGTGG | |
| | | ATGAGGCTCTGCGGCTG | |
| PRKCA | NM_002737 | CAAGCAATGCGTCATCAATGTCCCCA | 1384 |
| | | GCCTCTGCGGAATGGATCACACTGAG | |
| | | AAGAGGGGGCGGATTTAC | |
| PRKCD | NM_006254 | CTGACACTTGCCGCAGAGAATCCCTT | 1385 |
| | | TCTCACCCACCTCATCTGCACCTTCC | |
| | | AGACCAAGGACCACCT | |
| PRKCG | NM_002739 | GGGTTCTAGACGCCCCTCCCAAGCGT | 1386 |
| | | TCCTGGCCTTCTGAACTCCATACAGC | |
| | | CTCTACAGCCGTCC | |
| PRKCH | NM_006255 | CTCCACCTATGAGCGTCTGTCTCTGT | 1387 |
| | | GGGCTTGGGATGTTAACAGGAGCCAA | |
| | | AAGGAGGGAAAGTGTG | |
| pS2 | NM_003225 | GCCCTCCCAGTGTGCAAATAAGGGCT | 1388 |
| | | GCTGTTTCGACGACACCGTTCGTGGG | |
| | | GTCCCCTGGTGCTTCTATCCTAATAC | |
| | | CATCGACG | |
| PTEN | NM_000314 | TGGCTAAGTGAAGATGACAATCATGT | 1389 |
| | | TGCAGCAATTCACTGTAAAGCTGGAA | |
| | | AGGGACGAACTGGTGTAATGATATGT | |
| | | GCA | |
| PTPD1 | NM_007039 | CGCTTGCCTAACTCATACTTTCCCGT | 1390 |
| | | TGACACTTGATCCACGCAGCGTGGCA | |
| | | CTGGGACGTAAGTGGCGCAGTCTGAA | |
| | | TGG | |
| PTTG1 | NM_004219 | GGCTACTCTGATCTATGTTGATAAGG | 1391 |
| | | AAAATGGAGAACCAGGCACCCGTGTG | |
| | | GTTGCTAAGGATGGGCTGAAGC | |
| RAB27B | NM_004163 | GGGACACTGCGGGACAAGAGCGGTTC | 1392 |
| | | CGGAGTCTCACCACTGCATTTTTCAG | |
| | | AGACGCCATGGGC | |
| RAB31 | NM_006868 | CTGAAGGACCCTACGCTCGGTGGCCT | 1393 |
| | | GGCACCTCACTTTGAGAAGAGTGAGC | |
| | | ACACTGGCTTTGCAT | |
| RAB6C | NM_032144 | GCGACAGCTCCTCTAGTTCCACCATG | 1394 |
| | | TCCGCGGGCGGAGACTTCGGGAATCC | |
| | | GCTGAGGAAATTCAAGCTGGTGTTCC | |
| RAD1 | NM_002853 | GAGGAGTGGTGACAGTCTGCAAAATC | 1395 |
| | | AATACACAGGAACCTGAGGAGACCCT | |
| | | GGACTTTGATTTCTGCAGC | |
| RAD54L | NM_003579 | AGCTAGCCTCAGTGACACACATGACA | 1396 |
| | | GGTTGCACTGCCGACGTTGTGTCAAC | |
| | | AGCCGTCAGATCCGG | |
| RAF1 | NM_002880 | CGTCGTATGCGAGAGTCTGTTTCCAG | 1397 |
| | | GATGCCTGTTAGTTCTCAGCACAGAT | |
| | | ATTCTACACCTCACGCCTTCA | |
| RALBP1 | NM_006788 | GGTGTCAGATATAAATGTGCAAATGC | 1398 |
| | | CTTCTTGCTGTCCTGTCGGTCTCAGT | |
| | | ACGTTCACTTTATAGCTGCTGGCAAT | |
| | | ATCGAA | |
| RAP1GDS1 | NM_021159 | TGTGGATGCTGGATTGATTTCACCAC | 1399 |
| | | TGGTGCAGCTGCTAAATAGCAAAGAC | |
| | | CAGGAAGTGCTGCTT | |
| RASSF1 | NM_007182 | AGTGGGAGACACCTGACCTTTCTCAA | 1400 |
| | | GCTGAGATTGAGCAGAAGATCAAGGA | |
| | | GTACAATGCCCAGATCA | |
| RB1 | NM_000321 | CGAAGCCCTTACAAGTTTCCTAGTTC | 1401 |
| | | ACCCTTACGGATTCCTGGAGGGAACA | |
| | | TCTATATTTCACCCCTGAAGAGTCC | |
| RBM17 | NM_032905 | CCCAGTGTACGAGGAACAAGACAGAC | 1402 |
| | | CGAGATCTCCAACCGGACCTAGCAAC | |
| | | TCCTTCCTCGCTAA | |
| RCC1 | NM_001269 | GGGCTGGGTGAGAATGTGATGGAGAG | 1403 |
| | | GAAGAAGCCGGCCCTGGTATCCATTC | |
| | | CGGAGGATGTTGTG | |
| REG1A | NM_002909 | CCTACAAGTCCTGGGGCATTGGAGCC | 1404 |
| | | CCAAGCAGTGTTAATCCTGGCTACTG | |
| | | TGTGAGCCTGACCTCA | |
| RELB | NM_006509 | GCGAGGAGCTCTACTTGCTCTGCGAC | 1405 |
| | | AAGGTGCAGAAAGAGGACATATCAGT | |
| | | GGTGTTCAGCAGGGC | |
| RhoB | NM_004040 | AAGCATGAACAGGACTTGACCATCTT | 1406 |
| | | TCCAACCCCTGGGGAAGACATTTGCA | |
| | | ACTGACTTGGGGAGG | |
| rhoC | NM_175744 | CCCGTTCGGTCTGAGGAAGGCCGGGA | 1407 |
| | | CATGGCGAACCGGATCAGTGCCTTTG | |
| | | GCTACCTTGAGTGCTC | |
| RIZ1 | NM_012231 | CCAGACGAGCGATTAGAAGCGGCAGC | 1408 |
| | | TTGTGAGGTGAATGATTTGGGGGAAG | |
| | | AGGAGGAGGAGGAAGAGGAGGA | |
| ROCK1 | NM_005406 | TGTGCACATAGGAATGAGCTTCAGAT | 1409 |
| | | GCAGTTGGCCAGCAAAGAGAGTGATA | |
| | | TTGAGCAATTGCGTGCTAAAC | |
| RPL37A | NM_000998 | GATCTGGCACTGTGGTTCCTGCATGA | 1410 |
| | | AGACAGTGGCTGGCGGTGCCTGGACG | |
| | | TACAATACCACTTCCGCTGTCA | |
| RPLPO | NM_001002 | CCATTCTATCATCAACGGGTACAAAC | 1411 |
| | | GAGTCCTGGCCTTGTCTGTGGAGACG | |
| | | GATTACACCTTCCCACTTGCTGA | |
| RPN2 | NM_002951 | CTGTCTTCCTGTTGGCCCTGACAATC | 1412 |
| | | ATAGCCAGCACCTGGGCTCTGACGCC | |
| | | CACTCACTACCTCAC | |
| RPS6KB1 | NM_003161 | GCTCATTATGAAAAACATCCCAAACT | 1413 |
| | | TTAAAATGCGAAATTATTGGTTGGTG | |
| | | TGAAGAAAGCCAGACAACTTCTGTTT | |
| | | CTT | |
| RXRA | NM_002957 | GCTCTGTTGTGTCCTGTTGCCGGCTC | 1414 |
| | | TGGCCTTCCTGTGACTGACTGTGAAG | |
| | | TGGCTTCTCCGTAC | |
| RXRB | NM_021976 | CGAGGAGATGCCTGTGGACAGGATCC | 1415 |
| | | TGGAGGCAGAGCTTGCTGTGGAACAG | |
| | | AAGAGTGACCAGGGCGTTG | |
| S100A10 | NM_002966 | ACACCAAAATGCCATCTCAAATGGAA | 1416 |
| | | CACGCCATGGAAACCATGATGTTTAC | |
| | | ATTTCACAAATTCGCTGGGGATAAA | |
| SEC61A | NM_013336 | CTTCTGAGCCCGTCTCCCGGACAGGT | 1417 |
| | | TGAGGAAGCTGCTCCAGAAGCGCCTC | |
| | | GGAAGGGGAGCTCTC | |
| SEMA3F | NM_004186 | CGCGAGCCCCTCATTATACACTGGGC | 1418 |
| | | AGCCTCCCCACAGCGCATCGAGGAAT | |
| | | GCGTGCTCTCAGGCAAGGATGTCAAC | |
| | | GGCGAGTG | |
| SFN | NM_006142 | GAGAGAGCCAGTCTGATCCAGAAGGC | 1419 |
| | | CAAGCTGGCAGAGCAGGCCGAACGCT | |
| | | ATGAGGACATGGCAGCCT | |
| SGCB | NM_000232 | CAGTGGAGACCAGTTGGGTAGTGGTG | 1420 |
| | | ACTGGGTACGCTACAAGCTCTGCATG | |
| | | TGTGCTGATGGGACGCTCTTCAAGG | |
| SGK | NM_005627 | TCCGCAAGACACCTCCTGGAGGGCCT | 1421 |
| | | CCTGCAGAAGGACAGGACAAAGCGGC | |
| | | TCGGGGCCAAGGATGACTTCA | |
| SGKL | NM_170709 | TGCATTCGTTGGTTTCTCTTATGCAC | 1422 |
| | | CTCCTTCAGAAGACTTATTTTTGTGA | |
| | | GCAGTTTGCCATTCAGAAA | |
| SHC1 | NM_003029 | CCAACACCTTCTTGGCTTCTGGGACC | 1423 |
| | | TGTGTTCTTGCTGAGCACCCTCTCCG | |
| | | GTTTGGGTTGGGATAACAG | |
| SIR2 | NM_012238 | AGCTGGGGTGTCTGTTTCATGTGGAA | 1424 |
| | | TACCTGACTTCAGGTCAAGGGATGGT | |
| | | ATTTATGCTCGCCTTGCTGT | |
| SLC1A3 | NM_004172 | GTGGGGAGCCCATCATCTCGCCAAGC | 1425 |
| | | CATCACAGGCTCTGCATACACATGCA | |
| | | CTCAGTGTGGACTGG | |
| SLC25A3 | NM_213611 | TCTGCCAGTGCTGAATTCTTTGCTGA | 1426 |
| | | CATTGCCCTGGCTCCTATGGAAGCTG | |
| | | CTAAGGTTCGAA | |
| SLC35B1 | NM_005827 | CCCAACTCAGGTCCTTGGTAAATCCT | 1427 |
| | | GCAAGCCAATCCCAGTCATGCTCCTT | |
| | | GGGGTGACCCTCTTG | |
| SLC7A11 | NM_014331 | AGATGCATACTTGGAAGCACAGTCAT | 1428 |
| | | ATCACACTGGGAGGCAATGCAATGTG | |
| | | GTTACCTGGTCCTAGGTT | |
| SLC7A5 | NM_003486 | GCGCAGAGGCCAGTTAAAGTAGATCA | 1429 |
| | | CCTCCTCGAACCCACTCCGGTTCCCC | |
| | | GCAACCCACAGCTCAGCT | |
| SNAI2 | NM_003068 | GGCTGGCCAAACATAAGCAGCTGCAC | 1430 |
| | | TGCGATGCCCAGTCTAGAAAATCTTT | |
| | | CAGCTGTAAATACTGTGACAAGGA | |
| SNCA | NM_007308 | AGTGACAAATGTTGGAGGAGCAGTGG | 1431 |
| | | TGACGGGTGTGACAGCAGTAGCCCAG | |
| | | AAGACAGTGGAGGG | |
| SNCG | NM_003087 | ACCCACCATGGATGTCTTCAAGAAGG | 1432 |
| | | GCTTCTCCATCGCCAAGGAGGGCGTG | |
| | | GTGGGTGCGGTGGAAAAGACCAAGCA | |
| | | GG | |
| SOD1 | NM_000454 | TGAAGAGAGGCATGTTGGAGACTTGG | 1433 |
| | | GCAATGTGACTGCTGACAAAGATGGT | |
| | | GTGGCCGATGTGTCTATT | |
| SRC | NM_005417 | TGAGGAGTGGTATTTTGGCAAGATCA | 1434 |
| | | CCAGACGGGAGTCAGAGCGGTTACTG | |
| | | CTCAATGCAGAGAACCCGAGAG | |
| SRI | NM_003130 | ATACAGCACCAATGGAAAGATCACCT | 1435 |
| | | TCGACGACTACATCGCCTGCTGCGTC | |
| | | AAACTGAGGGCTCTTACAGACA | |
| STAT1 | NM_007315 | GGGCTCAGCTTTCAGAAGTGCTGAGT | 1436 |
| | | TGGCAGTTTTCTTCTGTCACCAAAAG | |
| | | AGGTCTCAATGTGGACCAGCTGAACA | |
| | | TGT | |
| STAT3 | NM_003150 | TCACATGCCACTTTGGTGTTTCATAA | 1437 |
| | | TCTCCTGGGAGAGATTGACCAGCAGT | |
| | | ATAGCCGCTTCCTGCAAG | |
| STK10 | NM_005990 | CAAGAGGGACTCGGACTGCAGCAGCC | 1438 |
| | | TCTGCACCTCTGAGAGCATGGACTAT | |
| | | GGTACCAATCTCTCCACTGACCTG | |
| STK11 | NM_000455 | GGACTCGGAGACGCTGTGCAGGAGGG | 1439 |
| | | CCGTCAAGATCCTCAAGAAGAAGAAG | |
| | | TTGCGAAGGATCCC | |
| STK15 | NM_003600 | CATCTTCCAGGAGGACCACTCTCTGT | 1440 |
| | | GGCACCCTGGACTACCTGCCCCCTGA | |
| | | AATGATTGAAGGTCGGA | |
| STMN1 | NM_005563 | AATACCCAACGCACAAATGACCGCAC | 1441 |
| | | GTTCTCTGCCCCGTTTCTTGCCCCAG | |
| | | TGTGGTTTGCATTGTCTCC | |
| STMY3 | NM_005940 | CCTGGAGGCTGCAACATACCTCAATC | 1442 |
| | | CTGTCCCAGGCCGGATCCTCCTGAAG | |
| | | CCCTTTTCGCAGCACTGCTATCCTCC | |
| | | AAAGCCATTGTA | |
| SURV | NM_001168 | TGTTTTGATTCCCGGGCTTACCAGGT | 1443 |
| | | GAGAAGTGAGGGAGGAAGAAGGCAGT | |
| | | GTCCCTTTTGCTAGAGCTGACAGCTT | |
| | | TG | |
| TACC3 | NM_006342 | CACCCTTGGACTGGAAAACTCACACC | 1444 |
| | | CGGTCTGGACACAGAAAGAGAACCAA | |
| | | CAGCTCATCAAGG | |
| TBCA | NM_004607 | GATCCTCGCGTGAGACAGATCAAGAT | 1445 |
| | | CAAGACCGGCGTGGTGAAGCGGTTGG | |
| | | TCAAAGAAAAAGTG | |
| TBCC | NM_003192 | CTGTTTTCCTGGAGGACTGCAGTGAC | 1446 |
| | | TGCGTGCTGGCAGTGGCCTGCCAACA | |
| | | GCTCCGCATACACAGT | |
| TBCD | NM_005993 | CAGCCAGGTGTACGAGACATTGCTCA | 1447 |
| | | CCTACAGTGACGTCGTGGGCGCGGAT | |
| | | GTGCTGGACGAGGT | |
| TBCE | NM_003193 | TCCCGAGAGAGGAAAGCATGATGGGA | 1448 |
| | | GCCACGAAGGGACTGTGTATTTTAAA | |
| | | TGCAGGCACCCGAC | |
| TBD | NM_016261 | CCTGGTTGAAGCCTGTTAATGCTTTC | 1449 |
| | | AACGTGTGGAAAACCCAGCGGGCCTT | |
| | | TAGCAAATATGAGAAGTCTGCA | |
| TCP1 | NM_030752 | CCAGTGTGTGTAACAGGGTCACAAGA | 1450 |
| | | ATTCGACAGCCAGATGCTCCAAGAGG | |
| | | GTGGCCCAAGGCTATA | |
| TFRC | NM_003234 | GCCAACTGCTTTCATTTGTGAGGGAT | 1451 |
| | | CTGAACCAATACAGAGCAGACATAAA | |
| | | GGAAATGGGCCTGAGT | |
| THBS1 | NM_003246 | CATCCGCAAAGTGACTGAAGAGAACA | 1452 |
| | | AAGAGTTGGCCAATGAGCTGAGGCGG | |
| | | CCTCCCCTATGCTATCACAACGGAGT | |
| | | TCAGTAC | |
| TK1 | NM_003258 | GCCGGGAAGACCGTAATTGTGGCTGC | 1453 |
| | | ACTGGATGGGACCTTCCAGAGGAAGC | |
| | | CATTTGGGGCCATCCTGAACCTGGTG | |
| | | CCGCTG | |
| TOP2A | NM_001067 | AATCCAAGGGGGAGAGTGATGACTTC | 1454 |
| | | CATATGGACTTTGACTCAGCTGTGGC | |
| | | TCCTCGGGCAAAATCTGTAC | |
| TOP3B | NM_003935 | GTGATGCCTTCCCTGTGGGCGAGGTG | 1455 |
| | | AAGATGCTGGAGAAGCAGACGAACCC | |
| | | ACCCGACTACCTGA | |
| TP | NM_001953 | CTATATGCAGCCAGAGATGTGACAGC | 1456 |
| | | CACCGTGGACAGCCTGCCACTCATCA | |
| | | CAGCCTCCATTCTCAGTAAGAAACTC | |
| | | GTGG | |
| TP53BP1 | NM_005657 | TGCTGTTGCTGAGTCTGTTGCCAGTC | 1457 |
| | | CCCAGAAGACCATGTCTGTGTTGAGC | |
| | | TGTATCTGTGAAGCCAGGCAAG | |
| TPT1 | NM_003295 | GGTGTCGATATTGTCATGAACCATCA | 1458 |
| | | CCTGCAGGAAACAAGTTTCACAAAAG | |
| | | AAGCCTACAAGAAGTACATCAAAGAT | |
| | | TAC | |
| TRAG3 | NM_004909 | GACGCTGGTCTGGTGAAGATGTCCAG | 1459 |
| | | GAAACCACGAGCCTCCAGCCCATTGT | |
| | | CCAACAACCACCCA | |
| TRAIL | NM_003810 | CTTCACAGTGCTCCTGCAGTCTCTCT | 1460 |
| | | GTGTGGCTGTAACTTACGTGTACTTT | |
| | | ACCAACGAGCTGAAGCAGATG | |
| TS | NM_001071 | GCCTCGGTGTGCCTTTCAACATCGCC | 1461 |
| | | AGCTACGCCCTGCTCACGTACATGAT | |
| | | TGCGCACATCACG | |
| TSPAN4 | NM_003271 | CTGGTCAGCCTTCAGGGACCCTGAGC | 1462 |
| | | ACCGCCTGGTCTCTTTCCTGTGGCCA | |
| | | GCCCAGAACTGAAG | |
| TTK | NM_003318 | TGCTTGTCAGTTGTCAACACCTTATG | 1463 |
| | | GCCAACCTGCCTGTTTCCAGCAGCAA | |
| | | CAGCATCAAATACTTGCCACTCCA | |
| TUBA1 | NM_006000 | TGTCACCCCGACTCAACGTGAGACGC | 1464 |
| | | ACCGCCCGGACTCACCATGCGTGAAT | |
| | | GCATCTCAGTCCACGT | |
| TUBA2 | NM_006001 | AGCTCAACATGCGTGAGTGTATCTCT | 1465 |
| | | ATCCACGTGGGGCAGGCAGGAGTCCA | |
| | | GATCGGCAAT | |
| TUBA3 | NM_006009 | CTCTTACATCGACCGCCTAAGAGTCG | 1466 |
| | | CGCTGTAAGAAGCAACAACCTCTCCT | |
| | | CTTCGTCTCCGCCATCAGC | |
| TUBA4 | NM_025019 | GAGGAGGGTGAGTTCTCCAAGGCCCA | 1467 |
| | | TGAGGATATGACTGCCCTGGAGAAGG | |
| | | ATTACAAGGAGGTGGGCAT | |
| TUBA6 | NM_032704 | GTCCCTTCGCCTCCTTCACCGCCGCA | 1468 |
| | | GACCCCTTCAAGTTCTAGTCATGCGT | |
| | | GAGTGCATCTCCATCCACG | |
| TUBA8 | NM_018943 | CGCCCTACCTATACCAACCTCAACCG | 1469 |
| | | CCTCATCAGTCAGATTGTGTCCTCAA | |
| | | TCACTGCTTCTCTCCG | |
| TUBB | NM_001069 | CGAGGACGAGGCTTAAAAACTTCTCA | 1470 |
| | | GATCAATCGTGCATCCTTAGTGAACT | |
| | | TCTGTTGTCCTCAAGCATGGT | |
| TUBB classIII | NM_006086 | CGCCCTCCTGCAGTATTTATGGCCTC | 1471 |
| | | GTCCTCCCCCACCTAGGCCACGTGTG | |
| | | AGCTGCTCCTGTCTCTGT | |
| TUBB1 | NM_030773 | ACACTGACTGGCATCCTGCTTTCCAG | 1472 |
| | | TGCCTGCCAGCCTCCAGAAGAGCCAG | |
| | | GTGCCTGACTAGTACATGGGGAGCTA | |
| | | CAGAGC | |
| TUBB2 | NM_006088 | GTGGCCTAGAGCCTTCAGTCACTGGG | 1473 |
| | | GAAAGCAGGGAAGCAGTGTGAACTCT | |
| | | TTATTCACTCCCAGCCTG | |
| TUBB5 | NM_006087 | ACAGGCCCCATGCATCCTCCCTGCCT | 1474 |
| | | CACTCCCCTCAGCCCCTGCCGACCTT | |
| | | AGCTTATCTGGGAGAGAAACA | |
| TUBBM | NM_032525 | CCCTATGGCCCTGAATGGTGCACTGG | 1475 |
| | | TTTAATTGTGTTGGTGTCGGCCCCTC | |
| | | ACAAATGCAGCCAAGTCATGTAATTA | |
| | | GT | |
| TUBBOK | NM_178014 | AGTGGAATCCTTCCCTTTCCAACTCT | 1476 |
| | | ACCTCCCTCACTCAGCTCCTTTCCCC | |
| | | TGATCAGAGAAAGGGATCAAGGG | |
| TUBBP | NM_178012 | GGAAGGAAAGAAGCATGGTCTACTTT | 1477 |
| | | AGGTGTGCGCTGGGTCTCTGGTGCTC | |
| | | TTCACTGTTGCCTGTCACTTTTT | |
| TUBG1 | NM_001070 | GATGCCGAGGGAAATCATCACCCTAC | 1478 |
| | | AGTTGGGCCAGTGCGGCAATCAGATT | |
| | | GGGTTCGAGTTCTGG | |
| TWIST1 | NM_000474 | GCGCTGCGGAAGATCATCCCCACGCT | 1479 |
| | | GCCCTCGGACAAGCTGAGCAAGATTC | |
| | | AGACCCTCAAGC | |
| TYRO3 | NM_006293 | CAGTGTGGAGGGGATGGAGGAGCCTG | 1480 |
| | | ACATCCAGTGGGTGAAGGATGGGGCT | |
| | | GTGGTCCAGAACTTG | |
| UFM1 | NM_016617 | AGTTGTCGTGTGTTCTGGATTCATTC | 1481 |
| | | CGGCACCACCATGTCGAAGGTTTCCT | |
| | | TTAAGATCACGCTGACG | |
| upa | NM_002658 | GTGGATGTGCCCTGAAGGACAAGCCA | 1482 |
| | | GGCGTCTACACGAGAGTCTCACACTT | |
| | | CTTACCCTGGATCCGCAG | |
| VCAM1 | NM_001078 | TGGCTTCAGGAGCTGAATACCCTCCC | 1483 |
| | | AGGCACACACAGGTGGGACACAAATA | |
| | | AGGGTTTTGGAACCACTATTTTCTCA | |
| | | TCACGACAGCA | |
| VEGF | NM_003376 | CTGCTGTCTTGGGTGCATTGGAGCCT | 1484 |
| | | TGCCTTGCTGCTCTACCTCCACCATG | |
| | | CCAAGTGGTCCCAGGCTGC | |
| VEGFB | NM_003377 | TGACGATGGCCTGGAGTGTGTGCCCA | 1485 |
| | | CTGGGCAGCACCAAGTCCGGATGCAG | |
| | | ATCCTCATGATCCGGTACC | |
| VEGFC | NM_005429 | CCTCAGCAAGACGTTATTTGAAATTA | 1486 |
| | | CAGTGCCTCTCTCTCAAGGCCCCAAA | |
| | | CCAGTAACAATCAGTTTTGCCAATCA | |
| | | CACTT | |
| VHL | NM_000551 | CGGTTGGTGACTTGTCTGCCTCCTGC | 1487 |
| | | TTTGGGAAGACTGAGGCATCCGTGAG | |
| | | GCAGGGACAAGTCTT | |
| VIM | NM_003380 | TGCCCTTAAAGGAACCAATGAGTCCC | 1488 |
| | | TGGAACGCCAGATGCGTGAAATGGAA | |
| | | GAGAACTTTGCCGTTGAAGC | |
| V-RAF | NM_001654 | GGTTGTGCTCTACGAGCTTATGACTG | 1489 |
| | | GCTCACTGCCTTACAGCCACATTGGC | |
| | | TGCCGTGACCAGATTATCTTTATGGT | |
| | | GGGCCG | |
| WAVE3 | NM_006646 | CTCTCCAGTGTGGGCACCAGCCGGCC | 1490 |
| | | AGAACAGATGCGAGCAGTCCATGACT | |
| | | CTGGGAGCTACACCGC | |
| Wnt-5a | NM_003392 | GTATCAGGACCACATGCAGTACATCG | 1491 |
| | | GAGAAGGCGCGAAGACAGGCATCAAA | |
| | | GAATGCCAGTATCAATTCCGACA | |
| XIAP | NM_001167 | GCAGTTGGAAGACACAGGAAAGTATC | 1492 |
| | | CCCAAATTGCAGATTTATCAACGGCT | |
| | | TTTATCTTGAAAATAGTGCCACGCA | |
| XIST | NM_001564 | CAGGTCAGGCAGAGGAAGTCATGTGC | 1493 |
| | | ATTGCATGAGCTAAACCTATCTGAAT | |
| | | GAATTGATTTGGGGCTTGTTAGG | |
| ZW10 | NM_004724 | TGGTCAGATGCTGCTGAAGTATATCC | 1494 |
| | | TTAGGCCGCTGGCATCTTGCCCATCC | |
| | | CTTCATGCTGTGAT | |
| ZWILCH | NM_017975 | GAGGGAGCAGACAGTGGGTACCACGA | 1495 |
| | | TCTCCGTAACCATTTGCATGTGACTT | |
| | | AGCAAGGGCTCTGA | |
| ZWINT | NM_007057 | TAGAGGCCATCAAAATTGGCCTCACC | 1496 |
| | | AAGGCCCTGACTCAGATGGAGGAAGC | |
| | | CCAGAGGAAACGGA | |

TABLE 1

| | | | Estimated |
|---|---|---|---|
| | Gene | p-value | Coefficient |
| 1 | SLC1A3 | 0.0002 | −0.7577 |
| 2 | TBCC | 0.0006 | −1.0289 |
| 3 | EIF4E2 | 0.0009 | −1.2038 |
| 4 | TUBB | 0.0017 | −0.7332 |
| 5 | TSPAN4 | 0.0027 | −0.7211 |
| 6 | VHL | 0.0034 | −0.7450 |
| 7 | BAX | 0.0039 | −1.0224 |
| 8 | CD247 | 0.0044 | −0.4656 |
| 9 | CAPZA1 | 0.0044 | −1.1182 |
| 10 | STMN1 | 0.0052 | −0.4350 |
| 11 | ABCC1 | 0.0054 | −0.7653 |
| 12 | ZW10 | 0.0055 | −0.8228 |
| 13 | HSPA1B | 0.0058 | −0.4740 |
| 14 | MAPRE1 | 0.0060 | −0.7833 |
| 15 | PLD3 | 0.0061 | −0.8595 |
| 16 | APRT | 0.0062 | −0.7714 |
| 17 | BAK1 | 0.0064 | −0.7515 |
| 18 | TUBA6 | 0.0067 | −0.7006 |
| 19 | CST7 | 0.0069 | −0.4243 |
| 20 | SHC1 | 0.0080 | −0.6632 |
| 21 | ZWILCH | 0.0088 | −0.6902 |
| 22 | SRC | 0.0089 | −0.7011 |
| 23 | GADD45B | 0.0102 | −0.5253 |
| 24 | LIMK2 | 0.0106 | −0.7784 |
| 25 | CENPA | 0.0106 | −0.3588 |
| 26 | CHEK2 | 0.0109 | −0.6737 |
| 27 | RAD1 | 0.0115 | −0.6673 |
| 28 | MRE11A | 0.0120 | −0.6253 |
| 29 | DDR1 | 0.0122 | −0.5660 |
| 30 | STK10 | 0.0123 | −0.6002 |
| 31 | LILRB1 | 0.0125 | −0.4674 |
| 32 | BBC3 | 0.0128 | −0.4481 |
| 33 | BUB3 | 0.0144 | −0.5476 |
| 34 | CDCA8 | 0.0145 | −0.3759 |
| 35 | TOP3B | 0.0164 | −0.7292 |
| 36 | RPN2 | 0.0166 | −0.8121 |
| 37 | ILK | 0.0169 | −0.6920 |
| 38 | GBP1 | 0.0170 | −0.3496 |
| 39 | TUBB3 | 0.0173 | −0.3037 |
| 40 | NTSR2 | 0.0175 | −2.4355 |
| 41 | BID | 0.0175 | −0.6228 |
| 42 | BCL2L13 | 0.0189 | −0.7228 |
| 43 | TPX2 | 0.0196 | −0.3148 |
| 44 | ABCC5 | 0.0203 | −0.3906 |
| 45 | HDAC6 | 0.0226 | −0.7782 |
| 46 | CD68 | 0.0226 | −0.6531 |
| 47 | NEK2 | 0.0232 | −0.3657 |
| 48 | DICER1 | 0.0233 | −0.5537 |
| 49 | RHOA | 0.0268 | −0.7407 |
| 50 | TYMS | 0.0291 | −0.3577 |
| 51 | CCT3 | 0.0292 | −0.5989 |
| 52 | ACTR2 | 0.0297 | −0.8754 |
| 53 | WNT5A | 0.0321 | 0.5036 |
| 54 | HSPA1L | 0.0321 | −1.8702 |
| 55 | APOC1 | 0.0324 | −0.3434 |
| 56 | ZWINT | 0.0326 | −0.3966 |
| 57 | APEX1 | 0.0330 | −0.7200 |
| 58 | KALPHA1 | 0.0351 | −0.7627 |
| 59 | ABCC10 | 0.0354 | −0.5667 |
| 60 | PHB | 0.0380 | −0.5832 |
| 61 | TUBB2C | 0.0380 | −0.6664 |
| 62 | RALBP1 | 0.0382 | −0.5989 |
| 63 | VEGF | 0.0397 | −0.3673 |
| 64 | MCL1 | 0.0398 | −0.6137 |
| 65 | HSPA1A | 0.0402 | −0.3451 |
| 66 | BUB1 | 0.0404 | −0.2911 |
| 67 | MAD2L1 | 0.0412 | −0.3336 |
| 68 | CENPF | 0.0418 | −0.2979 |
| 69 | IL2RA | 0.0427 | −0.5023 |
| 70 | TUBA3 | 0.0429 | −0.4528 |
| 71 | ACTB | 0.0439 | −0.8259 |
| 72 | KIF22 | 0.0447 | −0.5427 |
| 73 | CXCR4 | 0.0462 | −0.4239 |
| 74 | STAT1 | 0.0472 | −0.3555 |
| 75 | IL7 | 0.0473 | −0.3973 |
| 76 | CHFR | 0.0499 | −0.5387 |

TABLE 2

| | | | Estimated |
|---|---|---|---|
| | Gene | p-value | Coefficient |
| 1 | DDR1 | <.0001 | −1.2307 |
| 2 | EIF4E2 | 0.0001 | −1.8076 |
| 3 | TBCC | 0.0001 | −1.5303 |
| 4 | STK10 | 0.0005 | −1.2320 |
| 5 | ZW10 | 0.0006 | −1.3917 |
| 6 | BBC3 | 0.0010 | −0.9034 |
| 7 | BAX | 0.0011 | −1.4992 |
| 8 | BAK1 | 0.0011 | −1.3122 |
| 9 | TSPAN4 | 0.0013 | −1.1930 |
| 10 | SLC1A3 | 0.0014 | −0.9828 |
| 11 | SHC1 | 0.0015 | −1.1395 |
| 12 | CHFR | 0.0016 | −1.3371 |
| 13 | RHOB | 0.0018 | −0.7059 |
| 14 | TUBA6 | 0.0019 | −1.1071 |
| 15 | BCL2L13 | 0.0023 | −1.3181 |
| 16 | MAPRE1 | 0.0029 | −1.2233 |
| 17 | GADD45B | 0.0034 | −0.9174 |
| 18 | HSPA1B | 0.0036 | −0.6406 |
| 19 | FAS | 0.0037 | −0.8571 |
| 20 | TUBB | 0.0040 | −1.0178 |
| 21 | HSPA1A | 0.0041 | −0.6648 |
| 22 | MCL1 | 0.0041 | −1.1459 |
| 23 | CCT3 | 0.0048 | −1.0709 |
| 24 | VEGF | 0.0049 | −0.8411 |
| 25 | TUBB2C | 0.0051 | −1.4181 |
| 26 | AKT1 | 0.0053 | −1.1175 |
| 27 | MAD2L1BP | 0.0055 | −1.0691 |
| 28 | RPN2 | 0.0056 | −1.2688 |
| 29 | RHOA | 0.0063 | −1.3773 |
| 30 | MAP2K3 | 0.0063 | −0.9616 |
| 31 | BID | 0.0067 | −1.0502 |
| 32 | APOE | 0.0074 | −0.8130 |
| 33 | ESR1 | 0.0077 | −0.3456 |
| 34 | ILK | 0.0084 | −1.1481 |
| 35 | NTSR2 | 0.0090 | −4.0522 |
| 36 | TOP3B | 0.0091 | −1.0744 |
| 37 | PLD3 | 0.0095 | −1.1126 |
| 38 | DICER1 | 0.0095 | −0.8849 |
| 39 | VHL | 0.0104 | −0.9357 |
| 40 | GCLC | 0.0108 | −0.7822 |
| 41 | RAD1 | 0.0108 | −1.0141 |
| 42 | GATA3 | 0.0112 | −0.4400 |
| 43 | CXCR4 | 0.0120 | −0.7032 |
| 44 | NME6 | 0.0121 | −0.9873 |
| 45 | UFM1 | 0.0125 | −0.9686 |
| 46 | BUB3 | 0.0126 | −0.9054 |
| 47 | CD14 | 0.0130 | −0.8152 |
| 48 | MRE11A | 0.0130 | −0.8915 |
| 49 | CST7 | 0.0131 | −0.5204 |
| 50 | APOC1 | 0.0134 | −0.5630 |
| 51 | GNS | 0.0136 | −1.0979 |
| 52 | ABCC5 | 0.0146 | −0.5595 |
| 53 | AKT2 | 0.0150 | −1.0824 |
| 54 | APRT | 0.0150 | −0.9231 |
| 55 | PLAU | 0.0157 | −0.6705 |
| 56 | RCC1 | 0.0163 | −0.9073 |
| 57 | CAPZA1 | 0.0165 | −1.3542 |
| 58 | RELA | 0.0168 | −0.8534 |
| 59 | NFKB1 | 0.0179 | −0.9847 |
| 60 | RASSF1 | 0.0186 | −0.8078 |
| 61 | BCL2L11 | 0.0209 | −0.9394 |
| 62 | CSNK1D | 0.0211 | −1.2276 |
| 63 | SRC | 0.0220 | −0.8341 |
| 64 | LIMK2 | 0.0221 | −1.0830 |
| 65 | SIRT1 | 0.0229 | −0.7236 |
| 66 | RXRA | 0.0247 | −0.7973 |
| 67 | ABCD1 | 0.0259 | −0.7533 |
| 68 | MAPK3 | 0.0269 | −0.7322 |
| 69 | CDCA8 | 0.0275 | −0.5210 |
| 70 | DUSP1 | 0.0284 | −0.3398 |
| 71 | ABCC1 | 0.0287 | −0.8003 |
| 72 | PRKCH | 0.0291 | −0.6680 |
| 73 | PRDX1 | 0.0301 | −0.8823 |
| 74 | TUBA3 | 0.0306 | −0.7331 |
| 75 | VEGFB | 0.0317 | −0.7487 |
| 76 | LILRB1 | 0.0320 | −0.5617 |
| 77 | LAPTM4B | 0.0321 | −0.4994 |
| 78 | HSPA9B | 0.0324 | −0.9660 |
| 79 | ECGF1 | 0.0329 | −0.5807 |
| 80 | GDF15 | 0.0332 | −0.3646 |
| 81 | ACTR2 | 0.0347 | −1.1827 |
| 82 | IL7 | 0.0349 | −0.5623 |
| 83 | HDAC6 | 0.0380 | −0.9486 |
| 84 | ZWILCH | 0.0384 | −0.7296 |
| 85 | CHEK2 | 0.0392 | −0.7502 |
| 86 | REG1A | 0.0398 | −3.4734 |
| 87 | APC | 0.0411 | −0.8324 |
| 88 | SLC35B1 | 0.0411 | −0.6801 |
| 89 | NEK2 | 0.0415 | −0.4609 |
| 90 | ACTB | 0.0418 | −1.1482 |
| 91 | BUB1 | 0.0423 | −0.4612 |
| 92 | PPP2CA | 0.0423 | −0.9474 |
| 93 | TNFRSF10A | 0.0448 | −0.6415 |
| 94 | TBCD | 0.0456 | −0.6196 |
| 95 | ERBB4 | 0.0460 | −0.2830 |
| 96 | CDC25B | 0.0467 | −0.5660 |
| 97 | STMN1 | 0.0472 | −0.4684 |

TABLE 3

| | | | Estimated |
|---|---|---|---|
| | Gene | p-value | Coefficient |
| 1 | DDR1 | <.0001 | −1.3498 |
| 2 | ZW10 | <.0001 | −2.1657 |
| 3 | RELA | <.0001 | −1.5759 |
| 4 | BAX | <.0001 | −1.8857 |
| 5 | RHOB | <.0001 | −1.1694 |
| 6 | TSPAN4 | <.0001 | −1.7067 |
| 7 | BBC3 | <.0001 | −1.2017 |
| 8 | SHC1 | <.0001 | −1.4625 |
| 9 | CAPZA1 | <.0001 | −2.4068 |
| 10 | STK10 | 0.0001 | −1.4013 |
| 11 | TBCC | 0.0001 | −1.6385 |
| 12 | EIF4E2 | 0.0002 | −1.9122 |
| 13 | MCL1 | 0.0003 | −1.6617 |
| 14 | RASSF1 | 0.0003 | −1.3201 |
| 15 | VEGF | 0.0003 | −1.0800 |
| 16 | SLC1A3 | 0.0004 | −1.0855 |
| 17 | DICER1 | 0.0004 | −1.4236 |
| 18 | ILK | 0.0004 | −1.7221 |
| 19 | FAS | 0.0005 | −1.1671 |
| 20 | RAB6C | 0.0005 | −1.6154 |
| 21 | ESR1 | 0.0006 | −0.4845 |
| 22 | MRE11A | 0.0006 | −1.2537 |
| 23 | APOE | 0.0006 | −1.0602 |
| 24 | BAK1 | 0.0006 | −1.4288 |
| 25 | UFM1 | 0.0006 | −1.4110 |
| 26 | AKT2 | 0.0007 | −1.6213 |
| 27 | SIRT1 | 0.0007 | −1.1651 |
| 28 | BCL2L13 | 0.0008 | −1.5059 |
| 29 | ACTR2 | 0.0008 | −1.9690 |
| 30 | LIMK2 | 0.0009 | −1.6937 |
| 31 | HDAC6 | 0.0010 | −1.5715 |
| 32 | RPN2 | 0.0010 | −1.5839 |
| 33 | PLD3 | 0.0010 | −1.5460 |
| 34 | CHGA | 0.0011 | −0.8275 |
| 35 | RHOA | 0.0011 | −1.6934 |
| 36 | MAPK14 | 0.0014 | −1.6611 |
| 37 | ECGF1 | 0.0014 | −0.8835 |
| 38 | MAPRE1 | 0.0016 | −1.3329 |
| 39 | HSPA1B | 0.0017 | −0.8048 |
| 40 | GATA3 | 0.0017 | −0.6153 |
| 41 | PPP2CA | 0.0017 | −1.6176 |
| 42 | ABCD1 | 0.0018 | −1.1669 |
| 43 | MAD2L1BP | 0.0018 | −1.1725 |
| 44 | VHL | 0.0022 | −1.1855 |
| 45 | GCLC | 0.0023 | −1.1240 |
| 46 | ACTB | 0.0023 | −1.8754 |
| 47 | BCL2L11 | 0.0024 | −1.5415 |
| 48 | PRDX1 | 0.0025 | −1.3943 |
| 49 | LILRB1 | 0.0025 | −0.8462 |
| 50 | GNS | 0.0025 | −1.3307 |
| 51 | CHFR | 0.0026 | −1.3292 |
| 52 | CD68 | 0.0026 | −1.1941 |
| 53 | LIMK1 | 0.0026 | −1.5655 |
| 54 | GADD45B | 0.0027 | −1.0162 |
| 55 | VEGFB | 0.0027 | −1.1252 |
| 56 | APRT | 0.0027 | −1.2629 |
| 57 | MAP2K3 | 0.0031 | −1.1297 |
| 58 | MGC52057 | 0.0033 | −1.0906 |
| 59 | MAPK3 | 0.0033 | −1.0390 |
| 60 | APC | 0.0034 | −1.2719 |
| 61 | RAD1 | 0.0036 | −1.2744 |
| 62 | COL6A3 | 0.0039 | −0.8240 |
| 63 | RXRB | 0.0039 | −1.2638 |
| 64 | CCT3 | 0.0040 | −1.3329 |
| 65 | ABCC3 | 0.0040 | −0.8170 |
| 66 | GPX1 | 0.0042 | −1.5547 |
| 67 | TUBB2C | 0.0042 | −1.6184 |
| 68 | HSPA1A | 0.0043 | −0.7875 |
| 69 | AKT1 | 0.0045 | −1.1777 |
| 70 | TUBA6 | 0.0046 | −1.2048 |
| 71 | TOP3B | 0.0048 | −1.1950 |
| 72 | CSNK1D | 0.0049 | −1.6201 |
| 73 | SOD1 | 0.0049 | −1.2383 |
| 74 | BUB3 | 0.0050 | −1.0111 |
| 75 | MAP4 | 0.0052 | −1.5220 |
| 76 | NFKB1 | 0.0060 | −1.2355 |
| 77 | SEC61A1 | 0.0060 | −1.4777 |
| 78 | MAD1L1 | 0.0060 | −1.1168 |
| 79 | PRKCH | 0.0073 | −0.8259 |
| 80 | RXRA | 0.0074 | −0.9693 |
| 81 | PLAU | 0.0074 | −0.7987 |
| 82 | CD63 | 0.0074 | −1.3830 |
| 83 | CD14 | 0.0075 | −0.9409 |
| 84 | RHOC | 0.0077 | −1.0341 |
| 85 | STAT1 | 0.0093 | −0.7663 |
| 86 | NPC2 | 0.0094 | −1.2302 |
| 87 | NME6 | 0.0095 | −1.2091 |
| 88 | PDGFRB | 0.0096 | −0.7932 |
| 89 | MGMT | 0.0098 | −1.0325 |
| 90 | GBP1 | 0.0098 | −0.5896 |
| 91 | ERCC1 | 0.0105 | −1.2240 |
| 92 | RCC1 | 0.0107 | −1.0453 |
| 93 | FUS | 0.0117 | −1.2869 |
| 94 | TUBA3 | 0.0117 | −0.8905 |
| 95 | CHEK2 | 0.0120 | −1.0057 |
| 96 | APOC1 | 0.0123 | −0.6422 |
| 97 | ABCC10 | 0.0124 | −0.9400 |
| 98 | SRC | 0.0128 | −1.1170 |
| 99 | TUBB | 0.0136 | −0.9398 |
| 100 | FLAD1 | 0.0139 | −1.0396 |
| 101 | MAD2L2 | 0.0141 | −1.0834 |
| 102 | LAPTM4B | 0.0149 | −0.5932 |
| 103 | REG1A | 0.0150 | −5.1214 |
| 104 | PRKCD | 0.0152 | −1.0120 |
| 105 | CST7 | 0.0157 | −0.5499 |
| 106 | IGFBP2 | 0.0161 | −0.5019 |
| 107 | FYN | 0.0162 | −0.7670 |
| 108 | KDR | 0.0168 | −0.8204 |
| 109 | STMN1 | 0.0169 | −0.6791 |
| 110 | ZWILCH | 0.0170 | −0.8897 |
| 111 | RBM17 | 0.0171 | −1.3981 |
| 112 | TP53BP1 | 0.0184 | −0.9442 |
| 113 | CD247 | 0.0188 | −0.5768 |
| 114 | ABCA9 | 0.0190 | −0.5489 |
| 115 | NTSR2 | 0.0192 | −3.9043 |
| 116 | FOS | 0.0195 | −0.4437 |
| 117 | TNFRSF10A | 0.0196 | −0.7666 |
| 118 | MSH3 | 0.0200 | −0.9585 |
| 119 | PTEN | 0.0202 | −1.0307 |
| 120 | GBP2 | 0.0204 | −0.6414 |
| 121 | STK11 | 0.0206 | −0.9807 |
| 122 | ERBB4 | 0.0213 | −0.3933 |
| 123 | TFF1 | 0.0220 | −0.2020 |
| 124 | ABCC1 | 0.0222 | −0.9438 |
| 125 | IL7 | 0.0223 | −0.6920 |
| 126 | CDC25B | 0.0228 | −0.7338 |
| 127 | TUBD1 | 0.0234 | −0.6092 |
| 128 | BIRC4 | 0.0236 | −0.9072 |
| 129 | ACTR3 | 0.0246 | −1.3384 |
| 130 | SLC35B1 | 0.0253 | −0.7793 |
| 131 | COL1A1 | 0.0256 | −0.4945 |
| 132 | FOXA1 | 0.0262 | −0.4554 |
| 133 | DUSP1 | 0.0264 | −0.4205 |
| 134 | CXCR4 | 0.0265 | −0.6550 |
| 135 | IL2RA | 0.0268 | −0.9731 |
| 136 | GGPS1 | 0.0268 | −0.7915 |
| 137 | KNS2 | 0.0281 | −0.8758 |
| 138 | RB1 | 0.0289 | −0.9291 |
| 139 | BCL2L1 | 0.0289 | −0.9123 |
| 140 | XIST | 0.0294 | −0.6529 |
| 141 | BIRC3 | 0.0294 | −0.4739 |
| 142 | BID | 0.0303 | −0.8691 |
| 143 | BCL2 | 0.0303 | −0.5525 |
| 144 | STAT3 | 0.0311 | −0.9289 |
| 145 | PECAM1 | 0.0319 | −0.6803 |
| 146 | DIABLO | 0.0328 | −0.9572 |
| 147 | CYBA | 0.0333 | −0.6642 |
| 148 | TBCE | 0.0336 | −0.7411 |
| 149 | CYP1B1 | 0.0337 | −0.6013 |
| 150 | APEX1 | 0.0357 | −1.0916 |
| 151 | TBCD | 0.0383 | −0.5893 |
| 152 | HRAS | 0.0390 | −0.8411 |
| 153 | TNFRSF10B | 0.0394 | −0.7293 |
| 154 | ELP3 | 0.0398 | −0.9560 |
| 155 | PIK3C2A | 0.0408 | −0.9158 |
| 156 | HSPA5 | 0.0417 | −1.5232 |
| 157 | VEGFC | 0.0427 | −0.7309 |
| 158 | CRABP1 | 0.0440 | −0.2492 |
| 159 | MMP11 | 0.0456 | −0.3894 |
| 160 | SGK | 0.0456 | −0.6740 |
| 161 | CTSD | 0.0463 | −0.7166 |
| 162 | BAD | 0.0479 | −0.6436 |
| 163 | PTPN21 | 0.0484 | −0.5636 |
| 164 | HSPA9B | 0.0487 | −0.9657 |
| 165 | PMS1 | 0.0498 | −0.9283 |

TABLE 4

| | | | Estimated |
|---|---|---|---|
| | Gene | p-value | Coefficient |
| 1 | CD247 | 0.0101 | −0.6642 |
| 2 | TYMS | 0.0225 | −0.5949 |
| 3 | IGF1R | 0.0270 | −0.5243 |
| 4 | ACTG2 | 0.0280 | −0.2775 |
| 5 | CCND1 | 0.0355 | 0.4802 |
| 6 | CAPZA1 | 0.0401 | −1.1408 |
| 7 | CHEK2 | 0.0438 | −0.9595 |
| 8 | STMN1 | 0.0441 | −0.5369 |
| 9 | ZWILCH | 0.0476 | −0.8264 |

TABLE 5

| Official | | | |
|---|---|---|---|
| Symbol | Name | Entrez | Role |
| CHUK | Conserved helix-loop-helix ubiquitous kinase | 1147 | Activates |
| BCL3 | B-cell CLL/lymphoma 3 | 602 | Transcriptional co-activator |
| FADD | Fas (TNFRSF6)-associated via death domain | 8772 | Stimulates pathway |
| IKBKB | Inhibitor of kappa light polypeptide gene | 3551 | Activates; triggers |
| | enhancer in B-cells, kinase beta | | degradation of NFKBIA, |
| | | | NFKBIB |
| IKBKG | Inhibitor of kappa light polypeptide gene | 8517 | Activates; triggers |
| | enhancer in B-cells, kinase gamma | | degradation of NFKBIA, |
| | | | NFKBIB |
| IL1A | Interleukin 1, alpha | 3552 | Stimulates pathway |
| IL1R1 | Interleukin 1 receptor, type I | 3554 | Stimulates pathway |
| IRAK1 | Interleukin-1 receptor-associated kinase 1 | 3654 | Stimulates pathway |
| NFKB1 | Nuclear factor of kappa light polypeptide gene | 4790 | Core subunit |
| | enhancer in B-cells 1 (p105) | | |
| NFKB2 | Nuclear factor of kappa light polypeptide gene | 4791 | Core subunit |
| | enhancer in B-cells 2 (p49/p100) | | |
| NFKBIA | Nuclear factor of kappa light polypeptide gene | 4792 | Inhibits |
| | enhancer in B-cells inhibitor, alpha | | |
| NFKBIB | Nuclear factor of kappa light polypeptide gene | 4793 | Inhibits |
| | enhancer in B-cells inhibitor, beta | | |
| NFKBIE | nuclear factor of kappa light polypeptide gene | 4794 | Inhibits |
| | enhancer in B-cells inhibitor, epsilon | | |
| REL | v-rel reticuloendotheliosis viral oncogene | 5966 | Transcriptional co-activator |
| | homolog (avian) | | |
| RELA | V-rel reticuloendotheliosis viral oncogene | 5970 | Transcriptional co-activator |
| | homolog A, nuclear factor of kappa light | | |
| | polypeptide gene enhancer in B-cells 3, p65 | | |
| | (avian) | | |
| RELB | v-rel reticuloendotheliosis viral oncogene | 5971 | Transcriptional co-activator |
| | homolog B, nuclear factor of kappa light | | |
| | polypeptide gene enhancer in B-cells 3 | | |
| | (avian) | | |
| RHOC | ras homolog gene family, member C | 389 | Induce activation of pathway |
| TNFAIP3 | Tumor necrosis factor, alpha-induced protein 3 | 7128 | Activates |
| TNFRSF1A | Tumor necrosis factor receptor superfamily, | 7132 | Activates |
| | member 1A | | |
| TNFRSF1B | TNFRSF1A-associated via death domain | 7133 | Activates |
| TRAF6 | TNF receptor-associated factor 6 | 7189 | Activates CHUK |

What is claimed is:

1. A method of predicting whether a hormone receptor (HR) positive cancer patient will exhibit a beneficial response to chemotherapy, comprising
measuring an expression level of a gene, or its expression product, in a tumor sample obtained from the patient, wherein the gene is selected from the group consisting of ABCC1, ABCC5, ABCD1, ACTB, ACTR2, AKT1, AKT2, APC, APOC1, APOE, APRT, BAK1, BAX, BBC3, BCL2 μl, BCL2L13, BID, BUB1, BUB3, CAPZA1, CCT3, CD14, CDC25B, CDCA8, CHEK2, CHFR, CSNK1D, CST7, CXCR4, DDR1, DICER1, DUSP1, ECGF1, EIF4E2, ERBB4, ESR1, FAS, GADD45B, GATA3, GCLC, GDF15, GNS, HDAC6, HSPA1A, HSPA1B, HSPA9B, IL7, ILK, LAPTM4B, LILRB1, LIMK2, MAD2L1BP, MAP2K3, MAPK3, MAPRE1, MCL1, MRE11A, NEK2, NFKB1, NME6, NTSR2, PLAU, PLD3, PPP2CA, PRDX1, PRKCH, RAD1, RASSF1, RCC1, REG1A, RELA, RHOA, RHOB, RPN2, RXRA, SHC1, SIRT1, SLC1A3, SLC35B1, SRC, STK10, STMN1, TBCC, TBCD, TNFRSF10A, TOP3B, TSPAN4, TUBA3, TUBA6, TUBB, TUBB2C, UFM1, VEGF, VEGFB, VHL, ZW10, and ZWILCH;
using the expression level to determine a likelihood of a beneficial response to a treatment including a taxane,
wherein expression of DDR1, EIF4E2, TBCC, STK10, ZW10, BBC3, BAX, BAK1, TSPAN4, SLC1A3, SHC1, CHFR, RHOB, TUBA6, BCL2L13, MAPRE1, GADD45B, HSPA1B, FAS, TUBB, HSPA1A, MCL1, CCT3, VEGF, TUBB2C, AKT1, MAD2L1BP, RPN2, RHOA, MAP2K3, BID, APOE, ESR1, ILK, NTSR2, TOP3B, PLD3, DICER1, VHL, GCLC, RAD1, GATA3, CXCR4, NME6, UFM1, BUB3, CD14, MRE11A, CST7, APOC1, GNS, ABCC5, AKT2, APRT, PLAU, RCC1, CAPZA1, RELA, NFKB1, RASSF1, BCL2L11, CSNK1D, SRC, LIMK2, SIRT1, RXRA, ABCD1, MAPK3, DUSP1, ABCC1, PRKCH, PRDX1, TUBA3, VEGFB, LILRB1, LAPTM4B, HSPA9B, ECGF1, GDF15, ACTR2, IL7, HDAC6, CHEK2, REG1A, APC, SLC35B1, ACTB, PPP2CA, TNFRSF10A, TBCD, ERBB4, CDC25B, or STMN1 is positively correlated with increased likelihood of a beneficial response to a treatment including a taxane, and
wherein expression of CDCA8, ZWILCH, NEK2, or BUB1 is negatively correlated with an increased likelihood of a beneficial response to a treatment including a taxane; and
generating a report including information based on the likelihood of a beneficial response to chemotherapy including a taxane.

2. The method of claim 1, wherein the method comprises using the expression level to determine a likelihood of a beneficial response to a treatment including a cyclophosphamide,
wherein expression of ZW10, BAX, GADD45B, FAS, ESR1, NME6, MRE11A, AKT2, RELA, RASSF1, PRKCH, VEGFB, LILRB1, ACTR2, REG1A, or PPP2CA is positively correlated with increased likelihood of a beneficial response to a treatment including a cyclophosphamide, and
wherein expression of DDR1, EIF4E2, TBCC, STK10, BBC3, BAK1, TSPAN4, SHC1, CHFR, RHOB, TUBA6, BCL2L13, MAPRE1, HSPA1, TUBB, HSPA1A, MCL1, CCT3, VEGF, TUBB2C, AKT1, MAD2L1BP, RPN2, RHOA, MAP2K3, BID, APOE, ILK, NTSR2, TOP3B, PLD3, DICER1, VHL, GCLC, RAD1, GATA3, CXCR4, UFM1, BUB3, CD14, CST7, APOC1, GNS, ABCC5, APRT, PLAU, RCC1, CAPZA1, NFKB1, BCL2L11, CSNK1D, SRC, LIMK2, SIRT1, RXRA, ABCD1, MAPK3, CDCA8, DUSP1, ABCC1, PRDX1, TUBA3, LAPTM4B, HSPA9B, ECGF1, GDF15, IL7, HDAC6, ZWILCH, CHEK2, APC, SLC35B1, NEK2, ACTB, BUB1, TNFRSF10A, TBCD, ERBB4, CDC25B, or STMN1 is negatively correlated with an increased likelihood of a beneficial response to a treatment including a cyclophosphamide, and
wherein the report includes information based on the likelihood of a beneficial response to chemotherapy including a cyclophosphamide.

3. The method of claim 1, wherein the chemotherapy includes an anthracycline.

4. The method of claim 3, wherein the anthracycline is doxorubicin.

5. The method of claim 1, wherein the taxane is docetaxel.

6. The method of claim 1, wherein said measuring is by quantitative PCR.

7. The method of claim 1, wherein said measuring is by detection of an intron-based sequence of an RNA transcript of the gene, wherein the expression of which correlates with the expression of a corresponding exon sequence.

8. The method of claim 1, wherein the tumor sample is a formalin-fixed and paraffin-embedded (FPE) or a frozen tumor section.

9. A method of predicting whether a hormone receptor (HR) positive cancer patient will exhibit a beneficial response to chemotherapy, comprising
measuring an expression level of a gene, or its expression product, in a tumor sample obtained from the patient, wherein the gene is selected from the group consisting of ABCA9, ABCC1, ABCC10, ABCC3, ABCD1, ACTB, ACTR2, ACTR3, AKT1, AKT2, APC, APEX1, APOC1, APOE, APRT, BAD, BAK1, BAX, BBC3, BCL2, BCL2L1, BCL2L11, BCL2L13, BID, BIRC3, BIRC4, BUB3, CAPZA1, CCT3, CD14, CD247, CD63, CD68, CDC25B, CHEK2, CHFR, CHGA, COL1A1, COL6A3, CRABP1, CSNK1D, CST7, CTSD, CXCR4, CYBA, CYP1B1, DDR1, DIABLO, DICER1, DUSP1, ECGF1, EIF4E2, ELP3, ERBB4, ERCC1, ESR1, FAS, FLAD1, FOS, FOXA1, FUS, FYN, GADD45B, GATA3, GBP1, GBP2, GCLC, GGPS1, GNS, GPX1, HDAC6, HRAS, HSPA1A, HSPA1B, HSPA5, HSPA9B, IGFBP2, IL2RA, IL7, ILK, KDR, KNS2, LAPTM4B, LILRB1, LIMK1, LIMK2, MAD1L1, MAD2L1BP, MAD2L2, MAP2K3, MAP4, MAPK14, MAPK3, MAPRE1, MCL1, MGC52057, MGMT, MMP11, MRE11A, MSH3, NFKB1, NME6, NPC2, NTSR2, PDGFRB, PECAM1, PIK3C2A, PLAU, PLD3, PMS1, PPP2CA, PRDX1, PRKCD, PRKCH, PTEN, PTPN21, RAB6C, RAD1, RASSF1, RB1, RBM17, RCC1, REG1A, RELA, RHOA, RHOB, RHOC, RPN2, RXRA, RXRB, SEC61A1, SGK, SHC1, SIRT1, SLC1A3, SLC35B1, SOD1, SRC, STAT1, STAT3, STK10, STK11, STMN1, TBCC, TBCD, TBCE, TFF1, TNFRSF10A, TNFRSF10B, TOP3B, TP53BP1, TSPAN4, TUBA3, TUBA6, TUBB, TUBB2C, TUBD1, UFM1, VEGF, VEGFB, VEGFC, VHL, XIST, ZW10, and ZWILCH;
using the expression level to determine a likelihood of a beneficial response to a treatment including a taxane,
wherein expression of DDR1, ZW10, RELA, BAX, RHOB, TSPAN4, BBC3, SHC1, CAPZA1, STK10, TBCC, EIF4E2, MCL1, RASSF1, VEGF, SLC1A3, DICER1, ILK, FAS, RAB6C, ESR1, MRE11A, APOE, BAK1, UFM1, AKT2, SIRT1, BCL2L13, ACTR2, LIMK2, HDAC6, RPN2, PLD3, RHOA, MAPK14, ECGF1, MAPRE1, HSPA1B, GATA3, PPP2CA, ABCD1, MAD2L1BP, VHL, GCLC, ACTB, BCL2L11, PRDX1, LILRB1, GNS, CHFR, CD68, LIMK1, GADD45B, VEGFB, APRT, MAP2K3, MGC52057, MAPK3, APC, RAD1, COL6A3, RXRB, CCT3, ABCC3, GPX1, TUBB2C, HSPA1A, AKT1, TUBA6, TOP3B, CSNK1D, SOD1, BUB3, MAP4, NFKB1, SEC61A1, MAD1L1, PRKCH, RXRA, PLAU, CD63, CD14, RHOC, STAT1, NPC2, NME6, PDGFRB, MGMT1, GBP1, ERCC1, RCC1, FUS, TUBA3, CHEK2, APOC1, ABCC10, SRC, TUBB, FLAD1, MAD2L2, LAPTM4B, REG1A, PRKCD, CST7, IGFBP2, FYN, KDR, STMN1, RBM17, TP53BP1, CD247, ABCA9, NTSR2, FOS, TNFRSF10A, MSH3, PTEN, GBP2, STK11, ERBB4, TFF1, ABCC1, IL7, CDC25B, TUBD1, BIRC4, ACTR3, SLC35B1, COL1A1, FOXA1, DUSP1, CXCR4, IL2RA, GGPS1, KNS2, RB1, BCL2L1, XIST, BIRC3, BID, BCL2, STAT3, PECAM1, DIABLO, CYBA, TBCE, CYP1B1, APEX1, TBCD, HRAS, TNFRSF10B, ELP3, PIK3C2A, HSPA5, VEGFC, MMP11, SGK, CTSD, BAD, PTPN21, HSPA9B, or PMS1 is positively correlated with increased likelihood of a beneficial response to a treatment including a taxane, and
wherein expression of CHGA, ZWILCH, or CRABP1 is negatively correlated with an increased likelihood of a beneficial response to a treatment including a taxane; and
generating a report including information based on the likelihood of a beneficial response to chemotherapy including a taxane.

10. The method of claim 9, wherein the method comprises using the expression level to determine a likelihood of a beneficial response to a treatment including a cyclophosphamide,
wherein expression of LILRB1, PRKCH, STAT1, GBP1, CD247, IL7, IL2RA, BIRC3, or CRABP1 is positively correlated with increased likelihood of a beneficial response to a treatment including a cyclophosphamide, and
wherein expression of DDR1, ZW10, RELA, BAX, RHOB, TSPAN4, BBC3, SHC1, CAPZA1, STK10, TBCC, EIF4E2, MCL1, RASSF1, VEGF, DICER1, ILK, FAS, RAB6C, ESR1, MRE11A, APOE, BAK1, UFM1, AKT2, SIRT1, BCL2L13, ACTR2, LIMK2, HDAC6, RPN2, PLD3, CHGA, RHOA, MAPK14, ECGF1, MAPRE1, HSPA1B, GATA3, PPP2CA, ABCD1, MAD2L1BP, VHL, GCLC, ACTB, BCL2L11, PRDX1, GNS, CHFR, CD68, LIMK1, GADD45B, VEGFB, APRT, MAP2K3, MGC52057, MAPK3, APC, RAD1, COL6A3, RXRB, CCT3, ABCC3, GPX1, TUBB2C, HSPA1A, AKT1, TUBA6, TOP3B, CSNK1D, SOD1, BUB3, MAP4, NFKB1, SEC61A1, MAD1L1, RXRA, PLAU, CD63, CD14, RHOC, NPC2, NME6, PDGFRB, MGMT1, ERCC1, RCC1, FUS, TUBA3, CHEK2, APOC1, ABCC10, SRC, TUBB, FLAD1, MAD2L2, LAPTM4B, REG1A, PRKCD, CST7, IGFBP2, FYN, KDR, STMN1, ZWILCH, RBM17, TP53BP1, ABCA9, NTSR2, FOS, TNFRSF10A, MSH3, PTEN, GBP2, STK11, ERBB4, TFF1, ABCC1, CDC25B, TUBD1, BIRC4, ACTR3, SLC35B1, COL1A1, FOXA1, DUSP1, CXCR4, GGPS1, KNS2, RB1, BCL2L1, XIST, BID, BCL2, STAT3, PECAM1, DIABLO, CYBA, TBCE, CYP1B1, APEX1, TBCD, HRAS, TNFRSF10B, ELP3, PIK3C2A, HSPA5, VEGFC, MMP11, SGK, CTSD, BAD, PTPN21, HSPA9B, or PMS1 is negatively correlated with an increased likelihood of a beneficial response to a treatment including a cyclophosphamide, and
wherein the report includes information based on the likelihood of a beneficial response to chemotherapy including a cyclophosphamide.

11. The method of claim 9, wherein the chemotherapy includes an anthracycline.

12. The method of claim 11, wherein the anthracycline is doxorubicin.

13. The method of claim 9, wherein the taxane is docetaxel.

14. A method of predicting whether a hormone receptor (HR) negative cancer patient will exhibit a beneficial response to chemotherapy, comprising
measuring an expression level of a gene, or its expression product, in a tumor sample obtained from the patient, wherein the gene is selected from the group consisting of CD247, TYMS, IGF1R, ACTG2, CCND1, CAPZA1, CHEK2, STMN1, and ZWILCH
using the expression level to determine a likelihood of a beneficial response to a treatment including a taxane,
wherein expression of CD247, TYMS, IGF1R, ACTG2, CAPZA1, CHEK2, STMN1, or ZWILCH is positively correlated with increased likelihood of a beneficial response to a treatment including a taxane, and
wherein expression of CCND1 is negatively correlated with an increased likelihood of a beneficial response to a treatment including a taxane; and
generating a report including information based on the likelihood of a beneficial response to chemotherapy including a taxane.

15. The method of claim 14, wherein the method comprises using the expression level to determine a likelihood of a beneficial response to a treatment including a cyclophosphamide,
wherein expression of CD247, CCND1, or CAPZA1 is positively correlated with increased likelihood of a beneficial response to a treatment including a cyclophosphamide, and
wherein expression of TYMS, IGF1R, ACTG2, CHEK2, STMN1, or ZWILCH is negatively correlated with an increased likelihood of a beneficial response to a treatment including a cyclophosphamide, and
wherein the report includes information based on the likelihood of a beneficial response to chemotherapy including a cyclophosphamide.

16. The method of claim 14, wherein the chemotherapy includes an anthracycline.

17. The method of claim 16, wherein the anthracycline is doxorubicin.

18. The method of claim 14, wherein the taxane is docetaxel.

19. A method of predicting whether a cancer patient will exhibit a beneficial response to chemotherapy, comprising
measuring an expression level of a gene, or its expression product, in a tumor sample obtained from the patient, wherein the gene is selected from the group consisting of ABCC1, ABCC10, ABCC5, ACTB, ACTR2, APEX1, APOC1, APRT, BAK1, BAX, BBC3, BCL2L13, BID, BUB1, BUB3, CAPZA1, CCT3, CD247, CD68, CDCA8, CENPA, CENPF, CHEK2, CHFR, CST7, CXCR4, DDR1, DICER1, EIF4E2, GADD45B, GBP1, HDAC6, HSPA1A, HSPA1B, HSPA1L, 1L2RA, IL7, ILK, KALPHA1, KIF22, LILRB1, LIMK2, MAD2L1, MAPRE1, MCL1, MRE11A, NEK2, NTSR2, PHB, PLD3, RAD1, RALBP1, RHOA, RPN2, SHC1, SLC1A3, SRC, STAT1, STK10, STMN1, TBCC, TOP3B, TPX2, TSPAN4, TUBA3, TUBA6, TUBB, TUBB2C, TUBB3, TYMS, VEGF, VHL, WNT5A, ZW10, ZWILCH, and ZWINT;
using the expression level to determine a likelihood of a beneficial response to a treatment including a taxane,
wherein expression of SLC1A3, TBCC, EIF4E2, TUBB, TSPAN4, VHL, BAX, CD247, CAPZA1, STMN1, ABCC1, ZW10, HSPA1B, MAPRE1, PLD3, APRT, BAK1, CST7, SHC1, ZWILCH, SRC, GADD45B, LIMK2, CHEK2, RAD1, MRE11A, DDR1, STK10, LILRB1, BBC3, BUB3, TOP3B, RPN2, ILK, GBP1, TUBB3, NTSR2, BID, BCL2L13, ABCC5, HDAC6, CD68, DICER1, RHOA, CCT3, ACTR2, WNT5A, HSPA1L, APOC1, APEX1, KALPHA1, ABCC10, PHB, TUBB2C, RALBP1, MCL1, HSPA1A, 1L2RA, TUBA3, ACTB, KIF22, CXCR4, STAT1, IL7, or CHFR is positively correlated with increased likelihood of a beneficial response to a treatment including a taxane, and
wherein expression of CENPA, CDCA8, TPX2, NEK2, TYMS, ZWINT, VEGF, BUB1, MAD2L1, or CENPF is negatively correlated with an increased likelihood of a beneficial response to a treatment including a taxane; and
generating a report including information based on the likelihood of a beneficial response to chemotherapy including a taxane.

20. The method of claim 19, wherein the method comprises using the expression level to determine a likelihood of a beneficial response to a treatment including a cyclophosphamide,
wherein expression of SLC1A3, TSPAN4, BAX, CD247, CAPZA1, ZW10, CST7, SHC1, GADD45B, MRE11A, STK10, LILRB1, BBC3, BUB3, ILK, GBP1, BCL2L13, CD68, DICER1, RHOA, ACTR2, WNT5A, HSPA1L, APEX1, MCL1, IL2RA, ACTB, STAT1, IL7, or CHFR is positively correlated with increased likelihood of a beneficial response to a treatment including a cyclophosphamide, and
wherein expression of TBCC, EIF4E2, TUBB, VHL, STMN1, ABCC1, HSPA1B, MAPRE1, APRT, BAK1, TUBA6, ZWILCH, SRC, LIMK2, CENPA, CHEK2, RAD1, DDR1, CDCA8, TOP3B, RPN2, TUBB3, NTSR2, BID, TPX2, ABCC5, HDAC6, NEK2, TYMS, CCT3, ZWINT, KALPHA1, ABCC10, PHB, TUBB2C, RALBP1, VEGF, HSPA1A, BUB1, MAD2L1, CENPF, TUBA3, KIF22, or CXCR4 is negatively correlated with an increased likelihood of a beneficial response to a treatment including a cyclophosphamide, and
wherein the report includes information based on the likelihood of a beneficial response to chemotherapy including a cyclophosphamide.

21. A kit comprising one or more (1) extraction buffer/reagents and protocol; (2) reverse transcription buffer/reagents and protocol; and (3) qPCR buffer/reagents and protocol, suitable for performing the method of claims 1, 9, 14 or 19.


SEQUENCE LISTING (1496 sequences)

SEQ ID NO: 1 - DNA, 20 bp, Artificial Sequence
  Synthetic Oligonucleotide
ttacccgtgggaactgtctc

SEQ ID NO: 2 - DNA, 22 bp, Artificial Sequence
  Synthetic Oligonucleotide
gaccagtaaatgggtcagagga

SEQ ID NO: 3 - DNA, 25 bp, Artificial Sequence
  Synthetic Oligonucleotide
tcctctcaccaggacaacaaccaca

SEQ ID NO: 4 - DNA, 20 bp, Artificial Sequence
  Synthetic Oligonucleotide
aaacaccactggagcattga

SEQ ID NO: 5 - DNA, 20 bp, Artificial Sequence
  Synthetic Oligonucleotide
caagcctggaacctatagcc

SEQ ID NO: 6 - DNA, 24 bp, Artificial Sequence
  Synthetic Oligonucleotide
ctcgccaatgatgctgctcaagtt

SEQ ID NO: 7 - DNA, 18 bp, Artificial Sequence
  Synthetic Oligonucleotide
agacagtcgccttggtcg

SEQ ID NO: 8 - DNA, 20 bp, Artificial Sequence
  Synthetic Oligonucleotide
aacctctgcagaagctggac

SEQ ID NO: 9 - DNA, 24 bp, Artificial Sequence
  Synthetic Oligonucleotide
ccgtactcttcccactgccattga

SEQ ID NO: 10 - DNA, 18 bp, Artificial Sequence
  Synthetic Oligonucleotide
accagtgccacaatgcag

SEQ ID NO: 11 - DNA, 18 bp, Artificial Sequence
  Synthetic Oligonucleotide
atagcgctgaccactgcc

SEQ ID NO: 12 - DNA, 24 bp, Artificial Sequence
  Synthetic Oligonucleotide
ccatgagctgtagccgaatgtcca

SEQ ID NO: 13 - DNA, 18 bp, Artificial Sequence
  Synthetic Oligonucleotide
aagccacagcctccattg

SEQ ID NO: 14 - DNA, 19 bp, Artificial Sequence
  Synthetic Oligonucleotide
ggaaggcttcacggattgt

SEQ ID NO: 15 - DNA, 24 bp, Artificial Sequence
  Synthetic Oligonucleotide
tggagacagacaccctgatccagc

SEQ ID NO: 16 - DNA, 20 bp, Artificial Sequence
  Synthetic Oligonucleotide
tgcagactgtaccatgctga

SEQ ID NO: 17 - DNA, 20 bp, Artificial Sequence
  Synthetic Oligonucleotide
ggccagcaccataatcctat

SEQ ID NO: 18 - DNA, 22 bp, Artificial Sequence
  Synthetic Oligonucleotide
ctgcacacggttctaggctccg

SEQ ID NO: 19 - DNA, 20 bp, Artificial Sequence
  Synthetic Oligonucleotide
tctgtggcccacctctactc

SEQ ID NO: 20 - DNA, 20 bp, Artificial Sequence
  Synthetic Oligonucleotide
gggtgtaggaagtcacagcc

SEQ ID NO: 21 - DNA, 24 bp, Artificial Sequence
  Synthetic Oligonucleotide
aacctgaccaagccactcctggac

SEQ ID NO: 22 - DNA, 21 bp, Artificial Sequence
  Synthetic Oligonucleotide
atgtacgtcgccattcaagct

SEQ ID NO: 23 - DNA, 19 bp, Artificial Sequence
  Synthetic Oligonucleotide
acgccatcacctgaatcca

SEQ ID NO: 24 - DNA, 20 bp, Artificial Sequence
  Synthetic Oligonucleotide
ctggccgcacgacaggcatc

SEQ ID NO: 25 - DNA, 18 bp, Artificial Sequence
  Synthetic Oligonucleotide
atccgcattgaagaccca

SEQ ID NO: 26 - DNA, 20 bp, Artificial Sequence
  Synthetic Oligonucleotide
atccgctagaactgcaccac

SEQ ID NO: 27 - DNA, 24 bp, Artificial Sequence
  Synthetic Oligonucleotide
cccgcagaaagcacatggtattcc

SEQ ID NO: 28 - DNA, 20 bp, Artificial Sequence
  Synthetic Oligonucleotide
caactgctgagagaccgaga

SEQ ID NO: 29 - DNA, 20 bp, Artificial Sequence
  Synthetic Oligonucleotide
cgctcctttactgccttagc

SEQ ID NO: 30 - DNA, 26 bp, Artificial Sequence
  Synthetic Oligonucleotide
aggaatccctccagaacaatccttgg

SEQ ID NO: 31 - DNA, 27 bp, Artificial Sequence
  Synthetic Oligonucleotide
ctgcatgtgattgaataagaaacaaga

SEQ ID NO: 32 - DNA, 22 bp, Artificial Sequence
  Synthetic Oligonucleotide
tgtggacctgatccctgtacac

SEQ ID NO: 33 - DNA, 23 bp, Artificial Sequence
  Synthetic Oligonucleotide
tgaccacaccaaagcctccctgg

SEQ ID NO: 34 - DNA, 20 bp, Artificial Sequence
  Synthetic Oligonucleotide
cgcttctatggcgctgagat

SEQ ID NO: 35 - DNA, 20 bp, Artificial Sequence
  Synthetic Oligonucleotide
tcccggtacaccacgttctt

SEQ ID NO: 36 - DNA, 24 bp, Artificial Sequence
  Synthetic Oligonucleotide
cagccctggactacctgcactcgg

SEQ ID NO: 37 - DNA, 19 bp, Artificial Sequence
  Synthetic Oligonucleotide
tcctgccacccttcaaacc

SEQ ID NO: 38 - DNA, 21 bp, Artificial Sequence
  Synthetic Oligonucleotide
ggcggtaaattcatcatcgaa

SEQ ID NO: 39 - DNA, 24 bp, Artificial Sequence
  Synthetic Oligonucleotide
caggtcacgtccgaggtcgacaca

SEQ ID NO: 40 - DNA, 25 bp, Artificial Sequence
  Synthetic Oligonucleotide
ttgtctctgccttggactatctaca

SEQ ID NO: 41 - DNA, 24 bp, Artificial Sequence
  Synthetic Oligonucleotide
ccagcattagattctccaacttga

SEQ ID NO: 42 - DNA, 23 bp, Artificial Sequence
  Synthetic Oligonucleotide
tcacggtacacaatctttccgga

SEQ ID NO: 43 - DNA, 19 bp, Artificial Sequence
  Synthetic Oligonucleotide
tgggagggatgaaggaaat

SEQ ID NO: 44 - DNA, 19 bp, Artificial Sequence
  Synthetic Oligonucleotide
ctcatacaggtcctgggca

SEQ ID NO: 45 - DNA, 24 bp, Artificial Sequence
  Synthetic Oligonucleotide
tgtctcacgagagcatcgtccaga

SEQ ID NO: 46 - DNA, 20 bp, Artificial Sequence
  Synthetic Oligonucleotide
ggacagcaggaatgtgtttc

SEQ ID NO: 47 - DNA, 20 bp, Artificial Sequence
  Synthetic Oligonucleotide
acccactcgatttgtttctg

SEQ ID NO: 48 - DNA, 22 bp, Artificial Sequence
  Synthetic Oligonucleotide
cattggctccccgtgacctgta

SEQ ID NO: 49 - DNA, 20 bp, Artificial Sequence
  Synthetic Oligonucleotide
gatgaagcctttcgcaagtt

SEQ ID NO: 50 - DNA, 20 bp, Artificial Sequence
  Synthetic Oligonucleotide
aggtctccacacagcacaag

SEQ ID NO: 51 - DNA, 21 bp, Artificial Sequence
  Synthetic Oligonucleotide
ctttcgggaagccaggccctt

SEQ ID NO: 52 - DNA, 21 bp, Artificial Sequence
  Synthetic Oligonucleotide
ggaaacacactggaggacaag

SEQ ID NO: 53 - DNA, 19 bp, Artificial Sequence
  Synthetic Oligonucleotide
cgcatcttggcagaaagtt

SEQ ID NO: 54 - DNA, 24 bp, Artificial Sequence
  Synthetic Oligonucleotide
tcatcagccgcatcaaacagagtg

SEQ ID NO: 55 - DNA, 18 bp, Artificial Sequence
  Synthetic Oligonucleotide
gtttatgccatcggcacc

SEQ ID NO: 56 - DNA, 22 bp, Artificial Sequence
  Synthetic Oligonucleotide
ggaatacacgagggcatagttc

SEQ ID NO: 57 - DNA, 24 bp, Artificial Sequence
  Synthetic Oligonucleotide
actggatcctggccaccgactatg

SEQ ID NO: 58 - DNA, 18 bp, Artificial Sequence
  Synthetic Oligonucleotide
gcctcaagagctggttcg

SEQ ID NO: 59 - DNA, 18 bp, Artificial Sequence
  Synthetic Oligonucleotide
cctgcaccttctccacca

SEQ ID NO: 60 - DNA, 22 bp, Artificial Sequence
  Synthetic Oligonucleotide
actggcgctgcatgtcttccac

SEQ ID NO: 61 - DNA, 18 bp, Artificial Sequence
  Synthetic Oligonucleotide
gaggtcctggagtgcgtg

SEQ ID NO: 62 - DNA, 18 bp, Artificial Sequence
  Synthetic Oligonucleotide
aggtgccagcttctccct

SEQ ID NO: 63 - DNA, 24 bp, Artificial Sequence
  Synthetic Oligonucleotide
ccttaagcgaggtcagctccacca

SEQ ID NO: 64 - DNA, 18 bp, Artificial Sequence
  Synthetic Oligonucleotide
ggtcctccgtcggttctc

SEQ ID NO: 65 - DNA, 18 bp, Artificial Sequence
  Synthetic Oligonucleotide
gtcgcaaactcggagacg

SEQ ID NO: 66 - DNA, 24 bp, Artificial Sequence
  Synthetic Oligonucleotide
ccacggtctggtcttcagctaccc

SEQ ID NO: 67 - DNA, 21 bp, Artificial Sequence
  Synthetic Oligonucleotide
agctgcagaagagctgcacat

SEQ ID NO: 68 - DNA, 20 bp, Artificial Sequence
  Synthetic Oligonucleotide
gcatctgccaactcctccat

SEQ ID NO: 69 - DNA, 22 bp, Artificial Sequence
  Synthetic Oligonucleotide
tgacgagcagcgaacagccacg

SEQ ID NO: 70 - DNA, 21 bp, Artificial Sequence
  Synthetic Oligonucleotide
cagcagatgtggatcagcaag

SEQ ID NO: 71 - DNA, 18 bp, Artificial Sequence
  Synthetic Oligonucleotide
gcatttgcggtggacgat

SEQ ID NO: 72 - DNA, 23 bp, Artificial Sequence
  Synthetic Oligonucleotide
aggagtatgacgagtccggcccc

SEQ ID NO: 73 - DNA, 22 bp, Artificial Sequence
  Synthetic Oligonucleotide
ggctcttgtgcgtactgtcctt

SEQ ID NO: 74 - DNA, 23 bp, Artificial Sequence
  Synthetic Oligonucleotide
tcagatgacgaagagcacagatg

SEQ ID NO: 75 - DNA, 29 bp, Artificial Sequence
  Synthetic Oligonucleotide
aggctcagtgatgtcttccctgtcaccag

SEQ ID NO: 76 - DNA, 19 bp, Artificial Sequence
  Synthetic Oligonucleotide
gggtcaggtgcctcgagat

SEQ ID NO: 77 - DNA, 21 bp, Artificial Sequence
  Synthetic Oligonucleotide
ctgctcactcggctcaaactc

SEQ ID NO: 78 - DNA, 24 bp, Artificial Sequence
  Synthetic Oligonucleotide
tgggcccagagcatgttccagatc

SEQ ID NO: 79 - DNA, 23 bp, Artificial Sequence
  Synthetic Oligonucleotide
cgttgtcagcacttggaatacaa

SEQ ID NO: 80 - DNA, 24 bp, Artificial Sequence
  Synthetic Oligonucleotide
gttcaacctcttcctgtggactgt

SEQ ID NO: 81 - DNA, 26 bp, Artificial Sequence
  Synthetic Oligonucleotide
cccaattaacatgacccggcaaccat

SEQ ID NO: 82 - DNA, 20 bp, Artificial Sequence
  Synthetic Oligonucleotide
ccattcccaccattctacct

SEQ ID NO: 83 - DNA, 20 bp, Artificial Sequence
  Synthetic Oligonucleotide
gggaacatagacccaccaat

SEQ ID NO: 84 - DNA, 21 bp, Artificial Sequence
  Synthetic Oligonucleotide
acaccccagacgtcctggcct

SEQ ID NO: 85 - DNA, 18 bp, Artificial Sequence
  Synthetic Oligonucleotide
ccgccgtggacacagact

SEQ ID NO: 86 - DNA, 21 bp, Artificial Sequence
  Synthetic Oligonucleotide
ttgccgtcagaaaacatgtca

SEQ ID NO: 87 - DNA, 25 bp, Artificial Sequence
  Synthetic Oligonucleotide
tgccactcggaaaaagacctctcgg

SEQ ID NO: 88 - DNA, 20 bp, Artificial Sequence
  Synthetic Oligonucleotide
cctggagggtcctgtacaat

SEQ ID NO: 89 - DNA, 19 bp, Artificial Sequence
  Synthetic Oligonucleotide
ctaattgggctccatctcg

SEQ ID NO: 90 - DNA, 24 bp, Artificial Sequence
  Synthetic Oligonucleotide
catcatgggactcctgcccttacc

SEQ ID NO: 91 - DNA, 25 bp, Artificial Sequence
  Synthetic Oligonucleotide
cagatggacctagtacccactgaga

SEQ ID NO: 92 - DNA, 24 bp, Artificial Sequence
  Synthetic Oligonucleotide
cctatgatttaagggcatttttcc

SEQ ID NO: 93 - DNA, 22 bp, Artificial Sequence
  Synthetic Oligonucleotide
ttccacgccgaaggacagcgat

SEQ ID NO: 94 - DNA, 20 bp, Artificial Sequence
  Synthetic Oligonucleotide
aattaccaagcagccgaaga

SEQ ID NO: 95 - DNA, 20 bp, Artificial Sequence
  Synthetic Oligonucleotide
caggcggacaatgtaacgta

SEQ ID NO: 96 - DNA, 28 bp, Artificial Sequence
  Synthetic Oligonucleotide
ccacccacgaatggttatcttacgactg

SEQ ID NO: 97 - DNA, 20 bp, Artificial Sequence
  Synthetic Oligonucleotide
cagcgacaactctggacaag

SEQ ID NO: 98 - DNA, 19 bp, Artificial Sequence
  Synthetic Oligonucleotide
gctctcagactgccaggaa

SEQ ID NO: 99 - DNA, 24 bp, Artificial Sequence
  Synthetic Oligonucleotide
ccccagagtctccaactgtgacca

SEQ ID NO: 100 - DNA, 24 bp, Artificial Sequence
  Synthetic Oligonucleotide
cttttgtggaactctatgggaaca

SEQ ID NO: 101 - DNA, 19 bp, Artificial Sequence
  Synthetic Oligonucleotide
cagcggttgaagcgttcct

SEQ ID NO: 102 - DNA, 20 bp, Artificial Sequence
  Synthetic Oligonucleotide
ttcggctctcggctgctgca

SEQ ID NO: 103 - DNA, 26 bp, Artificial Sequence
  Synthetic Oligonucleotide
tgtactggcgaagaatatttggtaaa

SEQ ID NO: 104 - DNA, 20 bp, Artificial Sequence
  Synthetic Oligonucleotide
gccacgtgattcttccacaa

SEQ ID NO: 105 - DNA, 24 bp, Artificial Sequence
  Synthetic Oligonucleotide
cagggcatcgatctctcaccctgg

SEQ ID NO: 106 - DNA, 20 bp, Artificial Sequence
  Synthetic Oligonucleotide
ggactgtgaggtcaacaacg

SEQ ID NO: 107 - DNA, 20 bp, Artificial Sequence
  Synthetic Oligonucleotide
ggaagccaaacaccagtagg

SEQ ID NO: 108 - DNA, 24 bp, Artificial Sequence
  Synthetic Oligonucleotide
tgtgatgcactcatccctgaggct

SEQ ID NO: 109 - DNA, 20 bp, Artificial Sequence
  Synthetic Oligonucleotide
cctgcaaaagggaacaagag

SEQ ID NO: 110 - DNA, 20 bp, Artificial Sequence
  Synthetic Oligonucleotide
cgtggttgactctgatctcg

SEQ ID NO: 111 - DNA, 21 bp, Artificial Sequence
  Synthetic Oligonucleotide
cttcgcctccagatggctccc

SEQ ID NO: 112 - DNA, 20 bp, Artificial Sequence
  Synthetic Oligonucleotide
tcagggggctagaaatctgt

SEQ ID NO: 113 - DNA, 20 bp, Artificial Sequence
  Synthetic Oligonucleotide
ccattccagttgatctgtgg

SEQ ID NO: 114 - DNA, 23 bp, Artificial Sequence
  Synthetic Oligonucleotide
ctatgggcccttcaccaacatgc

SEQ ID NO: 115 - DNA, 20 bp, Artificial Sequence
  Synthetic Oligonucleotide
agttcgtgctttgcaagatg

SEQ ID NO: 116 - DNA, 20 bp, Artificial Sequence
  Synthetic Oligonucleotide
aaggtaagctgggtctgctg

SEQ ID NO: 117 - DNA, 29 bp, Artificial Sequence
  Synthetic Oligonucleotide
cattcttcactgcttcataaagctctgca

SEQ ID NO: 118 - DNA, 21 bp, Artificial Sequence
  Synthetic Oligonucleotide
ccgaggttaatccagcacgta

SEQ ID NO: 119 - DNA, 21 bp, Artificial Sequence
  Synthetic Oligonucleotide
aagacatggcgctctcagttc

SEQ ID NO: 120 - DNA, 23 bp, Artificial Sequence
  Synthetic Oligonucleotide
tgctgggagcctacacttggccc

SEQ ID NO: 121 - DNA, 24 bp, Artificial Sequence
  Synthetic Oligonucleotide
tcaacagaaggctgaaccactaga

SEQ ID NO: 122 - DNA, 22 bp, Artificial Sequence
  Synthetic Oligonucleotide
caacagagtttgccgagacact

SEQ ID NO: 123 - DNA, 24 bp, Artificial Sequence
  Synthetic Oligonucleotide
tacagtcccagcaccgacaattcc

SEQ ID NO: 124 - DNA, 22 bp, Artificial Sequence
  Synthetic Oligonucleotide
ctgaagcagatggttcatcatt

SEQ ID NO: 125 - DNA, 22 bp, Artificial Sequence
  Synthetic Oligonucleotide
gctgattcccaagagtctaacc

SEQ ID NO: 126 - DNA, 24 bp, Artificial Sequence
  Synthetic Oligonucleotide
cctcgctttgtttaacagcccagg

SEQ ID NO: 127 - DNA, 23 bp, Artificial Sequence
  Synthetic Oligonucleotide
tgaggagtggtattttggcaaga

SEQ ID NO: 128 - DNA, 21 bp, Artificial Sequence
  Synthetic Oligonucleotide
ctctcgggttctctgcattga

SEQ ID NO: 129 - DNA, 24 bp, Artificial Sequence
  Synthetic Oligonucleotide
aaccgctctgactcccgtctggtg

SEQ ID NO: 130 - DNA, 20 bp, Artificial Sequence
  Synthetic Oligonucleotide
gtcagcgtggtagcggtatt

SEQ ID NO: 131 - DNA, 21 bp, Artificial Sequence
  Synthetic Oligonucleotide
ggaagtcttggctaaagaggc

SEQ ID NO: 132 - DNA, 24 bp, Artificial Sequence
  Synthetic Oligonucleotide
aacaattactgtcactgccgcgga

SEQ ID NO: 133 - DNA, 20 bp, Artificial Sequence
  Synthetic Oligonucleotide
tcagctgtgagctgcggata

SEQ ID NO: 134 - DNA, 24 bp, Artificial Sequence
  Synthetic Oligonucleotide
acggtcctaggtttgaggttaaga

SEQ ID NO: 135 - DNA, 19 bp, Artificial Sequence
  Synthetic Oligonucleotide
caggtcccattgccgggcg

SEQ ID NO: 136 - DNA, 20 bp, Artificial Sequence
  Synthetic Oligonucleotide
atcctagccctggtttttgg

SEQ ID NO: 137 - DNA, 20 bp, Artificial Sequence
  Synthetic Oligonucleotide
ctgccttctcatctgcacaa

SEQ ID NO: 138 - DNA, 20 bp, Artificial Sequence
  Synthetic Oligonucleotide
tttgctgtcaccagcgtcgc

SEQ ID NO: 139 - DNA, 26 bp, Artificial Sequence
  Synthetic Oligonucleotide
cactaaggtttgagacagttccagaa

SEQ ID NO: 140 - DNA, 23 bp, Artificial Sequence
  Synthetic Oligonucleotide
gcgaattagccctctacaactga

SEQ ID NO: 141 - DNA, 26 bp, Artificial Sequence
  Synthetic Oligonucleotide
aacccaagctcaagacgcaggacgag

SEQ ID NO: 142 - DNA, 20 bp, Artificial Sequence
  Synthetic Oligonucleotide
tcgttggagatcagagtgga

SEQ ID NO: 143 - DNA, 18 bp, Artificial Sequence
  Synthetic Oligonucleotide
ttaagcacgccaaccacc

SEQ ID NO: 144 - DNA, 24 bp, Artificial Sequence
  Synthetic Oligonucleotide
tcaccatcacaccacctacagccc

SEQ ID NO: 145 - DNA, 20 bp, Artificial Sequence
  Synthetic Oligonucleotide
gtggctcaacattgtgttcc

SEQ ID NO: 146 - DNA, 20 bp, Artificial Sequence
  Synthetic Oligonucleotide
caatggcctccattttacag

SEQ ID NO: 147 - DNA, 24 bp, Artificial Sequence
  Synthetic Oligonucleotide
atttcagctgatcagtgggcctcc

SEQ ID NO: 148 - DNA, 20 bp, Artificial Sequence
  Synthetic Oligonucleotide
ttcaggttgttgcaggagac

SEQ ID NO: 149 - DNA, 20 bp, Artificial Sequence
  Synthetic Oligonucleotide
catcttcttgggcacacaat

SEQ ID NO: 150 - DNA, 27 bp, Artificial Sequence
  Synthetic Oligonucleotide
tgtctccattattgatcggttcatgca

SEQ ID NO: 151 - DNA, 21 bp, Artificial Sequence
  Synthetic Oligonucleotide
gcatgttcgtggcctctaaga

SEQ ID NO: 152 - DNA, 22 bp, Artificial Sequence
  Synthetic Oligonucleotide
cggtgtagatgcacagcttctc

SEQ ID NO: 153 - DNA, 23 bp, Artificial Sequence
  Synthetic Oligonucleotide
aaggagaccatccccctgacggc

SEQ ID NO: 154 - DNA, 22 bp, Artificial Sequence
  Synthetic Oligonucleotide
atgctgtggctccttcctaact

SEQ ID NO: 155 - DNA, 27 bp, Artificial Sequence
  Synthetic Oligonucleotide
acccaaattgtgatatacaaaaaggtt

SEQ ID NO: 156 - DNA, 30 bp, Artificial Sequence
  Synthetic Oligonucleotide
taccaagcaacctacatgtcaagaaagccc

SEQ ID NO: 157 - DNA, 18 bp, Artificial Sequence
  Synthetic Oligonucleotide
atccaaggccatgactgg

SEQ ID NO: 158 - DNA, 20 bp, Artificial Sequence
  Synthetic Oligonucleotide
ggaatgacctctagggcctg

SEQ ID NO: 159 - DNA, 24 bp, Artificial Sequence
  Synthetic Oligonucleotide
acagccctgtatggccattgttcc

SEQ ID NO: 160 - DNA, 19 bp, Artificial Sequence
  Synthetic Oligonucleotide
gtgtgctagcgtactcccg

SEQ ID NO: 161 - DNA, 18 bp, Artificial Sequence
  Synthetic Oligonucleotide
gcatggtgccggttatct

SEQ ID NO: 162 - DNA, 24 bp, Artificial Sequence
  Synthetic Oligonucleotide
caaggaactgacgctcgaggacct

SEQ ID NO: 163 - DNA, 25 bp, Artificial Sequence
  Synthetic Oligonucleotide
tgtatttcaagacctctgtgcactt

SEQ ID NO: 164 - DNA, 23 bp, Artificial Sequence
  Synthetic Oligonucleotide
ttagcctgaggaattgctgtgtt

SEQ ID NO: 165 - DNA, 25 bp, Artificial Sequence
  Synthetic Oligonucleotide
tttatgaacctgccctgctcccaca

SEQ ID NO: 166 - DNA, 20 bp, Artificial Sequence
  Synthetic Oligonucleotide
agatgaagtggaaggcgctt

SEQ ID NO: 167 - DNA, 21 bp, Artificial Sequence
  Synthetic Oligonucleotide
tgcctctgtaatcggcaactg

SEQ ID NO: 168 - DNA, 18 bp, Artificial Sequence
  Synthetic Oligonucleotide
caccgcggccatcctgca

SEQ ID NO: 169 - DNA, 18 bp, Artificial Sequence
  Synthetic Oligonucleotide
agtgggactgattgccgt

SEQ ID NO: 170 - DNA, 19 bp, Artificial Sequence
  Synthetic Oligonucleotide
gggtagccccctggattat

SEQ ID NO: 171 - DNA, 24 bp, Artificial Sequence
  Synthetic Oligonucleotide
tctgactcaggacaagctgtgccc

SEQ ID NO: 172 - DNA, 18 bp, Artificial Sequence
  Synthetic Oligonucleotide
tggttcccagccctgtgt

SEQ ID NO: 173 - DNA, 19 bp, Artificial Sequence
  Synthetic Oligonucleotide
ctcctccaccctgggttgt

SEQ ID NO: 174 - DNA, 28 bp, Artificial Sequence
  Synthetic Oligonucleotide
ctccaagcccagattcagattcgagtca

SEQ ID NO: 175 - DNA, 18 bp, Artificial Sequence
  Synthetic Oligonucleotide
gagagcgacgcggttgtt

SEQ ID NO: 176 - DNA, 26 bp, Artificial Sequence
  Synthetic Oligonucleotide
gtatggtagatcccggcttattattc

SEQ ID NO: 177 - DNA, 18 bp, Artificial Sequence
  Synthetic Oligonucleotide
tagctgccgctgcggccg

SEQ ID NO: 178 - DNA, 21 bp, Artificial Sequence
  Synthetic Oligonucleotide
tggattggagttctgggaatg

SEQ ID NO: 179 - DNA, 22 bp, Artificial Sequence
  Synthetic Oligonucleotide
gcttgcactccacaggtacaca

SEQ ID NO: 180 - DNA, 23 bp, Artificial Sequence
  Synthetic Oligonucleotide
actggccgtggcactggacaaca

SEQ ID NO: 181 - DNA, 21 bp, Artificial Sequence
  Synthetic Oligonucleotide
aaacgagcagtttgccatcag

SEQ ID NO: 182 - DNA, 20 bp, Artificial Sequence
  Synthetic Oligonucleotide
gttggtgatgttccgaagca

SEQ ID NO: 183 - DNA, 24 bp, Artificial Sequence
  Synthetic Oligonucleotide
cctcaccggcatagactggaagcg

SEQ ID NO: 184 - DNA, 19 bp, Artificial Sequence
  Synthetic Oligonucleotide
gaggcacagtattgcccag

SEQ ID NO: 185 - DNA, 21 bp, Artificial Sequence
  Synthetic Oligonucleotide
gagacggttggagagcttctt

SEQ ID NO: 186 - DNA, 24 bp, Artificial Sequence
  Synthetic Oligonucleotide
atgtttcccaaggcctctggatcc

SEQ ID NO: 187 - DNA, 21 bp, Artificial Sequence
  Synthetic Oligonucleotide
tgagtgtcccccggtatcttc

SEQ ID NO: 188 - DNA, 21 bp, Artificial Sequence
  Synthetic Oligonucleotide
cagccgctttcagattttcat

SEQ ID NO: 189 - DNA, 27 bp, Artificial Sequence
  Synthetic Oligonucleotide
tgccaatcccgatgaaattggaaattt

SEQ ID NO: 190 - DNA, 20 bp, Artificial Sequence
  Synthetic Oligonucleotide
aagccctatccgatgtaccc

SEQ ID NO: 191 - DNA, 19 bp, Artificial Sequence
  Synthetic Oligonucleotide
ctgtggcattgagtttggg

SEQ ID NO: 192 - DNA, 24 bp, Artificial Sequence
  Synthetic Oligonucleotide
cacaacatccctggtgaacgtcgt

SEQ ID NO: 193 - DNA, 18 bp, Artificial Sequence
  Synthetic Oligonucleotide
cggcgatcaagaagctgt

SEQ ID NO: 194 - DNA, 18 bp, Artificial Sequence
  Synthetic Oligonucleotide
caggcgctgatctcttgc

SEQ ID NO: 195 - DNA, 24 bp, Artificial Sequence
  Synthetic Oligonucleotide
cgggcctctgatctccgatttctt

SEQ ID NO: 196 - DNA, 21 bp, Artificial Sequence
  Synthetic Oligonucleotide
tgacaatcagcacacctgcat

SEQ ID NO: 197 - DNA, 23 bp, Artificial Sequence
  Synthetic Oligonucleotide
tgtgactacagccgtgatcctta

SEQ ID NO: 198 - DNA, 20 bp, Artificial Sequence
  Synthetic Oligonucleotide
caggccctcttccgagcggt

SEQ ID NO: 199 - DNA, 21 bp, Artificial Sequence
  Synthetic Oligonucleotide
taaattcactcgtggtgtgga

SEQ ID NO: 200 - DNA, 20 bp, Artificial Sequence
  Synthetic Oligonucleotide
gcctcttgtagggccaatag

SEQ ID NO: 201 - DNA, 21 bp, Artificial Sequence
  Synthetic Oligonucleotide
cttcaattggcaagcccaggc

SEQ ID NO: 202 - DNA, 20 bp, Artificial Sequence
  Synthetic Oligonucleotide
ggatgctggtgacctcttct

SEQ ID NO: 203 - DNA, 20 bp, Artificial Sequence
  Synthetic Oligonucleotide
gccaaggcaccaagtaactc

SEQ ID NO: 204 - DNA, 25 bp, Artificial Sequence
  Synthetic Oligonucleotide
tccctcacgttgcaacaggaattaa

SEQ ID NO: 205 - DNA, 18 bp, Artificial Sequence
  Synthetic Oligonucleotide
ctcccgtcaacagcgttc

SEQ ID NO: 206 - DNA, 18 bp, Artificial Sequence
  Synthetic Oligonucleotide
gggtgagtctggccttca

SEQ ID NO: 207 - DNA, 24 bp, Artificial Sequence
  Synthetic Oligonucleotide
acactggaccaggagtgcatccag

SEQ ID NO: 208 - DNA, 20 bp, Artificial Sequence
  Synthetic Oligonucleotide
ctgaaggagctccaagacct

SEQ ID NO: 209 - DNA, 20 bp, Artificial Sequence
  Synthetic Oligonucleotide
caaaaccgctgtgtttcttc

SEQ ID NO: 210 - DNA, 22 bp, Artificial Sequence
  Synthetic Oligonucleotide
tgctgatgtgccctctccttgg

SEQ ID NO: 211 - DNA, 20 bp, Artificial Sequence
  Synthetic Oligonucleotide
aaggaagtggtccctctgtg

SEQ ID NO: 212 - DNA, 20 bp, Artificial Sequence
  Synthetic Oligonucleotide
gacgcagtctttctgtctgg

SEQ ID NO: 213 - DNA, 24 bp, Artificial Sequence
  Synthetic Oligonucleotide
tgaagtctccagctttgcctcagc

SEQ ID NO: 214 - DNA, 26 bp, Artificial Sequence
  Synthetic Oligonucleotide
gataaattggtacaagggatcagctt

SEQ ID NO: 215 - DNA, 24 bp, Artificial Sequence
  Synthetic Oligonucleotide
gggtgccaagtaactgactattca

SEQ ID NO: 216 - DNA, 26 bp, Artificial Sequence
  Synthetic Oligonucleotide
ccagcccacatgtcctgatcatatgc

SEQ ID NO: 217 - DNA, 21 bp, Artificial Sequence
  Synthetic Oligonucleotide
atgtggaacccccacctactt

SEQ ID NO: 218 - DNA, 22 bp, Artificial Sequence
  Synthetic Oligonucleotide
cagtccacagcacggttatacc

SEQ ID NO: 219 - DNA, 29 bp, Artificial Sequence
  Synthetic Oligonucleotide
agtcccaacagaaacaagaacttcaggcg

SEQ ID NO: 220 - DNA, 24 bp, Artificial Sequence
  Synthetic Oligonucleotide
ggatatttccgtggctcttattca

SEQ ID NO: 221 - DNA, 25 bp, Artificial Sequence
  Synthetic Oligonucleotide
cttctcatcaaggcagaaaaatctt

SEQ ID NO: 222 - DNA, 30 bp, Artificial Sequence
  Synthetic Oligonucleotide
tctccatcaaatcctgtaaactccagagca

SEQ ID NO: 223 - DNA, 18 bp, Artificial Sequence
  Synthetic Oligonucleotide
tcattgaccacagtggcg

SEQ ID NO: 224 - DNA, 19 bp, Artificial Sequence
  Synthetic Oligonucleotide
tcgtgtacttctccacccg

SEQ ID NO: 225 - DNA, 24 bp, Artificial Sequence
  Synthetic Oligonucleotide
cacgtcctcatactcaccaaggcg

SEQ ID NO: 226 - DNA, 23 bp, Artificial Sequence
  Synthetic Oligonucleotide
ccccaggatacctaccactacct

SEQ ID NO: 227 - DNA, 18 bp, Artificial Sequence
  Synthetic Oligonucleotide
tgcgggacttgggaaaga

SEQ ID NO: 228 - DNA, 20 bp, Artificial Sequence
  Synthetic Oligonucleotide
cccttcagcctgccccaccg

SEQ ID NO: 229 - DNA, 22 bp, Artificial Sequence
  Synthetic Oligonucleotide
gacatttccagtcctgcagtca

SEQ ID NO: 230 - DNA, 20 bp, Artificial Sequence
  Synthetic Oligonucleotide
ctccgatcgcacacatttgt

SEQ ID NO: 231 - DNA, 23 bp, Artificial Sequence
  Synthetic Oligonucleotide
tgcctctctgccccaccctttgt

SEQ ID NO: 232 - DNA, 21 bp, Artificial Sequence
  Synthetic Oligonucleotide
tccctccactcggaaggacta

SEQ ID NO: 233 - DNA, 22 bp, Artificial Sequence
  Synthetic Oligonucleotide
cggttgttgctgatctgtctca

SEQ ID NO: 234 - DNA, 27 bp, Artificial Sequence
  Synthetic Oligonucleotide
tctgacactgtccaacttgaccctctt

SEQ ID NO: 235 - DNA, 18 bp, Artificial Sequence
  Synthetic Oligonucleotide
tccaccctcctggctttg

SEQ ID NO: 236 - DNA, 21 bp, Artificial Sequence
  Synthetic Oligonucleotide
tcactcccacgttcaccttgt

SEQ ID NO: 237 - DNA, 23 bp, Artificial Sequence
  Synthetic Oligonucleotide
tcctttcgtcttcgccatgctgg

SEQ ID NO: 238 - DNA, 18 bp, Artificial Sequence
  Synthetic Oligonucleotide
gtggccatccagctgacc

SEQ ID NO: 239 - DNA, 23 bp, Artificial Sequence
  Synthetic Oligonucleotide
cagtggtaggtgatgttctggga

SEQ ID NO: 240 - DNA, 21 bp, Artificial Sequence
  Synthetic Oligonucleotide
tcctgcgcctgatgtccaccg

SEQ ID NO: 241 - DNA, 24 bp, Artificial Sequence
  Synthetic Oligonucleotide
cagccaagaactggtataggagct

SEQ ID NO: 242 - DNA, 19 bp, Artificial Sequence
  Synthetic Oligonucleotide
aaactggctgccagcattg

SEQ ID NO: 243 - DNA, 30 bp, Artificial Sequence
  Synthetic Oligonucleotide
tctcctagccagacgtgtttcttgtccttg

SEQ ID NO: 244 - DNA, 21 bp, Artificial Sequence
  Synthetic Oligonucleotide
gagagcaagcgagacattctg

SEQ ID NO: 245 - DNA, 18 bp, Artificial Sequence
  Synthetic Oligonucleotide
aacagggaactggcccac

SEQ ID NO: 246 - DNA, 24 bp, Artificial Sequence
  Synthetic Oligonucleotide
cctctttgacggctcagccaatct

SEQ ID NO: 247 - DNA, 24 bp, Artificial Sequence
  Synthetic Oligonucleotide
cgacagttgcgatgaaagttctaa

SEQ ID NO: 248 - DNA, 22 bp, Artificial Sequence
  Synthetic Oligonucleotide
ggctgctagagaccatggacat

SEQ ID NO: 249 - DNA, 27 bp, Artificial Sequence
  Synthetic Oligonucleotide
cctcctcctgttgctgccactaatgct

SEQ ID NO: 250 - DNA, 23 bp, Artificial Sequence
  Synthetic Oligonucleotide
tctgcagagttggaagcactcta

SEQ ID NO: 251 - DNA, 21 bp, Artificial Sequence
  Synthetic Oligonucleotide
gccgaggcttttctaccagaa

SEQ ID NO: 252 - DNA, 28 bp, Artificial Sequence
  Synthetic Oligonucleotide
caggatacagctccacagcatcgatgtc

SEQ ID NO: 253 - DNA, 20 bp, Artificial Sequence
  Synthetic Oligonucleotide
acctctgtggtccgtaggag

SEQ ID NO: 254 - DNA, 20 bp, Artificial Sequence
  Synthetic Oligonucleotide
cgaccacttgttttccactg

SEQ ID NO: 255 - DNA, 24 bp, Artificial Sequence
  Synthetic Oligonucleotide
tcttcccagggccctcctcatagt

SEQ ID NO: 256 - DNA, 20 bp, Artificial Sequence
  Synthetic Oligonucleotide
aacttcaaggtcggagaagg

SEQ ID NO: 257 - DNA, 20 bp, Artificial Sequence
  Synthetic Oligonucleotide
tggctaaactcctgcacttg

SEQ ID NO: 258 - DNA, 21 bp, Artificial Sequence
  Synthetic Oligonucleotide
ccgtccacggtctcctcctca

SEQ ID NO: 259 - DNA, 18 bp, Artificial Sequence
  Synthetic Oligonucleotide
gtgctacgccaccctgtt

SEQ ID NO: 260 - DNA, 20 bp, Artificial Sequence
  Synthetic Oligonucleotide
caggggcttctcgtagatgt

SEQ ID NO: 261 - DNA, 22 bp, Artificial Sequence
  Synthetic Oligonucleotide
ccgatgttcacgcctttgggtc

SEQ ID NO: 262 - DNA, 20 bp, Artificial Sequence
  Synthetic Oligonucleotide
gatgtgattgaggtgcatgg

SEQ ID NO: 263 - DNA, 21 bp, Artificial Sequence
  Synthetic Oligonucleotide
gaactccctggagatgaaacc

SEQ ID NO: 264 - DNA, 24 bp, Artificial Sequence
  Synthetic Oligonucleotide
tgttcatcctggcgctcttcatgt

SEQ ID NO: 265 - DNA, 18 bp, Artificial Sequence
  Synthetic Oligonucleotide
tgcagcggctgattgaca

SEQ ID NO: 266 - DNA, 25 bp, Artificial Sequence
  Synthetic Oligonucleotide
caactgttcctggtctacaaactca

SEQ ID NO: 267 - DNA, 28 bp, Artificial Sequence
  Synthetic Oligonucleotide
tcagatggagacctcgtgccaaattaca

SEQ ID NO: 268 - DNA, 20 bp, Artificial Sequence
  Synthetic Oligonucleotide
agcttttccggaatctgttc

SEQ ID NO: 269 - DNA, 20 bp, Artificial Sequence
  Synthetic Oligonucleotide
atttgagcatgttccagtcg

SEQ ID NO: 270 - DNA, 24 bp, Artificial Sequence
  Synthetic Oligonucleotide
catcgccagggcttctcctatgac

SEQ ID NO: 271 - DNA, 20 bp, Artificial Sequence
  Synthetic Oligonucleotide
tggcagaactacctgcaaga

SEQ ID NO: 272 - DNA, 19 bp, Artificial Sequence
  Synthetic Oligonucleotide
tgcttcaaggtgtggttgg

SEQ ID NO: 273 - DNA, 24 bp, Artificial Sequence
  Synthetic Oligonucleotide
cacctgcgtctggatgactgtgac

SEQ ID NO: 274 - DNA, 24 bp, Artificial Sequence
  Synthetic Oligonucleotide
gtacatgatcccctgtgagaaggt

SEQ ID NO: 275 - DNA, 21 bp, Artificial Sequence
  Synthetic Oligonucleotide
gggacagcttgtagcctttgc

SEQ ID NO: 276 - DNA, 22 bp, Artificial Sequence
  Synthetic Oligonucleotide
accctgcccgcgatcacactga

SEQ ID NO: 277 - DNA, 23 bp, Artificial Sequence
  Synthetic Oligonucleotide
gggaggcttatctcactgagtga

SEQ ID NO: 278 - DNA, 19 bp, Artificial Sequence
  Synthetic Oligonucleotide
ccattgcagccttcattgc

SEQ ID NO: 279 - DNA, 29 bp, Artificial Sequence
  Synthetic Oligonucleotide
ttgaggcccagagcagtctaccagattct

SEQ ID NO: 280 - DNA, 21 bp, Artificial Sequence
  Synthetic Oligonucleotide
tgtctcactgagcgagcagaa

SEQ ID NO: 281 - DNA, 19 bp, Artificial Sequence
  Synthetic Oligonucleotide
accattgcagccctgattg

SEQ ID NO: 282 - DNA, 24 bp, Artificial Sequence
  Synthetic Oligonucleotide
cttgaggacgcgaacagtccacca

SEQ ID NO: 283 - DNA, 20 bp, Artificial Sequence
  Synthetic Oligonucleotide
tgaccgcttctaccccaatg

SEQ ID NO: 284 - DNA, 22 bp, Artificial Sequence
  Synthetic Oligonucleotide
aggataaggccaaccatgatgt

SEQ ID NO: 285 - DNA, 28 bp, Artificial Sequence
  Synthetic Oligonucleotide
ctgaaactggaacacaaccacccacaag

SEQ ID NO: 286 - DNA, 19 bp, Artificial Sequence
  Synthetic Oligonucleotide
ggtgcctactccattgtgg

SEQ ID NO: 287 - DNA, 20 bp, Artificial Sequence
  Synthetic Oligonucleotide
gtggagcccttcttcctctt

SEQ ID NO: 288 - DNA, 24 bp, Artificial Sequence
  Synthetic Oligonucleotide
tactccagcaggcacacaaacacg

SEQ ID NO: 289 - DNA, 22 bp, Artificial Sequence
  Synthetic Oligonucleotide
ccagctttgtgcctgtcactat

SEQ ID NO: 290 - DNA, 20 bp, Artificial Sequence
  Synthetic Oligonucleotide
gggaatgtggtagcccaaga

SEQ ID NO: 291 - DNA, 25 bp, Artificial Sequence
  Synthetic Oligonucleotide
ctcatgccaccactgccaacacctc

SEQ ID NO: 292 - DNA, 20 bp, Artificial Sequence
  Synthetic Oligonucleotide
ccgtgttcaagaggaagctc

SEQ ID NO: 293 - DNA, 20 bp, Artificial Sequence
  Synthetic Oligonucleotide
agtgggatcacagggtgaag

SEQ ID NO: 294 - DNA, 24 bp, Artificial Sequence
  Synthetic Oligonucleotide
ttttctcaactcctccacaaggca

SEQ ID NO: 295 - DNA, 30 bp, Artificial Sequence
  Synthetic Oligonucleotide
agaacaaggacaacatagatccttacatat

SEQ ID NO: 296 - DNA, 19 bp, Artificial Sequence
  Synthetic Oligonucleotide
gcaaacctcatgccaatgc

SEQ ID NO: 297 - DNA, 26 bp, Artificial Sequence
  Synthetic Oligonucleotide
cacaccctttggaagtggacccagaa

SEQ ID NO: 298 - DNA, 18 bp, Artificial Sequence
  Synthetic Oligonucleotide
ccgtgtggctcgctttct

SEQ ID NO: 299 - DNA, 24 bp, Artificial Sequence
  Synthetic Oligonucleotide
ggagatttcgctgaagagtaacca

SEQ ID NO: 300 - DNA, 20 bp, Artificial Sequence
  Synthetic Oligonucleotide
tgccgcttcctctttgcggg

SEQ ID NO: 301 - DNA, 19 bp, Artificial Sequence
  Synthetic Oligonucleotide
cacaatggcggctctgaag

SEQ ID NO: 302 - DNA, 26 bp, Artificial Sequence
  Synthetic Oligonucleotide
acacaaacactgtctgtacctgaaga

SEQ ID NO: 303 - DNA, 23 bp, Artificial Sequence
  Synthetic Oligonucleotide
aagttacgctgcgcgacagccaa

SEQ ID NO: 304 - DNA, 20 bp, Artificial Sequence
  Synthetic Oligonucleotide
caagcagtcaaggagaacca

SEQ ID NO: 305 - DNA, 20 bp, Artificial Sequence
  Synthetic Oligonucleotide
agttttgctcgcctcatctt

SEQ ID NO: 306 - DNA, 22 bp, Artificial Sequence
  Synthetic Oligonucleotide
ttcttctgtctcccgccgcttc

SEQ ID NO: 307 - DNA, 20 bp, Artificial Sequence
  Synthetic Oligonucleotide
tccaattccagcatcactgt

SEQ ID NO: 308 - DNA, 20 bp, Artificial Sequence
  Synthetic Oligonucleotide
ggcagtgaaggcgataaagt

SEQ ID NO: 309 - DNA, 25 bp, Artificial Sequence
  Synthetic Oligonucleotide
agaaaagctgtttgtctccccagca

SEQ ID NO: 310 - DNA, 20 bp, Artificial Sequence
  Synthetic Oligonucleotide
cagtgcttccatggacaagt

SEQ ID NO: 311 - DNA, 20 bp, Artificial Sequence
  Synthetic Oligonucleotide
tggacagggatgattgatgt

SEQ ID NO: 312 - DNA, 24 bp, Artificial Sequence
  Synthetic Oligonucleotide
atctccatcagcatgggccagttt

SEQ ID NO: 313 - DNA, 21 bp, Artificial Sequence
  Synthetic Oligonucleotide
tgcacagagggtgtgggttac

SEQ ID NO: 314 - DNA, 28 bp, Artificial Sequence
  Synthetic Oligonucleotide
tcttcatctgatttacaagctgtacatg

SEQ ID NO: 315 - DNA, 29 bp, Artificial Sequence
  Synthetic Oligonucleotide
caatgcttccaacaatttgtttgcttgcc

SEQ ID NO: 316 - DNA, 24 bp, Artificial Sequence
  Synthetic Oligonucleotide
ctctgagacagtgcttcgatgact

SEQ ID NO: 317 - DNA, 19 bp, Artificial Sequence
  Synthetic Oligonucleotide
ccatgaggcccaacttcct

SEQ ID NO: 318 - DNA, 23 bp, Artificial Sequence
  Synthetic Oligonucleotide
cagacttggtgccctttgactcc

SEQ ID NO: 319 - DNA, 20 bp, Artificial Sequence
  Synthetic Oligonucleotide
agacatcagctcctggttca

SEQ ID NO: 320 - DNA, 20 bp, Artificial Sequence
  Synthetic Oligonucleotide
gacaaacacccttcctccag

SEQ ID NO: 321 - DNA, 26 bp, Artificial Sequence
  Synthetic Oligonucleotide
cgaggccattgacttcatagactcca

SEQ ID NO: 322 - DNA, 22 bp, Artificial Sequence
  Synthetic Oligonucleotide
cagaggatgacgaggatgatga

SEQ ID NO: 323 - DNA, 18 bp, Artificial Sequence
  Synthetic Oligonucleotide
ctgtgccgcctccttgtc

SEQ ID NO: 324 - DNA, 28 bp, Artificial Sequence
  Synthetic Oligonucleotide
ctcctcattgtcactgccaaacaggtca

SEQ ID NO: 325 - DNA, 20 bp, Artificial Sequence
  Synthetic Oligonucleotide
tgtcgatggacttccagaac

SEQ ID NO: 326 - DNA, 19 bp, Artificial Sequence
  Synthetic Oligonucleotide
attgggacagcttggatca

SEQ ID NO: 327 - DNA, 18 bp, Artificial Sequence
  Synthetic Oligonucleotide
cacctgggcagctgccaa

SEQ ID NO: 328 - DNA, 23 bp, Artificial Sequence
  Synthetic Oligonucleotide
gatctaagatggcgactgtcgaa

SEQ ID NO: 329 - DNA, 25 bp, Artificial Sequence
  Synthetic Oligonucleotide
ttagattccgttttctcctcttctg

SEQ ID NO: 330 - DNA, 27 bp, Artificial Sequence
  Synthetic Oligonucleotide
accacccctactcctaatcccccgact

SEQ ID NO: 331 - DNA, 18 bp, Artificial Sequence
  Synthetic Oligonucleotide
aagccgcggttgaatgtg

SEQ ID NO: 332 - DNA, 20 bp, Artificial Sequence
  Synthetic Oligonucleotide
tgacgccagcttcaatgatg

SEQ ID NO: 333 - DNA, 25 bp, Artificial Sequence
  Synthetic Oligonucleotide
tgaccctctccctctctggatggca

SEQ ID NO: 334 - DNA, 18 bp, Artificial Sequence
  Synthetic Oligonucleotide
ctcggatcctagccctcg

SEQ ID NO: 335 - DNA, 22 bp, Artificial Sequence
  Synthetic Oligonucleotide
ggcattggaatatccctctgta

SEQ ID NO: 336 - DNA, 24 bp, Artificial Sequence
  Synthetic Oligonucleotide
cctccatggactcgagtgtaccga

SEQ ID NO: 337 - DNA, 20 bp, Artificial Sequence
  Synthetic Oligonucleotide
tggtccatcgccagttatca

SEQ ID NO: 338 - DNA, 23 bp, Artificial Sequence
  Synthetic Oligonucleotide
tgttctagcgatcttgcttcaca

SEQ ID NO: 339 - DNA, 30 bp, Artificial Sequence
  Synthetic Oligonucleotide
atctgtatgcggaacctcaaaagagtccct

SEQ ID NO: 340 - DNA, 23 bp, Artificial Sequence
  Synthetic Oligonucleotide
cggttatgtcatgccagatacac

SEQ ID NO: 341 - DNA, 24 bp, Artificial Sequence
  Synthetic Oligonucleotide
gaactgagacccactgaagaaagg

SEQ ID NO: 342 - DNA, 25 bp, Artificial Sequence
  Synthetic Oligonucleotide
cctcaaaggtactccctcctcccgg

SEQ ID NO: 343 - DNA, 25 bp, Artificial Sequence
  Synthetic Oligonucleotide
tggctcttaatcagtttcgttacct

SEQ ID NO: 344 - DNA, 25 bp, Artificial Sequence
  Synthetic Oligonucleotide
caaggcatatcgatcctcataaagt

SEQ ID NO: 345 - DNA, 30 bp, Artificial Sequence
  Synthetic Oligonucleotide
tgtcccacgaataatgcgtaaattctccag

SEQ ID NO: 346 - DNA, 20 bp, Artificial Sequence
  Synthetic Oligonucleotide
gtccaggtggatgtgaaaga

SEQ ID NO: 347 - DNA, 20 bp, Artificial Sequence
  Synthetic Oligonucleotide
cggccaggatacacatctta

SEQ ID NO: 348 - DNA, 21 bp, Artificial Sequence
  Synthetic Oligonucleotide
cagcaggccctcaaggagctg

SEQ ID NO: 349 - DNA, 20 bp, Artificial Sequence
  Synthetic Oligonucleotide
acggatcacagtggaggaag

SEQ ID NO: 350 - DNA, 20 bp, Artificial Sequence
  Synthetic Oligonucleotide
ctcatccgtcgggtcatagt

SEQ ID NO: 351 - DNA, 20 bp, Artificial Sequence
  Synthetic Oligonucleotide
cgctggctcacccctacctg

SEQ ID NO: 352 - DNA, 18 bp, Artificial Sequence
  Synthetic Oligonucleotide
acccccagaccggatcag

SEQ ID NO: 353 - DNA, 23 bp, Artificial Sequence
  Synthetic Oligonucleotide
tgtagggcagacttcctcaaaca

SEQ ID NO: 354 - DNA, 23 bp, Artificial Sequence
  Synthetic Oligonucleotide
ctggccctcatgtccccttcacg

SEQ ID NO: 355 - DNA, 19 bp, Artificial Sequence
  Synthetic Oligonucleotide
cgtggtgcccctctatgac

SEQ ID NO: 356 - DNA, 19 bp, Artificial Sequence
  Synthetic Oligonucleotide
ggctagtgggcgcatgtag

SEQ ID NO: 357 - DNA, 19 bp, Artificial Sequence
  Synthetic Oligonucleotide
ctggagatgctggacgccc

SEQ ID NO: 358 - DNA, 21 bp, Artificial Sequence
  Synthetic Oligonucleotide
ggattgctcaacaaccatgct

SEQ ID NO: 359 - DNA, 24 bp, Artificial Sequence
  Synthetic Oligonucleotide
ggcattaacacttttggacgataa

SEQ ID NO: 360 - DNA, 30 bp, Artificial Sequence
  Synthetic Oligonucleotide
tctggaccctcctacctctggttcttacgt

SEQ ID NO: 361 - DNA, 24 bp, Artificial Sequence
  Synthetic Oligonucleotide
gcactttgggattctttccattat

SEQ ID NO: 362 - DNA, 24 bp, Artificial Sequence
  Synthetic Oligonucleotide
gcatgtaagaagaccctcactgaa

SEQ ID NO: 363 - DNA, 29 bp, Artificial Sequence
  Synthetic Oligonucleotide
acaacattctcggtgcctgtaacaaagaa

SEQ ID NO: 364 - DNA, 18 bp, Artificial Sequence
  Synthetic Oligonucleotide
gcctcttcctgttcgacg

SEQ ID NO: 365 - DNA, 18 bp, Artificial Sequence
  Synthetic Oligonucleotide
gctttgcccggtagctct

SEQ ID NO: 366 - DNA, 24 bp, Artificial Sequence
  Synthetic Oligonucleotide
tcgcccacctacgtactggcctac

SEQ ID NO: 367 - DNA, 25 bp, Artificial Sequence
  Synthetic Oligonucleotide
ggattgtagactgtcaccgaaattc

SEQ ID NO: 368 - DNA, 28 bp, Artificial Sequence
  Synthetic Oligonucleotide
ggctattcctcattttctctacaaagtg

SEQ ID NO: 369 - DNA, 30 bp, Artificial Sequence
  Synthetic Oligonucleotide
cctccaggaggctaccttcttcatgttcac

SEQ ID NO: 370 - DNA, 19 bp, Artificial Sequence
  Synthetic Oligonucleotide
aaggaaagggtgcctcatc

SEQ ID NO: 371 - DNA, 19 bp, Artificial Sequence
  Synthetic Oligonucleotide
gagccacaagcagcacagt

SEQ ID NO: 372 - DNA, 24 bp, Artificial Sequence
  Synthetic Oligonucleotide
catcacccacaaggacaggttgct

SEQ ID NO: 373 - DNA, 20 bp, Artificial Sequence
  Synthetic Oligonucleotide
cacgggacattcaccacatc

SEQ ID NO: 374 - DNA, 19 bp, Artificial Sequence
  Synthetic Oligonucleotide
gggtgccatccacttcaca

SEQ ID NO: 375 - DNA, 27 bp, Artificial Sequence
  Synthetic Oligonucleotide
ataaaaagacaaccaacggccgactgc

SEQ ID NO: 376 - DNA, 18 bp, Artificial Sequence
  Synthetic Oligonucleotide
ccagtggagcgcttccat

SEQ ID NO: 377 - DNA, 22 bp, Artificial Sequence
  Synthetic Oligonucleotide
ctctctgggtcgtctgaaacaa

SEQ ID NO: 378 - DNA, 23 bp, Artificial Sequence
  Synthetic Oligonucleotide
tcggccacttcatcaggacgcag

SEQ ID NO: 379 - DNA, 22 bp, Artificial Sequence
  Synthetic Oligonucleotide
ggttccagctttctgtagctgt

SEQ ID NO: 380 - DNA, 19 bp, Artificial Sequence
  Synthetic Oligonucleotide
gccgcaggttctagttgct

SEQ ID NO: 381 - DNA, 24 bp, Artificial Sequence
  Synthetic Oligonucleotide
attggtggccacaccacctcctta

SEQ ID NO: 382 - DNA, 21 bp, Artificial Sequence
  Synthetic Oligonucleotide
gcgtatgatttcccgaatgag

SEQ ID NO: 383 - DNA, 21 bp, Artificial Sequence
  Synthetic Oligonucleotide
cagtgacctcgtacccattgc

SEQ ID NO: 384 - DNA, 25 bp, Artificial Sequence
  Synthetic Oligonucleotide
atgttgatatgcccaaacttcatga

SEQ ID NO: 385 - DNA, 21 bp, Artificial Sequence
  Synthetic Oligonucleotide
cgagccctttgatgacttcct

SEQ ID NO: 386 - DNA, 18 bp, Artificial Sequence
  Synthetic Oligonucleotide
ggagcgggctgtctcaga

SEQ ID NO: 387 - DNA, 22 bp, Artificial Sequence
  Synthetic Oligonucleotide
tcccagcatcatccaggcccag

SEQ ID NO: 388 - DNA, 19 bp, Artificial Sequence
  Synthetic Oligonucleotide
ccaccccgagcaaatctgt

SEQ ID NO: 389 - DNA, 22 bp, Artificial Sequence
  Synthetic Oligonucleotide
aaatccagtccccctactttgg

SEQ ID NO: 390 - DNA, 23 bp, Artificial Sequence
  Synthetic Oligonucleotide
cctgaatcctggaggctcacgcc

SEQ ID NO: 391 - DNA, 26 bp, Artificial Sequence
  Synthetic Oligonucleotide
ggataattcagacaacaacaccatct

SEQ ID NO: 392 - DNA, 25 bp, Artificial Sequence
  Synthetic Oligonucleotide
tgaagtaatcagccacagactcaat

SEQ ID NO: 393 - DNA, 29 bp, Artificial Sequence
  Synthetic Oligonucleotide
tcaattgtaacattctcacccaggccttg

SEQ ID NO: 394 - DNA, 20 bp, Artificial Sequence
  Synthetic Oligonucleotide
gaagcgcagatcatgaagaa

SEQ ID NO: 395 - DNA, 20 bp, Artificial Sequence
  Synthetic Oligonucleotide
ctcctcagacaccactgcat

SEQ ID NO: 396 - DNA, 24 bp, Artificial Sequence
  Synthetic Oligonucleotide
ctgaagcacgacaagctggtccag

SEQ ID NO: 397 - DNA, 20 bp, Artificial Sequence
  Synthetic Oligonucleotide
cctccaactcctagcctcaa

SEQ ID NO: 398 - DNA, 18 bp, Artificial Sequence
  Synthetic Oligonucleotide
ggcgcatgcttgtaatcc

SEQ ID NO: 399 - DNA, 24 bp, Artificial Sequence
  Synthetic Oligonucleotide
tgatcctcctgtctcaacctccca

SEQ ID NO: 400 - DNA, 18 bp, Artificial Sequence
  Synthetic Oligonucleotide
gtgctggtgacgaatcca

SEQ ID NO: 401 - DNA, 20 bp, Artificial Sequence
  Synthetic Oligonucleotide
cccggcaaaaacaaataagt

SEQ ID NO: 402 - DNA, 25 bp, Artificial Sequence
  Synthetic Oligonucleotide
ttcatctcaatggaaggatcctgcc

SEQ ID NO: 403 - DNA, 20 bp, Artificial Sequence
  Synthetic Oligonucleotide
accctcgacaagaccacact

SEQ ID NO: 404 - DNA, 20 bp, Artificial Sequence
  Synthetic Oligonucleotide
tgggagttcatgggtacaga

SEQ ID NO: 405 - DNA, 24 bp, Artificial Sequence
  Synthetic Oligonucleotide
aacttcagccccagctcccaagtc

SEQ ID NO: 406 - DNA, 25 bp, Artificial Sequence
  Synthetic Oligonucleotide
aagggcaatcacagtgttaaaagaa

SEQ ID NO: 407 - DNA, 26 bp, Artificial Sequence
  Synthetic Oligonucleotide
ggagaacttcaatgaagaattttcca

SEQ ID NO: 408 - DNA, 29 bp, Artificial Sequence
  Synthetic Oligonucleotide
cataggagcagcctgcaacatttcagcat

SEQ ID NO: 409 - DNA, 20 bp, Artificial Sequence
  Synthetic Oligonucleotide
attccacccatggcaaattc

SEQ ID NO: 410 - DNA, 22 bp, Artificial Sequence
  Synthetic Oligonucleotide
gatgggatttccattgatgaca

SEQ ID NO: 411 - DNA, 22 bp, Artificial Sequence
  Synthetic Oligonucleotide
ccgttctcagccttgacggtgc

SEQ ID NO: 412 - DNA, 23 bp, Artificial Sequence
  Synthetic Oligonucleotide
caaaggagctcactgtggtgtct

SEQ ID NO: 413 - DNA, 26 bp, Artificial Sequence
  Synthetic Oligonucleotide
gagtcagaatggcttattcacagatg

SEQ ID NO: 414 - DNA, 24 bp, Artificial Sequence
  Synthetic Oligonucleotide
tgttccaaccactgaatctggacc

SEQ ID NO: 415 - DNA, 20 bp, Artificial Sequence
  Synthetic Oligonucleotide
ttgggaaatatttgggcatt

SEQ ID NO: 416 - DNA, 20 bp, Artificial Sequence
  Synthetic Oligonucleotide
agaagctagggtggttgtcc

SEQ ID NO: 417 - DNA, 26 bp, Artificial Sequence
  Synthetic Oligonucleotide
ttgggacattgtagacttggccagac

SEQ ID NO: 418 - DNA, 19 bp, Artificial Sequence
  Synthetic Oligonucleotide
gcatgggaaccatcaacca

SEQ ID NO: 419 - DNA, 21 bp, Artificial Sequence
  Synthetic Oligonucleotide
tgaggagtttgccttgattcg

SEQ ID NO: 420 - DNA, 30 bp, Artificial Sequence
  Synthetic Oligonucleotide
ccatggaccaacttcactatgtgacagagc

SEQ ID NO: 421 - DNA, 20 bp, Artificial Sequence
  Synthetic Oligonucleotide
ctgttgcaggaaggcattga

SEQ ID NO: 422 - DNA, 28 bp, Artificial Sequence
  Synthetic Oligonucleotide
gtcagtgggtctctaataaagagatgag

SEQ ID NO: 423 - DNA, 21 bp, Artificial Sequence
  Synthetic Oligonucleotide
catctcctggcccagcatgtt

SEQ ID NO: 424 - DNA, 21 bp, Artificial Sequence
  Synthetic Oligonucleotide
cgctccagacctatgatgact

SEQ ID NO: 425 - DNA, 20 bp, Artificial Sequence
  Synthetic Oligonucleotide
acagtggaaggaccaggact

SEQ ID NO: 426 - DNA, 24 bp, Artificial Sequence
  Synthetic Oligonucleotide
tgttagccaaagactgccactgca

SEQ ID NO: 427 - DNA, 18 bp, Artificial Sequence
  Synthetic Oligonucleotide
ctccgacgtggctttcca

SEQ ID NO: 428 - DNA, 23 bp, Artificial Sequence
  Synthetic Oligonucleotide
cgtaattggcagaattgatgaca

SEQ ID NO: 429 - DNA, 25 bp, Artificial Sequence
  Synthetic Oligonucleotide
tggcccacagcatctatggaatccc

SEQ ID NO: 430 - DNA, 23 bp, Artificial Sequence
  Synthetic Oligonucleotide
ggagctctgcagtaaccacagaa

SEQ ID NO: 431 - DNA, 20 bp, Artificial Sequence
  Synthetic Oligonucleotide
caatgccatccagctcttga

SEQ ID NO: 432 - DNA, 22 bp, Artificial Sequence
  Synthetic Oligonucleotide
aggccccatgctgacgtccctc

SEQ ID NO: 433 - DNA, 20 bp, Artificial Sequence
  Synthetic Oligonucleotide
ggtgaaggttgtctcttccg

SEQ ID NO: 434 - DNA, 19 bp, Artificial Sequence
  Synthetic Oligonucleotide
cagcccttccacttgtctg

SEQ ID NO: 435 - DNA, 24 bp, Artificial Sequence
  Synthetic Oligonucleotide
aagagccctgtcttcagaaggccc

SEQ ID NO: 436 - DNA, 20 bp, Artificial Sequence
  Synthetic Oligonucleotide
tacccttccatgtgctggat

SEQ ID NO: 437 - DNA, 18 bp, Artificial Sequence
  Synthetic Oligonucleotide
gctgaagaggcccaggtt

SEQ ID NO: 438 - DNA, 24 bp, Artificial Sequence
  Synthetic Oligonucleotide
cgggactccctggtcagctacatc

SEQ ID NO: 439 - DNA, 18 bp, Artificial Sequence
  Synthetic Oligonucleotide
gcttatgaccgaccccaa

SEQ ID NO: 440 - DNA, 20 bp, Artificial Sequence
  Synthetic Oligonucleotide
aaagttccaggcaacatcgt

SEQ ID NO: 441 - DNA, 24 bp, Artificial Sequence
  Synthetic Oligonucleotide
ctcatcacctggtctccggtgtgt

SEQ ID NO: 442 - DNA, 20 bp, Artificial Sequence
  Synthetic Oligonucleotide
ccatctgcatccatcttgtt

SEQ ID NO: 443 - DNA, 20 bp, Artificial Sequence
  Synthetic Oligonucleotide
ggccaccagggtattatctg

SEQ ID NO: 444 - DNA, 23 bp, Artificial Sequence
  Synthetic Oligonucleotide
ctccccacccttgagaagtgcct

SEQ ID NO: 445 - DNA, 18 bp, Artificial Sequence
  Synthetic Oligonucleotide
gacaaggacggcagcaag

SEQ ID NO: 446 - DNA, 18 bp, Artificial Sequence
  Synthetic Oligonucleotide
ttgtggcctgtctggacc

SEQ ID NO: 447 - DNA, 24 bp, Artificial Sequence
  Synthetic Oligonucleotide
ccaggagttgccaccactgttgtc

SEQ ID NO: 448 - DNA, 20 bp, Artificial Sequence
  Synthetic Oligonucleotide
gtgatcccaagcccacaata

SEQ ID NO: 449 - DNA, 18 bp, Artificial Sequence
  Synthetic Oligonucleotide
tgtggcgatcaggatgtg

SEQ ID NO: 450 - DNA, 24 bp, Artificial Sequence
  Synthetic Oligonucleotide
tcagtgggaaaaagtacaccgccc

SEQ ID NO: 451 - DNA, 20 bp, Artificial Sequence
  Synthetic Oligonucleotide
ggcccagcttgaatttttca

SEQ ID NO: 452 - DNA, 27 bp, Artificial Sequence
  Synthetic Oligonucleotide
aagctatgaggaaaagaagtacacgat

SEQ ID NO: 453 - DNA, 30 bp, Artificial Sequence
  Synthetic Oligonucleotide
tcagccactggcttctgtcataatcaggag

SEQ ID NO: 454 - DNA, 20 bp, Artificial Sequence
  Synthetic Oligonucleotide
gagaccctgctgtcccagaa

SEQ ID NO: 455 - DNA, 23 bp, Artificial Sequence
  Synthetic Oligonucleotide
ggttgtagtcagcgaaggagatc

SEQ ID NO: 456 - DNA, 26 bp, Artificial Sequence
  Synthetic Oligonucleotide
tcccacaatgaaggtcttgcctccct

SEQ ID NO: 457 - DNA, 20 bp, Artificial Sequence
  Synthetic Oligonucleotide
cccactcagtagccaagtca

SEQ ID NO: 458 - DNA, 20 bp, Artificial Sequence
  Synthetic Oligonucleotide
cacgcaggtggtatcagtct

SEQ ID NO: 459 - DNA, 27 bp, Artificial Sequence
  Synthetic Oligonucleotide
tcaagtaaacgggctgttttccaaaca

SEQ ID NO: 460 - DNA, 18 bp, Artificial Sequence
  Synthetic Oligonucleotide
tcctgtgctctggaagcc

SEQ ID NO: 461 - DNA, 21 bp, Artificial Sequence
  Synthetic Oligonucleotide
ctccacggtctcagttgatct

SEQ ID NO: 462 - DNA, 24 bp, Artificial Sequence
  Synthetic Oligonucleotide
caagaacctcccagaagggctcaa

SEQ ID NO: 463 - DNA, 20 bp, Artificial Sequence
  Synthetic Oligonucleotide
cggtgtgagaagtgcagcaa

SEQ ID NO: 464 - DNA, 19 bp, Artificial Sequence
  Synthetic Oligonucleotide
cctctcgcaagtgctccat

SEQ ID NO: 465 - DNA, 24 bp, Artificial Sequence
  Synthetic Oligonucleotide
ccagaccatagcacactcgggcac

SEQ ID NO: 466 - DNA, 24 bp, Artificial Sequence
  Synthetic Oligonucleotide
tgaacataaagtctgcaacatgga

SEQ ID NO: 467 - DNA, 28 bp, Artificial Sequence
  Synthetic Oligonucleotide
tgaggttggttactgttggtatcatata

SEQ ID NO: 468 - DNA, 24 bp, Artificial Sequence
  Synthetic Oligonucleotide
ttgcactgcacaggccacattcac

SEQ ID NO: 469 - DNA, 24 bp, Artificial Sequence
  Synthetic Oligonucleotide
tccaggatgttaggaactgtgaag

SEQ ID NO: 470 - DNA, 22 bp, Artificial Sequence
  Synthetic Oligonucleotide
gcgtgtctgcgtagtagctgtt

SEQ ID NO: 471 - DNA, 24 bp, Artificial Sequence
  Synthetic Oligonucleotide
agtcgctggtttcatgcccttcca

SEQ ID NO: 472 - DNA, 19 bp, Artificial Sequence
  Synthetic Oligonucleotide
ggacgaatacgaccccact

SEQ ID NO: 473 - DNA, 18 bp, Artificial Sequence
  Synthetic Oligonucleotide
gcacgtctccccatcaat

SEQ ID NO: 474 - DNA, 24 bp, Artificial Sequence
  Synthetic Oligonucleotide
accacctgcttccggtaggaatcc

SEQ ID NO: 475 - DNA, 19 bp, Artificial Sequence
  Synthetic Oligonucleotide
ctgctgcgacagtccacta

SEQ ID NO: 476 - DNA, 18 bp, Artificial Sequence
  Synthetic Oligonucleotide
caggttcgctctgggaag

SEQ ID NO: 477 - DNA, 24 bp, Artificial Sequence
  Synthetic Oligonucleotide
agagtgactcccgttgtcccaagg

SEQ ID NO: 478 - DNA, 19 bp, Artificial Sequence
  Synthetic Oligonucleotide
ggtccgcttcgtctttcga

SEQ ID NO: 479 - DNA, 19 bp, Artificial Sequence
  Synthetic Oligonucleotide
gcacaggttcgctctggaa

SEQ ID NO: 480 - DNA, 20 bp, Artificial Sequence
  Synthetic Oligonucleotide
tgactcccgcggtcccaagg

SEQ ID NO: 481 - DNA, 19 bp, Artificial Sequence
  Synthetic Oligonucleotide
gcaggtgtgattgctggac

SEQ ID NO: 482 - DNA, 18 bp, Artificial Sequence
  Synthetic Oligonucleotide
accataggcaatggcagc

SEQ ID NO: 483 - DNA, 24 bp, Artificial Sequence
  Synthetic Oligonucleotide
aagaatcatcaatgagcccacggc

SEQ ID NO: 484 - DNA, 24 bp, Artificial Sequence
  Synthetic Oligonucleotide
ggctagtagaactggatcccaaca

SEQ ID NO: 485 - DNA, 20 bp, Artificial Sequence
  Synthetic Oligonucleotide
ggtctgcccaaatgcttttc

SEQ ID NO: 486 - DNA, 30 bp, Artificial Sequence
  Synthetic Oligonucleotide
taattagacctaggcctcagctgcactgcc

SEQ ID NO: 487 - DNA, 19 bp, Artificial Sequence
  Synthetic Oligonucleotide
ggccactaaagatgctggc

SEQ ID NO: 488 - DNA, 18 bp, Artificial Sequence
  Synthetic Oligonucleotide
agcagctgtgggctcatt

SEQ ID NO: 489 - DNA, 24 bp, Artificial Sequence
  Synthetic Oligonucleotide
atcacccgaagcacattcagtcca

SEQ ID NO: 490 - DNA, 19 bp, Artificial Sequence
  Synthetic Oligonucleotide
ccgactggaggagcataaa

SEQ ID NO: 491 - DNA, 19 bp, Artificial Sequence
  Synthetic Oligonucleotide
atgctggctgactctgctc

SEQ ID NO: 492 - DNA, 24 bp, Artificial Sequence
  Synthetic Oligonucleotide
cgcacttttctgagcagacgtcca

SEQ ID NO: 493 - DNA, 20 bp, Artificial Sequence
  Synthetic Oligonucleotide
caaaaggcagaggctgataa

SEQ ID NO: 494 - DNA, 20 bp, Artificial Sequence
  Synthetic Oligonucleotide
agcgcagtttcataaagcaa

SEQ ID NO: 495 - DNA, 27 bp, Artificial Sequence
  Synthetic Oligonucleotide
tgaccagatccttcacagacttgtcgt

SEQ ID NO: 496 - DNA, 19 bp, Artificial Sequence
  Synthetic Oligonucleotide
agaaccgcaaggtgagcaa

SEQ ID NO: 497 - DNA, 21 bp, Artificial Sequence
  Synthetic Oligonucleotide
tccaactgaaggtccctgatg

SEQ ID NO: 498 - DNA, 26 bp, Artificial Sequence
  Synthetic Oligonucleotide
tggagattctccagcacgtcatcgac

SEQ ID NO: 499 - DNA, 20 bp, Artificial Sequence
  Synthetic Oligonucleotide
cacgcagaaaaccacacttc

SEQ ID NO: 500 - DNA, 20 bp, Artificial Sequence
  Synthetic Oligonucleotide
catgttcctccttgtgcatc

SEQ ID NO: 501 - DNA, 24 bp, Artificial Sequence
  Synthetic Oligonucleotide
caacacttccttccccaaagccag

SEQ ID NO: 502 - DNA, 21 bp, Artificial Sequence
  Synthetic Oligonucleotide
gcatggtagccgaagatttca

SEQ ID NO: 503 - DNA, 30 bp, Artificial Sequence
  Synthetic Oligonucleotide
tttccggtaatagtctgtctcatagatatc

SEQ ID NO: 504 - DNA, 28 bp, Artificial Sequence
  Synthetic Oligonucleotide
cgcgtcataccaaaatctccgattttga

SEQ ID NO: 505 - DNA, 19 bp, Artificial Sequence
  Synthetic Oligonucleotide
gtggacagcaccatgaaca

SEQ ID NO: 506 - DNA, 20 bp, Artificial Sequence
  Synthetic Oligonucleotide
ccttcatacccgacttgagg

SEQ ID NO: 507 - DNA, 20 bp, Artificial Sequence
  Synthetic Oligonucleotide
cttccggccagcactgcctc

SEQ ID NO: 508 - DNA, 17 bp, Artificial Sequence
  Synthetic Oligonucleotide
acgcaccgggtgtctga

SEQ ID NO: 509 - DNA, 24 bp, Artificial Sequence
  Synthetic Oligonucleotide
tgccctttcttgatgatgattatc

SEQ ID NO: 510 - DNA, 24 bp, Artificial Sequence
  Synthetic Oligonucleotide
cccaagttccaccccctccattca

SEQ ID NO: 511 - DNA, 22 bp, Artificial Sequence
  Synthetic Oligonucleotide
tggacaagtacgggatgaagct

SEQ ID NO: 512 - DNA, 21 bp, Artificial Sequence
  Synthetic Oligonucleotide
cgaaggtgtggcactgaaagt

SEQ ID NO: 513 - DNA, 24 bp, Artificial Sequence
  Synthetic Oligonucleotide
cccgtcaacgtactccatgcctgg

SEQ ID NO: 514 - DNA, 19 bp, Artificial Sequence
  Synthetic Oligonucleotide
gcggtgattcggaaattcg

SEQ ID NO: 515 - DNA, 19 bp, Artificial Sequence
  Synthetic Oligonucleotide
ctctcctgggcacctgctt

SEQ ID NO: 516 - DNA, 23 bp, Artificial Sequence
  Synthetic Oligonucleotide
ctctggtcctcatccaggtgcgc

SEQ ID NO: 517 - DNA, 26 bp, Artificial Sequence
  Synthetic Oligonucleotide
aaggaaccatctcactgtgtgtaaac

SEQ ID NO: 518 - DNA, 20 bp, Artificial Sequence
  Synthetic Oligonucleotide
atcaggaaggctgccaagag

SEQ ID NO: 519 - DNA, 22 bp, Artificial Sequence
  Synthetic Oligonucleotide
tgacttccaagctggccgtggc

SEQ ID NO: 520 - DNA, 20 bp, Artificial Sequence
  Synthetic Oligonucleotide
tctgcgtggttcctttctca

SEQ ID NO: 521 - DNA, 25 bp, Artificial Sequence
  Synthetic Oligonucleotide
ttgaaggatgtttattaggcaacgt

SEQ ID NO: 522 - DNA, 26 bp, Artificial Sequence
  Synthetic Oligonucleotide
cgcttctgactgctgattctcccgtt

SEQ ID NO: 523 - DNA, 20 bp, Artificial Sequence
  Synthetic Oligonucleotide
cctgaaccttccaaagatgg

SEQ ID NO: 524 - DNA, 20 bp, Artificial Sequence
  Synthetic Oligonucleotide
accaggcaagtctcctcatt

SEQ ID NO: 525 - DNA, 27 bp, Artificial Sequence
  Synthetic Oligonucleotide
ccagattggaagcatccatctttttca

SEQ ID NO: 526 - DNA, 20 bp, Artificial Sequence
  Synthetic Oligonucleotide
ccgctccgtcactgatgtct

SEQ ID NO: 527 - DNA, 21 bp, Artificial Sequence
  Synthetic Oligonucleotide
gcaaggtcagggcaaagagta

SEQ ID NO: 528 - DNA, 24 bp, Artificial Sequence
  Synthetic Oligonucleotide
cctgctgaacctagccttggccga

SEQ ID NO: 529 - DNA, 20 bp, Artificial Sequence
  Synthetic Oligonucleotide
ctcaggattttctcgcatcc

SEQ ID NO: 530 - DNA, 19 bp, Artificial Sequence
  Synthetic Oligonucleotide
aggagcaggtggagactgg

SEQ ID NO: 531 - DNA, 24 bp, Artificial Sequence
  Synthetic Oligonucleotide
atgtgctcccagtgctaggtgcct

SEQ ID NO: 532 - DNA, 20 bp, Artificial Sequence
  Synthetic Oligonucleotide
agccatcactctcagtgcag

SEQ ID NO: 533 - DNA, 20 bp, Artificial Sequence
  Synthetic Oligonucleotide
actgcagagtcagggtctcc

SEQ ID NO: 534 - DNA, 22 bp, Artificial Sequence
  Synthetic Oligonucleotide
caggtcctatcgtggcccctga

SEQ ID NO: 535 - DNA, 21 bp, Artificial Sequence
  Synthetic Oligonucleotide
gccaggatactggagtccatc

SEQ ID NO: 536 - DNA, 18 bp, Artificial Sequence
  Synthetic Oligonucleotide
cttgacccttggggtcct

SEQ ID NO: 537 - DNA, 23 bp, Artificial Sequence
  Synthetic Oligonucleotide
tgagctccctgatggctacaccc

SEQ ID NO: 538 - DNA, 18 bp, Artificial Sequence
  Synthetic Oligonucleotide
ggatggagttcgcctcct

SEQ ID NO: 539 - DNA, 20 bp, Artificial Sequence
  Synthetic Oligonucleotide
cgctccatggacttgatctt

SEQ ID NO: 540 - DNA, 24 bp, Artificial Sequence
  Synthetic Oligonucleotide
cgtgatcacagacctgacccagct

SEQ ID NO: 541 - DNA, 20 bp, Artificial Sequence
  Synthetic Oligonucleotide
ccacagctcaccttctgtca

SEQ ID NO: 542 - DNA, 20 bp, Artificial Sequence
  Synthetic Oligonucleotide
cctcagtgccagtctcttcc

SEQ ID NO: 543 - DNA, 20 bp, Artificial Sequence
  Synthetic Oligonucleotide
tccatcccagctccagccag

SEQ ID NO: 544 - DNA, 20 bp, Artificial Sequence
  Synthetic Oligonucleotide
tcagaattggatttggctca

SEQ ID NO: 545 - DNA, 20 bp, Artificial Sequence
  Synthetic Oligonucleotide
cctgagcttagctggtgttg

SEQ ID NO: 546 - DNA, 30 bp, Artificial Sequence
  Synthetic Oligonucleotide
tgctaatgtaaggcatcacagtcttttcca

SEQ ID NO: 547 - DNA, 26 bp, Artificial Sequence
  Synthetic Oligonucleotide
tgaggaagaaggagaggaatactaat

SEQ ID NO: 548 - DNA, 21 bp, Artificial Sequence
  Synthetic Oligonucleotide
ctgaaattctgggagcatgac

SEQ ID NO: 549 - DNA, 24 bp, Artificial Sequence
  Synthetic Oligonucleotide
tatccattccttttggccctgcag

SEQ ID NO: 550 - DNA, 20 bp, Artificial Sequence
  Synthetic Oligonucleotide
gaggacgaaggcctctacac

SEQ ID NO: 551 - DNA, 20 bp, Artificial Sequence
  Synthetic Oligonucleotide
aaaaatgcctccacttttgc

SEQ ID NO: 552 - DNA, 24 bp, Artificial Sequence
  Synthetic Oligonucleotide
caggcatgcagtgttcttggctgt

SEQ ID NO: 553 - DNA, 19 bp, Artificial Sequence
  Synthetic Oligonucleotide
cggactttgggtgcgactt

SEQ ID NO: 554 - DNA, 24 bp, Artificial Sequence
  Synthetic Oligonucleotide
ttacaactcttccactgggacgat

SEQ ID NO: 555 - DNA, 23 bp, Artificial Sequence
  Synthetic Oligonucleotide
ccacttgtcgaaccaccgctcgt

SEQ ID NO: 556 - DNA, 20 bp, Artificial Sequence
  Synthetic Oligonucleotide
tggaggttgtaagccaatgt

SEQ ID NO: 557 - DNA, 20 bp, Artificial Sequence
  Synthetic Oligonucleotide
tgccttacgtccatctgatt

SEQ ID NO: 558 - DNA, 28 bp, Artificial Sequence
  Synthetic Oligonucleotide
cagtgatgtctgaacttgaagcctcaca

SEQ ID NO: 559 - DNA, 20 bp, Artificial Sequence
  Synthetic Oligonucleotide
ctaaggcacttgctggaagg

SEQ ID NO: 560 - DNA, 18 bp, Artificial Sequence
  Synthetic Oligonucleotide
tcttcccagctcctgtgg

SEQ ID NO: 561 - DNA, 24 bp, Artificial Sequence
  Synthetic Oligonucleotide
tccataggcaagcacactggcatt

SEQ ID NO: 562 - DNA, 25 bp, Artificial Sequence
  Synthetic Oligonucleotide
aattcctgctccaaaagaaagtctt

SEQ ID NO: 563 - DNA, 20 bp, Artificial Sequence
  Synthetic Oligonucleotide
cgtgatgcgaagctctgaga

SEQ ID NO: 564 - DNA, 22 bp, Artificial Sequence
  Synthetic Oligonucleotide
aagccgctccactcgcatgtcc

SEQ ID NO: 565 - DNA, 20 bp, Artificial Sequence
  Synthetic Oligonucleotide
ccacagggttgaagaaccag

SEQ ID NO: 566 - DNA, 20 bp, Artificial Sequence
  Synthetic Oligonucleotide
cacctgatgtgccagacttc

SEQ ID NO: 567 - DNA, 24 bp, Artificial Sequence
  Synthetic Oligonucleotide
agccagttcctgctgttcctgtcc

SEQ ID NO: 568 - DNA, 18 bp, Artificial Sequence
  Synthetic Oligonucleotide
gcccagaggctccatcgt

SEQ ID NO: 569 - DNA, 23 bp, Artificial Sequence
  Synthetic Oligonucleotide
cagaggtttgaacagtgcagaca

SEQ ID NO: 570 - DNA, 23 bp, Artificial Sequence
  Synthetic Oligonucleotide
cctcttcctccccagtcggctga

SEQ ID NO: 571 - DNA, 20 bp, Artificial Sequence
  Synthetic Oligonucleotide
caaacagagggtggcagaag

SEQ ID NO: 572 - DNA, 18 bp, Artificial Sequence
  Synthetic Oligonucleotide
gaggctctcacggctcct

SEQ ID NO: 573 - DNA, 24 bp, Artificial Sequence
  Synthetic Oligonucleotide
cgcttctccatgttctcagggtca

SEQ ID NO: 574 - DNA, 18 bp, Artificial Sequence
  Synthetic Oligonucleotide
agccgaggctttgttgaa

SEQ ID NO: 575 - DNA, 19 bp, Artificial Sequence
  Synthetic Oligonucleotide
tgggctatgagcacagctt

SEQ ID NO: 576 - DNA, 24 bp, Artificial Sequence
  Synthetic Oligonucleotide
ttcatatccagtaccggcgatcgg

SEQ ID NO: 577 - DNA, 20 bp, Artificial Sequence
  Synthetic Oligonucleotide
atgtgccagtgagcttgagt

SEQ ID NO: 578 - DNA, 20 bp, Artificial Sequence
  Synthetic Oligonucleotide
tgagcccctggttaacagta

SEQ ID NO: 579 - DNA, 24 bp, Artificial Sequence
  Synthetic Oligonucleotide
ccttggagaaacacaagcacctgc

SEQ ID NO: 580 - DNA, 20 bp, Artificial Sequence
  Synthetic Oligonucleotide
ggcctgctgagatcaaagac

SEQ ID NO: 581 - DNA, 20 bp, Artificial Sequence
  Synthetic Oligonucleotide
gtccactgtggctgtgagaa

SEQ ID NO: 582 - DNA, 26 bp, Artificial Sequence
  Synthetic Oligonucleotide
tgttcctcaggtcctcaatggtcttg

SEQ ID NO: 583 - DNA, 21 bp, Artificial Sequence
  Synthetic Oligonucleotide
cgaggattggttcttcagcaa

SEQ ID NO: 584 - DNA, 24 bp, Artificial Sequence
  Synthetic Oligonucleotide
cacctcgcggttcagttcctctgt

SEQ ID NO: 585 - DNA, 22 bp, Artificial Sequence
  Synthetic Oligonucleotide
actctgcaccagctcactgttg

SEQ ID NO: 586 - DNA, 21 bp, Artificial Sequence
  Synthetic Oligonucleotide
tgagcggcagaatcaggagta

SEQ ID NO: 587 - DNA, 19 bp, Artificial Sequence
  Synthetic Oligonucleotide
tgcggtaggtggcaatctc

SEQ ID NO: 588 - DNA, 24 bp, Artificial Sequence
  Synthetic Oligonucleotide
ctcatggacatcaagtcgcggctg

SEQ ID NO: 589 - DNA, 20 bp, Artificial Sequence
  Synthetic Oligonucleotide
tcagtggagaaggagttgga

SEQ ID NO: 590 - DNA, 20 bp, Artificial Sequence
  Synthetic Oligonucleotide
tgccatatccagaggaaaca

SEQ ID NO: 591 - DNA, 28 bp, Artificial Sequence
  Synthetic Oligonucleotide
ccagtcaacatctctgttgtcacaagca

SEQ ID NO: 592 - DNA, 18 bp, Artificial Sequence
  Synthetic Oligonucleotide
cttgctggccaatgccta

SEQ ID NO: 593 - DNA, 18 bp, Artificial Sequence
  Synthetic Oligonucleotide
tgattgtccgcagtcagg

SEQ ID NO: 594 - DNA, 24 bp, Artificial Sequence
  Synthetic Oligonucleotide
atctacgttgtccagctgccagcc

SEQ ID NO: 595 - DNA, 21 bp, Artificial Sequence
  Synthetic Oligonucleotide
actcaagcggaaattgaagca

SEQ ID NO: 596 - DNA, 21 bp, Artificial Sequence
  Synthetic Oligonucleotide
actccctgaagccgagacact

SEQ ID NO: 597 - DNA, 28 bp, Artificial Sequence
  Synthetic Oligonucleotide
aggtcttatcagcacagtctccgcctcc

SEQ ID NO: 598 - DNA, 18 bp, Artificial Sequence
  Synthetic Oligonucleotide
agcgatgaagatggtcgc

SEQ ID NO: 599 - DNA, 18 bp, Artificial Sequence
  Synthetic Oligonucleotide
gacatggcagcacaagca

SEQ ID NO: 600 - DNA, 24 bp, Artificial Sequence
  Synthetic Oligonucleotide
ctggacgcggttctactccaacag

SEQ ID NO: 601 - DNA, 20 bp, Artificial Sequence
  Synthetic Oligonucleotide
gcttcaggtgttgtgactgc

SEQ ID NO: 602 - DNA, 20 bp, Artificial Sequence
  Synthetic Oligonucleotide
aagagctgcccatccttctc

SEQ ID NO: 603 - DNA, 24 bp, Artificial Sequence
  Synthetic Oligonucleotide
tgcctccctgtcgcaccagtacta

SEQ ID NO: 604 - DNA, 18 bp, Artificial Sequence
  Synthetic Oligonucleotide
ctttgggccaggaggaat

SEQ ID NO: 605 - DNA, 18 bp, Artificial Sequence
  Synthetic Oligonucleotide
ctcccacaatccactgcc

SEQ ID NO: 606 - DNA, 24 bp, Artificial Sequence
  Synthetic Oligonucleotide
actcgaatccacccaggaactccc

SEQ ID NO: 607 - DNA, 20 bp, Artificial Sequence
  Synthetic Oligonucleotide
agaagctgtccctgcaagag

SEQ ID NO: 608 - DNA, 20 bp, Artificial Sequence
  Synthetic Oligonucleotide
agccgtaccagctcagactt

SEQ ID NO: 609 - DNA, 25 bp, Artificial Sequence
  Synthetic Oligonucleotide
catgttcttcacaatcgctgcatcc

SEQ ID NO: 610 - DNA, 18 bp, Artificial Sequence
  Synthetic Oligonucleotide
ccgggagcagggaatcac

SEQ ID NO: 611 - DNA, 20 bp, Artificial Sequence
  Synthetic Oligonucleotide
atgctgttgatgccgaatga

SEQ ID NO: 612 - DNA, 19 bp, Artificial Sequence
  Synthetic Oligonucleotide
cggccacgatttcggcgct

SEQ ID NO: 613 - DNA, 20 bp, Artificial Sequence
  Synthetic Oligonucleotide
ctgtcatgtggcagaccttc

SEQ ID NO: 614 - DNA, 20 bp, Artificial Sequence
  Synthetic Oligonucleotide
taaatgtcactggtgcctgg

SEQ ID NO: 615 - DNA, 24 bp, Artificial Sequence
  Synthetic Oligonucleotide
cgaaccacggcttgggaagactac

SEQ ID NO: 616 - DNA, 19 bp, Artificial Sequence
  Synthetic Oligonucleotide
gcccagtggagaaattcgt

SEQ ID NO: 617 - DNA, 19 bp, Artificial Sequence
  Synthetic Oligonucleotide
gcgagtctgagctgatgga

SEQ ID NO: 618 - DNA, 24 bp, Artificial Sequence
  Synthetic Oligonucleotide
tttgagatcacccagcctccactg

SEQ ID NO: 619 - DNA, 20 bp, Artificial Sequence
  Synthetic Oligonucleotide
cctcagaaattgccaggact

SEQ ID NO: 620 - DNA, 24 bp, Artificial Sequence
  Synthetic Oligonucleotide
ttcccgtgatcttcagcaaagcct

SEQ ID NO: 621 - DNA, 20 bp, Artificial Sequence
  Synthetic Oligonucleotide
ccaaagaccagctgcaagta

SEQ ID NO: 622 - DNA, 20 bp, Artificial Sequence
  Synthetic Oligonucleotide
aggactccagcaaccaagaa

SEQ ID NO: 623 - DNA, 20 bp, Artificial Sequence
  Synthetic Oligonucleotide
gagtgctgcttggaactcag

SEQ ID NO: 624 - DNA, 24 bp, Artificial Sequence
  Synthetic Oligonucleotide
caagcaccttccctgacctggagt

SEQ ID NO: 625 - DNA, 18 bp, Artificial Sequence
  Synthetic Oligonucleotide
cggaccaccaggtcagag

SEQ ID NO: 626 - DNA, 20 bp, Artificial Sequence
  Synthetic Oligonucleotide
caggggtagtgggtgttgag

SEQ ID NO: 627 - DNA, 24 bp, Artificial Sequence
  Synthetic Oligonucleotide
ccactcttccctgctctgcgaatt

SEQ ID NO: 628 - DNA, 20 bp, Artificial Sequence
  Synthetic Oligonucleotide
gccctccaatgtccttatca

SEQ ID NO: 629 - DNA, 21 bp, Artificial Sequence
  Synthetic Oligonucleotide
gtagccactgatgccaaagtc

SEQ ID NO: 630 - DNA, 24 bp, Artificial Sequence
  Synthetic Oligonucleotide
cacatcttcacatggccctccttg

SEQ ID NO: 631 - DNA, 18 bp, Artificial Sequence
  Synthetic Oligonucleotide
gccggtcaggcacacaag

SEQ ID NO: 632 - DNA, 23 bp, Artificial Sequence
  Synthetic Oligonucleotide
gcagcatacacacaacaaaatgg

SEQ ID NO: 633 - DNA, 24 bp, Artificial Sequence
  Synthetic Oligonucleotide
accaaccagtccacgctccaaggg

SEQ ID NO: 634 - DNA, 18 bp, Artificial Sequence
  Synthetic Oligonucleotide
ccctcaaccggcaaatcc

SEQ ID NO: 635 - DNA, 19 bp, Artificial Sequence
  Synthetic Oligonucleotide
cgtccatgccctgaattca

SEQ ID NO: 636 - DNA, 23 bp, Artificial Sequence
  Synthetic Oligonucleotide
tggcgagtgcagtgagcagctcc

SEQ ID NO: 637 - DNA, 23 bp, Artificial Sequence
  Synthetic Oligonucleotide
tgagtggaaaagcctgacctatg

SEQ ID NO: 638 - DNA, 23 bp, Artificial Sequence
  Synthetic Oligonucleotide
ggactccatctcttcttggtcaa

SEQ ID NO: 639 - DNA, 27 bp, Artificial Sequence
  Synthetic Oligonucleotide
tgaagtcatcagctttgtgccaccacc

SEQ ID NO: 640 - DNA, 22 bp, Artificial Sequence
  Synthetic Oligonucleotide
caacacccgtacatcaatgtct

SEQ ID NO: 641 - DNA, 22 bp, Artificial Sequence
  Synthetic Oligonucleotide
tcatctaactgcttgtcaggga

SEQ ID NO: 642 - DNA, 24 bp, Artificial Sequence
  Synthetic Oligonucleotide
ctgaagcagaagctccaccaccaa

SEQ ID NO: 643 - DNA, 20 bp, Artificial Sequence
  Synthetic Oligonucleotide
gaccttggaacctttggaac

SEQ ID NO: 644 - DNA, 20 bp, Artificial Sequence
  Synthetic Oligonucleotide
cctaggcctatgagggttca

SEQ ID NO: 645 - DNA, 26 bp, Artificial Sequence
  Synthetic Oligonucleotide
cagccctgtaagacctgttgacagca

SEQ ID NO: 646 - DNA, 18 bp, Artificial Sequence
  Synthetic Oligonucleotide
cacaagctgaccttccgc

SEQ ID NO: 647 - DNA, 20 bp, Artificial Sequence
  Synthetic Oligonucleotide
acttgtacacgatctccgcc

SEQ ID NO: 648 - DNA, 24 bp, Artificial Sequence
  Synthetic Oligonucleotide
agaacgccaaagccaagacagacc

SEQ ID NO: 649 - DNA, 23 bp, Artificial Sequence
  Synthetic Oligonucleotide
cagatggccactttgagaacatt

SEQ ID NO: 650 - DNA, 22 bp, Artificial Sequence
  Synthetic Oligonucleotide
ggcagcattaaccacaaggatt

SEQ ID NO: 651 - DNA, 28 bp, Artificial Sequence
  Synthetic Oligonucleotide
agctgacaacagtgtgaacgaccagacc

SEQ ID NO: 652 - DNA, 20 bp, Artificial Sequence
  Synthetic Oligonucleotide
cttcggaaactggacatcaa

SEQ ID NO: 653 - DNA, 20 bp, Artificial Sequence
  Synthetic Oligonucleotide
gtcgctgaaaacatggatca

SEQ ID NO: 654 - DNA, 26 bp, Artificial Sequence
  Synthetic Oligonucleotide
tcactcgagacaacgatttcacatcg

SEQ ID NO: 655 - DNA, 21 bp, Artificial Sequence
  Synthetic Oligonucleotide
gacttttgcccgctacctttc

SEQ ID NO: 656 - DNA, 26 bp, Artificial Sequence
  Synthetic Oligonucleotide
gccactaactgcttcagtatgaagag

SEQ ID NO: 657 - DNA, 24 bp, Artificial Sequence
  Synthetic Oligonucleotide
acagctcattgttgtcacgccgga

SEQ ID NO: 658 - DNA, 24 bp, Artificial Sequence
  Synthetic Oligonucleotide
tgatggtcctatgtgtcacattca

SEQ ID NO: 659 - DNA, 20 bp, Artificial Sequence
  Synthetic Oligonucleotide
tgggacaggaaacacaccaa

SEQ ID NO: 660 - DNA, 30 bp, Artificial Sequence
  Synthetic Oligonucleotide
caggtttcataccaacacaggcttcagcac

SEQ ID NO: 661 - DNA, 19 bp, Artificial Sequence
  Synthetic Oligonucleotide
cgctcagccagatgcaatc

SEQ ID NO: 662 - DNA, 25 bp, Artificial Sequence
  Synthetic Oligonucleotide
gcactgagatcttcctattggtgaa

SEQ ID NO: 663 - DNA, 21 bp, Artificial Sequence
  Synthetic Oligonucleotide
tgccccagtcacctgctgtta

SEQ ID NO: 664 - DNA, 20 bp, Artificial Sequence
  Synthetic Oligonucleotide
gtgaaatgaaacgcaccaca

SEQ ID NO: 665 - DNA, 20 bp, Artificial Sequence
  Synthetic Oligonucleotide
gaccctgctcacaaccagac

SEQ ID NO: 666 - DNA, 20 bp, Artificial Sequence
  Synthetic Oligonucleotide
cagccctttggggaagctgg

SEQ ID NO: 667 - DNA, 19 bp, Artificial Sequence
  Synthetic Oligonucleotide
ccaacgcttgccaaatcct

SEQ ID NO: 668 - DNA, 24 bp, Artificial Sequence
  Synthetic Oligonucleotide
acggtagtgacagcatcaaaactc

SEQ ID NO: 669 - DNA, 24 bp, Artificial Sequence
  Synthetic Oligonucleotide
aaccagctctctgtgaccccaatt

SEQ ID NO: 670 - DNA, 20 bp, Artificial Sequence
  Synthetic Oligonucleotide
ccatgatggagaggcagaca

SEQ ID NO: 671 - DNA, 21 bp, Artificial Sequence
  Synthetic Oligonucleotide
ggagtccgtccttaccgtcaa

SEQ ID NO: 672 - DNA, 24 bp, Artificial Sequence
  Synthetic Oligonucleotide
ctgggagcatggcgatggataccc

SEQ ID NO: 673 - DNA, 20 bp, Artificial Sequence
  Synthetic Oligonucleotide
gagaaccaatctcaccgaca

SEQ ID NO: 674 - DNA, 20 bp, Artificial Sequence
  Synthetic Oligonucleotide
cacccgagtgtaaccatagc

SEQ ID NO: 675 - DNA, 24 bp, Artificial Sequence
  Synthetic Oligonucleotide
acaggtattcctctgccagctgcc

SEQ ID NO: 676 - DNA, 18 bp, Artificial Sequence
  Synthetic Oligonucleotide
gccatgctggctcagtct

SEQ ID NO: 677 - DNA, 20 bp, Artificial Sequence
  Synthetic Oligonucleotide
cacccagacccacctaactg

SEQ ID NO: 678 - DNA, 24 bp, Artificial Sequence
  Synthetic Oligonucleotide
cactagctgatgtggcccacagct

SEQ ID NO: 679 - DNA, 18 bp, Artificial Sequence
  Synthetic Oligonucleotide
tcatggtgcccgtcaatg

SEQ ID NO: 680 - DNA, 23 bp, Artificial Sequence
  Synthetic Oligonucleotide
cgattgtctttgctcttcatgtg

SEQ ID NO: 681 - DNA, 29 bp, Artificial Sequence
  Synthetic Oligonucleotide
acctgatacgtcttggtcttcatcgccat

SEQ ID NO: 682 - DNA, 20 bp, Artificial Sequence
  Synthetic Oligonucleotide
aggggatgacttggacacat

SEQ ID NO: 683 - DNA, 20 bp, Artificial Sequence
  Synthetic Oligonucleotide
aaaactgcatggctttgtca

SEQ ID NO: 684 - DNA, 25 bp, Artificial Sequence
  Synthetic Oligonucleotide
ctgccattcgacatgactgcaattt

SEQ ID NO: 685 - DNA, 22 bp, Artificial Sequence
  Synthetic Oligonucleotide
tcatcctggcgatctacttcct

SEQ ID NO: 686 - DNA, 20 bp, Artificial Sequence
  Synthetic Oligonucleotide
ccgttgagtggaatcagcaa

SEQ ID NO: 687 - DNA, 26 bp, Artificial Sequence
  Synthetic Oligonucleotide
tctgtcctggctggagtcgctttcat

SEQ ID NO: 688 - DNA, 21 bp, Artificial Sequence
  Synthetic Oligonucleotide
tgattaccatcatggctcaga

SEQ ID NO: 689 - DNA, 20 bp, Artificial Sequence
  Synthetic Oligonucleotide
cttgtgaaaatgccatccac

SEQ ID NO: 690 - DNA, 24 bp, Artificial Sequence
  Synthetic Oligonucleotide
tcccaattgtcgcttcttctgcag

SEQ ID NO: 691 - DNA, 20 bp, Artificial Sequence
  Synthetic Oligonucleotide
ggccaggatctgtggtggta

SEQ ID NO: 692 - DNA, 20 bp, Artificial Sequence
  Synthetic Oligonucleotide
ctccacgtcgtggacattga

SEQ ID NO: 693 - DNA, 23 bp, Artificial Sequence
  Synthetic Oligonucleotide
ctctggccttccgagaaggtacc

SEQ ID NO: 694 - DNA, 23 bp, Artificial Sequence
  Synthetic Oligonucleotide
gaaggaatgggaatcagtcatga

SEQ ID NO: 695 - DNA, 26 bp, Artificial Sequence
  Synthetic Oligonucleotide
gtctattagagtcagatccgggacat

SEQ ID NO: 696 - DNA, 23 bp, Artificial Sequence
  Synthetic Oligonucleotide
tcaccctggagatcagctcccga

SEQ ID NO: 697 - DNA, 18 bp, Artificial Sequence
  Synthetic Oligonucleotide
gccgagatcgccaagatg

SEQ ID NO: 698 - DNA, 27 bp, Artificial Sequence
  Synthetic Oligonucleotide
cttttgatggtagagttccagtgattc

SEQ ID NO: 699 - DNA, 24 bp, Artificial Sequence
  Synthetic Oligonucleotide
cagcattgtctgtcctccctggca

SEQ ID NO: 700 - DNA, 27 bp, Artificial Sequence
  Synthetic Oligonucleotide
cggtacttctcagggctaatatatacg

SEQ ID NO: 701 - DNA, 21 bp, Artificial Sequence
  Synthetic Oligonucleotide
ccgagtagatgggcaggtgtt

SEQ ID NO: 702 - DNA, 26 bp, Artificial Sequence
  Synthetic Oligonucleotide
ctcttctgcgtggtggtcaaccccta

SEQ ID NO: 703 - DNA, 18 bp, Artificial Sequence
  Synthetic Oligonucleotide
gtgaggcagcgcgactct

SEQ ID NO: 704 - DNA, 23 bp, Artificial Sequence
  Synthetic Oligonucleotide
tgccaatggtgtacaacacttca

SEQ ID NO: 705 - DNA, 21 bp, Artificial Sequence
  Synthetic Oligonucleotide
tgccttcccgggctgaggact

SEQ ID NO: 706 - DNA, 20 bp, Artificial Sequence
  Synthetic Oligonucleotide
cagaccaaggagatggacct

SEQ ID NO: 707 - DNA, 18 bp, Artificial Sequence
  Synthetic Oligonucleotide
agctgccagtgctatccg

SEQ ID NO: 708 - DNA, 24 bp, Artificial Sequence
  Synthetic Oligonucleotide
aagctgtaaacatgagccgcacca

SEQ ID NO: 709 - DNA, 19 bp, Artificial Sequence
  Synthetic Oligonucleotide
ctgccgggatggcttctat

SEQ ID NO: 710 - DNA, 22 bp, Artificial Sequence
  Synthetic Oligonucleotide
ccaggttctggaaactgtggat

SEQ ID NO: 711 - DNA, 21 bp, Artificial Sequence
  Synthetic Oligonucleotide
ctgagctctgcccggaccgct

SEQ ID NO: 712 - DNA, 18 bp, Artificial Sequence
  Synthetic Oligonucleotide
cactgacacccgcaacac

SEQ ID NO: 713 - DNA, 19 bp, Artificial Sequence
  Synthetic Oligonucleotide
ggctgcaatctctctgctg

SEQ ID NO: 714 - DNA, 24 bp, Artificial Sequence
  Synthetic Oligonucleotide
aaccacagagtccgaaccatgggt

SEQ ID NO: 715 - DNA, 19 bp, Artificial Sequence
  Synthetic Oligonucleotide
ctgcttctttcccgagctt

SEQ ID NO: 716 - DNA, 19 bp, Artificial Sequence
  Synthetic Oligonucleotide
agcaggaatgtagctgcca

SEQ ID NO: 717 - DNA, 24 bp, Artificial Sequence
  Synthetic Oligonucleotide
acttcgttatccgcgatgcgtttc

SEQ ID NO: 718 - DNA, 20 bp, Artificial Sequence
  Synthetic Oligonucleotide
ggctgtggctgaggctgtag

SEQ ID NO: 719 - DNA, 21 bp, Artificial Sequence
  Synthetic Oligonucleotide
ggagcattcgaggtcaaatca

SEQ ID NO: 720 - DNA, 28 bp, Artificial Sequence
  Synthetic Oligonucleotide
ttcccagagtgtctcacctccagcagag

SEQ ID NO: 721 - DNA, 20 bp, Artificial Sequence
  Synthetic Oligonucleotide
cggacctgaatgtaatgcaa

SEQ ID NO: 722 - DNA, 20 bp, Artificial Sequence
  Synthetic Oligonucleotide
ctttgccaggtgactaagca

SEQ ID NO: 723 - DNA, 28 bp, Artificial Sequence
  Synthetic Oligonucleotide
aatgaacagaacaagcaaaatgaccagc

SEQ ID NO: 724 - DNA, 20 bp, Artificial Sequence
  Synthetic Oligonucleotide
caaaggaagagcaacggaag

SEQ ID NO: 725 - DNA, 19 bp, Artificial Sequence
  Synthetic Oligonucleotide
attcccaaaacctttgctt

SEQ ID NO: 726 - DNA, 24 bp, Artificial Sequence
  Synthetic Oligonucleotide
ttctcctttcgttcttgctcgcgt

SEQ ID NO: 727 - DNA, 21 bp, Artificial Sequence
  Synthetic Oligonucleotide
tggagactctcagggtcgaaa

SEQ ID NO: 728 - DNA, 22 bp, Artificial Sequence
  Synthetic Oligonucleotide
ggcgtttggagtggtagaaatc

SEQ ID NO: 729 - DNA, 20 bp, Artificial Sequence
  Synthetic Oligonucleotide
cggcggcagaccagcatgac

SEQ ID NO: 730 - DNA, 21 bp, Artificial Sequence
  Synthetic Oligonucleotide
cggtggaccacgaagagttaa

SEQ ID NO: 731 - DNA, 19 bp, Artificial Sequence
  Synthetic Oligonucleotide
ggctcgcctcttccatgtc

SEQ ID NO: 732 - DNA, 23 bp, Artificial Sequence
  Synthetic Oligonucleotide
ccgggacttggagaagcactgca

SEQ ID NO: 733 - DNA, 20 bp, Artificial Sequence
  Synthetic Oligonucleotide
tcactaccagctgacatccg

SEQ ID NO: 734 - DNA, 20 bp, Artificial Sequence
  Synthetic Oligonucleotide
agatggggctattgagggtc

SEQ ID NO: 735 - DNA, 24 bp, Artificial Sequence
  Synthetic Oligonucleotide
cttctccaggtagccctcaggcag

SEQ ID NO: 736 - DNA, 20 bp, Artificial Sequence
  Synthetic Oligonucleotide
ccagctctccttccagctac

SEQ ID NO: 737 - DNA, 20 bp, Artificial Sequence
  Synthetic Oligonucleotide
gggtggctctcacttagctc

SEQ ID NO: 738 - DNA, 24 bp, Artificial Sequence
  Synthetic Oligonucleotide
atcaatgtccctgtccgagtgctg

SEQ ID NO: 739 - DNA, 19 bp, Artificial Sequence
  Synthetic Oligonucleotide
gagaagcacgccatgaaac

SEQ ID NO: 740 - DNA, 20 bp, Artificial Sequence
  Synthetic Oligonucleotide
ggctgtgagctgctgaatct

SEQ ID NO: 741 - DNA, 24 bp, Artificial Sequence
  Synthetic Oligonucleotide
tgactcatcatttccatgacggcc

SEQ ID NO: 742 - DNA, 24 bp, Artificial Sequence
  Synthetic Oligonucleotide
agagccagttgctgtagaactcaa

SEQ ID NO: 743 - DNA, 21 bp, Artificial Sequence
  Synthetic Oligonucleotide
ctgggcctacacagtccttca

SEQ ID NO: 744 - DNA, 27 bp, Artificial Sequence
  Synthetic Oligonucleotide
tctctgctgggcaaggatgttctgttc

SEQ ID NO: 745 - DNA, 20 bp, Artificial Sequence
  Synthetic Oligonucleotide
gacattgtggtaggggaagg

SEQ ID NO: 746 - DNA, 20 bp, Artificial Sequence
  Synthetic Oligonucleotide
cggcagtcaaagataattgg

SEQ ID NO: 747 - DNA, 26 bp, Artificial Sequence
  Synthetic Oligonucleotide
tcattttctcatcccgtgggtacaga

SEQ ID NO: 748 - DNA, 23 bp, Artificial Sequence
  Synthetic Oligonucleotide
cacactagcattttctccgcata

SEQ ID NO: 749 - DNA, 21 bp, Artificial Sequence
  Synthetic Oligonucleotide
ataccaatcaccgcacaaacc

SEQ ID NO: 750 - DNA, 30 bp, Artificial Sequence
  Synthetic Oligonucleotide
tgcgctgtgactggacttaacaaatagcct

SEQ ID NO: 751 - DNA, 20 bp, Artificial Sequence
  Synthetic Oligonucleotide
ctgctcaaggacaccgtcta

SEQ ID NO: 752 - DNA, 18 bp, Artificial Sequence
  Synthetic Oligonucleotide
ggatccactctggagggc

SEQ ID NO: 753 - DNA, 24 bp, Artificial Sequence
  Synthetic Oligonucleotide
tacactcgggtcccatcgaagtcc

SEQ ID NO: 754 - DNA, 19 bp, Artificial Sequence
  Synthetic Oligonucleotide
tggggacattccctttgag

SEQ ID NO: 755 - DNA, 18 bp, Artificial Sequence
  Synthetic Oligonucleotide
gacatgggctgggaagtg

SEQ ID NO: 756 - DNA, 24 bp, Artificial Sequence
  Synthetic Oligonucleotide
cagcttccagaatctcctggtccc

SEQ ID NO: 757 - DNA, 20 bp, Artificial Sequence
  Synthetic Oligonucleotide
cccatggatgctcctctgaa

SEQ ID NO: 758 - DNA, 20 bp, Artificial Sequence
  Synthetic Oligonucleotide
ccggtggctaccagacattg

SEQ ID NO: 759 - DNA, 22 bp, Artificial Sequence
  Synthetic Oligonucleotide
cattgactgccgaggccccatg

SEQ ID NO: 760 - DNA, 18 bp, Artificial Sequence
  Synthetic Oligonucleotide
ccaagttctgggtggtgg

SEQ ID NO: 761 - DNA, 19 bp, Artificial Sequence
  Synthetic Oligonucleotide
gtgaacgccagtccatgtt

SEQ ID NO: 762 - DNA, 24 bp, Artificial Sequence
  Synthetic Oligonucleotide
ccagacccacttctacctgggcag

SEQ ID NO: 763 - DNA, 26 bp, Artificial Sequence
  Synthetic Oligonucleotide
aatgaatacagtattcccaagcacat

SEQ ID NO: 764 - DNA, 23 bp, Artificial Sequence
  Synthetic Oligonucleotide
tgtctgaagcatcttctggatga

SEQ ID NO: 765 - DNA, 18 bp, Artificial Sequence
  Synthetic Oligonucleotide
aaccccgtggccgcctcc

SEQ ID NO: 766 - DNA, 21 bp, Artificial Sequence
  Synthetic Oligonucleotide
cttacggttttcgtggagaag

SEQ ID NO: 767 - DNA, 20 bp, Artificial Sequence
  Synthetic Oligonucleotide
agcagccgttcttgttgtaa

SEQ ID NO: 768 - DNA, 29 bp, Artificial Sequence
  Synthetic Oligonucleotide
cctcagctatacaacaaattgaccccaag

SEQ ID NO: 769 - DNA, 20 bp, Artificial Sequence
  Synthetic Oligonucleotide
gatgtggactgccattcaaa

SEQ ID NO: 770 - DNA, 20 bp, Artificial Sequence
  Synthetic Oligonucleotide
tgcgagattagttggctgag

SEQ ID NO: 771 - DNA, 28 bp, Artificial Sequence
  Synthetic Oligonucleotide
tcgaaatttacatccggtatcttcctgg

SEQ ID NO: 772 - DNA, 19 bp, Artificial Sequence
  Synthetic Oligonucleotide
ccacataccgtccagccta

SEQ ID NO: 773 - DNA, 18 bp, Artificial Sequence
  Synthetic Oligonucleotide
gaggtcatgtgcgggagt

SEQ ID NO: 774 - DNA, 24 bp, Artificial Sequence
  Synthetic Oligonucleotide
ccgctcctcttcttcgttctccag

SEQ ID NO: 775 - DNA, 20 bp, Artificial Sequence
  Synthetic Oligonucleotide
gcaatcatggaacttgacga

SEQ ID NO: 776 - DNA, 18 bp, Artificial Sequence
  Synthetic Oligonucleotide
atgtggctcgcctctacg

SEQ ID NO: 777 - DNA, 24 bp, Artificial Sequence
  Synthetic Oligonucleotide
tttcttgcagtttgacccagcacc

SEQ ID NO: 778 - DNA, 20 bp, Artificial Sequence
  Synthetic Oligonucleotide
gcatcaggctgtcattatgg

SEQ ID NO: 779 - DNA, 20 bp, Artificial Sequence
  Synthetic Oligonucleotide
agtagttgtgctgcccttcc

SEQ ID NO: 780 - DNA, 28 bp, Artificial Sequence
  Synthetic Oligonucleotide
tgtccttacctgtgggagctgtaaggtc

SEQ ID NO: 781 - DNA, 19 bp, Artificial Sequence
  Synthetic Oligonucleotide
aggactgggacccatgaac

SEQ ID NO: 782 - DNA, 20 bp, Artificial Sequence
  Synthetic Oligonucleotide
cccataatcctgagcaatgg

SEQ ID NO: 783 - DNA, 24 bp, Artificial Sequence
  Synthetic Oligonucleotide
tcctttggtatcagacccgaagcg

SEQ ID NO: 784 - DNA, 20 bp, Artificial Sequence
  Synthetic Oligonucleotide
ggtgtccttcgccagatcac

SEQ ID NO: 785 - DNA, 18 bp, Artificial Sequence
  Synthetic Oligonucleotide
cagccgcagagcctcatc

SEQ ID NO: 786 - DNA, 26 bp, Artificial Sequence
  Synthetic Oligonucleotide
ttaatgatttgcctgtgggacgctcc

SEQ ID NO: 787 - DNA, 21 bp, Artificial Sequence
  Synthetic Oligonucleotide
caagcaatgcgtcatcaatgt

SEQ ID NO: 788 - DNA, 20 bp, Artificial Sequence
  Synthetic Oligonucleotide
gtaaatccgccccctcttct

SEQ ID NO: 789 - DNA, 25 bp, Artificial Sequence
  Synthetic Oligonucleotide
cagcctctgcggaatggatcacact

SEQ ID NO: 790 - DNA, 20 bp, Artificial Sequence
  Synthetic Oligonucleotide
ctgacacttgccgcagagaa

SEQ ID NO: 791 - DNA, 20 bp, Artificial Sequence
  Synthetic Oligonucleotide
aggtggtccttggtctggaa

SEQ ID NO: 792 - DNA, 26 bp, Artificial Sequence
  Synthetic Oligonucleotide
ccctttctcacccacctcatctgcac

SEQ ID NO: 793 - DNA, 18 bp, Artificial Sequence
  Synthetic Oligonucleotide
gggttctagacgcccctc

SEQ ID NO: 794 - DNA, 21 bp, Artificial Sequence
  Synthetic Oligonucleotide
ggacggctgtagaggctgtat

SEQ ID NO: 795 - DNA, 24 bp, Artificial Sequence
  Synthetic Oligonucleotide
caagcgttcctggccttctgaact

SEQ ID NO: 796 - DNA, 21 bp, Artificial Sequence
  Synthetic Oligonucleotide
ctccacctatgagcgtctgtc

SEQ ID NO: 797 - DNA, 20 bp, Artificial Sequence
  Synthetic Oligonucleotide
cacactttccctccttttgg

SEQ ID NO: 798 - DNA, 24 bp, Artificial Sequence
  Synthetic Oligonucleotide
tcctgttaacatcccaagcccaca

SEQ ID NO: 799 - DNA, 19 bp, Artificial Sequence
  Synthetic Oligonucleotide
gccctcccagtgtgcaaat

SEQ ID NO: 800 - DNA, 25 bp, Artificial Sequence
  Synthetic Oligonucleotide
cgtcgatggtattaggatagaagca

SEQ ID NO: 801 - DNA, 23 bp, Artificial Sequence
  Synthetic Oligonucleotide
tgctgtttcgacgacaccgttcg

SEQ ID NO: 802 - DNA, 25 bp, Artificial Sequence
  Synthetic Oligonucleotide
tggctaagtgaagatgacaatcatg

SEQ ID NO: 803 - DNA, 25 bp, Artificial Sequence
  Synthetic Oligonucleotide
tgcacatatcattacaccagttcgt

SEQ ID NO: 804 - DNA, 29 bp, Artificial Sequence
  Synthetic Oligonucleotide
cctttccagctttacagtgaattgctgca

SEQ ID NO: 805 - DNA, 23 bp, Artificial Sequence
  Synthetic Oligonucleotide
cgcttgcctaactcatactttcc

SEQ ID NO: 806 - DNA, 20 bp, Artificial Sequence
  Synthetic Oligonucleotide
ccattcagactgcgccactt

SEQ ID NO: 807 - DNA, 19 bp, Artificial Sequence
  Synthetic Oligonucleotide
tccacgcagcgtggcactg

SEQ ID NO: 808 - DNA, 28 bp, Artificial Sequence
  Synthetic Oligonucleotide
ggctactctgatctatgttgataaggaa

SEQ ID NO: 809 - DNA, 20 bp, Artificial Sequence
  Synthetic Oligonucleotide
gcttcagcccatccttagca

SEQ ID NO: 810 - DNA, 22 bp, Artificial Sequence
  Synthetic Oligonucleotide
cacacgggtgcctggttctcca

SEQ ID NO: 811 - DNA, 18 bp, Artificial Sequence
  Synthetic Oligonucleotide
gggacactgcgggacaag

SEQ ID NO: 812 - DNA, 18 bp, Artificial Sequence
  Synthetic Oligonucleotide
gcccatggcgtctctgaa

SEQ ID NO: 813 - DNA, 25 bp, Artificial Sequence
  Synthetic Oligonucleotide
cggttccggagtctcaccactgcat

SEQ ID NO: 814 - DNA, 19 bp, Artificial Sequence
  Synthetic Oligonucleotide
ctgaaggaccctacgctcg

SEQ ID NO: 815 - DNA, 19 bp, Artificial Sequence
  Synthetic Oligonucleotide
atgcaaagccagtgtgctc

SEQ ID NO: 816 - DNA, 24 bp, Artificial Sequence
  Synthetic Oligonucleotide
cttctcaaagtgaggtgccaggcc

SEQ ID NO: 817 - DNA, 21 bp, Artificial Sequence
  Synthetic Oligonucleotide
gcgacagctcctctagttcca

SEQ ID NO: 818 - DNA, 19 bp, Artificial Sequence
  Synthetic Oligonucleotide
ttcccgaagtctccgcccg

SEQ ID NO: 819 - DNA, 22 bp, Artificial Sequence
  Synthetic Oligonucleotide
ggaacaccagcttgaatttcct

SEQ ID NO: 820 - DNA, 20 bp, Artificial Sequence
  Synthetic Oligonucleotide
gaggagtggtgacagtctgc

SEQ ID NO: 821 - DNA, 20 bp, Artificial Sequence
  Synthetic Oligonucleotide
gctgcagaaatcaaagtcca

SEQ ID NO: 822 - DNA, 27 bp, Artificial Sequence
  Synthetic Oligonucleotide
tcaatacacaggaacctgaggagaccc

SEQ ID NO: 823 - DNA, 23 bp, Artificial Sequence
  Synthetic Oligonucleotide
agctagcctcagtgacacacatg

SEQ ID NO: 824 - DNA, 18 bp, Artificial Sequence
  Synthetic Oligonucleotide
ccggatctgacggctgtt

SEQ ID NO: 825 - DNA, 24 bp, Artificial Sequence
  Synthetic Oligonucleotide
acacaacgtcggcagtgcaacctg

SEQ ID NO: 826 - DNA, 20 bp, Artificial Sequence
  Synthetic Oligonucleotide
cgtcgtatgcgagagtctgt

SEQ ID NO: 827 - DNA, 20 bp, Artificial Sequence
  Synthetic Oligonucleotide
tgaaggcgtgaggtgtagaa

SEQ ID NO: 828 - DNA, 26 bp, Artificial Sequence
  Synthetic Oligonucleotide
tccaggatgcctgttagttctcagca

SEQ ID NO: 829 - DNA, 26 bp, Artificial Sequence
  Synthetic Oligonucleotide
ggtgtcagatataaatgtgcaaatgc

SEQ ID NO: 830 - DNA, 24 bp, Artificial Sequence
  Synthetic Oligonucleotide
ttcgatattgccagcagctataaa

SEQ ID NO: 831 - DNA, 28 bp, Artificial Sequence
  Synthetic Oligonucleotide
tgctgtcctgtcggtctcagtacgttca

SEQ ID NO: 832 - DNA, 20 bp, Artificial Sequence
  Synthetic Oligonucleotide
tgtggatgctggattgattt

SEQ ID NO: 833 - DNA, 20 bp, Artificial Sequence
  Synthetic Oligonucleotide
aagcagcacttcctggtctt

SEQ ID NO: 834 - DNA, 25 bp, Artificial Sequence
  Synthetic Oligonucleotide
ccactggtgcagctgctaaatagca

SEQ ID NO: 835 - DNA, 20 bp, Artificial Sequence
  Synthetic Oligonucleotide
agtgggagacacctgacctt

SEQ ID NO: 836 - DNA, 20 bp, Artificial Sequence
  Synthetic Oligonucleotide
tgatctgggcattgtactcc

SEQ ID NO: 837 - DNA, 29 bp, Artificial Sequence
  Synthetic Oligonucleotide
ttgatcttctgctcaatctcagcttgaga

SEQ ID NO: 838 - DNA, 20 bp, Artificial Sequence
  Synthetic Oligonucleotide
cgaagcccttacaagtttcc

SEQ ID NO: 839 - DNA, 20 bp, Artificial Sequence
  Synthetic Oligonucleotide
ggactcttcaggggtgaaat

SEQ ID NO: 840 - DNA, 24 bp, Artificial Sequence
  Synthetic Oligonucleotide
cccttacggattcctggagggaac

SEQ ID NO: 841 - DNA, 20 bp, Artificial Sequence
  Synthetic Oligonucleotide
cccagtgtacgaggaacaag

SEQ ID NO: 842 - DNA, 20 bp, Artificial Sequence
  Synthetic Oligonucleotide
ttagcgaggaaggagttgct

SEQ ID NO: 843 - DNA, 24 bp, Artificial Sequence
  Synthetic Oligonucleotide
acagaccgagatctccaaccggac

SEQ ID NO: 844 - DNA, 18 bp, Artificial Sequence
  Synthetic Oligonucleotide
gggctgggtgagaatgtg

SEQ ID NO: 845 - DNA, 19 bp, Artificial Sequence
  Synthetic Oligonucleotide
cacaacatcctccggaatg

SEQ ID NO: 846 - DNA, 24 bp, Artificial Sequence
  Synthetic Oligonucleotide
ataccagggccggcttcttcctct

SEQ ID NO: 847 - DNA, 18 bp, Artificial Sequence
  Synthetic Oligonucleotide
cctacaagtcctggggca

SEQ ID NO: 848 - DNA, 20 bp, Artificial Sequence
  Synthetic Oligonucleotide
tgaggtcaggctcacacagt

SEQ ID NO: 849 - DNA, 24 bp, Artificial Sequence
  Synthetic Oligonucleotide
tggagccccaagcagtgttaatcc

SEQ ID NO: 850 - DNA, 20 bp, Artificial Sequence
  Synthetic Oligonucleotide
gcgaggagctctacttgctc

SEQ ID NO: 851 - DNA, 18 bp, Artificial Sequence
  Synthetic Oligonucleotide
gccctgctgaacaccact

SEQ ID NO: 852 - DNA, 24 bp, Artificial Sequence
  Synthetic Oligonucleotide
tgtcctctttctgcaccttgtcgc

SEQ ID NO: 853 - DNA, 21 bp, Artificial Sequence
  Synthetic Oligonucleotide
aagcatgaacaggacttgacc

SEQ ID NO: 854 - DNA, 18 bp, Artificial Sequence
  Synthetic Oligonucleotide
cctccccaagtcagttgc

SEQ ID NO: 855 - DNA, 24 bp, Artificial Sequence
  Synthetic Oligonucleotide
ctttccaacccctggggaagacat

SEQ ID NO: 856 - DNA, 18 bp, Artificial Sequence
  Synthetic Oligonucleotide
cccgttcggtctgaggaa

SEQ ID NO: 857 - DNA, 22 bp, Artificial Sequence
  Synthetic Oligonucleotide
gagcactcaaggtagccaaagg

SEQ ID NO: 858 - DNA, 19 bp, Artificial Sequence
  Synthetic Oligonucleotide
tccggttcgccatgtcccg

SEQ ID NO: 859 - DNA, 20 bp, Artificial Sequence
  Synthetic Oligonucleotide
ccagacgagcgattagaagc

SEQ ID NO: 860 - DNA, 20 bp, Artificial Sequence
  Synthetic Oligonucleotide
tcctcctcttcctcctcctc

SEQ ID NO: 861 - DNA, 23 bp, Artificial Sequence
  Synthetic Oligonucleotide
tgtgaggtgaatgatttggggga

SEQ ID NO: 862 - DNA, 22 bp, Artificial Sequence
  Synthetic Oligonucleotide
tgtgcacataggaatgagcttc

SEQ ID NO: 863 - DNA, 20 bp, Artificial Sequence
  Synthetic Oligonucleotide
gtttagcacgcaattgctca

SEQ ID NO: 864 - DNA, 24 bp, Artificial Sequence
  Synthetic Oligonucleotide
tcactctctttgctggccaactgc

SEQ ID NO: 865 - DNA, 19 bp, Artificial Sequence
  Synthetic Oligonucleotide
gatctggcactgtggttcc

SEQ ID NO: 866 - DNA, 20 bp, Artificial Sequence
  Synthetic Oligonucleotide
tgacagcggaagtggtattg

SEQ ID NO: 867 - DNA, 22 bp, Artificial Sequence
  Synthetic Oligonucleotide
caccgccagccactgtcttcat

SEQ ID NO: 868 - DNA, 24 bp, Artificial Sequence
  Synthetic Oligonucleotide
ccattctatcatcaacgggtacaa

SEQ ID NO: 869 - DNA, 23 bp, Artificial Sequence
  Synthetic Oligonucleotide
tcagcaagtgggaaggtgtaatc

SEQ ID NO: 870 - DNA, 25 bp, Artificial Sequence
  Synthetic Oligonucleotide
tctccacagacaaggccaggactcg

SEQ ID NO: 871 - DNA, 19 bp, Artificial Sequence
  Synthetic Oligonucleotide
ctgtcttcctgttggccct

SEQ ID NO: 872 - DNA, 20 bp, Artificial Sequence
  Synthetic Oligonucleotide
gtgaggtagtgagtgggcgt

SEQ ID NO: 873 - DNA, 24 bp, Artificial Sequence
  Synthetic Oligonucleotide
acaatcatagccagcacctgggct

SEQ ID NO: 874 - DNA, 25 bp, Artificial Sequence
  Synthetic Oligonucleotide
gctcattatgaaaaacatcccaaac

SEQ ID NO: 875 - DNA, 26 bp, Artificial Sequence
  Synthetic Oligonucleotide
aagaaacagaagttgtctggctttct

SEQ ID NO: 876 - DNA, 24 bp, Artificial Sequence
  Synthetic Oligonucleotide
cacaccaaccaataatttcgcatt

SEQ ID NO: 877 - DNA, 20 bp, Artificial Sequence
  Synthetic Oligonucleotide
gctctgttgtgtcctgttgc

SEQ ID NO: 878 - DNA, 21 bp, Artificial Sequence
  Synthetic Oligonucleotide
gtacggagaagccacttcaca

SEQ ID NO: 879 - DNA, 23 bp, Artificial Sequence
  Synthetic Oligonucleotide
tcagtcacaggaaggccagagcc

SEQ ID NO: 880 - DNA, 18 bp, Artificial Sequence
  Synthetic Oligonucleotide
cgaggagatgcctgtgga

SEQ ID NO: 881 - DNA, 18 bp, Artificial Sequence
  Synthetic Oligonucleotide
caacgccctggtcactct

SEQ ID NO: 882 - DNA, 24 bp, Artificial Sequence
  Synthetic Oligonucleotide
ctgttccacagcaagctctgcctc

SEQ ID NO: 883 - DNA, 20 bp, Artificial Sequence
  Synthetic Oligonucleotide
acaccaaaatgccatctcaa

SEQ ID NO: 884 - DNA, 20 bp, Artificial Sequence
  Synthetic Oligonucleotide
tttatccccagcgaatttgt

SEQ ID NO: 885 - DNA, 24 bp, Artificial Sequence
  Synthetic Oligonucleotide
cacgccatggaaaccatgatgttt

SEQ ID NO: 886 - DNA, 18 bp, Artificial Sequence
  Synthetic Oligonucleotide
cttctgagcccgtctccc

SEQ ID NO: 887 - DNA, 18 bp, Artificial Sequence
  Synthetic Oligonucleotide
gagagctccccttccgag

SEQ ID NO: 888 - DNA, 24 bp, Artificial Sequence
  Synthetic Oligonucleotide
cgcttctggagcagcttcctcaac

SEQ ID NO: 889 - DNA, 20 bp, Artificial Sequence
  Synthetic Oligonucleotide
cgcgagcccctcattataca

SEQ ID NO: 890 - DNA, 19 bp, Artificial Sequence
  Synthetic Oligonucleotide
cactcgccgttgacatcct

SEQ ID NO: 891 - DNA, 22 bp, Artificial Sequence
  Synthetic Oligonucleotide
ctccccacagcgcatcgaggaa

SEQ ID NO: 892 - DNA, 20 bp, Artificial Sequence
  Synthetic Oligonucleotide
gagagagccagtctgatcca

SEQ ID NO: 893 - DNA, 19 bp, Artificial Sequence
  Synthetic Oligonucleotide
aggctgccatgtcctcata

SEQ ID NO: 894 - DNA, 22 bp, Artificial Sequence
  Synthetic Oligonucleotide
ctgctctgccagcttggccttc

SEQ ID NO: 895 - DNA, 23 bp, Artificial Sequence
  Synthetic Oligonucleotide
cagtggagaccagttgggtagtg

SEQ ID NO: 896 - DNA, 20 bp, Artificial Sequence
  Synthetic Oligonucleotide
ccttgaagagcgtcccatca

SEQ ID NO: 897 - DNA, 28 bp, Artificial Sequence
  Synthetic Oligonucleotide
cacacatgcagagcttgtagcgtaccca

SEQ ID NO: 898 - DNA, 18 bp, Artificial Sequence
  Synthetic Oligonucleotide
tccgcaagacacctcctg

SEQ ID NO: 899 - DNA, 18 bp, Artificial Sequence
  Synthetic Oligonucleotide
tgaagtcatccttggccc

SEQ ID NO: 900 - DNA, 23 bp, Artificial Sequence
  Synthetic Oligonucleotide
tgtcctgtccttctgcaggaggc

SEQ ID NO: 901 - DNA, 20 bp, Artificial Sequence
  Synthetic Oligonucleotide
tgcattcgttggtttctctt

SEQ ID NO: 902 - DNA, 20 bp, Artificial Sequence
  Synthetic Oligonucleotide
tttctgaatggcaaactgct

SEQ ID NO: 903 - DNA, 30 bp, Artificial Sequence
  Synthetic Oligonucleotide
tgcacctccttcagaagacttatttttgtg

SEQ ID NO: 904 - DNA, 20 bp, Artificial Sequence
  Synthetic Oligonucleotide
ccaacaccttcttggcttct

SEQ ID NO: 905 - DNA, 20 bp, Artificial Sequence
  Synthetic Oligonucleotide
ctgttatcccaacccaaacc

SEQ ID NO: 906 - DNA, 24 bp, Artificial Sequence
  Synthetic Oligonucleotide
cctgtgttcttgctgagcaccctc

SEQ ID NO: 907 - DNA, 20 bp, Artificial Sequence
  Synthetic Oligonucleotide
agctggggtgtctgtttcat

SEQ ID NO: 908 - DNA, 20 bp, Artificial Sequence
  Synthetic Oligonucleotide
acagcaaggcgagcataaat

SEQ ID NO: 909 - DNA, 23 bp, Artificial Sequence
  Synthetic Oligonucleotide
cctgacttcaggtcaagggatgg

SEQ ID NO: 910 - DNA, 18 bp, Artificial Sequence
  Synthetic Oligonucleotide
gtggggagcccatcatct

SEQ ID NO: 911 - DNA, 20 bp, Artificial Sequence
  Synthetic Oligonucleotide
ccagtccacactgagtgcat

SEQ ID NO: 912 - DNA, 24 bp, Artificial Sequence
  Synthetic Oligonucleotide
ccaagccatcacaggctctgcata

SEQ ID NO: 913 - DNA, 20 bp, Artificial Sequence
  Synthetic Oligonucleotide
tctgccagtgctgaattctt

SEQ ID NO: 914 - DNA, 20 bp, Artificial Sequence
  Synthetic Oligonucleotide
ttcgaaccttagcagcttcc

SEQ ID NO: 915 - DNA, 24 bp, Artificial Sequence
  Synthetic Oligonucleotide
tgctgacattgccctggctcctat

SEQ ID NO: 916 - DNA, 20 bp, Artificial Sequence
  Synthetic Oligonucleotide
cccaactcaggtccttggta

SEQ ID NO: 917 - DNA, 18 bp, Artificial Sequence
  Synthetic Oligonucleotide
caagagggtcaccccaag

SEQ ID NO: 918 - DNA, 24 bp, Artificial Sequence
  Synthetic Oligonucleotide
atcctgcaagccaatcccagtcat

SEQ ID NO: 919 - DNA, 22 bp, Artificial Sequence
  Synthetic Oligonucleotide
agatgcatacttggaagcacag

SEQ ID NO: 920 - DNA, 22 bp, Artificial Sequence
  Synthetic Oligonucleotide
aacctaggaccaggtaaccaca

SEQ ID NO: 921 - DNA, 24 bp, Artificial Sequence
  Synthetic Oligonucleotide
catatcacactgggaggcaatgca

SEQ ID NO: 922 - DNA, 18 bp, Artificial Sequence
  Synthetic Oligonucleotide
gcgcagaggccagttaaa

SEQ ID NO: 923 - DNA, 18 bp, Artificial Sequence
  Synthetic Oligonucleotide
agctgagctgtgggttgc

SEQ ID NO: 924 - DNA, 24 bp, Artificial Sequence
  Synthetic Oligonucleotide
agatcacctcctcgaacccactcc

SEQ ID NO: 925 - DNA, 19 bp, Artificial Sequence
  Synthetic Oligonucleotide
ggctggccaaacataagca

SEQ ID NO: 926 - DNA, 26 bp, Artificial Sequence
  Synthetic Oligonucleotide
tccttgtcacagtatttacagctgaa

SEQ ID NO: 927 - DNA, 29 bp, Artificial Sequence
  Synthetic Oligonucleotide
ctgcactgcgatgcccagtctagaaaatc

SEQ ID NO: 928 - DNA, 21 bp, Artificial Sequence
  Synthetic Oligonucleotide
agtgacaaatgttggaggagc

SEQ ID NO: 929 - DNA, 19 bp, Artificial Sequence
  Synthetic Oligonucleotide
ccctccactgtcttctggg

SEQ ID NO: 930 - DNA, 24 bp, Artificial Sequence
  Synthetic Oligonucleotide
tactgctgtcacacccgtcaccac

SEQ ID NO: 931 - DNA, 19 bp, Artificial Sequence
  Synthetic Oligonucleotide
acccaccatggatgtcttc

SEQ ID NO: 932 - DNA, 19 bp, Artificial Sequence
  Synthetic Oligonucleotide
cctgcttggtcttttccac

SEQ ID NO: 933 - DNA, 24 bp, Artificial Sequence
  Synthetic Oligonucleotide
aagaagggcttctccatcgccaag

SEQ ID NO: 934 - DNA, 20 bp, Artificial Sequence
  Synthetic Oligonucleotide
tgaagagaggcatgttggag

SEQ ID NO: 935 - DNA, 20 bp, Artificial Sequence
  Synthetic Oligonucleotide
aatagacacatcggccacac

SEQ ID NO: 936 - DNA, 24 bp, Artificial Sequence
  Synthetic Oligonucleotide
tttgtcagcagtcacattgcccaa

SEQ ID NO: 937 - DNA, 24 bp, Artificial Sequence
  Synthetic Oligonucleotide
atacagcaccaatggaaagatcac

SEQ ID NO: 938 - DNA, 24 bp, Artificial Sequence
  Synthetic Oligonucleotide
tgtctgtaagagccctcagtttga

SEQ ID NO: 939 - DNA, 24 bp, Artificial Sequence
  Synthetic Oligonucleotide
ttcgacgactacatcgcctgctgc

SEQ ID NO: 940 - DNA, 20 bp, Artificial Sequence
  Synthetic Oligonucleotide
gggctcagctttcagaagtg

SEQ ID NO: 941 - DNA, 20 bp, Artificial Sequence
  Synthetic Oligonucleotide
acatgttcagctggtccaca

SEQ ID NO: 942 - DNA, 25 bp, Artificial Sequence
  Synthetic Oligonucleotide
tggcagttttcttctgtcaccaaaa

SEQ ID NO: 943 - DNA, 20 bp, Artificial Sequence
  Synthetic Oligonucleotide
tcacatgccactttggtgtt

SEQ ID NO: 944 - DNA, 20 bp, Artificial Sequence
  Synthetic Oligonucleotide
cttgcaggaagcggctatac

SEQ ID NO: 945 - DNA, 22 bp, Artificial Sequence
  Synthetic Oligonucleotide
tcctgggagagattgaccagca

SEQ ID NO: 946 - DNA, 19 bp, Artificial Sequence
  Synthetic Oligonucleotide
caagagggactcggactgc

SEQ ID NO: 947 - DNA, 21 bp, Artificial Sequence
  Synthetic Oligonucleotide
caggtcagtggagagattggt

SEQ ID NO: 948 - DNA, 24 bp, Artificial Sequence
  Synthetic Oligonucleotide
cctctgcacctctgagagcatgga

SEQ ID NO: 949 - DNA, 18 bp, Artificial Sequence
  Synthetic Oligonucleotide
ggactcggagacgctgtg

SEQ ID NO: 950 - DNA, 20 bp, Artificial Sequence
  Synthetic Oligonucleotide
gggatccttcgcaacttctt

SEQ ID NO: 951 - DNA, 24 bp, Artificial Sequence
  Synthetic Oligonucleotide
ttcttgaggatcttgacggccctc

SEQ ID NO: 952 - DNA, 20 bp, Artificial Sequence
  Synthetic Oligonucleotide
catcttccaggaggaccact

SEQ ID NO: 953 - DNA, 20 bp, Artificial Sequence
  Synthetic Oligonucleotide
tccgaccttcaatcatttca

SEQ ID NO: 954 - DNA, 24 bp, Artificial Sequence
  Synthetic Oligonucleotide
ctctgtggcaccctggactacctg

SEQ ID NO: 955 - DNA, 20 bp, Artificial Sequence
  Synthetic Oligonucleotide
aatacccaacgcacaaatga

SEQ ID NO: 956 - DNA, 20 bp, Artificial Sequence
  Synthetic Oligonucleotide
ggagacaatgcaaaccacac

SEQ ID NO: 957 - DNA, 23 bp, Artificial Sequence
  Synthetic Oligonucleotide
cacgttctctgccccgtttcttg

SEQ ID NO: 958 - DNA, 20 bp, Artificial Sequence
  Synthetic Oligonucleotide
cctggaggctgcaacatacc

SEQ ID NO: 959 - DNA, 23 bp, Artificial Sequence
  Synthetic Oligonucleotide
tacaatggctttggaggatagca

SEQ ID NO: 960 - DNA, 25 bp, Artificial Sequence
  Synthetic Oligonucleotide
atcctcctgaagcccttttcgcagc

SEQ ID NO: 961 - DNA, 20 bp, Artificial Sequence
  Synthetic Oligonucleotide
tgttttgattcccgggctta

SEQ ID NO: 962 - DNA, 24 bp, Artificial Sequence
  Synthetic Oligonucleotide
caaagctgtcagctctagcaaaag

SEQ ID NO: 963 - DNA, 28 bp, Artificial Sequence
  Synthetic Oligonucleotide
tgccttcttcctccctcacttctcacct

SEQ ID NO: 964 - DNA, 20 bp, Artificial Sequence
  Synthetic Oligonucleotide
cacccttggactggaaaact

SEQ ID NO: 965 - DNA, 20 bp, Artificial Sequence
  Synthetic Oligonucleotide
ccttgatgagctgttggttc

SEQ ID NO: 966 - DNA, 24 bp, Artificial Sequence
  Synthetic Oligonucleotide
cacacccggtctggacacagaaag

SEQ ID NO: 967 - DNA, 19 bp, Artificial Sequence
  Synthetic Oligonucleotide
gatcctcgcgtgagacaga

SEQ ID NO: 968 - DNA, 21 bp, Artificial Sequence
  Synthetic Oligonucleotide
cactttttctttgaccaaccg

SEQ ID NO: 969 - DNA, 23 bp, Artificial Sequence
  Synthetic Oligonucleotide
ttcaccacgccggtcttgatctt

SEQ ID NO: 970 - DNA, 20 bp, Artificial Sequence
  Synthetic Oligonucleotide
ctgttttcctggaggactgc

SEQ ID NO: 971 - DNA, 20 bp, Artificial Sequence
  Synthetic Oligonucleotide
actgtgtatgcggagctgtt

SEQ ID NO: 972 - DNA, 21 bp, Artificial Sequence
  Synthetic Oligonucleotide
ccactgccagcacgcagtcac

SEQ ID NO: 973 - DNA, 21 bp, Artificial Sequence
  Synthetic Oligonucleotide
cagccaggtgtacgagacatt

SEQ ID NO: 974 - DNA, 18 bp, Artificial Sequence
  Synthetic Oligonucleotide
acctcgtccagcacatcc

SEQ ID NO: 975 - DNA, 24 bp, Artificial Sequence
  Synthetic Oligonucleotide
ctcacctacagtgacgtcgtgggc

SEQ ID NO: 976 - DNA, 19 bp, Artificial Sequence
  Synthetic Oligonucleotide
tcccgagagaggaaagcat

SEQ ID NO: 977 - DNA, 18 bp, Artificial Sequence
  Synthetic Oligonucleotide
gtcgggtgcctgcattta

SEQ ID NO: 978 - DNA, 24 bp, Artificial Sequence
  Synthetic Oligonucleotide
atacacagtcccttcgtggctccc

SEQ ID NO: 979 - DNA, 22 bp, Artificial Sequence
  Synthetic Oligonucleotide
cctggttgaagcctgttaatgc

SEQ ID NO: 980 - DNA, 26 bp, Artificial Sequence
  Synthetic Oligonucleotide
tgcagacttctcatatttgctaaagg

SEQ ID NO: 981 - DNA, 23 bp, Artificial Sequence
  Synthetic Oligonucleotide
ccgctgggttttccacacgttga

SEQ ID NO: 982 - DNA, 22 bp, Artificial Sequence
  Synthetic Oligonucleotide
ccagtgtgtgtaacagggtcac

SEQ ID NO: 983 - DNA, 18 bp, Artificial Sequence
  Synthetic Oligonucleotide
tatagccttgggccaccc

SEQ ID NO: 984 - DNA, 24 bp, Artificial Sequence
  Synthetic Oligonucleotide
agaattcgacagccagatgctcca

SEQ ID NO: 985 - DNA, 20 bp, Artificial Sequence
  Synthetic Oligonucleotide
gccaactgctttcatttgtg

SEQ ID NO: 986 - DNA, 20 bp, Artificial Sequence
  Synthetic Oligonucleotide
actcaggcccatttccttta

SEQ ID NO: 987 - DNA, 28 bp, Artificial Sequence
  Synthetic Oligonucleotide
agggatctgaaccaatacagagcagaca

SEQ ID NO: 988 - DNA, 22 bp, Artificial Sequence
  Synthetic Oligonucleotide
catccgcaaagtgactgaagag

SEQ ID NO: 989 - DNA, 27 bp, Artificial Sequence
  Synthetic Oligonucleotide
gtactgaactccgttgtgatagcatag

SEQ ID NO: 990 - DNA, 22 bp, Artificial Sequence
  Synthetic Oligonucleotide
ccaatgagctgaggcggcctcc

SEQ ID NO: 991 - DNA, 20 bp, Artificial Sequence
  Synthetic Oligonucleotide
gccgggaagaccgtaattgt

SEQ ID NO: 992 - DNA, 18 bp, Artificial Sequence
  Synthetic Oligonucleotide
cagcggcaccaggttcag

SEQ ID NO: 993 - DNA, 26 bp, Artificial Sequence
  Synthetic Oligonucleotide
caaatggcttcctctggaaggtccca

SEQ ID NO: 994 - DNA, 20 bp, Artificial Sequence
  Synthetic Oligonucleotide
aatccaagggggagagtgat

SEQ ID NO: 995 - DNA, 20 bp, Artificial Sequence
  Synthetic Oligonucleotide
gtacagattttgcccgagga

SEQ ID NO: 996 - DNA, 26 bp, Artificial Sequence
  Synthetic Oligonucleotide
catatggactttgactcagctgtggc

SEQ ID NO: 997 - DNA, 18 bp, Artificial Sequence
  Synthetic Oligonucleotide
gtgatgccttccctgtgg

SEQ ID NO: 998 - DNA, 19 bp, Artificial Sequence
  Synthetic Oligonucleotide
tcaggtagtcgggtgggtt

SEQ ID NO: 999 - DNA, 24 bp, Artificial Sequence
  Synthetic Oligonucleotide
tgcttctccagcatcttcacctcg

SEQ ID NO: 1000 - DNA, 24 bp, Artificial Sequence
  Synthetic Oligonucleotide
ctatatgcagccagagatgtgaca

SEQ ID NO: 1001 - DNA, 24 bp, Artificial Sequence
  Synthetic Oligonucleotide
ccacgagtttcttactgagaatgg

SEQ ID NO: 1002 - DNA, 23 bp, Artificial Sequence
  Synthetic Oligonucleotide
acagcctgccactcatcacagcc

SEQ ID NO: 1003 - DNA, 20 bp, Artificial Sequence
  Synthetic Oligonucleotide
tgctgttgctgagtctgttg

SEQ ID NO: 1004 - DNA, 20 bp, Artificial Sequence
  Synthetic Oligonucleotide
cttgcctggcttcacagata

SEQ ID NO: 1005 - DNA, 24 bp, Artificial Sequence
  Synthetic Oligonucleotide
ccagtccccagaagaccatgtctg

SEQ ID NO: 1006 - DNA, 22 bp, Artificial Sequence
  Synthetic Oligonucleotide
ggtgtcgatattgtcatgaacc

SEQ ID NO: 1007 - DNA, 27 bp, Artificial Sequence
  Synthetic Oligonucleotide
gtaatctttgatgtacttcttgtaggc

SEQ ID NO: 1008 - DNA, 27 bp, Artificial Sequence
  Synthetic Oligonucleotide
tcacctgcaggaaacaagtttcacaaa

SEQ ID NO: 1009 - DNA, 21 bp, Artificial Sequence
  Synthetic Oligonucleotide
gacgctggtctggtgaagatg

SEQ ID NO: 1010 - DNA, 20 bp, Artificial Sequence
  Synthetic Oligonucleotide
tgggtggttgttggacaatg

SEQ ID NO: 1011 - DNA, 23 bp, Artificial Sequence
  Synthetic Oligonucleotide
ccaggaaaccacgagcctccagc

SEQ ID NO: 1012 - DNA, 22 bp, Artificial Sequence
  Synthetic Oligonucleotide
cttcacagtgctcctgcagtct

SEQ ID NO: 1013 - DNA, 21 bp, Artificial Sequence
  Synthetic Oligonucleotide
catctgcttcagctcgttggt

SEQ ID NO: 1014 - DNA, 26 bp, Artificial Sequence
  Synthetic Oligonucleotide
aagtacacgtaagttacagccacaca

SEQ ID NO: 1015 - DNA, 18 bp, Artificial Sequence
  Synthetic Oligonucleotide
gcctcggtgtgcctttca

SEQ ID NO: 1016 - DNA, 19 bp, Artificial Sequence
  Synthetic Oligonucleotide
cgtgatgtgcgcaatcatg

SEQ ID NO: 1017 - DNA, 22 bp, Artificial Sequence
  Synthetic Oligonucleotide
catcgccagctacgccctgctc

SEQ ID NO: 1018 - DNA, 19 bp, Artificial Sequence
  Synthetic Oligonucleotide
ctggtcagccttcagggac

SEQ ID NO: 1019 - DNA, 18 bp, Artificial Sequence
  Synthetic Oligonucleotide
cttcagttctgggctggc

SEQ ID NO: 1020 - DNA, 23 bp, Artificial Sequence
  Synthetic Oligonucleotide
ctgagcaccgcctggtctctttc

SEQ ID NO: 1021 - DNA, 23 bp, Artificial Sequence
  Synthetic Oligonucleotide
tgcttgtcagttgtcaacacctt

SEQ ID NO: 1022 - DNA, 23 bp, Artificial Sequence
  Synthetic Oligonucleotide
tggagtggcaagtatttgatgct

SEQ ID NO: 1023 - DNA, 23 bp, Artificial Sequence
  Synthetic Oligonucleotide
tggccaacctgcctgtttccagc

SEQ ID NO: 1024 - DNA, 19 bp, Artificial Sequence
  Synthetic Oligonucleotide
tgtcaccccgactcaacgt

SEQ ID NO: 1025 - DNA, 22 bp, Artificial Sequence
  Synthetic Oligonucleotide
acgtggactgagatgcattcac

SEQ ID NO: 1026 - DNA, 21 bp, Artificial Sequence
  Synthetic Oligonucleotide
agacgcaccgcccggactcac

SEQ ID NO: 1027 - DNA, 20 bp, Artificial Sequence
  Synthetic Oligonucleotide
agctcaacatgcgtgagtgt

SEQ ID NO: 1028 - DNA, 19 bp, Artificial Sequence
  Synthetic Oligonucleotide
attgccgatctggactcct

SEQ ID NO: 1029 - DNA, 23 bp, Artificial Sequence
  Synthetic Oligonucleotide
atctctatccacgtggggcaggc

SEQ ID NO: 1030 - DNA, 23 bp, Artificial Sequence
  Synthetic Oligonucleotide
ctcttacatcgaccgcctaagag

SEQ ID NO: 1031 - DNA, 18 bp, Artificial Sequence
  Synthetic Oligonucleotide
gctgatggcggagacgaa

SEQ ID NO: 1032 - DNA, 27 bp, Artificial Sequence
  Synthetic Oligonucleotide
cgcgctgtaagaagcaacaacctctcc

SEQ ID NO: 1033 - DNA, 20 bp, Artificial Sequence
  Synthetic Oligonucleotide
gaggagggtgagttctccaa

SEQ ID NO: 1034 - DNA, 20 bp, Artificial Sequence
  Synthetic Oligonucleotide
atgcccacctccttgtaatc

SEQ ID NO: 1035 - DNA, 24 bp, Artificial Sequence
  Synthetic Oligonucleotide
ccatgaggatatgactgccctgga

SEQ ID NO: 1036 - DNA, 19 bp, Artificial Sequence
  Synthetic Oligonucleotide
gtcccttcgcctccttcac

SEQ ID NO: 1037 - DNA, 20 bp, Artificial Sequence
  Synthetic Oligonucleotide
cgtggatggagatgcactca

SEQ ID NO: 1038 - DNA, 28 bp, Artificial Sequence
  Synthetic Oligonucleotide
ccgcagaccccttcaagttctagtcatg

SEQ ID NO: 1039 - DNA, 20 bp, Artificial Sequence
  Synthetic Oligonucleotide
cgccctacctataccaacct

SEQ ID NO: 1040 - DNA, 20 bp, Artificial Sequence
  Synthetic Oligonucleotide
cggagagaagcagtgattga

SEQ ID NO: 1041 - DNA, 25 bp, Artificial Sequence
  Synthetic Oligonucleotide
caaccgcctcatcagtcagattgtg

SEQ ID NO: 1042 - DNA, 20 bp, Artificial Sequence
  Synthetic Oligonucleotide
cgaggacgaggcttaaaaac

SEQ ID NO: 1043 - DNA, 20 bp, Artificial Sequence
  Synthetic Oligonucleotide
accatgcttgaggacaacag

SEQ ID NO: 1044 - DNA, 29 bp, Artificial Sequence
  Synthetic Oligonucleotide
tctcagatcaatcgtgcatccttagtgaa

SEQ ID NO: 1045 - DNA, 21 bp, Artificial Sequence
  Synthetic Oligonucleotide
cgccctcctgcagtatttatg

SEQ ID NO: 1046 - DNA, 22 bp, Artificial Sequence
  Synthetic Oligonucleotide
acagagacaggagcagctcaca

SEQ ID NO: 1047 - DNA, 23 bp, Artificial Sequence
  Synthetic Oligonucleotide
cctcgtcctccccacctaggcca

SEQ ID NO: 1048 - DNA, 21 bp, Artificial Sequence
  Synthetic Oligonucleotide
acactgactggcatcctgctt

SEQ ID NO: 1049 - DNA, 25 bp, Artificial Sequence
  Synthetic Oligonucleotide
gctctgtagctccccatgtactagt

SEQ ID NO: 1050 - DNA, 24 bp, Artificial Sequence
  Synthetic Oligonucleotide
agcctccagaagagccaggtgcct

SEQ ID NO: 1051 - DNA, 20 bp, Artificial Sequence
  Synthetic Oligonucleotide
gtggcctagagccttcagtc

SEQ ID NO: 1052 - DNA, 21 bp, Artificial Sequence
  Synthetic Oligonucleotide
caggctgggagtgaataaaga

SEQ ID NO: 1053 - DNA, 24 bp, Artificial Sequence
  Synthetic Oligonucleotide
ttcacactgcttccctgctttccc

SEQ ID NO: 1054 - DNA, 18 bp, Artificial Sequence
  Synthetic Oligonucleotide
acaggccccatgcatcct

SEQ ID NO: 1055 - DNA, 25 bp, Artificial Sequence
  Synthetic Oligonucleotide
tgtttctctcccagataagctaagg

SEQ ID NO: 1056 - DNA, 21 bp, Artificial Sequence
  Synthetic Oligonucleotide
tgcctcactcccctcagcccc

SEQ ID NO: 1057 - DNA, 19 bp, Artificial Sequence
  Synthetic Oligonucleotide
ccctatggccctgaatggt

SEQ ID NO: 1058 - DNA, 26 bp, Artificial Sequence
  Synthetic Oligonucleotide
actaattacatgacttggctgcattt

SEQ ID NO: 1059 - DNA, 23 bp, Artificial Sequence
  Synthetic Oligonucleotide
tgaggggccgacaccaacacaat

SEQ ID NO: 1060 - DNA, 20 bp, Artificial Sequence
  Synthetic Oligonucleotide
agtggaatccttccctttcc

SEQ ID NO: 1061 - DNA, 20 bp, Artificial Sequence
  Synthetic Oligonucleotide
cccttgatccctttctctga

SEQ ID NO: 1062 - DNA, 24 bp, Artificial Sequence
  Synthetic Oligonucleotide
cctcactcagctcctttcccctga

SEQ ID NO: 1063 - DNA, 24 bp, Artificial Sequence
  Synthetic Oligonucleotide
ggaaggaaagaagcatggtctact

SEQ ID NO: 1064 - DNA, 24 bp, Artificial Sequence
  Synthetic Oligonucleotide
aaaaagtgacaggcaacagtgaag

SEQ ID NO: 1065 - DNA, 24 bp, Artificial Sequence
  Synthetic Oligonucleotide
caccagagacccagcgcacaccta

SEQ ID NO: 1066 - DNA, 19 bp, Artificial Sequence
  Synthetic Oligonucleotide
gatgccgagggaaatcatc

SEQ ID NO: 1067 - DNA, 20 bp, Artificial Sequence
  Synthetic Oligonucleotide
ccagaactcgaacccaatct

SEQ ID NO: 1068 - DNA, 24 bp, Artificial Sequence
  Synthetic Oligonucleotide
attgccgcactggcccaactgtag

SEQ ID NO: 1069 - DNA, 18 bp, Artificial Sequence
  Synthetic Oligonucleotide
gcgctgcggaagatcatc

SEQ ID NO: 1070 - DNA, 22 bp, Artificial Sequence
  Synthetic Oligonucleotide
gcttgagggtctgaatcttgct

SEQ ID NO: 1071 - DNA, 21 bp, Artificial Sequence
  Synthetic Oligonucleotide
ccacgctgccctcggacaagc

SEQ ID NO: 1072 - DNA, 18 bp, Artificial Sequence
  Synthetic Oligonucleotide
cagtgtggaggggatgga

SEQ ID NO: 1073 - DNA, 19 bp, Artificial Sequence
  Synthetic Oligonucleotide
caagttctggaccacagcc

SEQ ID NO: 1074 - DNA, 24 bp, Artificial Sequence
  Synthetic Oligonucleotide
cttcacccactggatgtcaggctc

SEQ ID NO: 1075 - DNA, 23 bp, Artificial Sequence
  Synthetic Oligonucleotide
agttgtcgtgtgttctggattca

SEQ ID NO: 1076 - DNA, 22 bp, Artificial Sequence
  Synthetic Oligonucleotide
cgtcagcgtgatcttaaaggaa

SEQ ID NO: 1077 - DNA, 22 bp, Artificial Sequence
  Synthetic Oligonucleotide
tccggcaccaccatgtcgaagg

SEQ ID NO: 1078 - DNA, 19 bp, Artificial Sequence
  Synthetic Oligonucleotide
gtggatgtgccctgaagga

SEQ ID NO: 1079 - DNA, 20 bp, Artificial Sequence
  Synthetic Oligonucleotide
ctgcggatccagggtaagaa

SEQ ID NO: 1080 - DNA, 28 bp, Artificial Sequence
  Synthetic Oligonucleotide
aagccaggcgtctacacgagagtctcac

SEQ ID NO: 1081 - DNA, 24 bp, Artificial Sequence
  Synthetic Oligonucleotide
ggttgtgctctacgagcttatgac

SEQ ID NO: 1082 - DNA, 22 bp, Artificial Sequence
  Synthetic Oligonucleotide
cggcccaccataaagataatct

SEQ ID NO: 1083 - DNA, 24 bp, Artificial Sequence
  Synthetic Oligonucleotide
tgccttacagccacattggctgcc

SEQ ID NO: 1084 - DNA, 21 bp, Artificial Sequence
  Synthetic Oligonucleotide
tggcttcaggagctgaatacc

SEQ ID NO: 1085 - DNA, 24 bp, Artificial Sequence
  Synthetic Oligonucleotide
tgctgtcgtgatgagaaaatagtg

SEQ ID NO: 1086 - DNA, 26 bp, Artificial Sequence
  Synthetic Oligonucleotide
caggcacacacaggtgggacacaaat

SEQ ID NO: 1087 - DNA, 20 bp, Artificial Sequence
  Synthetic Oligonucleotide
ctgctgtcttgggtgcattg

SEQ ID NO: 1088 - DNA, 18 bp, Artificial Sequence
  Synthetic Oligonucleotide
gcagcctgggaccacttg

SEQ ID NO: 1089 - DNA, 25 bp, Artificial Sequence
  Synthetic Oligonucleotide
ttgccttgctgctctacctccacca

SEQ ID NO: 1090 - DNA, 19 bp, Artificial Sequence
  Synthetic Oligonucleotide
tgacgatggcctggagtgt

SEQ ID NO: 1091 - DNA, 22 bp, Artificial Sequence
  Synthetic Oligonucleotide
ggtaccggatcatgaggatctg

SEQ ID NO: 1092 - DNA, 21 bp, Artificial Sequence
  Synthetic Oligonucleotide
ctgggcagcaccaagtccgga

SEQ ID NO: 1093 - DNA, 25 bp, Artificial Sequence
  Synthetic Oligonucleotide
cctcagcaagacgttatttgaaatt

SEQ ID NO: 1094 - DNA, 24 bp, Artificial Sequence
  Synthetic Oligonucleotide
aagtgtgattggcaaaactgattg

SEQ ID NO: 1095 - DNA, 26 bp, Artificial Sequence
  Synthetic Oligonucleotide
cctctctctcaaggccccaaaccagt

SEQ ID NO: 1096 - DNA, 19 bp, Artificial Sequence
  Synthetic Oligonucleotide
cggttggtgacttgtctgc

SEQ ID NO: 1097 - DNA, 20 bp, Artificial Sequence
  Synthetic Oligonucleotide
aagacttgtccctgcctcac

SEQ ID NO: 1098 - DNA, 24 bp, Artificial Sequence
  Synthetic Oligonucleotide
atgcctcagtcttcccaaagcagg

SEQ ID NO: 1099 - DNA, 21 bp, Artificial Sequence
  Synthetic Oligonucleotide
tgcccttaaaggaaccaatga

SEQ ID NO: 1100 - DNA, 22 bp, Artificial Sequence
  Synthetic Oligonucleotide
gcttcaacggcaaagttctctt

SEQ ID NO: 1101 - DNA, 22 bp, Artificial Sequence
  Synthetic Oligonucleotide
atttcacgcatctggcgttcca

SEQ ID NO: 1102 - DNA, 18 bp, Artificial Sequence
  Synthetic Oligonucleotide
ctctccagtgtgggcacc

SEQ ID NO: 1103 - DNA, 19 bp, Artificial Sequence
  Synthetic Oligonucleotide
gcggtgtagctcccagagt

SEQ ID NO: 1104 - DNA, 24 bp, Artificial Sequence
  Synthetic Oligonucleotide
ccagaacagatgcgagcagtccat

SEQ ID NO: 1105 - DNA, 25 bp, Artificial Sequence
  Synthetic Oligonucleotide
gtatcaggaccacatgcagtacatc

SEQ ID NO: 1106 - DNA, 22 bp, Artificial Sequence
  Synthetic Oligonucleotide
tgtcggaattgatactggcatt

SEQ ID NO: 1107 - DNA, 24 bp, Artificial Sequence
  Synthetic Oligonucleotide
ttgatgcctgtcttcgcgccttct

SEQ ID NO: 1108 - DNA, 23 bp, Artificial Sequence
  Synthetic Oligonucleotide
gcagttggaagacacaggaaagt

SEQ ID NO: 1109 - DNA, 21 bp, Artificial Sequence
  Synthetic Oligonucleotide
tgcgtggcactattttcaaga

SEQ ID NO: 1110 - DNA, 27 bp, Artificial Sequence
  Synthetic Oligonucleotide
tccccaaattgcagatttatcaacggc

SEQ ID NO: 1111 - DNA, 20 bp, Artificial Sequence
  Synthetic Oligonucleotide
caggtcaggcagaggaagtc

SEQ ID NO: 1112 - DNA, 20 bp, Artificial Sequence
  Synthetic Oligonucleotide
cctaacaagccccaaatcaa

SEQ ID NO: 1113 - DNA, 27 bp, Artificial Sequence
  Synthetic Oligonucleotide
tgcattgcatgagctaaacctatctga

SEQ ID NO: 1114 - DNA, 20 bp, Artificial Sequence
  Synthetic Oligonucleotide
tggtcagatgctgctgaagt

SEQ ID NO: 1115 - DNA, 20 bp, Artificial Sequence
  Synthetic Oligonucleotide
atcacagcatgaagggatgg

SEQ ID NO: 1116 - DNA, 24 bp, Artificial Sequence
  Synthetic Oligonucleotide
tatccttaggccgctggcatcttg

SEQ ID NO: 1117 - DNA, 19 bp, Artificial Sequence
  Synthetic Oligonucleotide
gagggagcagacagtgggt

SEQ ID NO: 1118 - DNA, 21 bp, Artificial Sequence
  Synthetic Oligonucleotide
tcagagcccttgctaagtcac

SEQ ID NO: 1119 - DNA, 24 bp, Artificial Sequence
  Synthetic Oligonucleotide
ccacgatctccgtaaccatttgca

SEQ ID NO: 1120 - DNA, 20 bp, Artificial Sequence
  Synthetic Oligonucleotide
tagaggccatcaaaattggc

SEQ ID NO: 1121 - DNA, 18 bp, Artificial Sequence
  Synthetic Oligonucleotide
tccgtttcctctgggctt

SEQ ID NO: 1122 - DNA, 24 bp, Artificial Sequence
  Synthetic Oligonucleotide
accaaggccctgactcagatggag

SEQ ID NO: 1123 - DNA, 82 bp, Artificial Sequence
  Synthetic Oligonucleotide
ttacccgtgggaactgtctccaaatacatacttcctctcaccaggacaacaaccacaggatcctctgacccatttactggtc

SEQ ID NO: 1124 - DNA, 77 bp, Artificial Sequence
  Synthetic Oligonucleotide
aaacaccactggagcattgactaccaggctcgccaatgatgctgctcaagttaaaggggctataggttccaggcttg

SEQ ID NO: 1125 - DNA, 67 bp, Artificial Sequence
  Synthetic Oligonucleotide
agacagtcgccttggtcggtctcaatggcagtgggaagagtacggtagtccagcttctgcagaggtt

SEQ ID NO: 1126 - DNA, 68 bp, Artificial Sequence
  Synthetic Oligonucleotide
accagtgccacaatgcagtggctggacattcggctacagctcatgggggcggcagtggtcagcgctat

SEQ ID NO: 1127 - DNA, 66 bp, Artificial Sequence
  Synthetic Oligonucleotide
aagccacagcctccattgacatggagacagacaccctgatccagcgcacaatccgtgaagccttcc

SEQ ID NO: 1128 - DNA, 76 bp, Artificial Sequence
  Synthetic Oligonucleotide
tgcagactgtaccatgctgaccattgcccatcgcctgcacacggttctaggctccgataggattatggtgctggcc

SEQ ID NO: 1129 - DNA, 67 bp, Artificial Sequence
  Synthetic Oligonucleotide
tctgtggcccacctctactccaacctgaccaagccactcctggacgtggctgtgacttcctacaccc

SEQ ID NO: 1130 - DNA, 83 bp, Artificial Sequence
  Synthetic Oligonucleotide
atgtacgtcgccattcaagctgtgctctccctctatgcctctggccgcacgacaggcatcgtcctggattcaggtgatggcgt

SEQ ID NO: 1131 - DNA, 66 bp, Artificial Sequence
  Synthetic Oligonucleotide
atccgcattgaagacccaccccgcagaaagcacatggtattcctgggtggtgcagttctagcggat

SEQ ID NO: 1132 - DNA, 74 bp, Artificial Sequence
  Synthetic Oligonucleotide
caactgctgagagaccgagaagtaggaatccctccagaacaatccttggaaactgctaaggcagtaaaggagcg

SEQ ID NO: 1133 - DNA, 78 bp, Artificial Sequence
  Synthetic Oligonucleotide
ctgcatgtgattgaataagaaacaagaaagtgaccacaccaaagcctccctggctggtgtacagggatcaggtccaca

SEQ ID NO: 1134 - DNA, 71 bp, Artificial Sequence
  Synthetic Oligonucleotide
cgcttctatggcgctgagattgtgtcagccctggactacctgcactcggagaagaacgtggtgtaccggga

SEQ ID NO: 1135 - DNA, 71 bp, Artificial Sequence
  Synthetic Oligonucleotide
tcctgccacccttcaaacctcaggtcacgtccgaggtcgacacaaggtacttcgatgatgaatttaccgcc

SEQ ID NO: 1136 - DNA, 75 bp, Artificial Sequence
  Synthetic Oligonucleotide
ttgtctctgccttggactatctacattccggaaagattgtgtaccgtgatctcaagttggagaatctaatgctgg

SEQ ID NO: 1137 - DNA, 67 bp, Artificial Sequence
  Synthetic Oligonucleotide
tgggagggatgaaggaaattatctggacgatgctctcgtgagacaggatgcccaggacctgtatgag

SEQ ID NO: 1138 - DNA, 69 bp, Artificial Sequence
  Synthetic Oligonucleotide
ggacagcaggaatgtgtttctccatacaggtcacggggagccaatggttcagaaacaaatcgagtgggt

SEQ ID NO: 1139 - DNA, 68 bp, Artificial Sequence
  Synthetic Oligonucleotide
gatgaagcctttcgcaagttcctgaagggcctggcttcccgaaagccccttgtgctgtgtggagacct

SEQ ID NO: 1140 - DNA, 74 bp, Artificial Sequence
  Synthetic Oligonucleotide
ggaaacacactggaggacaaggctcgggaactcatcagccgcatcaaacagagtgaactttctgccaagatgcg

SEQ ID NO: 1141 - DNA, 67 bp, Artificial Sequence
  Synthetic Oligonucleotide
gtttatgccatcggcaccgtactggatcctggccaccgactatgagaactatgccctcgtgtattcc

SEQ ID NO: 1142 - DNA, 75 bp, Artificial Sequence
  Synthetic Oligonucleotide
gcctcaagagctggttcgagcccctggtggaagacatgcagcgccagtgggccgggctggtggagaaggtgcagg

SEQ ID NO: 1143 - DNA, 66 bp, Artificial Sequence
  Synthetic Oligonucleotide
gaggtcctggagtgcgtgagcctggtggagctgacctcgcttaagggcagggagaagctggcacct

SEQ ID NO: 1144 - DNA, 73 bp, Artificial Sequence
  Synthetic Oligonucleotide
ggtcctccgtcggttctctcattagtccacggtctggtcttcagctacccgccttcgtctccgagtttgcgac

SEQ ID NO: 1145 - DNA, 67 bp, Artificial Sequence
  Synthetic Oligonucleotide
agctgcagaagagctgcacatttgacgagcagcgaacagccacgatcatggaggagttggcagatgc

SEQ ID NO: 1146 - DNA, 66 bp, Artificial Sequence
  Synthetic Oligonucleotide
cagcagatgtggatcagcaagcaggagtatgacgagtccggcccctccatcgtccaccgcaaatgc

SEQ ID NO: 1147 - DNA, 73 bp, Artificial Sequence
  Synthetic Oligonucleotide
gggtcaggtgcctcgagatcgggcttgggcccagagcatgttccagatcccagagtttgagccgagtgagcag

SEQ ID NO: 1148 - DNA, 81 bp, Artificial Sequence
  Synthetic Oligonucleotide
cgttgtcagcacttggaatacaagatggttgccgggtcatgttaattgggaaaaagaacagtccacaggaagaggttgaac

SEQ ID NO: 1149 - DNA, 66 bp, Artificial Sequence
  Synthetic Oligonucleotide
ccattcccaccattctacctgaggccaggacgtctggggtgtggggattggtgggtctatgttccc

SEQ ID NO: 1150 - DNA, 70 bp, Artificial Sequence
  Synthetic Oligonucleotide
ccgccgtggacacagactccccccgagaggtctttttccgagtggcagctgacatgttttctgacggcaa

SEQ ID NO: 1151 - DNA, 83 bp, Artificial Sequence
  Synthetic Oligonucleotide
cctggagggtcctgtacaatctcatcatgggactcctgcccttacccaggggccacagagcccccgagatggagcccaattag

SEQ ID NO: 1152 - DNA, 80 bp, Artificial Sequence
  Synthetic Oligonucleotide
ggctcttgtgcgtactgtccttcgggctggtgacagggaagacatcactgagcctgccatctgtgctcttcgtcatctga

SEQ ID NO: 1153 - DNA, 73 bp, Artificial Sequence
  Synthetic Oligonucleotide
cagatggacctagtacccactgagatttccacgccgaaggacagcgatgggaaaaatgcccttaaatcatagg

SEQ ID NO: 1154 - DNA, 69 bp, Artificial Sequence
  Synthetic Oligonucleotide
aattaccaagcagccgaagaccacccacgaatggttatcttacgactgttacgttacattgtccgcctg

SEQ ID NO: 1155 - DNA, 70 bp, Artificial Sequence
  Synthetic Oligonucleotide
cagcgacaactctggacaagtcagtcccccagagtctccaactgtgaccacttcctggcagtctgagagc

SEQ ID NO: 1156 - DNA, 70 bp, Artificial Sequence
  Synthetic Oligonucleotide
cttttgtggaactctatgggaacaatgcagcagccgagagccgaaagggccaggaacgcttcaaccgctg

SEQ ID NO: 1157 - DNA, 74 bp, Artificial Sequence
  Synthetic Oligonucleotide
tgtactggcgaagaatatttggtaaagcagggcatcgatctctcaccctggggcttgtggaagaatcacgtggc

SEQ ID NO: 1158 - DNA, 71 bp, Artificial Sequence
  Synthetic Oligonucleotide
ggactgtgaggtcaacaacggttccagcctcagggatgagtgcatcacaaacctactggtgtttggcttcc

SEQ ID NO: 1159 - DNA, 76 bp, Artificial Sequence
  Synthetic Oligonucleotide
cctgcaaaagggaacaagagcccttcgcctccagatggctcccctgccgccacccccgagatcagagtcaaccacg

SEQ ID NO: 1160 - DNA, 65 bp, Artificial Sequence
  Synthetic Oligonucleotide
tcagggggctagaaatctgttgctatgggcccttcaccaacatgcccacagatcaactggaatgg

SEQ ID NO: 1161 - DNA, 70 bp, Artificial Sequence
  Synthetic Oligonucleotide
agttcgtgctttgcaagatggtgcagagctttatgaagcagtgaagaatgcagcagacccagcttacctt

SEQ ID NO: 1162 - DNA, 68 bp, Artificial Sequence
  Synthetic Oligonucleotide
ccgaggttaatccagcacgtatggggccaagtgtaggctcccagcaggaactgagagcgccatgtctt

SEQ ID NO: 1163 - DNA, 82 bp, Artificial Sequence
  Synthetic Oligonucleotide
tcaacagaaggctgaaccactagaaagactacagtcccagcaccgacaattccaagctcgagtgtctcggcaaactctgttg

SEQ ID NO: 1164 - DNA, 73 bp, Artificial Sequence
  Synthetic Oligonucleotide
ctgaagcagatggttcatcatttcctgggctgttaaacaaagcgaggttaaggttagactcttgggaatcagc

SEQ ID NO: 1165 - DNA, 69 bp, Artificial Sequence
  Synthetic Oligonucleotide
gtcagcgtggtagcggtattctccgcggcagtgacagtaattgtttttgcctctttagccaagacttcc

SEQ ID NO: 1166 - DNA, 65 bp, Artificial Sequence
  Synthetic Oligonucleotide
tcagctgtgagctgcggataccgcccggcaatgggacctgctcttaacctcaaacctaggaccgt

SEQ ID NO: 1167 - DNA, 72 bp, Artificial Sequence
  Synthetic Oligonucleotide
atcctagccctggtttttggcctcctttttgctgtcaccagcgtcgcgttccttgtgcagatgagaaggcag

SEQ ID NO: 1168 - DNA, 78 bp, Artificial Sequence
  Synthetic Oligonucleotide
cactaaggtttgagacagttccagaaagaacccaagctcaagacgcaggacgagctcagttgtagagggctaattcgc

SEQ ID NO: 1169 - DNA, 66 bp, Artificial Sequence
  Synthetic Oligonucleotide
tcgttggagatcagagtggaagttcaccatcacaccacctacagcccaggtggttggcgtgcttaa

SEQ ID NO: 1170 - DNA, 74 bp, Artificial Sequence
  Synthetic Oligonucleotide
gtggctcaacattgtgttcccatttcagctgatcagtgggcctccaaggaggggctgtaaaatggaggccattg

SEQ ID NO: 1171 - DNA, 84 bp, Artificial Sequence
  Synthetic Oligonucleotide
ttcaggttgttgcaggagaccatgtacatgactgtctccattattgatcggttcatgcagaataattgtgtgcccaagaagatg

SEQ ID NO: 1172 - DNA, 69 bp, Artificial Sequence
  Synthetic Oligonucleotide
gcatgttcgtggcctctaagatgaaggagaccatccccctgacggccgagaagctgtgcatctacaccg

SEQ ID NO: 1173 - DNA, 82 bp, Artificial Sequence
  Synthetic Oligonucleotide
atgctgtggctccttcctaactggggctttcttgacatgtaggttgcttggtaataacctttttgtatatcacaatttgggt

SEQ ID NO: 1174 - DNA, 69 bp, Artificial Sequence
  Synthetic Oligonucleotide
atccaaggccatgactggtgtggaacaatggccatacagggctgttgcccaggccctagaggtcattcc

SEQ ID NO: 1175 - DNA, 66 bp, Artificial Sequence
  Synthetic Oligonucleotide
gtgtgctagcgtactcccgcctcaaggaactgacgctcgaggacctaaagataaccggcaccatgc

SEQ ID NO: 1176 - DNA, 75 bp, Artificial Sequence
  Synthetic Oligonucleotide
tgtatttcaagacctctgtgcacttatttatgaacctgccctgctcccacagaacacagcaattcctcaggctaa

SEQ ID NO: 1177 - DNA, 65 bp, Artificial Sequence
  Synthetic Oligonucleotide
agatgaagtggaaggcgcttttcaccgcggccatcctgcaggcacagttgccgattacagaggca

SEQ ID NO: 1178 - DNA, 71 bp, Artificial Sequence
  Synthetic Oligonucleotide
agtgggactgattgccgtgggtgtcggggcacagcttgtcctgagtcagaccataatccagggggctaccc

SEQ ID NO: 1179 - DNA, 74 bp, Artificial Sequence
  Synthetic Oligonucleotide
tggttcccagccctgtgtccacctccaagcccagattcagattcgagtcatgtacacaacccagggtggaggag

SEQ ID NO: 1180 - DNA, 68 bp, Artificial Sequence
  Synthetic Oligonucleotide
gagagcgacgcggttgttgtagctgccgctgcggccgccgcggaataataagccgggatctaccatac

SEQ ID NO: 1181 - DNA, 68 bp, Artificial Sequence
  Synthetic Oligonucleotide
tggattggagttctgggaatgtactggccgtggcactggacaacagtgtgtacctgtggagtgcaagc

SEQ ID NO: 1182 - DNA, 85 bp, Artificial Sequence
  Synthetic Oligonucleotide
aaacgagcagtttgccatcagacgcttccagtctatgccggtgaggctgctgggccacagccccgtgcttcggaacatcaccaac

SEQ ID NO: 1183 - DNA, 67 bp, Artificial Sequence
  Synthetic Oligonucleotide
gaggcacagtattgcccagctggatccagaggccttgggaaacattaagaagctctccaaccgtctc

SEQ ID NO: 1184 - DNA, 81 bp, Artificial Sequence
  Synthetic Oligonucleotide
tgagtgtcccccggtatcttccccgccctgccaatcccgatgaaattggaaattttattgatgaaaatctgaaagcggctg

SEQ ID NO: 1185 - DNA, 67 bp, Artificial Sequence
  Synthetic Oligonucleotide
aagccctatccgatgtacccggccacaacatccctggtgaacgtcgtgcccaaactcaatgccacag

SEQ ID NO: 1186 - DNA, 69 bp, Artificial Sequence
  Synthetic Oligonucleotide
cggcgatcaagaagctgtccgggcctctgatctccgatttcttcgccaagcgcaagagatcagcgcctg

SEQ ID NO: 1187 - DNA, 77 bp, Artificial Sequence
  Synthetic Oligonucleotide
tgacaatcagcacacctgcattcaccgctcggaagagggcctgagctgcatgaataaggatcacggctgtagtcaca

SEQ ID NO: 1188 - DNA, 63 bp, Artificial Sequence
  Synthetic Oligonucleotide
taaattcactcgtggtgtggacttcaattggcaagcccaggccctattggccctacaagaggc

SEQ ID NO: 1189 - DNA, 83 bp, Artificial Sequence
  Synthetic Oligonucleotide
ggatgctggtgacctcttcttccctcacgttgcaacaggaattaaaggctaaaagaaaacgaagagttacttggtgccttggc

SEQ ID NO: 1190 - DNA, 68 bp, Artificial Sequence
  Synthetic Oligonucleotide
ctcccgtcaacagcgttctttccaaacactggaccaggagtgcatccagatgaaggccagactcaccc

SEQ ID NO: 1191 - DNA, 76 bp, Artificial Sequence
  Synthetic Oligonucleotide
ctgaaggagctccaagacctcgctctccaaggcgccaaggagagggcacatcagcagaagaaacacagcggttttg

SEQ ID NO: 1192 - DNA, 76 bp, Artificial Sequence
  Synthetic Oligonucleotide
aaggaagtggtccctctgtggcaagtgatgaagtctccagctttgcctcagctctcccagacagaaagactgcgtc

SEQ ID NO: 1193 - DNA, 82 bp, Artificial Sequence
  Synthetic Oligonucleotide
gataaattggtacaagggatcagcttttcccagcccacatgtcctgatcatatgcttttgaatagtcagttacttggcaccc

SEQ ID NO: 1194 - DNA, 78 bp, Artificial Sequence
  Synthetic Oligonucleotide
atgtggaacccccacctacttggcgcctgaagttcttgtttctgttgggactgctgggtataaccgtgctgtggactg

SEQ ID NO: 1195 - DNA, 86 bp, Artificial Sequence
  Synthetic Oligonucleotide
ggatatttccgtggctcttattcaaactctccatcaaatcctgtaaactccagagcaaatcaagatttttctgccttgatgagaag

SEQ ID NO: 1196 - DNA, 66 bp, Artificial Sequence
  Synthetic Oligonucleotide
tcattgaccacagtggcgcccgccttggtgagtatgaggacgtgtcccgggtggagaagtacacga

SEQ ID NO: 1197 - DNA, 76 bp, Artificial Sequence
  Synthetic Oligonucleotide
ccccaggatacctaccactacctgcccttcagcctgccccaccggaggcctcacttcttctttcccaagtcccgca

SEQ ID NO: 1198 - DNA, 86 bp, Artificial Sequence
  Synthetic Oligonucleotide
gacatttccagtcctgcagtcaatgcctctctgccccaccctttgttcagtgtggctggtgccacgacaaatgtgtgcgatcggag

SEQ ID NO: 1199 - DNA, 84 bp, Artificial Sequence
  Synthetic Oligonucleotide
tccctccactcggaaggactatcctgctgccaagagggtcaagttggacagtgtcagagtcctgagacagatcagcaacaaccg

SEQ ID NO: 1200 - DNA, 64 bp, Artificial Sequence
  Synthetic Oligonucleotide
tccaccctcctggctttggccagcatggcgaagacgaaaggaaacaaggtgaacgtgggagtga

SEQ ID NO: 1201 - DNA, 68 bp, Artificial Sequence
  Synthetic Oligonucleotide
gtggccatccagctgaccttcctgcgcctgatgtccaccgaggcctcccagaacatcacctaccactg

SEQ ID NO: 1202 - DNA, 80 bp, Artificial Sequence
  Synthetic Oligonucleotide
cagccaagaactggtataggagctccaaggacaagaaacacgtctggctaggagaaactatcaatgctggcagccagttt

SEQ ID NO: 1203 - DNA, 66 bp, Artificial Sequence
  Synthetic Oligonucleotide
gagagcaagcgagacattctgttcctctttgacggctcagccaatcttgtgggccagttccctgtt

SEQ ID NO: 1204 - DNA, 81 bp, Artificial Sequence
  Synthetic Oligonucleotide
cgacagttgcgatgaaagttctaatctcttccctcctcctgttgctgccactaatgctgatgtccatggtctctagcagcc

SEQ ID NO: 1205 - DNA, 79 bp, Artificial Sequence
  Synthetic Oligonucleotide
tctgcagagttggaagcactctatggtgacatcgatgctgtggagctgtatcctgcccttctggtagaaaagcctcggc

SEQ ID NO: 1206 - DNA, 78 bp, Artificial Sequence
  Synthetic Oligonucleotide
acctctgtggtccgtaggagccactatgaggagggccctgggaagaatttgccattttcagtggaaaacaagtggtcg

SEQ ID NO: 1207 - DNA, 67 bp, Artificial Sequence
  Synthetic Oligonucleotide
aacttcaaggtcggagaaggctttgaggaggagaccgtggacggacgcaagtgcaggagtttagcca

SEQ ID NO: 1208 - DNA, 76 bp, Artificial Sequence
  Synthetic Oligonucleotide
gtgctacgccaccctgttcggacccaaaggcgtgaacatcgggggcgcgggctcctacatctacgagaagcccctg

SEQ ID NO: 1209 - DNA, 69 bp, Artificial Sequence
  Synthetic Oligonucleotide
gatgtgattgaggtgcatggaaaacatgaagagcgccaggatgaacatggtttcatctccagggagttc

SEQ ID NO: 1210 - DNA, 74 bp, Artificial Sequence
  Synthetic Oligonucleotide
tgcagcggctgattgacagtcagatggagacctcgtgccaaattacatttgagtttgtagaccaggaacagttg

SEQ ID NO: 1211 - DNA, 72 bp, Artificial Sequence
  Synthetic Oligonucleotide
agcttttccggaatctgttccatcgccagggcttctcctatgactacgtgttcgactggaacatgctcaaat

SEQ ID NO: 1212 - DNA, 78 bp, Artificial Sequence
  Synthetic Oligonucleotide
tggcagaactacctgcaagaaaaaccagcacctgcgtctggatgactgtgacttccaaaccaaccacaccttgaagca

SEQ ID NO: 1213 - DNA, 80 bp, Artificial Sequence
  Synthetic Oligonucleotide
gtacatgatcccctgtgagaaggtgtccaccctgcccgcgatcacactgaagctgggaggcaaaggctacaagctgtccc

SEQ ID NO: 1214 - DNA, 74 bp, Artificial Sequence
  Synthetic Oligonucleotide
gggaggcttatctcactgagtgagcagaatctggtagactgctctgggcctcaaggcaatgaaggctgcaatgg

SEQ ID NO: 1215 - DNA, 67 bp, Artificial Sequence
  Synthetic Oligonucleotide
tgtctcactgagcgagcagaatctggtggactgttcgcgtcctcaaggcaatcagggctgcaatggt

SEQ ID NO: 1216 - DNA, 72 bp, Artificial Sequence
  Synthetic Oligonucleotide
tgaccgcttctaccccaatgacttgtgggtggttgtgttccagtttcagcacatcatggttggccttatcct

SEQ ID NO: 1217 - DNA, 77 bp, Artificial Sequence
  Synthetic Oligonucleotide
ggtgcctactccattgtggcgggcgtgtttgtgtgcctgctggagtacccccgggggaagaggaagaagggctccac

SEQ ID NO: 1218 - DNA, 71 bp, Artificial Sequence
  Synthetic Oligonucleotide
ccagctttgtgcctgtcactattcctcatgccaccactgccaacacctctgtcttgggctaccacattccc

SEQ ID NO: 1219 - DNA, 73 bp, Artificial Sequence
  Synthetic Oligonucleotide
ccgtgttcaagaggaagctcactgccttgtggaggagttgagaaaaaccaaggcttcaccctgtgatcccact

SEQ ID NO: 1220 - DNA, 79 bp, Artificial Sequence
  Synthetic Oligonucleotide
agaacaaggacaacatagatccttacatatacacaccctttggaagtggacccagaaactgcattggcatgaggtttgc

SEQ ID NO: 1221 - DNA, 70 bp, Artificial Sequence
  Synthetic Oligonucleotide
ccgtgtggctcgctttctgcagtgccgcttcctctttgcggggccctggttactcttcagcgaaatctcc

SEQ ID NO: 1222 - DNA, 73 bp, Artificial Sequence
  Synthetic Oligonucleotide
cacaatggcggctctgaagagttggctgtcgcgcagcgtaacttcattcttcaggtacagacagtgtttgtgt

SEQ ID NO: 1223 - DNA, 62 bp, Artificial Sequence
  Synthetic Oligonucleotide
caagcagtcaaggagaaccagaagcggcgggagacagaagaaaagatgaggcgagcaaaact

SEQ ID NO: 1224 - DNA, 68 bp, Artificial Sequence
  Synthetic Oligonucleotide
tccaattccagcatcactgtggagaaaagctgtttgtctccccagcatactttatcgccttcactgcc

SEQ ID NO: 1225 - DNA, 75 bp, Artificial Sequence
  Synthetic Oligonucleotide
cagtgcttccatggacaagtccttgtcaaaactggcccatgctgatggagatcaaacatcaatcatccctgtcca

SEQ ID NO: 1226 - DNA, 83 bp, Artificial Sequence
  Synthetic Oligonucleotide
tgcacagagggtgtgggttacaccaatgcttccaacaatttgtttgcttgcctcccatgtacagcttgtaaatcagatgaaga

SEQ ID NO: 1227 - DNA, 84 bp, Artificial Sequence
  Synthetic Oligonucleotide
ctctgagacagtgcttcgatgactttgcagacttggtgccctttgactcctgggagccgctcatgaggaagttgggcctcatgg

SEQ ID NO: 1228 - DNA, 76 bp, Artificial Sequence
  Synthetic Oligonucleotide
agacatcagctcctggttcaacgaggccattgacttcatagactccatcaagaatgctggaggaagggtgtttgtc

SEQ ID NO: 1229 - DNA, 74 bp, Artificial Sequence
  Synthetic Oligonucleotide
cagaggatgacgaggatgatgacattgacctgtttggcagtgacaatgaggaggaggacaaggaggcggcacag

SEQ ID NO: 1230 - DNA, 62 bp, Artificial Sequence
  Synthetic Oligonucleotide
tgtcgatggacttccagaaccacctgggcagctgccaaaagtgtgatccaagctgtcccaat

SEQ ID NO: 1231 - DNA, 82 bp, Artificial Sequence
  Synthetic Oligonucleotide
gatctaagatggcgactgtcgaaccggaaaccacccctactcctaatcccccgactacagaagaggagaaaacggaatctaa

SEQ ID NO: 1232 - DNA, 67 bp, Artificial Sequence
  Synthetic Oligonucleotide
aagccgcggttgaatgtgccatgaccctctccctctctggatggcaccatcattgaagctggcgtca

SEQ ID NO: 1233 - DNA, 67 bp, Artificial Sequence
  Synthetic Oligonucleotide
ctcggatcctagccctcgtgcctccatggactcgagtgtaccgagtacagagggatattccaatgcc

SEQ ID NO: 1234 - DNA, 76 bp, Artificial Sequence
  Synthetic Oligonucleotide
tggtccatcgccagttatcacatctgtatgcggaacctcaaaagagtccctggtgtgaagcaagatcgctagaaca

SEQ ID NO: 1235 - DNA, 81 bp, Artificial Sequence
  Synthetic Oligonucleotide
cggttatgtcatgccagatacacacctcaaaggtactccctcctcccgggaaggcaccctttcttcagtgggtctcagttc

SEQ ID NO: 1236 - DNA, 86 bp, Artificial Sequence
  Synthetic Oligonucleotide
tggctcttaatcagtttcgttacctgcctctggagaatttacgcattattcgtgggacaaaactttatgaggatcgatatgccttg

SEQ ID NO: 1237 - DNA, 67 bp, Artificial Sequence
  Synthetic Oligonucleotide
gtccaggtggatgtgaaagatccccagcaggccctcaaggagctggctaagatgtgtatcctggccg

SEQ ID NO: 1238 - DNA, 67 bp, Artificial Sequence
  Synthetic Oligonucleotide
acggatcacagtggaggaagcgctggctcacccctacctggagcagtactatgacccgacggatgag

SEQ ID NO: 1239 - DNA, 70 bp, Artificial Sequence
  Synthetic Oligonucleotide
acccccagaccggatcaggcaagctggccctcatgtccccttcacggtgtttgaggaagtctgccctaca

SEQ ID NO: 1240 - DNA, 68 bp, Artificial Sequence
  Synthetic Oligonucleotide
cgtggtgcccctctatgacctgctgctggagatgctggacgcccaccgcctacatgcgcccactagcc

SEQ ID NO: 1241 - DNA, 91 bp, Artificial Sequence
  Synthetic Oligonucleotide
ggattgctcaacaaccatgctgggcatctggaccctcctacctctggttcttacgtctgttgctagattatcgtccaaaagtgttaatgcc

SEQ ID NO: 1242 - DNA, 80 bp, Artificial Sequence
  Synthetic Oligonucleotide
gcactttgggattctttccattatgattctttgttacaggcaccgagaatgttgtattcagtgagggtcttcttacatgc

SEQ ID NO: 1243 - DNA, 66 bp, Artificial Sequence
  Synthetic Oligonucleotide
gcctcttcctgttcgacggctcgcccacctacgtactggcctacacccagagctaccgggcaaagc

SEQ ID NO: 1244 - DNA, 90 bp, Artificial Sequence
  Synthetic Oligonucleotide
ggctattcctcattttctctacaaagtggcctcagtgaacatgaagaaggtagcctcctggaggagaatttcggtgacagtctacaatcc

SEQ ID NO: 1245 - DNA, 67 bp, Artificial Sequence
  Synthetic Oligonucleotide
aaggaaagggtgcctcatcccagcaacctgtccttgtgggtgatgatcactgtgctgcttgtggctc

SEQ ID NO: 1246 - DNA, 74 bp, Artificial Sequence
  Synthetic Oligonucleotide
cacgggacattcaccacatcgactactataaaaagacaaccaacggccgactgcctgtgaagtggatggcaccc

SEQ ID NO: 1247 - DNA, 67 bp, Artificial Sequence
  Synthetic Oligonucleotide
ccagtggagcgcttccatgacctgcgtcctgatgaagtggccgatttgtttcagacgacccagagag

SEQ ID NO: 1248 - DNA, 72 bp, Artificial Sequence
  Synthetic Oligonucleotide
ggttccagctttctgtagctgtaagcattggtggccacaccacctccttacaaagcaactagaacctgcggc

SEQ ID NO: 1249 - DNA, 73 bp, Artificial Sequence
  Synthetic Oligonucleotide
gcgtatgatttcccgaatgagtcaaaatgttgatatgcccaaacttcatgatgcaatgggtacgaggtcactg

SEQ ID NO: 1250 - DNA, 67 bp, Artificial Sequence
  Synthetic Oligonucleotide
cgagccctttgatgacttcctgttcccagcatcatccaggcccagtggctctgagacagcccgctcc

SEQ ID NO: 1251 - DNA, 82 bp, Artificial Sequence
  Synthetic Oligonucleotide
ccaccccgagcaaatctgtcctccccagaacccctgaatcctggaggctcacgcccccagccaaagtagggggactggattt

SEQ ID NO: 1252 - DNA, 80 bp, Artificial Sequence
  Synthetic Oligonucleotide
ggataattcagacaacaacaccatctttgtgcaaggcctgggtgagaatgttacaattgagtctgtggctgattacttca

SEQ ID NO: 1253 - DNA, 69 bp, Artificial Sequence
  Synthetic Oligonucleotide
gaagcgcagatcatgaagaagctgaagcacgacaagctggtccagctctatgcagtggtgtctgaggag

SEQ ID NO: 1254 - DNA, 67 bp, Artificial Sequence
  Synthetic Oligonucleotide
cctccaactcctagcctcaagtgatcctcctgtctcaacctcccaagtaggattacaagcatgcgcc

SEQ ID NO: 1255 - DNA, 73 bp, Artificial Sequence
  Synthetic Oligonucleotide
gtgctggtgacgaatccacattcatctcaatggaaggatcctgccttaagtcaacttatttgtttttgccggg

SEQ ID NO: 1256 - DNA, 70 bp, Artificial Sequence
  Synthetic Oligonucleotide
accctcgacaagaccacactttgggacttgggagctggggctgaagttgctctgtacccatgaactccca

SEQ ID NO: 1257 - DNA, 84 bp, Artificial Sequence
  Synthetic Oligonucleotide
aagggcaatcacagtgttaaaagaagacatgctgaaatgttgcaggctgctcctatgttggaaaattcttcattgaagttctcc

SEQ ID NO: 1258 - DNA, 74 bp, Artificial Sequence
  Synthetic Oligonucleotide
attccacccatggcaaattccatggcaccgtcaaggctgagaacgggaagcttgtcatcaatggaaatcccatc

SEQ ID NO: 1259 - DNA, 75 bp, Artificial Sequence
  Synthetic Oligonucleotide
caaaggagctcactgtggtgtctgtgttccaaccactgaatctggaccccatctgtgaataagccattctgactc

SEQ ID NO: 1260 - DNA, 73 bp, Artificial Sequence
  Synthetic Oligonucleotide
ttgggaaatatttgggcattggtctggccaagtctacaatgtcccaatatcaaggacaaccaccctagcttct

SEQ ID NO: 1261 - DNA, 83 bp, Artificial Sequence
  Synthetic Oligonucleotide
gcatgggaaccatcaaccagcaggccatggaccaacttcactatgtgacagagctgacagatcgaatcaaggcaaactcctca

SEQ ID NO: 1262 - DNA, 71 bp, Artificial Sequence
  Synthetic Oligonucleotide
ctgttgcaggaaggcattgatcatctcctggcccagcatgttgctcatctctttattagagacccactgac

SEQ ID NO: 1263 - DNA, 72 bp, Artificial Sequence
  Synthetic Oligonucleotide
cgctccagacctatgatgacttgttagccaaagactgccactgcatatgagcagtcctggtccttccactgt

SEQ ID NO: 1264 - DNA, 70 bp, Artificial Sequence
  Synthetic Oligonucleotide
ctccgacgtggctttccagtggcccacagcatctatggaatcccatctgtcatcaattctgccaattacg

SEQ ID NO: 1265 - DNA, 67 bp, Artificial Sequence
  Synthetic Oligonucleotide
ggagctctgcagtaaccacagaacaggccccatgctgacgtccctcctcaagagctggatggcattg

SEQ ID NO: 1266 - DNA, 67 bp, Artificial Sequence
  Synthetic Oligonucleotide
ggtgaaggttgtctcttccgagggccttctgaagacagggctcttgaacagacaagtggaagggctg

SEQ ID NO: 1267 - DNA, 66 bp, Artificial Sequence
  Synthetic Oligonucleotide
tacccttccatgtgctggatccgggactccctggtcagctacatcaccaacctgggcctcttcagc

SEQ ID NO: 1268 - DNA, 67 bp, Artificial Sequence
  Synthetic Oligonucleotide
gcttatgaccgaccccaagctcatcacctggtctccggtgtgtcgcaacgatgttgcctggaacttt

SEQ ID NO: 1269 - DNA, 67 bp, Artificial Sequence
  Synthetic Oligonucleotide
ccatctgcatccatcttgtttgggctccccacccttgagaagtgcctcagataataccctggtggcc

SEQ ID NO: 1270 - DNA, 66 bp, Artificial Sequence
  Synthetic Oligonucleotide
gacaaggacggcagcaaggtgacaacagtggtggcaactcctgggcagggtccagacaggccacaa

SEQ ID NO: 1271 - DNA, 68 bp, Artificial Sequence
  Synthetic Oligonucleotide
gtgatcccaagcccacaatagaggtcagtgggaaaaagtacaccgccccacacatcctgatcgccaca

SEQ ID NO: 1272 - DNA, 86 bp, Artificial Sequence
  Synthetic Oligonucleotide
aagctatgaggaaaagaagtacacgatgggggacgctcctgattatgacagaagccagtggctgaatgaaaaattcaagctgggcc

SEQ ID NO: 1273 - DNA, 76 bp, Artificial Sequence
  Synthetic Oligonucleotide
gagaccctgctgtcccagaaccagggaggcaagaccttcattgtgggagaccagatctccttcgctgactacaacc

SEQ ID NO: 1274 - DNA, 73 bp, Artificial Sequence
  Synthetic Oligonucleotide
cccactcagtagccaagtcacaatgtttggaaaacagcccgtttacttgagcaagactgataccacctgcgtg

SEQ ID NO: 1275 - DNA, 66 bp, Artificial Sequence
  Synthetic Oligonucleotide
tcctgtgctctggaagcccttgagcccttctgggaggttcttgtgagatcaactgagaccgtggag

SEQ ID NO: 1276 - DNA, 70 bp, Artificial Sequence
  Synthetic Oligonucleotide
cggtgtgagaagtgcagcaagccctgtgcccgagtgtgctatggtctgggcatggagcacttgcgagagg

SEQ ID NO: 1277 - DNA, 82 bp, Artificial Sequence
  Synthetic Oligonucleotide
tgaacataaagtctgcaacatggaaggtattgcactgcacaggccacattcacgtatatgataccaacagtaaccaacctca

SEQ ID NO: 1278 - DNA, 73 bp, Artificial Sequence
  Synthetic Oligonucleotide
tccaggatgttaggaactgtgaagatggaagggcatgaaaccagcgactggaacagctactacgcagacacgc

SEQ ID NO: 1279 - DNA, 67 bp, Artificial Sequence
  Synthetic Oligonucleotide
ggacgaatacgaccccactatagaggattcctaccggaagcaggtggtcattgatggggagacgtgc

SEQ ID NO: 1280 - DNA, 70 bp, Artificial Sequence
  Synthetic Oligonucleotide
ctgctgcgacagtccactacctttttcgagagtgactcccgttgtcccaaggcttcccagagcgaacctg

SEQ ID NO: 1281 - DNA, 63 bp, Artificial Sequence
  Synthetic Oligonucleotide
ggtccgcttcgtctttcgagagtgactcccgcggtcccaaggctttccagagcgaacctgtgc

SEQ ID NO: 1282 - DNA, 72 bp, Artificial Sequence
  Synthetic Oligonucleotide
gcaggtgtgattgctggacttaatgtgctaagaatcatcaatgagcccacggctgctgccattgcctatggt

SEQ ID NO: 1283 - DNA, 84 bp, Artificial Sequence
  Synthetic Oligonucleotide
ggctagtagaactggatcccaacaccaaactcttaattagacctaggcctcagctgcactgcccgaaaagcatttgggcagacc

SEQ ID NO: 1284 - DNA, 70 bp, Artificial Sequence
  Synthetic Oligonucleotide
ggccactaaagatgctggccagatatctggactgaatgtgcttcgggtgattaatgagcccacagctgct

SEQ ID NO: 1285 - DNA, 84 bp, Artificial Sequence
  Synthetic Oligonucleotide
ccgactggaggagcataaaagcgcagccgagcccagcgccccgcacttttctgagcagacgtccagagcagagtcagccagcat

SEQ ID NO: 1286 - DNA, 71 bp, Artificial Sequence
  Synthetic Oligonucleotide
caaaaggcagaggctgataagaacgacaagtctgtgaaggatctggtcatcttgctttatgaaactgcgct

SEQ ID NO: 1287 - DNA, 70 bp, Artificial Sequence
  Synthetic Oligonucleotide
agaaccgcaaggtgagcaaggtggagattctccagcacgtcatcgactacatcagggaccttcagttgga

SEQ ID NO: 1288 - DNA, 79 bp, Artificial Sequence
  Synthetic Oligonucleotide
cacgcagaaaaccacacttctcaaaccttcactcaacacttccttccccaaagccagaagatgcacaaggaggaacatg

SEQ ID NO: 1289 - DNA, 83 bp, Artificial Sequence
  Synthetic Oligonucleotide
gcatggtagccgaagatttcacagtcaaaatcggagattttggtatgacgcgagatatctatgagacagactattaccggaaa

SEQ ID NO: 1290 - DNA, 73 bp, Artificial Sequence
  Synthetic Oligonucleotide
gtggacagcaccatgaacatgttgggcgggggaggcagtgctggccggaagcccctcaagtcgggtatgaagg

SEQ ID NO: 1291 - DNA, 68 bp, Artificial Sequence
  Synthetic Oligonucleotide
acgcaccgggtgtctgatcccaagttccaccccctccattcaaagataatcatcatcaagaaagggca

SEQ ID NO: 1292 - DNA, 69 bp, Artificial Sequence
  Synthetic Oligonucleotide
tggacaagtacgggatgaagctgccaggcatggagtacgttgacggggactttcagtgccacaccttcg

SEQ ID NO: 1293 - DNA, 74 bp, Artificial Sequence
  Synthetic Oligonucleotide
tctgcgtggttcctttctcagccgcttctgactgctgattctcccgttcacgttgcctaataaacatccttcaa

SEQ ID NO: 1294 - DNA, 72 bp, Artificial Sequence
  Synthetic Oligonucleotide
cctgaaccttccaaagatggctgaaaaagatggatgcttccaatctggattcaatgaggagacttgcctggt

SEQ ID NO: 1295 - DNA, 71 bp, Artificial Sequence
  Synthetic Oligonucleotide
gcggtgattcggaaattcgcgaattcctctggtcctcatccaggtgcgcgggaagcaggtgcccaggagag

SEQ ID NO: 1296 - DNA, 70 bp, Artificial Sequence
  Synthetic Oligonucleotide
aaggaaccatctcactgtgtgtaaacatgacttccaagctggccgtggctctcttggcagccttcctgat

SEQ ID NO: 1297 - DNA, 68 bp, Artificial Sequence
  Synthetic Oligonucleotide
ccgctccgtcactgatgtctacctgctgaacctagccttggccgacctactctttgccctgaccttgc

SEQ ID NO: 1298 - DNA, 66 bp, Artificial Sequence
  Synthetic Oligonucleotide
ctcaggattttctcgcatccaaatgtgctcccagtgctaggtgcctgccagtctccacctgctcct

SEQ ID NO: 1299 - DNA, 63 bp, Artificial Sequence
  Synthetic Oligonucleotide
agccatcactctcagtgcagccaggtcctatcgtggcccctgaggagaccctgactctgcagt

SEQ ID NO: 1300 - DNA, 67 bp, Artificial Sequence
  Synthetic Oligonucleotide
gccaggatactggagtccatcacagtgagctccctgatggctacaccccaggaccccaagggtcaag

SEQ ID NO: 1301 - DNA, 67 bp, Artificial Sequence
  Synthetic Oligonucleotide
ggatggagttcgcctcctacgtgatcacagacctgacccagctgcggaagatcaagtccatggagcg

SEQ ID NO: 1302 - DNA, 74 bp, Artificial Sequence
  Synthetic Oligonucleotide
ccacagctcaccttctgtcaggtgtccatcccagctccagccagctcccagagaggaagagactggcactgagg

SEQ ID NO: 1303 - DNA, 74 bp, Artificial Sequence
  Synthetic Oligonucleotide
tcagaattggatttggctcatttgtggaaaagactgtgatgccttacattagcacaacaccagctaagctcagg

SEQ ID NO: 1304 - DNA, 74 bp, Artificial Sequence
  Synthetic Oligonucleotide
tgaggaagaaggagaggaatactaattatccattccttttggccctgcagcatgtcatgctcccagaatttcag

SEQ ID NO: 1305 - DNA, 68 bp, Artificial Sequence
  Synthetic Oligonucleotide
gaggacgaaggcctctacacctgccaggcatgcagtgttcttggctgtgcaaaagtggaggcattttt

SEQ ID NO: 1306 - DNA, 80 bp, Artificial Sequence
  Synthetic Oligonucleotide
cggactttgggtgcgacttgacgagcggtggttcgacaagtggccttgcgggccggatcgtcccagtggaagagttgtaa

SEQ ID NO: 1307 - DNA, 71 bp, Artificial Sequence
  Synthetic Oligonucleotide
tggaggttgtaagccaatgttgtgaggcttcaagttcagacatcactgagaaatcagatggacgtaaggca

SEQ ID NO: 1308 - DNA, 67 bp, Artificial Sequence
  Synthetic Oligonucleotide
ctaaggcacttgctggaagggcagaatgccagtgtgcttgcctatggacccacaggagctgggaaga

SEQ ID NO: 1309 - DNA, 73 bp, Artificial Sequence
  Synthetic Oligonucleotide
aattcctgctccaaaagaaagtcttcgaagccgctccactcgcatgtccactgtctcagagcttcgcatcacg

SEQ ID NO: 1310 - DNA, 66 bp, Artificial Sequence
  Synthetic Oligonucleotide
ccacagggttgaagaaccagaagccagttcctgctgttcctgtccagaagtctggcacatcaggtg

SEQ ID NO: 1311 - DNA, 78 bp, Artificial Sequence
  Synthetic Oligonucleotide
gcccagaggctccatcgtccatcctcttcctccccagtcggctgaactctccccttgtctgcactgttcaaacctctg

SEQ ID NO: 1312 - DNA, 70 bp, Artificial Sequence
  Synthetic Oligonucleotide
caaacagagggtggcagaagtgctcaatgaccctgagaacatggagaagcgcaggagccgtgagagcctc

SEQ ID NO: 1313 - DNA, 72 bp, Artificial Sequence
  Synthetic Oligonucleotide
agccgaggctttgttgaagaagcttcatatccagtaccggcgatcgggcacagaagctgtgctcatagccca

SEQ ID NO: 1314 - DNA, 71 bp, Artificial Sequence
  Synthetic Oligonucleotide
atgtgccagtgagcttgagtccttggagaaacacaagcacctgctagaaagtactgttaaccaggggctca

SEQ ID NO: 1315 - DNA, 83 bp, Artificial Sequence
  Synthetic Oligonucleotide
ggcctgctgagatcaaagactacagtccctacttcaagaccattgaggacctgaggaacaagattctcacagccacagtggac

SEQ ID NO: 1316 - DNA, 73 bp, Artificial Sequence
  Synthetic Oligonucleotide
cgaggattggttcttcagcaagacagaggaactgaaccgcgaggtggccaccaacagtgagctggtgcagagt

SEQ ID NO: 1317 - DNA, 77 bp, Artificial Sequence
  Synthetic Oligonucleotide
tgagcggcagaatcaggagtaccagcggctcatggacatcaagtcgcggctggagcaggagattgccacctaccgca

SEQ ID NO: 1318 - DNA, 69 bp, Artificial Sequence
  Synthetic Oligonucleotide
tcagtggagaaggagttggaccagtcaacatctctgttgtcacaagcagtgtttcctctggatatggca

SEQ ID NO: 1319 - DNA, 66 bp, Artificial Sequence
  Synthetic Oligonucleotide
cttgctggccaatgcctacatctacgttgtccagctgccagccaagatcctgactgcggacaatca

SEQ ID NO: 1320 - DNA, 80 bp, Artificial Sequence
  Synthetic Oligonucleotide
actcaagcggaaattgaagcagataggtcttatcagcacagtctccgcctcctggattcagtgtctcggcttcagggagt

SEQ ID NO: 1321 - DNA, 67 bp, Artificial Sequence
  Synthetic Oligonucleotide
agcgatgaagatggtcgcgccctggacgcggttctactccaacagctgctgcttgtgctgccatgtc

SEQ ID NO: 1322 - DNA, 67 bp, Artificial Sequence
  Synthetic Oligonucleotide
gcttcaggtgttgtgactgcagtgcctccctgtcgcaccagtactatgagaaggatgggcagctctt

SEQ ID NO: 1323 - DNA, 66 bp, Artificial Sequence
  Synthetic Oligonucleotide
ctttgggccaggaggaatctgttactcgaatccacccaggaactccctggcagtggattgtgggag

SEQ ID NO: 1324 - DNA, 67 bp, Artificial Sequence
  Synthetic Oligonucleotide
agaagctgtccctgcaagagcaggatgcagcgattgtgaagaacatgaagtctgagctggtacggct

SEQ ID NO: 1325 - DNA, 75 bp, Artificial Sequence
  Synthetic Oligonucleotide
ccgggagcagggaatcaccctgcgcgggagcgccgaaatcgtggccgagttcttctcattcggcatcaacagcat

SEQ ID NO: 1326 - DNA, 76 bp, Artificial Sequence
  Synthetic Oligonucleotide
ctgtcatgtggcagaccttccatccgaaccacggcttgggaagactacatttggttccaggcaccagtgacattta

SEQ ID NO: 1327 - DNA, 66 bp, Artificial Sequence
  Synthetic Oligonucleotide
gcccagtggagaaattcgtctttgagatcacccagcctccactgctgtccatcagctcagactcgc

SEQ ID NO: 1328 - DNA, 72 bp, Artificial Sequence
  Synthetic Oligonucleotide
cctcagaaattgccaggacttctttcccgtgatcttcagcaaagcctccgagtacttgcagctggtctttgg

SEQ ID NO: 1329 - DNA, 74 bp, Artificial Sequence
  Synthetic Oligonucleotide
aggactccagcaaccaagaagaggaggggccaagcaccttccctgacctggagtctgagttccaagcagcactc

SEQ ID NO: 1330 - DNA, 67 bp, Artificial Sequence
  Synthetic Oligonucleotide
cggaccaccaggtcagagccaattcgcagagcagggaagagtggtacctcaacacccactacccctg

SEQ ID NO: 1331 - DNA, 67 bp, Artificial Sequence
  Synthetic Oligonucleotide
gccctccaatgtccttatcaacaaggagggccatgtgaagatgtgtgactttggcatcagtggctac

SEQ ID NO: 1332 - DNA, 72 bp, Artificial Sequence
  Synthetic Oligonucleotide
gccggtcaggcacacaaggggcccttggagcgtggactggttggttttgccattttgttgtgtgtatgctgc

SEQ ID NO: 1333 - DNA, 77 bp, Artificial Sequence
  Synthetic Oligonucleotide
ccctcaaccggcaaatccgcgaggaggtggcgagtgcagtgagcagctcctacaggaatgaattcagggcatggacg

SEQ ID NO: 1334 - DNA, 76 bp, Artificial Sequence
  Synthetic Oligonucleotide
tgagtggaaaagcctgacctatgatgaagtcatcagctttgtgccaccaccccttgaccaagaagagatggagtcc

SEQ ID NO: 1335 - DNA, 83 bp, Artificial Sequence
  Synthetic Oligonucleotide
caacacccgtacatcaatgtctggtatgatccttctgaagcagaagctccaccaccaaagatccctgacaagcagttagatga

SEQ ID NO: 1336 - DNA, 69 bp, Artificial Sequence
  Synthetic Oligonucleotide
gaccttggaacctttggaacctgctgtcaacaggtcttacagggctgcttgaaccctcataggcctagg

SEQ ID NO: 1337 - DNA, 67 bp, Artificial Sequence
  Synthetic Oligonucleotide
cacaagctgaccttccgcgagaacgccaaagccaagacagaccacggggcggagatcgtgtacaagt

SEQ ID NO: 1338 - DNA, 77 bp, Artificial Sequence
  Synthetic Oligonucleotide
cagatggccactttgagaacattttagctgacaacagtgtgaacgaccagaccaaaatccttgtggttaatgctgcc

SEQ ID NO: 1339 - DNA, 75 bp, Artificial Sequence
  Synthetic Oligonucleotide
cttcggaaactggacatcaaaaacgaagacgatgtgaaatcgttgtctcgagtgatgatccatgttttcagcgac

SEQ ID NO: 1340 - DNA, 75 bp, Artificial Sequence
  Synthetic Oligonucleotide
gacttttgcccgctacctttcattccggcgtgacaacaatgagctgttgctcttcatactgaagcagttagtggc

SEQ ID NO: 1341 - DNA, 82 bp, Artificial Sequence
  Synthetic Oligonucleotide
tgatggtcctatgtgtcacattcatcacaggtttcataccaacacaggcttcagcacttcctttggtgtgtttcctgtccca

SEQ ID NO: 1342 - DNA, 71 bp, Artificial Sequence
  Synthetic Oligonucleotide
cgctcagccagatgcaatcaatgccccagtcacctgctgttataacttcaccaataggaagatctcagtgc

SEQ ID NO: 1343 - DNA, 69 bp, Artificial Sequence
  Synthetic Oligonucleotide
gtgaaatgaaacgcaccacactggacagccctttggggaagctggagctgtctggttgtgagcagggtc

SEQ ID NO: 1344 - DNA, 78 bp, Artificial Sequence
  Synthetic Oligonucleotide
ccaacgcttgccaaatcctgacaattcagaaccagctctctgtgaccccaatttgagttttgatgctgtcactaccgt

SEQ ID NO: 1345 - DNA, 86 bp, Artificial Sequence
  Synthetic Oligonucleotide
ccatgatggagaggcagacatcatgatcaactttggccgctgggagcatggcgatggatacccctttgacggtaaggacggactcc

SEQ ID NO: 1346 - DNA, 67 bp, Artificial Sequence
  Synthetic Oligonucleotide
gagaaccaatctcaccgacaggcagctggcagaggaatacctgtaccgctatggttacactcgggtg

SEQ ID NO: 1347 - DNA, 78 bp, Artificial Sequence
  Synthetic Oligonucleotide
gccatgctggctcagtctgagctgtgggccacatcagctagtggctcttctcatgcatcagttaggtgggtctgggtg

SEQ ID NO: 1348 - DNA, 79 bp, Artificial Sequence
  Synthetic Oligonucleotide
tcatggtgcccgtcaatgctgtgatggcgatgaagaccaagacgtatcaggtggcccacatgaagagcaaagacaatcg

SEQ ID NO: 1349 - DNA, 65 bp, Artificial Sequence
  Synthetic Oligonucleotide
aggggatgacttggacacatctgccattcgacatgactgcaattttgacaaagccatgcagtttt

SEQ ID NO: 1350 - DNA, 91 bp, Artificial Sequence
  Synthetic Oligonucleotide
tcatcctggcgatctacttcctctggcagaacctaggtccctctgtcctggctggagtcgctttcatggtcttgctgattccactcaacgg

SEQ ID NO: 1351 - DNA, 82 bp, Artificial Sequence
  Synthetic Oligonucleotide
tgattaccatcatggctcagattggctcctatgttcctgcagaagaagcgacaattgggattgtggatggcattttcacaag

SEQ ID NO: 1352 - DNA, 71 bp, Artificial Sequence
  Synthetic Oligonucleotide
ggccaggatctgtggtggtacaattgactctggccttccgagaaggtaccatcaatgtccacgacgtggag

SEQ ID NO: 1353 - DNA, 78 bp, Artificial Sequence
  Synthetic Oligonucleotide
gaaggaatgggaatcagtcatgagctaatcaccctggagatcagctcccgagatgtcccggatctgactctaatagac

SEQ ID NO: 1354 - DNA, 74 bp, Artificial Sequence
  Synthetic Oligonucleotide
gccgagatcgccaagatgttgccagggaggacagacaatgctgtgaagaatcactggaactctaccatcaaaag

SEQ ID NO: 1355 - DNA, 85 bp, Artificial Sequence
  Synthetic Oligonucleotide
cggtacttctcagggctaatatatacgtactctggcctcttctgcgtggtggtcaacccctataaacacctgcccatctactcgg

SEQ ID NO: 1356 - DNA, 79 bp, Artificial Sequence
  Synthetic Oligonucleotide
gtgaggcagcgcgactctggcgactggccggccatgccttcccgggctgaggactatgaagtgttgtacaccattggca

SEQ ID NO: 1357 - DNA, 73 bp, Artificial Sequence
  Synthetic Oligonucleotide
cagaccaaggagatggacctcagcgtggtgcggctcatgtttacagcttttcttccggatagcactggcagct

SEQ ID NO: 1358 - DNA, 68 bp, Artificial Sequence
  Synthetic Oligonucleotide
ctgccgggatggcttctatgaggctgagctctgcccggaccgctgcatccacagtttccagaacctgg

SEQ ID NO: 1359 - DNA, 67 bp, Artificial Sequence
  Synthetic Oligonucleotide
cactgacacccgcaacaccacccatggttcggactctgtggtttcagccagcagagagattgcagcc

SEQ ID NO: 1360 - DNA, 66 bp, Artificial Sequence
  Synthetic Oligonucleotide
ctgcttctttcccgagcttggaacttcgttatccgcgatgcgtttcctggcagctacattcctgct

SEQ ID NO: 1361 - DNA, 73 bp, Artificial Sequence
  Synthetic Oligonucleotide
ggctgtggctgaggctgtagcatctctgctggaggtgagacactctgggaactgatttgacctcgaatgctcc

SEQ ID NO: 1362 - DNA, 69 bp, Artificial Sequence
  Synthetic Oligonucleotide
cggacctgaatgtaatgcaagaatgaacagaacaagcaaaatgaccagctgcttagtcacctggcaaag

SEQ ID NO: 1363 - DNA, 67 bp, Artificial Sequence
  Synthetic Oligonucleotide
caaaggaagagcaacggaagaaacgcgagcaagaacgaaaggagaagaaagcaaaggttttgggaat

SEQ ID NO: 1364 - DNA, 65 bp, Artificial Sequence
  Synthetic Oligonucleotide
tggagactctcagggtcgaaaacggcggcagaccagcatgacagatttctaccactccaaacgcc

SEQ ID NO: 1365 - DNA, 66 bp, Artificial Sequence
  Synthetic Oligonucleotide
cggtggaccacgaagagttaacccgggacttggagaagcactgcagagacatggaagaggcgagcc

SEQ ID NO: 1366 - DNA, 67 bp, Artificial Sequence
  Synthetic Oligonucleotide
tcactaccagctgacatccggctgcctgagggctacctggagaagctgaccctcaatagccccatct

SEQ ID NO: 1367 - DNA, 66 bp, Artificial Sequence
  Synthetic Oligonucleotide
ccagctctccttccagctacagatcaatgtccctgtccgagtgctggagctaagtgagagccaccc

SEQ ID NO: 1368 - DNA, 66 bp, Artificial Sequence
  Synthetic Oligonucleotide
gagaagcacgccatgaaacaggccgtcatggaaatgatgagtcagaagattcagcagctcacagcc

SEQ ID NO: 1369 - DNA, 74 bp, Artificial Sequence
  Synthetic Oligonucleotide
agagccagttgctgtagaactcaaatctctgctgggcaaggatgttctgttcttgaaggactgtgtaggcccag

SEQ ID NO: 1370 - DNA, 71 bp, Artificial Sequence
  Synthetic Oligonucleotide
gacattgtggtaggggaagggactcattttctcatcccgtgggtacagaaaccaattatctttgactgccg

SEQ ID NO: 1371 - DNA, 83 bp, Artificial Sequence
  Synthetic Oligonucleotide
ataccaatcaccgcacaaacccaggctatttgttaagtccagtcacagcgcaaagaaacatatgcggagaaaatgctagtgtg

SEQ ID NO: 1372 - DNA, 67 bp, Artificial Sequence
  Synthetic Oligonucleotide
ctgctcaaggacaccgtctacacggacttcgatgggacccgagtgtatagccctccagagtggatcc

SEQ ID NO: 1373 - DNA, 67 bp, Artificial Sequence
  Synthetic Oligonucleotide
tggggacattccctttgagagggaccaggagattctggaagctgagctccacttcccagcccatgtc

SEQ ID NO: 1374 - DNA, 76 bp, Artificial Sequence
  Synthetic Oligonucleotide
cccatggatgctcctctgaagagactttcctcattgactgccgaggccccatgaatcaatgtctggtagccaccgg

SEQ ID NO: 1375 - DNA, 66 bp, Artificial Sequence
  Synthetic Oligonucleotide
ccaagttctgggtggtggaccagacccacttctacctgggcagtgccaacatggactggcgttcac

SEQ ID NO: 1376 - DNA, 69 bp, Artificial Sequence
  Synthetic Oligonucleotide
aatgaatacagtattcccaagcacatcaaccccgtggccgcctccctcatccagaagatgcttcagaca

SEQ ID NO: 1377 - DNA, 77 bp, Artificial Sequence
  Synthetic Oligonucleotide
cttacggttttcgtggagaagccttggggtcaatttgttgtatagctgaggttttaattacaacaagaacggctgct

SEQ ID NO: 1378 - DNA, 75 bp, Artificial Sequence
  Synthetic Oligonucleotide
gatgtggactgccattcaaaccaggaagataccggatgtaaatttcgagttttgcctcagccaactaatctcgca

SEQ ID NO: 1379 - DNA, 66 bp, Artificial Sequence
  Synthetic Oligonucleotide
ccacataccgtccagcctatctactggagaacgaagaagaggagcggaactcccgcacatgacctc

SEQ ID NO: 1380 - DNA, 78 bp, Artificial Sequence
  Synthetic Oligonucleotide
gcaatcatggaacttgacgatactctaaaatactctttcttgcagtttgacccagcacctcgtagaggcgagccacat

SEQ ID NO: 1381 - DNA, 85 bp, Artificial Sequence
  Synthetic Oligonucleotide
gcatcaggctgtcattatggtgtccttacctgtgggagctgtaaggtcttctttaagagggcaatggaagggcagcacaactact

SEQ ID NO: 1382 - DNA, 67 bp, Artificial Sequence
  Synthetic Oligonucleotide
aggactgggacccatgaacattcctttggtatcagacccgaagcgcaccattgctcaggattatggg

SEQ ID NO: 1383 - DNA, 69 bp, Artificial Sequence
  Synthetic Oligonucleotide
ggtgtccttcgccagatcactgttaatgatttgcctgtgggacgctccgtggatgaggctctgcggctg

SEQ ID NO: 1384 - DNA, 70 bp, Artificial Sequence
  Synthetic Oligonucleotide
caagcaatgcgtcatcaatgtccccagcctctgcggaatggatcacactgagaagagggggcggatttac

SEQ ID NO: 1385 - DNA, 68 bp, Artificial Sequence
  Synthetic Oligonucleotide
ctgacacttgccgcagagaatccctttctcacccacctcatctgcaccttccagaccaaggaccacct

SEQ ID NO: 1386 - DNA, 66 bp, Artificial Sequence
  Synthetic Oligonucleotide
gggttctagacgcccctcccaagcgttcctggccttctgaactccatacagcctctacagccgtcc

SEQ ID NO: 1387 - DNA, 68 bp, Artificial Sequence
  Synthetic Oligonucleotide
ctccacctatgagcgtctgtctctgtgggcttgggatgttaacaggagccaaaaggagggaaagtgtg

SEQ ID NO: 1388 - DNA, 86 bp, Artificial Sequence
  Synthetic Oligonucleotide
gccctcccagtgtgcaaataagggctgctgtttcgacgacaccgttcgtggggtcccctggtgcttctatcctaataccatcgacg

SEQ ID NO: 1389 - DNA, 81 bp, Artificial Sequence
  Synthetic Oligonucleotide
tggctaagtgaagatgacaatcatgttgcagcaattcactgtaaagctggaaagggacgaactggtgtaatgatatgtgca

SEQ ID NO: 1390 - DNA, 81 bp, Artificial Sequence
  Synthetic Oligonucleotide
cgcttgcctaactcatactttcccgttgacacttgatccacgcagcgtggcactgggacgtaagtggcgcagtctgaatgg

SEQ ID NO: 1391 - DNA, 74 bp, Artificial Sequence
  Synthetic Oligonucleotide
ggctactctgatctatgttgataaggaaaatggagaaccaggcacccgtgtggttgctaaggatgggctgaagc

SEQ ID NO: 1392 - DNA, 65 bp, Artificial Sequence
  Synthetic Oligonucleotide
gggacactgcgggacaagagcggttccggagtctcaccactgcatttttcagagacgccatgggc

SEQ ID NO: 1393 - DNA, 67 bp, Artificial Sequence
  Synthetic Oligonucleotide
ctgaaggaccctacgctcggtggcctggcacctcactttgagaagagtgagcacactggctttgcat

SEQ ID NO: 1394 - DNA, 78 bp, Artificial Sequence
  Synthetic Oligonucleotide
gcgacagctcctctagttccaccatgtccgcgggcggagacttcgggaatccgctgaggaaattcaagctggtgttcc

SEQ ID NO: 1395 - DNA, 71 bp, Artificial Sequence
  Synthetic Oligonucleotide
gaggagtggtgacagtctgcaaaatcaatacacaggaacctgaggagaccctggactttgatttctgcagc

SEQ ID NO: 1396 - DNA, 67 bp, Artificial Sequence
  Synthetic Oligonucleotide
agctagcctcagtgacacacatgacaggttgcactgccgacgttgtgtcaacagccgtcagatccgg

SEQ ID NO: 1397 - DNA, 73 bp, Artificial Sequence
  Synthetic Oligonucleotide
cgtcgtatgcgagagtctgtttccaggatgcctgttagttctcagcacagatattctacacctcacgccttca

SEQ ID NO: 1398 - DNA, 84 bp, Artificial Sequence
  Synthetic Oligonucleotide
ggtgtcagatataaatgtgcaaatgccttcttgctgtcctgtcggtctcagtacgttcactttatagctgctggcaatatcgaa

SEQ ID NO: 1399 - DNA, 67 bp, Artificial Sequence
  Synthetic Oligonucleotide
tgtggatgctggattgatttcaccactggtgcagctgctaaatagcaaagaccaggaagtgctgctt

SEQ ID NO: 1400 - DNA, 69 bp, Artificial Sequence
  Synthetic Oligonucleotide
agtgggagacacctgacctttctcaagctgagattgagcagaagatcaaggagtacaatgcccagatca

SEQ ID NO: 1401 - DNA, 77 bp, Artificial Sequence
  Synthetic Oligonucleotide
cgaagcccttacaagtttcctagttcacccttacggattcctggagggaacatctatatttcacccctgaagagtcc

SEQ ID NO: 1402 - DNA, 66 bp, Artificial Sequence
  Synthetic Oligonucleotide
cccagtgtacgaggaacaagacagaccgagatctccaaccggacctagcaactccttcctcgctaa

SEQ ID NO: 1403 - DNA, 66 bp, Artificial Sequence
  Synthetic Oligonucleotide
gggctgggtgagaatgtgatggagaggaagaagccggccctggtatccattccggaggatgttgtg

SEQ ID NO: 1404 - DNA, 68 bp, Artificial Sequence
  Synthetic Oligonucleotide
cctacaagtcctggggcattggagccccaagcagtgttaatcctggctactgtgtgagcctgacctca

SEQ ID NO: 1405 - DNA, 67 bp, Artificial Sequence
  Synthetic Oligonucleotide
gcgaggagctctacttgctctgcgacaaggtgcagaaagaggacatatcagtggtgttcagcagggc

SEQ ID NO: 1406 - DNA, 67 bp, Artificial Sequence
  Synthetic Oligonucleotide
aagcatgaacaggacttgaccatctttccaacccctggggaagacatttgcaactgacttggggagg

SEQ ID NO: 1407 - DNA, 68 bp, Artificial Sequence
  Synthetic Oligonucleotide
cccgttcggtctgaggaaggccgggacatggcgaaccggatcagtgcctttggctaccttgagtgctc

SEQ ID NO: 1408 - DNA, 74 bp, Artificial Sequence
  Synthetic Oligonucleotide
ccagacgagcgattagaagcggcagcttgtgaggtgaatgatttgggggaagaggaggaggaggaagaggagga

SEQ ID NO: 1409 - DNA, 73 bp, Artificial Sequence
  Synthetic Oligonucleotide
tgtgcacataggaatgagcttcagatgcagttggccagcaaagagagtgatattgagcaattgcgtgctaaac

SEQ ID NO: 1410 - DNA, 74 bp, Artificial Sequence
  Synthetic Oligonucleotide
gatctggcactgtggttcctgcatgaagacagtggctggcggtgcctggacgtacaataccacttccgctgtca

SEQ ID NO: 1411 - DNA, 75 bp, Artificial Sequence
  Synthetic Oligonucleotide
ccattctatcatcaacgggtacaaacgagtcctggccttgtctgtggagacggattacaccttcccacttgctga

SEQ ID NO: 1412 - DNA, 67 bp, Artificial Sequence
  Synthetic Oligonucleotide
ctgtcttcctgttggccctgacaatcatagccagcacctgggctctgacgcccactcactacctcac

SEQ ID NO: 1413 - DNA, 81 bp, Artificial Sequence
  Synthetic Oligonucleotide
gctcattatgaaaaacatcccaaactttaaaatgcgaaattattggttggtgtgaagaaagccagacaacttctgtttctt

SEQ ID NO: 1414 - DNA, 66 bp, Artificial Sequence
  Synthetic Oligonucleotide
gctctgttgtgtcctgttgccggctctggccttcctgtgactgactgtgaagtggcttctccgtac

SEQ ID NO: 1415 - DNA, 71 bp, Artificial Sequence
  Synthetic Oligonucleotide
cgaggagatgcctgtggacaggatcctggaggcagagcttgctgtggaacagaagagtgaccagggcgttg

SEQ ID NO: 1416 - DNA, 77 bp, Artificial Sequence
  Synthetic Oligonucleotide
acaccaaaatgccatctcaaatggaacacgccatggaaaccatgatgtttacatttcacaaattcgctggggataaa

SEQ ID NO: 1417 - DNA, 67 bp, Artificial Sequence
  Synthetic Oligonucleotide
cttctgagcccgtctcccggacaggttgaggaagctgctccagaagcgcctcggaaggggagctctc

SEQ ID NO: 1418 - DNA, 86 bp, Artificial Sequence
  Synthetic Oligonucleotide
cgcgagcccctcattatacactgggcagcctccccacagcgcatcgaggaatgcgtgctctcaggcaaggatgtcaacggcgagtg

SEQ ID NO: 1419 - DNA, 70 bp, Artificial Sequence
  Synthetic Oligonucleotide
gagagagccagtctgatccagaaggccaagctggcagagcaggccgaacgctatgaggacatggcagcct

SEQ ID NO: 1420 - DNA, 77 bp, Artificial Sequence
  Synthetic Oligonucleotide
cagtggagaccagttgggtagtggtgactgggtacgctacaagctctgcatgtgtgctgatgggacgctcttcaagg

SEQ ID NO: 1421 - DNA, 73 bp, Artificial Sequence
  Synthetic Oligonucleotide
tccgcaagacacctcctggagggcctcctgcagaaggacaggacaaagcggctcggggccaaggatgacttca

SEQ ID NO: 1422 - DNA, 71 bp, Artificial Sequence
  Synthetic Oligonucleotide
tgcattcgttggtttctcttatgcacctccttcagaagacttatttttgtgagcagtttgccattcagaaa

SEQ ID NO: 1423 - DNA, 71 bp, Artificial Sequence
  Synthetic Oligonucleotide
ccaacaccttcttggcttctgggacctgtgttcttgctgagcaccctctccggtttgggttgggataacag

SEQ ID NO: 1424 - DNA, 72 bp, Artificial Sequence
  Synthetic Oligonucleotide
agctggggtgtctgtttcatgtggaatacctgacttcaggtcaagggatggtatttatgctcgccttgctgt

SEQ ID NO: 1425 - DNA, 67 bp, Artificial Sequence
  Synthetic Oligonucleotide
gtggggagcccatcatctcgccaagccatcacaggctctgcatacacatgcactcagtgtggactgg

SEQ ID NO: 1426 - DNA, 64 bp, Artificial Sequence
  Synthetic Oligonucleotide
tctgccagtgctgaattctttgctgacattgccctggctcctatggaagctgctaaggttcgaa

SEQ ID NO: 1427 - DNA, 67 bp, Artificial Sequence
  Synthetic Oligonucleotide
cccaactcaggtccttggtaaatcctgcaagccaatcccagtcatgctccttggggtgaccctcttg

SEQ ID NO: 1428 - DNA, 70 bp, Artificial Sequence
  Synthetic Oligonucleotide
agatgcatacttggaagcacagtcatatcacactgggaggcaatgcaatgtggttacctggtcctaggtt

SEQ ID NO: 1429 - DNA, 70 bp, Artificial Sequence
  Synthetic Oligonucleotide
gcgcagaggccagttaaagtagatcacctcctcgaacccactccggttccccgcaacccacagctcagct

SEQ ID NO: 1430 - DNA, 76 bp, Artificial Sequence
  Synthetic Oligonucleotide
ggctggccaaacataagcagctgcactgcgatgcccagtctagaaaatctttcagctgtaaatactgtgacaagga

SEQ ID NO: 1431 - DNA, 66 bp, Artificial Sequence
  Synthetic Oligonucleotide
agtgacaaatgttggaggagcagtggtgacgggtgtgacagcagtagcccagaagacagtggaggg

SEQ ID NO: 1432 - DNA, 80 bp, Artificial Sequence
  Synthetic Oligonucleotide
acccaccatggatgtcttcaagaagggcttctccatcgccaaggagggcgtggtgggtgcggtggaaaagaccaagcagg

SEQ ID NO: 1433 - DNA, 70 bp, Artificial Sequence
  Synthetic Oligonucleotide
tgaagagaggcatgttggagacttgggcaatgtgactgctgacaaagatggtgtggccgatgtgtctatt

SEQ ID NO: 1434 - DNA, 74 bp, Artificial Sequence
  Synthetic Oligonucleotide
tgaggagtggtattttggcaagatcaccagacgggagtcagagcggttactgctcaatgcagagaacccgagag

SEQ ID NO: 1435 - DNA, 74 bp, Artificial Sequence
  Synthetic Oligonucleotide
atacagcaccaatggaaagatcaccttcgacgactacatcgcctgctgcgtcaaactgagggctcttacagaca

SEQ ID NO: 1436 - DNA, 81 bp, Artificial Sequence
  Synthetic Oligonucleotide
gggctcagctttcagaagtgctgagttggcagttttcttctgtcaccaaaagaggtctcaatgtggaccagctgaacatgt

SEQ ID NO: 1437 - DNA, 70 bp, Artificial Sequence
  Synthetic Oligonucleotide
tcacatgccactttggtgtttcataatctcctgggagagattgaccagcagtatagccgcttcctgcaag

SEQ ID NO: 1438 - DNA, 76 bp, Artificial Sequence
  Synthetic Oligonucleotide
caagagggactcggactgcagcagcctctgcacctctgagagcatggactatggtaccaatctctccactgacctg

SEQ ID NO: 1439 - DNA, 66 bp, Artificial Sequence
  Synthetic Oligonucleotide
ggactcggagacgctgtgcaggagggccgtcaagatcctcaagaagaagaagttgcgaaggatccc

SEQ ID NO: 1440 - DNA, 69 bp, Artificial Sequence
  Synthetic Oligonucleotide
catcttccaggaggaccactctctgtggcaccctggactacctgccccctgaaatgattgaaggtcgga

SEQ ID NO: 1441 - DNA, 71 bp, Artificial Sequence
  Synthetic Oligonucleotide
aatacccaacgcacaaatgaccgcacgttctctgccccgtttcttgccccagtgtggtttgcattgtctcc

SEQ ID NO: 1442 - DNA, 90 bp, Artificial Sequence
  Synthetic Oligonucleotide
cctggaggctgcaacatacctcaatcctgtcccaggccggatcctcctgaagcccttttcgcagcactgctatcctccaaagccattgta

SEQ ID NO: 1443 - DNA, 80 bp, Artificial Sequence
  Synthetic Oligonucleotide
tgttttgattcccgggcttaccaggtgagaagtgagggaggaagaaggcagtgtcccttttgctagagctgacagctttg

SEQ ID NO: 1444 - DNA, 65 bp, Artificial Sequence
  Synthetic Oligonucleotide
cacccttggactggaaaactcacacccggtctggacacagaaagagaaccaacagctcatcaagg

SEQ ID NO: 1445 - DNA, 66 bp, Artificial Sequence
  Synthetic Oligonucleotide
gatcctcgcgtgagacagatcaagatcaagaccggcgtggtgaagcggttggtcaaagaaaaagtg

SEQ ID NO: 1446 - DNA, 68 bp, Artificial Sequence
  Synthetic Oligonucleotide
ctgttttcctggaggactgcagtgactgcgtgctggcagtggcctgccaacagctccgcatacacagt

SEQ ID NO: 1447 - DNA, 66 bp, Artificial Sequence
  Synthetic Oligonucleotide
cagccaggtgtacgagacattgctcacctacagtgacgtcgtgggcgcggatgtgctggacgaggt

SEQ ID NO: 1448 - DNA, 66 bp, Artificial Sequence
  Synthetic Oligonucleotide
tcccgagagaggaaagcatgatgggagccacgaagggactgtgtattttaaatgcaggcacccgac

SEQ ID NO: 1449 - DNA, 74 bp, Artificial Sequence
  Synthetic Oligonucleotide
cctggttgaagcctgttaatgctttcaacgtgtggaaaacccagcgggcctttagcaaatatgagaagtctgca

SEQ ID NO: 1450 - DNA, 68 bp, Artificial Sequence
  Synthetic Oligonucleotide
ccagtgtgtgtaacagggtcacaagaattcgacagccagatgctccaagagggtggcccaaggctata

SEQ ID NO: 1451 - DNA, 68 bp, Artificial Sequence
  Synthetic Oligonucleotide
gccaactgctttcatttgtgagggatctgaaccaatacagagcagacataaaggaaatgggcctgagt

SEQ ID NO: 1452 - DNA, 85 bp, Artificial Sequence
  Synthetic Oligonucleotide
catccgcaaagtgactgaagagaacaaagagttggccaatgagctgaggcggcctcccctatgctatcacaacggagttcagtac

SEQ ID NO: 1453 - DNA, 84 bp, Artificial Sequence
  Synthetic Oligonucleotide
gccgggaagaccgtaattgtggctgcactggatgggaccttccagaggaagccatttggggccatcctgaacctggtgccgctg

SEQ ID NO: 1454 - DNA, 72 bp, Artificial Sequence
  Synthetic Oligonucleotide
aatccaagggggagagtgatgacttccatatggactttgactcagctgtggctcctcgggcaaaatctgtac

SEQ ID NO: 1455 - DNA, 66 bp, Artificial Sequence
  Synthetic Oligonucleotide
gtgatgccttccctgtgggcgaggtgaagatgctggagaagcagacgaacccacccgactacctga

SEQ ID NO: 1456 - DNA, 82 bp, Artificial Sequence
  Synthetic Oligonucleotide
ctatatgcagccagagatgtgacagccaccgtggacagcctgccactcatcacagcctccattctcagtaagaaactcgtgg

SEQ ID NO: 1457 - DNA, 74 bp, Artificial Sequence
  Synthetic Oligonucleotide
tgctgttgctgagtctgttgccagtccccagaagaccatgtctgtgttgagctgtatctgtgaagccaggcaag

SEQ ID NO: 1458 - DNA, 81 bp, Artificial Sequence
  Synthetic Oligonucleotide
ggtgtcgatattgtcatgaaccatcacctgcaggaaacaagtttcacaaaagaagcctacaagaagtacatcaaagattac

SEQ ID NO: 1459 - DNA, 66 bp, Artificial Sequence
  Synthetic Oligonucleotide
gacgctggtctggtgaagatgtccaggaaaccacgagcctccagcccattgtccaacaaccaccca

SEQ ID NO: 1460 - DNA, 73 bp, Artificial Sequence
  Synthetic Oligonucleotide
cttcacagtgctcctgcagtctctctgtgtggctgtaacttacgtgtactttaccaacgagctgaagcagatg

SEQ ID NO: 1461 - DNA, 65 bp, Artificial Sequence
  Synthetic Oligonucleotide
gcctcggtgtgcctttcaacatcgccagctacgccctgctcacgtacatgattgcgcacatcacg

SEQ ID NO: 1462 - DNA, 66 bp, Artificial Sequence
  Synthetic Oligonucleotide
ctggtcagccttcagggaccctgagcaccgcctggtctctttcctgtggccagcccagaactgaag

SEQ ID NO: 1463 - DNA, 76 bp, Artificial Sequence
  Synthetic Oligonucleotide
tgcttgtcagttgtcaacaccttatggccaacctgcctgtttccagcagcaacagcatcaaatacttgccactcca

SEQ ID NO: 1464 - DNA, 68 bp, Artificial Sequence
  Synthetic Oligonucleotide
tgtcaccccgactcaacgtgagacgcaccgcccggactcaccatgcgtgaatgcatctcagtccacgt

SEQ ID NO: 1465 - DNA, 62 bp, Artificial Sequence
  Synthetic Oligonucleotide
agctcaacatgcgtgagtgtatctctatccacgtggggcaggcaggagtccagatcggcaat

SEQ ID NO: 1466 - DNA, 71 bp, Artificial Sequence
  Synthetic Oligonucleotide
ctcttacatcgaccgcctaagagtcgcgctgtaagaagcaacaacctctcctcttcgtctccgccatcagc

SEQ ID NO: 1467 - DNA, 71 bp, Artificial Sequence
  Synthetic Oligonucleotide
gaggagggtgagttctccaaggcccatgaggatatgactgccctggagaaggattacaaggaggtgggcat

SEQ ID NO: 1468 - DNA, 71 bp, Artificial Sequence
  Synthetic Oligonucleotide
gtcccttcgcctccttcaccgccgcagaccccttcaagttctagtcatgcgtgagtgcatctccatccacg

SEQ ID NO: 1469 - DNA, 68 bp, Artificial Sequence
  Synthetic Oligonucleotide
cgccctacctataccaacctcaaccgcctcatcagtcagattgtgtcctcaatcactgcttctctccg

SEQ ID NO: 1470 - DNA, 73 bp, Artificial Sequence
  Synthetic Oligonucleotide
cgaggacgaggcttaaaaacttctcagatcaatcgtgcatccttagtgaacttctgttgtcctcaagcatggt

SEQ ID NO: 1471 - DNA, 70 bp, Artificial Sequence
  Synthetic Oligonucleotide
cgccctcctgcagtatttatggcctcgtcctcccccacctaggccacgtgtgagctgctcctgtctctgt

SEQ ID NO: 1472 - DNA, 84 bp, Artificial Sequence
  Synthetic Oligonucleotide
acactgactggcatcctgctttccagtgcctgccagcctccagaagagccaggtgcctgactagtacatggggagctacagagc

SEQ ID NO: 1473 - DNA, 70 bp, Artificial Sequence
  Synthetic Oligonucleotide
gtggcctagagccttcagtcactggggaaagcagggaagcagtgtgaactctttattcactcccagcctg

SEQ ID NO: 1474 - DNA, 73 bp, Artificial Sequence
  Synthetic Oligonucleotide
acaggccccatgcatcctccctgcctcactcccctcagcccctgccgaccttagcttatctgggagagaaaca

SEQ ID NO: 1475 - DNA, 80 bp, Artificial Sequence
  Synthetic Oligonucleotide
ccctatggccctgaatggtgcactggtttaattgtgttggtgtcggcccctcacaaatgcagccaagtcatgtaattagt

SEQ ID NO: 1476 - DNA, 75 bp, Artificial Sequence
  Synthetic Oligonucleotide
agtggaatccttccctttccaactctacctccctcactcagctcctttcccctgatcagagaaagggatcaaggg

SEQ ID NO: 1477 - DNA, 75 bp, Artificial Sequence
  Synthetic Oligonucleotide
ggaaggaaagaagcatggtctactttaggtgtgcgctgggtctctggtgctcttcactgttgcctgtcacttttt

SEQ ID NO: 1478 - DNA, 67 bp, Artificial Sequence
  Synthetic Oligonucleotide
gatgccgagggaaatcatcaccctacagttgggccagtgcggcaatcagattgggttcgagttctgg

SEQ ID NO: 1479 - DNA, 64 bp, Artificial Sequence
  Synthetic Oligonucleotide
gcgctgcggaagatcatccccacgctgccctcggacaagctgagcaagattcagaccctcaagc

SEQ ID NO: 1480 - DNA, 67 bp, Artificial Sequence
  Synthetic Oligonucleotide
cagtgtggaggggatggaggagcctgacatccagtgggtgaaggatggggctgtggtccagaacttg

SEQ ID NO: 1481 - DNA, 69 bp, Artificial Sequence
  Synthetic Oligonucleotide
agttgtcgtgtgttctggattcattccggcaccaccatgtcgaaggtttcctttaagatcacgctgacg

SEQ ID NO: 1482 - DNA, 70 bp, Artificial Sequence
  Synthetic Oligonucleotide
gtggatgtgccctgaaggacaagccaggcgtctacacgagagtctcacacttcttaccctggatccgcag

SEQ ID NO: 1483 - DNA, 89 bp, Artificial Sequence
  Synthetic Oligonucleotide
tggcttcaggagctgaataccctcccaggcacacacaggtgggacacaaataagggttttggaaccactattttctcatcacgacagca

SEQ ID NO: 1484 - DNA, 71 bp, Artificial Sequence
  Synthetic Oligonucleotide
ctgctgtcttgggtgcattggagccttgccttgctgctctacctccaccatgccaagtggtcccaggctgc

SEQ ID NO: 1485 - DNA, 71 bp, Artificial Sequence
  Synthetic Oligonucleotide
tgacgatggcctggagtgtgtgcccactgggcagcaccaagtccggatgcagatcctcatgatccggtacc

SEQ ID NO: 1486 - DNA, 83 bp, Artificial Sequence
  Synthetic Oligonucleotide
cctcagcaagacgttatttgaaattacagtgcctctctctcaaggccccaaaccagtaacaatcagttttgccaatcacactt

SEQ ID NO: 1487 - DNA, 67 bp, Artificial Sequence
  Synthetic Oligonucleotide
cggttggtgacttgtctgcctcctgctttgggaagactgaggcatccgtgaggcagggacaagtctt

SEQ ID NO: 1488 - DNA, 72 bp, Artificial Sequence
  Synthetic Oligonucleotide
tgcccttaaaggaaccaatgagtccctggaacgccagatgcgtgaaatggaagagaactttgccgttgaagc

SEQ ID NO: 1489 - DNA, 84 bp, Artificial Sequence
  Synthetic Oligonucleotide
ggttgtgctctacgagcttatgactggctcactgccttacagccacattggctgccgtgaccagattatctttatggtgggccg

SEQ ID NO: 1490 - DNA, 68 bp, Artificial Sequence
  Synthetic Oligonucleotide
ctctccagtgtgggcaccagccggccagaacagatgcgagcagtccatgactctgggagctacaccgc

SEQ ID NO: 1491 - DNA, 75 bp, Artificial Sequence
  Synthetic Oligonucleotide
gtatcaggaccacatgcagtacatcggagaaggcgcgaagacaggcatcaaagaatgccagtatcaattccgaca

SEQ ID NO: 1492 - DNA, 77 bp, Artificial Sequence
  Synthetic Oligonucleotide
gcagttggaagacacaggaaagtatccccaaattgcagatttatcaacggcttttatcttgaaaatagtgccacgca

SEQ ID NO: 1493 - DNA, 75 bp, Artificial Sequence
  Synthetic Oligonucleotide
caggtcaggcagaggaagtcatgtgcattgcatgagctaaacctatctgaatgaattgatttggggcttgttagg

SEQ ID NO: 1494 - DNA, 66 bp, Artificial Sequence
  Synthetic Oligonucleotide
tggtcagatgctgctgaagtatatccttaggccgctggcatcttgcccatcccttcatgctgtgat

SEQ ID NO: 1495 - DNA, 66 bp, Artificial Sequence
  Synthetic Oligonucleotide
gagggagcagacagtgggtaccacgatctccgtaaccatttgcatgtgacttagcaagggctctga

SEQ ID NO: 1496 - DNA, 66 bp, Artificial Sequence
  Synthetic Oligonucleotide
tagaggccatcaaaattggcctcaccaaggccctgactcagatggaggaagcccagaggaaacgga